(19) United States
(12) Patent Application Publication
(10) Pub. No.: US 2023/0416361 A1
(43) Pub. Date: Dec. 28, 2023

(54) ENGINEERED CD200R ANTIBODIES AND USES THEREOF

(71) Applicants: MiroBio Limited, Oxford (GB); Oxford University Innovation Limited, Oxford (GB); Gilead Sciences, Inc., Foster City, CA (US)

(72) Inventors: Simon John DAVIS, Oxford (GB); Richard John CORNALL, Oxford (GB); Christopher Douglas PALUCH, Oxford (GB); Lynne Anne MURRAY, Oxford (GB); Nathan Jacob ROBERTSON, Oxford (GB); Eleanor Marysia SCOTT, Oxford (GB); Daniela M. TOMAZELA, Belmont, CA (US)

(21) Appl. No.: 18/296,296

(22) Filed: Apr. 5, 2023

Related U.S. Application Data
(60) Provisional application No. 63/328,015, filed on Apr. 6, 2022.
(60) Provisional application No. 63/348,532, filed on Jun. 3, 2022.

(51) Int. Cl. C07K 16/28; A61K 45/06; A61K 39/395; A61K 47/68; A61P 37/06

(52) U.S. Cl. CPC C07K 16/2803; A61K 45/06; A61K 39/3955; A61K 47/6849; A61P 37/06; C07K 2317/64; C07K 2317/569

(57) ABSTRACT

In some aspects, provided herein are antibodies or antigen-binding fragments that bind to CD200R, a glycoprotein receptor present on cell surfaces. Antibodies or antigen-binding fragments provided herein, in some cases, agonize CD200R signaling pathway that inhibits inflammation and immune response. In other aspects, provided herein are compositions, methods of use, methods of making, polynucleotides, vectors, host cells, and kits relating to antibodies or antigen-binding fragments that bind to CD200R.

### CROSS-REFERENCE TO RELATED APPLICATIONS

This application claims the benefit under 35 U.S.C. § 119(e) of U.S. Provisional Application No. 63/328,015, filed on Apr. 6, 2022, and U.S. Provisional Application No. 63/348,532, filed on Jun. 3, 2022, which are hereby incorporated herein by reference in their entireties for all purposes.

### SEQUENCE LISTING

The instant application contains a Sequence Listing which has been submitted electronically in .XML file format and is hereby incorporated by reference in its entirety. Said .XML copy, created on Apr. 1, 2023, is named 210196-209004_US.XML and is 112,067 bytes in size.

### SUMMARY

Disclosed herein, in some aspects, is an antibody or an antigen-binding fragment thereof that specifically binds CD200R, comprising a heavy chain that comprises at least one heavy chain complementarity determining region (CDR) as set forth in any of SEQ ID NO: 3, 4, 41, 5, 11, 12, 13, 19, 20, 21, 27, 28, 29, 35, 36, 37, 69, 70, or 91, with from 0 to 3 amino acid modifications.

Disclosed herein, in some aspects, is an antibody or an antigen-binding fragment thereof that specifically binds CD200R, comprising a heavy chain and a light chain, wherein the heavy chain comprises a heavy chain variable region that comprises heavy chain complementarity determining region 1 (CDRH1), CDRH2, and CDRH3, and wherein CDRH1, CDRH2, and CDRH3 comprise the sequence as set forth in (a) SEQ ID NOs: 3, 41, and 70, respectively, each with 0 to 3 amino acid modifications, such as 0, 1, 2, or 3 modifications; (b) SEQ ID NOs: 3, 41, and 69, respectively, each with 0 to 3 amino acid modifications, such as 0, 1, 2, or 3 modifications; (c) SEQ ID NOs: 3, 4, and 5, respectively, each with 0 to 3 amino acid modifications, such as 0, 1, 2, or 3 modifications; (d) SEQ ID NOs: 3, 41, and 5, respectively, each with 0 to 3 amino acid modifications, such as 0, 1, 2, or 3 modifications; (e) SEQ ID NOs: 11, 12, and 13, respectively, each with 0 to 3 amino acid modifications, such as 0, 1, 2, or 3 modifications; (f) SEQ ID NOs: 19, 20, and 21, respectively, each with 0 to 3 amino acid modifications, such as 0, 1, 2, or 3 modifications; (g) SEQ ID NOs: 27, 28, and 29, respectively, each with 0 to 3 amino acid modifications, such as 0, 1, 2, or 3 modifications; or (h) SEQ ID NOs: 35, 36, and 37, respectively, each with 0 to 3 amino acid modifications, such as 0, 1, 2, or 3 modifications.

Disclosed herein, in some aspects, is an antibody or an antigen-binding fragment thereof that specifically binds CD200R, comprising a light chain that comprises at least one light chain complementarity determining region (CDR) as set forth in any of SEQ ID NO: 6, 7, 8, 14, 15, 16, 22, 23, 24, 30, 31, 32, 38, 39, 40, 67, 68, or 87 to 91, with from 0 to 3 amino acid modifications.

Disclosed herein, in some aspects, is an antibody or an antigen-binding fragment thereof, comprising a heavy chain and a light chain, wherein the light chain comprises a light chain variable region that comprises light chain complementarity determining region 1 (CDRL1), CDRL2, and CDRL3, and wherein CDRL1, CDRL2, and CDRL3 comprise the sequence as set forth in (a) SEQ ID NOs: 6, 7, and 8, respectively, each with 0 to 3 amino acid modifications, such as 0, 1, 2, or 3 modifications; (b) SEQ ID NOs: 14, 15, and 16, respectively, each with 0 to 3 amino acid modifications, such as 0, 1, 2, or 3 modifications; (c) SEQ ID NOs: 22, 23, and 24, respectively, each with 0 to 3 amino acid modifications, such as 0, 1, 2, or 3 modifications; (d) SEQ ID NOs: 30, 31, and 32, respectively, each with 0 to 3 amino acid modifications, such as 0, 1, 2, or 3 modifications; (e) SEQ ID NOs: 38, 39, and 40, respectively, each with 0 to 3 amino acid modifications, such as 0, 1, 2, or 3 modifications; (f) SEQ ID NOs: 6, 67, and 8, respectively, each with 0 to 3 amino acid modifications, such as 0, 1, 2, or 3 modifications; or (g) SEQ ID NOs: 6, 68, and 8, respectively, each with 0 to 3 amino acid modifications, such as 0, 1, 2, or 3 modifications.

Disclosed herein, in some aspects, is an antibody or an antigen-binding fragment thereof that specifically binds CD200R, comprising: (a) a heavy chain that comprises at least one heavy chain CDR as set forth in any of SEQ ID NO: 3, 4, 41, 5, 69, or 70, with from 0 to 3 amino acid modifications, and a light chain that comprises at least one light chain CDR as set forth in any of SEQ ID NO: 6, 7, or 8, with from 0 to 3 amino acid modifications; (b) a heavy chain that comprises at least one heavy chain CDR as set forth in any of SEQ ID NO: 3, 4, 41, 5, 69, or 70, with from 0 to 3 amino acid modifications, and a light chain that comprises at least one light chain CDR as set forth in any of SEQ ID NO: 6, 67, or 8, with from 0 to 3 amino acid modifications; (c) a heavy chain that comprises at least one heavy chain CDR as set forth in any of SEQ ID NO: 3, 4, 41, 5, 69, or 70, with from 0 to 3 amino acid modifications, and a light chain that comprises at least one light chain CDR as set forth in any of SEQ ID NO: 6, 68, or 8, with from 0 to 3 amino acid modifications; (d) a heavy chain that comprises at least one heavy chain CDR as set forth in any of SEQ ID NO: 3, 41, or 70, with from 0 to 3 amino acid modifications, and a light chain that comprises at least one light chain CDR as set forth in any of SEQ ID NO: 6, 67, or 8, with from 0 to 3 amino acid modifications; (e) a heavy chain that comprises at least one heavy chain CDR as set forth in any of SEQ ID NO: 3, 41, or 69, with from 0 to 3 amino acid modifications, and a light chain that comprises at least one light chain CDR as set forth in any of SEQ ID NO: 6, 67, or 8, with from 0 to 3 amino acid modifications; (f) a heavy chain that comprises at least one heavy chain CDR as set forth in any of SEQ ID NO: 11, 12, or 13, with from 0 to 3 amino acid modifications, and a light chain that comprises at least one light chain CDR as set forth in any of SEQ ID NO: 14, 15, or 16, with from 0 to 3 amino acid modifications; (g) a heavy chain that comprises at least one heavy chain CDR as set forth in any of SEQ ID NO: 19, 20, or 21, with from 0 to 3 amino acid modifications, and a light chain that comprises at least one light chain CDR as set forth in any of SEQ ID NO: 22, 23, or 24, with from 0 to 3 amino acid modifications; (h) a heavy chain that comprises at least one heavy chain CDR as set forth in any of SEQ ID NO: 27, 28, or 29, with from 0 to 3 amino acid modifications, and a light chain that comprises at least one light chain CDR as set forth in any of SEQ ID NO: 30, 31, or 32, with from 0 to 3 amino acid modifications; or (i) a heavy chain that comprises at least one heavy chain CDR as set forth in any of SEQ ID NO: 35, 36, or 37, with from 0 to 3 amino acid modifications, and a light chain that comprises at least one light chain CDR as set forth in any of SEQ ID NO: 38, 39, or 40, with from 0 to 3 amino acid modifications.

Disclosed herein, in some aspects, is an antibody or an antigen-binding fragment thereof, comprising a heavy chain and a light chain, wherein: (1) the heavy chain comprises a heavy chain variable region that comprises heavy chain complementarity determining region 1 (CDRH1), CDRH2, and CDRH3, wherein CDRH1, CDRH2, and CDRH3 comprise the sequence as set forth in SEQ ID NOs: 3, 41, and 70, respectively, each with 0 to 3 amino acid modifications, such as 0, 1, 2, or 3 modifications; and the light chain comprises a light chain variable region that comprises light chain complementarity determining region 1 (CDRL1), CDRL2, and CDRL3, wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications; (2) the heavy chain comprises a heavy chain variable region that comprises heavy chain complementarity determining region 1 (CDRH1), CDRH2, and CDRH3, wherein CDRH1, CDRH2, and CDRH3 comprise the sequence as set forth in SEQ ID NOs: 3, 41, and 5, respectively, each with 0 to 3 amino acid modifications, such as 0, 1, 2, or 3 modifications; and the light chain comprises a light chain variable region that comprises light chain complementarity determining region 1 (CDRL1), CDRL2, and CDRL3, wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications; (3) the heavy chain comprises a heavy chain variable region that comprises heavy chain complementarity determining region 1 (CDRH1), CDRH2, and CDRH3, wherein CDRH1, CDRH2, and CDRH3 comprise the sequence as set forth in SEQ ID NOs: 3, 4, and 5, respectively, each with 0 to 3 amino acid modifications, such as 0, 1, 2, or 3 modifications; and the light chain comprises a light chain variable region that comprises light chain complementarity determining region 1 (CDRL1), CDRL2, and CDRL3, wherein CDRL1, CDRL2, and CDRL3 comprise the sequence as set forth in SEQ ID NOs: 6, 7, and 8, respectively, each with 0 to 3 amino acid modifications, such as 0, 1, 2, or 3 modifications; (4) the heavy chain comprises a heavy chain variable region that comprises heavy chain CDRH1, CDRH2, and CDRH3, and wherein CDRH1, CDRH2, and CDRH3 comprise the sequence as set forth in SEQ ID NOs: 3, 41, and 5, respectively, each with 0 to 3 amino acid modifications, such as 0, 1, 2, or 3 modifications; and the light chain comprises a light chain variable region that comprises CDRL1, CDRL2, and CDRL3, and wherein CDRL1, CDRL2, and CDRL3 comprise the sequence as set forth in SEQ ID NOs: 6, 7, and 8, respectively, each with 0 to 3 amino acid modifications, such as 0, 1, 2, or 3 modifications; (5) the heavy chain comprises a heavy chain variable region that comprises CDRH1, CDRH2, and CDRH3, and wherein CDRH1, CDRH2, and CDRH3 comprise the sequence as set forth in SEQ ID NOs: 11, 12, and 13, respectively, each with 0 to 3 amino acid modifications, such as 0, 1, 2, or 3 modifications; and the light chain comprises a light chain variable region that comprises CDRL1, CDRL2, and CDRL3, and wherein CDRL1, CDRL2, and CDRL3 comprise the sequence as set forth in SEQ ID NOs: 14, 15, and 16, respectively, each with 0 to 3 amino acid modifications, such as 0, 1, 2, or 3 modifications; (6) the heavy chain comprises a heavy chain variable region that comprises CDRH1, CDRH2, and CDRH3, and wherein CDRH1, CDRH2, and CDRH3 comprise the sequence as set forth in SEQ ID NOs: 19, 20, and 21, respectively, each with 0 to 3 amino acid modifications, such as 0, 1, 2, or 3 modifications; and the light chain comprises a light chain variable region that comprises CDRL1, CDRL2, and CDRL3, and wherein CDRL1, CDRL2, and CDRL3 comprise the sequence as set forth in SEQ ID NOs: 22, 23, and 24, respectively, each with 0 to 3 amino acid modifications, such as 0, 1, 2, or 3 modifications; (7) the heavy chain comprises a heavy chain variable region that comprises CDRH1, CDRH2, and CDRH3, and wherein CDRH1, CDRH2, and CDRH3 comprise the sequence as set forth in SEQ ID NOs: 27, 28, and 29, respectively, each with 0 to 3 amino acid modifications, such as 0, 1, 2, or 3 modifications; and the light chain comprises a light chain variable region that comprises CDRL1, CDRL2, and CDRL3, and wherein CDRL1, CDRL2, and CDRL3 comprise the sequence as set forth in SEQ ID NOs: 30, 31, and 32, respectively, each with 0 to 3 amino acid modifications, such as 0, 1, 2, or 3 modifications; (8) the heavy chain comprises a heavy chain variable region that comprises CDRH1, CDRH2, and CDRH3, and wherein CDRH1, CDRH2, and CDRH3 comprise the sequence as set forth in SEQ ID NOs: 35, 36, and 37, respectively, each with 0 to 3 amino acid modifications, such as 0, 1, 2, or 3 modifications; and the light chain comprises a light chain variable region that comprises CDRL1, CDRL2, and CDRL3, and wherein CDRL1, CDRL2, and CDRL3 comprise the sequence as set forth in SEQ ID NOs: 38, 39, and 40, respectively, each with 0 to 3 amino acid modifications, such as 0, 1, 2, or 3 modifications; (9) the heavy chain comprises a heavy chain variable region that comprises heavy chain complementarity determining region 1 (CDRH1), CDRH2, and CDRH3, wherein CDRH1, CDRH2, and CDRH3 comprise the sequence as set forth in SEQ ID NOs: 3, 4, and 5, respectively, each with 0 to 3 amino acid modifications, such as 0, 1, 2, or 3 modifications; and the light chain comprises a light chain variable region that comprises light chain complementarity determining region 1 (CDRL1), CDRL2, and CDRL3, wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications; or (10) the heavy chain comprises a heavy chain variable region that comprises heavy chain complementarity determining region 1 (CDRH1), CDRH2, and CDRH3, wherein CDRH1, CDRH2, and CDRH3 comprise the sequence as set forth in SEQ ID NOs: 3, 4, and 5, respectively, each with 0 to 3 amino acid modifications, such as 0, 1, 2, or 3 modifications; and the light chain comprises a light chain variable region that comprises light chain complementarity determining region 1 (CDRL1), CDRL2, and CDRL3, wherein CDRL1, CDRL2, and CDRL3 comprise the sequence as set forth in SEQ ID NOs: 6, 68, and 8, respectively, each with 0 to 3 amino acid modifications, such as 0, 1, 2, or 3 modifications.

Disclosed herein, in some aspects, is an antibody or an antigen-binding fragment thereof, comprising a heavy chain and a light chain, wherein: the heavy chain comprises a heavy chain variable region that comprises heavy chain complementarity determining region 1 (CDRH1), CDRH2, and CDRH3, wherein CDRH1, CDRH2, and CDRH3 comprise the sequence as set forth in SEQ ID NOs: 3, 41, and 69, respectively, each with 0 to 3 amino acid modifications, such as 0, 1, 2, or 3 modifications; and the light chain comprises a light chain variable region that comprises light chain complementarity determining region 1 (CDRL1), CDRL2, and CDRL3, wherein CDRL1, CDRL2, and CDRL3 comprise the sequence as set forth in SEQ ID NOs: 6, 90, and 8, respectively, each with 0 to 3 amino acid modifications, such as 0, 1, 2, or 3 modifications. In some embodiments, X at position 1 of SEQ ID NO: 69 is M. In some embodiments, X at position 1 of SEQ ID NO: 69 is G. In some embodiments, X at position 7 of SEQ ID NO: 90 is D.

Disclosed herein is an antibody or antigen-binding fragment thereof, wherein the antibody or antigen-binding fragment thereof comprises a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 70, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some embodiments, the HCVR comprises an amino acid sequence having at least 90%, 95%, 96%, 97%, 98%, 99%, or 100% sequence identity to the amino acid sequence as set forth in SEQ ID NO: 72, and the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 72, with from 0 to 10 amino acid modifications, such as 1, 2, 3, 4, 5, 6, 7, 8, 9 or 10 amino acid modifications, and the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications.

Disclosed herein is an antibody or antigen-binding fragment thereof, wherein the antibody or antigen-binding fragment thereof comprises a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 5, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some embodiments, the HCVR comprises an amino acid sequence having at least 90%, 95%, 96%, 97%, 98%, 99%, or 100% sequence identity to the amino acid sequence as set forth in SEQ ID NO: 71, and the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 71, with from 0 to 10 amino acid modifications, such as 1, 2, 3, 4, 5, 6, 7, 8, 9 or 10 amino acid modifications, and the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications.

Disclosed herein, in some aspects, is an antibody or an antigen-binding fragment thereof, comprising a heavy chain and a light chain, wherein: (a) the heavy chain comprises a heavy chain variable region that comprises (i) a heavy chain complementarity determining region 1 (CDRH1) comprising the sequence as set forth in SEQ ID NO: 92, (ii) a CDRH2 comprising the sequence as set forth in SEQ ID NO: 41, and (iii) a CDRH3 comprising the sequence as set forth in SEQ ID NO: 69; and (b) the light chain comprises a light chain variable region that comprises (i) a light chain complementarity determining region 1 (CDRL1) comprising the sequence as set forth in SEQ ID NO: 6, (ii) a CDRL2 comprising a sequence selected from SEQ ID NOs: 87 to 90, and (iii) a CDRL3 comprising the sequence as set forth in SEQ ID NO: 91. In some embodiments, X at position 1 of SEQ ID NO: 69 is M In some embodiments, X at position 1 of SEQ ID NO: 69 is G. In some embodiments, X at position 8 of SEQ ID NO: 91 is W. In some embodiments, X at position 8 of SEQ ID NO: 91 is F. In some embodiments, X at position 3 of SEQ ID NO: 92 is W. In some embodiments, X at position 3 of SEQ ID NO: 92 is F. In some embodiments, X at position 1 of SEQ ID NO: 87, 88, or 89 is G. In some embodiments, X at position 1 of SEQ ID NO: 87, 88, or 89 is L. In some embodiments, X at position 2 of SEQ ID NO: 87 or 90 is A. In some embodiments, X at position 2 of SEQ ID NO: 87 or 90 is G. In some embodiments, X at position 3 of SEQ ID NO: 87 or 88 is S. In some embodiments, X at position 3 of SEQ ID NO: 87 or 88 is V. In some embodiments, X at position 7 of SEQ ID NO: 87, 88, 89, or 90 is D. In some embodiments, X at position 7 of SEQ ID NO: 87, 88, 89, or 90 is S. In some embodiments, X at position 7 of SEQ ID NO: 87, 88, 89, or 90 is T. In some embodiments, the HCVR comprises an amino acid sequence having at least 90%, 95%, 96%, 97%, 98%, 99%, or 100% sequence identity to the amino acid sequence as set forth in SEQ ID NO: 72, and the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 72, with from 0 to 10 amino acid modifications, and the LCVR comprises an amino acid sequence as set forth in SEQ ID NO: 65, with from 0 to 10 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 71, and the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 71, with from 0 to 10 amino acid modifications, and the LCVR comprises an amino acid sequence as set forth in SEQ ID NO: 65, with from 0 to 10 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 45, and the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 45, with from 0 to 10 amino acid modifications, and the LCVR comprises an amino acid sequence as set forth in SEQ ID NO: 65, with from 0 to 10 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 93, and the LCVR comprises an amino acid sequence having at least 90%, 95%, 96%, 97%, 98%, 99%, or 100% sequence identity to the amino acid sequence as set forth in SEQ ID NO: 94. In some embodiments, the HCVR comprises an amino acid sequence as set forth in SEQ ID NO: 93, with from 0 to 10 amino acid modifications, such as from 0, 1, 2, 3, 4, 5, 6, 7, 8, 9, or 10 modifications, and the LCVR comprises an amino acid sequence as set forth in SEQ ID NO: 94, with from 0 to 10 amino acid modifications, such as from 0, 1, 2, 3, 4, 5, 6, 7, 8, 9, or 10 modifications. In some embodiments, X at position 1 of SEQ ID NO: 93 is D. In some embodiments, X at position 1 of SEQ ID NO: 93 is E. In some embodiments, X at position 33 of SEQ ID NO: 93 is W. In some embodiments, X at position 33 of SEQ ID NO: 93 is F. In some embodiments, X at position 99 of SEQ ID NO: 93 is M. In some embodiments, X at position 99 of SEQ ID NO: 93 is G. In some embodiments, X at position 50 of SEQ ID NO: 94 is G. In some embodiments, X at position 50 of SEQ ID NO: 94 is L. In some embodiments, X at position 51 of SEQ ID NO: 94 is A. In some embodiments, X at position 51 of SEQ ID NO: 94 is G. In some embodiments, X at position 52 of SEQ ID NO: 94 is S. In some embodiments, X at position 52 of SEQ ID NO: 94 is V. In some embodiments, X at position 56 of SEQ ID NO: 94 is D. In some embodiments, X at position 56 of SEQ ID NO: 94 is S. In some embodiments, X at position 56 of SEQ ID NO: 94 is T. In some embodiments, X at position 96 of SEQ ID NO: 94 is W. In some embodiments, X at position 96 of SEQ ID NO: 94 is F. In some embodiments, the LCVR is linked to a light chain constant region comprising an amino acid sequence having at least 90%, 95%, 96%, 97%, 98%, 99%, or 100% sequence identity to the amino acid sequence as set forth in SEQ ID NO: 62. In some embodiments, the light chain variable region is linked to a light chain constant region comprising an amino acid sequence as set forth in SEQ ID NO: 62, with from 0 to 10 amino acid modifications. In some embodiments, the light chain variable region is linked to a light chain constant region comprising an amino acid sequence having at least 90%, 95%, 96%, 97%, 98%, 99%, or 100% sequence identity to the amino acid sequence as set forth in SEQ ID NO: 64. In some embodiments, the light chain variable region is linked to a light chain constant region comprising an amino acid sequence as set forth in SEQ ID NO: 64, with from 0 to 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 61. In some embodiments, the HCVR is linked to a heavy chain constant region comprising an amino acid sequence as set forth in SEQ ID NO: 61, with from 0 to 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 63. In some embodiments, the HCVR is linked to a heavy chain constant region comprising an amino acid sequence as set forth in SEQ ID NO: 63, with from 0 to 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 75. In some embodiments, the HCVR is linked to a heavy chain constant region comprising an amino acid sequence as set forth in SEQ ID NO: 75, with from 0 to 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 76. In some embodiments, the HCVR is linked to a heavy chain constant region comprising an amino acid sequence as set forth in SEQ ID NO: 76, with from 0 to 10 amino acid modifications. In some embodiments, the HCVR is linked to a heavy chain constant region, wherein the heavy chain constant region comprises an Fc region that comprises one or more of the following amino acids: alanine (A) at position 234, alanine (A) at position 235, aspartic acid (D) at position 236, aspartic acid (D) at position 237, aspartic acid (D) at position 238, alanine (A) at position 265, glutamic acid (E) at position 267, glycine (G) at position 271, arginine (R) at position 330, alanine (A) at position 332, or alanine (A) at position 297 (numbering according to EU Index). In some embodiments, the Fc region comprises an aspartic acid (D) at position 238 (EU Index). In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 77. In some embodiments, the HCVR is linked to a heavy chain constant region comprising an amino acid sequence as set forth in SEQ ID NO: 77, with from 0 to 10 amino acid modifications. In some embodiments, X at position 121 of SEQ ID NO: 77 is D. In some embodiments, X at position 121 of SEQ ID NO: 77 is P. In some embodiments, X at position 329 of SEQ ID NO: 77 is G. In some embodiments, X at position 329 of SEQ ID NO: 77 is absent. In some embodiments, X at position 330 of SEQ ID NO: 77 is K. In some embodiments, X at position 330 of SEQ ID NO: 77 is absent. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 78. In some embodiments, the HCVR is linked to a heavy chain constant region comprising an amino acid sequence as set forth in SEQ ID NO: 78, with from 0 to 10 amino acid modifications. In some embodiments, X at position 108 of SEQ ID NO: 78 is S. In some embodiments, X at position 108 of SEQ ID NO: 78 is P. In some embodiments, X at position 326 of SEQ ID NO: 78 is G. In some embodiments, X at position 326 of SEQ ID NO: 78 is absent. In some embodiments, X at position 327 of SEQ ID NO: 78 is K. In some embodiments, X at position 327 of SEQ ID NO: 78 is absent.

Disclosed herein, in some aspects, is an antibody or an antigen-binding fragment thereof, comprising a heavy chain and a light chain, wherein: the heavy chain comprises a heavy chain variable region that comprises heavy chain complementarity determining region 1 (CDRH1), CDRH2, and CDRH3, wherein CDRH1, CDRH2, and CDRH3 comprise the sequence as set forth in SEQ ID NOs: 3, 41, and 69, respectively, each with 0 to 3 amino acid modifications; and the light chain comprises a light chain variable region that comprises light chain complementarity determining region 1 (CDRL1), CDRL2, and CDRL3, wherein CDRL1, CDRL2, and CDRL3 comprise the sequence as set forth in SEQ ID NOs: 6, 90, and 8, respectively, each with 0 to 3 amino acid modifications. In some embodiments, X at position 1 of SEQ ID NO: 69 is M. In some embodiments, X at position 1 of SEQ ID NO: 69 is G. In some embodiments, X at position 7 of SEQ ID NO: 90 is D. In some embodiments, X at position 7 of SEQ ID NO: 90 is S. In some embodiments, X at position 7 of SEQ ID NO: 90 is T. In some embodiments, the HCVR comprises an amino acid sequence having at least 90%, 95%, 96%, 97%, 98%, 99%, or 100% sequence identity to the amino acid sequence as set forth in SEQ ID NO: 72, and the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 72, with from 0 to 10 amino acid modifications, and the LCVR comprises an amino acid sequence as set forth in SEQ ID NO: 65, with from 0 to 10 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 71, and the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 71, with from 0 to 10 amino acid modifications, and the LCVR comprises an amino acid sequence as set forth in SEQ ID NO: 65, with from 0 to 10 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 45, and the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 45, with from 0 to 10 amino acid modifications, and the LCVR comprises an amino acid sequence as set forth in SEQ ID NO: 65, with from 0 to 10 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 93, and the LCVR comprises an amino acid sequence having at least 90%, 95%, 96%, 97%, 98%, 99%, or 100% sequence identity to the amino acid sequence as set forth in SEQ ID NO: 94. In some embodiments, the HCVR comprises an amino acid sequence as set forth in SEQ ID NO: 93, with from 0 to 10 amino acid modifications, such as from 0, 1, 2, 3, 4, 5, 6, 7, 8, 9, or 10 modifications, and the LCVR comprises an amino acid sequence as set forth in SEQ ID NO: 94, with from 0 to 10 amino acid modifications, such as from 0, 1, 2, 3, 4, 5, 6, 7, 8, 9, or 10 modifications. In some embodiments, X at position 1 of SEQ ID NO: 93 is D. In some embodiments, X at position 1 of SEQ ID NO: 93 is E. In some embodiments, X at position 33 of SEQ ID NO: 93 is W. In some embodiments, X at position 33 of SEQ ID NO: 93 is F. In some embodiments, X at position 99 of SEQ ID NO: 93 is M. In some embodiments, X at position 99 of SEQ ID NO: 93 is G. In some embodiments, X at position 50 of SEQ ID NO: 94 is G. In some embodiments, X at position 50 of SEQ ID NO: 94 is L. In some embodiments, X at position 51 of SEQ ID NO: 94 is A. In some embodiments, X at position 51 of SEQ ID NO: 94 is G. In some embodiments, X at position 52 of SEQ ID NO: 94 is S. In some embodiments, X at position 52 of SEQ ID NO: 94 is V. In some embodiments, X at position 56 of SEQ ID NO: 94 is D. In some embodiments, X at position 56 of SEQ ID NO: 94 is S. In some embodiments, X at position 56 of SEQ ID NO: 94 is T. In some embodiments, X at position 96 of SEQ ID NO: 94 is W. In some embodiments, X at position 96 of SEQ ID NO: 94 is F. In some embodiments, the LCVR is linked to a light chain constant region comprising an amino acid sequence having at least 90%, 95%, 96%, 97%, 98%, 99%, or 100% sequence identity to the amino acid sequence as set forth in SEQ ID NO: 62. In some embodiments, the light chain variable region is linked to a light chain constant region comprising an amino acid sequence as set forth in SEQ ID NO: 62, with from 0 to 10 amino acid modifications. In some embodiments, the light chain variable region is linked to a light chain constant region comprising an amino acid sequence having at least 90%, 95%, 96%, 97%, 98%, 99%, or 100% sequence identity to the amino acid sequence as set forth in SEQ ID NO: 64. In some embodiments, the light chain variable region is linked to a light chain constant region comprising an amino acid sequence as set forth in SEQ ID NO: 64, with from 0 to 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 61. In some embodiments, the HCVR is linked to a heavy chain constant region comprising an amino acid sequence as set forth in SEQ ID NO: 61, with from 0 to 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 63. In some embodiments, the HCVR is linked to a heavy chain constant region comprising an amino acid sequence as set forth in SEQ ID NO: 63, with from 0 to 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 75. In some embodiments, the HCVR is linked to a heavy chain constant region comprising an amino acid sequence as set forth in SEQ ID NO: 75, with from 0 to 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 76. In some embodiments, the HCVR is linked to a heavy chain constant region comprising an amino acid sequence as set forth in SEQ ID NO: 76, with from 0 to 10 amino acid modifications. In some embodiments, the HCVR is linked to a heavy chain constant region, wherein the heavy chain constant region comprises an Fc region that comprises one or more of the following amino acids: alanine (A) at position 234, alanine (A) at position 235, aspartic acid (D) at position 236, aspartic acid (D) at position 237, aspartic acid (D) at position 238, alanine (A) at position 265, glutamic acid (E) at position 267, glycine (G) at position 271, arginine (R) at position 330, alanine (A) at position 332, or alanine (A) at position 297 (numbering according to EU Index). In some embodiments, the Fc region comprises an aspartic acid (D) at position 238 (EU Index). In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 77. In some embodiments, the HCVR is linked to a heavy chain constant region comprising an amino acid sequence as set forth in SEQ ID NO: 77, with from 0 to 10 amino acid modifications. In some embodiments, X at position 121 of SEQ ID NO: 77 is D. In some embodiments, X at position 121 of SEQ ID NO: 77 is P. In some embodiments, X at position 329 of SEQ ID NO: 77 is G. In some embodiments, X at position 329 of SEQ ID NO: 77 is absent. In some embodiments, X at position 330 of SEQ ID NO: 77 is K. In some embodiments, X at position 330 of SEQ ID NO: 77 is absent. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 78. In some embodiments, the HCVR is linked to a heavy chain constant region comprising an amino acid sequence as set forth in SEQ ID NO: 78, with from 0 to 10 amino acid modifications. In some embodiments, X at position 108 of SEQ ID NO: 78 is S. In some embodiments, X at position 108 of SEQ ID NO: 78 is P. In some embodiments, X at position 326 of SEQ ID NO: 78 is G. In some embodiments, X at position 326 of SEQ ID NO: 78 is absent. In some embodiments, X at position 327 of SEQ ID NO: 78 is K. In some embodiments, X at position 327 of SEQ ID NO: 78 is absent.

In some cases, the antibody or antigen-binding fragment thereof comprises a heavy chain variable region that comprises an amino acid sequence having at least 90%, 95%, 96%, 97%, 98%, 99%, or 100% sequence identity to the amino acid sequence as set forth in SEQ ID NO: 1, 9, 17, 25, 33, 42, 43, 44, 45, 46, 47, 71, 72, or 93.

In some cases, the antibody or antigen-binding fragment thereof comprises a heavy chain variable region that comprises an amino acid sequence as set forth in SEQ ID NO: 1, 9, 17, 25, 33, 42, 43, 44, 45, 46, 47, 71, 72, or 93, with from 0 to 3 amino acid modifications.

In some cases, the antibody or antigen-binding fragment thereof comprises a light chain variable region that comprises an amino acid sequence having at least 90%, 95%, 96%, 97%, 98%, 99%, or 100% sequence identity to the amino acid sequence as set forth in SEQ ID NO: 2, 10, 18, 26, 34, 48, 49, 50, 51, 52, 65, 66, or 94.

In some cases, the antibody or antigen-binding fragment thereof comprises a light chain variable region that comprises an amino acid sequence as set forth in SEQ ID NO: 2, 10, 18, 26, 34, 48, 49, 50, 51, 52, 65, 66, or 94, with from 0 to 3 amino acid modifications.

In some cases, the antibody or antigen-binding fragment thereof comprises a heavy chain variable region and a light chain variable region, wherein: (a) the heavy chain variable region comprises an amino acid sequence having at least 90%, 95%, 96%, 97%, 98%, 99%, or 100% sequence identity to the amino acid sequence as set forth in SEQ ID NO: 72, and the light chain variable region comprises an amino acid sequence having at least 90%, 95%, 96%, 97%, 98%, 99%, or 100% sequence identity to the amino acid sequence as set forth in SEQ ID NO: 65; (b) the heavy chain variable region comprises an amino acid sequence having at least 90%, 95%, 96%, 97%, 98%, 99%, or 100% sequence identity to the amino acid sequence as set forth in SEQ ID NO: 71, and the light chain variable region comprises an amino acid sequence having at least 90%, 95%, 96%, 97%, 98%, 99%, or 100% sequence identity to the amino acid sequence as set forth in SEQ ID NO: 65; (c) the heavy chain variable region comprises an amino acid sequence having at least 90%, 95%, 96%, 97%, 98%, 99%, or 100% sequence identity to the amino acid sequence as set forth in SEQ ID NO: 1, and the light chain variable region comprises an amino acid sequence having at least 90%, 95%, 96%, 97%, 98%, 99%, or 100% sequence identity to the amino acid sequence as set forth in SEQ ID NO: 2; (d) the heavy chain variable region comprises an amino acid sequence having at least 90%, 95%, 96%, 97%, 98%, 99%, or 100% sequence identity to the amino acid sequence as set forth in SEQ ID NO: 9, and the light chain variable region comprises an amino acid sequence having at least 90%, 95%, 96%, 97%, 98%, 99%, or 100% sequence identity to the amino acid sequence as set forth in SEQ ID NO: 10; (e) the heavy chain variable region comprises an amino acid sequence having at least 90%, 95%, 96%, 97%, 98%, 99%, or 100% sequence identity to the amino acid sequence as set forth in SEQ ID NO: 17, and the light chain variable region comprises an amino acid sequence having at least 90%, 95%, 96%, 97%, 98%, 99%, or 100% sequence identity to the amino acid sequence as set forth in SEQ ID NO: 18; (f) the heavy chain variable region comprises an amino acid sequence having at least 90%, 95%, 96%, 97%, 98%, 99%, or 100% sequence identity to the amino acid sequence as set forth in SEQ ID NO: 25, and the light chain variable region comprises an amino acid sequence having at least 90%, 95%, 96%, 97%, 98%, 99%, or 100% sequence identity to the amino acid sequence as set forth in SEQ ID NO: 26; (g) the heavy chain variable region comprises an amino acid sequence having at least 90%, 95%, 96%, 97%, 98%, 99%, or 100% sequence identity to the amino acid sequence as set forth in SEQ ID NO: 33, and the light chain variable region comprises an amino acid sequence having at least 90%, 95%, 96%, 97%, 98%, 99%, or 100% sequence identity to the amino acid sequence as set forth in SEQ ID NO: 34; (h) the heavy chain variable region comprises an amino acid sequence having at least 90%, 95%, 96%, 97%, 98%, 99%, or 100% sequence identity to the amino acid sequence as set forth in any one of SEQ ID NOs: 1, 42, 43, 44, 45, 46, 47, 71, 72, or 93 and the light chain variable region comprises an amino acid sequence having at least 90%, 95%, 96%, 97%, 98%, 99%, or 100% sequence identity to the amino acid sequence as set forth in SEQ ID NO: 2, 48, 49, 50, 51, 52, 65, 66, or 94; or (i) the heavy chain variable region comprises an amino acid sequence having at least 90%, 95%, 96%, 97%, 98%, 99%, or 100% sequence identity to the amino acid sequence as set forth in any one of SEQ ID NOs: 42, 43, 44, 45, 46, 47, 71, 72, or 93 and the light chain variable region comprises an amino acid sequence having at least 90%, 95%, 96%, 97%, 98%, 99%, or 100% sequence identity to the amino acid sequence as set forth in SEQ ID NOS: 48, 49, 50, 51, 52, 65, 66, or 94.

In some cases, the antibody or antigen-binding fragment thereof comprises a heavy chain variable region and a light chain variable region, wherein: (a) the heavy chain variable region comprises an amino acid sequence as set forth in SEQ ID NO: 72, with from 0 to 10 amino acid modifications, such as 1, 2, 3, 4, 5, 6, 7, 8, 9 or 10 amino acid modifications, and the light chain variable region comprises an amino acid sequence as set forth in SEQ ID NO: 65, with from 0 to 10 amino acid modifications, such as 1, 2, 3, 4, 5, 6, 7, 8, 9 or 10 amino acid modifications; (b) the heavy chain variable region comprises an amino acid sequence as set forth in SEQ ID NO: 71, with from 0 to 10 amino acid modifications, such as 1, 2, 3, 4, 5, 6, 7, 8, 9 or 10 amino acid modifications, and the light chain variable region comprises an amino acid sequence as set forth in SEQ ID NO: 65, with from 0 to 10 amino acid modifications, such as 1, 2, 3, 4, 5, 6, 7, 8, 9 or 10 amino acid modifications; (c) the heavy chain variable region comprises an amino acid sequence as set forth in SEQ ID NO: 1, with from 0 to 10 amino acid modifications, such as 1, 2, 3, 4, 5, 6, 7, 8, 9 or 10 amino acid modifications, and the light chain variable region comprises an amino acid sequence as set forth in SEQ ID NO: 2, with from 0 to 10 amino acid modifications, such as 1, 2, 3, 4, 5, 6, 7, 8, 9 or 10 amino acid modifications; (d) the heavy chain variable region comprises an amino acid sequence as set forth in SEQ ID NO: 9, with from 0 to 10 amino acid modifications, such as 1, 2, 3, 4, 5, 6, 7, 8, 9 or 10 amino acid modifications, and the light chain variable region comprises an amino acid sequence as set forth in SEQ ID NO: 10, with from 0 to 10 amino acid modifications, such as 1, 2, 3, 4, 5, 6, 7, 8, 9 or 10 amino acid modifications; (e) the heavy chain variable region comprises an amino acid sequence as set forth in SEQ ID NO: 17, with from 0 to 10 amino acid modifications, such as 1, 2, 3, 4, 5, 6, 7, 8, 9 or 10 amino acid modifications, and the light chain variable region comprises an amino acid sequence as set forth in SEQ ID NO: 18, with from 0 to 10 amino acid modifications, such as 1, 2, 3, 4, 5, 6, 7, 8, 9 or 10 amino acid modifications; (f) the heavy chain variable region comprises an amino acid sequence as set forth in SEQ ID NO: 25, with from 0 to 10 amino acid modifications, such as 1, 2, 3, 4, 5, 6, 7, 8, 9 or 10 amino acid modifications, and the light chain variable region comprises an amino acid sequence as set forth in SEQ ID NO: 26, with from 0 to 10 amino acid modifications, such as 1, 2, 3, 4, 5, 6, 7, 8, 9 or 10 amino acid modifications; (g) the heavy chain variable region comprises an amino acid sequence as set forth in SEQ ID NO: 33, with from 0 to 10 amino acid modifications, such as 1, 2, 3, 4, 5, 6, 7, 8, 9 or 10 amino acid modifications, and the light chain variable region comprises an amino acid sequence as set forth in SEQ ID NO: 34, with from 0 to 10 amino acid modifications, such as 1, 2, 3, 4, 5, 6, 7, 8, 9 or 10 amino acid modifications; (h) the heavy chain variable region comprises an amino acid sequence as set forth in any one of SEQ ID NOs: 1, 42, 43, 44, 45, 46, 47, 71, 72, or 93 with from 0 to 10 amino acid modifications, such as 1, 2, 3, 4, 5, 6, 7, 8, 9 or 10 amino acid modifications, and the light chain variable region comprises an amino acid sequence as set forth in SEQ ID NOs: 2, 48, 49, 50, 51, 52, 65, 66, or 94, with from 0 to 10 amino acid modifications, such as 1, 2, 3, 4, 5, 6, 7, 8, 9 or 10 amino acid modifications; or (i) the heavy chain variable region comprises an amino acid sequence as set forth in any one of SEQ ID NOs: 42, 43, 44, 45, 46, 47, 71, 72, or 93 with from 0 to 10 amino acid modifications, such as 1, 2, 3, 4, 5, 6, 7, 8, 9 or 10 amino acid modifications, and the light chain variable region comprises an amino acid sequence as set forth in SEQ ID NOs: 48, 49, 50, 51, 52, 65, 66, or 94 with from 0 to 10 amino acid modifications, such as 1, 2, 3, 4, 5, 6, 7, 8, 9 or 10 amino acid modifications.

Disclosed herein, in some aspects, is an antibody or an antigen-binding fragment thereof of that specifically binds CD200R, wherein the antibody or antigen-binding fragment thereof comprises a heavy chain that comprises a heavy chain variable region, and wherein the heavy chain variable region comprises an amino acid sequence having at least 90%, 95%, 96%, 97%, 98%, 99%, or 100% sequence identity to the amino acid sequence as set forth in SEQ ID NO: 1, 9, 17, 25, 33, 42, 43, 44, 45, 46, 47, 71, 72, or 93.

Disclosed herein, in some aspects, is an antibody or an antigen-binding fragment thereof of that specifically binds CD200R, wherein the antibody or antigen-binding fragment thereof comprises a heavy chain variable region, and wherein the heavy chain variable region comprises an amino acid sequence as set forth in SEQ ID NO: 1, 9, 17, 25, 33, 42, 43, 44, 45, 46, 47, 71, 72, or 93with from 0 to 10 amino acid modifications, such as 1, 2, 3, 4, 5, 6, 7, 8, 9 or 10 amino acid modifications.

Disclosed herein, in some aspects, is an antibody or an antigen-binding fragment thereof of that specifically binds CD200R, wherein the antibody or antigen-binding fragment thereof comprises a light chain that comprises a light chain variable region, and wherein the light chain variable region comprises an amino acid sequence having at least 90%, 95%, 96%, 97%, 98%, 99%, or 100% sequence identity to the amino acid sequence as set forth in SEQ ID NO: 2, 10, 18, 26, 34, 48, 49, 50, 51, 52, 65, 66, or 94.

Disclosed herein, in some aspects, is an antibody or an antigen-binding fragment thereof of that specifically binds CD200R, wherein the antibody or antigen-binding fragment thereof comprises a light chain that comprises a light chain variable region, and wherein the light chain variable region comprises an amino acid sequence as set forth in SEQ ID NO: 2, 10, 18, 26, 34, 48, 49, 50, 51, 52, 65, 66, or 94, with from 0 to 10 amino acid modifications, such as 1, 2, 3, 4, 5, 6, 7, 8, 9 or 10 amino acid modifications.

Disclosed herein, in some aspects, is an antibody or an antigen-binding fragment thereof of that specifically binds CD200R, comprising a heavy chain and a light chain, wherein the heavy chain comprises a heavy chain variable region, and the light chain comprises a light chain variable region, wherein: (a) the heavy chain variable region comprises an amino acid sequence having at least 90%, 95%, 96%, 97%, 98%, 99%, or 100% sequence identity to the amino acid sequence as set forth in SEQ ID NO: 72, and the light chain variable region comprises an amino acid sequence having at least 90%, 95%, 96%, 97%, 98%, 99%, or 100% sequence identity to the amino acid sequence as set forth in SEQ ID NO: 65; (b) the heavy chain variable region comprises an amino acid sequence having at least 90%, 95%, 96%, 97%, 98%, 99%, or 100% sequence identity to the amino acid sequence as set forth in SEQ ID NO: 71, and the light chain variable region comprises an amino acid sequence having at least 90%, 95%, 96%, 97%, 98%, 99%, or 100% sequence identity to the amino acid sequence as set forth in SEQ ID NO: 65; (c) the heavy chain variable region comprises an amino acid sequence having at least 90%, 95%, 96%, 97%, 98%, 99%, or 100% sequence identity to the amino acid sequence as set forth in SEQ ID NO: 1, and the light chain variable region comprises an amino acid sequence having at least 90%, 95%, 96%, 97%, 98%, 99%, or 100% sequence identity to the amino acid sequence as set forth in SEQ ID NO: 2; (d) the heavy chain variable region comprises an amino acid sequence having at least 90%, 95%, 96%, 97%, 98%, 99%, or 100% sequence identity to the amino acid sequence as set forth in SEQ ID NO: 9, and the light chain variable region comprises an amino acid sequence having at least 90%, 95%, 96%, 97%, 98%, 99%, or 100% sequence identity to the amino acid sequence as set forth in SEQ ID NO: 10; (e) the heavy chain variable region comprises an amino acid sequence having at least 90%, 95%, 96%, 97%, 98%, 99%, or 100% sequence identity to the amino acid sequence as set forth in SEQ ID NO: 17, and the light chain variable region comprises an amino acid sequence having at least 90%, 95%, 96%, 97%, 98%, 99%, or 100% sequence identity to the amino acid sequence as set forth in SEQ ID NO: 18; (f) the heavy chain variable region comprises an amino acid sequence having at least 90%, 95%, 96%, 97%, 98%, 99%, or 100% sequence identity to the amino acid sequence as set forth in SEQ ID NO: 25, and the light chain variable region comprises an amino acid sequence having at least 90%, 95%, 96%, 97%, 98%, 99%, or 100% sequence identity to the amino acid sequence as set forth in SEQ ID NO: 26; (g) the heavy chain variable region comprises an amino acid sequence having at least 90%, 95%, 96%, 97%, 98%, 99%, or 100% sequence identity to the amino acid sequence as set forth in SEQ ID NO: 33, and the light chain variable region comprises an amino acid sequence having at least 90%, 95%, 96%, 97%, 98%, 99%, or 100% sequence identity to the amino acid sequence as set forth in SEQ ID NO: 34; (h) the heavy chain variable region comprises an amino acid sequence having at least 90%, 95%, 96%, 97%, 98%, 99%, or 100% sequence identity to the amino acid sequence as set forth in any one of SEQ ID NOs: 1, 42, 43, 44, 45, 46, 47, 71, 72, or 93 and the light chain variable region comprises an amino acid sequence having at least 90%, 95%, 96%, 97%, 98%, 99%, or 100% sequence identity to the amino acid sequence as set forth in SEQ ID NO: 2, 48, 49, 50, 51, 52, 65, 66, or 94; or (i) the heavy chain variable region comprises an amino acid sequence having at least 90%, 95%, 96%, 97%, 98%, 99%, or 100% sequence identity to the amino acid sequence as set forth in any one of SEQ ID NOs: 42, 43, 44, 45, 46, 47, 71, 72, or 93 and the light chain variable region comprises an amino acid sequence having at least 90%, 95%, 96%, 97%, 98%, 99%, or 100% sequence identity to the amino acid sequence as set forth in SEQ ID NOS: 48, 49, 50, 51, 52, 65, 66, or 94.

Disclosed herein, in some aspects, is an antibody or an antigen-binding fragment thereof of that specifically binds CD200R, comprising a heavy chain and a light chain, wherein the heavy chain comprises a heavy chain variable region and the light chain comprises a light chain variable region, wherein: (a) the heavy chain variable region comprises an amino acid sequence as set forth in SEQ ID NO: 72, with from 0 to 10 amino acid modifications, such as 1, 2, 3, 4, 5, 6, 7, 8, 9 or 10 amino acid modifications, and the light chain variable region comprises an amino acid sequence as set forth in SEQ ID NO: 65, with from 0 to 10 amino acid modifications, such as 1, 2, 3, 4, 5, 6, 7, 8, 9 or 10 amino acid modifications; (b) the heavy chain variable region comprises an amino acid sequence as set forth in SEQ ID NO: 71, with from 0 to 10 amino acid modifications, such as 1, 2, 3, 4, 5, 6, 7, 8, 9 or 10 amino acid modifications, and the light chain variable region comprises an amino acid sequence as set forth in SEQ ID NO: 65, with from 0 to 10 amino acid modifications, such as 1, 2, 3, 4, 5, 6, 7, 8, 9 or 10 amino acid modifications; (c) the heavy chain variable region comprises an amino acid sequence as set forth in SEQ ID NO: 1, with from 0 to 10 amino acid modifications, such as 1, 2, 3, 4, 5, 6, 7, 8, 9 or 10 amino acid modifications, and the light chain variable region comprises an amino acid sequence as set forth in SEQ ID NO: 2, with from 0 to 10 amino acid modifications, such as 1, 2, 3, 4, 5, 6, 7, 8, 9 or 10 amino acid modifications; (d) the heavy chain variable region comprises an amino acid sequence as set forth in SEQ ID NO: 9, with from 0 to 10 amino acid modifications, such as 1, 2, 3, 4, 5, 6, 7, 8, 9 or 10 amino acid modifications, and the light chain variable region comprises an amino acid sequence as set forth in SEQ ID NO: 10, with from 0 to 10 amino acid modifications, such as 1, 2, 3, 4, 5, 6, 7, 8, 9 or 10 amino acid modifications; (e) the heavy chain variable region comprises an amino acid sequence as set forth in SEQ ID NO: 17, with from 0 to 10 amino acid modifications, such as 1, 2, 3, 4, 5, 6, 7, 8, 9 or 10 amino acid modifications, and the light chain variable region comprises an amino acid sequence as set forth in SEQ ID NO: 18, with from 0 to 10 amino acid modifications, such as 1, 2, 3, 4, 5, 6, 7, 8, 9 or 10 amino acid modifications; (f) the heavy chain variable region comprises an amino acid sequence as set forth in SEQ ID NO: 25, with from 0 to 10 amino acid modifications, such as 1, 2, 3, 4, 5, 6, 7, 8, 9 or 10 amino acid modifications, and the light chain variable region comprises an amino acid sequence as set forth in SEQ ID NO: 26, with from 0 to 10 amino acid modifications, such as 1, 2, 3, 4, 5, 6, 7, 8, 9 or 10 amino acid modifications; (g) the heavy chain variable region comprises an amino acid sequence as set forth in SEQ ID NO: 33, with from 0 to 10 amino acid modifications, such as 1, 2, 3, 4, 5, 6, 7, 8, 9 or 10 amino acid modifications, and the light chain variable region comprises an amino acid sequence as set forth in SEQ ID NO: 34, with from 0 to 10 amino acid modifications, such as 1, 2, 3, 4, 5, 6, 7, 8, 9 or 10 amino acid modifications; (h) the heavy chain variable region comprises an amino acid sequence as set forth in any one of SEQ ID NOs: 1, 42, 43, 44, 45, 46, 47, 71, 72, or 93 with from 0 to 10 amino acid modifications, such as 1, 2, 3, 4, 5, 6, 7, 8, 9 or 10 amino acid modifications, and the light chain variable region comprises an amino acid sequence as set forth in SEQ ID NOs: 2, 48, 49, 50, 51, 52, 65, 66, or 94, with from 0 to 10 amino acid modifications, such as 1, 2, 3, 4, 5, 6, 7, 8, 9 or 10 amino acid modifications; or (i) the heavy chain variable region comprises an amino acid sequence as set forth in any one of SEQ ID NOs: 42, 43, 44, 45, 46, 47, 71, 72, or 93 with from 0 to 10 amino acid modifications, such as 1, 2, 3, 4, 5, 6, 7, 8, 9 or 10 amino acid modifications, and the light chain variable region comprises an amino acid sequence as set forth in SEQ ID NOs: 48, 49, 50, 51, 52, 65, 66, or 94 with from 0 to 10 amino acid modifications, such as 1, 2, 3, 4, 5, 6, 7, 8, 9 or 10 amino acid modifications.

Disclosed herein is an antibody or antigen-binding fragment thereof comprising a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 70, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some embodiments, the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 70, respectively, each with 0 to 2 amino acid modifications. In some embodiments, the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 70, respectively, each with 0 to 1 amino acid modifications. In some embodiments, the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 70, respectively. In some embodiments, the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 2 amino acid modifications. In some embodiments, the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 1 amino acid modifications. In some embodiments, the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively. In some embodiments, antibody or antigen-binding fragment thereof comprises an HCVR that comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 70, respectively, and an LCVR that comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively. In some embodiments, the HCVR comprises an amino acid sequence having at least 90%, 95%, 96%, 97%, 98%, 99%, or 100% sequence identity to the amino acid sequence as set forth in SEQ ID NO: 72. In some embodiments, the HCVR comprises an amino acid sequence having at least 95% sequence identity to the amino acid sequence as set forth in SEQ ID NO: 72. In some embodiments, the HCVR comprises an amino acid sequence having at least 97% sequence identity to the amino acid sequence as set forth in SEQ ID NO: 72. In some embodiments, the HCVR comprises an amino acid sequence having at least 98% sequence identity to the amino acid sequence as set forth in SEQ ID NO: 72. In some embodiments, the HCVR comprises an amino acid sequence having at least 99% sequence identity to the amino acid sequence as set forth in SEQ ID NO: 72. In some embodiments, the HCVR comprises an amino acid sequence having 100% sequence identity to the amino acid sequence as set forth in SEQ ID NO: 72. In some embodiments, the LCVR comprises an amino acid sequence having at least 90%, 95%, 96%, 97%, 98%, 99%, or 100% sequence identity to the amino acid sequence as set forth in SEQ ID NO: 65. In some embodiments, the LCVR comprises an amino acid sequence having at least 95% sequence identity to the amino acid sequence as set forth in SEQ ID NO: 65. In some embodiments, the LCVR comprises an amino acid sequence having at least 97% sequence identity to the amino acid sequence as set forth in SEQ ID NO: 65. In some embodiments, the LCVR comprises an amino acid sequence having at least 98% sequence identity to the amino acid sequence as set forth in SEQ ID NO: 65. In some embodiments, the LCVR comprises an amino acid sequence having at least 99% sequence identity to the amino acid sequence as set forth in SEQ ID NO: 65. In some embodiments, the LCVR comprises an amino acid sequence having 100% sequence identity to the amino acid sequence as set forth in SEQ ID NO: 65. In some embodiments, the HCVR comprises an amino acid sequence as set forth in SEQ ID NO: 72, with from 0 to 10 amino acid modifications, such as 1, 2, 3, 4, 5, 6, 7, 8, 9 or 10 amino acid modifications. In some embodiments, the HCVR comprises an amino acid sequence as set forth in SEQ ID NO: 72, with from 0 to 9 amino acid modifications. In some embodiments, the HCVR comprises an amino acid sequence as set forth in SEQ ID NO: 72, with from 0 to 8 amino acid modifications. In some embodiments, the HCVR comprises an amino acid sequence as set forth in SEQ ID NO: 72, with from 0 to 7 amino acid modifications. In some embodiments, the HCVR comprises an amino acid sequence as set forth in SEQ ID NO: 72, with from 0 to 6 amino acid modifications. In some embodiments, the HCVR comprises an amino acid sequence as set forth in SEQ ID NO: 72, with from 0 to 5 amino acid modifications. In some embodiments, the HCVR comprises an amino acid sequence as set forth in SEQ ID NO: 72, with from 0 to 4 amino acid modifications. In some embodiments, the HCVR comprises an amino acid sequence as set forth in SEQ ID NO: 72, with from 0 to 3 amino acid modifications. In some embodiments, the HCVR comprises an amino acid sequence as set forth in SEQ ID NO: 72, with from 0 to 2 amino acid modifications. In some embodiments, the HCVR comprises an amino acid sequence as set forth in SEQ ID NO: 72, with from 0 to 1 amino acid modifications. In some embodiments, the HCVR comprises an amino acid sequence as set forth in SEQ ID NO: 72. In some embodiments, the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 65, with from 0 to 9 amino acid modifications. In some embodiments, the LCVR comprises an amino acid sequence as set forth in SEQ ID NO: 65, with from 0 to 8 amino acid modifications. In some embodiments, the LCVR comprises an amino acid sequence as set forth in SEQ ID NO: 65, with from 0 to 7 amino acid modifications. In some embodiments, the LCVR comprises an amino acid sequence as set forth in SEQ ID NO: 65, with from 0 to 6 amino acid modifications. In some embodiments, the LCVR comprises an amino acid sequence as set forth in SEQ ID NO: 65, with from 0 to 5 amino acid modifications. In some embodiments, the LCVR comprises an amino acid sequence as set forth in SEQ ID NO: 65, with from 0 to 4 amino acid modifications. In some embodiments, the LCVR comprises an amino acid sequence as set forth in SEQ ID NO: 65, with from 0 to 3 amino acid modifications. In some embodiments, the LCVR comprises an amino acid sequence as set forth in SEQ ID NO: 65, with from 0 to 2 amino acid modifications. In some embodiments, the LCVR comprises an amino acid sequence as set forth in SEQ ID NO: 65, with from 0 to 1 amino acid modifications. In some embodiments, the LCVR comprises an amino acid sequence as set forth in SEQ ID NO: 65.

Disclosed herein is an antibody or antigen-binding fragment thereof comprising a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 5, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some embodiments, the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 5, respectively, each with 0 to 2 amino acid modifications. In some embodiments, the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 5, respectively, each with 0 to 1 amino acid modifications. In some embodiments, the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 5, respectively. In some embodiments, the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 2 amino acid modifications. In some embodiments, the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 1 amino acid modifications. In some embodiments, the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively. In some embodiments, antibody or antigen-binding fragment thereof comprises an HCVR that comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 5, respectively, and an LCVR that comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively. In some embodiments, the HCVR comprises an amino acid sequence having at least 90%, 95%, 96%, 97%, 98%, 99%, or 100% sequence identity to the amino acid sequence as set forth in SEQ ID NO: 71. In some embodiments, the HCVR comprises an amino acid sequence having at least 95% sequence identity to the amino acid sequence as set forth in SEQ ID NO: 71. In some embodiments, the HCVR comprises an amino acid sequence having at least 97% sequence identity to the amino acid sequence as set forth in SEQ ID NO: 71. In some embodiments, the HCVR comprises an amino acid sequence having at least 98% sequence identity to the amino acid sequence as set forth in SEQ ID NO: 71. In some embodiments, the HCVR comprises an amino acid sequence having at least 99% sequence identity to the amino acid sequence as set forth in SEQ ID NO: 71. In some embodiments, the HCVR comprises an amino acid sequence having 100% sequence identity to the amino acid sequence as set forth in SEQ ID NO: 71. In some embodiments, the LCVR comprises an amino acid sequence having at least 90%, 95%, 96%, 97%, 98%, 99%, or 100% sequence identity to the amino acid sequence as set forth in SEQ ID NO: 65. In some embodiments, the LCVR comprises an amino acid sequence having at least 95% sequence identity to the amino acid sequence as set forth in SEQ ID NO: 65. In some embodiments, the LCVR comprises an amino acid sequence having at least 97% sequence identity to the amino acid sequence as set forth in SEQ ID NO: 65. In some embodiments, the LCVR comprises an amino acid sequence having at least 98% sequence identity to the amino acid sequence as set forth in SEQ ID NO: 65. In some embodiments, the LCVR comprises an amino acid sequence having at least 99% sequence identity to the amino acid sequence as set forth in SEQ ID NO: 65. In some embodiments, the LCVR comprises an amino acid sequence having 100% sequence identity to the amino acid sequence as set forth in SEQ ID NO: 65. In some embodiments, the HCVR comprises an amino acid sequence as set forth in SEQ ID NO: 71, with from 0 to 10 amino acid modifications, such as 1, 2, 3, 4, 5, 6, 7, 8, 9 or 10 amino acid modifications. In some embodiments, the HCVR comprises an amino acid sequence as set forth in SEQ ID NO: 71, with from 0 to 9 amino acid modifications. In some embodiments, the HCVR comprises an amino acid sequence as set forth in SEQ ID NO: 71, with from 0 to 8 amino acid modifications. In some embodiments, the HCVR comprises an amino acid sequence as set forth in SEQ ID NO: 71, with from 0 to 7 amino acid modifications. In some embodiments, the HCVR comprises an amino acid sequence as set forth in SEQ ID NO: 71, with from 0 to 6 amino acid modifications. In some embodiments, the HCVR comprises an amino acid sequence as set forth in SEQ ID NO: 71, with from 0 to 5 amino acid modifications. In some embodiments, the HCVR comprises an amino acid sequence as set forth in SEQ ID NO: 71, with from 0 to 4 amino acid modifications. In some embodiments, the HCVR comprises an amino acid sequence as set forth in SEQ ID NO: 71, with from 0 to 3 amino acid modifications. In some embodiments, the HCVR comprises an amino acid sequence as set forth in SEQ ID NO: 71, with from 0 to 2 amino acid modifications. In some embodiments, the HCVR comprises an amino acid sequence as set forth in SEQ ID NO: 71, with from 0 to 1 amino acid modifications. In some embodiments, the HCVR comprises an amino acid sequence as set forth in SEQ ID NO: 71. In some embodiments, the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 65, with from 0 to 9 amino acid modifications. In some embodiments, the LCVR comprises an amino acid sequence as set forth in SEQ ID NO: 65, with from 0 to 8 amino acid modifications. In some embodiments, the LCVR comprises an amino acid sequence as set forth in SEQ ID NO: 65, with from 0 to 7 amino acid modifications. In some embodiments, the LCVR comprises an amino acid sequence as set forth in SEQ ID NO: 65, with from 0 to 6 amino acid modifications. In some embodiments, the LCVR comprises an amino acid sequence as set forth in SEQ ID NO: 65, with from 0 to 5 amino acid modifications. In some embodiments, the LCVR comprises an amino acid sequence as set forth in SEQ ID NO: 65, with from 0 to 4 amino acid modifications. In some embodiments, the LCVR comprises an amino acid sequence as set forth in SEQ ID NO: 65, with from 0 to 3 amino acid modifications. In some embodiments, the LCVR comprises an amino acid sequence as set forth in SEQ ID NO: 65, with from 0 to 2 amino acid modifications. In some embodiments, the LCVR comprises an amino acid sequence as set forth in SEQ ID NO: 65, with from 0 to 1 amino acid modifications. In some embodiments, the LCVR comprises an amino acid sequence as set forth in SEQ ID NO: 65.

Disclosed herein is an antibody or antigen-binding fragment thereof comprising (a) a heavy chain that comprises a heavy chain variable region (HCVR), wherein the HCVR comprises 1 to 3 heavy chain CDRs as set forth in any of SEQ ID NO: 3, 4, 41, 5, 69, or 70, with from 0 to 3 amino acid modifications; and (b) a light chain that comprises a light chain variable region (LCVR), wherein the LCVR comprises 1 to 3 light chain CDRs as set forth in any of SEQ ID NO: 6, 67, or 8, with from 0 to 3 amino acid modifications.

Disclosed herein is an antibody or antigen-binding fragment thereof comprising (a) a heavy chain that comprises a heavy chain variable region (HCVR), wherein the HCVR comprises 1 to 3 heavy chain CDRs as set forth in any of SEQ ID NO: 3, 4, 41, 5, 69, or 70, with from 0 to 3 amino acid modifications; and (b) a light chain that comprises a light chain variable region (LCVR), wherein the LCVR comprises 1 to 3 light chain CDRs as set forth in any of SEQ ID NO: 6, 7, or 8, with from 0 to 3 amino acid modifications.

Disclosed herein is an antibody or antigen-binding fragment thereof comprising (a) a heavy chain that comprises a heavy chain variable region (HCVR), wherein the HCVR comprises 1 to 3 heavy chain CDRs as set forth in any of SEQ ID NO: 3, 4, 41, 5, 69, or 70, with from 0 to 3 amino acid modifications; and (b) a light chain that comprises a light chain variable region (LCVR), wherein the LCVR comprises 1 to 3 light chain CDRs as set forth in any of SEQ ID NO: 6, 68, or 8, with from 0 to 3 amino acid modifications.

Disclosed herein is an antibody or antigen-binding fragment thereof comprising (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 4, 5, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 7, 8, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 1. In some embodiments, the HCVR comprises an amino acid sequence having at least 95% sequence identity to the amino acid sequence as set forth in SEQ ID NO: 1. In some embodiments, the HCVR comprises an amino acid sequence having at least 97% sequence identity to the amino acid sequence as set forth in SEQ ID NO: 1. In some embodiments, the HCVR comprises an amino acid sequence having at least 98% sequence identity to the amino acid sequence as set forth in SEQ ID NO: 1. In some embodiments, the HCVR comprises an amino acid sequence having at least 99% sequence identity to the amino acid sequence as set forth in SEQ ID NO: 1. In some embodiments, the HCVR comprises an amino acid sequence having 100% sequence identity to the amino acid sequence as set forth in SEQ ID NO: 1. In some embodiments, the LCVR comprises an amino acid sequence having at least 90%, 95%, 96%, 97%, 98%, 99%, or 100% sequence identity to the amino acid sequence as set forth in SEQ ID NO: 2. In some embodiments, the LCVR comprises an amino acid sequence having at least 95% sequence identity to the amino acid sequence as set forth in SEQ ID NO: 2. In some embodiments, the LCVR comprises an amino acid sequence having at least 97% sequence identity to the amino acid sequence as set forth in SEQ ID NO: 2. In some embodiments, the LCVR comprises an amino acid sequence having at least 98% sequence identity to the amino acid sequence as set forth in SEQ ID NO: 2. In some embodiments, the LCVR comprises an amino acid sequence having at least 99% sequence identity to the amino acid sequence as set forth in SEQ ID NO: 2. In some embodiments, the LCVR comprises an amino acid sequence having 100% sequence identity to the amino acid sequence as set forth in SEQ ID NO: 2. In some embodiments, the HCVR comprises an amino acid sequence as set forth in SEQ ID NO: 1, with from 0 to 10 amino acid modifications, such as 1, 2, 3, 4, 5, 6, 7, 8, 9 or 10 amino acid modifications. In some embodiments, the HCVR comprises an amino acid sequence as set forth in SEQ ID NO: 1, with from 0 to 9 amino acid modifications. In some embodiments, the HCVR comprises an amino acid sequence as set forth in SEQ ID NO: 1, with from 0 to 8 amino acid modifications. In some embodiments, the HCVR comprises an amino acid sequence as set forth in SEQ ID NO: 1, with from 0 to 7 amino acid modifications. In some embodiments, the HCVR comprises an amino acid sequence as set forth in SEQ ID NO: 1, with from 0 to 6 amino acid modifications. In some embodiments, the HCVR comprises an amino acid sequence as set forth in SEQ ID NO: 1, with from 0 to 5 amino acid modifications. In some embodiments, the HCVR comprises an amino acid sequence as set forth in SEQ ID NO: 1, with from 0 to 4 amino acid modifications. In some embodiments, the HCVR comprises an amino acid sequence as set forth in SEQ ID NO: 1, with from 0 to 3 amino acid modifications. In some embodiments, the HCVR comprises an amino acid sequence as set forth in SEQ ID NO: 1, with from 0 to 2 amino acid modifications. In some embodiments, the HCVR comprises an amino acid sequence as set forth in SEQ ID NO: 1, with from 0 to 1 amino acid modifications. In some embodiments, the HCVR comprises an amino acid sequence as set forth in SEQ ID NO: 1. In some embodiments, the LCVR comprises an amino acid sequence as set forth in SEQ ID NO: 2, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 2, with from 0 to 9 amino acid modifications. In some embodiments, the LCVR comprises an amino acid sequence as set forth in SEQ ID NO: 2, with from 0 to 8 amino acid modifications. In some embodiments, the LCVR comprises an amino acid sequence as set forth in SEQ ID NO: 2, with from 0 to 7 amino acid modifications. In some embodiments, the LCVR comprises an amino acid sequence as set forth in SEQ ID NO: 2, with from 0 to 6 amino acid modifications. In some embodiments, the LCVR comprises an amino acid sequence as set forth in SEQ ID NO: 2, with from 0 to 5 amino acid modifications. In some embodiments, the LCVR comprises an amino acid sequence as set forth in SEQ ID NO: 2, with from 0 to 4 amino acid modifications. In some embodiments, the LCVR comprises an amino acid sequence as set forth in SEQ ID NO: 2, with from 0 to 3 amino acid modifications. In some embodiments, the LCVR comprises an amino acid sequence as set forth in SEQ ID NO: 2, with from 0 to 2 amino acid modifications. In some embodiments, the LCVR comprises an amino acid sequence as set forth in SEQ ID NO: 2, with from 0 to 1 amino acid modifications. In some embodiments, the LCVR comprises an amino acid sequence as set forth in SEQ ID NO: 2.

Disclosed herein is an antibody or antigen-binding fragment thereof comprising (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 11, 12, or 13, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 14, 15, or 16, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 9. In some embodiments, the HCVR comprises an amino acid sequence having at least 95% sequence identity to the amino acid sequence as set forth in SEQ ID NO: 9. In some embodiments, the HCVR comprises an amino acid sequence having at least 97% sequence identity to the amino acid sequence as set forth in SEQ ID NO: 9. In some embodiments, the HCVR comprises an amino acid sequence having at least 98% sequence identity to the amino acid sequence as set forth in SEQ ID NO: 9. In some embodiments, the HCVR comprises an amino acid sequence having at least 99% sequence identity to the amino acid sequence as set forth in SEQ ID NO: 9. In some embodiments, the HCVR comprises an amino acid sequence having 100% sequence identity to the amino acid sequence as set forth in SEQ ID NO: 9. In some embodiments, the LCVR comprises an amino acid sequence having at least 90%, 95%, 96%, 97%, 98%, 99%, or 100% sequence identity to the amino acid sequence as set forth in SEQ ID NO: 10. In some embodiments, the LCVR comprises an amino acid sequence having at least 95% sequence identity to the amino acid sequence as set forth in SEQ ID NO: 10. In some embodiments, the LCVR comprises an amino acid sequence having at least 97% sequence identity to the amino acid sequence as set forth in SEQ ID NO: 10. In some embodiments, the LCVR comprises an amino acid sequence having at least 98% sequence identity to the amino acid sequence as set forth in SEQ ID NO: 10. In some embodiments, the LCVR comprises an amino acid sequence having at least 99% sequence identity to the amino acid sequence as set forth in SEQ ID NO: 10. In some embodiments, the LCVR comprises an amino acid sequence having 100% sequence identity to the amino acid sequence as set forth in SEQ ID NO: 10. In some embodiments, the HCVR comprises an amino acid sequence as set forth in SEQ ID NO: 9, with from 0 to 10 amino acid modifications, such as 1, 2, 3, 4, 5, 6, 7, 8, 9 or 10 amino acid modifications. In some embodiments, the HCVR comprises an amino acid sequence as set forth in SEQ ID NO: 9, with from 0 to 9 amino acid modifications. In some embodiments, the HCVR comprises an amino acid sequence as set forth in SEQ ID NO: 9, with from 0 to 8 amino acid modifications. In some embodiments, the HCVR comprises an amino acid sequence as set forth in SEQ ID NO: 9, with from 0 to 7 amino acid modifications. In some embodiments, the HCVR comprises an amino acid sequence as set forth in SEQ ID NO: 9, with from 0 to 6 amino acid modifications. In some embodiments, the HCVR comprises an amino acid sequence as set forth in SEQ ID NO: 9, with from 0 to 5 amino acid modifications. In some embodiments, the HCVR comprises an amino acid sequence as set forth in SEQ ID NO: 9, with from 0 to 4 amino acid modifications. In some embodiments, the HCVR comprises an amino acid sequence as set forth in SEQ ID NO: 9, with from 0 to 3 amino acid modifications. In some embodiments, the HCVR comprises an amino acid sequence as set forth in SEQ ID NO: 9, with from 0 to 2 amino acid modifications. In some embodiments, the HCVR comprises an amino acid sequence as set forth in SEQ ID NO: 9, with from 0 to 1 amino acid modifications. In some embodiments, the HCVR comprises an amino acid sequence as set forth in SEQ ID NO: 9. In some embodiments, the LCVR comprises an amino acid sequence as set forth in SEQ ID NO: 10, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 10, with from 0 to 9 amino acid modifications. In some embodiments, the LCVR comprises an amino acid sequence as set forth in SEQ ID NO: 10, with from 0 to 8 amino acid modifications. In some embodiments, the LCVR comprises an amino acid sequence as set forth in SEQ ID NO: 10, with from 0 to 7 amino acid modifications. In some embodiments, the LCVR comprises an amino acid sequence as set forth in SEQ ID NO: 10, with from 0 to 6 amino acid modifications. In some embodiments, the LCVR comprises an amino acid sequence as set forth in SEQ ID NO: 10, with from 0 to 5 amino acid modifications. In some embodiments, the LCVR comprises an amino acid sequence as set forth in SEQ ID NO: 10, with from 0 to 4 amino acid modifications. In some embodiments, the LCVR comprises an amino acid sequence as set forth in SEQ ID NO: 10, with from 0 to 3 amino acid modifications. In some embodiments, the LCVR comprises an amino acid sequence as set forth in SEQ ID NO: 10, with from 0 to 2 amino acid modifications. In some embodiments, the LCVR comprises an amino acid sequence as set forth in SEQ ID NO: 10, with from 0 to 1 amino acid modifications. In some embodiments, the LCVR comprises an amino acid sequence as set forth in SEQ ID NO: 10.

Disclosed herein is an antibody or antigen-binding fragment thereof comprising (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 19, 20, or 21, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 22, 23, or 24, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 17. In some embodiments, the HCVR comprises an amino acid sequence having at least 95% sequence identity to the amino acid sequence as set forth in SEQ ID NO: 17. In some embodiments, the HCVR comprises an amino acid sequence having at least 97% sequence identity to the amino acid sequence as set forth in SEQ ID NO: 17. In some embodiments, the HCVR comprises an amino acid sequence having at least 98% sequence identity to the amino acid sequence as set forth in SEQ ID NO: 17. In some embodiments, the HCVR comprises an amino acid sequence having at least 99% sequence identity to the amino acid sequence as set forth in SEQ ID NO: 17. In some embodiments, the HCVR comprises an amino acid sequence having 100% sequence identity to the amino acid sequence as set forth in SEQ ID NO: 17. In some embodiments, the LCVR comprises an amino acid sequence having at least 90%, 95%, 96%, 97%, 98%, 99%, or 100% sequence identity to the amino acid sequence as set forth in SEQ ID NO: 18. In some embodiments, the LCVR comprises an amino acid sequence having at least 95% sequence identity to the amino acid sequence as set forth in SEQ ID NO: 18. In some embodiments, the LCVR comprises an amino acid sequence having at least 97% sequence identity to the amino acid sequence as set forth in SEQ ID NO: 18. In some embodiments, the LCVR comprises an amino acid sequence having at least 98% sequence identity to the amino acid sequence as set forth in SEQ ID NO: 18. In some embodiments, the LCVR comprises an amino acid sequence having at least 99% sequence identity to the amino acid sequence as set forth in SEQ ID NO: 18. In some embodiments, the LCVR comprises an amino acid sequence having 100% sequence identity to the amino acid sequence as set forth in SEQ ID NO: 18. In some embodiments, the HCVR comprises an amino acid sequence as set forth in SEQ ID NO: 17, with from 0 to 10 amino acid modifications, such as 1, 2, 3, 4, 5, 6, 7, 8, 9 or 10 amino acid modifications. In some embodiments, the HCVR comprises an amino acid sequence as set forth in SEQ ID NO: 17, with from 0 to 9 amino acid modifications. In some embodiments, the HCVR comprises an amino acid sequence as set forth in SEQ ID NO: 17, with from 0 to 8 amino acid modifications. In some embodiments, the HCVR comprises an amino acid sequence as set forth in SEQ ID NO: 17, with from 0 to 7 amino acid modifications. In some embodiments, the HCVR comprises an amino acid sequence as set forth in SEQ ID NO: 17, with from 0 to 6 amino acid modifications. In some embodiments, the HCVR comprises an amino acid sequence as set forth in SEQ ID NO: 17, with from 0 to 5 amino acid modifications. In some embodiments, the HCVR comprises an amino acid sequence as set forth in SEQ ID NO: 17, with from 0 to 4 amino acid modifications. In some embodiments, the HCVR comprises an amino acid sequence as set forth in SEQ ID NO: 17, with from 0 to 3 amino acid modifications. In some embodiments, the HCVR comprises an amino acid sequence as set forth in SEQ ID NO: 17, with from 0 to 2 amino acid modifications. In some embodiments, the HCVR comprises an amino acid sequence as set forth in SEQ ID NO: 17, with from 0 to 1 amino acid modifications. In some embodiments, the HCVR comprises an amino acid sequence as set forth in SEQ ID NO: 17. In some embodiments, the LCVR comprises an amino acid sequence as set forth in SEQ ID NO: 18, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 18, with from 0 to 9 amino acid modifications. In some embodiments, the LCVR comprises an amino acid sequence as set forth in SEQ ID NO: 18, with from 0 to 8 amino acid modifications. In some embodiments, the LCVR comprises an amino acid sequence as set forth in SEQ ID NO: 18, with from 0 to 7 amino acid modifications. In some embodiments, the LCVR comprises an amino acid sequence as set forth in SEQ ID NO: 18, with from 0 to 6 amino acid modifications. In some embodiments, the LCVR comprises an amino acid sequence as set forth in SEQ ID NO: 18, with from 0 to 5 amino acid modifications. In some embodiments, the LCVR comprises an amino acid sequence as set forth in SEQ ID NO: 18, with from 0 to 4 amino acid modifications. In some embodiments, the LCVR comprises an amino acid sequence as set forth in SEQ ID NO: 18, with from 0 to 3 amino acid modifications. In some embodiments, the LCVR comprises an amino acid sequence as set forth in SEQ ID NO: 18, with from 0 to 2 amino acid modifications. In some embodiments, the LCVR comprises an amino acid sequence as set forth in SEQ ID NO: 18, with from 0 to 1 amino acid modifications. In some embodiments, the LCVR comprises an amino acid sequence as set forth in SEQ ID NO: 18.

Disclosed herein is an antibody or antigen-binding fragment thereof comprising (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 27, 28, or 29, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 30, 31, or 32, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 25. In some embodiments, the HCVR comprises an amino acid sequence having at least 95% sequence identity to the amino acid sequence as set forth in SEQ ID NO: 25. In some embodiments, the HCVR comprises an amino acid sequence having at least 97% sequence identity to the amino acid sequence as set forth in SEQ ID NO: 25. In some embodiments, the HCVR comprises an amino acid sequence having at least 98% sequence identity to the amino acid sequence as set forth in SEQ ID NO: 25. In some embodiments, the HCVR comprises an amino acid sequence having at least 99% sequence identity to the amino acid sequence as set forth in SEQ ID NO: 25. In some embodiments, the HCVR comprises an amino acid sequence having 100% sequence identity to the amino acid sequence as set forth in SEQ ID NO: 25. In some embodiments, the LCVR comprises an amino acid sequence having at least 90%, 95%, 96%, 97%, 98%, 99%, or 100% sequence identity to the amino acid sequence as set forth in SEQ ID NO: 26. In some embodiments, the LCVR comprises an amino acid sequence having at least 95% sequence identity to the amino acid sequence as set forth in SEQ ID NO: 26. In some embodiments, the LCVR comprises an amino acid sequence having at least 97% sequence identity to the amino acid sequence as set forth in SEQ ID NO: 26. In some embodiments, the LCVR comprises an amino acid sequence having at least 98% sequence identity to the amino acid sequence as set forth in SEQ ID NO: 26. In some embodiments, the LCVR comprises an amino acid sequence having at least 99% sequence identity to the amino acid sequence as set forth in SEQ ID NO: 26. In some embodiments, the LCVR comprises an amino acid sequence having 100% sequence identity to the amino acid sequence as set forth in SEQ ID NO: 26. In some embodiments, the HCVR comprises an amino acid sequence as set forth in SEQ ID NO: 25, with from 0 to 10 amino acid modifications, such as 1, 2, 3, 4, 5, 6, 7, 8, 9 or 10 amino acid modifications. In some embodiments, the HCVR comprises an amino acid sequence as set forth in SEQ ID NO: 25, with from 0 to 9 amino acid modifications. In some embodiments, the HCVR comprises an amino acid sequence as set forth in SEQ ID NO: 25, with from 0 to 8 amino acid modifications. In some embodiments, the HCVR comprises an amino acid sequence as set forth in SEQ ID NO: 25, with from 0 to 7 amino acid modifications. In some embodiments, the HCVR comprises an amino acid sequence as set forth in SEQ ID NO: 25, with from 0 to 6 amino acid modifications. In some embodiments, the HCVR comprises an amino acid sequence as set forth in SEQ ID NO: 25, with from 0 to 5 amino acid modifications. In some embodiments, the HCVR comprises an amino acid sequence as set forth in SEQ ID NO: 25, with from 0 to 4 amino acid modifications. In some embodiments, the HCVR comprises an amino acid sequence as set forth in SEQ ID NO: 25, with from 0 to 3 amino acid modifications. In some embodiments, the HCVR comprises an amino acid sequence as set forth in SEQ ID NO: 25, with from 0 to 2 amino acid modifications. In some embodiments, the HCVR comprises an amino acid sequence as set forth in SEQ ID NO: 25, with from 0 to 1 amino acid modifications. In some embodiments, the HCVR comprises an amino acid sequence as set forth in SEQ ID NO: 25. In some embodiments, the LCVR comprises an amino acid sequence as set forth in SEQ ID NO: 26, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 26, with from 0 to 9 amino acid modifications. In some embodiments, the LCVR comprises an amino acid sequence as set forth in SEQ ID NO: 26, with from 0 to 8 amino acid modifications. In some embodiments, the LCVR comprises an amino acid sequence as set forth in SEQ ID NO: 26, with from 0 to 7 amino acid modifications. In some embodiments, the LCVR comprises an amino acid sequence as set forth in SEQ ID NO: 26, with from 0 to 6 amino acid modifications. In some embodiments, the LCVR comprises an amino acid sequence as set forth in SEQ ID NO: 26, with from 0 to 5 amino acid modifications. In some embodiments, the LCVR comprises an amino acid sequence as set forth in SEQ ID NO: 26, with from 0 to 4 amino acid modifications. In some embodiments, the LCVR comprises an amino acid sequence as set forth in SEQ ID NO: 26, with from 0 to 3 amino acid modifications. In some embodiments, the LCVR comprises an amino acid sequence as set forth in SEQ ID NO: 26, with from 0 to 2 amino acid modifications. In some embodiments, the LCVR comprises an amino acid sequence as set forth in SEQ ID NO: 26, with from 0 to 1 amino acid modifications. In some embodiments, the LCVR comprises an amino acid sequence as set forth in SEQ ID NO: 26.

Disclosed herein is an antibody or antigen-binding fragment thereof comprising (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 35, 36, or 37, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 38, 39, or 40, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 33. In some embodiments, the HCVR comprises an amino acid sequence having at least 95% sequence identity to the amino acid sequence as set forth in SEQ ID NO: 33. In some embodiments, the HCVR comprises an amino acid sequence having at least 97% sequence identity to the amino acid sequence as set forth in SEQ ID NO: 33. In some embodiments, the HCVR comprises an amino acid sequence having at least 98% sequence identity to the amino acid sequence as set forth in SEQ ID NO: 33. In some embodiments, the HCVR comprises an amino acid sequence having at least 99% sequence identity to the amino acid sequence as set forth in SEQ ID NO: 33. In some embodiments, the HCVR comprises an amino acid sequence having 100% sequence identity to the amino acid sequence as set forth in SEQ ID NO: 33. In some embodiments, the LCVR comprises an amino acid sequence having at least 90%, 95%, 96%, 97%, 98%, 99%, or 100% sequence identity to the amino acid sequence as set forth in SEQ ID NO: 34. In some embodiments, the LCVR comprises an amino acid sequence having at least 95% sequence identity to the amino acid sequence as set forth in SEQ ID NO: 34. In some embodiments, the LCVR comprises an amino acid sequence having at least 97% sequence identity to the amino acid sequence as set forth in SEQ ID NO: 34. In some embodiments, the LCVR comprises an amino acid sequence having at least 98% sequence identity to the amino acid sequence as set forth in SEQ ID NO: 34. In some embodiments, the LCVR comprises an amino acid sequence having at least 99% sequence identity to the amino acid sequence as set forth in SEQ ID NO: 34. In some embodiments, the LCVR comprises an amino acid sequence having 100% sequence identity to the amino acid sequence as set forth in SEQ ID NO: 34. In some embodiments, the HCVR comprises an amino acid sequence as set forth in SEQ ID NO: 33, with from 0 to 10 amino acid modifications, such as 1, 2, 3, 4, 5, 6, 7, 8, 9 or 10 amino acid modifications. In some embodiments, the HCVR comprises an amino acid sequence as set forth in SEQ ID NO: 33, with from 0 to 9 amino acid modifications. In some embodiments, the HCVR comprises an amino acid sequence as set forth in SEQ ID NO: 33, with from 0 to 8 amino acid modifications. In some embodiments, the HCVR comprises an amino acid sequence as set forth in SEQ ID NO: 33, with from 0 to 7 amino acid modifications. In some embodiments, the HCVR comprises an amino acid sequence as set forth in SEQ ID NO: 33, with from 0 to 6 amino acid modifications. In some embodiments, the HCVR comprises an amino acid sequence as set forth in SEQ ID NO: 33, with from 0 to 5 amino acid modifications. In some embodiments, the HCVR comprises an amino acid sequence as set forth in SEQ ID NO: 33, with from 0 to 4 amino acid modifications. In some embodiments, the HCVR comprises an amino acid sequence as set forth in SEQ ID NO: 33, with from 0 to 3 amino acid modifications. In some embodiments, the HCVR comprises an amino acid sequence as set forth in SEQ ID NO: 33, with from 0 to 2 amino acid modifications. In some embodiments, the HCVR comprises an amino acid sequence as set forth in SEQ ID NO: 33, with from 0 to 1 amino acid modifications. In some embodiments, the HCVR comprises an amino acid sequence as set forth in SEQ ID NO: 33. In some embodiments, the LCVR comprises an amino acid sequence as set forth in SEQ ID NO: 34, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 34, with from 0 to 9 amino acid modifications. In some embodiments, the LCVR comprises an amino acid sequence as set forth in SEQ ID NO: 34, with from 0 to 8 amino acid modifications. In some embodiments, the LCVR comprises an amino acid sequence as set forth in SEQ ID NO: 34, with from 0 to 7 amino acid modifications. In some embodiments, the LCVR comprises an amino acid sequence as set forth in SEQ ID NO: 34, with from 0 to 6 amino acid modifications. In some embodiments, the LCVR comprises an amino acid sequence as set forth in SEQ ID NO: 34, with from 0 to 5 amino acid modifications. In some embodiments, the LCVR comprises an amino acid sequence as set forth in SEQ ID NO: 34, with from 0 to 4 amino acid modifications. In some embodiments, the LCVR comprises an amino acid sequence as set forth in SEQ ID NO: 34, with from 0 to 3 amino acid modifications. In some embodiments, the LCVR comprises an amino acid sequence as set forth in SEQ ID NO: 34, with from 0 to 2 amino acid modifications. In some embodiments, the LCVR comprises an amino acid sequence as set forth in SEQ ID NO: 34, with from 0 to 1 amino acid modifications. In some embodiments, the LCVR comprises an amino acid sequence as set forth in SEQ ID NO: 34.

In some embodiments, the LCVR is linked to a light chain constant region comprising an amino acid sequence having at least 90%, 95%, 96%, 97%, 98%, 99%, or 100% sequence identity to the amino acid sequence as set forth in SEQ ID NO: 62. In some embodiments, the LCVR is linked to a light chain constant region comprising an amino acid sequence having at least 95% sequence identity to the amino acid sequence as set forth in SEQ ID NO: 62. In some embodiments, the LCVR is linked to a light chain constant region comprising an amino acid sequence having at least 97% sequence identity to the amino acid sequence as set forth in SEQ ID NO: 62. In some embodiments, the LCVR is linked to a light chain constant region comprising an amino acid sequence having at least 98% sequence identity to the amino acid sequence as set forth in SEQ ID NO: 62. In some embodiments, the LCVR is linked to a light chain constant region comprising an amino acid sequence having at least 99% sequence identity to the amino acid sequence as set forth in SEQ ID NO: 62. In some embodiments, the LCVR is linked to a light chain constant region comprising an amino acid sequence having 100% sequence identity to the amino acid sequence as set forth in SEQ ID NO: 62.

In some embodiments, the light chain variable region is linked to a light chain constant region comprising an amino acid sequence as set forth in SEQ ID NO: 62, with from 0 to 10 amino acid modifications, such as 1, 2, 3, 4, 5, 6, 7, 8, 9 or 10 amino acid modifications. In some embodiments, the light chain variable region is linked to a light chain constant region comprising an amino acid sequence as set forth in SEQ ID NO: 62, with from 0 to 9 amino acid modifications. In some embodiments, the light chain variable region is linked to a light chain constant region comprising an amino acid sequence as set forth in SEQ ID NO: 62, with from 0 to 8 amino acid modifications. In some embodiments, the light chain variable region is linked to a light chain constant region comprising an amino acid sequence as set forth in SEQ ID NO: 62, with from 0 to 7 amino acid modifications. In some embodiments, the light chain variable region is linked to a light chain constant region comprising an amino acid sequence as set forth in SEQ ID NO: 62, with from 0 to 6 amino acid modifications. In some embodiments, the light chain variable region is linked to a light chain constant region comprising an amino acid sequence as set forth in SEQ ID NO: 62, with from 0 to 5 amino acid modifications. In some embodiments, the light chain variable region is linked to a light chain constant region comprising an amino acid sequence as set forth in SEQ ID NO: 62, with from 0 to 4 amino acid modifications. In some embodiments, the light chain variable region is linked to a light chain constant region comprising an amino acid sequence as set forth in SEQ ID NO: 62, with from 0 to 3 amino acid modifications. In some embodiments, the light chain variable region is linked to a light chain constant region comprising an amino acid sequence as set forth in SEQ ID NO: 62, with from 0 to 2 amino acid modifications. In some embodiments, the light chain variable region is linked to a light chain constant region comprising an amino acid sequence as set forth in SEQ ID NO: 62, with from 0 to 1 amino acid modifications. In some embodiments, the light chain variable region is linked to a light chain constant region comprising an amino acid sequence as set forth in SEQ ID NO: 62.

In some embodiments, the light chain variable region is linked to a light chain constant region comprising an amino acid sequence having at least 90%, 95%, 96%, 97%, 98%, 99%, or 100% sequence identity to the amino acid sequence as set forth in SEQ ID NO: 64. In some embodiments, the light chain variable region is linked to a light chain constant region comprising an amino acid sequence having at least 95% sequence identity to the amino acid sequence as set forth in SEQ ID NO: 64. In some embodiments, the light chain variable region is linked to a light chain constant region comprising an amino acid sequence having at least 97% sequence identity to the amino acid sequence as set forth in SEQ ID NO: 64. In some embodiments, the light chain variable region is linked to a light chain constant region comprising an amino acid sequence having at least 98% sequence identity to the amino acid sequence as set forth in SEQ ID NO: 64. In some embodiments, the light chain variable region is linked to a light chain constant region comprising an amino acid sequence having at least 99% sequence identity to the amino acid sequence as set forth in SEQ ID NO: 64. In some embodiments, the light chain variable region is linked to a light chain constant region comprising an amino acid sequence having 100% sequence identity to the amino acid sequence as set forth in SEQ ID NO: 64.

In some embodiments, the light chain variable region is linked to a light chain constant region comprising an amino acid sequence as set forth in SEQ ID NO: 64, with from 0 to 10 amino acid modifications, such as 1, 2, 3, 4, 5, 6, 7, 8, 9 or 10 amino acid modifications. In some embodiments, the light chain variable region is linked to a light chain constant region comprising an amino acid sequence as set forth in SEQ ID NO: 64, with from 0 to 9 amino acid modifications. In some embodiments, the light chain variable region is linked to a light chain constant region comprising an amino acid sequence as set forth in SEQ ID NO: 64, with from 0 to 8 amino acid modifications. In some embodiments, the light chain variable region is linked to a light chain constant region comprising an amino acid sequence as set forth in SEQ ID NO: 64, with from 0 to 7 amino acid modifications. In some embodiments, the light chain variable region is linked to a light chain constant region comprising an amino acid sequence as set forth in SEQ ID NO: 64, with from 0 to 6amino acid modifications. In some embodiments, the light chain variable region is linked to a light chain constant region comprising an amino acid sequence as set forth in SEQ ID NO: 64, with from 0 to 5 amino acid modifications. In some embodiments, the light chain variable region is linked to a light chain constant region comprising an amino acid sequence as set forth in SEQ ID NO: 64, with from 0 to 4 amino acid modifications. In some embodiments, the light chain variable region is linked to a light chain constant region comprising an amino acid sequence as set forth in SEQ ID NO: 64, with from 0 to 3 amino acid modifications. In some embodiments, the light chain variable region is linked to a light chain constant region comprising an amino acid sequence as set forth in SEQ ID NO: 64, with from 0 to 2 amino acid modifications. In some embodiments, the light chain variable region is linked to a light chain constant region comprising an amino acid sequence as set forth in SEQ ID NO: 64, with from 0 to 1 amino acid modifications. In some embodiments, the light chain variable region is linked to a light chain constant region comprising an amino acid sequence as set forth in SEQ ID NO: 64.

In some embodiments, the heavy chain constant region comprises an amino acid sequence having at least 90% sequence identity to the amino acid sequence as set forth in SEQ ID NO: 61. In some embodiments, the heavy chain constant region comprises an amino acid sequence having at least 95% sequence identity to the amino acid sequence as set forth in SEQ ID NO: 61. In some embodiments, the heavy chain constant region comprises an amino acid sequence having at least 97% sequence identity to the amino acid sequence as set forth in SEQ ID NO: 61. In some embodiments, the heavy chain constant region comprises an amino acid sequence having at least 98% sequence identity to the amino acid sequence as set forth in SEQ ID NO: 61. In some embodiments, the heavy chain constant region comprises an amino acid sequence having at least 99% sequence identity to the amino acid sequence as set forth in SEQ ID NO: 61. In some embodiments, the heavy chain constant region comprises an amino acid sequence having 100% sequence identity to the amino acid sequence as set forth in SEQ ID NO: 61. In some embodiments, the HCVR is linked to a heavy chain constant region comprising an amino acid sequence as set forth in SEQ ID NO: 61, with from 0 to 10 amino acid modifications, such as 1, 2, 3, 4, 5, 6, 7, 8, 9 or 10 amino acid modifications. In some embodiments, the heavy chain constant region comprises an amino acid sequence as set forth in SEQ ID NO: 61, with from 0 to 9 amino acid modifications. In some embodiments, the heavy chain constant region comprises an amino acid sequence as set forth in SEQ ID NO: 61, with from 0 to 8 amino acid modifications. In some embodiments, the heavy chain constant region comprises an amino acid sequence as set forth in SEQ ID NO: 61, with from 0 to 7 amino acid modifications. In some embodiments, the heavy chain constant region comprises an amino acid sequence as set forth in SEQ ID NO: 61, with from 0 to 6 amino acid modifications. In some embodiments, the heavy chain constant region comprises an amino acid sequence as set forth in SEQ ID NO: 61, with from 0 to 5 amino acid modifications. In some embodiments, the heavy chain constant region comprises an amino acid sequence as set forth in SEQ ID NO: 61, with from 0 to 4 amino acid modifications. In some embodiments, the heavy chain constant region comprises an amino acid sequence as set forth in SEQ ID NO: 61, with from 0 to 3 amino acid modifications. In some embodiments, the heavy chain constant region comprises an amino acid sequence as set forth in SEQ ID NO: 61, with from 0 to 2 amino acid modifications. In some embodiments, the heavy chain constant region comprises an amino acid sequence as set forth in SEQ ID NO: 61, with from 0 to 1 amino acid modifications. In some embodiments, the heavy chain constant region comprises an amino acid sequence as set forth in SEQ ID NO: 61.

In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 63. In some embodiments, the HCVR is linked to a heavy chain constant region comprising an amino acid sequence having at least 95% sequence identity to the amino acid sequence as set forth in SEQ ID NO: 63. In some embodiments, the HCVR is linked to a heavy chain constant region comprising an amino acid sequence having at least 97% sequence identity to the amino acid sequence as set forth in SEQ ID NO: 63. In some embodiments, the HCVR is linked to a heavy chain constant region comprising an amino acid sequence having at least 98% sequence identity to the amino acid sequence as set forth in SEQ ID NO: 63. In some embodiments, the HCVR is linked to a heavy chain constant region comprising an amino acid sequence having at least 99% sequence identity to the amino acid sequence as set forth in SEQ ID NO: 63. In some embodiments, the HCVR is linked to a heavy chain constant region comprising an amino acid sequence having 100% sequence identity to the amino acid sequence as set forth in SEQ ID NO: 63. In some embodiments, the HCVR is linked to a heavy chain constant region comprising an amino acid sequence as set forth in SEQ ID NO: 63, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence as set forth in SEQ ID NO: 63, with from 0 to 9 amino acid modifications. In some embodiments, the HCVR is linked to a heavy chain constant region comprising an amino acid sequence as set forth in SEQ ID NO: 63, with from 0 to 8 amino acid modifications. In some embodiments, the HCVR is linked to a heavy chain constant region comprising an amino acid sequence as set forth in SEQ ID NO: 63, with from 0 to 7 amino acid modifications. In some embodiments, the HCVR is linked to a heavy chain constant region comprising an amino acid sequence as set forth in SEQ ID NO: 63, with from 0 to 6 amino acid modifications. In some embodiments, the HCVR is linked to a heavy chain constant region comprising an amino acid sequence as set forth in SEQ ID NO: 63, with from 0 to 5 amino acid modifications. In some embodiments, the HCVR is linked to a heavy chain constant region comprising an amino acid sequence as set forth in SEQ ID NO: 63, with from 0 to 4 amino acid modifications. In some embodiments, the HCVR is linked to a heavy chain constant region comprising an amino acid sequence as set forth in SEQ ID NO: 63, with from 0 to 3 amino acid modifications. In some embodiments, the HCVR is linked to a heavy chain constant region comprising an amino acid sequence as set forth in SEQ ID NO: 63, with from 0 to 2 amino acid modifications. In some embodiments, the HCVR is linked to a heavy chain constant region comprising an amino acid sequence as set forth in SEQ ID NO: 63, with from 0 to 1 amino acid modifications. In some embodiments, the HCVR is linked to a heavy chain constant region comprising an amino acid sequence as set forth in SEQ ID NO: 63.

In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 75. In some embodiments, the HCVR is linked to a heavy chain constant region comprising an amino acid sequence having at least 95% sequence identity to the amino acid sequence as set forth in SEQ ID NO: 75. In some embodiments, the HCVR is linked to a heavy chain constant region comprising an amino acid sequence having at least 97% sequence identity to the amino acid sequence as set forth in SEQ ID NO: 75. In some embodiments, the HCVR is linked to a heavy chain constant region comprising an amino acid sequence having at least 98% sequence identity to the amino acid sequence as set forth in SEQ ID NO: 75. In some embodiments, the HCVR is linked to a heavy chain constant region comprising an amino acid sequence having at least 99% sequence identity to the amino acid sequence as set forth in SEQ ID NO: 75. In some embodiments, the HCVR is linked to a heavy chain constant region comprising an amino acid sequence having 100% sequence identity to the amino acid sequence as set forth in SEQ ID NO: 75. In some embodiments, the heavy chain constant region comprises an amino acid sequence as set forth in SEQ ID NO: 75, with from 0 to 10 amino acid modifications, such as 1, 2, 3, 4, 5, 6, 7, 8, 9 or 10 amino acid modifications. In some embodiments, the heavy chain constant region comprises an amino acid sequence as set forth in SEQ ID NO: 75, with from 0 to 9 amino acid modifications. In some embodiments, the heavy chain constant region comprises an amino acid sequence as set forth in SEQ ID NO: 75, with from 0 to 8 amino acid modifications. In some embodiments, the heavy chain constant region comprises an amino acid sequence as set forth in SEQ ID NO: 75, with from 0 to 7 amino acid modifications. In some embodiments, the heavy chain constant region comprises an amino acid sequence as set forth in SEQ ID NO: 75, with from 0 to 6 amino acid modifications. In some embodiments, the heavy chain constant region comprises an amino acid sequence as set forth in SEQ ID NO: 75, with from 0 to 5 amino acid modifications. In some embodiments, the heavy chain constant region comprises an amino acid sequence as set forth in SEQ ID NO: 75, with from 0 to 4 amino acid modifications. In some embodiments, the heavy chain constant region comprises an amino acid sequence as set forth in SEQ ID NO: 75, with from 0 to 3 amino acid modifications. In some embodiments, the heavy chain constant region comprises an amino acid sequence as set forth in SEQ ID NO: 75, with from 0 to 2 amino acid modifications. In some embodiments, the heavy chain constant region comprises an amino acid sequence as set forth in SEQ ID NO: 75, with from 0 to 1 amino acid modifications. In some embodiments, the heavy chain constant region comprises an amino acid sequence as set forth in SEQ ID NO: 75.

In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 76. In some embodiments, the HCVR is linked to a heavy chain constant region comprising an amino acid sequence having at least 95% sequence identity to the amino acid sequence as set forth in SEQ ID NO: 76. In some embodiments, the HCVR is linked to a heavy chain constant region comprising an amino acid sequence having at least 97% sequence identity to the amino acid sequence as set forth in SEQ ID NO: 76. In some embodiments, the HCVR is linked to a heavy chain constant region comprising an amino acid sequence having at least 98% sequence identity to the amino acid sequence as set forth in SEQ ID NO: 76. In some embodiments, the HCVR is linked to a heavy chain constant region comprising an amino acid sequence having at least 99% sequence identity to the amino acid sequence as set forth in SEQ ID NO: 76. In some embodiments, the HCVR is linked to a heavy chain constant region comprising an amino acid sequence having 100% sequence identity to the amino acid sequence as set forth in SEQ ID NO: 76. In some embodiments, the HCVR is linked to a heavy chain constant region comprising an amino acid sequence as set forth in SEQ ID NO: 76, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence as set forth in SEQ ID NO: 76, with from 0 to 9 amino acid modifications. In some embodiments, the HCVR is linked to a heavy chain constant region comprising an amino acid sequence as set forth in SEQ ID NO: 76, with from 0 to 8 amino acid modifications. In some embodiments, the HCVR is linked to a heavy chain constant region comprising an amino acid sequence as set forth in SEQ ID NO: 76, with from 0 to 7 amino acid modifications. In some embodiments, the HCVR is linked to a heavy chain constant region comprising an amino acid sequence as set forth in SEQ ID NO: 76, with from 0 to 6 amino acid modifications. In some embodiments, the HCVR is linked to a heavy chain constant region comprising an amino acid sequence as set forth in SEQ ID NO: 76, with from 0 to 5 amino acid modifications. In some embodiments, the HCVR is linked to a heavy chain constant region comprising an amino acid sequence as set forth in SEQ ID NO: 76, with from 0 to 4 amino acid modifications. In some embodiments, the HCVR is linked to a heavy chain constant region comprising an amino acid sequence as set forth in SEQ ID NO: 76, with from 0 to 3 amino acid modifications. In some embodiments, the HCVR is linked to a heavy chain constant region comprising an amino acid sequence as set forth in SEQ ID NO: 76, with from 0 to 2 amino acid modifications. In some embodiments, the HCVR is linked to a heavy chain constant region comprising an amino acid sequence as set forth in SEQ ID NO: 76, with from 0 to 1 amino acid modifications. In some embodiments, the HCVR is linked to a heavy chain constant region comprising an amino acid sequence as set forth in SEQ ID NO: 76.

In some embodiments, the HCVR is linked to a heavy chain constant region, wherein the heavy chain constant region comprises an Fc region that comprises one or more of the following amino acids: alanine (A) at position 234, alanine (A) at position 235, aspartic acid (D) at position 236, aspartic acid (D) at position 237, aspartic acid (D) at position 238, alanine (A) at position 265, glutamic acid (E) at position 267, glycine (G) at position 271, arginine (R) at position 330, alanine (A) at position 332, or alanine (A) at position 297 (numbering according to EU Index). In some embodiments, the Fc region comprises an aspartic acid (D) at position 238 (EU Index).

In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 77. In some embodiments, the HCVR is linked to a heavy chain constant region comprising an amino acid sequence having at least 95% sequence identity to the amino acid sequence as set forth in SEQ ID NO: 77. In some embodiments, the HCVR is linked to a heavy chain constant region comprising an amino acid sequence having at least 97% sequence identity to the amino acid sequence as set forth in SEQ ID NO: 77. In some embodiments, the HCVR is linked to a heavy chain constant region comprising an amino acid sequence having at least 98% sequence identity to the amino acid sequence as set forth in SEQ ID NO: 77. In some embodiments, the HCVR is linked to a heavy chain constant region comprising an amino acid sequence having at least 99% sequence identity to the amino acid sequence as set forth in SEQ ID NO: 77. In some embodiments, the HCVR is linked to a heavy chain constant region comprising an amino acid sequence having 100% sequence identity to the amino acid sequence as set forth in SEQ ID NO: 77. In some embodiments, the HCVR is linked to a heavy chain constant region comprising an amino acid sequence as set forth in SEQ ID NO: 77, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence as set forth in SEQ ID NO: 77, with from 0 to 9 amino acid modifications. In some embodiments, the HCVR is linked to a heavy chain constant region comprising an amino acid sequence as set forth in SEQ ID NO: 77, with from 0 to 8 amino acid modifications. In some embodiments, the HCVR is linked to a heavy chain constant region comprising an amino acid sequence as set forth in SEQ ID NO: 77, with from 0 to 7 amino acid modifications. In some embodiments, the HCVR is linked to a heavy chain constant region comprising an amino acid sequence as set forth in SEQ ID NO: 77, with from 0 to 6 amino acid modifications. In some embodiments, the HCVR is linked to a heavy chain constant region comprising an amino acid sequence as set forth in SEQ ID NO: 77, with from 0 to 5 amino acid modifications. In some embodiments, the HCVR is linked to a heavy chain constant region comprising an amino acid sequence as set forth in SEQ ID NO: 77, with from 0 to 4 amino acid modifications. In some embodiments, the HCVR is linked to a heavy chain constant region comprising an amino acid sequence as set forth in SEQ ID NO: 77, with from 0 to 3 amino acid modifications. In some embodiments, the HCVR is linked to a heavy chain constant region comprising an amino acid sequence as set forth in SEQ ID NO: 77, with from 0 to 2 amino acid modifications. In some embodiments, the HCVR is linked to a heavy chain constant region comprising an amino acid sequence as set forth in SEQ ID NO: 77, with from 0 to 1 amino acid modifications. In some embodiments, the HCVR is linked to a heavy chain constant region comprising an amino acid sequence as set forth in SEQ ID NO: 77. In some embodiments, X at position 121 of SEQ ID NO: 77 is D. In some embodiments, X at position 121 of SEQ ID NO: 77 is P. In some embodiments, X at position 329 of SEQ ID NO: 77 is G. In some embodiments, X at position 329 of SEQ ID NO: 77 is absent. In some embodiments, X at position 330 of SEQ ID NO: 77 is K. In some embodiments, X at position 330 of SEQ ID NO: 77 is absent.

In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 78. In some embodiments, the HCVR is linked to a heavy chain constant region comprising an amino acid sequence having at least 95% sequence identity to the amino acid sequence as set forth in SEQ ID NO: 78. In some embodiments, the HCVR is linked to a heavy chain constant region comprising an amino acid sequence having at least 97% sequence identity to the amino acid sequence as set forth in SEQ ID NO: 78. In some embodiments, the HCVR is linked to a heavy chain constant region comprising an amino acid sequence having at least 98% sequence identity to the amino acid sequence as set forth in SEQ ID NO: 78. In some embodiments, the HCVR is linked to a heavy chain constant region comprising an amino acid sequence having at least 99% sequence identity to the amino acid sequence as set forth in SEQ ID NO: 78. In some embodiments, the HCVR is linked to a heavy chain constant region comprising an amino acid sequence having 100% sequence identity to the amino acid sequence as set forth in SEQ ID NO: 78. In some embodiments, the HCVR is linked to a heavy chain constant region comprising an amino acid sequence as set forth in SEQ ID NO: 78, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence as set forth in SEQ ID NO: 78, with from 0 to 9 amino acid modifications. In some embodiments, the HCVR is linked to a heavy chain constant region comprising an amino acid sequence as set forth in SEQ ID NO: 78, with from 0 to 8 amino acid modifications. In some embodiments, the HCVR is linked to a heavy chain constant region comprising an amino acid sequence as set forth in SEQ ID NO: 78, with from 0 to 7 amino acid modifications. In some embodiments, the HCVR is linked to a heavy chain constant region comprising an amino acid sequence as set forth in SEQ ID NO: 78, with from 0 to 6 amino acid modifications. In some embodiments, the HCVR is linked to a heavy chain constant region comprising an amino acid sequence as set forth in SEQ ID NO: 78, with from 0 to 5 amino acid modifications. In some embodiments, the HCVR is linked to a heavy chain constant region comprising an amino acid sequence as set forth in SEQ ID NO: 78, with from 0 to 4 amino acid modifications. In some embodiments, the HCVR is linked to a heavy chain constant region comprising an amino acid sequence as set forth in SEQ ID NO: 78, with from 0 to 3 amino acid modifications. In some embodiments, the HCVR is linked to a heavy chain constant region comprising an amino acid sequence as set forth in SEQ ID NO: 78, with from 0 to 2 amino acid modifications. In some embodiments, the HCVR is linked to a heavy chain constant region comprising an amino acid sequence as set forth in SEQ ID NO: 78, with from 0 to 1 amino acid modifications. In some embodiments, the HCVR is linked to a heavy chain constant region comprising an amino acid sequence as set forth in SEQ ID NO: 78. In some embodiments, X at position 108 of SEQ ID NO: 78 is S. In some embodiments, X at position 108 of SEQ ID NO: 78 is P. In some embodiments, X at position 326 of SEQ ID NO: 78 is G. In some embodiments, X at position 326 of SEQ ID NO: 78 is absent. In some embodiments, X at position 327 of SEQ ID NO: 78 is K. In some embodiments, X at position 327 of SEQ ID NO: 78 is absent.

In some cases, the antibody or antigen-binding fragment thereof binds a region at or in proximity of C-terminus of the extracellular portion of CD200R.

In some cases, the antibody or antigen-binding fragment thereof binds a region at most 50 amino acids, 45 amino acids, 40 amino acids, 35 amino acids, 30 amino acids, 25 amino acids, 20 amino acids, or 15 amino acids from the C-terminus of the extracellular portion of CD200R.

In some cases, the antibody or antigen-binding fragment thereof binds a region about 50 amino acids, 45 amino acids, 40 amino acids, 35 amino acids, 30 amino acids, 25 amino acids, 20 amino acids, 18 amino acids, 16 amino acids, 15 amino acids, 14 amino acids, 13 amino acids, 12 amino acids, 10 amino acids, 8 amino acids, 6 amino acids, or 5 amino acids from the C-terminus of the extracellular portion of CD200R.

In some cases, the antibody or antigen-binding fragment thereof binds a region at most 100 Å, 90 Å, 80 Å, 70 Å, 60 Å, 50 Å, 40 Å, 30 Å, 20 Å, or 10 A from the cell membrane when the antibody or antigen-binding fragment thereof binds to a CD200R molecule on the cell membrane.

In some cases, the antibody or antigen-binding fragment thereof binds a region about 100 Å, 90 Å, 80 Å, 70 Å, 60 Å, 50 Å, 40 Å, 30 Å, 20 Å, or 10 A from the cell membrane when the antibody or antigen-binding fragment thereof binds to a CD200R molecule on the cell membrane.

In some cases, the antibody or antigen-binding fragment thereof binds a region in proximity of N-terminus of CD200R.

In some cases, the antibody or antigen-binding fragment thereof binds a residue of CD200R selected from T213, E230, and S194.

In some cases, the antibody or antigen-binding fragment thereof binds a residue of CD200R selected from T213 and E230.

In some cases, the antibody or antigen-binding fragment thereof does not bind to cynomolgus CD200RLa, or binds to cynomolgus CD200RLa with a K_{D} of more than 2 μM, as determined by surface plasmon resonance (SPR) at 37° C.

Disclosed herein, in some aspects, is an antibody or an antigen-binding fragment thereof of that specifically binds human CD200R or cynomolgus CD200R, wherein the antibody or antigen-binding fragment thereof does not bind to cynomolgus CD200RLa, or binds to cynomolgus CD200RLa with a K_{D} of more than 2 μM, as determined by surface plasmon resonance (SPR) at 37° C.

Disclosed herein, in some aspects, is an antibody or an antigen-binding fragment thereof of that specifically binds CD200R, wherein the antibody or antigen-binding fragment thereof binds a region at or in proximity of C-terminus of the extracellular portion of CD200R.

In some cases, the antibody or antigen-binding fragment thereof binds a residue of CD200R selected from T213 and E230.

In some cases, the antibody or antigen-binding fragment thereof binds residues, T213 and E230, of CD200R.

In some cases, the antibody or antigen-binding fragment thereof is an IgG, an IgM, an IgE, an IgA, or an IgD molecule, or is derived from one of these.

In some cases, the antibody or antigen-binding fragment thereof is an IgGI, IgG2, IgG3, or IgG4 molecule, or is derived from one of these.

In some cases, the antibody is a monoclonal antibody.

In some cases, the antibody is a human or humanized antibody.

In some cases, the antibody is a chimeric antibody.

In some cases, the antigen-binding fragment thereof is selected from the group consisting of: scFv, sc(Fv)2, dsFv, Fab, Fab′, (Fab′)2 and a diabody.

In some cases, the antibody or antigen-binding fragment thereof comprises an Fc region.

In some cases, the Fc region comprises a modification.

In some cases, the antibody or antigen-binding fragment thereof possesses increased binding to FcγR2B compared to the parent molecule that lacks the Fc region modification.

In some cases, the antibody or antigen-binding fragment thereof possesses increased ratio of binding to FcγR2B/FcγR2A, compared to the parent molecule that lacks the Fc region modification.

In some cases, the Fc region comprises one or more of the following amino acids: alanine (A) at position 234, alanine (A) at position 235, aspartic acid (D) at position 236, aspartic acid (D) at position 237 aspartic acid (D) at position 238, alanine (A) at position 265, glutamic acid (E) at position 267, glycine (G) at position 271, arginine (R) at position 330, alanine (A) at position 332, and alanine (A) at position 297, all numbering according to EU Index.

In some cases, the heavy chain or light chain further comprise a constant region.

In some cases, the heavy chain and light chain are connected by a flexible linker to form a single-chain antibody.

In some cases, the antibody or antigen-binding fragment thereof agonizes CD200R expressed on the surface of an immune cell.

In some cases, when binding to CD200R of an immune cell, the antibody or antigen-binding fragment thereof reduces activation of the immune cell relative to a comparable immune cell not bound by the antibody or antigen-binding fragment thereof.

In some cases, when binding to CD200R of an immune cell, the antibody or antigen-binding fragment thereof decreases proliferation of the immune cell relative to a comparable immune cell not bound by the antibody or antigen-binding fragment thereof.

In some cases, the reduction in activation or proliferation of the immune cell is measured by an assay described in Example 5, 16, or 17.

In some cases, the decrease in cell proliferation or activation is measured in vitro or in vivo.

In some cases, the decrease in cell proliferation or activation is at least about 10%, 15%, 20%, 25%, 30%, 40%, or 50%.

In some cases, the decrease in cell proliferation or activation is from about 10% to 50%, 10% to 40%, 10% to 30%, 10% to 20%, 10% to 15%,20% to 50%,20% to 40%, or 20% to 30%.

In some cases, when binding to CD200R of an immune cell, the antibody or antigen-binding fragment thereof reduces expression of inflammatory genes in the immune cell relative to a comparable immune cell not bound by the antibody or antigen-binding fragment thereof.

In some cases, binding of the antibody or antigen-binding fragment thereof to CD200R expressed on the surface of an immune cell decreases NFκB signaling of the immune cell relative to a comparable immune cell not bound by the antibody or antigen-binding fragment thereof.

In some cases, the decrease in NFκB signaling of the immune cell is measured by an assay described in Example 5.

In some cases, the decrease in NFκB signaling of the immune cell is at least about 10%, 15%, 20%, 25%, 30%, or 4⁰%.

In some cases, the decrease in NFκB signaling of the immune cell is from about 10% to 40%, 10% to 30%, 10% to 20%, 20% to 40%, or 30% to 40%.

In some cases, an average maximal percentage inhibition of NFκB signaling of the immune cell induced by the antibody or antigen-binding fragment thereof is at least 20%, 30%, 40%, 50%, or 60% greater than a control antibody that comprises: (a) a control heavy chain comprising CDRH1, CDRH2, and CDRH3, which comprise amino acid sequence as set forth in SEQ ID NOs: 55-57, respectively; and (b) a control light chain comprising CDRL1, CDRL2, and CDRL3, which comprise amino acid sequence as set forth in SEQ ID NOs: 58-60, respectively.

In some cases, an average maximal percentage inhibition of NFκB signaling of the immune cell induced by the antibody or antigen-binding fragment thereof is at least 20%, 30%, 40%, 50%, or 60% greater than a control antibody that comprises a control heavy chain variable region comprising amino acid sequence as set forth in SEQ ID NO: 53, and a control light chain variable region comprising amino acid sequence as set forth in SEQ ID NO: 54.

In some cases, the immune cell is a T cell or a monocyte.

In some cases, the antibody or antigen-binding fragment thereof inhibits activation of basophils.

In some cases, the antibody or antigen-binding fragment thereof inhibits activation of basophils induced by FcRI.

In some cases, the antibody or antigen-binding fragment thereof inhibits activation of basophils induced by binding of IgE to the basophils.

In some cases, the antibody or antigen-binding fragment thereof inhibits activation of basophils by at least 40% or at least 50%.

In some cases, the antibody or antigen-binding fragment thereof inhibits activation of basophils by about 10% to about 90%, about 20% to about 70%, about 30% to about 60%, or about 40% to about 60%.

In some cases, the antibody or antigen-binding fragment thereof inhibits activation of basophils by about 10%, 20%, 30%, 40%, 45%, 50%, 55% 60%, 70%, 80%, or 90%. In some cases, the inhibition of activation of basophils is measured in an assay described in Example 10.

In some cases, the antibody or antigen-binding fragment thereof does not inhibit binding of CD200 to CD200R.

In some cases, the antibody or antigen-binding fragment thereof binds human CD200R with a K_{D} of less than 10 nM, as determined by surface plasmon resonance (SPR) at 37° C.

In some cases, the antibody or antigen-binding fragment thereof binds human CD200R with a K_{D} of less than 5 nM, as determined by surface plasmon resonance (SPR) at 37° C.

In some cases, the antibody or antigen-binding fragment thereof binds human CD200R with a K_{D} of less than 2 nM, as determined by surface plasmon resonance (SPR) at 37° C.

In some cases, the antibody or antigen-binding fragment thereof binds human CD200R with a K_{D} of less than 1 nM, as determined by surface plasmon resonance (SPR) at 37° C.

In some cases, the antibody or antigen-binding fragment thereof binds human CD200R with a K_{D} of less than 0.5 nM, as determined by surface plasmon resonance (SPR) at 37° C.

In some cases, the antibody or antigen-binding fragment thereof binds cynomolgus CD200R with a K_{D} of less than 100 nM, as determined by surface plasmon resonance (SPR) at 37° C.

In some cases, the antibody or antigen-binding fragment thereof binds cynomolgus CD200R with a K_{D} of less than 1 nM, as determined by surface plasmon resonance (SPR) at 37° C.

In some cases, the antibody or antigen-binding fragment thereof binds cynomolgus CD200R with a K_{D} of less than 0.1 nM, as determined by surface plasmon resonance (SPR) at 37° C.

In some cases, the antibody or antigen-binding fragment thereof binds cynomolgus CD200R with a K_{D} of less than 0.01 nM, as determined by surface plasmon resonance (SPR) at 37° C.

In some cases, the antibody or antigen-binding fragment thereof does not induce significant cytokine release when the antibody or antigen-binding fragment thereof binds to CD200R on the surface of an immune cell.

In some cases, the antibody or antigen-binding fragment thereof comprises a domain that binds to an Fc receptor.

In some cases, the Fc receptor is expressed on the surface of an immune cell.

In some cases, the immune cell is an antigen presenting cell.

In some cases, the antigen presenting cell is a dendritic cell, macrophage, monocyte, or neutrophil.

In some cases, the binding of the antibody or antigen-binding fragment thereof to the Fc receptor expressed on the surface of the immune cell and binding to CD200R on the surface of a second immune cell results in the cell surface of the immune cell and the cell surface of the second immune cell to be within 250 Å, 200 Å, 150 Å, or 100 Å.

In some cases, the binding of the antibody or antigen-binding fragment thereof to the Fc receptor expressed on the surface of the immune cell and binding to CD200R on the surface of a second immune cell results in the cell surface of the immune cell and the cell surface of the second immune cell to be within 250 Å, 200 Å, 150 Å, or 100 Å.

In some cases, the Fc receptor is FcγRIIB.

In some cases, the antibody or antigen-binding fragment thereof is bi-specific or multi-specific.

Disclosed herein, in some aspects, is an isolated nucleic acid that comprises one or more nucleotide sequences encoding polypeptides capable of forming the antibody or antigen-binding fragment thereof disclosed herein.

Disclosed herein, in some aspects, is a vector that comprises one or more nucleotide sequences encoding polypeptides capable of forming the antibody or antigen-binding fragment thereof disclosed herein.

Disclosed herein, in some aspects, is a host cell comprising one or more nucleic acid molecules encoding the amino acid sequence of a heavy chain and a light chain which when expressed are capable of forming the antibody or antigen-binding fragment thereof disclosed herein.

Disclosed herein, in some aspects, is a method, comprising culturing the host cell disclosed herein under conditions for production of the antibody or antigen-binding fragment thereof.

Disclosed herein, in some aspects, is a method, comprising:
(a) providing a host cell comprising one or more nucleic acid molecules encoding the amino acid sequence of a heavy chain and a light chain which when expressed are capable of forming the antibody or antigen-binding fragment thereof disclosed herein;
(b) culturing the host cell expressing the encoded amino acid sequence; and
(c) isolating the antibody or the antigen-binding fragment thereof.

Disclosed herein, in some aspects, is an immunoconjugate comprising the antibody or antigen-binding fragment thereof disclosed herein conjugated with an agent.

Disclosed herein, in some aspects, is a pharmaceutical composition comprising a therapeutically effective amount of the antibody or antigen-binding fragment thereof disclosed herein or the immunoconjugate disclosed herein, and at least one pharmaceutically acceptable excipient.

Disclosed herein, in some aspects, is an antibody or antigen-binding fragment thereof disclosed herein or the immunoconjugate disclosed herein or a pharmaceutical composition disclosed herein for use in therapy.

Disclosed herein, in some aspects, is a kit comprising the antibody or antigen-binding fragment thereof disclosed herein, the immunoconjugate disclosed herein, or the pharmaceutical composition disclosed herein in a container.

In some cases, the kit further comprises an informational material containing instructions for use of the antibody or antigen-binding fragment thereof disclosed herein, the immunoconjugate disclosed herein, or the pharmaceutical composition disclosed herein.

Disclosed herein, in some aspects, is a method of treating a disease or condition in a subject in need thereof, comprising administering to the subject a therapeutically effective amount of the antibody or antigen-binding fragment thereof disclosed herein or the immunoconjugate disclosed herein, or administering to the subject the pharmaceutical composition disclosed herein.

In some cases, the disease or condition comprises a disease or condition associated with CD200R.

In some cases, the disease or condition comprises an autoimmune disease or condition or an inflammatory disease or condition.

In some cases, the disease or condition comprises acute disseminated encephalomyelitis (ADEM), Addison's disease, allergy, alopecia areata, amyotrophic lateral sclerosis, ankylosing spondylitis, anti-phospholipid syndrome, asthma, autoimmune haemolytic anaemia, autoimmune hepatitis, autoimmune pancreatitis, autoimmune polyendocrine syndrome, Behcet's disease, bullous pemphigoid, cerebral malaria, chronic inflammatory demyelinating polyneuropathy, coeliac disease, Crohn's disease, Cushing's Syndrome, dermatitis herpetiformis, dermatomyositis, diabetes mellitus type 1, eosinophilic granulomatosis with polyangiitis, gallbladder disease, graft versus host disease, Graves' disease, Guillain-Barre syndrome, Hashimoto's thyroiditis, Hidradenitis Suppurativa, inflammatory bowel disease (IBD), inflammatory fibrosis, irritable bowel syndrome, juvenile arthritis, Kawasaki disease, leukemia, lyme arthritis, lymphoma, lymphoproliferative disorders, meningoencephalitis, multiple sclerosis, myasthenia gravis, myeloma, neuromyelitis optica, pelvic inflammatory disease, pemphigus, peritonitis, Pilonidal disease, polymyositis, primary biliary cholangitis, primary sclerosing cholangitis, psoriasis, psoriatic arthritis, rheumatoid arthritis, sarcoidosis, Sjogren's syndrome, systemic lupus erythematosus, Takayasu's arteritis, temporal arteritis, transplant rejection, transverse myelitis, ulcerative colitis, uveitis, vasculitis, vitiligo and Vogt-Koyanagi-Harada Disease.

In some cases, the disease or condition comprises an autoimmune skin disease or condition.

In some cases, the autoimmune skin disease or condition comprises Behcet's disease, dermatitis herpetiformis, dermatomyositis, epidermolysis bullosa, lichen planus, linear IgA disease, lupus of the skin, morphea/scleroderma, ocular cicatrical pemphigoid, pemphigoid, bullous pemphigoid, pemphigus, psoriasis, scleroderma, or vasculitis.

In some cases, the subject is a human subject.

Disclosed herein, in some aspects, is a method of downregulating an immune response in a subject, comprising administering the subject the antibody or antigen-binding fragment thereof or the immunoconjugate disclosed herein, or administering to the subject the pharmaceutical composition disclosed herein.

Disclosed herein, in some aspects, is a method of suppressing an immune cell that expresses CD200R, comprising contacting the immune cell with the antibody or antigen-binding fragment thereof disclosed herein or the immunoconjugate disclosed herein.

In some cases, the immune cell comprises a T cell, a B cell, or a macrophage.

In some cases, the immune cell comprises an antigen-specific T cell.

In some cases, the subject is a human subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1B show surface plasmon resonance (SPR) curves that demonstrate binding of CD200 by CD200R that is injected over and captured by immobilized anti-CD200R antibody for a non-competing clone (FIG. 1A) and a competing clone (FIG. 1B), respectively. For a non-competing clone (exemplified in FIG. 1A), CD200R captured by the non-competing CD200R antibodies can bind to its natural ligand CD200, thus showing an increase in SPR signal on the graph, while for a competing clone (exemplified in FIG. 1B), CD200R captured by the competing CD200R antibodies cannot bind to its natural ligand CD200, thus no significant change in SPR signal on the graph.

FIG. 2 shows the epitope map of clone 21.3.1 as defined by surface mutagenesis. The mutated surface residues that reduce binding of the antibody by >90% relative to wild type receptor are indicated by residue number (E230 and T213), while mutated surface residues that have no effect on antibody binding are shaded but not indicated by residue number. The epitope was found to be near the C-terminus of CD200R very close to the likely position of the cell membrane. As shown, the orientation of the receptor with respect to the cell membrane is predicted by positioning the C-terminus of the extracellular portion in proximity to the membrane and the N-terminus distal to the membrane.

FIG. 3 shows alignment of exemplary humanized VH and VL variants to the murine parental clone 21.3.1 variable domain sequences.

FIGS. 4A-4D show predicted immunogenicity, based on HLA-DR binding scores, for the murine parental variable domains and exemplary humanized variable domains of clone 21.3.1.

FIGS. 5A-5D demonstrate the inhibition of FcεRI-induced basophil activation. FIG. 5A is a schematic overview of the experiment. FIG. 5B show images of basophils from an individual donor 30 minutes after IgE stimulation. FIGS. 5C-5D show plots quantifying basophil activation, expressed as the count of CD63+cells (light grey objects in FIG. 5B) divided by the total cell count (phase objects in FIG. 5B). Results for individual donors are plotted in FIG. 5C. Normalized AUCs were summarized in FIG. 5D.

FIGS. 6A-6C illustrate gene pathways regulated by CD200R signaling. FIG. 6A shows results from RNA sequencing that confirmed the agonistic effect of an exemplary CD200R agonistic antibody according to some embodiments of the present disclosure, with the identification of 2080 differentially expressed genes (899 upregulated, 1181 downregulated) when comparing stimulated THP-1 cells in the presence of CD200R agonistic antibody vs isotype control. FIG. 6B shows gene set enrichment analysis, which revealed that CD200R agonism by the CD200R agonistic antibody downregulated key inflammatory pathways associated with chronic inflammatory conditions. FIG. 6C shows that CD200R1 is upregulated in skin biopsies from atopic dermatitis patients.

FIG. 7 shows binding curves for CD200R antibodies, or relevant isotype controls, to human CD200R expressing Jurkat T cells, as assessed by flow cytometry.

FIG. 8 shows the output of an enzyme fragment complementation assay which reports DOK2 recruitment to the cytoplasmic tail of CD200R in a Jurkat T cell line.

### DETAILED DESCRIPTION

Disclosed herein, in some aspects, are methods, compositions, kits, vectors, polynucleotides, and host cells relating to an antibody recognizing immune cell receptor CD200R. In some embodiments, the antibody or antigen-binding fragment thereof is an agonist antibody. In some embodiments, the agonist antibody specifically binds to CD200R. In some embodiments, the agonist antibody specifically binds to human CD200R.

In some embodiments, the agonist anti-CD200R antibody disclosed herein enhances or activates the CD200R signaling pathway. In some embodiments, the anti-CD200R antibody downregulates immune response. In some embodiments, the anti-CD200R antibody downregulates inflammation. In some embodiments, the anti-CD200R antibody downregulates NF-KB signaling. In some embodiments, the anti-CD200R antibody decreases immune cell proliferation relative to a comparable immune cell not bound by the anti-CD200R antibody.

Without wishing to be bound by a certain theory, the anti-CD200R antibodies disclosed herein enhance CD200R signaling pathway by enhancing activation of CD200R or signaling pathway induced by activated CD200R, e.g., interaction of Dok2 with the membrane distal tyrosine residue located within a phosphotyrosine-binding (PTB) domain recognition motif (NPxY), phosphorylation of tyrosine residues of CD200R, and/or recruitment of SHIP and RasGAP. In some cases, the anti-CD200R antibodies bind CD200R on the surface of an immune cell. In some cases, the anti-CD200R antibodies bind another surface protein expressed on the surface of another immune cell. In some cases, anti-CD200R antibodies can bring the surfaces of the two immune cells into close proximity. In some embodiments, such close proximity is 250 Å, 200 Å, 150 Å, or 100 Å. In some cases, close proximity of the two immune cells reduces bulky phosphatase(s) at or near the immune synapse between the two immune cells. In some embodiments, bulky phosphatase contributes to dephosphorylation of the CD200R. In some embodiments, decreased bulky phosphatase leads to more phosphorylation of CD200R, which in turn enhances the CD200R signaling pathway. In some embodiments, the immune cell is a T cell. In some cases, the immune cell is a monocyte.

In some embodiments, the anti-CD200R antibody recognizes an extracellular region of human CD200R. In some embodiments, the antibody recognizes an extracellular region of cynomolgus CD200R. In some embodiments, the anti-CD200R antibody does not compete with CD200R's natural ligand. In some cases, the antibody binds a region near the C-terminus of CD200R, or C-terminus of the extracellular portion of CD200R. In some cases, the anti-CD200R antibody binds a region close to the likely position of the cell membrane. In some cases, antibody binds a region close to the N-terminus of domain 2. When expressed on the surface of a cell membrane, CD200R can have extracellular portion, transmembrane portion, and intracellular portion. CD200R can have two IgSF domains in its extracellular portion when expressed on the surface of a cell membrane, domain 1 and domain 2, e.g., as described in Wright et al. J Immunol 2003; 171:3034-3046, which is incorporated herein by reference in its entirety. Domain 1 is the N-terminal domain in the extracellular portion of CD200R and domain 2 is the C-terminal domain in the extracellular portion of CD200R. CD200R domain 2 is the more membrane proximal domain.

In other cases, the anti-CD200R antibody binds a region more distal from the cell membrane. In some embodiments, the anti-CD200R binds a T213 residue of CD200R. In some embodiments, the anti-CD200R binds a E230 residue of CD200R. In some embodiments, the anti-CD200R binds a T213 residue and a E230 residue of CD200R. In some embodiments, the anti-CD200R binds a S194 residue of CD200R.

In some embodiments, the CD200R agonist antibodies disclosed are more efficacious than currently existing antibodies (e.g., I-4P described in Example 5) at promoting the CD200R inhibitory signaling toward immune cells and/or the immune system, downregulating immune cell responses. In some cases, the anti-CD200R antibodies disclosed herein enhance CD200R's inhibition of the NF-κB pathway more effectively than currently existing antibodies (e.g., I-4P). The anti-CD200R antibodies disclosed herein can be particularly useful in the treatment of immune mediated, and/or CD200R associated disorders, or diseases generated by aberrant immune pathologies or having cancerous origins.

In some embodiments, the antibody is a human antibody. In some embodiments, the CD200R antibody disclosed herein is a humanized antibody. In some embodiments, the CD200R agonist antibody is a monoclonal antibody. In some embodiments, the CD200R antibody is a chimeric antibody. In some embodiments, the CD200R antibody is generated in mice. In some embodiments, the CD200R antibody comprises one or more modifications in its Fc region. In some embodiments, the CD200R antibody comprises one or more modifications in its heavy chain. In some embodiments, the CD200R antibody comprises one or more modifications in its light chain.

In some embodiments, the CD200R antibody is an IgG molecule or is derived from an IgG molecule. In some embodiments, the CD200R antibody is an IgM molecule or is derived from an IgM molecule. In some embodiments, the CD200R antibody is an IgE molecule or is derived from an IgE molecule. In some embodiments, the CD200R antibody is an IgA molecule or is derived from an IgA molecule. In some embodiments, the CD200R antibody is an IgD molecule or is derived from an IgD molecule.

In some aspects, disclosed herein are compositions, systems, pharmaceutical compositions, method of treatments, kits, and methods of manufacturing that relate to CD200R antibodies.

It is to be understood that one, some, or all of the properties of the various embodiments described herein may be applied to any aspect unless the content clearly dictates otherwise. Furthermore, that the various embodiments may be combined to form other embodiments of the present disclosure. These and other aspects of the disclosure will become apparent to one of skill in the art. These and other embodiments of the disclosure are further described by the detailed description that follows.

### Definitions

The terms “agonist,” “agonistic,” “agonize,” and other grammatically equivalents, as used herein, refer to or relate to an agent that can bind to a receptor or any other protein target, and activate or enhance an activity of, or help initiate activation of, the receptor or protein target. In some cases, an agonist can promote the receptor or other protein target that it binds to, to induce a biological response, e.g., signal transduction or other changes in cellular activities. As used herein, a CD200R agonist antibody (or antibody fragment) refers to an antibody (or antibody fragment) that binds to CD200R expressed on the surface of an immune cell and enhances its inhibitory signal to the immune cell, including without limitation T cells, macrophages and/or B lymphocytes.

In the present disclosure, wherever aspects are described herein with the language “comprising,” otherwise analogous aspects described in terms of “consisting of” and/or “consisting essentially of” are also provided. All definitions herein described whether specifically mentioned or not, should be construed to refer to definitions as used throughout the specification and attached claims.

Throughout the specification and attached claims, the singular form “a”, “an” and “the” include plural references unless the context clearly dictates otherwise. For example, the term “a cell” includes a plurality of cells, including mixtures thereof.

In the present disclosure, one, some, or all of the properties of the various embodiments described herein may be applied to any aspect unless the content clearly dictates otherwise. Furthermore, that the various embodiments may be combined to form other embodiments of the present disclosure. These and other aspects of the disclosure will become apparent to one of skill in the art. These and other embodiments of the disclosure are further described by the detailed description herein.

Throughout the specification and attached claims, and unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of ordinary skill with a general dictionary of many of the terms used in this disclosure.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The numbering of amino acids in the variable domain, CDRs and framework regions (FRs), of an antibody follow, unless otherwise indicated, the Kabat definition as set forth in Kabat et al. Sequences of Proteins of Immunological Interest, 5th Ed. Public Health Service, National Institutes of Health, Bethesda, MD. (1991).

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides as described herein are based upon an antibody, the polypeptides can occur as single chains or associated chains.

The term “amino acid” refers to natural, unnatural, and synthetic amino acids, including but not limited to both the D or L optical isomers, and amino acid analogs and peptidomimetics. Standard single or three letter codes are used to designate amino acids.

A “variant” when applied to a protein is a protein with sequence homology to the native biologically active protein that retains at least a portion of the therapeutic and/or biological activity of the biologically active protein. For example, a variant protein may share at least 70%, 75%, 80%, 85%, 90%, 95%, 96%, 97%, 98% or 99% amino acid sequence identity compared with the reference biologically active protein or any ranges in between the at least 70% and 99%. As used herein, the term “biologically active protein moiety” includes proteins modified deliberately, as for example, by site directed mutagenesis, synthesis of the encoding gene, insertions, or accidentally through mutations.

In the context of polypeptides, a “linear sequence” or a “sequence” is an order of amino acids in a polypeptide in an amino to carboxyl terminus direction in which residues that neighbor each other in the sequence are contiguous in the primary structure of the polypeptide. A “partial sequence” is a linear sequence of part of a polypeptide that is known to comprise additional residues in one or both directions.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda MD)); and (2) an approach based on crystallographic studies of antigen-antibody complexes (Al-lazikani et al (1997) J. Molec. Biol. 273:927-948)). As used herein, a CDR may refer to CDRs defined by either approach or by a combination of both approaches.

A “constant region” of an antibody refers to the constant region of the antibody light chain or the constant region of the antibody heavy chain, either alone or in combination.

A “host cell” includes an individual cell or cell culture that can be or has been a recipient for vector(s) comprising exogenous polynucleotides. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of the present disclosure.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. (Kabat et al., Sequences of Proteins of Immunological Interest, 5th Ed. Public Health Service, National Institutes of Health, Bethesda, Md., 1991.) The Fc region of an immunoglobulin generally comprises two constant domains, CH₂ and CH₃.

A “functional Fc region” possesses at least one effector function of a native sequence Fc region. Exemplary “effector functions” include C1q binding; complement dependent cytotoxicity (CDC); Fc receptor binding; antibody-dependent cell-mediated cytotoxicity (ADCC); phagocytosis; down-regulation of cell surface receptors (e.g., B cell receptor; BCR), etc. Such effector functions generally require the Fc region to be combined with a binding domain (e.g., an antibody variable domain) and can be assessed using various assays known in the art for evaluating such antibody effector functions.

A “native sequence Fc region” comprises an amino acid sequence identical to the amino acid sequence of an Fc region found in nature. A “variant Fc region” comprises an amino acid sequence which differs from that of a native sequence Fc region by virtue of at least one amino acid modification yet retains at least one effector function of the native sequence Fc region. Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95%, at least about 96%, at least about 97%, at least about 98%, at least about 99% sequence identity and sequence identity between said ranges therewith.

An “individual” or a “subject” is a mammal, more preferably a human. Mammals also include, but are not limited to, farm animals, sport animals, pets, primates, horses, dogs, cats, mice and rats.

As used herein, “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term “effective amount” also applies to a dose that will provide an image for detection by an appropriate imaging method. The specific dose may vary depending on one or more of: the particular agent chosen, the dosing regimen to be followed, whether it is administered in combination with other compounds, timing of administration, the tissue to be imaged, and the physical delivery system in which it is carried. An effective amount of an active agent may be administered in a single dose or in multiple doses. A therapeutically effective amount of antibody ranges from about 0.001 to about 25 mg/kg body weight, preferably from about 0.01 to about 25 mg/kg body weight, from about 0.1 to about 20 mg/kg body weight, or from about 1 to about 10 mg/kg. The dosage may be adjusted, as necessary, to suit observed effects of the treatment and/or as most effective to provide a cure, prevention, control symptoms and the like as determined by one of ordinary skills in the art. The appropriate dose is chosen based on clinical indications by a treating physician or person of skill in the art. A component may be described herein as having at least an effective amount, or at least an amount effective to produce a desired result, such as that associated with a particular goal or purpose, such as any described herein. The desired therapeutic result herein can include, without limitation, to treating, alleviating, or curing a disorder, cancer, an immune-associated disease, a CD200R associated disorder, and/or any symptoms from immune-related pathologies and the like as described in this specification and or appended claims.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, P A, 1990; and Remington, The Science and Practice of Pharmacy 20th Ed. Mack Publishing, 2000).

Throughout the specification and attached claims, the methods and systems of this disclosure as described herein may employ, unless otherwise indicated, conventional techniques and descriptions of molecular biology (including recombinant techniques), cell biology, biochemistry, microarray and sequencing technology, which are within the skill of those who practice in the art. Such conventional techniques include polymer array synthesis, hybridization and ligation of oligonucleotides, sequencing of oligonucleotides, and detection of hybridization using a label. Specific illustrations of suitable techniques can be had by reference to the examples herein. However, equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, et al., Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Green, Molecular Cloning: A Laboratory Manual, 4th Edition (2012) (all from Cold Spring Harbor Laboratory Press); Stryer, L., Biochemistry (4th Ed.) W. H. Freeman, N.Y. (1995); Gait, “Oligonucleotide Synthesis: A Practical Approach” IRL Press, London (1984); Nelson and Cox, Lehninger, Principles of Biochemistry, 6^{th} Ed., W. H. Freeman Pub., New York (2012); R. I. Freshney, Culture of Animal Cells: A Manual of Basic Technique and Specialized Applications, 6^{th} Ed., Wiley-Blackwell (2010); and Berg et al., Biochemistry, 5^{th} Ed., W. H. Freeman Pub., New York (2002), all of which are herein incorporated by reference in their entirety for all purposes. Before the present compositions, research tools and systems and methods are described, it is to be understood that this disclosure is not limited to the specific systems and methods, compositions, targets and uses described, as such may, of course, vary. It is also to be understood that the terminology used herein is for the purpose of describing particular aspects only and is not intended to limit the scope of the present disclosure, which will be limited only by appended claims.

The term “anti-CD200R antibody” or molecule as used herein, refers to both antibodies and binding fragments thereof capable of binding to CD200R.

In the present disclosure, an “antibody” refers to an immunoglobulin molecule capable of specific binding to a target, such as a carbohydrate, polynucleotide, lipid, polypeptide, etc., through at least one antigen recognition site, located in the variable region of the immunoglobulin molecule. The term as used herein, includes an immunoglobulin molecule that specifically binds to an antigen and comprises an FcR binding site which may or may not be functional. As used in the disclosure, the term encompasses not only intact polyclonal or monoclonal antibodies, but also fragments thereof (such as Fab, Fab′, F(ab′)2, diabodies) Fv fragments and single chain (ScFv) mutants that contain an antigen recognition site or antigen binding site and have ability to bind to an antigen. Antigen-binding antibody or immunoglobulin fragments are well known in the art; such fragment can have a functional or non-functional Fc receptor binding site. Further as used herein, the term is not limited only to intact polyclonal or monoclonal antibodies, multispecific antibodies such as bispecific, or polyspecific antibodies generated from at least two intact antibodies, chimeric antibodies, humanized antibodies, single-chain, chimeric, synthetic, recombinant, hybrid, mutated, grafted antibodies, human antibodies, and any other modified immunoglobulin molecule comprising an antigen binding site so long as the antibodies exhibit the desired biological activity.

There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Unless dictated otherwise by contextual constraints the antibodies of the disclosure can be from one of these classes or subclasses of antibodies. Heavy-chain constant domains that correspond to the different classes of antibodies are typically denoted by the corresponding lower-case Greek letter α, δ, ε, γ, and μ, respectively. Light chains of the antibodies from any vertebrate species can be assigned to one of two clearly distinct types, called kappa (κ) and lambda (λ), based on the amino acid sequences of their constant domains.

Throughout the specification and appended claims, “Fc receptor” and “FcR” describe a receptor that binds to the Fc region of an antibody. FcRs are reviewed in Ravetch and Kinet, 1991, Ann. Rev. Immunol., 9:457-92; Capel et al., 1994, Immunomethods, 4:25-34; and de Haas et al., 1995, J. Lab. Clin. Med., 126:330-41. “FcR” also includes the neonatal receptor, FcRn, which is responsible for the transfer of maternal IgGs to the fetus (Guyer et al., 1976, J. Immunol., 117:587; and Kim et al., 1994, J. Immunol., 24:249).

Wherever used herein, “monoclonal antibody” refers to an antibody obtained from a population of substantially homogeneous antibodies. In general, the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies and is not to be construed as requiring production of the antibody by any particular method. For example, the monoclonal antibodies to be used in accordance with the present disclosure may be made by the hybridoma method first described by Kohler and Milstein, 1975, Nature, 256:495, or may be made by recombinant DNA methods such as described in U.S. Pat. No. 4,816,567. The monoclonal antibodies may also be isolated from phage libraries generated using the techniques described in McCafferty et al., 1990, Nature, 348:552-554, for example.

Wherever used herein, “human antibody” means an antibody having an amino acid sequence corresponding to that of an antibody produced by a human and/or has been made using any of the techniques for making human antibodies known in the art or of the present disclosure. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In some cases, the human antibody is selected from a phage library, where that phage library expresses human antibodies (Vaughan et al., 1996, Nature Biotechnology, 14:309-314; Sheets et al., 1998, PNAS, (USA) 95:6157-6162; Hoogenboom and Winter, 1991, J. Mol. Biol., 227:381; Marks et al., 1991, J. Mol. Biol., 222:581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., 1991, J. Immunol., 147 (1):86-95; and U.S. Pat. No. 5,750,373.

As used herein, “humanized” antibodies refer to forms of non-human (e.g., murine) antibodies that are specific chimeric immunoglobulins, immunoglobulin chains, or fragments thereof (such as Fv, Fab, Fab′, F(ab′)2 or other antigen-binding subsequences of antibodies) that contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and biological activity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs “derived from” one or more CDRs from the original antibody.

As used herein, the term “chimeric antibody” can refer to antibodies in which the variable region sequences are derived from one species and the constant region sequences are derived from another species, such as an antibody in which the variable region sequences are derived from a mouse antibody and the constant region sequences are derived from a human antibody. In some embodiments, the antibody provided herein is a monoclonal antibody.

Wherever used herein “antibody-dependent cell-mediated cytotoxicity” and “ADCC” refer to a cell-mediated reaction in which nonspecific cytotoxic cells that express Fc receptors (FcRs) (e.g., natural killer (NK) cells, neutrophils, and macrophages) recognize bound antibody on a target cell and subsequently cause lysis of the target cell. ADCC activity of a molecule of interest can be assessed using an in vitro ADCC assay, such as that described in U.S. Pat. No. 5,500,362 or 5,821,337. Useful effector cells for such assays include peripheral blood mononuclear cells (PBMC) and NK cells. Alternatively, or additionally, ADCC activity of the molecule of interest may be assessed in vivo, e.g., in an animal model such as that disclosed in Clynes et al., 1998, PNAS (USA), 95:652-656.

“Complement dependent cytotoxicity” and “CDC” refer to the lysing of a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santoro et al., J. Immunol. Methods, 202:163 (1996), may be performed.

An antibody that “specifically binds” to an epitope is a term well understood in the art, and methods to determine such specific binding are also well known in the art. A molecule is said to exhibit “specific binding” if it reacts or associates more frequently, more rapidly, with greater duration and/or with greater affinity with a particular cell, protein or substance than it does with alternative cells, proteins or substances. An antibody “specifically binds” or “preferentially binds” to a target if it binds with greater affinity, avidity, more readily, and/or with greater duration than it binds to other substances. For example, an antibody that specifically or preferentially binds to CD200R is an antibody that binds this epitope with greater affinity, avidity, more readily, and/or with greater duration than it binds to other epitopes. As a further example, an antibody (or other moiety) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

A “fragment” when applied to a protein, is a truncated form of a native biologically active protein that may or may not retain at least a portion of the therapeutic and/or biological activity. Herein, the terms “antibody fragment molecules of the disclosure”, “antibody fragment” and “antigen-binding fragment thereof”, are used interchangeably.

Sequence Identity

The sequence identity with respect to the anti-CD200R antibody or any other amino acid sequences identified herein, is defined as the percentage of amino acid residues in a query sequence that are identical with the amino acid residues of a second, reference polypeptide sequence or a portion thereof, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Percent identity may be measured over the length of an entire defined polypeptide sequence, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured. In some embodiments, percent identity is determined with respect to the full length of a noted reference sequence, such as a sequence provided herein. For example, sequence comparison between two amino acid sequences (or a shorter length thereof) of the present disclosure may be carried out by computer program Blastp (protein-protein BLAST) provided online by Nation Center for Biotechnology Information (NCBI). The percentage amino acid sequence identity of a given amino acid sequence A to a given amino acid sequence B (which can alternatively be phrased as a given amino acid sequence A that has a certain % amino acid sequence identity to a given amino acid sequence B) is calculated by the formula as follows:

$$\frac{X}{Y} \times 100 \%$$

where X is the number of amino acid residues scored as identical matches by the sequence alignment program BLAST in that program's alignment of A and B, and where Y is the total number of amino acid residues in A or B, whichever is shorter.
Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity.

CD200 and CD200R

In some aspects, provided herein are compositions and methods related to antibodies or antibody fragments that bind to and agonize CD200R, a receptor that can be present on the surface of immune cells including T cells, natural killer cells, B cells, monocytes, myeloid cells, macrophages, microglia and dendritic cells. CD200:CD200R plays a critical role in limiting immune cell proliferation, immune response, and inflammation.

Without wishing to be bound by a certain theory, activation of the CD200R signaling pathway leads to inhibition of immune cell activation and proliferation. Antibodies that enhance CD200R signaling has the potential to downregulate inflammation and immune response in infection, arthritis, cancer, autoimmune diseases, transplant rejection, or other disease or condition associated with CD200R.CD200 is a member of the immunoglobulin superfamily (IgSF) and a transmembrane type 1a glycoprotein and can be highly expressed in a variety of cells types, for example, dendritic cells, macrophages, B lymphocytes, neuronal and endothelial cells, as well as in some T lymphocytes. CD200 has been implicated in inhibitory signaling which prohibits or reduces expression of genes involved in inflammatory responses. CD200 can function in concert with its natural receptor CD200R to activate the immunosuppression signaling cascade.

Without wishing to be bound by a certain theory, in some cases, CD200R does not contain any immunoreceptor tyrosine-based inhibitory motifs (ITIMs), which are usually present in a large number of inhibitory receptors, and which can mediate their inhibitory roles through the recruitment of protein tyrosine phosphatases such as Src homology 2 domain-containing phosphatase (SUP) 1, SH2, or the inositol phosphatase (SHIP) upon phosphorylation. Instead, in some cases, the molecular signaling mechanism of CD200R following activation involves direct interaction of the adaptor protein downstream to tyrosine kinase (Dok2), with the CD200R membrane distal tyrosine residue located within a phosphotyrosine-binding (PTB) domain recognition motif (NPxY). This interaction can lead to binding and recruitment of RAS p21 protein activator (RasGAP) which is an SH2 domain containing protein. The formation of the Dok2-RasGAP complex can inhibit Ras activation, leading to inhibition of other downstream inflammatory signals through inhibition of principal mitogen activated protein kinases including Phosphoinositide 3-kinase (PI3K) and Extracellular Signal-regulated Kinase (Erk). In some cases, the interaction between CD200 and CD200R induces phosphorylation of tyrosine residues, initiating a signaling cascade which recruits SHIP and RasGAP. Dok2 can be regulated by Dok1 through Crk Like (CrkL)-RasGAP suppression; both Dok2 and Dok1 can be recruited during CD200-CD200R interaction that can lead to recruitment of RasGAP and SH2-containing inositol phosphatase. In some cases, Dok1 activation is initiated through binding to one of the three phosphotyrosine residues located on the cytoplasmic amino acid chain of CD200R. This Dokl-phosphotyrosine binding can then suppress Dok2's effect on Ras through activation of CrkL.

In some aspects, provided herein are antibodies, compositions, nucleic acids, vectors, host cells, kits, uses thereof, and methods of making the same that can circumvent some of the aforementioned and other problems known in the art that are associated with existing anti-CD200R antibodies. In some embodiments, provided herein are CD200R agonist antibodies that enhance CD200R signaling by binding to CD200R on immune cells without competing with CD200.

Antibody Sequence

In the present disclosure, is provided herein compositions, therapeutics, kits, vectors, nucleic acid sequences, manufacturing, culturing and/or methods for producing an CD200R agonist antibody or an antigen-binding fragment or a functional fragment thereof that enhances the biological effects of CD200R activation, e.g., inhibiting the activity or proliferation of the immune cell that expresses the CD200R molecule the antibody binds to, or promoting downregulation of CD200R expressing immune cell responses and inflammatory responses. In some cases, a CD200R agonist antibody promotes the downstream signaling of CD200R that is triggered by CD200 binding. In some cases, a CD200R agonist antibody enhances the interaction between CD200 and CD200R. In some cases, a CD200R agonist antibody promotes the downstream signaling of CD200R without increasing or enhancing the interaction between CD200 and CD200R. In some cases, a CD200R agonist antibody activates or enhances CD200R signaling in the absence of CD200 binding to CD200R.

In some embodiments, an antibody or an antigen-binding fragment (e.g., an isolated antibody) provided herein specifically binds to CD200R and enhances the CD200R signaling pathway of the cell has CD200R expressed on its surface.

In some embodiments, an antibody or an antigen-binding fragment (e.g., an isolated antibody) provided herein is a CD200R antibody that comprises a heavy chain. In some embodiments, an antibody or an antigen-binding fragment provided herein is a CD200R antibody that comprises a light chain. In some embodiments, an antibody or an antigen-binding fragment provided herein is a CD200R antibody that comprises an Fc region.

In some embodiments, the heavy chain further comprises at least one heavy chain complementarity determining region (CDR). In some embodiments, the heavy chain CDR comprises a sequence as set forth in any of SEQ ID NO: 3, 4, 41, 5, 11, 12, 13, 19, 20, 21, 27, 28, 29, 35, 36, 36, 69, 70, or 92, with from 0 to 3 amino acid modifications. In some embodiments, the heavy chain CDR comprises a sequence as set forth in any of SEQ ID NO: 3, 4, 41, 5, 11, 12, 13, 19, 20, 21, 27, 28, 29, 35, 36, 36, 69, 70, or 92, with from 0 to 2 amino acid modifications. In some embodiments, the heavy chain CDR comprises a sequence as set forth in any of SEQ ID NO: 3, 4, 41, 5, 11, 12, 13, 19, 20, 21, 27, 28, 29, 35, 36, 36, 69, 70, or 92, with from 0 to 1 amino acid modifications.

In some embodiments, the heavy chain comprises a heavy chain variable region. In some cases, the heavy chain variable region comprises heavy chain complementarity determining region 1 (CDRH1), CDRH2, and CDRH3. In some cases, CDRH1, CDRH2, and CDRH3 comprise the sequence as set forth in SEQ ID NOs: 3, 41, and 70, respectively, each with 0 to 3 amino acid modifications, such as 0, 1, 2, or 3 modifications. In some cases, CDRH1, CDRH2, and CDRH3 comprise the sequence as set forth in SEQ ID NOs: 3, 41, and 5, respectively, each with 0 to 3 amino acid modifications, such as 0, 1, 2, or 3 modifications. In some cases, CDRH1, CDRH2, and CDRH3 comprise the sequence as set forth in SEQ ID NOs: 3, 4, and 5, respectively, each with 0 to 3 amino acid modifications, such as 0, 1, 2, or 3 modifications. In some cases, CDRH1, CDRH2, and CDRH3 comprise the sequence as set forth in SEQ ID NOs: 3, 41, and 5, respectively, each with 0 to 3 amino acid modifications, such as 0, 1, 2, or 3 modifications. In some cases, CDRH1, CDRH2, and CDRH3 comprise the sequence as set forth in SEQ ID NOs: 11, 12, and 13, respectively, each with 0 to 3 amino acid modifications, such as 0, 1, 2, or 3 modifications. In some cases, CDRH1, CDRH2, and CDRH3 comprise the sequence as set forth in SEQ ID NOs: 19, 20, and 21, respectively, each with 0 to 3 amino acid modifications, such as 0, 1, 2, or 3 modifications. In some cases, CDRH1, CDRH2, and CDRH3 comprise the sequence as set forth in SEQ ID NOs: 27, 28, and 29, respectively, each with 0 to 3 amino acid modifications, such as 0, 1, 2, or 3 modifications. In some cases, CDRH1, CDRH2, and CDRH3 comprise the sequence as set forth in SEQ ID NOs: 35, 36, and 37, respectively, each with 0 to 3 amino acid modifications, such as 0, 1, 2, or 3 modifications.

In some embodiments, the light chain further comprises at least one light chain complementarity determining region (CDR). In some embodiments, the light chain CDR comprises a sequence as set forth in any of SEQ ID NO: 6, 7, 8, 14, 15, 16, 22, 23, 24, 30, 31, 32, 38, 39, 40, 67, 68, or 87 to 91, with from 0 to 3 amino acid modifications. In some embodiments, the light chain CDR comprises a sequence as set forth in any of SEQ ID NO: 6, 7, 8, 14, 15, 16, 22, 23, 24, 30, 31, 32, 38, 39, 40, 67, 68, or 87 to 91, with from 0 to 2 amino acid modifications. In some embodiments, the light chain CDR comprises a sequence as set forth in any of SEQ ID NO: 6, 7, 8, 14, 15, 16, 22, 23, 24, 30, 31, 32, 38, 39, 40, 67, 68, or 87 to 91, with from 0 to 1 amino acid modifications.

In some embodiments, the heavy chain comprises a light chain variable region. In some cases, the light chain variable region comprises light chain complementarity determining region 1 (CDRL1), CDRL2, and CDRL3. In some cases, CDRL1, CDRL2, and CDRL3 comprise the sequence as set forth in SEQ ID NOs: 6, 7, and 8, respectively, each with 0 to 3 amino acid modifications, such as 0, 1, 2, or 3 modifications. In some cases, CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some cases, CDRL1, CDRL2, and CDRL3 comprise the sequence as set forth in SEQ ID NOs: 6, 68, and 8, respectively, each with 0 to 3 amino acid modifications, such as 0, 1, 2, or 3 modifications. In some cases, CDRL1, CDRL2, and CDRL3 comprise the sequence as set forth in SEQ ID NOs: 14, 15, and 16, respectively, each with 0 to 3 amino acid modifications, such as 0, 1, 2, or 3 modifications. In some cases, CDRL1, CDRL2, and CDRL3 comprise the sequence as set forth in SEQ ID NOs: 22, 23, and 24, respectively, each with 0 to 3 amino acid modifications, such as 0, 1, 2, or 3 modifications. In some cases, CDRL1, CDRL2, and CDRL3 comprise the sequence as set forth in SEQ ID NOs: 30, 31, and 32, respectively, each with 0 to 3 amino acid modifications, such as 0, 1, 2, or 3 modifications. In some cases, CDRL1, CDRL2, and CDRL3 comprise the sequence as set forth in SEQ ID NOs: 38, 39, and 40, respectively, each with 0 to 3 amino acid modifications, such as 0, 1, 2, or 3 modifications. In some cases, CDRL1, CDRL2, and CDRL3 comprise the sequence as set forth in SEQ ID NOs: 6, 7, and 8, respectively, each with 0 to 2 amino acid modifications. In some cases, CDRL1, CDRL2, and CDRL3 comprise the sequence as set forth in SEQ ID NOs: 6, 67, and 8, respectively, each with 0 to 2 amino acid modifications. In some cases, CDRL1, CDRL2, and CDRL3 comprise the sequence as set forth in SEQ ID NOs: 6, 68, and 8, respectively, each with 0 to 2 amino acid modifications. In some cases, CDRL1, CDRL2, and CDRL3 comprise the sequence as set forth in SEQ ID NOs: 14, 15, and 16, respectively, each with 0 to 2 amino acid modifications. In some cases, CDRL1, CDRL2, and CDRL3 comprise the sequence as set forth in SEQ ID NOs: 22, 23, and 24, respectively, each with 0 to 2 amino acid modifications. In some cases, CDRL1, CDRL2, and CDRL3 comprise the sequence as set forth in SEQ ID NOs: 30, 31, and 32, respectively, each with 0 to 2 amino acid modifications. In some cases, CDRL1, CDRL2, and CDRL3 comprise the sequence as set forth in SEQ ID NOs: 38, 39, and 40, respectively, each with 0 to 2 amino acid modifications. In some cases, CDRL1, CDRL2, and CDRL3 comprise the sequence as set forth in SEQ ID NOs: 14, 15, and 16, respectively, each with 0 to 2 amino acid modifications. In some cases, CDRL1, CDRL2, and CDRL3 comprise the sequence as set forth in SEQ ID NOs: 22, 23, and 24, respectively, each with 0 to 1 amino acid modifications. In some cases, CDRL1, CDRL2, and CDRL3 comprise the sequence as set forth in SEQ ID NOs: 30, 31, and 32, respectively, each with 0 to 1 amino acid modifications. In some cases, CDRL1, CDRL2, and CDRL3 comprise the sequence as set forth in SEQ ID NOs: 38, 39, and 40, respectively, each with 0 to 1 amino acid modifications.

In some embodiments, the antibody provided herein (or antigen-binding fragment, hereafter referred to as “antibody,” to represent the full length antibody or the antigen-binding fragment of the antibody provided herein) comprises a heavy chain that comprises at least one heavy chain CDR as set forth in any of SEQ ID NO: 3, 4, 41, 5, 69, or 70, with from 0 to 3 amino acid modifications, and a light chain that comprises at least one light chain CDR as set forth in any of SEQ ID NO: 6, 7, or 8, with from 0 to 3 amino acid modifications. In some cases, the antibody provided herein comprises a heavy chain that comprises at least one heavy chain CDR as set forth in any of SEQ ID NO: 3, 4, 41, 5, 69, or 70, with from 0 to 3 amino acid modifications, and a light chain that comprises at least one light chain CDR as set forth in any of SEQ ID NO: 6, 67, or 8, with from 0 to 3 amino acid modifications. In some cases, the antibody provided herein comprises a heavy chain that comprises at least one heavy chain CDR as set forth in any of SEQ ID NO: 3, 4, 41, 5, 69, or 70, with from 0 to 3 amino acid modifications, and a light chain that comprises at least one light chain CDR as set forth in any of SEQ ID NO: 6, 68, or 8, with from 0 to 3 amino acid modifications. In some cases, the antibody provided herein comprises a heavy chain that comprises at least one heavy chain CDR as set forth in any of SEQ ID NO: 3, 41, or 70, with from 0 to 3 amino acid modifications, and a light chain that comprises at least one light chain CDR as set forth in any of SEQ ID NO: 6, 67, or 8 with from 0 to 3 amino acid modifications. In some cases, the antibody provided herein comprises a heavy chain that comprises at least one heavy chain CDR as set forth in any of SEQ ID NO: 3, 41, or 69, with from 0 to 3 amino acid modifications, and a light chain that comprises at least one light chain CDR as set forth in any of SEQ ID NO: 6, 67, or 8 with from 0 to 3 amino acid modifications. In some cases, the antibody provided herein comprises a heavy chain that comprises at least one heavy chain CDR as set forth in any of SEQ ID NO: 11, 12, or 13, with from 0 to 3 amino acid modifications, and a light chain that comprises at least one light chain CDR as set forth in any of SEQ ID NO: 14, 15, or 16, with from 0 to 3 amino acid modifications. In some cases, the antibody provided herein comprises a heavy chain that comprises at least one heavy chain CDR as set forth in any of SEQ ID NO: 19, 20, or 21, with from 0 to 3 amino acid modifications, and a light chain that comprises at least one light chain CDR as set forth in any of SEQ ID NO: 22, 23, or 24, with from 0 to 3 amino acid modifications. In some cases, the antibody provided herein comprises a heavy chain that comprises at least one heavy chain CDR as set forth in any of SEQ ID NO: 27, 28, or 29, with from 0 to 3 amino acid modifications, and a light chain that comprises at least one light chain CDR as set forth in any of SEQ ID NO: 30, 31, or 32, with from 0 to 3 amino acid modifications. In some cases, the antibody provided herein comprises a heavy chain that comprises at least one heavy chain CDR as set forth in any of SEQ ID NO: 35, 36, or 37, with from 0 to 3 amino acid modifications, and a light chain that comprises at least one light chain CDR as set forth in any of SEQ ID NO: 38, 39, or 40, with from 0 to 3 amino acid modifications.

In some cases, the antibody provided herein comprises a heavy chain that comprises at least one heavy chain CDR as set forth in any of SEQ ID NO: 3, 4, 41, 5, 69, or 70, with from 0 to 2 amino acid modifications, and a light chain that comprises at least one light chain CDR as set forth in any of SEQ ID NO: 6, 7, or 8, with from 0 to 2 amino acid modifications. In some cases, the antibody provided herein comprises a heavy chain that comprises at least one heavy chain CDR as set forth in any of SEQ ID NO: 3, 4, 41, 5, 69, or 70, with from 0 to 2 amino acid modifications, and a light chain that comprises at least one light chain CDR as set forth in any of SEQ ID NO: 6, 67, or 8, with from 0 to 2 amino acid modifications. In some cases, the antibody provided herein comprises a heavy chain that comprises at least one heavy chain CDR as set forth in any of SEQ ID NO: 3, 4, 41, 5, 69, or 70, with from 0 to 2 amino acid modifications, and a light chain that comprises at least one light chain CDR as set forth in any of SEQ ID NO: 6, 68, or 8, with from 0 to 2 amino acid modifications. In some cases, the antibody provided herein comprises a heavy chain that comprises at least one heavy chain CDR as set forth in any of SEQ ID NO: 11, 12, or 13, with from 0 to 2 amino acid modifications, and a light chain that comprises at least one light chain CDR as set forth in any of SEQ ID NO: 14, 15, or 16, with from 0 to 2 amino acid modifications. In some cases, the antibody provided herein comprises a heavy chain that comprises at least one heavy chain CDR as set forth in any of SEQ ID NO: 19, 20, or 21, with from 0 to 2 amino acid modifications, and a light chain that comprises at least one light chain CDR as set forth in any of SEQ ID NO: 22, 23, or 24, with from 0 to 2 amino acid modifications. In some cases, the antibody provided herein comprises a heavy chain that comprises at least one heavy chain CDR as set forth in any of SEQ ID NO: 27, 28, or 29, with from 0 to 2 amino acid modifications, and a light chain that comprises at least one light chain CDR as set forth in any of SEQ ID NO: 30, 31, or 32, with from 0 to 2 amino acid modifications. In some cases, the antibody provided herein comprises a heavy chain that comprises at least one heavy chain CDR as set forth in any of SEQ ID NO: 35, 36, or 37, with from 0 to 2 amino acid modifications, and a light chain that comprises at least one light chain CDR as set forth in any of SEQ ID NO: 38, 39, or 40, with from 0 to 2 amino acid modifications.

In some cases, the antibody provided herein comprises a heavy chain that comprises at least one heavy chain CDR as set forth in any of SEQ ID NO: 3, 4, 41, 5, 69, or 70, with from 0 to 2 amino acid modifications, and a light chain that comprises at least one light chain CDR as set forth in any of SEQ ID NO: 6, 7, or 8, with from 0 to 1 amino acid modifications. In some cases, the antibody provided herein comprises a heavy chain that comprises at least one heavy chain CDR as set forth in any of SEQ ID NO: 3, 4, 41, 5, 69, or 70, with from 0 to 2 amino acid modifications, and a light chain that comprises at least one light chain CDR as set forth in any of SEQ ID NO: 6, 67, or 8, with from 0 to 1 amino acid modifications. In some cases, the antibody provided herein comprises a heavy chain that comprises at least one heavy chain CDR as set forth in any of SEQ ID NO: 3, 4, 41, 5, 69, or 70, with from 0 to 2 amino acid modifications, and a light chain that comprises at least one light chain CDR as set forth in any of SEQ ID NO: 6, 68, or 8, with from 0 to 1 amino acid modifications. In some cases, the antibody provided herein comprises a heavy chain that comprises at least one heavy chain CDR as set forth in any of SEQ ID NO: 11, 12, or 13, with from 0 to 1 amino acid modifications, and a light chain that comprises at least one light chain CDR as set forth in any of SEQ ID NO: 14, 15, or 16, with from 0 to 1 amino acid modifications. In some cases, the antibody provided herein comprises a heavy chain that comprises at least one heavy chain CDR as set forth in any of SEQ ID NO: 19, 20, or 21, with from 0 to 1 amino acid modifications, and a light chain that comprises at least one light chain CDR as set forth in any of SEQ ID NO: 22, 23, or 24, with from 0 to 1 amino acid modifications. In some cases, the antibody provided herein comprises a heavy chain that comprises at least one heavy chain CDR as set forth in any of SEQ ID NO: 27, 28, or 29, with from 0 to 1 amino acid modifications, and a light chain that comprises at least one light chain CDR as set forth in any of SEQ ID NO: 30, 31, or 32, with from 0 to 1 amino acid modifications. In some cases, the antibody provided herein comprises a heavy chain that comprises at least one heavy chain CDR as set forth in any of SEQ ID NO: 35, 36, or 37, with from 0 to 1 amino acid modifications, and a light chain that comprises at least one light chain CDR as set forth in any of SEQ ID NO: 38, 39, or 40, with from 0 to 1 amino acid modifications.

In some embodiments, the antibody provided herein comprises a heavy chain comprising a heavy chain variable region that further comprises heavy chain complementarity determining region 1 (CDRH1), CDRH2, and CDRH3 and a light chain comprising a light chain variable region that comprises light chain complementarity determining region 1 (CDRL1), CDRL2, and CDRL3, wherein CDRL1, CDRL2, and CDRL3. In some cases, the CDRH1, CDRH2, and CDRH3 comprise the sequence as set forth in SEQ ID NOs: 3, 4, and 5, respectively, each with 0 to 3 amino acid modifications, such as 0, 1, 2, or 3 modifications and the CDRL1, CDRL2, and CDRL3 comprise the sequence as set forth in SEQ ID NOs: 6, 7, and 8, respectively, each with 0 to 3 amino acid modifications, such as 0, 1, 2, or 3 modifications. In some cases, the CDRH1, CDRH2, and CDRH3 comprise the sequence as set forth in SEQ ID NOs: 3, 4, and 5, respectively, each with 0 to 3 amino acid modifications, such as 0, 1, 2, or 3 modifications and the CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some cases, the CDRH1, CDRH2, and CDRH3 comprise the sequence as set forth in SEQ ID NOs: 3, 41, 69, respectively, each with 0 to 3 amino acid modifications, such as 0, 1, 2, or 3 modifications and the CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some cases, the CDRH1, CDRH2, and CDRH3 comprise the sequence as set forth in SEQ ID NOs: 3, 41, 70, respectively, each with 0 to 3 amino acid modifications, such as 0, 1, 2, or 3 modifications and the CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some cases, the CDRH1, CDRH2, and CDRH3 comprise the sequence as set forth in SEQ ID NOs: 3, 4, and 5, respectively, each with 0 to 3 amino acid modifications, such as 0, 1, 2, or 3 modifications and the CDRL1, CDRL2, and CDRL3 comprise the sequence as set forth in SEQ ID NOs: 6, 68, and 8, respectively, each with 0 to 3 amino acid modifications, such as 0, 1, 2, or 3 modifications. In some cases, the CDRH1, CDRH2, and CDRH3 comprise the sequence as set forth in SEQ ID NOs: 3, 41, and 5, respectively, each with 0 to 3 amino acid modifications, such as 0, 1, 2, or 3 modifications, and the CDRL1, CDRL2, and CDRL3 comprise the sequence as set forth in SEQ ID NOs: 6, 7, and 8, respectively, each with 0 to 3 amino acid modifications, such as 0, 1, 2, or 3 modifications. In some cases, the CDRH1, CDRH2, and CDRH3 comprise the sequence as set forth in SEQ ID NOs: 3, 41, and 5, respectively, each with 0 to 3 amino acid modifications, such as 0, 1, 2, or 3 modifications, and the CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some cases, the CDRH1, CDRH2, and CDRH3 comprise the sequence as set forth in SEQ ID NOs: 3, 41, and 5, respectively, each with 0 to 3 amino acid modifications, such as 0, 1, 2, or 3 modifications, and the CDRL1, CDRL2, and CDRL3 comprise the sequence as set forth in SEQ ID NOs: 6, 68, and 8, respectively, each with 0 to 3 amino acid modifications, such as 0, 1, 2, or 3 modifications. In some cases, the CDRH1, CDRH2, and CDRH3 comprise the sequence as set forth in SEQ ID NOs: 11, 12, and 13, respectively, each with 0 to 3 amino acid modifications, such as 0, 1, 2, or 3 modifications, and the CDRL1, CDRL2, and CDRL3 comprise the sequence as set forth in SEQ ID NOs: 14, 15, and 16, respectively, each with 0 to 3 amino acid modifications, such as 0, 1, 2, or 3 modifications. In some cases, the CDRH1, CDRH2, and CDRH3 comprise the sequence as set forth in SEQ ID NOs: 19, 20, and 21, respectively, each with 0 to 3 amino acid modifications, such as 0, 1, 2, or 3 modifications, and the CDRL1, CDRL2, and CDRL3 comprise the sequence as set forth in SEQ ID NOs: 22, 23, and 24, respectively, each with 0 to 3 amino acid modifications, such as 0, 1, 2, or 3 modifications. In some cases, the CDRH1, CDRH2, and CDRH3 comprise the sequence as set forth in SEQ ID NOs: 27, 28, and 29, respectively, each with 0 to 3 amino acid modifications, such as 0, 1, 2, or 3 modifications, and the CDRL1, CDRL2, and CDRL3 comprise the sequence as set forth in SEQ ID NOs: 30, 31, and 32, respectively, each with 0 to 3 amino acid modifications, such as 0, 1, 2, or 3 modifications. In some cases, the CDRH1, CDRH2, and CDRH3 comprise the sequence as set forth in SEQ ID NOs: 35, 36, and 37, respectively, each with 0 to 3 amino acid modifications, such as 0, 1, 2, or 3 modifications, and the CDRL1, CDRL2, and CDRL3 comprise the sequence as set forth in SEQ ID NOs: 38, 39, and 40, respectively, each with 0 to 3 amino acid modifications, such as 0, 1, 2, or 3 modifications.

In some cases, the CDRH1, CDRH2, and CDRH3 comprise the sequence as set forth in SEQ ID NOs: 3, 4, and 5, respectively, each with 0 to 2 amino acid modifications and the CDRL1, CDRL2, and CDRL3 comprise the sequence as set forth in SEQ ID NOs: 6, 7, and 8, respectively, each with 0 to 2 amino acid modifications. In some cases, the CDRH1, CDRH2, and CDRH3 comprise the sequence as set forth in SEQ ID NOs: 3, 4, and 5, respectively, each with 0 to 2 amino acid modifications and the CDRL1, CDRL2, and CDRL3 comprise the sequence as set forth in SEQ ID NOs: 6, 67, and 8, respectively, each with 0 to 2 amino acid modifications. In some cases, the CDRH1, CDRH2, and CDRH3 comprise the sequence as set forth in SEQ ID NOs: 3, 41, 69, respectively, each with 0 to 2 amino acid modifications and the CDRL1, CDRL2, and CDRL3 comprise the sequence as set forth in SEQ ID NOs: 6, 67, and 8, respectively, each with 0 to 2 amino acid modifications. In some cases, the CDRH1, CDRH2, and CDRH3 comprise the sequence as set forth in SEQ ID NOs: 3, 41, 70, respectively, each with 0 to 2 amino acid modifications and the CDRL1, CDRL2, and CDRL3 comprise the sequence as set forth in SEQ ID NOs: 6, 67, and 8, respectively, each with 0 to 2 amino acid modifications. In some cases, the CDRH1, CDRH2, and CDRH3 comprise the sequence as set forth in SEQ ID NOs: 3, 4, and 5, respectively, each with 0 to 2 amino acid modifications and the CDRL1, CDRL2, and CDRL3 comprise the sequence as set forth in SEQ ID NOs: 6, 68, and 8, respectively, each with 0 to 2 amino acid modifications. In some cases, the CDRH1, CDRH2, and CDRH3 comprise the sequence as set forth in SEQ ID NOs: 3, 41, and 5, respectively, each with 0 to 2 amino acid modifications, and the CDRL1, CDRL2, and CDRL3 comprise the sequence as set forth in SEQ ID NOs: 6, 7, and 8, respectively, each with 0 to 2 amino acid modifications. In some cases, the CDRH1, CDRH2, and CDRH3 comprise the sequence as set forth in SEQ ID NOs: 3, 41, and 5, respectively, each with 0 to 2 amino acid modifications, and the CDRL1, CDRL2, and CDRL3 comprise the sequence as set forth in SEQ ID NOs: 6, 67, and 8, respectively, each with 0 to 2 amino acid modifications. In some cases, the CDRH1, CDRH2, and CDRH3 comprise the sequence as set forth in SEQ ID NOs: 3, 41, and 5, respectively, each with 0 to 2 amino acid modifications, and the CDRL1, CDRL2, and CDRL3 comprise the sequence as set forth in SEQ ID NOs: 6, 68, and 8, respectively, each with 0 to 2 amino acid modifications. In some cases, the CDRH1, CDRH2, and CDRH3 comprise the sequence as set forth in SEQ ID NOs: 11, 12, and 13, respectively, each with 0 to 2 amino acid modifications, and the CDRL1, CDRL2, and CDRL3 comprise the sequence as set forth in SEQ ID NOs: 14, 15, and 16, respectively, each with 0 to 2 amino acid modifications. In some cases, the CDRH1, CDRH2, and CDRH3 comprise the sequence as set forth in SEQ ID NOs: 19, 20, and 21, respectively, each with 0 to 2 amino acid modifications, and the CDRL1, CDRL2, and CDRL3 comprise the sequence as set forth in SEQ ID NOs: 22, 23, and 24, respectively, each with 0 to 2 amino acid modifications. In some cases, the CDRH1, CDRH2, and CDRH3 comprise the sequence as set forth in SEQ ID NOs: 27, 28, and 29, respectively, each with 0 to 2 amino acid modifications, and the CDRL1, CDRL2, and CDRL3 comprise the sequence as set forth in SEQ ID NOs: 30, 31, and 32, respectively, each with 0 to 2 amino acid modifications. In some cases, the CDRH1, CDRH2, and CDRH3 comprise the sequence as set forth in SEQ ID NOs: 35, 36, and 37, respectively, each with 0 to 2 amino acid modifications, and the CDRL1, CDRL2, and CDRL3 comprise the sequence as set forth in SEQ ID NOs: 38, 39, and 40, respectively, each with 0 to 2 amino acid modifications.

In some cases, the CDRH1, CDRH2, and CDRH3 comprise the sequence as set forth in SEQ ID NOs: 3, 4, and 5, respectively, each with 0 to 1 amino acid modifications and the CDRL1, CDRL2, and CDRL3 comprise the sequence as set forth in SEQ ID NOs: 6, 7, and 8, respectively, each with 0 to 1 amino acid modifications. In some cases, the CDRH1, CDRH2, and CDRH3 comprise the sequence as set forth in SEQ ID NOs: 3, 4, and 5, respectively, each with 0 to 1 amino acid modifications and the CDRL1, CDRL2, and CDRL3 comprise the sequence as set forth in SEQ ID NOs: 6, 67, and 8, respectively, each with 0 to 1 amino acid modifications. In some cases, the CDRH1, CDRH2, and CDRH3 comprise the sequence as set forth in SEQ ID NOs: 3, 41, 69, respectively, each with 0 to 1 amino acid modifications and the CDRL1, CDRL2, and CDRL3 comprise the sequence as set forth in SEQ ID NOs: 6, 67, and 8, respectively, each with 0 to 1 amino acid modifications. In some cases, the CDRH1, CDRH2, and CDRH3 comprise the sequence as set forth in SEQ ID NOs: 3, 41, 70, respectively, each with 0 to 1 amino acid modifications and the CDRL1, CDRL2, and CDRL3 comprise the sequence as set forth in SEQ ID NOs: 6, 67, and 8, respectively, each with 0 to 1 amino acid modifications. In some cases, the CDRH1, CDRH2, and CDRH3 comprise the sequence as set forth in SEQ ID NOs: 3, 4, and 5, respectively, each with 0 to 1 amino acid modifications and the CDRL1, CDRL2, and CDRL3 comprise the sequence as set forth in SEQ ID NOs: 6, 68, and 8, respectively, each with 0 to 1 amino acid modifications. In some cases, the CDRH1, CDRH2, and CDRH3 comprise the sequence as set forth in SEQ ID NOs: 3, 41, and 5, respectively, each with 0 to 1 amino acid modifications, and the CDRL1, CDRL2, and CDRL3 comprise the sequence as set forth in SEQ ID NOs: 6, 7, and 8, respectively, each with 0 to 1 amino acid modifications. In some cases, the CDRH1, CDRH2, and CDRH3 comprise the sequence as set forth in SEQ ID NOs: 3, 41, and 5, respectively, each with 0 to 1 amino acid modifications, and the CDRL1, CDRL2, and CDRL3 comprise the sequence as set forth in SEQ ID NOs: 6, 67, and 8, respectively, each with 0 to 1 amino acid modifications. In some cases, the CDRH1, CDRH2, and CDRH3 comprise the sequence as set forth in SEQ ID NOs: 3, 41, and 5, respectively, each with 0 to 1 amino acid modifications, and the CDRL1, CDRL2, and CDRL3 comprise the sequence as set forth in SEQ ID NOs: 6, 68, and 8, respectively, each with 0 to 1 amino acid modifications. In some cases, the CDRH1, CDRH2, and CDRH3 comprise the sequence as set forth in SEQ ID NOs: 11, 12, and 13, respectively, each with 0 to 1 amino acid modifications, and the CDRL1, CDRL2, and CDRL3 comprise the sequence as set forth in SEQ ID NOs: 14, 15, and 16, respectively, each with 0 to 1 amino acid modifications. In some cases, the CDRH1, CDRH2, and CDRH3 comprise the sequence as set forth in SEQ ID NOs: 19, 20, and 21, respectively, each with 0 to 1 amino acid modifications, and the CDRL1, CDRL2, and CDRL3 comprise the sequence as set forth in SEQ ID NOs: 22, 23, and 24, respectively, each with 0 to 1 amino acid modifications. In some cases, the CDRH1, CDRH2, and CDRH3 comprise the sequence as set forth in SEQ ID NOs: 27, 28, and 29, respectively, each with 0 to 1 amino acid modifications, and the CDRL1, CDRL2, and CDRL3 comprise the sequence as set forth in SEQ ID NOs: 30, 31, and 32, respectively, each with 0 to 1 amino acid modifications. In some cases, the CDRH1, CDRH2, and CDRH3 comprise the sequence as set forth in SEQ ID NOs: 35, 36, and 37, respectively, each with 0 to 1 amino acid modifications, and the CDRL1, CDRL2, and CDRL3 comprise the sequence as set forth in SEQ ID NOs: 38, 39, and 40, respectively, each with 0 to 1 amino acid modifications.

In some embodiments, the antibody provided herein comprises a heavy chain variable region that comprises an amino acid sequence having at least 90% sequence identity to the amino acid sequence as set forth in SEQ ID NO: 1, 9, 17, 25, 33, 42, 43, 44, 45, 46, 47, 71, 72, or 93. In some embodiments, the antibody provided herein comprises a heavy chain variable region that comprises an amino acid sequence having at least 95% sequence identity to the amino acid sequence as set forth in SEQ ID NO: 1, 9, 17, 25, 33, 42, 43, 44, 45, 46, 47, 71, 72, or 93. In some embodiments, the antibody provided herein comprises a heavy chain variable region that comprises an amino acid sequence having at least 96% sequence identity to the amino acid sequence as set forth in SEQ ID NO: 1, 9, 17, 25, 33, 42, 43, 44, 45, 46, 47, 71, 72, or 93. In some embodiments, the antibody provided herein comprises a heavy chain variable region that comprises an amino acid sequence having at least 97% sequence identity to the amino acid sequence as set forth in SEQ ID NO: 1, 9, 17, 25, 33, 42, 43, 44, 45, 46, 47, 71, 72, or 93. In some embodiments, the antibody provided herein comprises a heavy chain variable region that comprises an amino acid sequence having at least 98% sequence identity to the amino acid sequence as set forth in SEQ ID NO: 1, 9, 17, 25, 33, 42, 43, 44, 45, 46, 47, 71, 72, or 93. In some embodiments, the antibody provided herein comprises a heavy chain variable region that comprises an amino acid sequence having at least 99% sequence identity to the amino acid sequence as set forth in SEQ ID NO: 1, 9, 17, 25, 33, 42, 43, 44, 45, 46, 47, 71, 72, or 93. In some embodiments, the antibody provided herein comprises a heavy chain variable region that comprises an amino acid sequence having 100% sequence identity to the amino acid sequence as set forth in SEQ ID NO: 1, 9, 17, 25, 33, 42, 43, 44, 45, 46, 47, 71, 72, or 93.

In some embodiments, the antibody provided herein comprises a heavy chain variable region that comprises an amino acid sequence as set forth in SEQ ID NO: 1, 9, 17, 25, 33, 42, 43, 44, 45, 46, 47, 71, 72, or 93with from 0 to 3 amino acid modifications. In some embodiments, the antibody provided herein comprises a heavy chain variable region that comprises an amino acid sequence as set forth in SEQ ID NO: 1, 9, 17, 25, 33, 42, 43, 44, 45, 46, 47, 71, 72, or 93with from 0 to 2 amino acid modifications. In some embodiments, the antibody provided herein comprises a heavy chain variable region that comprises an amino acid sequence as set forth in SEQ ID NO: 1, 9, 17, 25, 33, 42, 43, 44, 45, 46, 47, 71, 72, or 93with from 0 to 2 amino acid modifications. In some embodiments, the antibody provided herein comprises a heavy chain variable region that comprises an amino acid sequence as set forth in SEQ ID NO: 1, 9, 17, 25, 33, 42, 43, 44, 45, 46, 47, 71, 72, or 93with from 0 to 1 amino acid modifications. In some embodiments, the antibody provided herein comprises a heavy chain variable region that comprises an amino acid sequence as set forth in SEQ ID NO: 1, 9, 17, 25, 33, 42, 43, 44, 45, 46, 47, 71, 72, or 93.

In some embodiments, the antibody provided herein comprises a light chain variable region that comprises an amino acid sequence having at least 90% sequence identity to the amino acid sequence as set forth in SEQ ID NO: 2, 10, 18, 26, 34, 48, 49, 50, 51, 52, 65, 66, or 94. In some embodiments, the antibody provided herein comprises a light chain variable region that comprises an amino acid sequence having at least 95% sequence identity to the amino acid sequence as set forth in SEQ ID NO: 2, 10, 18, 26, 34, 48, 49, 50, 51, 52, 65, 66, or 94. In some embodiments, the antibody provided herein comprises a light chain variable region that comprises an amino acid sequence having at least 96% sequence identity to the amino acid sequence as set forth in SEQ ID NO: 2, 10, 18, 26, 34, 48, 49, 50, 51, 52, 65, 66, or 94. In some embodiments, the antibody provided herein comprises a light chain variable region that comprises an amino acid sequence having at least 97% sequence identity to the amino acid sequence as set forth in SEQ ID NO: 2, 10, 18, 26, 34, 48, 49, 50, 51, 52, 65, 66, or 94. In some embodiments, the antibody provided herein comprises a light chain variable region that comprises an amino acid sequence having at least 98% sequence identity to the amino acid sequence as set forth in SEQ ID NO: 2, 10, 18, 26, 34, 48, 49, 50, 51, 52, 65, 66, or 94. In some embodiments, the antibody provided herein comprises a light chain variable region that comprises an amino acid sequence having at least 99% sequence identity to the amino acid sequence as set forth in SEQ ID NO: 2, 10, 18, 26, 34, 48, 49, 50, 51, 52, 65, 66, or 94. In some embodiments, the antibody provided herein comprises a light chain variable region that comprises an amino acid sequence having at 100% sequence identity to the amino acid sequence as set forth in SEQ ID NO: 2, 10, 18, 26, 34, 48, 49, 50, 51, 52, 65, 66, or 94.

In some embodiments, the antibody provided herein comprises a light chain variable region that comprises an amino acid sequence as set forth in SEQ ID NO: 2, 10, 18, 26, 34, 48, 49, 50, 51, 52, 65, 66, or 94, with from 0 to 3 amino acid modifications. In some embodiments, the antibody provided herein comprises a light chain variable region that comprises an amino acid sequence as set forth in SEQ ID NO: 2, 10, 18, 26, 34, 48, 49, 50, 51, 52, 65, 66, or 94, with from 0 to 2 amino acid modifications. In some embodiments, the antibody provided herein comprises a light chain variable region that comprises an amino acid sequence as set forth in SEQ ID NO: 2, 10, 18, 26, 34, 48, 49, 50, 51, 52, 65, 66, or 94, with from 0 to 1 amino acid modifications. In some embodiments, the antibody provided herein comprises a light chain variable region that comprises an amino acid sequence as set forth in SEQ ID NO: 2, 10, 18, 26, 34, 48, 49, 50, 51, 52, 65, 66, or 94.

In some embodiments, the antibody provided herein comprises a heavy chain variable region comprising an amino acid sequence having at least 90% sequence identity to the amino acid sequence as set forth in SEQ ID NO: 1, and a light chain variable region comprising an amino acid sequence having at least 90% sequence identity to the amino acid sequence as set forth in SEQ ID NO: 2. In some cases, the antibody provided herein comprises a heavy chain variable region comprising an amino acid sequence having at least 95% sequence identity to the amino acid sequence as set forth in SEQ ID NO: 1, and a light chain variable region comprising an amino acid sequence having at least 95% sequence identity to the amino acid sequence as set forth in SEQ ID NO: 2. In some cases, the antibody provided herein comprises a heavy chain variable region comprising an amino acid sequence having at least 96% sequence identity to the amino acid sequence as set forth in SEQ ID NO: 1, and a light chain variable region comprising an amino acid sequence having at least 96% sequence identity to the amino acid sequence as set forth in SEQ ID NO: 2. In some cases, the antibody provided herein comprises a heavy chain variable region comprising an amino acid sequence having at least 97% sequence identity to the amino acid sequence as set forth in SEQ ID NO: 1, and a light chain variable region comprising an amino acid sequence having at least 97% sequence identity to the amino acid sequence as set forth in SEQ ID NO: 2. In some cases, the antibody provided herein comprises a heavy chain variable region comprising an amino acid sequence having at least 98% sequence identity to the amino acid sequence as set forth in SEQ ID NO: 1, and a light chain variable region comprising an amino acid sequence having at least 98% sequence identity to the amino acid sequence as set forth in SEQ ID NO: 2. In some cases, the antibody provided herein comprises a heavy chain variable region comprising an amino acid sequence having at least 99% sequence identity to the amino acid sequence as set forth in SEQ ID NO: 1, and a light chain variable region comprising an amino acid sequence having at least 99% sequence identity to the amino acid sequence as set forth in SEQ ID NO: 2. In some cases, the antibody provided herein comprises a heavy chain variable region comprising an amino acid sequence having 100% sequence identity to the amino acid sequence as set forth in SEQ ID NO: 1, and a light chain variable region comprising an amino acid sequence having 100% sequence identity to the amino acid sequence as set forth in SEQ ID NO: 2.

In some embodiments, the antibody provided herein comprises a heavy chain variable region comprising an amino acid sequence having at least 90% sequence identity to the amino acid sequence as set forth in SEQ ID NO: 9, and a light chain variable region comprising an amino acid sequence having at least 90% sequence identity to the amino acid sequence as set forth in SEQ ID NO: 10. In some cases, the antibody provided herein comprises a heavy chain variable region comprising an amino acid sequence having at least 95% sequence identity to the amino acid sequence as set forth in SEQ ID NO: 9, and a light chain variable region comprising an amino acid sequence having at least 95% sequence identity to the amino acid sequence as set forth in SEQ ID NO: 10. In some cases, the antibody provided herein comprises a heavy chain variable region comprising an amino acid sequence having at least 96% sequence identity to the amino acid sequence as set forth in SEQ ID NO: 9, and a light chain variable region comprising an amino acid sequence having at least 96% sequence identity to the amino acid sequence as set forth in SEQ ID NO: 10. In some cases, the antibody provided herein comprises a heavy chain variable region comprising an amino acid sequence having at least 97% sequence identity to the amino acid sequence as set forth in SEQ ID NO: 9, and a light chain variable region comprising an amino acid sequence having at least 97% sequence identity to the amino acid sequence as set forth in SEQ ID NO: 10. In some cases, the antibody provided herein comprises a heavy chain variable region comprising an amino acid sequence having at least 98% sequence identity to the amino acid sequence as set forth in SEQ ID NO: 9, and a light chain variable region comprising an amino acid sequence having at least 98% sequence identity to the amino acid sequence as set forth in SEQ ID NO: 10. In some cases, the antibody provided herein comprises a heavy chain variable region comprising an amino acid sequence having at least 99% sequence identity to the amino acid sequence as set forth in SEQ ID NO: 9, and a light chain variable region comprising an amino acid sequence having at least 99% sequence identity to the amino acid sequence as set forth in SEQ ID NO: 10. In some cases, the antibody provided herein comprises a heavy chain variable region comprising an amino acid sequence having 100% sequence identity to the amino acid sequence as set forth in SEQ ID NO: 9, and a light chain variable region comprising an amino acid sequence having 100% sequence identity to the amino acid sequence as set forth in SEQ ID NO: 10.

In some embodiments, the antibody provided herein comprises a heavy chain variable region comprising an amino acid sequence having at least 90% sequence identity to the amino acid sequence as set forth in SEQ ID NO: 17, and a light chain variable region comprising an amino acid sequence having at least 90% sequence identity to the amino acid sequence as set forth in SEQ ID NO: 18. In some cases, the antibody provided herein comprises a heavy chain variable region comprising an amino acid sequence having at least 95% sequence identity to the amino acid sequence as set forth in SEQ ID NO:17, and a light chain variable region comprising an amino acid sequence having at least 95% sequence identity to the amino acid sequence as set forth in SEQ ID NO: 18. In some cases, the antibody provided herein comprises a heavy chain variable region comprising an amino acid sequence having at least 96% sequence identity to the amino acid sequence as set forth in SEQ ID NO: 17, and a light chain variable region comprising an amino acid sequence having at least 96% sequence identity to the amino acid sequence as set forth in SEQ ID NO: 18. In some cases, the antibody provided herein comprises a heavy chain variable region comprising an amino acid sequence having at least 97% sequence identity to the amino acid sequence as set forth in SEQ ID NO: 17, and a light chain variable region comprising an amino acid sequence having at least 97% sequence identity to the amino acid sequence as set forth in SEQ ID NO: 18. In some cases, the antibody provided herein comprises a heavy chain variable region comprising an amino acid sequence having at least 98% sequence identity to the amino acid sequence as set forth in SEQ ID NO: 17, and a light chain variable region comprising an amino acid sequence having at least 98% sequence identity to the amino acid sequence as set forth in SEQ ID NO: 18. In some cases, the antibody provided herein comprises a heavy chain variable region comprising an amino acid sequence having at least 99% sequence identity to the amino acid sequence as set forth in SEQ ID NO: 17, and a light chain variable region comprising an amino acid sequence having at least 99% sequence identity to the amino acid sequence as set forth in SEQ ID NO: 18. In some cases, the antibody provided herein comprises a heavy chain variable region comprising an amino acid sequence having 100% sequence identity to the amino acid sequence as set forth in SEQ ID NO: 17, and a light chain variable region comprising an amino acid sequence having 100% sequence identity to the amino acid sequence as set forth in SEQ ID NO: 18.

In some embodiments, the antibody provided herein comprises a heavy chain variable region comprising an amino acid sequence having at least 90% sequence identity to the amino acid sequence as set forth in SEQ ID NO: 25, and a light chain variable region comprising an amino acid sequence having at least 90% sequence identity to the amino acid sequence as set forth in SEQ ID NO: 26. In some cases, the antibody provided herein comprises a heavy chain variable region comprising an amino acid sequence having at least 95% sequence identity to the amino acid sequence as set forth in SEQ ID NO: 25, and a light chain variable region comprising an amino acid sequence having at least 95% sequence identity to the amino acid sequence as set forth in SEQ ID NO: 26. In some cases, the antibody provided herein comprises a heavy chain variable region comprising an amino acid sequence having at least 96% sequence identity to the amino acid sequence as set forth in SEQ ID NO: 25, and a light chain variable region comprising an amino acid sequence having at least 96% sequence identity to the amino acid sequence as set forth in SEQ ID NO: 26. In some cases, the antibody provided herein comprises a heavy chain variable region comprising an amino acid sequence having at least 97% sequence identity to the amino acid sequence as set forth in SEQ ID NO: 25, and a light chain variable region comprising an amino acid sequence having at least 97% sequence identity to the amino acid sequence as set forth in SEQ ID NO: 26. In some cases, the antibody provided herein comprises a heavy chain variable region comprising an amino acid sequence having at least 98% sequence identity to the amino acid sequence as set forth in SEQ ID NO: 25, and a light chain variable region comprising an amino acid sequence having at least 98% sequence identity to the amino acid sequence as set forth in SEQ ID NO: 26. In some cases, the antibody provided herein comprises a heavy chain variable region comprising an amino acid sequence having at least 99% sequence identity to the amino acid sequence as set forth in SEQ ID NO: 25, and a light chain variable region comprising an amino acid sequence having at least 99% sequence identity to the amino acid sequence as set forth in SEQ ID NO: 26. In some cases, the antibody provided herein comprises a heavy chain variable region comprising an amino acid sequence having 100% sequence identity to the amino acid sequence as set forth in SEQ ID NO: 25, and a light chain variable region comprising an amino acid sequence having 100% sequence identity to the amino acid sequence as set forth in SEQ ID NO: 26.

In some embodiments, the antibody provided herein comprises a heavy chain variable region comprising an amino acid sequence having at least 90% sequence identity to the amino acid sequence as set forth in SEQ ID NO: 33, and a light chain variable region comprising an amino acid sequence having at least 90% sequence identity to the amino acid sequence as set forth in SEQ ID NO: 34. In some cases, the antibody provided herein comprises a heavy chain variable region comprising an amino acid sequence having at least 95% sequence identity to the amino acid sequence as set forth in SEQ ID NO: 33, and a light chain variable region comprising an amino acid sequence having at least 95% sequence identity to the amino acid sequence as set forth in SEQ ID NO: 34. In some cases, the antibody provided herein comprises a heavy chain variable region comprising an amino acid sequence having at least 96% sequence identity to the amino acid sequence as set forth in SEQ ID NO: 33, and a light chain variable region comprising an amino acid sequence having at least 96% sequence identity to the amino acid sequence as set forth in SEQ ID NO: 34. In some cases, the antibody provided herein comprises a heavy chain variable region comprising an amino acid sequence having at least 97% sequence identity to the amino acid sequence as set forth in SEQ ID NO: 33, and a light chain variable region comprising an amino acid sequence having at least 97% sequence identity to the amino acid sequence as set forth in SEQ ID NO: 34. In some cases, the antibody provided herein comprises a heavy chain variable region comprising an amino acid sequence having at least 98% sequence identity to the amino acid sequence as set forth in SEQ ID NO: 33, and a light chain variable region comprising an amino acid sequence having at least 98% sequence identity to the amino acid sequence as set forth in SEQ ID NO: 34. In some cases, the antibody provided herein comprises a heavy chain variable region comprising an amino acid sequence having at least 99% sequence identity to the amino acid sequence as set forth in SEQ ID NO: 33, and a light chain variable region comprising an amino acid sequence having at least 99% sequence identity to the amino acid sequence as set forth in SEQ ID NO: 34. In some cases, the antibody provided herein comprises a heavy chain variable region comprising an amino acid sequence having 100% sequence identity to the amino acid sequence as set forth in SEQ ID NO: 33, and a light chain variable region comprising an amino acid sequence having 100% sequence identity to the amino acid sequence as set forth in SEQ ID NO: 34.

In some embodiments, the antibody provided herein comprises a heavy chain variable region comprising an amino acid sequence having at least 90% sequence identity to the amino acid sequence as set forth in any one of SEQ ID NOs: 1, 42, 43, 44, 45, 46, 47, 71, 72, or 93 and a light chain variable region comprising an amino acid sequence having at least 90% sequence identity to the amino acid sequence as set forth in SEQ ID NO: 2, 48, 49, 50, 51, 52, 65, 66, or 94. In some cases, the antibody provided herein comprises a heavy chain variable region comprising an amino acid sequence having at least 95% sequence identity to the amino acid sequence as set forth in any one of SEQ ID NOs: 1, 42, 43, 44, 45, 46, 47, 71, 72, or 93 and a light chain variable region comprising an amino acid sequence having at least 95% sequence identity to the amino acid sequence as set forth in SEQ ID NO: 2, 48, 49, 50, 51, 52, 65, 66, or 94. In some cases, the antibody provided herein comprises a heavy chain variable region comprising an amino acid sequence having at least 96% sequence identity to the amino acid sequence as set forth in any one of SEQ ID NOs: 1, 42, 43, 44, 45, 46, 47, 71, 72, or 93 and a light chain variable region comprising an amino acid sequence having at least 96% sequence identity to the amino acid sequence as set forth in SEQ ID NO: 2, 48, 49, 50, 51, 52, 65, 66, or 94. In some cases, the antibody provided herein comprises a heavy chain variable region comprising an amino acid sequence having at least 97% sequence identity to the amino acid sequence as set forth in any one of SEQ ID NOs: 1, 42, 43, 44, 45, 46, 47, 71, 72, or 93 and a light chain variable region comprising an amino acid sequence having at least 97% sequence identity to the amino acid sequence as set forth in SEQ ID NO: 2, 48, 49, 50, 51, 52, 65, 66, or 94. In some cases, the antibody provided herein comprises a heavy chain variable region comprising an amino acid sequence having at least 98% sequence identity to the amino acid sequence as set forth in any one of SEQ ID NOs: 1, 42, 43, 44, 45, 46, 47, 71, 72, or 93 and a light chain variable region comprising an amino acid sequence having at least 98% sequence identity to the amino acid sequence as set forth in SEQ ID NO: 2, 48, 49, 50, 51, 52, 65, 66, or 94. In some cases, the antibody provided herein comprises a heavy chain variable region comprising an amino acid sequence having at least 99% sequence identity to the amino acid sequence as set forth in any one of SEQ ID NOs: 1, 42, 43, 44, 45, 46, 47, 71, 72, or 93 and a light chain variable region comprising an amino acid sequence having at least 99% sequence identity to the amino acid sequence as set forth in SEQ ID NO: 2, 48, 49, 50, 51, 52, 65, 66, or 94. In some cases, the antibody provided herein comprises a heavy chain variable region comprising an amino acid sequence having 100% sequence identity to the amino acid sequence as set forth in any one of SEQ ID NOs: 1, 42, 43, 44, 45, 46, 47, 71, 72, or 93 and a light chain variable region comprising an amino acid sequence having 100% sequence identity to the amino acid sequence as set forth in SEQ ID NO: 2, 48, 49, 50, 51, 52, 65, 66, or 94.

In some cases, the antibody provided herein comprises a heavy chain variable region comprising an amino acid sequence having at least 90% sequence identity to the amino acid sequence as set forth in any one of SEQ ID NOs: 42, 43, 44, 45, 46, 47, 71, 72, or 93 and a light chain variable region comprising an amino acid sequence having at least 90% sequence identity to the amino acid sequence as set forth in SEQ ID NOS: 48, 49, 50, 51, 52, 65, 66, or 94. In some cases, the antibody provided herein comprises a heavy chain variable region comprising an amino acid sequence having at least 95% sequence identity to the amino acid sequence as set forth in any one of SEQ ID NOs: 42, 43, 44, 45, 46, 47, 71, 72, or 93 and a light chain variable region comprising an amino acid sequence having at least 95% sequence identity to the amino acid sequence as set forth in SEQ ID NOS: 48, 49, 50, 51, 52, 65, 66, or 94. In some cases, the antibody provided herein comprises a heavy chain variable region comprising an amino acid sequence having at least 96% sequence identity to the amino acid sequence as set forth in any one of SEQ ID NOs: 42, 43, 44, 45, 46, 47, 71, 72, or 93 and a light chain variable region comprising an amino acid sequence having at least 96% sequence identity to the amino acid sequence as set forth in SEQ ID NOS: 48, 49, 50, 51, 52, 65, 66, or 94. In some cases, the antibody provided herein comprises a heavy chain variable region comprising an amino acid sequence having at least 97% sequence identity to the amino acid sequence as set forth in any one of SEQ ID NOs: 42, 43, 44, 45, 46, 47, 71, 72, or 93 and a light chain variable region comprising an amino acid sequence having at least 97% sequence identity to the amino acid sequence as set forth in SEQ ID NOS: 48, 49, 50, 51, 52, 65, 66, or 94. In some cases, the antibody provided herein comprises a heavy chain variable region comprising an amino acid sequence having at least 98% sequence identity to the amino acid sequence as set forth in any one of SEQ ID NOs: 42, 43, 44, 45, 46, 47, 71, 72, or 93 and a light chain variable region comprising an amino acid sequence having at least 98% sequence identity to the amino acid sequence as set forth in SEQ ID NOS: 48, 49, 50, 51, 52, 65, 66, or 94. In some cases, the antibody provided herein comprises a heavy chain variable region comprising an amino acid sequence having at least 99% sequence identity to the amino acid sequence as set forth in any one of SEQ ID NOs: 42, 43, 44, 45, 46, 47, 71, 72, or 93 and a light chain variable region comprising an amino acid sequence having 100% sequence identity to the amino acid sequence as set forth in SEQ ID NOS: 48, 49, 50, 51, 52, 65, 66, or 94.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 1, and a light chain variable region comprising an amino acid sequence having at least 90%, 95%, 96%, 97%, 98%, 99%, or 100% sequence identity to the amino acid sequence as set forth SEQ ID NO: 2. In some cases, the antibody provided herein comprises a heavy chain variable region comprising an amino acid sequence having at least 95% sequence identity to the amino acid sequence as set forth SEQ ID NO: 1, and a light chain variable region comprising an amino acid sequence having at least 95% sequence identity to the amino acid sequence as set forth SEQ ID NO: 2. In some cases, the antibody provided herein comprises a heavy chain variable region comprising an amino acid sequence having at least 97% sequence identity to the amino acid sequence as set forth SEQ ID NO: 1, and a light chain variable region comprising an amino acid sequence having at least 97% sequence identity to the amino acid sequence as set forth SEQ ID NO: 2. In some cases, the antibody provided herein comprises a heavy chain variable region comprising an amino acid sequence having at least 99% sequence identity to the amino acid sequence as set forth SEQ ID NO: 1, and a light chain variable region comprising an amino acid sequence having at least 99% sequence identity to the amino acid sequence as set forth SEQ ID NO: 2. In some cases, the antibody provided herein comprises a heavy chain variable region comprising an amino acid sequence having 100% sequence identity to the amino acid sequence as set forth SEQ ID NO: 1, and a light chain variable region comprising an amino acid sequence having 100% sequence identity to the amino acid sequence as set forth SEQ ID NO: 2. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 1, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 2. In some cases, the antibody provided herein comprises a heavy chain variable region comprising an amino acid sequence having less than or equal to 10 total substitutions, deletions, and/or insertions in the amino acid sequence as set forth SEQ ID NO: 1, and a light chain variable region comprising an amino acid sequence having less than or equal to 10 total substitutions, deletions, and/or insertions in the amino acid sequence as set forth SEQ ID NO: 2. In some cases, the antibody provided herein comprises a heavy chain variable region comprising an amino acid sequence having less than or equal to 7 total substitutions, deletions, and/or insertions in the amino acid sequence as set forth SEQ ID NO: 1, and a light chain variable region comprising an amino acid sequence having less than or equal to 7 total substitutions, deletions, and/or insertions in the amino acid sequence as set forth SEQ ID NO: 2. In some cases, the antibody provided herein comprises a heavy chain variable region comprising an amino acid sequence having less than or equal to 5 total substitutions, deletions, and/or insertions in the amino acid sequence as set forth SEQ ID NO: 1, and a light chain variable region comprising an amino acid sequence having less than or equal to 5 total substitutions, deletions, and/or insertions in the amino acid sequence as set forth SEQ ID NO: 2. In some cases, the antibody provided herein comprises a heavy chain variable region comprising an amino acid sequence having less than or equal to 3 total substitutions, deletions, and/or insertions in the amino acid sequence as set forth SEQ ID NO: 1, and a light chain variable region comprising an amino acid sequence having less than or equal to 3 total substitutions, deletions, and/or insertions in the amino acid sequence as set forth SEQ ID NO: 2. In some cases, the antibody provided herein comprises a heavy chain variable region comprising an amino acid sequence having less than or equal to 2 total substitutions, deletions, and/or insertions in the amino acid sequence as set forth SEQ ID NO: 1, and a light chain variable region comprising an amino acid sequence having less than or equal to 2 total substitutions, deletions, and/or insertions in the amino acid sequence as set forth SEQ ID NO: 2. In some cases, the antibody provided herein comprises a heavy chain variable region comprising an amino acid sequence having less than or equal to 1 total substitutions, deletions, and/or insertions in the amino acid sequence as set forth SEQ ID NO: 1, and a light chain variable region comprising an amino acid sequence having less than or equal to 1 total substitutions, deletions, and/or insertions in the amino acid sequence as set forth SEQ ID NO: 2.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 1, and a light chain variable region comprising an amino acid sequence having at least 90%, 95%, 96%, 97%, 98%, 99%, or 100% sequence identity to the amino acid sequence as set forth SEQ ID NO: 48. In some cases, the antibody provided herein comprises a heavy chain variable region comprising an amino acid sequence having at least 95% sequence identity to the amino acid sequence as set forth SEQ ID NO: 1, and a light chain variable region comprising an amino acid sequence having at least 95% sequence identity to the amino acid sequence as set forth SEQ ID NO: 48. In some cases, the antibody provided herein comprises a heavy chain variable region comprising an amino acid sequence having at least 97% sequence identity to the amino acid sequence as set forth SEQ ID NO: 1, and a light chain variable region comprising an amino acid sequence having at least 97% sequence identity to the amino acid sequence as set forth SEQ ID NO: 48. In some cases, the antibody provided herein comprises a heavy chain variable region comprising an amino acid sequence having at least 99% sequence identity to the amino acid sequence as set forth SEQ ID NO: 1, and a light chain variable region comprising an amino acid sequence having at least 99% sequence identity to the amino acid sequence as set forth SEQ ID NO: 48. In some cases, the antibody provided herein comprises a heavy chain variable region comprising an amino acid sequence having 100% sequence identity to the amino acid sequence as set forth SEQ ID NO: 1, and a light chain variable region comprising an amino acid sequence having 100% sequence identity to the amino acid sequence as set forth SEQ ID NO: 48. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 1, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 48. In some cases, the antibody provided herein comprises a heavy chain variable region comprising an amino acid sequence having less than or equal to 10 total substitutions, deletions, and/or insertions in the amino acid sequence as set forth SEQ ID NO: 1, and a light chain variable region comprising an amino acid sequence having less than or equal to 10 total substitutions, deletions, and/or insertions in the amino acid sequence as set forth SEQ ID NO: 48. In some cases, the antibody provided herein comprises a heavy chain variable region comprising an amino acid sequence having less than or equal to 7 total substitutions, deletions, and/or insertions in the amino acid sequence as set forth SEQ ID NO: 1, and a light chain variable region comprising an amino acid sequence having less than or equal to 7 total substitutions, deletions, and/or insertions in the amino acid sequence as set forth SEQ ID NO: 48. In some cases, the antibody provided herein comprises a heavy chain variable region comprising an amino acid sequence having less than or equal to 5 total substitutions, deletions, and/or insertions in the amino acid sequence as set forth SEQ ID NO: 1, and a light chain variable region comprising an amino acid sequence having less than or equal to 5 total substitutions, deletions, and/or insertions in the amino acid sequence as set forth SEQ ID NO: 48. In some cases, the antibody provided herein comprises a heavy chain variable region comprising an amino acid sequence having less than or equal to 3 total substitutions, deletions, and/or insertions in the amino acid sequence as set forth SEQ ID NO: 1, and a light chain variable region comprising an amino acid sequence having less than or equal to 3 total substitutions, deletions, and/or insertions in the amino acid sequence as set forth SEQ ID NO: 48. In some cases, the antibody provided herein comprises a heavy chain variable region comprising an amino acid sequence having less than or equal to 2 total substitutions, deletions, and/or insertions in the amino acid sequence as set forth SEQ ID NO: 1, and a light chain variable region comprising an amino acid sequence having less than or equal to 2 total substitutions, deletions, and/or insertions in the amino acid sequence as set forth SEQ ID NO: 48. In some cases, the antibody provided herein comprises a heavy chain variable region comprising an amino acid sequence having less than or equal to 1 total substitutions, deletions, and/or insertions in the amino acid sequence as set forth SEQ ID NO: 1, and a light chain variable region comprising an amino acid sequence having less than or equal to 1 total substitutions, deletions, and/or insertions in the amino acid sequence as set forth SEQ ID NO: 48

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 1, and a light chain variable region comprising an amino acid sequence having at least 90%, 95%, 96%, 97%, 98%, 99%, or 100% sequence identity to the amino acid sequence as set forth SEQ ID NO: 49. In some cases, the antibody provided herein comprises a heavy chain variable region comprising an amino acid sequence having at least 95% sequence identity to the amino acid sequence as set forth SEQ ID NO: 1, and a light chain variable region comprising an amino acid sequence having at least 95% sequence identity to the amino acid sequence as set forth SEQ ID NO: 49. In some cases, the antibody provided herein comprises a heavy chain variable region comprising an amino acid sequence having at least 97% sequence identity to the amino acid sequence as set forth SEQ ID NO: 1, and a light chain variable region comprising an amino acid sequence having at least 97% sequence identity to the amino acid sequence as set forth SEQ ID NO: 49. In some cases, the antibody provided herein comprises a heavy chain variable region comprising an amino acid sequence having at least 99% sequence identity to the amino acid sequence as set forth SEQ ID NO: 1, and a light chain variable region comprising an amino acid sequence having at least 99% sequence identity to the amino acid sequence as set forth SEQ ID NO: 49. In some cases, the antibody provided herein comprises a heavy chain variable region comprising an amino acid sequence having 100% sequence identity to the amino acid sequence as set forth SEQ ID NO: 1, and a light chain variable region comprising an amino acid sequence having 100% sequence identity to the amino acid sequence as set forth SEQ ID NO: 49. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 1, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 49. In some cases, the antibody provided herein comprises a heavy chain variable region comprising an amino acid sequence having less than or equal to 10 total substitutions, deletions, and/or insertions in the amino acid sequence as set forth SEQ ID NO: 1, and a light chain variable region comprising an amino acid sequence having less than or equal to 10 total substitutions, deletions, and/or insertions in the amino acid sequence as set forth SEQ ID NO: 49. In some cases, the antibody provided herein comprises a heavy chain variable region comprising an amino acid sequence having less than or equal to 7 total substitutions, deletions, and/or insertions in the amino acid sequence as set forth SEQ ID NO: 1, and a light chain variable region comprising an amino acid sequence having less than or equal to 7 total substitutions, deletions, and/or insertions in the amino acid sequence as set forth SEQ ID NO: 49. In some cases, the antibody provided herein comprises a heavy chain variable region comprising an amino acid sequence having less than or equal to 5 total substitutions, deletions, and/or insertions in the amino acid sequence as set forth SEQ ID NO: 1, and a light chain variable region comprising an amino acid sequence having less than or equal to 5 total substitutions, deletions, and/or insertions in the amino acid sequence as set forth SEQ ID NO: 49. In some cases, the antibody provided herein comprises a heavy chain variable region comprising an amino acid sequence having less than or equal to 3 total substitutions, deletions, and/or insertions in the amino acid sequence as set forth SEQ ID NO: 1, and a light chain variable region comprising an amino acid sequence having less than or equal to 3 total substitutions, deletions, and/or insertions in the amino acid sequence as set forth SEQ ID NO: 49. In some cases, the antibody provided herein comprises a heavy chain variable region comprising an amino acid sequence having less than or equal to 2 total substitutions, deletions, and/or insertions in the amino acid sequence as set forth SEQ ID NO: 1, and a light chain variable region comprising an amino acid sequence having less than or equal to 2 total substitutions, deletions, and/or insertions in the amino acid sequence as set forth SEQ ID NO: 49. In some cases, the antibody provided herein comprises a heavy chain variable region comprising an amino acid sequence having less than or equal to 1 total substitutions, deletions, and/or insertions in the amino acid sequence as set forth SEQ ID NO: 1, and a light chain variable region comprising an amino acid sequence having less than or equal to 1 total substitutions, deletions, and/or insertions in the amino acid sequence as set forth SEQ ID NO: 49.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 1, and a light chain variable region comprising an amino acid sequence having at least 90%, 95%, 96%, 97%, 98%, 99%, or 100% sequence identity to the amino acid sequence as set forth SEQ ID NO: 50. In some cases, the antibody provided herein comprises a heavy chain variable region comprising an amino acid sequence having at least 95% sequence identity to the amino acid sequence as set forth SEQ ID NO: 1, and a light chain variable region comprising an amino acid sequence having at least 95% sequence identity to the amino acid sequence as set forth SEQ ID NO: 50. In some cases, the antibody provided herein comprises a heavy chain variable region comprising an amino acid sequence having at least 97% sequence identity to the amino acid sequence as set forth SEQ ID NO: 1, and a light chain variable region comprising an amino acid sequence having at least 97% sequence identity to the amino acid sequence as set forth SEQ ID NO: 50. In some cases, the antibody provided herein comprises a heavy chain variable region comprising an amino acid sequence having at least 99% sequence identity to the amino acid sequence as set forth SEQ ID NO: 1, and a light chain variable region comprising an amino acid sequence having at least 99% sequence identity to the amino acid sequence as set forth SEQ ID NO: 50. In some cases, the antibody provided herein comprises a heavy chain variable region comprising an amino acid sequence having 100% sequence identity to the amino acid sequence as set forth SEQ ID NO: 1, and a light chain variable region comprising an amino acid sequence having 100% sequence identity to the amino acid sequence as set forth SEQ ID NO: 50. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 1, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 50. In some cases, the antibody provided herein comprises a heavy chain variable region comprising an amino acid sequence having less than or equal to 10 total substitutions, deletions, and/or insertions in the amino acid sequence as set forth SEQ ID NO: 1, and a light chain variable region comprising an amino acid sequence having less than or equal to 10 total substitutions, deletions, and/or insertions in the amino acid sequence as set forth SEQ ID NO: 50. In some cases, the antibody provided herein comprises a heavy chain variable region comprising an amino acid sequence having less than or equal to 7 total substitutions, deletions, and/or insertions in the amino acid sequence as set forth SEQ ID NO: 1, and a light chain variable region comprising an amino acid sequence having less than or equal to 7 total substitutions, deletions, and/or insertions in the amino acid sequence as set forth SEQ ID NO: 50. In some cases, the antibody provided herein comprises a heavy chain variable region comprising an amino acid sequence having less than or equal to 5 total substitutions, deletions, and/or insertions in the amino acid sequence as set forth SEQ ID NO: 1, and a light chain variable region comprising an amino acid sequence having less than or equal to 5 total substitutions, deletions, and/or insertions in the amino acid sequence as set forth SEQ ID NO: 50. In some cases, the antibody provided herein comprises a heavy chain variable region comprising an amino acid sequence having less than or equal to 3 total substitutions, deletions, and/or insertions in the amino acid sequence as set forth SEQ ID NO: 1, and a light chain variable region comprising an amino acid sequence having less than or equal to 3 total substitutions, deletions, and/or insertions in the amino acid sequence as set forth SEQ ID NO: 50. In some cases, the antibody provided herein comprises a heavy chain variable region comprising an amino acid sequence having less than or equal to 2 total substitutions, deletions, and/or insertions in the amino acid sequence as set forth SEQ ID NO: 1, and a light chain variable region comprising an amino acid sequence having less than or equal to 2 total substitutions, deletions, and/or insertions in the amino acid sequence as set forth SEQ ID NO: 50. In some cases, the antibody provided herein comprises a heavy chain variable region comprising an amino acid sequence having less than or equal to 1 total substitutions, deletions, and/or insertions in the amino acid sequence as set forth SEQ ID NO: 1, and a light chain variable region comprising an amino acid sequence having less than or equal to 1 total substitutions, deletions, and/or insertions in the amino acid sequence as set forth SEQ ID NO: 50.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 1, and a light chain variable region comprising an amino acid sequence having at least 90%, 95%, 96%, 97%, 98%, 99%, or 100% sequence identity to the amino acid sequence as set forth SEQ ID NO: 51. In some cases, the antibody provided herein comprises a heavy chain variable region comprising an amino acid sequence having at least 95% sequence identity to the amino acid sequence as set forth SEQ ID NO: 1, and a light chain variable region comprising an amino acid sequence having at least 95% sequence identity to the amino acid sequence as set forth SEQ ID NO: 51. In some cases, the antibody provided herein comprises a heavy chain variable region comprising an amino acid sequence having at least 97% sequence identity to the amino acid sequence as set forth SEQ ID NO: 1, and a light chain variable region comprising an amino acid sequence having at least 97% sequence identity to the amino acid sequence as set forth SEQ ID NO: 51. In some cases, the antibody provided herein comprises a heavy chain variable region comprising an amino acid sequence having at least 99% sequence identity to the amino acid sequence as set forth SEQ ID NO: 1, and a light chain variable region comprising an amino acid sequence having at least 99% sequence identity to the amino acid sequence as set forth SEQ ID NO: 51. In some cases, the antibody provided herein comprises a heavy chain variable region comprising an amino acid sequence having 100% sequence identity to the amino acid sequence as set forth SEQ ID NO: 1, and a light chain variable region comprising an amino acid sequence having 100% sequence identity to the amino acid sequence as set forth SEQ ID NO: 51. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 1, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 51. In some cases, the antibody provided herein comprises a heavy chain variable region comprising an amino acid sequence having less than or equal to 10 total substitutions, deletions, and/or insertions in the amino acid sequence as set forth SEQ ID NO: 1, and a light chain variable region comprising an amino acid sequence having less than or equal to 10 total substitutions, deletions, and/or insertions in the amino acid sequence as set forth SEQ ID NO: 51. In some cases, the antibody provided herein comprises a heavy chain variable region comprising an amino acid sequence having less than or equal to 7 total substitutions, deletions, and/or insertions in the amino acid sequence as set forth SEQ ID NO: 1, and a light chain variable region comprising an amino acid sequence having less than or equal to 7 total substitutions, deletions, and/or insertions in the amino acid sequence as set forth SEQ ID NO: 51. In some cases, the antibody provided herein comprises a heavy chain variable region comprising an amino acid sequence having less than or equal to 5 total substitutions, deletions, and/or insertions in the amino acid sequence as set forth SEQ ID NO: 1, and a light chain variable region comprising an amino acid sequence having less than or equal to 5 total substitutions, deletions, and/or insertions in the amino acid sequence as set forth SEQ ID NO: 51. In some cases, the antibody provided herein comprises a heavy chain variable region comprising an amino acid sequence having less than or equal to 3 total substitutions, deletions, and/or insertions in the amino acid sequence as set forth SEQ ID NO: 1, and a light chain variable region comprising an amino acid sequence having less than or equal to 3 total substitutions, deletions, and/or insertions in the amino acid sequence as set forth SEQ ID NO: 51. In some cases, the antibody provided herein comprises a heavy chain variable region comprising an amino acid sequence having less than or equal to 2 total substitutions, deletions, and/or insertions in the amino acid sequence as set forth SEQ ID NO: 1, and a light chain variable region comprising an amino acid sequence having less than or equal to 2 total substitutions, deletions, and/or insertions in the amino acid sequence as set forth SEQ ID NO: 51. In some cases, the antibody provided herein comprises a heavy chain variable region comprising an amino acid sequence having less than or equal to 1 total substitutions, deletions, and/or insertions in the amino acid sequence as set forth SEQ ID NO: 1, and a light chain variable region comprising an amino acid sequence having less than or equal to 1 total substitutions, deletions, and/or insertions in the amino acid sequence as set forth SEQ ID NO: 51

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 1, and a light chain variable region comprising an amino acid sequence having at least 90%, 95%, 96%, 97%, 98%, 99%, or 100% sequence identity to the amino acid sequence as set forth SEQ ID NO: 52. In some cases, the antibody provided herein comprises a heavy chain variable region comprising an amino acid sequence having at least 95% sequence identity to the amino acid sequence as set forth SEQ ID NO: 1, and a light chain variable region comprising an amino acid sequence having at least 95% sequence identity to the amino acid sequence as set forth SEQ ID NO: 52. In some cases, the antibody provided herein comprises a heavy chain variable region comprising an amino acid sequence having at least 97% sequence identity to the amino acid sequence as set forth SEQ ID NO: 1, and a light chain variable region comprising an amino acid sequence having at least 97% sequence identity to the amino acid sequence as set forth SEQ ID NO: 52. In some cases, the antibody provided herein comprises a heavy chain variable region comprising an amino acid sequence having at least 99% sequence identity to the amino acid sequence as set forth SEQ ID NO: 1, and a light chain variable region comprising an amino acid sequence having at least 99% sequence identity to the amino acid sequence as set forth SEQ ID NO: 52. In some cases, the antibody provided herein comprises a heavy chain variable region comprising an amino acid sequence having 100% sequence identity to the amino acid sequence as set forth SEQ ID NO: 1, and a light chain variable region comprising an amino acid sequence having 100% sequence identity to the amino acid sequence as set forth SEQ ID NO: 52. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 1, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 52. In some cases, the antibody provided herein comprises a heavy chain variable region comprising an amino acid sequence having less than or equal to 10 total substitutions, deletions, and/or insertions in the amino acid sequence as set forth SEQ ID NO: 1, and a light chain variable region comprising an amino acid sequence having less than or equal to 10 total substitutions, deletions, and/or insertions in the amino acid sequence as set forth SEQ ID NO: 52. In some cases, the antibody provided herein comprises a heavy chain variable region comprising an amino acid sequence having less than or equal to 7 total substitutions, deletions, and/or insertions in the amino acid sequence as set forth SEQ ID NO: 1, and a light chain variable region comprising an amino acid sequence having less than or equal to 7 total substitutions, deletions, and/or insertions in the amino acid sequence as set forth SEQ ID NO: 52. In some cases, the antibody provided herein comprises a heavy chain variable region comprising an amino acid sequence having less than or equal to 5 total substitutions, deletions, and/or insertions in the amino acid sequence as set forth SEQ ID NO: 1, and a light chain variable region comprising an amino acid sequence having less than or equal to 5 total substitutions, deletions, and/or insertions in the amino acid sequence as set forth SEQ ID NO: 52. In some cases, the antibody provided herein comprises a heavy chain variable region comprising an amino acid sequence having less than or equal to 3 total substitutions, deletions, and/or insertions in the amino acid sequence as set forth SEQ ID NO: 1, and a light chain variable region comprising an amino acid sequence having less than or equal to 3 total substitutions, deletions, and/or insertions in the amino acid sequence as set forth SEQ ID NO: 52. In some cases, the antibody provided herein comprises a heavy chain variable region comprising an amino acid sequence having less than or equal to 2 total substitutions, deletions, and/or insertions in the amino acid sequence as set forth SEQ ID NO: 1, and a light chain variable region comprising an amino acid sequence having less than or equal to 2 total substitutions, deletions, and/or insertions in the amino acid sequence as set forth SEQ ID NO: 52. In some cases, the antibody provided herein comprises a heavy chain variable region comprising an amino acid sequence having less than or equal to 1 total substitutions, deletions, and/or insertions in the amino acid sequence as set forth SEQ ID NO: 1, and a light chain variable region comprising an amino acid sequence having less than or equal to 1 total substitutions, deletions, and/or insertions in the amino acid sequence as set forth SEQ ID NO: 52.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 1, and a light chain variable region comprising an amino acid sequence having at least 90%, 95%, 96%, 97%, 98%, 99%, or 100% sequence identity to the amino acid sequence as set forth SEQ ID NO: 65. In some cases, the antibody provided herein comprises a heavy chain variable region comprising an amino acid sequence having at least 95% sequence identity to the amino acid sequence as set forth SEQ ID NO: 1, and a light chain variable region comprising an amino acid sequence having at least 95% sequence identity to the amino acid sequence as set forth SEQ ID NO: 65. In some cases, the antibody provided herein comprises a heavy chain variable region comprising an amino acid sequence having at least 97% sequence identity to the amino acid sequence as set forth SEQ ID NO: 1, and a light chain variable region comprising an amino acid sequence having at least 97% sequence identity to the amino acid sequence as set forth SEQ ID NO: 65. In some cases, the antibody provided herein comprises a heavy chain variable region comprising an amino acid sequence having at least 99% sequence identity to the amino acid sequence as set forth SEQ ID NO: 1, and a light chain variable region comprising an amino acid sequence having at least 99% sequence identity to the amino acid sequence as set forth SEQ ID NO: 65. In some cases, the antibody provided herein comprises a heavy chain variable region comprising an amino acid sequence having 100% sequence identity to the amino acid sequence as set forth SEQ ID NO: 1, and a light chain variable region comprising an amino acid sequence having 100% sequence identity to the amino acid sequence as set forth SEQ ID NO: 65. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 1, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 65. In some cases, the antibody provided herein comprises a heavy chain variable region comprising an amino acid sequence having less than or equal to 10 total substitutions, deletions, and/or insertions in the amino acid sequence as set forth SEQ ID NO: 1, and a light chain variable region comprising an amino acid sequence having less than or equal to 10 total substitutions, deletions, and/or insertions in the amino acid sequence as set forth SEQ ID NO: 65. In some cases, the antibody provided herein comprises a heavy chain variable region comprising an amino acid sequence having less than or equal to 7 total substitutions, deletions, and/or insertions in the amino acid sequence as set forth SEQ ID NO: 1, and a light chain variable region comprising an amino acid sequence having less than or equal to 7 total substitutions, deletions, and/or insertions in the amino acid sequence as set forth SEQ ID NO: 65. In some cases, the antibody provided herein comprises a heavy chain variable region comprising an amino acid sequence having less than or equal to 5 total substitutions, deletions, and/or insertions in the amino acid sequence as set forth SEQ ID NO: 1, and a light chain variable region comprising an amino acid sequence having less than or equal to 5 total substitutions, deletions, and/or insertions in the amino acid sequence as set forth SEQ ID NO: 65. In some cases, the antibody provided herein comprises a heavy chain variable region comprising an amino acid sequence having less than or equal to 3 total substitutions, deletions, and/or insertions in the amino acid sequence as set forth SEQ ID NO: 1, and a light chain variable region comprising an amino acid sequence having less than or equal to 3 total substitutions, deletions, and/or insertions in the amino acid sequence as set forth SEQ ID NO: 65. In some cases, the antibody provided herein comprises a heavy chain variable region comprising an amino acid sequence having less than or equal to 2 total substitutions, deletions, and/or insertions in the amino acid sequence as set forth SEQ ID NO: 1, and a light chain variable region comprising an amino acid sequence having less than or equal to 2 total substitutions, deletions, and/or insertions in the amino acid sequence as set forth SEQ ID NO: 65. In some cases, the antibody provided herein comprises a heavy chain variable region comprising an amino acid sequence having less than or equal to 1 total substitutions, deletions, and/or insertions in the amino acid sequence as set forth SEQ ID NO: 1, and a light chain variable region comprising an amino acid sequence having less than or equal to 1 total substitutions, deletions, and/or insertions in the amino acid sequence as set forth SEQ ID NO: 65.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 1, and a light chain variable region comprising an amino acid sequence having at least 90%, 95%, 96%, 97%, 98%, 99%, or 100% sequence identity to the amino acid sequence as set forth SEQ ID NO: 66. In some cases, the antibody provided herein comprises a heavy chain variable region comprising an amino acid sequence having at least 95% sequence identity to the amino acid sequence as set forth SEQ ID NO: 1, and a light chain variable region comprising an amino acid sequence having at least 95% sequence identity to the amino acid sequence as set forth SEQ ID NO: 66. In some cases, the antibody provided herein comprises a heavy chain variable region comprising an amino acid sequence having at least 97% sequence identity to the amino acid sequence as set forth SEQ ID NO: 1, and a light chain variable region comprising an amino acid sequence having at least 97% sequence identity to the amino acid sequence as set forth SEQ ID NO: 66. In some cases, the antibody provided herein comprises a heavy chain variable region comprising an amino acid sequence having at least 99% sequence identity to the amino acid sequence as set forth SEQ ID NO: 1, and a light chain variable region comprising an amino acid sequence having at least 99% sequence identity to the amino acid sequence as set forth SEQ ID NO: 66. In some cases, the antibody provided herein comprises a heavy chain variable region comprising an amino acid sequence having 100% sequence identity to the amino acid sequence as set forth SEQ ID NO: 1, and a light chain variable region comprising an amino acid sequence having 100% sequence identity to the amino acid sequence as set forth SEQ ID NO: 66. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 1, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 66. In some cases, the antibody provided herein comprises a heavy chain variable region comprising an amino acid sequence having less than or equal to 10 total substitutions, deletions, and/or insertions in the amino acid sequence as set forth SEQ ID NO: 1, and a light chain variable region comprising an amino acid sequence having less than or equal to 10 total substitutions, deletions, and/or insertions in the amino acid sequence as set forth SEQ ID NO: 66. In some cases, the antibody provided herein comprises a heavy chain variable region comprising an amino acid sequence having less than or equal to 7 total substitutions, deletions, and/or insertions in the amino acid sequence as set forth SEQ ID NO: 1, and a light chain variable region comprising an amino acid sequence having less than or equal to 7 total substitutions, deletions, and/or insertions in the amino acid sequence as set forth SEQ ID NO: 66. In some cases, the antibody provided herein comprises a heavy chain variable region comprising an amino acid sequence having less than or equal to 5 total substitutions, deletions, and/or insertions in the amino acid sequence as set forth SEQ ID NO: 1, and a light chain variable region comprising an amino acid sequence having less than or equal to 5 total substitutions, deletions, and/or insertions in the amino acid sequence as set forth SEQ ID NO: 66. In some cases, the antibody provided herein comprises a heavy chain variable region comprising an amino acid sequence having less than or equal to 3 total substitutions, deletions, and/or insertions in the amino acid sequence as set forth SEQ ID NO: 1, and a light chain variable region comprising an amino acid sequence having less than or equal to 3 total substitutions, deletions, and/or insertions in the amino acid sequence as set forth SEQ ID NO: 66. In some cases, the antibody provided herein comprises a heavy chain variable region comprising an amino acid sequence having less than or equal to 2 total substitutions, deletions, and/or insertions in the amino acid sequence as set forth SEQ ID NO: 1, and a light chain variable region comprising an amino acid sequence having less than or equal to 2 total substitutions, deletions, and/or insertions in the amino acid sequence as set forth SEQ ID NO: 66. In some cases, the antibody provided herein comprises a heavy chain variable region comprising an amino acid sequence having less than or equal to 1 total substitutions, deletions, and/or insertions in the amino acid sequence as set forth SEQ ID NO: 1, and a light chain variable region comprising an amino acid sequence having less than or equal to 1 total substitutions, deletions, and/or insertions in the amino acid sequence as set forth SEQ ID NO: 66.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 42, and a light chain variable region comprising an amino acid sequence having at least 90%, 95%, 96%, 97%, 98%, 99%, or 100% sequence identity to the amino acid sequence as set forth SEQ ID NO: 2. In some cases, the antibody provided herein comprises a heavy chain variable region comprising an amino acid sequence having at least 95% sequence identity to the amino acid sequence as set forth SEQ ID NO: 42, and a light chain variable region comprising an amino acid sequence having at least 95% sequence identity to the amino acid sequence as set forth SEQ ID NO: 2. In some cases, the antibody provided herein comprises a heavy chain variable region comprising an amino acid sequence having at least 97% sequence identity to the amino acid sequence as set forth SEQ ID NO: 42, and a light chain variable region comprising an amino acid sequence having at least 97% sequence identity to the amino acid sequence as set forth SEQ ID NO: 2. In some cases, the antibody provided herein comprises a heavy chain variable region comprising an amino acid sequence having at least 99% sequence identity to the amino acid sequence as set forth SEQ ID NO: 42, and a light chain variable region comprising an amino acid sequence having at least 99% sequence identity to the amino acid sequence as set forth SEQ ID NO: 2. In some cases, the antibody provided herein comprises a heavy chain variable region comprising an amino acid sequence having 100% sequence identity to the amino acid sequence as set forth SEQ ID NO: 42, and a light chain variable region comprising an amino acid sequence having 100% sequence identity to the amino acid sequence as set forth SEQ ID NO: 2. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 42, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 2. In some cases, the antibody provided herein comprises a heavy chain variable region comprising an amino acid sequence having less than or equal to 10 total substitutions, deletions, and/or insertions in the amino acid sequence as set forth SEQ ID NO: 42, and a light chain variable region comprising an amino acid sequence having less than or equal to 10 total substitutions, deletions, and/or insertions in the amino acid sequence as set forth SEQ ID NO: 2. In some cases, the antibody provided herein comprises a heavy chain variable region comprising an amino acid sequence having less than or equal to 7 total substitutions, deletions, and/or insertions in the amino acid sequence as set forth SEQ ID NO: 42, and a light chain variable region comprising an amino acid sequence having less than or equal to 7 total substitutions, deletions, and/or insertions in the amino acid sequence as set forth SEQ ID NO: 2. In some cases, the antibody provided herein comprises a heavy chain variable region comprising an amino acid sequence having less than or equal to 5 total substitutions, deletions, and/or insertions in the amino acid sequence as set forth SEQ ID NO: 42, and a light chain variable region comprising an amino acid sequence having less than or equal to 5 total substitutions, deletions, and/or insertions in the amino acid sequence as set forth SEQ ID NO: 2. In some cases, the antibody provided herein comprises a heavy chain variable region comprising an amino acid sequence having less than or equal to 3 total substitutions, deletions, and/or insertions in the amino acid sequence as set forth SEQ ID NO: 42, and a light chain variable region comprising an amino acid sequence having less than or equal to 3 total substitutions, deletions, and/or insertions in the amino acid sequence as set forth SEQ ID NO: 2. In some cases, the antibody provided herein comprises a heavy chain variable region comprising an amino acid sequence having less than or equal to 2 total substitutions, deletions, and/or insertions in the amino acid sequence as set forth SEQ ID NO: 42, and a light chain variable region comprising an amino acid sequence having less than or equal to 2 total substitutions, deletions, and/or insertions in the amino acid sequence as set forth SEQ ID NO: 2. In some cases, the antibody provided herein comprises a heavy chain variable region comprising an amino acid sequence having less than or equal to 1 total substitutions, deletions, and/or insertions in the amino acid sequence as set forth SEQ ID NO: 42, and a light chain variable region comprising an amino acid sequence having less than or equal to 1 total substitutions, deletions, and/or insertions in the amino acid sequence as set forth SEQ ID NO: 2.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 42, and a light chain variable region comprising an amino acid sequence having at least 90%, 95%, 96%, 97%, 98%, 99%, or 100% sequence identity to the amino acid sequence as set forth SEQ ID NO: 48. In some cases, the antibody provided herein comprises a heavy chain variable region comprising an amino acid sequence having at least 95% sequence identity to the amino acid sequence as set forth SEQ ID NO: 42, and a light chain variable region comprising an amino acid sequence having at least 95% sequence identity to the amino acid sequence as set forth SEQ ID NO: 48. In some cases, the antibody provided herein comprises a heavy chain variable region comprising an amino acid sequence having at least 97% sequence identity to the amino acid sequence as set forth SEQ ID NO: 42, and a light chain variable region comprising an amino acid sequence having at least 97% sequence identity to the amino acid sequence as set forth SEQ ID NO: 48. In some cases, the antibody provided herein comprises a heavy chain variable region comprising an amino acid sequence having at least 99% sequence identity to the amino acid sequence as set forth SEQ ID NO: 42, and a light chain variable region comprising an amino acid sequence having at least 99% sequence identity to the amino acid sequence as set forth SEQ ID NO: 48. In some cases, the antibody provided herein comprises a heavy chain variable region comprising an amino acid sequence having 100% sequence identity to the amino acid sequence as set forth SEQ ID NO: 42, and a light chain variable region comprising an amino acid sequence having 100% sequence identity to the amino acid sequence as set forth SEQ ID NO: 48. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 42, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 48. In some cases, the antibody provided herein comprises a heavy chain variable region comprising an amino acid sequence having less than or equal to 10 total substitutions, deletions, and/or insertions in the amino acid sequence as set forth SEQ ID NO: 42, and a light chain variable region comprising an amino acid sequence having less than or equal to 10 total substitutions, deletions, and/or insertions in the amino acid sequence as set forth SEQ ID NO: 48. In some cases, the antibody provided herein comprises a heavy chain variable region comprising an amino acid sequence having less than or equal to 7 total substitutions, deletions, and/or insertions in the amino acid sequence as set forth SEQ ID NO: 42, and a light chain variable region comprising an amino acid sequence having less than or equal to 7 total substitutions, deletions, and/or insertions in the amino acid sequence as set forth SEQ ID NO: 48. In some cases, the antibody provided herein comprises a heavy chain variable region comprising an amino acid sequence having less than or equal to 5 total substitutions, deletions, and/or insertions in the amino acid sequence as set forth SEQ ID NO: 42, and a light chain variable region comprising an amino acid sequence having less than or equal to 5 total substitutions, deletions, and/or insertions in the amino acid sequence as set forth SEQ ID NO: 48. In some cases, the antibody provided herein comprises a heavy chain variable region comprising an amino acid sequence having less than or equal to 3 total substitutions, deletions, and/or insertions in the amino acid sequence as set forth SEQ ID NO: 42, and a light chain variable region comprising an amino acid sequence having less than or equal to 3 total substitutions, deletions, and/or insertions in the amino acid sequence as set forth SEQ ID NO: 48. In some cases, the antibody provided herein comprises a heavy chain variable region comprising an amino acid sequence having less than or equal to 2 total substitutions, deletions, and/or insertions in the amino acid sequence as set forth SEQ ID NO: 42, and a light chain variable region comprising an amino acid sequence having less than or equal to 2 total substitutions, deletions, and/or insertions in the amino acid sequence as set forth SEQ ID NO: 48. In some cases, the antibody provided herein comprises a heavy chain variable region comprising an amino acid sequence having less than or equal to 1 total substitutions, deletions, and/or insertions in the amino acid sequence as set forth SEQ ID NO: 42, and a light chain variable region comprising an amino acid sequence having less than or equal to 1 total substitutions, deletions, and/or insertions in the amino acid sequence as set forth SEQ ID NO: 48

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 42, and a light chain variable region comprising an amino acid sequence having at least 90%, 95%, 96%, 97%, 98%, 99%, or 100% sequence identity to the amino acid sequence as set forth SEQ ID NO: 49. In some cases, the antibody provided herein comprises a heavy chain variable region comprising an amino acid sequence having at least 95% sequence identity to the amino acid sequence as set forth SEQ ID NO: 42, and a light chain variable region comprising an amino acid sequence having at least 95% sequence identity to the amino acid sequence as set forth SEQ ID NO: 49. In some cases, the antibody provided herein comprises a heavy chain variable region comprising an amino acid sequence having at least 97% sequence identity to the amino acid sequence as set forth SEQ ID NO: 42, and a light chain variable region comprising an amino acid sequence having at least 97% sequence identity to the amino acid sequence as set forth SEQ ID NO: 49. In some cases, the antibody provided herein comprises a heavy chain variable region comprising an amino acid sequence having at least 99% sequence identity to the amino acid sequence as set forth SEQ ID NO: 42, and a light chain variable region comprising an amino acid sequence having at least 99% sequence identity to the amino acid sequence as set forth SEQ ID NO: 49. In some cases, the antibody provided herein comprises a heavy chain variable region comprising an amino acid sequence having 100% sequence identity to the amino acid sequence as set forth SEQ ID NO: 42, and a light chain variable region comprising an amino acid sequence having 100% sequence identity to the amino acid sequence as set forth SEQ ID NO: 49. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 42, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 49. In some cases, the antibody provided herein comprises a heavy chain variable region comprising an amino acid sequence having less than or equal to 10 total substitutions, deletions, and/or insertions in the amino acid sequence as set forth SEQ ID NO: 42, and a light chain variable region comprising an amino acid sequence having less than or equal to 10 total substitutions, deletions, and/or insertions in the amino acid sequence as set forth SEQ ID NO: 49. In some cases, the antibody provided herein comprises a heavy chain variable region comprising an amino acid sequence having less than or equal to 7 total substitutions, deletions, and/or insertions in the amino acid sequence as set forth SEQ ID NO: 42, and a light chain variable region comprising an amino acid sequence having less than or equal to 7 total substitutions, deletions, and/or insertions in the amino acid sequence as set forth SEQ ID NO: 49. In some cases, the antibody provided herein comprises a heavy chain variable region comprising an amino acid sequence having less than or equal to 5 total substitutions, deletions, and/or insertions in the amino acid sequence as set forth SEQ ID NO: 42, and a light chain variable region comprising an amino acid sequence having less than or equal to 5 total substitutions, deletions, and/or insertions in the amino acid sequence as set forth SEQ ID NO: 49. In some cases, the antibody provided herein comprises a heavy chain variable region comprising an amino acid sequence having less than or equal to 3 total substitutions, deletions, and/or insertions in the amino acid sequence as set forth SEQ ID NO: 42, and a light chain variable region comprising an amino acid sequence having less than or equal to 3 total substitutions, deletions, and/or insertions in the amino acid sequence as set forth SEQ ID NO: 49. In some cases, the antibody provided herein comprises a heavy chain variable region comprising an amino acid sequence having less than or equal to 2 total substitutions, deletions, and/or insertions in the amino acid sequence as set forth SEQ ID NO: 42, and a light chain variable region comprising an amino acid sequence having less than or equal to 2 total substitutions, deletions, and/or insertions in the amino acid sequence as set forth SEQ ID NO: 49. In some cases, the antibody provided herein comprises a heavy chain variable region comprising an amino acid sequence having less than or equal to 1 total substitutions, deletions, and/or insertions in the amino acid sequence as set forth SEQ ID NO: 42, and a light chain variable region comprising an amino acid sequence having less than or equal to 1 total substitutions, deletions, and/or insertions in the amino acid sequence as set forth SEQ ID NO: 49.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 42, and a light chain variable region comprising an amino acid sequence having at least 90%, 95%, 96%, 97%, 98%, 99%, or 100% sequence identity to the amino acid sequence as set forth SEQ ID NO: 50. In some cases, the antibody provided herein comprises a heavy chain variable region comprising an amino acid sequence having at least 95% sequence identity to the amino acid sequence as set forth SEQ ID NO: 42, and a light chain variable region comprising an amino acid sequence having at least 95% sequence identity to the amino acid sequence as set forth SEQ ID NO: 50. In some cases, the antibody provided herein comprises a heavy chain variable region comprising an amino acid sequence having at least 97% sequence identity to the amino acid sequence as set forth SEQ ID NO: 42, and a light chain variable region comprising an amino acid sequence having at least 97% sequence identity to the amino acid sequence as set forth SEQ ID NO: 50. In some cases, the antibody provided herein comprises a heavy chain variable region comprising an amino acid sequence having at least 99% sequence identity to the amino acid sequence as set forth SEQ ID NO: 42, and a light chain variable region comprising an amino acid sequence having at least 99% sequence identity to the amino acid sequence as set forth SEQ ID NO: 50. In some cases, the antibody provided herein comprises a heavy chain variable region comprising an amino acid sequence having 100% sequence identity to the amino acid sequence as set forth SEQ ID NO: 42, and a light chain variable region comprising an amino acid sequence having 100% sequence identity to the amino acid sequence as set forth SEQ ID NO: 50. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 42, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 50. In some cases, the antibody provided herein comprises a heavy chain variable region comprising an amino acid sequence having less than or equal to 10 total substitutions, deletions, and/or insertions in the amino acid sequence as set forth SEQ ID NO: 42, and a light chain variable region comprising an amino acid sequence having less than or equal to 10 total substitutions, deletions, and/or insertions in the amino acid sequence as set forth SEQ ID NO: 50. In some cases, the antibody provided herein comprises a heavy chain variable region comprising an amino acid sequence having less than or equal to 7 total substitutions, deletions, and/or insertions in the amino acid sequence as set forth SEQ ID NO: 42, and a light chain variable region comprising an amino acid sequence having less than or equal to 7 total substitutions, deletions, and/or insertions in the amino acid sequence as set forth SEQ ID NO: 50. In some cases, the antibody provided herein comprises a heavy chain variable region comprising an amino acid sequence having less than or equal to 5 total substitutions, deletions, and/or insertions in the amino acid sequence as set forth SEQ ID NO: 42, and a light chain variable region comprising an amino acid sequence having less than or equal to 5 total substitutions, deletions, and/or insertions in the amino acid sequence as set forth SEQ ID NO: 50. In some cases, the antibody provided herein comprises a heavy chain variable region comprising an amino acid sequence having less than or equal to 3 total substitutions, deletions, and/or insertions in the amino acid sequence as set forth SEQ ID NO: 42, and a light chain variable region comprising an amino acid sequence having less than or equal to 3 total substitutions, deletions, and/or insertions in the amino acid sequence as set forth SEQ ID NO: 50. In some cases, the antibody provided herein comprises a heavy chain variable region comprising an amino acid sequence having less than or equal to 2 total substitutions, deletions, and/or insertions in the amino acid sequence as set forth SEQ ID NO: 42, and a light chain variable region comprising an amino acid sequence having less than or equal to 2 total substitutions, deletions, and/or insertions in the amino acid sequence as set forth SEQ ID NO: 50. In some cases, the antibody provided herein comprises a heavy chain variable region comprising an amino acid sequence having less than or equal to 1 total substitutions, deletions, and/or insertions in the amino acid sequence as set forth SEQ ID NO: 42, and a light chain variable region comprising an amino acid sequence having less than or equal to 1 total substitutions, deletions, and/or insertions in the amino acid sequence as set forth SEQ ID NO: 50.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 42, and a light chain variable region comprising an amino acid sequence having at least 90%, 95%, 96%, 97%, 98%, 99%, or 100% sequence identity to the amino acid sequence as set forth SEQ ID NO: 51. In some cases, the antibody provided herein comprises a heavy chain variable region comprising an amino acid sequence having at least 95% sequence identity to the amino acid sequence as set forth SEQ ID NO: 42, and a light chain variable region comprising an amino acid sequence having at least 95% sequence identity to the amino acid sequence as set forth SEQ ID NO: 51. In some cases, the antibody provided herein comprises a heavy chain variable region comprising an amino acid sequence having at least 97% sequence identity to the amino acid sequence as set forth SEQ ID NO: 42, and a light chain variable region comprising an amino acid sequence having at least 97% sequence identity to the amino acid sequence as set forth SEQ ID NO: 51. In some cases, the antibody provided herein comprises a heavy chain variable region comprising an amino acid sequence having at least 99% sequence identity to the amino acid sequence as set forth SEQ ID NO: 42, and a light chain variable region comprising an amino acid sequence having at least 99% sequence identity to the amino acid sequence as set forth SEQ ID NO: 51. In some cases, the antibody provided herein comprises a heavy chain variable region comprising an amino acid sequence having 100% sequence identity to the amino acid sequence as set forth SEQ ID NO: 42, and a light chain variable region comprising an amino acid sequence having 100% sequence identity to the amino acid sequence as set forth SEQ ID NO: 51. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 42, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 51. In some cases, the antibody provided herein comprises a heavy chain variable region comprising an amino acid sequence having less than or equal to 10 total substitutions, deletions, and/or insertions in the amino acid sequence as set forth SEQ ID NO: 42, and a light chain variable region comprising an amino acid sequence having less than or equal to 10 total substitutions, deletions, and/or insertions in the amino acid sequence as set forth SEQ ID NO: 51. In some cases, the antibody provided herein comprises a heavy chain variable region comprising an amino acid sequence having less than or equal to 7 total substitutions, deletions, and/or insertions in the amino acid sequence as set forth SEQ ID NO: 42, and a light chain variable region comprising an amino acid sequence having less than or equal to 7 total substitutions, deletions, and/or insertions in the amino acid sequence as set forth SEQ ID NO: 51. In some cases, the antibody provided herein comprises a heavy chain variable region comprising an amino acid sequence having less than or equal to 5 total substitutions, deletions, and/or insertions in the amino acid sequence as set forth SEQ ID NO: 42, and a light chain variable region comprising an amino acid sequence having less than or equal to 5 total substitutions, deletions, and/or insertions in the amino acid sequence as set forth SEQ ID NO: 51. In some cases, the antibody provided herein comprises a heavy chain variable region comprising an amino acid sequence having less than or equal to 3 total substitutions, deletions, and/or insertions in the amino acid sequence as set forth SEQ ID NO: 42, and a light chain variable region comprising an amino acid sequence having less than or equal to 3 total substitutions, deletions, and/or insertions in the amino acid sequence as set forth SEQ ID NO: 51. In some cases, the antibody provided herein comprises a heavy chain variable region comprising an amino acid sequence having less than or equal to 2 total substitutions, deletions, and/or insertions in the amino acid sequence as set forth SEQ ID NO: 42, and a light chain variable region comprising an amino acid sequence having less than or equal to 2 total substitutions, deletions, and/or insertions in the amino acid sequence as set forth SEQ ID NO: 51. In some cases, the antibody provided herein comprises a heavy chain variable region comprising an amino acid sequence having less than or equal to 1 total substitutions, deletions, and/or insertions in the amino acid sequence as set forth SEQ ID NO: 42, and a light chain variable region comprising an amino acid sequence having less than or equal to 1 total substitutions, deletions, and/or insertions in the amino acid sequence as set forth SEQ ID NO: 51

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 42, and a light chain variable region comprising an amino acid sequence having at least 90%, 95%, 96%, 97%, 98%, 99%, or 100% sequence identity to the amino acid sequence as set forth SEQ ID NO: 52. In some cases, the antibody provided herein comprises a heavy chain variable region comprising an amino acid sequence having at least 95% sequence identity to the amino acid sequence as set forth SEQ ID NO: 42, and a light chain variable region comprising an amino acid sequence having at least 95% sequence identity to the amino acid sequence as set forth SEQ ID NO: 52. In some cases, the antibody provided herein comprises a heavy chain variable region comprising an amino acid sequence having at least 97% sequence identity to the amino acid sequence as set forth SEQ ID NO: 42, and a light chain variable region comprising an amino acid sequence having at least 97% sequence identity to the amino acid sequence as set forth SEQ ID NO: 52. In some cases, the antibody provided herein comprises a heavy chain variable region comprising an amino acid sequence having at least 99% sequence identity to the amino acid sequence as set forth SEQ ID NO: 42, and a light chain variable region comprising an amino acid sequence having at least 99% sequence identity to the amino acid sequence as set forth SEQ ID NO: 52. In some cases, the antibody provided herein comprises a heavy chain variable region comprising an amino acid sequence having 100% sequence identity to the amino acid sequence as set forth SEQ ID NO: 42, and a light chain variable region comprising an amino acid sequence having 100% sequence identity to the amino acid sequence as set forth SEQ ID NO: 52. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 42, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 52. In some cases, the antibody provided herein comprises a heavy chain variable region comprising an amino acid sequence having less than or equal to 10 total substitutions, deletions, and/or insertions in the amino acid sequence as set forth SEQ ID NO: 42, and a light chain variable region comprising an amino acid sequence having less than or equal to 10 total substitutions, deletions, and/or insertions in the amino acid sequence as set forth SEQ ID NO: 52. In some cases, the antibody provided herein comprises a heavy chain variable region comprising an amino acid sequence having less than or equal to 7 total substitutions, deletions, and/or insertions in the amino acid sequence as set forth SEQ ID NO: 42, and a light chain variable region comprising an amino acid sequence having less than or equal to 7 total substitutions, deletions, and/or insertions in the amino acid sequence as set forth SEQ ID NO: 52. In some cases, the antibody provided herein comprises a heavy chain variable region comprising an amino acid sequence having less than or equal to 5 total substitutions, deletions, and/or insertions in the amino acid sequence as set forth SEQ ID NO: 42, and a light chain variable region comprising an amino acid sequence having less than or equal to 5 total substitutions, deletions, and/or insertions in the amino acid sequence as set forth SEQ ID NO: 52. In some cases, the antibody provided herein comprises a heavy chain variable region comprising an amino acid sequence having less than or equal to 3 total substitutions, deletions, and/or insertions in the amino acid sequence as set forth SEQ ID NO: 42, and a light chain variable region comprising an amino acid sequence having less than or equal to 3 total substitutions, deletions, and/or insertions in the amino acid sequence as set forth SEQ ID NO: 52. In some cases, the antibody provided herein comprises a heavy chain variable region comprising an amino acid sequence having less than or equal to 2 total substitutions, deletions, and/or insertions in the amino acid sequence as set forth SEQ ID NO: 42, and a light chain variable region comprising an amino acid sequence having less than or equal to 2 total substitutions, deletions, and/or insertions in the amino acid sequence as set forth SEQ ID NO: 52. In some cases, the antibody provided herein comprises a heavy chain variable region comprising an amino acid sequence having less than or equal to 1 total substitutions, deletions, and/or insertions in the amino acid sequence as set forth SEQ ID NO: 42, and a light chain variable region comprising an amino acid sequence having less than or equal to 1 total substitutions, deletions, and/or insertions in the amino acid sequence as set forth SEQ ID NO: 52.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 42, and a light chain variable region comprising an amino acid sequence having at least 90%, 95%, 96%, 97%, 98%, 99%, or 100% sequence identity to the amino acid sequence as set forth SEQ ID NO: 65. In some cases, the antibody provided herein comprises a heavy chain variable region comprising an amino acid sequence having at least 95% sequence identity to the amino acid sequence as set forth SEQ ID NO: 42, and a light chain variable region comprising an amino acid sequence having at least 95% sequence identity to the amino acid sequence as set forth SEQ ID NO: 65. In some cases, the antibody provided herein comprises a heavy chain variable region comprising an amino acid sequence having at least 97% sequence identity to the amino acid sequence as set forth SEQ ID NO: 42, and a light chain variable region comprising an amino acid sequence having at least 97% sequence identity to the amino acid sequence as set forth SEQ ID NO: 65. In some cases, the antibody provided herein comprises a heavy chain variable region comprising an amino acid sequence having at least 99% sequence identity to the amino acid sequence as set forth SEQ ID NO: 42, and a light chain variable region comprising an amino acid sequence having at least 99% sequence identity to the amino acid sequence as set forth SEQ ID NO: 65. In some cases, the antibody provided herein comprises a heavy chain variable region comprising an amino acid sequence having 100% sequence identity to the amino acid sequence as set forth SEQ ID NO: 42, and a light chain variable region comprising an amino acid sequence having 100% sequence identity to the amino acid sequence as set forth SEQ ID NO: 65. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 42, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 65. In some cases, the antibody provided herein comprises a heavy chain variable region comprising an amino acid sequence having less than or equal to 10 total substitutions, deletions, and/or insertions in the amino acid sequence as set forth SEQ ID NO: 42, and a light chain variable region comprising an amino acid sequence having less than or equal to 10 total substitutions, deletions, and/or insertions in the amino acid sequence as set forth SEQ ID NO: 65. In some cases, the antibody provided herein comprises a heavy chain variable region comprising an amino acid sequence having less than or equal to 7 total substitutions, deletions, and/or insertions in the amino acid sequence as set forth SEQ ID NO: 42, and a light chain variable region comprising an amino acid sequence having less than or equal to 7 total substitutions, deletions, and/or insertions in the amino acid sequence as set forth SEQ ID NO: 65. In some cases, the antibody provided herein comprises a heavy chain variable region comprising an amino acid sequence having less than or equal to 5 total substitutions, deletions, and/or insertions in the amino acid sequence as set forth SEQ ID NO: 42, and a light chain variable region comprising an amino acid sequence having less than or equal to 5 total substitutions, deletions, and/or insertions in the amino acid sequence as set forth SEQ ID NO: 65. In some cases, the antibody provided herein comprises a heavy chain variable region comprising an amino acid sequence having less than or equal to 3 total substitutions, deletions, and/or insertions in the amino acid sequence as set forth SEQ ID NO: 42, and a light chain variable region comprising an amino acid sequence having less than or equal to 3 total substitutions, deletions, and/or insertions in the amino acid sequence as set forth SEQ ID NO: 65. In some cases, the antibody provided herein comprises a heavy chain variable region comprising an amino acid sequence having less than or equal to 2 total substitutions, deletions, and/or insertions in the amino acid sequence as set forth SEQ ID NO: 42, and a light chain variable region comprising an amino acid sequence having less than or equal to 2 total substitutions, deletions, and/or insertions in the amino acid sequence as set forth SEQ ID NO: 65. In some cases, the antibody provided herein comprises a heavy chain variable region comprising an amino acid sequence having less than or equal to 1 total substitutions, deletions, and/or insertions in the amino acid sequence as set forth SEQ ID NO: 42, and a light chain variable region comprising an amino acid sequence having less than or equal to 1 total substitutions, deletions, and/or insertions in the amino acid sequence as set forth SEQ ID NO: 65.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 42, and a light chain variable region comprising an amino acid sequence having at least 90%, 95%, 96%, 97%, 98%, 99%, or 100% sequence identity to the amino acid sequence as set forth SEQ ID NO: 66. In some cases, the antibody provided herein comprises a heavy chain variable region comprising an amino acid sequence having at least 95% sequence identity to the amino acid sequence as set forth SEQ ID NO: 42, and a light chain variable region comprising an amino acid sequence having at least 95% sequence identity to the amino acid sequence as set forth SEQ ID NO: 66. In some cases, the antibody provided herein comprises a heavy chain variable region comprising an amino acid sequence having at least 97% sequence identity to the amino acid sequence as set forth SEQ ID NO: 42, and a light chain variable region comprising an amino acid sequence having at least 97% sequence identity to the amino acid sequence as set forth SEQ ID NO: 66. In some cases, the antibody provided herein comprises a heavy chain variable region comprising an amino acid sequence having at least 99% sequence identity to the amino acid sequence as set forth SEQ ID NO: 42, and a light chain variable region comprising an amino acid sequence having at least 99% sequence identity to the amino acid sequence as set forth SEQ ID NO: 66. In some cases, the antibody provided herein comprises a heavy chain variable region comprising an amino acid sequence having 100% sequence identity to the amino acid sequence as set forth SEQ ID NO: 42, and a light chain variable region comprising an amino acid sequence having 100% sequence identity to the amino acid sequence as set forth SEQ ID NO: 66. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 42, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 66. In some cases, the antibody provided herein comprises a heavy chain variable region comprising an amino acid sequence having less than or equal to 10 total substitutions, deletions, and/or insertions in the amino acid sequence as set forth SEQ ID NO: 42, and a light chain variable region comprising an amino acid sequence having less than or equal to 10 total substitutions, deletions, and/or insertions in the amino acid sequence as set forth SEQ ID NO: 66. In some cases, the antibody provided herein comprises a heavy chain variable region comprising an amino acid sequence having less than or equal to 7 total substitutions, deletions, and/or insertions in the amino acid sequence as set forth SEQ ID NO: 42, and a light chain variable region comprising an amino acid sequence having less than or equal to 7 total substitutions, deletions, and/or insertions in the amino acid sequence as set forth SEQ ID NO: 66. In some cases, the antibody provided herein comprises a heavy chain variable region comprising an amino acid sequence having less than or equal to 5 total substitutions, deletions, and/or insertions in the amino acid sequence as set forth SEQ ID NO: 42, and a light chain variable region comprising an amino acid sequence having less than or equal to 5 total substitutions, deletions, and/or insertions in the amino acid sequence as set forth SEQ ID NO: 66. In some cases, the antibody provided herein comprises a heavy chain variable region comprising an amino acid sequence having less than or equal to 3 total substitutions, deletions, and/or insertions in the amino acid sequence as set forth SEQ ID NO: 42, and a light chain variable region comprising an amino acid sequence having less than or equal to 3 total substitutions, deletions, and/or insertions in the amino acid sequence as set forth SEQ ID NO: 66. In some cases, the antibody provided herein comprises a heavy chain variable region comprising an amino acid sequence having less than or equal to 2 total substitutions, deletions, and/or insertions in the amino acid sequence as set forth SEQ ID NO: 42, and a light chain variable region comprising an amino acid sequence having less than or equal to 2 total substitutions, deletions, and/or insertions in the amino acid sequence as set forth SEQ ID NO: 66. In some cases, the antibody provided herein comprises a heavy chain variable region comprising an amino acid sequence having less than or equal to 1 total substitutions, deletions, and/or insertions in the amino acid sequence as set forth SEQ ID NO: 42, and a light chain variable region comprising an amino acid sequence having less than or equal to 1 total substitutions, deletions, and/or insertions in the amino acid sequence as set forth SEQ ID NO: 66.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 43, and a light chain variable region comprising an amino acid sequence having at least 90%, 95%, 96%, 97%, 98%, 99%, or 100% sequence identity to the amino acid sequence as set forth SEQ ID NO: 2. In some cases, the antibody provided herein comprises a heavy chain variable region comprising an amino acid sequence having at least 95% sequence identity to the amino acid sequence as set forth SEQ ID NO: 43, and a light chain variable region comprising an amino acid sequence having at least 95% sequence identity to the amino acid sequence as set forth SEQ ID NO: 2. In some cases, the antibody provided herein comprises a heavy chain variable region comprising an amino acid sequence having at least 97% sequence identity to the amino acid sequence as set forth SEQ ID NO: 43, and a light chain variable region comprising an amino acid sequence having at least 97% sequence identity to the amino acid sequence as set forth SEQ ID NO: 2. In some cases, the antibody provided herein comprises a heavy chain variable region comprising an amino acid sequence having at least 99% sequence identity to the amino acid sequence as set forth SEQ ID NO: 43, and a light chain variable region comprising an amino acid sequence having at least 99% sequence identity to the amino acid sequence as set forth SEQ ID NO: 2. In some cases, the antibody provided herein comprises a heavy chain variable region comprising an amino acid sequence having 100% sequence identity to the amino acid sequence as set forth SEQ ID NO: 43, and a light chain variable region comprising an amino acid sequence having 100% sequence identity to the amino acid sequence as set forth SEQ ID NO: 2. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 43, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 2. In some cases, the antibody provided herein comprises a heavy chain variable region comprising an amino acid sequence having less than or equal to 10 total substitutions, deletions, and/or insertions in the amino acid sequence as set forth SEQ ID NO: 43, and a light chain variable region comprising an amino acid sequence having less than or equal to 10 total substitutions, deletions, and/or insertions in the amino acid sequence as set forth SEQ ID NO: 2. In some cases, the antibody provided herein comprises a heavy chain variable region comprising an amino acid sequence having less than or equal to 7 total substitutions, deletions, and/or insertions in the amino acid sequence as set forth SEQ ID NO: 43, and a light chain variable region comprising an amino acid sequence having less than or equal to 7 total substitutions, deletions, and/or insertions in the amino acid sequence as set forth SEQ ID NO: 2. In some cases, the antibody provided herein comprises a heavy chain variable region comprising an amino acid sequence having less than or equal to 5 total substitutions, deletions, and/or insertions in the amino acid sequence as set forth SEQ ID NO: 43, and a light chain variable region comprising an amino acid sequence having less than or equal to 5 total substitutions, deletions, and/or insertions in the amino acid sequence as set forth SEQ ID NO: 2. In some cases, the antibody provided herein comprises a heavy chain variable region comprising an amino acid sequence having less than or equal to 3 total substitutions, deletions, and/or insertions in the amino acid sequence as set forth SEQ ID NO: 43, and a light chain variable region comprising an amino acid sequence having less than or equal to 3 total substitutions, deletions, and/or insertions in the amino acid sequence as set forth SEQ ID NO: 2. In some cases, the antibody provided herein comprises a heavy chain variable region comprising an amino acid sequence having less than or equal to 2 total substitutions, deletions, and/or insertions in the amino acid sequence as set forth SEQ ID NO: 43, and a light chain variable region comprising an amino acid sequence having less than or equal to 2 total substitutions, deletions, and/or insertions in the amino acid sequence as set forth SEQ ID NO: 2. In some cases, the antibody provided herein comprises a heavy chain variable region comprising an amino acid sequence having less than or equal to 1 total substitutions, deletions, and/or insertions in the amino acid sequence as set forth SEQ ID NO: 43, and a light chain variable region comprising an amino acid sequence having less than or equal to 1 total substitutions, deletions, and/or insertions in the amino acid sequence as set forth SEQ ID NO: 2.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 43, and a light chain variable region comprising an amino acid sequence having at least 90%, 95%, 96%, 97%, 98%, 99%, or 100% sequence identity to the amino acid sequence as set forth SEQ ID NO: 48. In some cases, the antibody provided herein comprises a heavy chain variable region comprising an amino acid sequence having at least 95% sequence identity to the amino acid sequence as set forth SEQ ID NO: 43, and a light chain variable region comprising an amino acid sequence having at least 95% sequence identity to the amino acid sequence as set forth SEQ ID NO: 48. In some cases, the antibody provided herein comprises a heavy chain variable region comprising an amino acid sequence having at least 97% sequence identity to the amino acid sequence as set forth SEQ ID NO: 43, and a light chain variable region comprising an amino acid sequence having at least 97% sequence identity to the amino acid sequence as set forth SEQ ID NO: 48. In some cases, the antibody provided herein comprises a heavy chain variable region comprising an amino acid sequence having at least 99% sequence identity to the amino acid sequence as set forth SEQ ID NO: 43, and a light chain variable region comprising an amino acid sequence having at least 99% sequence identity to the amino acid sequence as set forth SEQ ID NO: 48. In some cases, the antibody provided herein comprises a heavy chain variable region comprising an amino acid sequence having 100% sequence identity to the amino acid sequence as set forth SEQ ID NO: 43, and a light chain variable region comprising an amino acid sequence having 100% sequence identity to the amino acid sequence as set forth SEQ ID NO: 48. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 43, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 48. In some cases, the antibody provided herein comprises a heavy chain variable region comprising an amino acid sequence having less than or equal to 10 total substitutions, deletions, and/or insertions in the amino acid sequence as set forth SEQ ID NO: 43, and a light chain variable region comprising an amino acid sequence having less than or equal to 10 total substitutions, deletions, and/or insertions in the amino acid sequence as set forth SEQ ID NO: 48. In some cases, the antibody provided herein comprises a heavy chain variable region comprising an amino acid sequence having less than or equal to 7 total substitutions, deletions, and/or insertions in the amino acid sequence as set forth SEQ ID NO: 43, and a light chain variable region comprising an amino acid sequence having less than or equal to 7 total substitutions, deletions, and/or insertions in the amino acid sequence as set forth SEQ ID NO: 48. In some cases, the antibody provided herein comprises a heavy chain variable region comprising an amino acid sequence having less than or equal to 5 total substitutions, deletions, and/or insertions in the amino acid sequence as set forth SEQ ID NO: 43, and a light chain variable region comprising an amino acid sequence having less than or equal to 5 total substitutions, deletions, and/or insertions in the amino acid sequence as set forth SEQ ID NO: 48. In some cases, the antibody provided herein comprises a heavy chain variable region comprising an amino acid sequence having less than or equal to 3 total substitutions, deletions, and/or insertions in the amino acid sequence as set forth SEQ ID NO: 43, and a light chain variable region comprising an amino acid sequence having less than or equal to 3 total substitutions, deletions, and/or insertions in the amino acid sequence as set forth SEQ ID NO: 48. In some cases, the antibody provided herein comprises a heavy chain variable region comprising an amino acid sequence having less than or equal to 2 total substitutions, deletions, and/or insertions in the amino acid sequence as set forth SEQ ID NO: 43, and a light chain variable region comprising an amino acid sequence having less than or equal to 2 total substitutions, deletions, and/or insertions in the amino acid sequence as set forth SEQ ID NO: 48. In some cases, the antibody provided herein comprises a heavy chain variable region comprising an amino acid sequence having less than or equal to 1 total substitutions, deletions, and/or insertions in the amino acid sequence as set forth SEQ ID NO: 43, and a light chain variable region comprising an amino acid sequence having less than or equal to 1 total substitutions, deletions, and/or insertions in the amino acid sequence as set forth SEQ ID NO: 48

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 43, and a light chain variable region comprising an amino acid sequence having at least 90%, 95%, 96%, 97%, 98%, 99%, or 100% sequence identity to the amino acid sequence as set forth SEQ ID NO: 49. In some cases, the antibody provided herein comprises a heavy chain variable region comprising an amino acid sequence having at least 95% sequence identity to the amino acid sequence as set forth SEQ ID NO: 43, and a light chain variable region comprising an amino acid sequence having at least 95% sequence identity to the amino acid sequence as set forth SEQ ID NO: 49. In some cases, the antibody provided herein comprises a heavy chain variable region comprising an amino acid sequence having at least 97% sequence identity to the amino acid sequence as set forth SEQ ID NO: 43, and a light chain variable region comprising an amino acid sequence having at least 97% sequence identity to the amino acid sequence as set forth SEQ ID NO: 49. In some cases, the antibody provided herein comprises a heavy chain variable region comprising an amino acid sequence having at least 99% sequence identity to the amino acid sequence as set forth SEQ ID NO: 43, and a light chain variable region comprising an amino acid sequence having at least 99% sequence identity to the amino acid sequence as set forth SEQ ID NO: 49. In some cases, the antibody provided herein comprises a heavy chain variable region comprising an amino acid sequence having 100% sequence identity to the amino acid sequence as set forth SEQ ID NO: 43, and a light chain variable region comprising an amino acid sequence having 100% sequence identity to the amino acid sequence as set forth SEQ ID NO: 49. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 43, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 49. In some cases, the antibody provided herein comprises a heavy chain variable region comprising an amino acid sequence having less than or equal to 10 total substitutions, deletions, and/or insertions in the amino acid sequence as set forth SEQ ID NO: 43, and a light chain variable region comprising an amino acid sequence having less than or equal to 10 total substitutions, deletions, and/or insertions in the amino acid sequence as set forth SEQ ID NO: 49. In some cases, the antibody provided herein comprises a heavy chain variable region comprising an amino acid sequence having less than or equal to 7 total substitutions, deletions, and/or insertions in the amino acid sequence as set forth SEQ ID NO: 43, and a light chain variable region comprising an amino acid sequence having less than or equal to 7 total substitutions, deletions, and/or insertions in the amino acid sequence as set forth SEQ ID NO: 49. In some cases, the antibody provided herein comprises a heavy chain variable region comprising an amino acid sequence having less than or equal to 5 total substitutions, deletions, and/or insertions in the amino acid sequence as set forth SEQ ID NO: 43, and a light chain variable region comprising an amino acid sequence having less than or equal to 5 total substitutions, deletions, and/or insertions in the amino acid sequence as set forth SEQ ID NO: 49. In some cases, the antibody provided herein comprises a heavy chain variable region comprising an amino acid sequence having less than or equal to 3 total substitutions, deletions, and/or insertions in the amino acid sequence as set forth SEQ ID NO: 43, and a light chain variable region comprising an amino acid sequence having less than or equal to 3 total substitutions, deletions, and/or insertions in the amino acid sequence as set forth SEQ ID NO: 49. In some cases, the antibody provided herein comprises a heavy chain variable region comprising an amino acid sequence having less than or equal to 2 total substitutions, deletions, and/or insertions in the amino acid sequence as set forth SEQ ID NO: 43, and a light chain variable region comprising an amino acid sequence having less than or equal to 2 total substitutions, deletions, and/or insertions in the amino acid sequence as set forth SEQ ID NO: 49. In some cases, the antibody provided herein comprises a heavy chain variable region comprising an amino acid sequence having less than or equal to 1 total substitutions, deletions, and/or insertions in the amino acid sequence as set forth SEQ ID NO: 43, and a light chain variable region comprising an amino acid sequence having less than or equal to 1 total substitutions, deletions, and/or insertions in the amino acid sequence as set forth SEQ ID NO: 49.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 43, and a light chain variable region comprising an amino acid sequence having at least 90%, 95%, 96%, 97%, 98%, 99%, or 100% sequence identity to the amino acid sequence as set forth SEQ ID NO: 50. In some cases, the antibody provided herein comprises a heavy chain variable region comprising an amino acid sequence having at least 95% sequence identity to the amino acid sequence as set forth SEQ ID NO: 43, and a light chain variable region comprising an amino acid sequence having at least 95% sequence identity to the amino acid sequence as set forth SEQ ID NO: 50. In some cases, the antibody provided herein comprises a heavy chain variable region comprising an amino acid sequence having at least 97% sequence identity to the amino acid sequence as set forth SEQ ID NO: 43, and a light chain variable region comprising an amino acid sequence having at least 97% sequence identity to the amino acid sequence as set forth SEQ ID NO: 50. In some cases, the antibody provided herein comprises a heavy chain variable region comprising an amino acid sequence having at least 99% sequence identity to the amino acid sequence as set forth SEQ ID NO: 43, and a light chain variable region comprising an amino acid sequence having at least 99% sequence identity to the amino acid sequence as set forth SEQ ID NO: 50. In some cases, the antibody provided herein comprises a heavy chain variable region comprising an amino acid sequence having 100% sequence identity to the amino acid sequence as set forth SEQ ID NO: 43, and a light chain variable region comprising an amino acid sequence having 100% sequence identity to the amino acid sequence as set forth SEQ ID NO: 50. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 43, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 50. In some cases, the antibody provided herein comprises a heavy chain variable region comprising an amino acid sequence having less than or equal to 10 total substitutions, deletions, and/or insertions in the amino acid sequence as set forth SEQ ID NO: 43, and a light chain variable region comprising an amino acid sequence having less than or equal to 10 total substitutions, deletions, and/or insertions in the amino acid sequence as set forth SEQ ID NO: 50. In some cases, the antibody provided herein comprises a heavy chain variable region comprising an amino acid sequence having less than or equal to 7 total substitutions, deletions, and/or insertions in the amino acid sequence as set forth SEQ ID NO: 43, and a light chain variable region comprising an amino acid sequence having less than or equal to 7 total substitutions, deletions, and/or insertions in the amino acid sequence as set forth SEQ ID NO: 50. In some cases, the antibody provided herein comprises a heavy chain variable region comprising an amino acid sequence having less than or equal to 5 total substitutions, deletions, and/or insertions in the amino acid sequence as set forth SEQ ID NO: 43, and a light chain variable region comprising an amino acid sequence having less than or equal to 5 total substitutions, deletions, and/or insertions in the amino acid sequence as set forth SEQ ID NO: 50. In some cases, the antibody provided herein comprises a heavy chain variable region comprising an amino acid sequence having less than or equal to 3 total substitutions, deletions, and/or insertions in the amino acid sequence as set forth SEQ ID NO: 43, and a light chain variable region comprising an amino acid sequence having less than or equal to 3 total substitutions, deletions, and/or insertions in the amino acid sequence as set forth SEQ ID NO: 50. In some cases, the antibody provided herein comprises a heavy chain variable region comprising an amino acid sequence having less than or equal to 2 total substitutions, deletions, and/or insertions in the amino acid sequence as set forth SEQ ID NO: 43, and a light chain variable region comprising an amino acid sequence having less than or equal to 2 total substitutions, deletions, and/or insertions in the amino acid sequence as set forth SEQ ID NO: 50. In some cases, the antibody provided herein comprises a heavy chain variable region comprising an amino acid sequence having less than or equal to 1 total substitutions, deletions, and/or insertions in the amino acid sequence as set forth SEQ ID NO: 43, and a light chain variable region comprising an amino acid sequence having less than or equal to 1 total substitutions, deletions, and/or insertions in the amino acid sequence as set forth SEQ ID NO: 50.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 43, and a light chain variable region comprising an amino acid sequence having at least 90%, 95%, 96%, 97%, 98%, 99%, or 100% sequence identity to the amino acid sequence as set forth SEQ ID NO: 51. In some cases, the antibody provided herein comprises a heavy chain variable region comprising an amino acid sequence having at least 95% sequence identity to the amino acid sequence as set forth SEQ ID NO: 43, and a light chain variable region comprising an amino acid sequence having at least 95% sequence identity to the amino acid sequence as set forth SEQ ID NO: 51. In some cases, the antibody provided herein comprises a heavy chain variable region comprising an amino acid sequence having at least 97% sequence identity to the amino acid sequence as set forth SEQ ID NO: 43, and a light chain variable region comprising an amino acid sequence having at least 97% sequence identity to the amino acid sequence as set forth SEQ ID NO: 51. In some cases, the antibody provided herein comprises a heavy chain variable region comprising an amino acid sequence having at least 99% sequence identity to the amino acid sequence as set forth SEQ ID NO: 43, and a light chain variable region comprising an amino acid sequence having at least 99% sequence identity to the amino acid sequence as set forth SEQ ID NO: 51. In some cases, the antibody provided herein comprises a heavy chain variable region comprising an amino acid sequence having 100% sequence identity to the amino acid sequence as set forth SEQ ID NO: 43, and a light chain variable region comprising an amino acid sequence having 100% sequence identity to the amino acid sequence as set forth SEQ ID NO: 51. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 43, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 51. In some cases, the antibody provided herein comprises a heavy chain variable region comprising an amino acid sequence having less than or equal to 10 total substitutions, deletions, and/or insertions in the amino acid sequence as set forth SEQ ID NO: 43, and a light chain variable region comprising an amino acid sequence having less than or equal to 10 total substitutions, deletions, and/or insertions in the amino acid sequence as set forth SEQ ID NO: 51. In some cases, the antibody provided herein comprises a heavy chain variable region comprising an amino acid sequence having less than or equal to 7 total substitutions, deletions, and/or insertions in the amino acid sequence as set forth SEQ ID NO: 43, and a light chain variable region comprising an amino acid sequence having less than or equal to 7 total substitutions, deletions, and/or insertions in the amino acid sequence as set forth SEQ ID NO: 51. In some cases, the antibody provided herein comprises a heavy chain variable region comprising an amino acid sequence having less than or equal to 5 total substitutions, deletions, and/or insertions in the amino acid sequence as set forth SEQ ID NO: 43, and a light chain variable region comprising an amino acid sequence having less than or equal to 5 total substitutions, deletions, and/or insertions in the amino acid sequence as set forth SEQ ID NO: 51. In some cases, the antibody provided herein comprises a heavy chain variable region comprising an amino acid sequence having less than or equal to 3 total substitutions, deletions, and/or insertions in the amino acid sequence as set forth SEQ ID NO: 43, and a light chain variable region comprising an amino acid sequence having less than or equal to 3 total substitutions, deletions, and/or insertions in the amino acid sequence as set forth SEQ ID NO: 51. In some cases, the antibody provided herein comprises a heavy chain variable region comprising an amino acid sequence having less than or equal to 2 total substitutions, deletions, and/or insertions in the amino acid sequence as set forth SEQ ID NO: 43, and a light chain variable region comprising an amino acid sequence having less than or equal to 2 total substitutions, deletions, and/or insertions in the amino acid sequence as set forth SEQ ID NO: 51. In some cases, the antibody provided herein comprises a heavy chain variable region comprising an amino acid sequence having less than or equal to 1 total substitutions, deletions, and/or insertions in the amino acid sequence as set forth SEQ ID NO: 43, and a light chain variable region comprising an amino acid sequence having less than or equal to 1 total substitutions, deletions, and/or insertions in the amino acid sequence as set forth SEQ ID NO: 51

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 43, and a light chain variable region comprising an amino acid sequence having at least 90%, 95%, 96%, 97%, 98%, 99%, or 100% sequence identity to the amino acid sequence as set forth SEQ ID NO: 52. In some cases, the antibody provided herein comprises a heavy chain variable region comprising an amino acid sequence having at least 95% sequence identity to the amino acid sequence as set forth SEQ ID NO: 43, and a light chain variable region comprising an amino acid sequence having at least 95% sequence identity to the amino acid sequence as set forth SEQ ID NO: 52. In some cases, the antibody provided herein comprises a heavy chain variable region comprising an amino acid sequence having at least 97% sequence identity to the amino acid sequence as set forth SEQ ID NO: 43, and a light chain variable region comprising an amino acid sequence having at least 97% sequence identity to the amino acid sequence as set forth SEQ ID NO: 52. In some cases, the antibody provided herein comprises a heavy chain variable region comprising an amino acid sequence having at least 99% sequence identity to the amino acid sequence as set forth SEQ ID NO: 43, and a light chain variable region comprising an amino acid sequence having at least 99% sequence identity to the amino acid sequence as set forth SEQ ID NO: 52. In some cases, the antibody provided herein comprises a heavy chain variable region comprising an amino acid sequence having 100% sequence identity to the amino acid sequence as set forth SEQ ID NO: 43, and a light chain variable region comprising an amino acid sequence having 100% sequence identity to the amino acid sequence as set forth SEQ ID NO: 52. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 43, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 52. In some cases, the antibody provided herein comprises a heavy chain variable region comprising an amino acid sequence having less than or equal to 10 total substitutions, deletions, and/or insertions in the amino acid sequence as set forth SEQ ID NO: 43, and a light chain variable region comprising an amino acid sequence having less than or equal to 10 total substitutions, deletions, and/or insertions in the amino acid sequence as set forth SEQ ID NO: 52. In some cases, the antibody provided herein comprises a heavy chain variable region comprising an amino acid sequence having less than or equal to 7 total substitutions, deletions, and/or insertions in the amino acid sequence as set forth SEQ ID NO: 43, and a light chain variable region comprising an amino acid sequence having less than or equal to 7 total substitutions, deletions, and/or insertions in the amino acid sequence as set forth SEQ ID NO: 52. In some cases, the antibody provided herein comprises a heavy chain variable region comprising an amino acid sequence having less than or equal to 5 total substitutions, deletions, and/or insertions in the amino acid sequence as set forth SEQ ID NO: 43, and a light chain variable region comprising an amino acid sequence having less than or equal to 5 total substitutions, deletions, and/or insertions in the amino acid sequence as set forth SEQ ID NO: 52. In some cases, the antibody provided herein comprises a heavy chain variable region comprising an amino acid sequence having less than or equal to 3 total substitutions, deletions, and/or insertions in the amino acid sequence as set forth SEQ ID NO: 43, and a light chain variable region comprising an amino acid sequence having less than or equal to 3 total substitutions, deletions, and/or insertions in the amino acid sequence as set forth SEQ ID NO: 52. In some cases, the antibody provided herein comprises a heavy chain variable region comprising an amino acid sequence having less than or equal to 2 total substitutions, deletions, and/or insertions in the amino acid sequence as set forth SEQ ID NO: 43, and a light chain variable region comprising an amino acid sequence having less than or equal to 2 total substitutions, deletions, and/or insertions in the amino acid sequence as set forth SEQ ID NO: 52. In some cases, the antibody provided herein comprises a heavy chain variable region comprising an amino acid sequence having less than or equal to 1 total substitutions, deletions, and/or insertions in the amino acid sequence as set forth SEQ ID NO: 43, and a light chain variable region comprising an amino acid sequence having less than or equal to 1 total substitutions, deletions, and/or insertions in the amino acid sequence as set forth SEQ ID NO: 52.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 43, and a light chain variable region comprising an amino acid sequence having at least 90%, 95%, 96%, 97%, 98%, 99%, or 100% sequence identity to the amino acid sequence as set forth SEQ ID NO: 65. In some cases, the antibody provided herein comprises a heavy chain variable region comprising an amino acid sequence having at least 95% sequence identity to the amino acid sequence as set forth SEQ ID NO: 43, and a light chain variable region comprising an amino acid sequence having at least 95% sequence identity to the amino acid sequence as set forth SEQ ID NO: 65. In some cases, the antibody provided herein comprises a heavy chain variable region comprising an amino acid sequence having at least 97% sequence identity to the amino acid sequence as set forth SEQ ID NO: 43, and a light chain variable region comprising an amino acid sequence having at least 97% sequence identity to the amino acid sequence as set forth SEQ ID NO: 65. In some cases, the antibody provided herein comprises a heavy chain variable region comprising an amino acid sequence having at least 99% sequence identity to the amino acid sequence as set forth SEQ ID NO: 43, and a light chain variable region comprising an amino acid sequence having at least 99% sequence identity to the amino acid sequence as set forth SEQ ID NO: 65. In some cases, the antibody provided herein comprises a heavy chain variable region comprising an amino acid sequence having 100% sequence identity to the amino acid sequence as set forth SEQ ID NO: 43, and a light chain variable region comprising an amino acid sequence having 100% sequence identity to the amino acid sequence as set forth SEQ ID NO: 65. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 43, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 65. In some cases, the antibody provided herein comprises a heavy chain variable region comprising an amino acid sequence having less than or equal to 10 total substitutions, deletions, and/or insertions in the amino acid sequence as set forth SEQ ID NO: 43, and a light chain variable region comprising an amino acid sequence having less than or equal to 10 total substitutions, deletions, and/or insertions in the amino acid sequence as set forth SEQ ID NO: 65. In some cases, the antibody provided herein comprises a heavy chain variable region comprising an amino acid sequence having less than or equal to 7 total substitutions, deletions, and/or insertions in the amino acid sequence as set forth SEQ ID NO: 43, and a light chain variable region comprising an amino acid sequence having less than or equal to 7 total substitutions, deletions, and/or insertions in the amino acid sequence as set forth SEQ ID NO: 65. In some cases, the antibody provided herein comprises a heavy chain variable region comprising an amino acid sequence having less than or equal to 5 total substitutions, deletions, and/or insertions in the amino acid sequence as set forth SEQ ID NO: 43, and a light chain variable region comprising an amino acid sequence having less than or equal to 5 total substitutions, deletions, and/or insertions in the amino acid sequence as set forth SEQ ID NO: 65. In some cases, the antibody provided herein comprises a heavy chain variable region comprising an amino acid sequence having less than or equal to 3 total substitutions, deletions, and/or insertions in the amino acid sequence as set forth SEQ ID NO: 43, and a light chain variable region comprising an amino acid sequence having less than or equal to 3 total substitutions, deletions, and/or insertions in the amino acid sequence as set forth SEQ ID NO: 65. In some cases, the antibody provided herein comprises a heavy chain variable region comprising an amino acid sequence having less than or equal to 2 total substitutions, deletions, and/or insertions in the amino acid sequence as set forth SEQ ID NO: 43, and a light chain variable region comprising an amino acid sequence having less than or equal to 2 total substitutions, deletions, and/or insertions in the amino acid sequence as set forth SEQ ID NO: 65. In some cases, the antibody provided herein comprises a heavy chain variable region comprising an amino acid sequence having less than or equal to 1 total substitutions, deletions, and/or insertions in the amino acid sequence as set forth SEQ ID NO: 43, and a light chain variable region comprising an amino acid sequence having less than or equal to 1 total substitutions, deletions, and/or insertions in the amino acid sequence as set forth SEQ ID NO: 65.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 43, and a light chain variable region comprising an amino acid sequence having at least 90%, 95%, 96%, 97%, 98%, 99%, or 100% sequence identity to the amino acid sequence as set forth SEQ ID NO: 66. In some cases, the antibody provided herein comprises a heavy chain variable region comprising an amino acid sequence having at least 95% sequence identity to the amino acid sequence as set forth SEQ ID NO: 43, and a light chain variable region comprising an amino acid sequence having at least 95% sequence identity to the amino acid sequence as set forth SEQ ID NO: 66. In some cases, the antibody provided herein comprises a heavy chain variable region comprising an amino acid sequence having at least 97% sequence identity to the amino acid sequence as set forth SEQ ID NO: 43, and a light chain variable region comprising an amino acid sequence having at least 97% sequence identity to the amino acid sequence as set forth SEQ ID NO: 66. In some cases, the antibody provided herein comprises a heavy chain variable region comprising an amino acid sequence having at least 99% sequence identity to the amino acid sequence as set forth SEQ ID NO: 43, and a light chain variable region comprising an amino acid sequence having at least 99% sequence identity to the amino acid sequence as set forth SEQ ID NO: 66. In some cases, the antibody provided herein comprises a heavy chain variable region comprising an amino acid sequence having 100% sequence identity to the amino acid sequence as set forth SEQ ID NO: 43, and a light chain variable region comprising an amino acid sequence having 100% sequence identity to the amino acid sequence as set forth SEQ ID NO: 66. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 43, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 66. In some cases, the antibody provided herein comprises a heavy chain variable region comprising an amino acid sequence having less than or equal to 10 total substitutions, deletions, and/or insertions in the amino acid sequence as set forth SEQ ID NO: 43, and a light chain variable region comprising an amino acid sequence having less than or equal to 10 total substitutions, deletions, and/or insertions in the amino acid sequence as set forth SEQ ID NO: 66. In some cases, the antibody provided herein comprises a heavy chain variable region comprising an amino acid sequence having less than or equal to 7 total substitutions, deletions, and/or insertions in the amino acid sequence as set forth SEQ ID NO: 43, and a light chain variable region comprising an amino acid sequence having less than or equal to 7 total substitutions, deletions, and/or insertions in the amino acid sequence as set forth SEQ ID NO: 66. In some cases, the antibody provided herein comprises a heavy chain variable region comprising an amino acid sequence having less than or equal to 5 total substitutions, deletions, and/or insertions in the amino acid sequence as set forth SEQ ID NO: 43, and a light chain variable region comprising an amino acid sequence having less than or equal to 5 total substitutions, deletions, and/or insertions in the amino acid sequence as set forth SEQ ID NO: 66. In some cases, the antibody provided herein comprises a heavy chain variable region comprising an amino acid sequence having less than or equal to 3 total substitutions, deletions, and/or insertions in the amino acid sequence as set forth SEQ ID NO: 43, and a light chain variable region comprising an amino acid sequence having less than or equal to 3 total substitutions, deletions, and/or insertions in the amino acid sequence as set forth SEQ ID NO: 66. In some cases, the antibody provided herein comprises a heavy chain variable region comprising an amino acid sequence having less than or equal to 2 total substitutions, deletions, and/or insertions in the amino acid sequence as set forth SEQ ID NO: 43, and a light chain variable region comprising an amino acid sequence having less than or equal to 2 total substitutions, deletions, and/or insertions in the amino acid sequence as set forth SEQ ID NO: 66. In some cases, the antibody provided herein comprises a heavy chain variable region comprising an amino acid sequence having less than or equal to 1 total substitutions, deletions, and/or insertions in the amino acid sequence as set forth SEQ ID NO: 43, and a light chain variable region comprising an amino acid sequence having less than or equal to 1 total substitutions, deletions, and/or insertions in the amino acid sequence as set forth SEQ ID NO: 66.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 44, and a light chain variable region comprising an amino acid sequence having at least 90%, 95%, 96%, 97%, 98%, 99%, or 100% sequence identity to the amino acid sequence as set forth SEQ ID NO: 2. In some cases, the antibody provided herein comprises a heavy chain variable region comprising an amino acid sequence having at least 95% sequence identity to the amino acid sequence as set forth SEQ ID NO: 44, and a light chain variable region comprising an amino acid sequence having at least 95% sequence identity to the amino acid sequence as set forth SEQ ID NO: 2. In some cases, the antibody provided herein comprises a heavy chain variable region comprising an amino acid sequence having at least 97% sequence identity to the amino acid sequence as set forth SEQ ID NO: 44, and a light chain variable region comprising an amino acid sequence having at least 97% sequence identity to the amino acid sequence as set forth SEQ ID NO: 2. In some cases, the antibody provided herein comprises a heavy chain variable region comprising an amino acid sequence having at least 99% sequence identity to the amino acid sequence as set forth SEQ ID NO: 44, and a light chain variable region comprising an amino acid sequence having at least 99% sequence identity to the amino acid sequence as set forth SEQ ID NO: 2. In some cases, the antibody provided herein comprises a heavy chain variable region comprising an amino acid sequence having 100% sequence identity to the amino acid sequence as set forth SEQ ID NO: 44, and a light chain variable region comprising an amino acid sequence having 100% sequence identity to the amino acid sequence as set forth SEQ ID NO: 2. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 44, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 2. In some cases, the antibody provided herein comprises a heavy chain variable region comprising an amino acid sequence having less than or equal to 10 total substitutions, deletions, and/or insertions in the amino acid sequence as set forth SEQ ID NO: 44, and a light chain variable region comprising an amino acid sequence having less than or equal to 10 total substitutions, deletions, and/or insertions in the amino acid sequence as set forth SEQ ID NO: 2. In some cases, the antibody provided herein comprises a heavy chain variable region comprising an amino acid sequence having less than or equal to 7 total substitutions, deletions, and/or insertions in the amino acid sequence as set forth SEQ ID NO: 44, and a light chain variable region comprising an amino acid sequence having less than or equal to 7 total substitutions, deletions, and/or insertions in the amino acid sequence as set forth SEQ ID NO: 2. In some cases, the antibody provided herein comprises a heavy chain variable region comprising an amino acid sequence having less than or equal to 5 total substitutions, deletions, and/or insertions in the amino acid sequence as set forth SEQ ID NO: 44, and a light chain variable region comprising an amino acid sequence having less than or equal to 5 total substitutions, deletions, and/or insertions in the amino acid sequence as set forth SEQ ID NO: 2. In some cases, the antibody provided herein comprises a heavy chain variable region comprising an amino acid sequence having less than or equal to 3 total substitutions, deletions, and/or insertions in the amino acid sequence as set forth SEQ ID NO: 44, and a light chain variable region comprising an amino acid sequence having less than or equal to 3 total substitutions, deletions, and/or insertions in the amino acid sequence as set forth SEQ ID NO: 2. In some cases, the antibody provided herein comprises a heavy chain variable region comprising an amino acid sequence having less than or equal to 2 total substitutions, deletions, and/or insertions in the amino acid sequence as set forth SEQ ID NO: 44, and a light chain variable region comprising an amino acid sequence having less than or equal to 2 total substitutions, deletions, and/or insertions in the amino acid sequence as set forth SEQ ID NO: 2. In some cases, the antibody provided herein comprises a heavy chain variable region comprising an amino acid sequence having less than or equal to 1 total substitutions, deletions, and/or insertions in the amino acid sequence as set forth SEQ ID NO: 44, and a light chain variable region comprising an amino acid sequence having less than or equal to 1 total substitutions, deletions, and/or insertions in the amino acid sequence as set forth SEQ ID NO: 2.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 44, and a light chain variable region comprising an amino acid sequence having at least 90%, 95%, 96%, 97%, 98%, 99%, or 100% sequence identity to the amino acid sequence as set forth SEQ ID NO: 48. In some cases, the antibody provided herein comprises a heavy chain variable region comprising an amino acid sequence having at least 95% sequence identity to the amino acid sequence as set forth SEQ ID NO: 44, and a light chain variable region comprising an amino acid sequence having at least 95% sequence identity to the amino acid sequence as set forth SEQ ID NO: 48. In some cases, the antibody provided herein comprises a heavy chain variable region comprising an amino acid sequence having at least 97% sequence identity to the amino acid sequence as set forth SEQ ID NO: 44, and a light chain variable region comprising an amino acid sequence having at least 97% sequence identity to the amino acid sequence as set forth SEQ ID NO: 48. In some cases, the antibody provided herein comprises a heavy chain variable region comprising an amino acid sequence having at least 99% sequence identity to the amino acid sequence as set forth SEQ ID NO: 44, and a light chain variable region comprising an amino acid sequence having at least 99% sequence identity to the amino acid sequence as set forth SEQ ID NO: 48. In some cases, the antibody provided herein comprises a heavy chain variable region comprising an amino acid sequence having 100% sequence identity to the amino acid sequence as set forth SEQ ID NO: 44, and a light chain variable region comprising an amino acid sequence having 100% sequence identity to the amino acid sequence as set forth SEQ ID NO: 48. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 44, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 48. In some cases, the antibody provided herein comprises a heavy chain variable region comprising an amino acid sequence having less than or equal to 10 total substitutions, deletions, and/or insertions in the amino acid sequence as set forth SEQ ID NO: 44, and a light chain variable region comprising an amino acid sequence having less than or equal to 10 total substitutions, deletions, and/or insertions in the amino acid sequence as set forth SEQ ID NO: 48. In some cases, the antibody provided herein comprises a heavy chain variable region comprising an amino acid sequence having less than or equal to 7 total substitutions, deletions, and/or insertions in the amino acid sequence as set forth SEQ ID NO: 44, and a light chain variable region comprising an amino acid sequence having less than or equal to 7 total substitutions, deletions, and/or insertions in the amino acid sequence as set forth SEQ ID NO: 48. In some cases, the antibody provided herein comprises a heavy chain variable region comprising an amino acid sequence having less than or equal to 5 total substitutions, deletions, and/or insertions in the amino acid sequence as set forth SEQ ID NO: 44, and a light chain variable region comprising an amino acid sequence having less than or equal to 5 total substitutions, deletions, and/or insertions in the amino acid sequence as set forth SEQ ID NO: 48. In some cases, the antibody provided herein comprises a heavy chain variable region comprising an amino acid sequence having less than or equal to 3 total substitutions, deletions, and/or insertions in the amino acid sequence as set forth SEQ ID NO: 44, and a light chain variable region comprising an amino acid sequence having less than or equal to 3 total substitutions, deletions, and/or insertions in the amino acid sequence as set forth SEQ ID NO: 48. In some cases, the antibody provided herein comprises a heavy chain variable region comprising an amino acid sequence having less than or equal to 2 total substitutions, deletions, and/or insertions in the amino acid sequence as set forth SEQ ID NO: 44, and a light chain variable region comprising an amino acid sequence having less than or equal to 2 total substitutions, deletions, and/or insertions in the amino acid sequence as set forth SEQ ID NO: 48. In some cases, the antibody provided herein comprises a heavy chain variable region comprising an amino acid sequence having less than or equal to 1 total substitutions, deletions, and/or insertions in the amino acid sequence as set forth SEQ ID NO: 44, and a light chain variable region comprising an amino acid sequence having less than or equal to 1 total substitutions, deletions, and/or insertions in the amino acid sequence as set forth SEQ ID NO: 48

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 44, and a light chain variable region comprising an amino acid sequence having at least 90%, 95%, 96%, 97%, 98%, 99%, or 100% sequence identity to the amino acid sequence as set forth SEQ ID NO: 49. In some cases, the antibody provided herein comprises a heavy chain variable region comprising an amino acid sequence having at least 95% sequence identity to the amino acid sequence as set forth SEQ ID NO: 44, and a light chain variable region comprising an amino acid sequence having at least 95% sequence identity to the amino acid sequence as set forth SEQ ID NO: 49. In some cases, the antibody provided herein comprises a heavy chain variable region comprising an amino acid sequence having at least 97% sequence identity to the amino acid sequence as set forth SEQ ID NO: 44, and a light chain variable region comprising an amino acid sequence having at least 97% sequence identity to the amino acid sequence as set forth SEQ ID NO: 49. In some cases, the antibody provided herein comprises a heavy chain variable region comprising an amino acid sequence having at least 99% sequence identity to the amino acid sequence as set forth SEQ ID NO: 44, and a light chain variable region comprising an amino acid sequence having at least 99% sequence identity to the amino acid sequence as set forth SEQ ID NO: 49. In some cases, the antibody provided herein comprises a heavy chain variable region comprising an amino acid sequence having 100% sequence identity to the amino acid sequence as set forth SEQ ID NO: 44, and a light chain variable region comprising an amino acid sequence having 100% sequence identity to the amino acid sequence as set forth SEQ ID NO: 49. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 44, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 49. In some cases, the antibody provided herein comprises a heavy chain variable region comprising an amino acid sequence having less than or equal to 10 total substitutions, deletions, and/or insertions in the amino acid sequence as set forth SEQ ID NO: 44, and a light chain variable region comprising an amino acid sequence having less than or equal to 10 total substitutions, deletions, and/or insertions in the amino acid sequence as set forth SEQ ID NO: 49. In some cases, the antibody provided herein comprises a heavy chain variable region comprising an amino acid sequence having less than or equal to 7 total substitutions, deletions, and/or insertions in the amino acid sequence as set forth SEQ ID NO: 44, and a light chain variable region comprising an amino acid sequence having less than or equal to 7 total substitutions, deletions, and/or insertions in the amino acid sequence as set forth SEQ ID NO: 49. In some cases, the antibody provided herein comprises a heavy chain variable region comprising an amino acid sequence having less than or equal to 5 total substitutions, deletions, and/or insertions in the amino acid sequence as set forth SEQ ID NO: 44, and a light chain variable region comprising an amino acid sequence having less than or equal to 5 total substitutions, deletions, and/or insertions in the amino acid sequence as set forth SEQ ID NO: 49. In some cases, the antibody provided herein comprises a heavy chain variable region comprising an amino acid sequence having less than or equal to 3 total substitutions, deletions, and/or insertions in the amino acid sequence as set forth SEQ ID NO: 44, and a light chain variable region comprising an amino acid sequence having less than or equal to 3 total substitutions, deletions, and/or insertions in the amino acid sequence as set forth SEQ ID NO: 49. In some cases, the antibody provided herein comprises a heavy chain variable region comprising an amino acid sequence having less than or equal to 2 total substitutions, deletions, and/or insertions in the amino acid sequence as set forth SEQ ID NO: 44, and a light chain variable region comprising an amino acid sequence having less than or equal to 2 total substitutions, deletions, and/or insertions in the amino acid sequence as set forth SEQ ID NO: 49. In some cases, the antibody provided herein comprises a heavy chain variable region comprising an amino acid sequence having less than or equal to 1 total substitutions, deletions, and/or insertions in the amino acid sequence as set forth SEQ ID NO: 44, and a light chain variable region comprising an amino acid sequence having less than or equal to 1 total substitutions, deletions, and/or insertions in the amino acid sequence as set forth SEQ ID NO: 49.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 44, and a light chain variable region comprising an amino acid sequence having at least 90%, 95%, 96%, 97%, 98%, 99%, or 100% sequence identity to the amino acid sequence as set forth SEQ ID NO: 50. In some cases, the antibody provided herein comprises a heavy chain variable region comprising an amino acid sequence having at least 95% sequence identity to the amino acid sequence as set forth SEQ ID NO: 44, and a light chain variable region comprising an amino acid sequence having at least 95% sequence identity to the amino acid sequence as set forth SEQ ID NO: 50. In some cases, the antibody provided herein comprises a heavy chain variable region comprising an amino acid sequence having at least 97% sequence identity to the amino acid sequence as set forth SEQ ID NO: 44, and a light chain variable region comprising an amino acid sequence having at least 97% sequence identity to the amino acid sequence as set forth SEQ ID NO: 50. In some cases, the antibody provided herein comprises a heavy chain variable region comprising an amino acid sequence having at least 99% sequence identity to the amino acid sequence as set forth SEQ ID NO: 44, and a light chain variable region comprising an amino acid sequence having at least 99% sequence identity to the amino acid sequence as set forth SEQ ID NO: 50. In some cases, the antibody provided herein comprises a heavy chain variable region comprising an amino acid sequence having 100% sequence identity to the amino acid sequence as set forth SEQ ID NO: 44, and a light chain variable region comprising an amino acid sequence having 100% sequence identity to the amino acid sequence as set forth SEQ ID NO: 50. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 44, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 50. In some cases, the antibody provided herein comprises a heavy chain variable region comprising an amino acid sequence having less than or equal to 10 total substitutions, deletions, and/or insertions in the amino acid sequence as set forth SEQ ID NO: 44, and a light chain variable region comprising an amino acid sequence having less than or equal to 10 total substitutions, deletions, and/or insertions in the amino acid sequence as set forth SEQ ID NO: 50. In some cases, the antibody provided herein comprises a heavy chain variable region comprising an amino acid sequence having less than or equal to 7 total substitutions, deletions, and/or insertions in the amino acid sequence as set forth SEQ ID NO: 44, and a light chain variable region comprising an amino acid sequence having less than or equal to 7 total substitutions, deletions, and/or insertions in the amino acid sequence as set forth SEQ ID NO: 50. In some cases, the antibody provided herein comprises a heavy chain variable region comprising an amino acid sequence having less than or equal to 5 total substitutions, deletions, and/or insertions in the amino acid sequence as set forth SEQ ID NO: 44, and a light chain variable region comprising an amino acid sequence having less than or equal to 5 total substitutions, deletions, and/or insertions in the amino acid sequence as set forth SEQ ID NO: 50. In some cases, the antibody provided herein comprises a heavy chain variable region comprising an amino acid sequence having less than or equal to 3 total substitutions, deletions, and/or insertions in the amino acid sequence as set forth SEQ ID NO: 44, and a light chain variable region comprising an amino acid sequence having less than or equal to 3 total substitutions, deletions, and/or insertions in the amino acid sequence as set forth SEQ ID NO: 50. In some cases, the antibody provided herein comprises a heavy chain variable region comprising an amino acid sequence having less than or equal to 2 total substitutions, deletions, and/or insertions in the amino acid sequence as set forth SEQ ID NO: 44, and a light chain variable region comprising an amino acid sequence having less than or equal to 2 total substitutions, deletions, and/or insertions in the amino acid sequence as set forth SEQ ID NO: 50. In some cases, the antibody provided herein comprises a heavy chain variable region comprising an amino acid sequence having less than or equal to 1 total substitutions, deletions, and/or insertions in the amino acid sequence as set forth SEQ ID NO: 44, and a light chain variable region comprising an amino acid sequence having less than or equal to 1 total substitutions, deletions, and/or insertions in the amino acid sequence as set forth SEQ ID NO: 50.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 44, and a light chain variable region comprising an amino acid sequence having at least 90%, 95%, 96%, 97%, 98%, 99%, or 100% sequence identity to the amino acid sequence as set forth SEQ ID NO: 51. In some cases, the antibody provided herein comprises a heavy chain variable region comprising an amino acid sequence having at least 95% sequence identity to the amino acid sequence as set forth SEQ ID NO: 44, and a light chain variable region comprising an amino acid sequence having at least 95% sequence identity to the amino acid sequence as set forth SEQ ID NO: 51. In some cases, the antibody provided herein comprises a heavy chain variable region comprising an amino acid sequence having at least 97% sequence identity to the amino acid sequence as set forth SEQ ID NO: 44, and a light chain variable region comprising an amino acid sequence having at least 97% sequence identity to the amino acid sequence as set forth SEQ ID NO: 51. In some cases, the antibody provided herein comprises a heavy chain variable region comprising an amino acid sequence having at least 99% sequence identity to the amino acid sequence as set forth SEQ ID NO: 44, and a light chain variable region comprising an amino acid sequence having at least 99% sequence identity to the amino acid sequence as set forth SEQ ID NO: 51. In some cases, the antibody provided herein comprises a heavy chain variable region comprising an amino acid sequence having 100% sequence identity to the amino acid sequence as set forth SEQ ID NO: 44, and a light chain variable region comprising an amino acid sequence having 100% sequence identity to the amino acid sequence as set forth SEQ ID NO: 51. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 44, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 51. In some cases, the antibody provided herein comprises a heavy chain variable region comprising an amino acid sequence having less than or equal to 10 total substitutions, deletions, and/or insertions in the amino acid sequence as set forth SEQ ID NO: 44, and a light chain variable region comprising an amino acid sequence having less than or equal to 10 total substitutions, deletions, and/or insertions in the amino acid sequence as set forth SEQ ID NO: 51. In some cases, the antibody provided herein comprises a heavy chain variable region comprising an amino acid sequence having less than or equal to 7 total substitutions, deletions, and/or insertions in the amino acid sequence as set forth SEQ ID NO: 44, and a light chain variable region comprising an amino acid sequence having less than or equal to 7 total substitutions, deletions, and/or insertions in the amino acid sequence as set forth SEQ ID NO: 51. In some cases, the antibody provided herein comprises a heavy chain variable region comprising an amino acid sequence having less than or equal to 5 total substitutions, deletions, and/or insertions in the amino acid sequence as set forth SEQ ID NO: 44, and a light chain variable region comprising an amino acid sequence having less than or equal to 5 total substitutions, deletions, and/or insertions in the amino acid sequence as set forth SEQ ID NO: 51. In some cases, the antibody provided herein comprises a heavy chain variable region comprising an amino acid sequence having less than or equal to 3 total substitutions, deletions, and/or insertions in the amino acid sequence as set forth SEQ ID NO: 44, and a light chain variable region comprising an amino acid sequence having less than or equal to 3 total substitutions, deletions, and/or insertions in the amino acid sequence as set forth SEQ ID NO: 51. In some cases, the antibody provided herein comprises a heavy chain variable region comprising an amino acid sequence having less than or equal to 2 total substitutions, deletions, and/or insertions in the amino acid sequence as set forth SEQ ID NO: 44, and a light chain variable region comprising an amino acid sequence having less than or equal to 2 total substitutions, deletions, and/or insertions in the amino acid sequence as set forth SEQ ID NO: 51. In some cases, the antibody provided herein comprises a heavy chain variable region comprising an amino acid sequence having less than or equal to 1 total substitutions, deletions, and/or insertions in the amino acid sequence as set forth SEQ ID NO: 44, and a light chain variable region comprising an amino acid sequence having less than or equal to 1 total substitutions, deletions, and/or insertions in the amino acid sequence as set forth SEQ ID NO: 51

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 44, and a light chain variable region comprising an amino acid sequence having at least 90%, 95%, 96%, 97%, 98%, 99%, or 100% sequence identity to the amino acid sequence as set forth SEQ ID NO: 52. In some cases, the antibody provided herein comprises a heavy chain variable region comprising an amino acid sequence having at least 95% sequence identity to the amino acid sequence as set forth SEQ ID NO: 44, and a light chain variable region comprising an amino acid sequence having at least 95% sequence identity to the amino acid sequence as set forth SEQ ID NO: 52. In some cases, the antibody provided herein comprises a heavy chain variable region comprising an amino acid sequence having at least 97% sequence identity to the amino acid sequence as set forth SEQ ID NO: 44, and a light chain variable region comprising an amino acid sequence having at least 97% sequence identity to the amino acid sequence as set forth SEQ ID NO: 52. In some cases, the antibody provided herein comprises a heavy chain variable region comprising an amino acid sequence having at least 99% sequence identity to the amino acid sequence as set forth SEQ ID NO: 44, and a light chain variable region comprising an amino acid sequence having at least 99% sequence identity to the amino acid sequence as set forth SEQ ID NO: 52. In some cases, the antibody provided herein comprises a heavy chain variable region comprising an amino acid sequence having 100% sequence identity to the amino acid sequence as set forth SEQ ID NO: 44, and a light chain variable region comprising an amino acid sequence having 100% sequence identity to the amino acid sequence as set forth SEQ ID NO: 52. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 44, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 52. In some cases, the antibody provided herein comprises a heavy chain variable region comprising an amino acid sequence having less than or equal to 10 total substitutions, deletions, and/or insertions in the amino acid sequence as set forth SEQ ID NO: 44, and a light chain variable region comprising an amino acid sequence having less than or equal to 10 total substitutions, deletions, and/or insertions in the amino acid sequence as set forth SEQ ID NO: 52. In some cases, the antibody provided herein comprises a heavy chain variable region comprising an amino acid sequence having less than or equal to 7 total substitutions, deletions, and/or insertions in the amino acid sequence as set forth SEQ ID NO: 44, and a light chain variable region comprising an amino acid sequence having less than or equal to 7 total substitutions, deletions, and/or insertions in the amino acid sequence as set forth SEQ ID NO: 52. In some cases, the antibody provided herein comprises a heavy chain variable region comprising an amino acid sequence having less than or equal to 5 total substitutions, deletions, and/or insertions in the amino acid sequence as set forth SEQ ID NO: 44, and a light chain variable region comprising an amino acid sequence having less than or equal to 5 total substitutions, deletions, and/or insertions in the amino acid sequence as set forth SEQ ID NO: 52. In some cases, the antibody provided herein comprises a heavy chain variable region comprising an amino acid sequence having less than or equal to 3 total substitutions, deletions, and/or insertions in the amino acid sequence as set forth SEQ ID NO: 44, and a light chain variable region comprising an amino acid sequence having less than or equal to 3 total substitutions, deletions, and/or insertions in the amino acid sequence as set forth SEQ ID NO: 52. In some cases, the antibody provided herein comprises a heavy chain variable region comprising an amino acid sequence having less than or equal to 2 total substitutions, deletions, and/or insertions in the amino acid sequence as set forth SEQ ID NO: 44, and a light chain variable region comprising an amino acid sequence having less than or equal to 2 total substitutions, deletions, and/or insertions in the amino acid sequence as set forth SEQ ID NO: 52. In some cases, the antibody provided herein comprises a heavy chain variable region comprising an amino acid sequence having less than or equal to 1 total substitutions, deletions, and/or insertions in the amino acid sequence as set forth SEQ ID NO: 44, and a light chain variable region comprising an amino acid sequence having less than or equal to 1 total substitutions, deletions, and/or insertions in the amino acid sequence as set forth SEQ ID NO: 52.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 44, and a light chain variable region comprising an amino acid sequence having at least 90%, 95%, 96%, 97%, 98%, 99%, or 100% sequence identity to the amino acid sequence as set forth SEQ ID NO: 65. In some cases, the antibody provided herein comprises a heavy chain variable region comprising an amino acid sequence having at least 95% sequence identity to the amino acid sequence as set forth SEQ ID NO: 44, and a light chain variable region comprising an amino acid sequence having at least 95% sequence identity to the amino acid sequence as set forth SEQ ID NO: 65. In some cases, the antibody provided herein comprises a heavy chain variable region comprising an amino acid sequence having at least 97% sequence identity to the amino acid sequence as set forth SEQ ID NO: 44, and a light chain variable region comprising an amino acid sequence having at least 97% sequence identity to the amino acid sequence as set forth SEQ ID NO: 65. In some cases, the antibody provided herein comprises a heavy chain variable region comprising an amino acid sequence having at least 99% sequence identity to the amino acid sequence as set forth SEQ ID NO: 44, and a light chain variable region comprising an amino acid sequence having at least 99% sequence identity to the amino acid sequence as set forth SEQ ID NO: 65. In some cases, the antibody provided herein comprises a heavy chain variable region comprising an amino acid sequence having 100% sequence identity to the amino acid sequence as set forth SEQ ID NO: 44, and a light chain variable region comprising an amino acid sequence having 100% sequence identity to the amino acid sequence as set forth SEQ ID NO: 65. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 44, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 65. In some cases, the antibody provided herein comprises a heavy chain variable region comprising an amino acid sequence having less than or equal to 10 total substitutions, deletions, and/or insertions in the amino acid sequence as set forth SEQ ID NO: 44, and a light chain variable region comprising an amino acid sequence having less than or equal to 10 total substitutions, deletions, and/or insertions in the amino acid sequence as set forth SEQ ID NO: 65. In some cases, the antibody provided herein comprises a heavy chain variable region comprising an amino acid sequence having less than or equal to 7 total substitutions, deletions, and/or insertions in the amino acid sequence as set forth SEQ ID NO: 44, and a light chain variable region comprising an amino acid sequence having less than or equal to 7 total substitutions, deletions, and/or insertions in the amino acid sequence as set forth SEQ ID NO: 65. In some cases, the antibody provided herein comprises a heavy chain variable region comprising an amino acid sequence having less than or equal to 5 total substitutions, deletions, and/or insertions in the amino acid sequence as set forth SEQ ID NO: 44, and a light chain variable region comprising an amino acid sequence having less than or equal to 5 total substitutions, deletions, and/or insertions in the amino acid sequence as set forth SEQ ID NO: 65. In some cases, the antibody provided herein comprises a heavy chain variable region comprising an amino acid sequence having less than or equal to 3 total substitutions, deletions, and/or insertions in the amino acid sequence as set forth SEQ ID NO: 44, and a light chain variable region comprising an amino acid sequence having less than or equal to 3 total substitutions, deletions, and/or insertions in the amino acid sequence as set forth SEQ ID NO: 65. In some cases, the antibody provided herein comprises a heavy chain variable region comprising an amino acid sequence having less than or equal to 2 total substitutions, deletions, and/or insertions in the amino acid sequence as set forth SEQ ID NO: 44, and a light chain variable region comprising an amino acid sequence having less than or equal to 2 total substitutions, deletions, and/or insertions in the amino acid sequence as set forth SEQ ID NO: 65. In some cases, the antibody provided herein comprises a heavy chain variable region comprising an amino acid sequence having less than or equal to 1 total substitutions, deletions, and/or insertions in the amino acid sequence as set forth SEQ ID NO: 44, and a light chain variable region comprising an amino acid sequence having less than or equal to 1 total substitutions, deletions, and/or insertions in the amino acid sequence as set forth SEQ ID NO: 65.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 44, and a light chain variable region comprising an amino acid sequence having at least 90%, 95%, 96%, 97%, 98%, 99%, or 100% sequence identity to the amino acid sequence as set forth SEQ ID NO: 66. In some cases, the antibody provided herein comprises a heavy chain variable region comprising an amino acid sequence having at least 95% sequence identity to the amino acid sequence as set forth SEQ ID NO: 44, and a light chain variable region comprising an amino acid sequence having at least 95% sequence identity to the amino acid sequence as set forth SEQ ID NO: 66. In some cases, the antibody provided herein comprises a heavy chain variable region comprising an amino acid sequence having at least 97% sequence identity to the amino acid sequence as set forth SEQ ID NO: 44, and a light chain variable region comprising an amino acid sequence having at least 97% sequence identity to the amino acid sequence as set forth SEQ ID NO: 66. In some cases, the antibody provided herein comprises a heavy chain variable region comprising an amino acid sequence having at least 99% sequence identity to the amino acid sequence as set forth SEQ ID NO: 44, and a light chain variable region comprising an amino acid sequence having at least 99% sequence identity to the amino acid sequence as set forth SEQ ID NO: 66. In some cases, the antibody provided herein comprises a heavy chain variable region comprising an amino acid sequence having 100% sequence identity to the amino acid sequence as set forth SEQ ID NO: 44, and a light chain variable region comprising an amino acid sequence having 100% sequence identity to the amino acid sequence as set forth SEQ ID NO: 66. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 44, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 66. In some cases, the antibody provided herein comprises a heavy chain variable region comprising an amino acid sequence having less than or equal to 10 total substitutions, deletions, and/or insertions in the amino acid sequence as set forth SEQ ID NO: 44, and a light chain variable region comprising an amino acid sequence having less than or equal to 10 total substitutions, deletions, and/or insertions in the amino acid sequence as set forth SEQ ID NO: 66. In some cases, the antibody provided herein comprises a heavy chain variable region comprising an amino acid sequence having less than or equal to 7 total substitutions, deletions, and/or insertions in the amino acid sequence as set forth SEQ ID NO: 44, and a light chain variable region comprising an amino acid sequence having less than or equal to 7 total substitutions, deletions, and/or insertions in the amino acid sequence as set forth SEQ ID NO: 66. In some cases, the antibody provided herein comprises a heavy chain variable region comprising an amino acid sequence having less than or equal to 5 total substitutions, deletions, and/or insertions in the amino acid sequence as set forth SEQ ID NO: 44, and a light chain variable region comprising an amino acid sequence having less than or equal to 5 total substitutions, deletions, and/or insertions in the amino acid sequence as set forth SEQ ID NO: 66. In some cases, the antibody provided herein comprises a heavy chain variable region comprising an amino acid sequence having less than or equal to 3 total substitutions, deletions, and/or insertions in the amino acid sequence as set forth SEQ ID NO: 44, and a light chain variable region comprising an amino acid sequence having less than or equal to 3 total substitutions, deletions, and/or insertions in the amino acid sequence as set forth SEQ ID NO: 66. In some cases, the antibody provided herein comprises a heavy chain variable region comprising an amino acid sequence having less than or equal to 2 total substitutions, deletions, and/or insertions in the amino acid sequence as set forth SEQ ID NO: 44, and a light chain variable region comprising an amino acid sequence having less than or equal to 2 total substitutions, deletions, and/or insertions in the amino acid sequence as set forth SEQ ID NO: 66. In some cases, the antibody provided herein comprises a heavy chain variable region comprising an amino acid sequence having less than or equal to 1 total substitutions, deletions, and/or insertions in the amino acid sequence as set forth SEQ ID NO: 44, and a light chain variable region comprising an amino acid sequence having less than or equal to 1 total substitutions, deletions, and/or insertions in the amino acid sequence as set forth SEQ ID NO: 66.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 45, and a light chain variable region comprising an amino acid sequence having at least 90%, 95%, 96%, 97%, 98%, 99%, or 100% sequence identity to the amino acid sequence as set forth SEQ ID NO: 2. In some cases, the antibody provided herein comprises a heavy chain variable region comprising an amino acid sequence having at least 95% sequence identity to the amino acid sequence as set forth SEQ ID NO: 45, and a light chain variable region comprising an amino acid sequence having at least 95% sequence identity to the amino acid sequence as set forth SEQ ID NO: 2. In some cases, the antibody provided herein comprises a heavy chain variable region comprising an amino acid sequence having at least 97% sequence identity to the amino acid sequence as set forth SEQ ID NO: 45, and a light chain variable region comprising an amino acid sequence having at least 97% sequence identity to the amino acid sequence as set forth SEQ ID NO: 2. In some cases, the antibody provided herein comprises a heavy chain variable region comprising an amino acid sequence having at least 99% sequence identity to the amino acid sequence as set forth SEQ ID NO: 45, and a light chain variable region comprising an amino acid sequence having at least 99% sequence identity to the amino acid sequence as set forth SEQ ID NO: 2. In some cases, the antibody provided herein comprises a heavy chain variable region comprising an amino acid sequence having 100% sequence identity to the amino acid sequence as set forth SEQ ID NO: 45, and a light chain variable region comprising an amino acid sequence having 100% sequence identity to the amino acid sequence as set forth SEQ ID NO: 2. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 45, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 2. In some cases, the antibody provided herein comprises a heavy chain variable region comprising an amino acid sequence having less than or equal to 10 total substitutions, deletions, and/or insertions in the amino acid sequence as set forth SEQ ID NO: 45, and a light chain variable region comprising an amino acid sequence having less than or equal to 10 total substitutions, deletions, and/or insertions in the amino acid sequence as set forth SEQ ID NO: 2. In some cases, the antibody provided herein comprises a heavy chain variable region comprising an amino acid sequence having less than or equal to 7 total substitutions, deletions, and/or insertions in the amino acid sequence as set forth SEQ ID NO: 45, and a light chain variable region comprising an amino acid sequence having less than or equal to 7 total substitutions, deletions, and/or insertions in the amino acid sequence as set forth SEQ ID NO: 2. In some cases, the antibody provided herein comprises a heavy chain variable region comprising an amino acid sequence having less than or equal to 5 total substitutions, deletions, and/or insertions in the amino acid sequence as set forth SEQ ID NO: 45, and a light chain variable region comprising an amino acid sequence having less than or equal to 5 total substitutions, deletions, and/or insertions in the amino acid sequence as set forth SEQ ID NO: 2. In some cases, the antibody provided herein comprises a heavy chain variable region comprising an amino acid sequence having less than or equal to 3 total substitutions, deletions, and/or insertions in the amino acid sequence as set forth SEQ ID NO: 45, and a light chain variable region comprising an amino acid sequence having less than or equal to 3 total substitutions, deletions, and/or insertions in the amino acid sequence as set forth SEQ ID NO: 2. In some cases, the antibody provided herein comprises a heavy chain variable region comprising an amino acid sequence having less than or equal to 2 total substitutions, deletions, and/or insertions in the amino acid sequence as set forth SEQ ID NO: 45, and a light chain variable region comprising an amino acid sequence having less than or equal to 2 total substitutions, deletions, and/or insertions in the amino acid sequence as set forth SEQ ID NO: 2. In some cases, the antibody provided herein comprises a heavy chain variable region comprising an amino acid sequence having less than or equal to 1 total substitutions, deletions, and/or insertions in the amino acid sequence as set forth SEQ ID NO: 45, and a light chain variable region comprising an amino acid sequence having less than or equal to 1 total substitutions, deletions, and/or insertions in the amino acid sequence as set forth SEQ ID NO: 2.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 45, and a light chain variable region comprising an amino acid sequence having at least 90%, 95%, 96%, 97%, 98%, 99%, or 100% sequence identity to the amino acid sequence as set forth SEQ ID NO: 48. In some cases, the antibody provided herein comprises a heavy chain variable region comprising an amino acid sequence having at least 95% sequence identity to the amino acid sequence as set forth SEQ ID NO: 45, and a light chain variable region comprising an amino acid sequence having at least 95% sequence identity to the amino acid sequence as set forth SEQ ID NO: 48. In some cases, the antibody provided herein comprises a heavy chain variable region comprising an amino acid sequence having at least 97% sequence identity to the amino acid sequence as set forth SEQ ID NO: 45, and a light chain variable region comprising an amino acid sequence having at least 97% sequence identity to the amino acid sequence as set forth SEQ ID NO: 48. In some cases, the antibody provided herein comprises a heavy chain variable region comprising an amino acid sequence having at least 99% sequence identity to the amino acid sequence as set forth SEQ ID NO: 45, and a light chain variable region comprising an amino acid sequence having at least 99% sequence identity to the amino acid sequence as set forth SEQ ID NO: 48. In some cases, the antibody provided herein comprises a heavy chain variable region comprising an amino acid sequence having 100% sequence identity to the amino acid sequence as set forth SEQ ID NO: 45, and a light chain variable region comprising an amino acid sequence having 100% sequence identity to the amino acid sequence as set forth SEQ ID NO: 48. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 45, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 48. In some cases, the antibody provided herein comprises a heavy chain variable region comprising an amino acid sequence having less than or equal to 10 total substitutions, deletions, and/or insertions in the amino acid sequence as set forth SEQ ID NO: 45, and a light chain variable region comprising an amino acid sequence having less than or equal to 10 total substitutions, deletions, and/or insertions in the amino acid sequence as set forth SEQ ID NO: 48. In some cases, the antibody provided herein comprises a heavy chain variable region comprising an amino acid sequence having less than or equal to 7 total substitutions, deletions, and/or insertions in the amino acid sequence as set forth SEQ ID NO: 45, and a light chain variable region comprising an amino acid sequence having less than or equal to 7 total substitutions, deletions, and/or insertions in the amino acid sequence as set forth SEQ ID NO: 48. In some cases, the antibody provided herein comprises a heavy chain variable region comprising an amino acid sequence having less than or equal to 5 total substitutions, deletions, and/or insertions in the amino acid sequence as set forth SEQ ID NO: 45, and a light chain variable region comprising an amino acid sequence having less than or equal to 5 total substitutions, deletions, and/or insertions in the amino acid sequence as set forth SEQ ID NO: 48. In some cases, the antibody provided herein comprises a heavy chain variable region comprising an amino acid sequence having less than or equal to 3 total substitutions, deletions, and/or insertions in the amino acid sequence as set forth SEQ ID NO: 45, and a light chain variable region comprising an amino acid sequence having less than or equal to 3 total substitutions, deletions, and/or insertions in the amino acid sequence as set forth SEQ ID NO: 48. In some cases, the antibody provided herein comprises a heavy chain variable region comprising an amino acid sequence having less than or equal to 2 total substitutions, deletions, and/or insertions in the amino acid sequence as set forth SEQ ID NO: 45, and a light chain variable region comprising an amino acid sequence having less than or equal to 2 total substitutions, deletions, and/or insertions in the amino acid sequence as set forth SEQ ID NO: 48. In some cases, the antibody provided herein comprises a heavy chain variable region comprising an amino acid sequence having less than or equal to 1 total substitutions, deletions, and/or insertions in the amino acid sequence as set forth SEQ ID NO: 45, and a light chain variable region comprising an amino acid sequence having less than or equal to 1 total substitutions, deletions, and/or insertions in the amino acid sequence as set forth SEQ ID NO: 48

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 45, and a light chain variable region comprising an amino acid sequence having at least 90%, 95%, 96%, 97%, 98%, 99%, or 100% sequence identity to the amino acid sequence as set forth SEQ ID NO: 49. In some cases, the antibody provided herein comprises a heavy chain variable region comprising an amino acid sequence having at least 95% sequence identity to the amino acid sequence as set forth SEQ ID NO: 45, and a light chain variable region comprising an amino acid sequence having at least 95% sequence identity to the amino acid sequence as set forth SEQ ID NO: 49. In some cases, the antibody provided herein comprises a heavy chain variable region comprising an amino acid sequence having at least 97% sequence identity to the amino acid sequence as set forth SEQ ID NO: 45, and a light chain variable region comprising an amino acid sequence having at least 97% sequence identity to the amino acid sequence as set forth SEQ ID NO: 49. In some cases, the antibody provided herein comprises a heavy chain variable region comprising an amino acid sequence having at least 99% sequence identity to the amino acid sequence as set forth SEQ ID NO: 45, and a light chain variable region comprising an amino acid sequence having at least 99% sequence identity to the amino acid sequence as set forth SEQ ID NO: 49. In some cases, the antibody provided herein comprises a heavy chain variable region comprising an amino acid sequence having 100% sequence identity to the amino acid sequence as set forth SEQ ID NO: 45, and a light chain variable region comprising an amino acid sequence having 100% sequence identity to the amino acid sequence as set forth SEQ ID NO: 49. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 45, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 49. In some cases, the antibody provided herein comprises a heavy chain variable region comprising an amino acid sequence having less than or equal to 10 total substitutions, deletions, and/or insertions in the amino acid sequence as set forth SEQ ID NO: 45, and a light chain variable region comprising an amino acid sequence having less than or equal to 10 total substitutions, deletions, and/or insertions in the amino acid sequence as set forth SEQ ID NO: 49. In some cases, the antibody provided herein comprises a heavy chain variable region comprising an amino acid sequence having less than or equal to 7 total substitutions, deletions, and/or insertions in the amino acid sequence as set forth SEQ ID NO: 45, and a light chain variable region comprising an amino acid sequence having less than or equal to 7 total substitutions, deletions, and/or insertions in the amino acid sequence as set forth SEQ ID NO: 49. In some cases, the antibody provided herein comprises a heavy chain variable region comprising an amino acid sequence having less than or equal to 5 total substitutions, deletions, and/or insertions in the amino acid sequence as set forth SEQ ID NO: 45, and a light chain variable region comprising an amino acid sequence having less than or equal to 5 total substitutions, deletions, and/or insertions in the amino acid sequence as set forth SEQ ID NO: 49. In some cases, the antibody provided herein comprises a heavy chain variable region comprising an amino acid sequence having less than or equal to 3 total substitutions, deletions, and/or insertions in the amino acid sequence as set forth SEQ ID NO: 45, and a light chain variable region comprising an amino acid sequence having less than or equal to 3 total substitutions, deletions, and/or insertions in the amino acid sequence as set forth SEQ ID NO: 49. In some cases, the antibody provided herein comprises a heavy chain variable region comprising an amino acid sequence having less than or equal to 2 total substitutions, deletions, and/or insertions in the amino acid sequence as set forth SEQ ID NO: 45, and a light chain variable region comprising an amino acid sequence having less than or equal to 2 total substitutions, deletions, and/or insertions in the amino acid sequence as set forth SEQ ID NO: 49. In some cases, the antibody provided herein comprises a heavy chain variable region comprising an amino acid sequence having less than or equal to 1 total substitutions, deletions, and/or insertions in the amino acid sequence as set forth SEQ ID NO: 45, and a light chain variable region comprising an amino acid sequence having less than or equal to 1 total substitutions, deletions, and/or insertions in the amino acid sequence as set forth SEQ ID NO: 49.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 45, and a light chain variable region comprising an amino acid sequence having at least 90%, 95%, 96%, 97%, 98%, 99%, or 100% sequence identity to the amino acid sequence as set forth SEQ ID NO: 50. In some cases, the antibody provided herein comprises a heavy chain variable region comprising an amino acid sequence having at least 95% sequence identity to the amino acid sequence as set forth SEQ ID NO: 45, and a light chain variable region comprising an amino acid sequence having at least 95% sequence identity to the amino acid sequence as set forth SEQ ID NO: 50. In some cases, the antibody provided herein comprises a heavy chain variable region comprising an amino acid sequence having at least 97% sequence identity to the amino acid sequence as set forth SEQ ID NO: 45, and a light chain variable region comprising an amino acid sequence having at least 97% sequence identity to the amino acid sequence as set forth SEQ ID NO: 50. In some cases, the antibody provided herein comprises a heavy chain variable region comprising an amino acid sequence having at least 99% sequence identity to the amino acid sequence as set forth SEQ ID NO: 45, and a light chain variable region comprising an amino acid sequence having at least 99% sequence identity to the amino acid sequence as set forth SEQ ID NO: 50. In some cases, the antibody provided herein comprises a heavy chain variable region comprising an amino acid sequence having 100% sequence identity to the amino acid sequence as set forth SEQ ID NO: 45, and a light chain variable region comprising an amino acid sequence having 100% sequence identity to the amino acid sequence as set forth SEQ ID NO: 50. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 45, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 50. In some cases, the antibody provided herein comprises a heavy chain variable region comprising an amino acid sequence having less than or equal to 10 total substitutions, deletions, and/or insertions in the amino acid sequence as set forth SEQ ID NO: 45, and a light chain variable region comprising an amino acid sequence having less than or equal to 10 total substitutions, deletions, and/or insertions in the amino acid sequence as set forth SEQ ID NO: 50. In some cases, the antibody provided herein comprises a heavy chain variable region comprising an amino acid sequence having less than or equal to 7 total substitutions, deletions, and/or insertions in the amino acid sequence as set forth SEQ ID NO: 45, and a light chain variable region comprising an amino acid sequence having less than or equal to 7 total substitutions, deletions, and/or insertions in the amino acid sequence as set forth SEQ ID NO: 50. In some cases, the antibody provided herein comprises a heavy chain variable region comprising an amino acid sequence having less than or equal to 5 total substitutions, deletions, and/or insertions in the amino acid sequence as set forth SEQ ID NO: 45, and a light chain variable region comprising an amino acid sequence having less than or equal to 5 total substitutions, deletions, and/or insertions in the amino acid sequence as set forth SEQ ID NO: 50. In some cases, the antibody provided herein comprises a heavy chain variable region comprising an amino acid sequence having less than or equal to 3 total substitutions, deletions, and/or insertions in the amino acid sequence as set forth SEQ ID NO: 45, and a light chain variable region comprising an amino acid sequence having less than or equal to 3 total substitutions, deletions, and/or insertions in the amino acid sequence as set forth SEQ ID NO: 50. In some cases, the antibody provided herein comprises a heavy chain variable region comprising an amino acid sequence having less than or equal to 2 total substitutions, deletions, and/or insertions in the amino acid sequence as set forth SEQ ID NO: 45, and a light chain variable region comprising an amino acid sequence having less than or equal to 2 total substitutions, deletions, and/or insertions in the amino acid sequence as set forth SEQ ID NO: 50. In some cases, the antibody provided herein comprises a heavy chain variable region comprising an amino acid sequence having less than or equal to 1 total substitutions, deletions, and/or insertions in the amino acid sequence as set forth SEQ ID NO: 45, and a light chain variable region comprising an amino acid sequence having less than or equal to 1 total substitutions, deletions, and/or insertions in the amino acid sequence as set forth SEQ ID NO: 50.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 45, and a light chain variable region comprising an amino acid sequence having at least 90%, 95%, 96%, 97%, 98%, 99%, or 100% sequence identity to the amino acid sequence as set forth SEQ ID NO: 51. In some cases, the antibody provided herein comprises a heavy chain variable region comprising an amino acid sequence having at least 95% sequence identity to the amino acid sequence as set forth SEQ ID NO: 45, and a light chain variable region comprising an amino acid sequence having at least 95% sequence identity to the amino acid sequence as set forth SEQ ID NO: 51. In some cases, the antibody provided herein comprises a heavy chain variable region comprising an amino acid sequence having at least 97% sequence identity to the amino acid sequence as set forth SEQ ID NO: 45, and a light chain variable region comprising an amino acid sequence having at least 97% sequence identity to the amino acid sequence as set forth SEQ ID NO: 51. In some cases, the antibody provided herein comprises a heavy chain variable region comprising an amino acid sequence having at least 99% sequence identity to the amino acid sequence as set forth SEQ ID NO: 45, and a light chain variable region comprising an amino acid sequence having at least 99% sequence identity to the amino acid sequence as set forth SEQ ID NO: 51. In some cases, the antibody provided herein comprises a heavy chain variable region comprising an amino acid sequence having 100% sequence identity to the amino acid sequence as set forth SEQ ID NO: 45, and a light chain variable region comprising an amino acid sequence having 100% sequence identity to the amino acid sequence as set forth SEQ ID NO: 51. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 45, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 51. In some cases, the antibody provided herein comprises a heavy chain variable region comprising an amino acid sequence having less than or equal to 10 total substitutions, deletions, and/or insertions in the amino acid sequence as set forth SEQ ID NO: 45, and a light chain variable region comprising an amino acid sequence having less than or equal to 10 total substitutions, deletions, and/or insertions in the amino acid sequence as set forth SEQ ID NO: 51. In some cases, the antibody provided herein comprises a heavy chain variable region comprising an amino acid sequence having less than or equal to 7 total substitutions, deletions, and/or insertions in the amino acid sequence as set forth SEQ ID NO: 45, and a light chain variable region comprising an amino acid sequence having less than or equal to 7 total substitutions, deletions, and/or insertions in the amino acid sequence as set forth SEQ ID NO: 51. In some cases, the antibody provided herein comprises a heavy chain variable region comprising an amino acid sequence having less than or equal to 5 total substitutions, deletions, and/or insertions in the amino acid sequence as set forth SEQ ID NO: 45, and a light chain variable region comprising an amino acid sequence having less than or equal to 5 total substitutions, deletions, and/or insertions in the amino acid sequence as set forth SEQ ID NO: 51. In some cases, the antibody provided herein comprises a heavy chain variable region comprising an amino acid sequence having less than or equal to 3 total substitutions, deletions, and/or insertions in the amino acid sequence as set forth SEQ ID NO: 45, and a light chain variable region comprising an amino acid sequence having less than or equal to 3 total substitutions, deletions, and/or insertions in the amino acid sequence as set forth SEQ ID NO: 51. In some cases, the antibody provided herein comprises a heavy chain variable region comprising an amino acid sequence having less than or equal to 2 total substitutions, deletions, and/or insertions in the amino acid sequence as set forth SEQ ID NO: 45, and a light chain variable region comprising an amino acid sequence having less than or equal to 2 total substitutions, deletions, and/or insertions in the amino acid sequence as set forth SEQ ID NO: 51. In some cases, the antibody provided herein comprises a heavy chain variable region comprising an amino acid sequence having less than or equal to 1 total substitutions, deletions, and/or insertions in the amino acid sequence as set forth SEQ ID NO: 45, and a light chain variable region comprising an amino acid sequence having less than or equal to 1 total substitutions, deletions, and/or insertions in the amino acid sequence as set forth SEQ ID NO: 51

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 45, and a light chain variable region comprising an amino acid sequence having at least 90%, 95%, 96%, 97%, 98%, 99%, or 100% sequence identity to the amino acid sequence as set forth SEQ ID NO: 52. In some cases, the antibody provided herein comprises a heavy chain variable region comprising an amino acid sequence having at least 95% sequence identity to the amino acid sequence as set forth SEQ ID NO: 45, and a light chain variable region comprising an amino acid sequence having at least 95% sequence identity to the amino acid sequence as set forth SEQ ID NO: 52. In some cases, the antibody provided herein comprises a heavy chain variable region comprising an amino acid sequence having at least 97% sequence identity to the amino acid sequence as set forth SEQ ID NO: 45, and a light chain variable region comprising an amino acid sequence having at least 97% sequence identity to the amino acid sequence as set forth SEQ ID NO: 52. In some cases, the antibody provided herein comprises a heavy chain variable region comprising an amino acid sequence having at least 99% sequence identity to the amino acid sequence as set forth SEQ ID NO: 45, and a light chain variable region comprising an amino acid sequence having at least 99% sequence identity to the amino acid sequence as set forth SEQ ID NO: 52. In some cases, the antibody provided herein comprises a heavy chain variable region comprising an amino acid sequence having 100% sequence identity to the amino acid sequence as set forth SEQ ID NO: 45, and a light chain variable region comprising an amino acid sequence having 100% sequence identity to the amino acid sequence as set forth SEQ ID NO: 52. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 45, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 52. In some cases, the antibody provided herein comprises a heavy chain variable region comprising an amino acid sequence having less than or equal to 10 total substitutions, deletions, and/or insertions in the amino acid sequence as set forth SEQ ID NO: 45, and a light chain variable region comprising an amino acid sequence having less than or equal to 10 total substitutions, deletions, and/or insertions in the amino acid sequence as set forth SEQ ID NO: 52. In some cases, the antibody provided herein comprises a heavy chain variable region comprising an amino acid sequence having less than or equal to 7 total substitutions, deletions, and/or insertions in the amino acid sequence as set forth SEQ ID NO: 45, and a light chain variable region comprising an amino acid sequence having less than or equal to 7 total substitutions, deletions, and/or insertions in the amino acid sequence as set forth SEQ ID NO: 52. In some cases, the antibody provided herein comprises a heavy chain variable region comprising an amino acid sequence having less than or equal to 5 total substitutions, deletions, and/or insertions in the amino acid sequence as set forth SEQ ID NO: 45, and a light chain variable region comprising an amino acid sequence having less than or equal to 5 total substitutions, deletions, and/or insertions in the amino acid sequence as set forth SEQ ID NO: 52. In some cases, the antibody provided herein comprises a heavy chain variable region comprising an amino acid sequence having less than or equal to 3 total substitutions, deletions, and/or insertions in the amino acid sequence as set forth SEQ ID NO: 45, and a light chain variable region comprising an amino acid sequence having less than or equal to 3 total substitutions, deletions, and/or insertions in the amino acid sequence as set forth SEQ ID NO: 52. In some cases, the antibody provided herein comprises a heavy chain variable region comprising an amino acid sequence having less than or equal to 2 total substitutions, deletions, and/or insertions in the amino acid sequence as set forth SEQ ID NO: 45, and a light chain variable region comprising an amino acid sequence having less than or equal to 2 total substitutions, deletions, and/or insertions in the amino acid sequence as set forth SEQ ID NO: 52. In some cases, the antibody provided herein comprises a heavy chain variable region comprising an amino acid sequence having less than or equal to 1 total substitutions, deletions, and/or insertions in the amino acid sequence as set forth SEQ ID NO: 45, and a light chain variable region comprising an amino acid sequence having less than or equal to 1 total substitutions, deletions, and/or insertions in the amino acid sequence as set forth SEQ ID NO: 52.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 45, and a light chain variable region comprising an amino acid sequence having at least 90%, 95%, 96%, 97%, 98%, 99%, or 100% sequence identity to the amino acid sequence as set forth SEQ ID NO: 65. In some cases, the antibody provided herein comprises a heavy chain variable region comprising an amino acid sequence having at least 95% sequence identity to the amino acid sequence as set forth SEQ ID NO: 45, and a light chain variable region comprising an amino acid sequence having at least 95% sequence identity to the amino acid sequence as set forth SEQ ID NO: 65. In some cases, the antibody provided herein comprises a heavy chain variable region comprising an amino acid sequence having at least 97% sequence identity to the amino acid sequence as set forth SEQ ID NO: 45, and a light chain variable region comprising an amino acid sequence having at least 97% sequence identity to the amino acid sequence as set forth SEQ ID NO: 65. In some cases, the antibody provided herein comprises a heavy chain variable region comprising an amino acid sequence having at least 99% sequence identity to the amino acid sequence as set forth SEQ ID NO: 45, and a light chain variable region comprising an amino acid sequence having at least 99% sequence identity to the amino acid sequence as set forth SEQ ID NO: 65. In some cases, the antibody provided herein comprises a heavy chain variable region comprising an amino acid sequence having 100% sequence identity to the amino acid sequence as set forth SEQ ID NO: 45, and a light chain variable region comprising an amino acid sequence having 100% sequence identity to the amino acid sequence as set forth SEQ ID NO: 65. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 45, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 65. In some cases, the antibody provided herein comprises a heavy chain variable region comprising an amino acid sequence having less than or equal to 10 total substitutions, deletions, and/or insertions in the amino acid sequence as set forth SEQ ID NO: 45, and a light chain variable region comprising an amino acid sequence having less than or equal to 10 total substitutions, deletions, and/or insertions in the amino acid sequence as set forth SEQ ID NO: 65. In some cases, the antibody provided herein comprises a heavy chain variable region comprising an amino acid sequence having less than or equal to 7 total substitutions, deletions, and/or insertions in the amino acid sequence as set forth SEQ ID NO: 45, and a light chain variable region comprising an amino acid sequence having less than or equal to 7 total substitutions, deletions, and/or insertions in the amino acid sequence as set forth SEQ ID NO: 65. In some cases, the antibody provided herein comprises a heavy chain variable region comprising an amino acid sequence having less than or equal to 5 total substitutions, deletions, and/or insertions in the amino acid sequence as set forth SEQ ID NO: 45, and a light chain variable region comprising an amino acid sequence having less than or equal to 5 total substitutions, deletions, and/or insertions in the amino acid sequence as set forth SEQ ID NO: 65. In some cases, the antibody provided herein comprises a heavy chain variable region comprising an amino acid sequence having less than or equal to 3 total substitutions, deletions, and/or insertions in the amino acid sequence as set forth SEQ ID NO: 45, and a light chain variable region comprising an amino acid sequence having less than or equal to 3 total substitutions, deletions, and/or insertions in the amino acid sequence as set forth SEQ ID NO: 65. In some cases, the antibody provided herein comprises a heavy chain variable region comprising an amino acid sequence having less than or equal to 2 total substitutions, deletions, and/or insertions in the amino acid sequence as set forth SEQ ID NO: 45, and a light chain variable region comprising an amino acid sequence having less than or equal to 2 total substitutions, deletions, and/or insertions in the amino acid sequence as set forth SEQ ID NO: 65. In some cases, the antibody provided herein comprises a heavy chain variable region comprising an amino acid sequence having less than or equal to 1 total substitutions, deletions, and/or insertions in the amino acid sequence as set forth SEQ ID NO: 45, and a light chain variable region comprising an amino acid sequence having less than or equal to 1 total substitutions, deletions, and/or insertions in the amino acid sequence as set forth SEQ ID NO: 65.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 45, and a light chain variable region comprising an amino acid sequence having at least 90%, 95%, 96%, 97%, 98%, 99%, or 100% sequence identity to the amino acid sequence as set forth SEQ ID NO: 66. In some cases, the antibody provided herein comprises a heavy chain variable region comprising an amino acid sequence having at least 95% sequence identity to the amino acid sequence as set forth SEQ ID NO: 45, and a light chain variable region comprising an amino acid sequence having at least 95% sequence identity to the amino acid sequence as set forth SEQ ID NO: 66. In some cases, the antibody provided herein comprises a heavy chain variable region comprising an amino acid sequence having at least 97% sequence identity to the amino acid sequence as set forth SEQ ID NO: 45, and a light chain variable region comprising an amino acid sequence having at least 97% sequence identity to the amino acid sequence as set forth SEQ ID NO: 66. In some cases, the antibody provided herein comprises a heavy chain variable region comprising an amino acid sequence having at least 99% sequence identity to the amino acid sequence as set forth SEQ ID NO: 45, and a light chain variable region comprising an amino acid sequence having at least 99% sequence identity to the amino acid sequence as set forth SEQ ID NO: 66. In some cases, the antibody provided herein comprises a heavy chain variable region comprising an amino acid sequence having 100% sequence identity to the amino acid sequence as set forth SEQ ID NO: 45, and a light chain variable region comprising an amino acid sequence having 100% sequence identity to the amino acid sequence as set forth SEQ ID NO: 66. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 45, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 66. In some cases, the antibody provided herein comprises a heavy chain variable region comprising an amino acid sequence having less than or equal to 10 total substitutions, deletions, and/or insertions in the amino acid sequence as set forth SEQ ID NO: 45, and a light chain variable region comprising an amino acid sequence having less than or equal to 10 total substitutions, deletions, and/or insertions in the amino acid sequence as set forth SEQ ID NO: 66. In some cases, the antibody provided herein comprises a heavy chain variable region comprising an amino acid sequence having less than or equal to 7 total substitutions, deletions, and/or insertions in the amino acid sequence as set forth SEQ ID NO: 45, and a light chain variable region comprising an amino acid sequence having less than or equal to 7 total substitutions, deletions, and/or insertions in the amino acid sequence as set forth SEQ ID NO: 66. In some cases, the antibody provided herein comprises a heavy chain variable region comprising an amino acid sequence having less than or equal to 5 total substitutions, deletions, and/or insertions in the amino acid sequence as set forth SEQ ID NO: 45, and a light chain variable region comprising an amino acid sequence having less than or equal to 5 total substitutions, deletions, and/or insertions in the amino acid sequence as set forth SEQ ID NO: 66. In some cases, the antibody provided herein comprises a heavy chain variable region comprising an amino acid sequence having less than or equal to 3 total substitutions, deletions, and/or insertions in the amino acid sequence as set forth SEQ ID NO: 45, and a light chain variable region comprising an amino acid sequence having less than or equal to 3 total substitutions, deletions, and/or insertions in the amino acid sequence as set forth SEQ ID NO: 66. In some cases, the antibody provided herein comprises a heavy chain variable region comprising an amino acid sequence having less than or equal to 2 total substitutions, deletions, and/or insertions in the amino acid sequence as set forth SEQ ID NO: 45, and a light chain variable region comprising an amino acid sequence having less than or equal to 2 total substitutions, deletions, and/or insertions in the amino acid sequence as set forth SEQ ID NO: 66. In some cases, the antibody provided herein comprises a heavy chain variable region comprising an amino acid sequence having less than or equal to 1 total substitutions, deletions, and/or insertions in the amino acid sequence as set forth SEQ ID NO: 45, and a light chain variable region comprising an amino acid sequence having less than or equal to 1 total substitutions, deletions, and/or insertions in the amino acid sequence as set forth SEQ ID NO: 66.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 46, and a light chain variable region comprising an amino acid sequence having at least 90%, 95%, 96%, 97%, 98%, 99%, or 100% sequence identity to the amino acid sequence as set forth SEQ ID NO: 2. In some cases, the antibody provided herein comprises a heavy chain variable region comprising an amino acid sequence having at least 95% sequence identity to the amino acid sequence as set forth SEQ ID NO: 46, and a light chain variable region comprising an amino acid sequence having at least 95% sequence identity to the amino acid sequence as set forth SEQ ID NO: 2. In some cases, the antibody provided herein comprises a heavy chain variable region comprising an amino acid sequence having at least 97% sequence identity to the amino acid sequence as set forth SEQ ID NO: 46, and a light chain variable region comprising an amino acid sequence having at least 97% sequence identity to the amino acid sequence as set forth SEQ ID NO: 2. In some cases, the antibody provided herein comprises a heavy chain variable region comprising an amino acid sequence having at least 99% sequence identity to the amino acid sequence as set forth SEQ ID NO: 46, and a light chain variable region comprising an amino acid sequence having at least 99% sequence identity to the amino acid sequence as set forth SEQ ID NO: 2. In some cases, the antibody provided herein comprises a heavy chain variable region comprising an amino acid sequence having 100% sequence identity to the amino acid sequence as set forth SEQ ID NO: 46, and a light chain variable region comprising an amino acid sequence having 100% sequence identity to the amino acid sequence as set forth SEQ ID NO: 2. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 46, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 2. In some cases, the antibody provided herein comprises a heavy chain variable region comprising an amino acid sequence having less than or equal to 10 total substitutions, deletions, and/or insertions in the amino acid sequence as set forth SEQ ID NO: 46, and a light chain variable region comprising an amino acid sequence having less than or equal to 10 total substitutions, deletions, and/or insertions in the amino acid sequence as set forth SEQ ID NO: 2. In some cases, the antibody provided herein comprises a heavy chain variable region comprising an amino acid sequence having less than or equal to 7 total substitutions, deletions, and/or insertions in the amino acid sequence as set forth SEQ ID NO: 46, and a light chain variable region comprising an amino acid sequence having less than or equal to 7 total substitutions, deletions, and/or insertions in the amino acid sequence as set forth SEQ ID NO: 2. In some cases, the antibody provided herein comprises a heavy chain variable region comprising an amino acid sequence having less than or equal to 5 total substitutions, deletions, and/or insertions in the amino acid sequence as set forth SEQ ID NO: 46, and a light chain variable region comprising an amino acid sequence having less than or equal to 5 total substitutions, deletions, and/or insertions in the amino acid sequence as set forth SEQ ID NO: 2. In some cases, the antibody provided herein comprises a heavy chain variable region comprising an amino acid sequence having less than or equal to 3 total substitutions, deletions, and/or insertions in the amino acid sequence as set forth SEQ ID NO: 46, and a light chain variable region comprising an amino acid sequence having less than or equal to 3 total substitutions, deletions, and/or insertions in the amino acid sequence as set forth SEQ ID NO: 2. In some cases, the antibody provided herein comprises a heavy chain variable region comprising an amino acid sequence having less than or equal to 2 total substitutions, deletions, and/or insertions in the amino acid sequence as set forth SEQ ID NO: 46, and a light chain variable region comprising an amino acid sequence having less than or equal to 2 total substitutions, deletions, and/or insertions in the amino acid sequence as set forth SEQ ID NO: 2. In some cases, the antibody provided herein comprises a heavy chain variable region comprising an amino acid sequence having less than or equal to 1 total substitutions, deletions, and/or insertions in the amino acid sequence as set forth SEQ ID NO: 46, and a light chain variable region comprising an amino acid sequence having less than or equal to 1 total substitutions, deletions, and/or insertions in the amino acid sequence as set forth SEQ ID NO: 2.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 46, and a light chain variable region comprising an amino acid sequence having at least 90%, 95%, 96%, 97%, 98%, 99%, or 100% sequence identity to the amino acid sequence as set forth SEQ ID NO: 48. In some cases, the antibody provided herein comprises a heavy chain variable region comprising an amino acid sequence having at least 95% sequence identity to the amino acid sequence as set forth SEQ ID NO: 46, and a light chain variable region comprising an amino acid sequence having at least 95% sequence identity to the amino acid sequence as set forth SEQ ID NO: 48. In some cases, the antibody provided herein comprises a heavy chain variable region comprising an amino acid sequence having at least 97% sequence identity to the amino acid sequence as set forth SEQ ID NO: 46, and a light chain variable region comprising an amino acid sequence having at least 97% sequence identity to the amino acid sequence as set forth SEQ ID NO: 48. In some cases, the antibody provided herein comprises a heavy chain variable region comprising an amino acid sequence having at least 99% sequence identity to the amino acid sequence as set forth SEQ ID NO: 46, and a light chain variable region comprising an amino acid sequence having at least 99% sequence identity to the amino acid sequence as set forth SEQ ID NO: 48. In some cases, the antibody provided herein comprises a heavy chain variable region comprising an amino acid sequence having 100% sequence identity to the amino acid sequence as set forth SEQ ID NO: 46, and a light chain variable region comprising an amino acid sequence having 100% sequence identity to the amino acid sequence as set forth SEQ ID NO: 48. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 46, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 48. In some cases, the antibody provided herein comprises a heavy chain variable region comprising an amino acid sequence having less than or equal to 10 total substitutions, deletions, and/or insertions in the amino acid sequence as set forth SEQ ID NO: 46, and a light chain variable region comprising an amino acid sequence having less than or equal to 10 total substitutions, deletions, and/or insertions in the amino acid sequence as set forth SEQ ID NO: 48. In some cases, the antibody provided herein comprises a heavy chain variable region comprising an amino acid sequence having less than or equal to 7 total substitutions, deletions, and/or insertions in the amino acid sequence as set forth SEQ ID NO: 46, and a light chain variable region comprising an amino acid sequence having less than or equal to 7 total substitutions, deletions, and/or insertions in the amino acid sequence as set forth SEQ ID NO: 48. In some cases, the antibody provided herein comprises a heavy chain variable region comprising an amino acid sequence having less than or equal to 5 total substitutions, deletions, and/or insertions in the amino acid sequence as set forth SEQ ID NO: 46, and a light chain variable region comprising an amino acid sequence having less than or equal to 5 total substitutions, deletions, and/or insertions in the amino acid sequence as set forth SEQ ID NO: 48. In some cases, the antibody provided herein comprises a heavy chain variable region comprising an amino acid sequence having less than or equal to 3 total substitutions, deletions, and/or insertions in the amino acid sequence as set forth SEQ ID NO: 46, and a light chain variable region comprising an amino acid sequence having less than or equal to 3 total substitutions, deletions, and/or insertions in the amino acid sequence as set forth SEQ ID NO: 48. In some cases, the antibody provided herein comprises a heavy chain variable region comprising an amino acid sequence having less than or equal to 2 total substitutions, deletions, and/or insertions in the amino acid sequence as set forth SEQ ID NO: 46, and a light chain variable region comprising an amino acid sequence having less than or equal to 2 total substitutions, deletions, and/or insertions in the amino acid sequence as set forth SEQ ID NO: 48. In some cases, the antibody provided herein comprises a heavy chain variable region comprising an amino acid sequence having less than or equal to 1 total substitutions, deletions, and/or insertions in the amino acid sequence as set forth SEQ ID NO: 46, and a light chain variable region comprising an amino acid sequence having less than or equal to 1 total substitutions, deletions, and/or insertions in the amino acid sequence as set forth SEQ ID NO: 48

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 46, and a light chain variable region comprising an amino acid sequence having at least 90%, 95%, 96%, 97%, 98%, 99%, or 100% sequence identity to the amino acid sequence as set forth SEQ ID NO: 49. In some cases, the antibody provided herein comprises a heavy chain variable region comprising an amino acid sequence having at least 95% sequence identity to the amino acid sequence as set forth SEQ ID NO: 46, and a light chain variable region comprising an amino acid sequence having at least 95% sequence identity to the amino acid sequence as set forth SEQ ID NO: 49. In some cases, the antibody provided herein comprises a heavy chain variable region comprising an amino acid sequence having at least 97% sequence identity to the amino acid sequence as set forth SEQ ID NO: 46, and a light chain variable region comprising an amino acid sequence having at least 97% sequence identity to the amino acid sequence as set forth SEQ ID NO: 49. In some cases, the antibody provided herein comprises a heavy chain variable region comprising an amino acid sequence having at least 99% sequence identity to the amino acid sequence as set forth SEQ ID NO: 46, and a light chain variable region comprising an amino acid sequence having at least 99% sequence identity to the amino acid sequence as set forth SEQ ID NO: 49. In some cases, the antibody provided herein comprises a heavy chain variable region comprising an amino acid sequence having 100% sequence identity to the amino acid sequence as set forth SEQ ID NO: 46, and a light chain variable region comprising an amino acid sequence having 100% sequence identity to the amino acid sequence as set forth SEQ ID NO: 49. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 46, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 49. In some cases, the antibody provided herein comprises a heavy chain variable region comprising an amino acid sequence having less than or equal to 10 total substitutions, deletions, and/or insertions in the amino acid sequence as set forth SEQ ID NO: 46, and a light chain variable region comprising an amino acid sequence having less than or equal to 10 total substitutions, deletions, and/or insertions in the amino acid sequence as set forth SEQ ID NO: 49. In some cases, the antibody provided herein comprises a heavy chain variable region comprising an amino acid sequence having less than or equal to 7 total substitutions, deletions, and/or insertions in the amino acid sequence as set forth SEQ ID NO: 46, and a light chain variable region comprising an amino acid sequence having less than or equal to 7 total substitutions, deletions, and/or insertions in the amino acid sequence as set forth SEQ ID NO: 49. In some cases, the antibody provided herein comprises a heavy chain variable region comprising an amino acid sequence having less than or equal to 5 total substitutions, deletions, and/or insertions in the amino acid sequence as set forth SEQ ID NO: 46, and a light chain variable region comprising an amino acid sequence having less than or equal to 5 total substitutions, deletions, and/or insertions in the amino acid sequence as set forth SEQ ID NO: 49. In some cases, the antibody provided herein comprises a heavy chain variable region comprising an amino acid sequence having less than or equal to 3 total substitutions, deletions, and/or insertions in the amino acid sequence as set forth SEQ ID NO: 46, and a light chain variable region comprising an amino acid sequence having less than or equal to 3 total substitutions, deletions, and/or insertions in the amino acid sequence as set forth SEQ ID NO: 49. In some cases, the antibody provided herein comprises a heavy chain variable region comprising an amino acid sequence having less than or equal to 2 total substitutions, deletions, and/or insertions in the amino acid sequence as set forth SEQ ID NO: 46, and a light chain variable region comprising an amino acid sequence having less than or equal to 2 total substitutions, deletions, and/or insertions in the amino acid sequence as set forth SEQ ID NO: 49. In some cases, the antibody provided herein comprises a heavy chain variable region comprising an amino acid sequence having less than or equal to 1 total substitutions, deletions, and/or insertions in the amino acid sequence as set forth SEQ ID NO: 46, and a light chain variable region comprising an amino acid sequence having less than or equal to 1 total substitutions, deletions, and/or insertions in the amino acid sequence as set forth SEQ ID NO: 49.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 46, and a light chain variable region comprising an amino acid sequence having at least 90%, 95%, 96%, 97%, 98%, 99%, or 100% sequence identity to the amino acid sequence as set forth SEQ ID NO: 50. In some cases, the antibody provided herein comprises a heavy chain variable region comprising an amino acid sequence having at least 95% sequence identity to the amino acid sequence as set forth SEQ ID NO: 46, and a light chain variable region comprising an amino acid sequence having at least 95% sequence identity to the amino acid sequence as set forth SEQ ID NO: 50. In some cases, the antibody provided herein comprises a heavy chain variable region comprising an amino acid sequence having at least 97% sequence identity to the amino acid sequence as set forth SEQ ID NO: 46, and a light chain variable region comprising an amino acid sequence having at least 97% sequence identity to the amino acid sequence as set forth SEQ ID NO: 50. In some cases, the antibody provided herein comprises a heavy chain variable region comprising an amino acid sequence having at least 99% sequence identity to the amino acid sequence as set forth SEQ ID NO: 46, and a light chain variable region comprising an amino acid sequence having at least 99% sequence identity to the amino acid sequence as set forth SEQ ID NO: 50. In some cases, the antibody provided herein comprises a heavy chain variable region comprising an amino acid sequence having 100% sequence identity to the amino acid sequence as set forth SEQ ID NO: 46, and a light chain variable region comprising an amino acid sequence having 100% sequence identity to the amino acid sequence as set forth SEQ ID NO: 50. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 46, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 50. In some cases, the antibody provided herein comprises a heavy chain variable region comprising an amino acid sequence having less than or equal to 10 total substitutions, deletions, and/or insertions in the amino acid sequence as set forth SEQ ID NO: 46, and a light chain variable region comprising an amino acid sequence having less than or equal to 10 total substitutions, deletions, and/or insertions in the amino acid sequence as set forth SEQ ID NO: 50. In some cases, the antibody provided herein comprises a heavy chain variable region comprising an amino acid sequence having less than or equal to 7 total substitutions, deletions, and/or insertions in the amino acid sequence as set forth SEQ ID NO: 46, and a light chain variable region comprising an amino acid sequence having less than or equal to 7 total substitutions, deletions, and/or insertions in the amino acid sequence as set forth SEQ ID NO: 50. In some cases, the antibody provided herein comprises a heavy chain variable region comprising an amino acid sequence having less than or equal to 5 total substitutions, deletions, and/or insertions in the amino acid sequence as set forth SEQ ID NO: 46, and a light chain variable region comprising an amino acid sequence having less than or equal to 5 total substitutions, deletions, and/or insertions in the amino acid sequence as set forth SEQ ID NO: 50. In some cases, the antibody provided herein comprises a heavy chain variable region comprising an amino acid sequence having less than or equal to 3 total substitutions, deletions, and/or insertions in the amino acid sequence as set forth SEQ ID NO: 46, and a light chain variable region comprising an amino acid sequence having less than or equal to 3 total substitutions, deletions, and/or insertions in the amino acid sequence as set forth SEQ ID NO: 50. In some cases, the antibody provided herein comprises a heavy chain variable region comprising an amino acid sequence having less than or equal to 2 total substitutions, deletions, and/or insertions in the amino acid sequence as set forth SEQ ID NO: 46, and a light chain variable region comprising an amino acid sequence having less than or equal to 2 total substitutions, deletions, and/or insertions in the amino acid sequence as set forth SEQ ID NO: 50. In some cases, the antibody provided herein comprises a heavy chain variable region comprising an amino acid sequence having less than or equal to 1 total substitutions, deletions, and/or insertions in the amino acid sequence as set forth SEQ ID NO: 46, and a light chain variable region comprising an amino acid sequence having less than or equal to 1 total substitutions, deletions, and/or insertions in the amino acid sequence as set forth SEQ ID NO: 50.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 46, and a light chain variable region comprising an amino acid sequence having at least 90%, 95%, 96%, 97%, 98%, 99%, or 100% sequence identity to the amino acid sequence as set forth SEQ ID NO: 51. In some cases, the antibody provided herein comprises a heavy chain variable region comprising an amino acid sequence having at least 95% sequence identity to the amino acid sequence as set forth SEQ ID NO: 46, and a light chain variable region comprising an amino acid sequence having at least 95% sequence identity to the amino acid sequence as set forth SEQ ID NO: 51. In some cases, the antibody provided herein comprises a heavy chain variable region comprising an amino acid sequence having at least 97% sequence identity to the amino acid sequence as set forth SEQ ID NO: 46, and a light chain variable region comprising an amino acid sequence having at least 97% sequence identity to the amino acid sequence as set forth SEQ ID NO: 51. In some cases, the antibody provided herein comprises a heavy chain variable region comprising an amino acid sequence having at least 99% sequence identity to the amino acid sequence as set forth SEQ ID NO: 46, and a light chain variable region comprising an amino acid sequence having at least 99% sequence identity to the amino acid sequence as set forth SEQ ID NO: 51. In some cases, the antibody provided herein comprises a heavy chain variable region comprising an amino acid sequence having 100% sequence identity to the amino acid sequence as set forth SEQ ID NO: 46, and a light chain variable region comprising an amino acid sequence having 100% sequence identity to the amino acid sequence as set forth SEQ ID NO: 51. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 46, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 51. In some cases, the antibody provided herein comprises a heavy chain variable region comprising an amino acid sequence having less than or equal to 10 total substitutions, deletions, and/or insertions in the amino acid sequence as set forth SEQ ID NO: 46, and a light chain variable region comprising an amino acid sequence having less than or equal to 10 total substitutions, deletions, and/or insertions in the amino acid sequence as set forth SEQ ID NO: 51. In some cases, the antibody provided herein comprises a heavy chain variable region comprising an amino acid sequence having less than or equal to 7 total substitutions, deletions, and/or insertions in the amino acid sequence as set forth SEQ ID NO: 46, and a light chain variable region comprising an amino acid sequence having less than or equal to 7 total substitutions, deletions, and/or insertions in the amino acid sequence as set forth SEQ ID NO: 51. In some cases, the antibody provided herein comprises a heavy chain variable region comprising an amino acid sequence having less than or equal to 5 total substitutions, deletions, and/or insertions in the amino acid sequence as set forth SEQ ID NO: 46, and a light chain variable region comprising an amino acid sequence having less than or equal to 5 total substitutions, deletions, and/or insertions in the amino acid sequence as set forth SEQ ID NO: 51. In some cases, the antibody provided herein comprises a heavy chain variable region comprising an amino acid sequence having less than or equal to 3 total substitutions, deletions, and/or insertions in the amino acid sequence as set forth SEQ ID NO: 46, and a light chain variable region comprising an amino acid sequence having less than or equal to 3 total substitutions, deletions, and/or insertions in the amino acid sequence as set forth SEQ ID NO: 51. In some cases, the antibody provided herein comprises a heavy chain variable region comprising an amino acid sequence having less than or equal to 2 total substitutions, deletions, and/or insertions in the amino acid sequence as set forth SEQ ID NO: 46, and a light chain variable region comprising an amino acid sequence having less than or equal to 2 total substitutions, deletions, and/or insertions in the amino acid sequence as set forth SEQ ID NO: 51. In some cases, the antibody provided herein comprises a heavy chain variable region comprising an amino acid sequence having less than or equal to 1 total substitutions, deletions, and/or insertions in the amino acid sequence as set forth SEQ ID NO: 46, and a light chain variable region comprising an amino acid sequence having less than or equal to 1 total substitutions, deletions, and/or insertions in the amino acid sequence as set forth SEQ ID NO: 51

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 46, and a light chain variable region comprising an amino acid sequence having at least 90%, 95%, 96%, 97%, 98%, 99%, or 100% sequence identity to the amino acid sequence as set forth SEQ ID NO: 52. In some cases, the antibody provided herein comprises a heavy chain variable region comprising an amino acid sequence having at least 95% sequence identity to the amino acid sequence as set forth SEQ ID NO: 46, and a light chain variable region comprising an amino acid sequence having at least 95% sequence identity to the amino acid sequence as set forth SEQ ID NO: 52. In some cases, the antibody provided herein comprises a heavy chain variable region comprising an amino acid sequence having at least 97% sequence identity to the amino acid sequence as set forth SEQ ID NO: 46, and a light chain variable region comprising an amino acid sequence having at least 97% sequence identity to the amino acid sequence as set forth SEQ ID NO: 52. In some cases, the antibody provided herein comprises a heavy chain variable region comprising an amino acid sequence having at least 99% sequence identity to the amino acid sequence as set forth SEQ ID NO: 46, and a light chain variable region comprising an amino acid sequence having at least 99% sequence identity to the amino acid sequence as set forth SEQ ID NO: 52. In some cases, the antibody provided herein comprises a heavy chain variable region comprising an amino acid sequence having 100% sequence identity to the amino acid sequence as set forth SEQ ID NO: 46, and a light chain variable region comprising an amino acid sequence having 100% sequence identity to the amino acid sequence as set forth SEQ ID NO: 52. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 46, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 52. In some cases, the antibody provided herein comprises a heavy chain variable region comprising an amino acid sequence having less than or equal to 10 total substitutions, deletions, and/or insertions in the amino acid sequence as set forth SEQ ID NO: 46, and a light chain variable region comprising an amino acid sequence having less than or equal to 10 total substitutions, deletions, and/or insertions in the amino acid sequence as set forth SEQ ID NO: 52. In some cases, the antibody provided herein comprises a heavy chain variable region comprising an amino acid sequence having less than or equal to 7 total substitutions, deletions, and/or insertions in the amino acid sequence as set forth SEQ ID NO: 46, and a light chain variable region comprising an amino acid sequence having less than or equal to 7 total substitutions, deletions, and/or insertions in the amino acid sequence as set forth SEQ ID NO: 52. In some cases, the antibody provided herein comprises a heavy chain variable region comprising an amino acid sequence having less than or equal to 5 total substitutions, deletions, and/or insertions in the amino acid sequence as set forth SEQ ID NO: 46, and a light chain variable region comprising an amino acid sequence having less than or equal to 5 total substitutions, deletions, and/or insertions in the amino acid sequence as set forth SEQ ID NO: 52. In some cases, the antibody provided herein comprises a heavy chain variable region comprising an amino acid sequence having less than or equal to 3 total substitutions, deletions, and/or insertions in the amino acid sequence as set forth SEQ ID NO: 46, and a light chain variable region comprising an amino acid sequence having less than or equal to 3 total substitutions, deletions, and/or insertions in the amino acid sequence as set forth SEQ ID NO: 52. In some cases, the antibody provided herein comprises a heavy chain variable region comprising an amino acid sequence having less than or equal to 2 total substitutions, deletions, and/or insertions in the amino acid sequence as set forth SEQ ID NO: 46, and a light chain variable region comprising an amino acid sequence having less than or equal to 2 total substitutions, deletions, and/or insertions in the amino acid sequence as set forth SEQ ID NO: 52. In some cases, the antibody provided herein comprises a heavy chain variable region comprising an amino acid sequence having less than or equal to 1 total substitutions, deletions, and/or insertions in the amino acid sequence as set forth SEQ ID NO: 46, and a light chain variable region comprising an amino acid sequence having less than or equal to 1 total substitutions, deletions, and/or insertions in the amino acid sequence as set forth SEQ ID NO: 52.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 46, and a light chain variable region comprising an amino acid sequence having at least 90%, 95%, 96%, 97%, 98%, 99%, or 100% sequence identity to the amino acid sequence as set forth SEQ ID NO: 65. In some cases, the antibody provided herein comprises a heavy chain variable region comprising an amino acid sequence having at least 95% sequence identity to the amino acid sequence as set forth SEQ ID NO: 46, and a light chain variable region comprising an amino acid sequence having at least 95% sequence identity to the amino acid sequence as set forth SEQ ID NO: 65. In some cases, the antibody provided herein comprises a heavy chain variable region comprising an amino acid sequence having at least 97% sequence identity to the amino acid sequence as set forth SEQ ID NO: 46, and a light chain variable region comprising an amino acid sequence having at least 97% sequence identity to the amino acid sequence as set forth SEQ ID NO: 65. In some cases, the antibody provided herein comprises a heavy chain variable region comprising an amino acid sequence having at least 99% sequence identity to the amino acid sequence as set forth SEQ ID NO: 46, and a light chain variable region comprising an amino acid sequence having at least 99% sequence identity to the amino acid sequence as set forth SEQ ID NO: 65. In some cases, the antibody provided herein comprises a heavy chain variable region comprising an amino acid sequence having 100% sequence identity to the amino acid sequence as set forth SEQ ID NO: 46, and a light chain variable region comprising an amino acid sequence having 100% sequence identity to the amino acid sequence as set forth SEQ ID NO: 65. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 46, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 65. In some cases, the antibody provided herein comprises a heavy chain variable region comprising an amino acid sequence having less than or equal to 10 total substitutions, deletions, and/or insertions in the amino acid sequence as set forth SEQ ID NO: 46, and a light chain variable region comprising an amino acid sequence having less than or equal to 10 total substitutions, deletions, and/or insertions in the amino acid sequence as set forth SEQ ID NO: 65. In some cases, the antibody provided herein comprises a heavy chain variable region comprising an amino acid sequence having less than or equal to 7 total substitutions, deletions, and/or insertions in the amino acid sequence as set forth SEQ ID NO: 46, and a light chain variable region comprising an amino acid sequence having less than or equal to 7 total substitutions, deletions, and/or insertions in the amino acid sequence as set forth SEQ ID NO: 65. In some cases, the antibody provided herein comprises a heavy chain variable region comprising an amino acid sequence having less than or equal to 5 total substitutions, deletions, and/or insertions in the amino acid sequence as set forth SEQ ID NO: 46, and a light chain variable region comprising an amino acid sequence having less than or equal to 5 total substitutions, deletions, and/or insertions in the amino acid sequence as set forth SEQ ID NO: 65. In some cases, the antibody provided herein comprises a heavy chain variable region comprising an amino acid sequence having less than or equal to 3 total substitutions, deletions, and/or insertions in the amino acid sequence as set forth SEQ ID NO: 46, and a light chain variable region comprising an amino acid sequence having less than or equal to 3 total substitutions, deletions, and/or insertions in the amino acid sequence as set forth SEQ ID NO: 65. In some cases, the antibody provided herein comprises a heavy chain variable region comprising an amino acid sequence having less than or equal to 2 total substitutions, deletions, and/or insertions in the amino acid sequence as set forth SEQ ID NO: 46, and a light chain variable region comprising an amino acid sequence having less than or equal to 2 total substitutions, deletions, and/or insertions in the amino acid sequence as set forth SEQ ID NO: 65. In some cases, the antibody provided herein comprises a heavy chain variable region comprising an amino acid sequence having less than or equal to 1 total substitutions, deletions, and/or insertions in the amino acid sequence as set forth SEQ ID NO: 46, and a light chain variable region comprising an amino acid sequence having less than or equal to 1 total substitutions, deletions, and/or insertions in the amino acid sequence as set forth SEQ ID NO: 65.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 46, and a light chain variable region comprising an amino acid sequence having at least 90%, 95%, 96%, 97%, 98%, 99%, or 100% sequence identity to the amino acid sequence as set forth SEQ ID NO: 66. In some cases, the antibody provided herein comprises a heavy chain variable region comprising an amino acid sequence having at least 95% sequence identity to the amino acid sequence as set forth SEQ ID NO: 46, and a light chain variable region comprising an amino acid sequence having at least 95% sequence identity to the amino acid sequence as set forth SEQ ID NO: 66. In some cases, the antibody provided herein comprises a heavy chain variable region comprising an amino acid sequence having at least 97% sequence identity to the amino acid sequence as set forth SEQ ID NO: 46, and a light chain variable region comprising an amino acid sequence having at least 97% sequence identity to the amino acid sequence as set forth SEQ ID NO: 66. In some cases, the antibody provided herein comprises a heavy chain variable region comprising an amino acid sequence having at least 99% sequence identity to the amino acid sequence as set forth SEQ ID NO: 46, and a light chain variable region comprising an amino acid sequence having at least 99% sequence identity to the amino acid sequence as set forth SEQ ID NO: 66. In some cases, the antibody provided herein comprises a heavy chain variable region comprising an amino acid sequence having 100% sequence identity to the amino acid sequence as set forth SEQ ID NO: 46, and a light chain variable region comprising an amino acid sequence having 100% sequence identity to the amino acid sequence as set forth SEQ ID NO: 66. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 46, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 66. In some cases, the antibody provided herein comprises a heavy chain variable region comprising an amino acid sequence having less than or equal to 10 total substitutions, deletions, and/or insertions in the amino acid sequence as set forth SEQ ID NO: 46, and a light chain variable region comprising an amino acid sequence having less than or equal to 10 total substitutions, deletions, and/or insertions in the amino acid sequence as set forth SEQ ID NO: 66. In some cases, the antibody provided herein comprises a heavy chain variable region comprising an amino acid sequence having less than or equal to 7 total substitutions, deletions, and/or insertions in the amino acid sequence as set forth SEQ ID NO: 46, and a light chain variable region comprising an amino acid sequence having less than or equal to 7 total substitutions, deletions, and/or insertions in the amino acid sequence as set forth SEQ ID NO: 66. In some cases, the antibody provided herein comprises a heavy chain variable region comprising an amino acid sequence having less than or equal to 5 total substitutions, deletions, and/or insertions in the amino acid sequence as set forth SEQ ID NO: 46, and a light chain variable region comprising an amino acid sequence having less than or equal to 5 total substitutions, deletions, and/or insertions in the amino acid sequence as set forth SEQ ID NO: 66. In some cases, the antibody provided herein comprises a heavy chain variable region comprising an amino acid sequence having less than or equal to 3 total substitutions, deletions, and/or insertions in the amino acid sequence as set forth SEQ ID NO: 46, and a light chain variable region comprising an amino acid sequence having less than or equal to 3 total substitutions, deletions, and/or insertions in the amino acid sequence as set forth SEQ ID NO: 66. In some cases, the antibody provided herein comprises a heavy chain variable region comprising an amino acid sequence having less than or equal to 2 total substitutions, deletions, and/or insertions in the amino acid sequence as set forth SEQ ID NO: 46, and a light chain variable region comprising an amino acid sequence having less than or equal to 2 total substitutions, deletions, and/or insertions in the amino acid sequence as set forth SEQ ID NO: 66. In some cases, the antibody provided herein comprises a heavy chain variable region comprising an amino acid sequence having less than or equal to 1 total substitutions, deletions, and/or insertions in the amino acid sequence as set forth SEQ ID NO: 46, and a light chain variable region comprising an amino acid sequence having less than or equal to 1 total substitutions, deletions, and/or insertions in the amino acid sequence as set forth SEQ ID NO: 66.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 47, and a light chain variable region comprising an amino acid sequence having at least 90%, 95%, 96%, 97%, 98%, 99%, or 100% sequence identity to the amino acid sequence as set forth SEQ ID NO: 2. In some cases, the antibody provided herein comprises a heavy chain variable region comprising an amino acid sequence having at least 95% sequence identity to the amino acid sequence as set forth SEQ ID NO: 47, and a light chain variable region comprising an amino acid sequence having at least 95% sequence identity to the amino acid sequence as set forth SEQ ID NO: 2. In some cases, the antibody provided herein comprises a heavy chain variable region comprising an amino acid sequence having at least 97% sequence identity to the amino acid sequence as set forth SEQ ID NO: 47, and a light chain variable region comprising an amino acid sequence having at least 97% sequence identity to the amino acid sequence as set forth SEQ ID NO: 2. In some cases, the antibody provided herein comprises a heavy chain variable region comprising an amino acid sequence having at least 99% sequence identity to the amino acid sequence as set forth SEQ ID NO: 47, and a light chain variable region comprising an amino acid sequence having at least 99% sequence identity to the amino acid sequence as set forth SEQ ID NO: 2. In some cases, the antibody provided herein comprises a heavy chain variable region comprising an amino acid sequence having 100% sequence identity to the amino acid sequence as set forth SEQ ID NO: 47, and a light chain variable region comprising an amino acid sequence having 100% sequence identity to the amino acid sequence as set forth SEQ ID NO: 2. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 47, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 2. In some cases, the antibody provided herein comprises a heavy chain variable region comprising an amino acid sequence having less than or equal to 10 total substitutions, deletions, and/or insertions in the amino acid sequence as set forth SEQ ID NO: 47, and a light chain variable region comprising an amino acid sequence having less than or equal to 10 total substitutions, deletions, and/or insertions in the amino acid sequence as set forth SEQ ID NO: 2. In some cases, the antibody provided herein comprises a heavy chain variable region comprising an amino acid sequence having less than or equal to 7 total substitutions, deletions, and/or insertions in the amino acid sequence as set forth SEQ ID NO: 47, and a light chain variable region comprising an amino acid sequence having less than or equal to 7 total substitutions, deletions, and/or insertions in the amino acid sequence as set forth SEQ ID NO: 2. In some cases, the antibody provided herein comprises a heavy chain variable region comprising an amino acid sequence having less than or equal to 5 total substitutions, deletions, and/or insertions in the amino acid sequence as set forth SEQ ID NO: 47, and a light chain variable region comprising an amino acid sequence having less than or equal to 5 total substitutions, deletions, and/or insertions in the amino acid sequence as set forth SEQ ID NO: 2. In some cases, the antibody provided herein comprises a heavy chain variable region comprising an amino acid sequence having less than or equal to 3 total substitutions, deletions, and/or insertions in the amino acid sequence as set forth SEQ ID NO: 47, and a light chain variable region comprising an amino acid sequence having less than or equal to 3 total substitutions, deletions, and/or insertions in the amino acid sequence as set forth SEQ ID NO: 2. In some cases, the antibody provided herein comprises a heavy chain variable region comprising an amino acid sequence having less than or equal to 2 total substitutions, deletions, and/or insertions in the amino acid sequence as set forth SEQ ID NO: 47, and a light chain variable region comprising an amino acid sequence having less than or equal to 2 total substitutions, deletions, and/or insertions in the amino acid sequence as set forth SEQ ID NO: 2. In some cases, the antibody provided herein comprises a heavy chain variable region comprising an amino acid sequence having less than or equal to 1 total substitutions, deletions, and/or insertions in the amino acid sequence as set forth SEQ ID NO: 47, and a light chain variable region comprising an amino acid sequence having less than or equal to 1 total substitutions, deletions, and/or insertions in the amino acid sequence as set forth SEQ ID NO: 2.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 47, and a light chain variable region comprising an amino acid sequence having at least 90%, 95%, 96%, 97%, 98%, 99%, or 100% sequence identity to the amino acid sequence as set forth SEQ ID NO: 48. In some cases, the antibody provided herein comprises a heavy chain variable region comprising an amino acid sequence having at least 95% sequence identity to the amino acid sequence as set forth SEQ ID NO: 47, and a light chain variable region comprising an amino acid sequence having at least 95% sequence identity to the amino acid sequence as set forth SEQ ID NO: 48. In some cases, the antibody provided herein comprises a heavy chain variable region comprising an amino acid sequence having at least 97% sequence identity to the amino acid sequence as set forth SEQ ID NO: 47, and a light chain variable region comprising an amino acid sequence having at least 97% sequence identity to the amino acid sequence as set forth SEQ ID NO: 48. In some cases, the antibody provided herein comprises a heavy chain variable region comprising an amino acid sequence having at least 99% sequence identity to the amino acid sequence as set forth SEQ ID NO: 47, and a light chain variable region comprising an amino acid sequence having at least 99% sequence identity to the amino acid sequence as set forth SEQ ID NO: 48. In some cases, the antibody provided herein comprises a heavy chain variable region comprising an amino acid sequence having 100% sequence identity to the amino acid sequence as set forth SEQ ID NO: 47, and a light chain variable region comprising an amino acid sequence having 100% sequence identity to the amino acid sequence as set forth SEQ ID NO: 48. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 47, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 48. In some cases, the antibody provided herein comprises a heavy chain variable region comprising an amino acid sequence having less than or equal to 10 total substitutions, deletions, and/or insertions in the amino acid sequence as set forth SEQ ID NO: 47, and a light chain variable region comprising an amino acid sequence having less than or equal to 10 total substitutions, deletions, and/or insertions in the amino acid sequence as set forth SEQ ID NO: 48. In some cases, the antibody provided herein comprises a heavy chain variable region comprising an amino acid sequence having less than or equal to 7 total substitutions, deletions, and/or insertions in the amino acid sequence as set forth SEQ ID NO: 47, and a light chain variable region comprising an amino acid sequence having less than or equal to 7 total substitutions, deletions, and/or insertions in the amino acid sequence as set forth SEQ ID NO: 48. In some cases, the antibody provided herein comprises a heavy chain variable region comprising an amino acid sequence having less than or equal to 5 total substitutions, deletions, and/or insertions in the amino acid sequence as set forth SEQ ID NO: 47, and a light chain variable region comprising an amino acid sequence having less than or equal to 5 total substitutions, deletions, and/or insertions in the amino acid sequence as set forth SEQ ID NO: 48. In some cases, the antibody provided herein comprises a heavy chain variable region comprising an amino acid sequence having less than or equal to 3 total substitutions, deletions, and/or insertions in the amino acid sequence as set forth SEQ ID NO: 47, and a light chain variable region comprising an amino acid sequence having less than or equal to 3 total substitutions, deletions, and/or insertions in the amino acid sequence as set forth SEQ ID NO: 48. In some cases, the antibody provided herein comprises a heavy chain variable region comprising an amino acid sequence having less than or equal to 2 total substitutions, deletions, and/or insertions in the amino acid sequence as set forth SEQ ID NO: 47, and a light chain variable region comprising an amino acid sequence having less than or equal to 2 total substitutions, deletions, and/or insertions in the amino acid sequence as set forth SEQ ID NO: 48. In some cases, the antibody provided herein comprises a heavy chain variable region comprising an amino acid sequence having less than or equal to 1 total substitutions, deletions, and/or insertions in the amino acid sequence as set forth SEQ ID NO: 47, and a light chain variable region comprising an amino acid sequence having less than or equal to 1 total substitutions, deletions, and/or insertions in the amino acid sequence as set forth SEQ ID NO: 48

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 47, and a light chain variable region comprising an amino acid sequence having at least 90%, 95%, 96%, 97%, 98%, 99%, or 100% sequence identity to the amino acid sequence as set forth SEQ ID NO: 49. In some cases, the antibody provided herein comprises a heavy chain variable region comprising an amino acid sequence having at least 95% sequence identity to the amino acid sequence as set forth SEQ ID NO: 47, and a light chain variable region comprising an amino acid sequence having at least 95% sequence identity to the amino acid sequence as set forth SEQ ID NO: 49. In some cases, the antibody provided herein comprises a heavy chain variable region comprising an amino acid sequence having at least 97% sequence identity to the amino acid sequence as set forth SEQ ID NO: 47, and a light chain variable region comprising an amino acid sequence having at least 97% sequence identity to the amino acid sequence as set forth SEQ ID NO: 49. In some cases, the antibody provided herein comprises a heavy chain variable region comprising an amino acid sequence having at least 99% sequence identity to the amino acid sequence as set forth SEQ ID NO: 47, and a light chain variable region comprising an amino acid sequence having at least 99% sequence identity to the amino acid sequence as set forth SEQ ID NO: 49. In some cases, the antibody provided herein comprises a heavy chain variable region comprising an amino acid sequence having 100% sequence identity to the amino acid sequence as set forth SEQ ID NO: 47, and a light chain variable region comprising an amino acid sequence having 100% sequence identity to the amino acid sequence as set forth SEQ ID NO: 49. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 47, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 49. In some cases, the antibody provided herein comprises a heavy chain variable region comprising an amino acid sequence having less than or equal to 10 total substitutions, deletions, and/or insertions in the amino acid sequence as set forth SEQ ID NO: 47, and a light chain variable region comprising an amino acid sequence having less than or equal to 10 total substitutions, deletions, and/or insertions in the amino acid sequence as set forth SEQ ID NO: 49. In some cases, the antibody provided herein comprises a heavy chain variable region comprising an amino acid sequence having less than or equal to 7 total substitutions, deletions, and/or insertions in the amino acid sequence as set forth SEQ ID NO: 47, and a light chain variable region comprising an amino acid sequence having less than or equal to 7 total substitutions, deletions, and/or insertions in the amino acid sequence as set forth SEQ ID NO: 49. In some cases, the antibody provided herein comprises a heavy chain variable region comprising an amino acid sequence having less than or equal to 5 total substitutions, deletions, and/or insertions in the amino acid sequence as set forth SEQ ID NO: 47, and a light chain variable region comprising an amino acid sequence having less than or equal to 5 total substitutions, deletions, and/or insertions in the amino acid sequence as set forth SEQ ID NO: 49. In some cases, the antibody provided herein comprises a heavy chain variable region comprising an amino acid sequence having less than or equal to 3 total substitutions, deletions, and/or insertions in the amino acid sequence as set forth SEQ ID NO: 47, and a light chain variable region comprising an amino acid sequence having less than or equal to 3 total substitutions, deletions, and/or insertions in the amino acid sequence as set forth SEQ ID NO: 49. In some cases, the antibody provided herein comprises a heavy chain variable region comprising an amino acid sequence having less than or equal to 2 total substitutions, deletions, and/or insertions in the amino acid sequence as set forth SEQ ID NO: 47, and a light chain variable region comprising an amino acid sequence having less than or equal to 2 total substitutions, deletions, and/or insertions in the amino acid sequence as set forth SEQ ID NO: 49. In some cases, the antibody provided herein comprises a heavy chain variable region comprising an amino acid sequence having less than or equal to 1 total substitutions, deletions, and/or insertions in the amino acid sequence as set forth SEQ ID NO: 47, and a light chain variable region comprising an amino acid sequence having less than or equal to 1 total substitutions, deletions, and/or insertions in the amino acid sequence as set forth SEQ ID NO: 49.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 47, and a light chain variable region comprising an amino acid sequence having at least 90%, 95%, 96%, 97%, 98%, 99%, or 100% sequence identity to the amino acid sequence as set forth SEQ ID NO: 50. In some cases, the antibody provided herein comprises a heavy chain variable region comprising an amino acid sequence having at least 95% sequence identity to the amino acid sequence as set forth SEQ ID NO: 47, and a light chain variable region comprising an amino acid sequence having at least 95% sequence identity to the amino acid sequence as set forth SEQ ID NO: 50. In some cases, the antibody provided herein comprises a heavy chain variable region comprising an amino acid sequence having at least 97% sequence identity to the amino acid sequence as set forth SEQ ID NO: 47, and a light chain variable region comprising an amino acid sequence having at least 97% sequence identity to the amino acid sequence as set forth SEQ ID NO: 50. In some cases, the antibody provided herein comprises a heavy chain variable region comprising an amino acid sequence having at least 99% sequence identity to the amino acid sequence as set forth SEQ ID NO: 47, and a light chain variable region comprising an amino acid sequence having at least 99% sequence identity to the amino acid sequence as set forth SEQ ID NO: 50. In some cases, the antibody provided herein comprises a heavy chain variable region comprising an amino acid sequence having 100% sequence identity to the amino acid sequence as set forth SEQ ID NO: 47, and a light chain variable region comprising an amino acid sequence having 100% sequence identity to the amino acid sequence as set forth SEQ ID NO: 50. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 47, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 50. In some cases, the antibody provided herein comprises a heavy chain variable region comprising an amino acid sequence having less than or equal to 10 total substitutions, deletions, and/or insertions in the amino acid sequence as set forth SEQ ID NO: 47, and a light chain variable region comprising an amino acid sequence having less than or equal to 10 total substitutions, deletions, and/or insertions in the amino acid sequence as set forth SEQ ID NO: 50. In some cases, the antibody provided herein comprises a heavy chain variable region comprising an amino acid sequence having less than or equal to 7 total substitutions, deletions, and/or insertions in the amino acid sequence as set forth SEQ ID NO: 47, and a light chain variable region comprising an amino acid sequence having less than or equal to 7 total substitutions, deletions, and/or insertions in the amino acid sequence as set forth SEQ ID NO: 50. In some cases, the antibody provided herein comprises a heavy chain variable region comprising an amino acid sequence having less than or equal to 5 total substitutions, deletions, and/or insertions in the amino acid sequence as set forth SEQ ID NO: 47, and a light chain variable region comprising an amino acid sequence having less than or equal to 5 total substitutions, deletions, and/or insertions in the amino acid sequence as set forth SEQ ID NO: 50. In some cases, the antibody provided herein comprises a heavy chain variable region comprising an amino acid sequence having less than or equal to 3 total substitutions, deletions, and/or insertions in the amino acid sequence as set forth SEQ ID NO: 47, and a light chain variable region comprising an amino acid sequence having less than or equal to 3 total substitutions, deletions, and/or insertions in the amino acid sequence as set forth SEQ ID NO: 50. In some cases, the antibody provided herein comprises a heavy chain variable region comprising an amino acid sequence having less than or equal to 2 total substitutions, deletions, and/or insertions in the amino acid sequence as set forth SEQ ID NO: 47, and a light chain variable region comprising an amino acid sequence having less than or equal to 2 total substitutions, deletions, and/or insertions in the amino acid sequence as set forth SEQ ID NO: 50. In some cases, the antibody provided herein comprises a heavy chain variable region comprising an amino acid sequence having less than or equal to 1 total substitutions, deletions, and/or insertions in the amino acid sequence as set forth SEQ ID NO: 47, and a light chain variable region comprising an amino acid sequence having less than or equal to 1 total substitutions, deletions, and/or insertions in the amino acid sequence as set forth SEQ ID NO: 50.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 47, and a light chain variable region comprising an amino acid sequence having at least 90%, 95%, 96%, 97%, 98%, 99%, or 100% sequence identity to the amino acid sequence as set forth SEQ ID NO: 51. In some cases, the antibody provided herein comprises a heavy chain variable region comprising an amino acid sequence having at least 95% sequence identity to the amino acid sequence as set forth SEQ ID NO: 47, and a light chain variable region comprising an amino acid sequence having at least 95% sequence identity to the amino acid sequence as set forth SEQ ID NO: 51. In some cases, the antibody provided herein comprises a heavy chain variable region comprising an amino acid sequence having at least 97% sequence identity to the amino acid sequence as set forth SEQ ID NO: 47, and a light chain variable region comprising an amino acid sequence having at least 97% sequence identity to the amino acid sequence as set forth SEQ ID NO: 51. In some cases, the antibody provided herein comprises a heavy chain variable region comprising an amino acid sequence having at least 99% sequence identity to the amino acid sequence as set forth SEQ ID NO: 47, and a light chain variable region comprising an amino acid sequence having at least 99% sequence identity to the amino acid sequence as set forth SEQ ID NO: 51. In some cases, the antibody provided herein comprises a heavy chain variable region comprising an amino acid sequence having 100% sequence identity to the amino acid sequence as set forth SEQ ID NO: 47, and a light chain variable region comprising an amino acid sequence having 100% sequence identity to the amino acid sequence as set forth SEQ ID NO: 51. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 47, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 51. In some cases, the antibody provided herein comprises a heavy chain variable region comprising an amino acid sequence having less than or equal to 10 total substitutions, deletions, and/or insertions in the amino acid sequence as set forth SEQ ID NO: 47, and a light chain variable region comprising an amino acid sequence having less than or equal to 10 total substitutions, deletions, and/or insertions in the amino acid sequence as set forth SEQ ID NO: 51. In some cases, the antibody provided herein comprises a heavy chain variable region comprising an amino acid sequence having less than or equal to 7 total substitutions, deletions, and/or insertions in the amino acid sequence as set forth SEQ ID NO: 47, and a light chain variable region comprising an amino acid sequence having less than or equal to 7 total substitutions, deletions, and/or insertions in the amino acid sequence as set forth SEQ ID NO: 51. In some cases, the antibody provided herein comprises a heavy chain variable region comprising an amino acid sequence having less than or equal to 5 total substitutions, deletions, and/or insertions in the amino acid sequence as set forth SEQ ID NO: 47, and a light chain variable region comprising an amino acid sequence having less than or equal to 5 total substitutions, deletions, and/or insertions in the amino acid sequence as set forth SEQ ID NO: 51. In some cases, the antibody provided herein comprises a heavy chain variable region comprising an amino acid sequence having less than or equal to 3 total substitutions, deletions, and/or insertions in the amino acid sequence as set forth SEQ ID NO: 47, and a light chain variable region comprising an amino acid sequence having less than or equal to 3 total substitutions, deletions, and/or insertions in the amino acid sequence as set forth SEQ ID NO: 51. In some cases, the antibody provided herein comprises a heavy chain variable region comprising an amino acid sequence having less than or equal to 2 total substitutions, deletions, and/or insertions in the amino acid sequence as set forth SEQ ID NO: 47, and a light chain variable region comprising an amino acid sequence having less than or equal to 2 total substitutions, deletions, and/or insertions in the amino acid sequence as set forth SEQ ID NO: 51. In some cases, the antibody provided herein comprises a heavy chain variable region comprising an amino acid sequence having less than or equal to 1 total substitutions, deletions, and/or insertions in the amino acid sequence as set forth SEQ ID NO: 47, and a light chain variable region comprising an amino acid sequence having less than or equal to 1 total substitutions, deletions, and/or insertions in the amino acid sequence as set forth SEQ ID NO: 51

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 47, and a light chain variable region comprising an amino acid sequence having at least 90%, 95%, 96%, 97%, 98%, 99%, or 100% sequence identity to the amino acid sequence as set forth SEQ ID NO: 52. In some cases, the antibody provided herein comprises a heavy chain variable region comprising an amino acid sequence having at least 95% sequence identity to the amino acid sequence as set forth SEQ ID NO: 47, and a light chain variable region comprising an amino acid sequence having at least 95% sequence identity to the amino acid sequence as set forth SEQ ID NO: 52. In some cases, the antibody provided herein comprises a heavy chain variable region comprising an amino acid sequence having at least 97% sequence identity to the amino acid sequence as set forth SEQ ID NO: 47, and a light chain variable region comprising an amino acid sequence having at least 97% sequence identity to the amino acid sequence as set forth SEQ ID NO: 52. In some cases, the antibody provided herein comprises a heavy chain variable region comprising an amino acid sequence having at least 99% sequence identity to the amino acid sequence as set forth SEQ ID NO: 47, and a light chain variable region comprising an amino acid sequence having at least 99% sequence identity to the amino acid sequence as set forth SEQ ID NO: 52. In some cases, the antibody provided herein comprises a heavy chain variable region comprising an amino acid sequence having 100% sequence identity to the amino acid sequence as set forth SEQ ID NO: 47, and a light chain variable region comprising an amino acid sequence having 100% sequence identity to the amino acid sequence as set forth SEQ ID NO: 52. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 47, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 52. In some cases, the antibody provided herein comprises a heavy chain variable region comprising an amino acid sequence having less than or equal to 10 total substitutions, deletions, and/or insertions in the amino acid sequence as set forth SEQ ID NO: 47, and a light chain variable region comprising an amino acid sequence having less than or equal to 10 total substitutions, deletions, and/or insertions in the amino acid sequence as set forth SEQ ID NO: 52. In some cases, the antibody provided herein comprises a heavy chain variable region comprising an amino acid sequence having less than or equal to 7 total substitutions, deletions, and/or insertions in the amino acid sequence as set forth SEQ ID NO: 47, and a light chain variable region comprising an amino acid sequence having less than or equal to 7 total substitutions, deletions, and/or insertions in the amino acid sequence as set forth SEQ ID NO: 52. In some cases, the antibody provided herein comprises a heavy chain variable region comprising an amino acid sequence having less than or equal to 5 total substitutions, deletions, and/or insertions in the amino acid sequence as set forth SEQ ID NO: 47, and a light chain variable region comprising an amino acid sequence having less than or equal to 5 total substitutions, deletions, and/or insertions in the amino acid sequence as set forth SEQ ID NO: 52. In some cases, the antibody provided herein comprises a heavy chain variable region comprising an amino acid sequence having less than or equal to 3 total substitutions, deletions, and/or insertions in the amino acid sequence as set forth SEQ ID NO: 47, and a light chain variable region comprising an amino acid sequence having less than or equal to 3 total substitutions, deletions, and/or insertions in the amino acid sequence as set forth SEQ ID NO: 52. In some cases, the antibody provided herein comprises a heavy chain variable region comprising an amino acid sequence having less than or equal to 2 total substitutions, deletions, and/or insertions in the amino acid sequence as set forth SEQ ID NO: 47, and a light chain variable region comprising an amino acid sequence having less than or equal to 2 total substitutions, deletions, and/or insertions in the amino acid sequence as set forth SEQ ID NO: 52. In some cases, the antibody provided herein comprises a heavy chain variable region comprising an amino acid sequence having less than or equal to 1 total substitutions, deletions, and/or insertions in the amino acid sequence as set forth SEQ ID NO: 47, and a light chain variable region comprising an amino acid sequence having less than or equal to 1 total substitutions, deletions, and/or insertions in the amino acid sequence as set forth SEQ ID NO: 52.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 47, and a light chain variable region comprising an amino acid sequence having at least 90%, 95%, 96%, 97%, 98%, 99%, or 100% sequence identity to the amino acid sequence as set forth SEQ ID NO: 65. In some cases, the antibody provided herein comprises a heavy chain variable region comprising an amino acid sequence having at least 95% sequence identity to the amino acid sequence as set forth SEQ ID NO: 47, and a light chain variable region comprising an amino acid sequence having at least 95% sequence identity to the amino acid sequence as set forth SEQ ID NO: 65. In some cases, the antibody provided herein comprises a heavy chain variable region comprising an amino acid sequence having at least 97% sequence identity to the amino acid sequence as set forth SEQ ID NO: 47, and a light chain variable region comprising an amino acid sequence having at least 97% sequence identity to the amino acid sequence as set forth SEQ ID NO: 65. In some cases, the antibody provided herein comprises a heavy chain variable region comprising an amino acid sequence having at least 99% sequence identity to the amino acid sequence as set forth SEQ ID NO: 47, and a light chain variable region comprising an amino acid sequence having at least 99% sequence identity to the amino acid sequence as set forth SEQ ID NO: 65. In some cases, the antibody provided herein comprises a heavy chain variable region comprising an amino acid sequence having 100% sequence identity to the amino acid sequence as set forth SEQ ID NO: 47, and a light chain variable region comprising an amino acid sequence having 100% sequence identity to the amino acid sequence as set forth SEQ ID NO: 65. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 47, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 65. In some cases, the antibody provided herein comprises a heavy chain variable region comprising an amino acid sequence having less than or equal to 10 total substitutions, deletions, and/or insertions in the amino acid sequence as set forth SEQ ID NO: 47, and a light chain variable region comprising an amino acid sequence having less than or equal to 10 total substitutions, deletions, and/or insertions in the amino acid sequence as set forth SEQ ID NO: 65. In some cases, the antibody provided herein comprises a heavy chain variable region comprising an amino acid sequence having less than or equal to 7 total substitutions, deletions, and/or insertions in the amino acid sequence as set forth SEQ ID NO: 47, and a light chain variable region comprising an amino acid sequence having less than or equal to 7 total substitutions, deletions, and/or insertions in the amino acid sequence as set forth SEQ ID NO: 65. In some cases, the antibody provided herein comprises a heavy chain variable region comprising an amino acid sequence having less than or equal to 5 total substitutions, deletions, and/or insertions in the amino acid sequence as set forth SEQ ID NO: 47, and a light chain variable region comprising an amino acid sequence having less than or equal to 5 total substitutions, deletions, and/or insertions in the amino acid sequence as set forth SEQ ID NO: 65. In some cases, the antibody provided herein comprises a heavy chain variable region comprising an amino acid sequence having less than or equal to 3 total substitutions, deletions, and/or insertions in the amino acid sequence as set forth SEQ ID NO: 47, and a light chain variable region comprising an amino acid sequence having less than or equal to 3 total substitutions, deletions, and/or insertions in the amino acid sequence as set forth SEQ ID NO: 65. In some cases, the antibody provided herein comprises a heavy chain variable region comprising an amino acid sequence having less than or equal to 2 total substitutions, deletions, and/or insertions in the amino acid sequence as set forth SEQ ID NO: 47, and a light chain variable region comprising an amino acid sequence having less than or equal to 2 total substitutions, deletions, and/or insertions in the amino acid sequence as set forth SEQ ID NO: 65. In some cases, the antibody provided herein comprises a heavy chain variable region comprising an amino acid sequence having less than or equal to 1 total substitutions, deletions, and/or insertions in the amino acid sequence as set forth SEQ ID NO: 47, and a light chain variable region comprising an amino acid sequence having less than or equal to 1 total substitutions, deletions, and/or insertions in the amino acid sequence as set forth SEQ ID NO: 65.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 47, and a light chain variable region comprising an amino acid sequence having at least 90%, 95%, 96%, 97%, 98%, 99%, or 100% sequence identity to the amino acid sequence as set forth SEQ ID NO: 66. In some cases, the antibody provided herein comprises a heavy chain variable region comprising an amino acid sequence having at least 95% sequence identity to the amino acid sequence as set forth SEQ ID NO: 47, and a light chain variable region comprising an amino acid sequence having at least 95% sequence identity to the amino acid sequence as set forth SEQ ID NO: 66. In some cases, the antibody provided herein comprises a heavy chain variable region comprising an amino acid sequence having at least 97% sequence identity to the amino acid sequence as set forth SEQ ID NO: 47, and a light chain variable region comprising an amino acid sequence having at least 97% sequence identity to the amino acid sequence as set forth SEQ ID NO: 66. In some cases, the antibody provided herein comprises a heavy chain variable region comprising an amino acid sequence having at least 99% sequence identity to the amino acid sequence as set forth SEQ ID NO: 47, and a light chain variable region comprising an amino acid sequence having at least 99% sequence identity to the amino acid sequence as set forth SEQ ID NO: 66. In some cases, the antibody provided herein comprises a heavy chain variable region comprising an amino acid sequence having 100% sequence identity to the amino acid sequence as set forth SEQ ID NO: 47, and a light chain variable region comprising an amino acid sequence having 100% sequence identity to the amino acid sequence as set forth SEQ ID NO: 66. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 47, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 66. In some cases, the antibody provided herein comprises a heavy chain variable region comprising an amino acid sequence having less than or equal to 10 total substitutions, deletions, and/or insertions in the amino acid sequence as set forth SEQ ID NO: 47, and a light chain variable region comprising an amino acid sequence having less than or equal to 10 total substitutions, deletions, and/or insertions in the amino acid sequence as set forth SEQ ID NO: 66. In some cases, the antibody provided herein comprises a heavy chain variable region comprising an amino acid sequence having less than or equal to 7 total substitutions, deletions, and/or insertions in the amino acid sequence as set forth SEQ ID NO: 47, and a light chain variable region comprising an amino acid sequence having less than or equal to 7 total substitutions, deletions, and/or insertions in the amino acid sequence as set forth SEQ ID NO: 66. In some cases, the antibody provided herein comprises a heavy chain variable region comprising an amino acid sequence having less than or equal to 5 total substitutions, deletions, and/or insertions in the amino acid sequence as set forth SEQ ID NO: 47, and a light chain variable region comprising an amino acid sequence having less than or equal to 5 total substitutions, deletions, and/or insertions in the amino acid sequence as set forth SEQ ID NO: 66. In some cases, the antibody provided herein comprises a heavy chain variable region comprising an amino acid sequence having less than or equal to 3 total substitutions, deletions, and/or insertions in the amino acid sequence as set forth SEQ ID NO: 47, and a light chain variable region comprising an amino acid sequence having less than or equal to 3 total substitutions, deletions, and/or insertions in the amino acid sequence as set forth SEQ ID NO: 66. In some cases, the antibody provided herein comprises a heavy chain variable region comprising an amino acid sequence having less than or equal to 2 total substitutions, deletions, and/or insertions in the amino acid sequence as set forth SEQ ID NO: 47, and a light chain variable region comprising an amino acid sequence having less than or equal to 2 total substitutions, deletions, and/or insertions in the amino acid sequence as set forth SEQ ID NO: 66. In some cases, the antibody provided herein comprises a heavy chain variable region comprising an amino acid sequence having less than or equal to 1 total substitutions, deletions, and/or insertions in the amino acid sequence as set forth SEQ ID NO: 47, and a light chain variable region comprising an amino acid sequence having less than or equal to 1 total substitutions, deletions, and/or insertions in the amino acid sequence as set forth SEQ ID NO: 66.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 71, and a light chain variable region comprising an amino acid sequence having at least 90%, 95%, 96%, 97%, 98%, 99%, or 100% sequence identity to the amino acid sequence as set forth SEQ ID NO: 2. In some cases, the antibody provided herein comprises a heavy chain variable region comprising an amino acid sequence having at least 95% sequence identity to the amino acid sequence as set forth SEQ ID NO: 71, and a light chain variable region comprising an amino acid sequence having at least 95% sequence identity to the amino acid sequence as set forth SEQ ID NO: 2. In some cases, the antibody provided herein comprises a heavy chain variable region comprising an amino acid sequence having at least 97% sequence identity to the amino acid sequence as set forth SEQ ID NO: 71, and a light chain variable region comprising an amino acid sequence having at least 97% sequence identity to the amino acid sequence as set forth SEQ ID NO: 2. In some cases, the antibody provided herein comprises a heavy chain variable region comprising an amino acid sequence having at least 99% sequence identity to the amino acid sequence as set forth SEQ ID NO: 71, and a light chain variable region comprising an amino acid sequence having at least 99% sequence identity to the amino acid sequence as set forth SEQ ID NO: 2. In some cases, the antibody provided herein comprises a heavy chain variable region comprising an amino acid sequence having 100% sequence identity to the amino acid sequence as set forth SEQ ID NO: 71, and a light chain variable region comprising an amino acid sequence having 100% sequence identity to the amino acid sequence as set forth SEQ ID NO: 2. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 71, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 2. In some cases, the antibody provided herein comprises a heavy chain variable region comprising an amino acid sequence having less than or equal to 10 total substitutions, deletions, and/or insertions in the amino acid sequence as set forth SEQ ID NO: 71, and a light chain variable region comprising an amino acid sequence having less than or equal to 10 total substitutions, deletions, and/or insertions in the amino acid sequence as set forth SEQ ID NO: 2. In some cases, the antibody provided herein comprises a heavy chain variable region comprising an amino acid sequence having less than or equal to 7 total substitutions, deletions, and/or insertions in the amino acid sequence as set forth SEQ ID NO: 71, and a light chain variable region comprising an amino acid sequence having less than or equal to 7 total substitutions, deletions, and/or insertions in the amino acid sequence as set forth SEQ ID NO: 2. In some cases, the antibody provided herein comprises a heavy chain variable region comprising an amino acid sequence having less than or equal to 5 total substitutions, deletions, and/or insertions in the amino acid sequence as set forth SEQ ID NO: 71, and a light chain variable region comprising an amino acid sequence having less than or equal to 5 total substitutions, deletions, and/or insertions in the amino acid sequence as set forth SEQ ID NO: 2. In some cases, the antibody provided herein comprises a heavy chain variable region comprising an amino acid sequence having less than or equal to 3 total substitutions, deletions, and/or insertions in the amino acid sequence as set forth SEQ ID NO: 71, and a light chain variable region comprising an amino acid sequence having less than or equal to 3 total substitutions, deletions, and/or insertions in the amino acid sequence as set forth SEQ ID NO: 2. In some cases, the antibody provided herein comprises a heavy chain variable region comprising an amino acid sequence having less than or equal to 2 total substitutions, deletions, and/or insertions in the amino acid sequence as set forth SEQ ID NO: 71, and a light chain variable region comprising an amino acid sequence having less than or equal to 2 total substitutions, deletions, and/or insertions in the amino acid sequence as set forth SEQ ID NO: 2. In some cases, the antibody provided herein comprises a heavy chain variable region comprising an amino acid sequence having less than or equal to 1 total substitutions, deletions, and/or insertions in the amino acid sequence as set forth SEQ ID NO: 71, and a light chain variable region comprising an amino acid sequence having less than or equal to 1 total substitutions, deletions, and/or insertions in the amino acid sequence as set forth SEQ ID NO: 2.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 71, and a light chain variable region comprising an amino acid sequence having at least 90%, 95%, 96%, 97%, 98%, 99%, or 100% sequence identity to the amino acid sequence as set forth SEQ ID NO: 48. In some cases, the antibody provided herein comprises a heavy chain variable region comprising an amino acid sequence having at least 95% sequence identity to the amino acid sequence as set forth SEQ ID NO: 71, and a light chain variable region comprising an amino acid sequence having at least 95% sequence identity to the amino acid sequence as set forth SEQ ID NO: 48. In some cases, the antibody provided herein comprises a heavy chain variable region comprising an amino acid sequence having at least 97% sequence identity to the amino acid sequence as set forth SEQ ID NO: 71, and a light chain variable region comprising an amino acid sequence having at least 97% sequence identity to the amino acid sequence as set forth SEQ ID NO: 48. In some cases, the antibody provided herein comprises a heavy chain variable region comprising an amino acid sequence having at least 99% sequence identity to the amino acid sequence as set forth SEQ ID NO: 71, and a light chain variable region comprising an amino acid sequence having at least 99% sequence identity to the amino acid sequence as set forth SEQ ID NO: 48. In some cases, the antibody provided herein comprises a heavy chain variable region comprising an amino acid sequence having 100% sequence identity to the amino acid sequence as set forth SEQ ID NO: 71, and a light chain variable region comprising an amino acid sequence having 100% sequence identity to the amino acid sequence as set forth SEQ ID NO: 48. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 71, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 48. In some cases, the antibody provided herein comprises a heavy chain variable region comprising an amino acid sequence having less than or equal to 10 total substitutions, deletions, and/or insertions in the amino acid sequence as set forth SEQ ID NO: 71, and a light chain variable region comprising an amino acid sequence having less than or equal to 10 total substitutions, deletions, and/or insertions in the amino acid sequence as set forth SEQ ID NO: 48. In some cases, the antibody provided herein comprises a heavy chain variable region comprising an amino acid sequence having less than or equal to 7 total substitutions, deletions, and/or insertions in the amino acid sequence as set forth SEQ ID NO: 71, and a light chain variable region comprising an amino acid sequence having less than or equal to 7 total substitutions, deletions, and/or insertions in the amino acid sequence as set forth SEQ ID NO: 48. In some cases, the antibody provided herein comprises a heavy chain variable region comprising an amino acid sequence having less than or equal to 5 total substitutions, deletions, and/or insertions in the amino acid sequence as set forth SEQ ID NO: 71, and a light chain variable region comprising an amino acid sequence having less than or equal to 5 total substitutions, deletions, and/or insertions in the amino acid sequence as set forth SEQ ID NO: 48. In some cases, the antibody provided herein comprises a heavy chain variable region comprising an amino acid sequence having less than or equal to 3 total substitutions, deletions, and/or insertions in the amino acid sequence as set forth SEQ ID NO: 71, and a light chain variable region comprising an amino acid sequence having less than or equal to 3 total substitutions, deletions, and/or insertions in the amino acid sequence as set forth SEQ ID NO: 48. In some cases, the antibody provided herein comprises a heavy chain variable region comprising an amino acid sequence having less than or equal to 2 total substitutions, deletions, and/or insertions in the amino acid sequence as set forth SEQ ID NO: 71, and a light chain variable region comprising an amino acid sequence having less than or equal to 2 total substitutions, deletions, and/or insertions in the amino acid sequence as set forth SEQ ID NO: 48. In some cases, the antibody provided herein comprises a heavy chain variable region comprising an amino acid sequence having less than or equal to 1 total substitutions, deletions, and/or insertions in the amino acid sequence as set forth SEQ ID NO: 71, and a light chain variable region comprising an amino acid sequence having less than or equal to 1 total substitutions, deletions, and/or insertions in the amino acid sequence as set forth SEQ ID NO: 48

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 71, and a light chain variable region comprising an amino acid sequence having at least 90%, 95%, 96%, 97%, 98%, 99%, or 100% sequence identity to the amino acid sequence as set forth SEQ ID NO: 49. In some cases, the antibody provided herein comprises a heavy chain variable region comprising an amino acid sequence having at least 95% sequence identity to the amino acid sequence as set forth SEQ ID NO: 71, and a light chain variable region comprising an amino acid sequence having at least 95% sequence identity to the amino acid sequence as set forth SEQ ID NO: 49. In some cases, the antibody provided herein comprises a heavy chain variable region comprising an amino acid sequence having at least 97% sequence identity to the amino acid sequence as set forth SEQ ID NO: 71, and a light chain variable region comprising an amino acid sequence having at least 97% sequence identity to the amino acid sequence as set forth SEQ ID NO: 49. In some cases, the antibody provided herein comprises a heavy chain variable region comprising an amino acid sequence having at least 99% sequence identity to the amino acid sequence as set forth SEQ ID NO: 71, and a light chain variable region comprising an amino acid sequence having at least 99% sequence identity to the amino acid sequence as set forth SEQ ID NO: 49. In some cases, the antibody provided herein comprises a heavy chain variable region comprising an amino acid sequence having 100% sequence identity to the amino acid sequence as set forth SEQ ID NO: 71, and a light chain variable region comprising an amino acid sequence having 100% sequence identity to the amino acid sequence as set forth SEQ ID NO: 49. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 71, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 49. In some cases, the antibody provided herein comprises a heavy chain variable region comprising an amino acid sequence having less than or equal to 10 total substitutions, deletions, and/or insertions in the amino acid sequence as set forth SEQ ID NO: 71, and a light chain variable region comprising an amino acid sequence having less than or equal to 10 total substitutions, deletions, and/or insertions in the amino acid sequence as set forth SEQ ID NO: 49. In some cases, the antibody provided herein comprises a heavy chain variable region comprising an amino acid sequence having less than or equal to 7 total substitutions, deletions, and/or insertions in the amino acid sequence as set forth SEQ ID NO: 71, and a light chain variable region comprising an amino acid sequence having less than or equal to 7 total substitutions, deletions, and/or insertions in the amino acid sequence as set forth SEQ ID NO: 49. In some cases, the antibody provided herein comprises a heavy chain variable region comprising an amino acid sequence having less than or equal to 5 total substitutions, deletions, and/or insertions in the amino acid sequence as set forth SEQ ID NO: 71, and a light chain variable region comprising an amino acid sequence having less than or equal to 5 total substitutions, deletions, and/or insertions in the amino acid sequence as set forth SEQ ID NO: 49. In some cases, the antibody provided herein comprises a heavy chain variable region comprising an amino acid sequence having less than or equal to 3 total substitutions, deletions, and/or insertions in the amino acid sequence as set forth SEQ ID NO: 71, and a light chain variable region comprising an amino acid sequence having less than or equal to 3 total substitutions, deletions, and/or insertions in the amino acid sequence as set forth SEQ ID NO: 49. In some cases, the antibody provided herein comprises a heavy chain variable region comprising an amino acid sequence having less than or equal to 2 total substitutions, deletions, and/or insertions in the amino acid sequence as set forth SEQ ID NO: 71, and a light chain variable region comprising an amino acid sequence having less than or equal to 2 total substitutions, deletions, and/or insertions in the amino acid sequence as set forth SEQ ID NO: 49. In some cases, the antibody provided herein comprises a heavy chain variable region comprising an amino acid sequence having less than or equal to 1 total substitutions, deletions, and/or insertions in the amino acid sequence as set forth SEQ ID NO: 71, and a light chain variable region comprising an amino acid sequence having less than or equal to 1 total substitutions, deletions, and/or insertions in the amino acid sequence as set forth SEQ ID NO: 49.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 71, and a light chain variable region comprising an amino acid sequence having at least 90%, 95%, 96%, 97%, 98%, 99%, or 100% sequence identity to the amino acid sequence as set forth SEQ ID NO: 50. In some cases, the antibody provided herein comprises a heavy chain variable region comprising an amino acid sequence having at least 95% sequence identity to the amino acid sequence as set forth SEQ ID NO: 71, and a light chain variable region comprising an amino acid sequence having at least 95% sequence identity to the amino acid sequence as set forth SEQ ID NO: 50. In some cases, the antibody provided herein comprises a heavy chain variable region comprising an amino acid sequence having at least 97% sequence identity to the amino acid sequence as set forth SEQ ID NO: 71, and a light chain variable region comprising an amino acid sequence having at least 97% sequence identity to the amino acid sequence as set forth SEQ ID NO: 50. In some cases, the antibody provided herein comprises a heavy chain variable region comprising an amino acid sequence having at least 99% sequence identity to the amino acid sequence as set forth SEQ ID NO: 71, and a light chain variable region comprising an amino acid sequence having at least 99% sequence identity to the amino acid sequence as set forth SEQ ID NO: 50. In some cases, the antibody provided herein comprises a heavy chain variable region comprising an amino acid sequence having 100% sequence identity to the amino acid sequence as set forth SEQ ID NO: 71, and a light chain variable region comprising an amino acid sequence having 100% sequence identity to the amino acid sequence as set forth SEQ ID NO: 50. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 71, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 50. In some cases, the antibody provided herein comprises a heavy chain variable region comprising an amino acid sequence having less than or equal to 10 total substitutions, deletions, and/or insertions in the amino acid sequence as set forth SEQ ID NO: 71, and a light chain variable region comprising an amino acid sequence having less than or equal to 10 total substitutions, deletions, and/or insertions in the amino acid sequence as set forth SEQ ID NO: 50. In some cases, the antibody provided herein comprises a heavy chain variable region comprising an amino acid sequence having less than or equal to 7 total substitutions, deletions, and/or insertions in the amino acid sequence as set forth SEQ ID NO: 71, and a light chain variable region comprising an amino acid sequence having less than or equal to 7 total substitutions, deletions, and/or insertions in the amino acid sequence as set forth SEQ ID NO: 50. In some cases, the antibody provided herein comprises a heavy chain variable region comprising an amino acid sequence having less than or equal to 5 total substitutions, deletions, and/or insertions in the amino acid sequence as set forth SEQ ID NO: 71, and a light chain variable region comprising an amino acid sequence having less than or equal to 5 total substitutions, deletions, and/or insertions in the amino acid sequence as set forth SEQ ID NO: 50. In some cases, the antibody provided herein comprises a heavy chain variable region comprising an amino acid sequence having less than or equal to 3 total substitutions, deletions, and/or insertions in the amino acid sequence as set forth SEQ ID NO: 71, and a light chain variable region comprising an amino acid sequence having less than or equal to 3 total substitutions, deletions, and/or insertions in the amino acid sequence as set forth SEQ ID NO: 50. In some cases, the antibody provided herein comprises a heavy chain variable region comprising an amino acid sequence having less than or equal to 2 total substitutions, deletions, and/or insertions in the amino acid sequence as set forth SEQ ID NO: 71, and a light chain variable region comprising an amino acid sequence having less than or equal to 2 total substitutions, deletions, and/or insertions in the amino acid sequence as set forth SEQ ID NO: 50. In some cases, the antibody provided herein comprises a heavy chain variable region comprising an amino acid sequence having less than or equal to 1 total substitutions, deletions, and/or insertions in the amino acid sequence as set forth SEQ ID NO: 71, and a light chain variable region comprising an amino acid sequence having less than or equal to 1 total substitutions, deletions, and/or insertions in the amino acid sequence as set forth SEQ ID NO: 50.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 71, and a light chain variable region comprising an amino acid sequence having at least 90%, 95%, 96%, 97%, 98%, 99%, or 100% sequence identity to the amino acid sequence as set forth SEQ ID NO: 51. In some cases, the antibody provided herein comprises a heavy chain variable region comprising an amino acid sequence having at least 95% sequence identity to the amino acid sequence as set forth SEQ ID NO: 71, and a light chain variable region comprising an amino acid sequence having at least 95% sequence identity to the amino acid sequence as set forth SEQ ID NO: 51. In some cases, the antibody provided herein comprises a heavy chain variable region comprising an amino acid sequence having at least 97% sequence identity to the amino acid sequence as set forth SEQ ID NO: 71, and a light chain variable region comprising an amino acid sequence having at least 97% sequence identity to the amino acid sequence as set forth SEQ ID NO: 51. In some cases, the antibody provided herein comprises a heavy chain variable region comprising an amino acid sequence having at least 99% sequence identity to the amino acid sequence as set forth SEQ ID NO: 71, and a light chain variable region comprising an amino acid sequence having at least 99% sequence identity to the amino acid sequence as set forth SEQ ID NO: 51. In some cases, the antibody provided herein comprises a heavy chain variable region comprising an amino acid sequence having 100% sequence identity to the amino acid sequence as set forth SEQ ID NO: 71, and a light chain variable region comprising an amino acid sequence having 100% sequence identity to the amino acid sequence as set forth SEQ ID NO: 51. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 71, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 51. In some cases, the antibody provided herein comprises a heavy chain variable region comprising an amino acid sequence having less than or equal to 10 total substitutions, deletions, and/or insertions in the amino acid sequence as set forth SEQ ID NO: 71, and a light chain variable region comprising an amino acid sequence having less than or equal to 10 total substitutions, deletions, and/or insertions in the amino acid sequence as set forth SEQ ID NO: 51. In some cases, the antibody provided herein comprises a heavy chain variable region comprising an amino acid sequence having less than or equal to 7 total substitutions, deletions, and/or insertions in the amino acid sequence as set forth SEQ ID NO: 71, and a light chain variable region comprising an amino acid sequence having less than or equal to 7 total substitutions, deletions, and/or insertions in the amino acid sequence as set forth SEQ ID NO: 51. In some cases, the antibody provided herein comprises a heavy chain variable region comprising an amino acid sequence having less than or equal to 5 total substitutions, deletions, and/or insertions in the amino acid sequence as set forth SEQ ID NO: 71, and a light chain variable region comprising an amino acid sequence having less than or equal to 5 total substitutions, deletions, and/or insertions in the amino acid sequence as set forth SEQ ID NO: 51. In some cases, the antibody provided herein comprises a heavy chain variable region comprising an amino acid sequence having less than or equal to 3 total substitutions, deletions, and/or insertions in the amino acid sequence as set forth SEQ ID NO: 71, and a light chain variable region comprising an amino acid sequence having less than or equal to 3 total substitutions, deletions, and/or insertions in the amino acid sequence as set forth SEQ ID NO: 51. In some cases, the antibody provided herein comprises a heavy chain variable region comprising an amino acid sequence having less than or equal to 2 total substitutions, deletions, and/or insertions in the amino acid sequence as set forth SEQ ID NO: 71, and a light chain variable region comprising an amino acid sequence having less than or equal to 2 total substitutions, deletions, and/or insertions in the amino acid sequence as set forth SEQ ID NO: 51. In some cases, the antibody provided herein comprises a heavy chain variable region comprising an amino acid sequence having less than or equal to 1 total substitutions, deletions, and/or insertions in the amino acid sequence as set forth SEQ ID NO: 71, and a light chain variable region comprising an amino acid sequence having less than or equal to 1 total substitutions, deletions, and/or insertions in the amino acid sequence as set forth SEQ ID NO: 51

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 71, and a light chain variable region comprising an amino acid sequence having at least 90%, 95%, 96%, 97%, 98%, 99%, or 100% sequence identity to the amino acid sequence as set forth SEQ ID NO: 52. In some cases, the antibody provided herein comprises a heavy chain variable region comprising an amino acid sequence having at least 95% sequence identity to the amino acid sequence as set forth SEQ ID NO: 71, and a light chain variable region comprising an amino acid sequence having at least 95% sequence identity to the amino acid sequence as set forth SEQ ID NO: 52. In some cases, the antibody provided herein comprises a heavy chain variable region comprising an amino acid sequence having at least 97% sequence identity to the amino acid sequence as set forth SEQ ID NO: 71, and a light chain variable region comprising an amino acid sequence having at least 97% sequence identity to the amino acid sequence as set forth SEQ ID NO: 52. In some cases, the antibody provided herein comprises a heavy chain variable region comprising an amino acid sequence having at least 99% sequence identity to the amino acid sequence as set forth SEQ ID NO: 71, and a light chain variable region comprising an amino acid sequence having at least 99% sequence identity to the amino acid sequence as set forth SEQ ID NO: 52. In some cases, the antibody provided herein comprises a heavy chain variable region comprising an amino acid sequence having 100% sequence identity to the amino acid sequence as set forth SEQ ID NO: 71, and a light chain variable region comprising an amino acid sequence having 100% sequence identity to the amino acid sequence as set forth SEQ ID NO: 52. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 71, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 52. In some cases, the antibody provided herein comprises a heavy chain variable region comprising an amino acid sequence having less than or equal to 10 total substitutions, deletions, and/or insertions in the amino acid sequence as set forth SEQ ID NO: 71, and a light chain variable region comprising an amino acid sequence having less than or equal to 10 total substitutions, deletions, and/or insertions in the amino acid sequence as set forth SEQ ID NO: 52. In some cases, the antibody provided herein comprises a heavy chain variable region comprising an amino acid sequence having less than or equal to 7 total substitutions, deletions, and/or insertions in the amino acid sequence as set forth SEQ ID NO: 71, and a light chain variable region comprising an amino acid sequence having less than or equal to 7 total substitutions, deletions, and/or insertions in the amino acid sequence as set forth SEQ ID NO: 52. In some cases, the antibody provided herein comprises a heavy chain variable region comprising an amino acid sequence having less than or equal to 5 total substitutions, deletions, and/or insertions in the amino acid sequence as set forth SEQ ID NO: 71, and a light chain variable region comprising an amino acid sequence having less than or equal to 5 total substitutions, deletions, and/or insertions in the amino acid sequence as set forth SEQ ID NO: 52. In some cases, the antibody provided herein comprises a heavy chain variable region comprising an amino acid sequence having less than or equal to 3 total substitutions, deletions, and/or insertions in the amino acid sequence as set forth SEQ ID NO: 71, and a light chain variable region comprising an amino acid sequence having less than or equal to 3 total substitutions, deletions, and/or insertions in the amino acid sequence as set forth SEQ ID NO: 52. In some cases, the antibody provided herein comprises a heavy chain variable region comprising an amino acid sequence having less than or equal to 2 total substitutions, deletions, and/or insertions in the amino acid sequence as set forth SEQ ID NO: 71, and a light chain variable region comprising an amino acid sequence having less than or equal to 2 total substitutions, deletions, and/or insertions in the amino acid sequence as set forth SEQ ID NO: 52. In some cases, the antibody provided herein comprises a heavy chain variable region comprising an amino acid sequence having less than or equal to 1 total substitutions, deletions, and/or insertions in the amino acid sequence as set forth SEQ ID NO: 71, and a light chain variable region comprising an amino acid sequence having less than or equal to 1 total substitutions, deletions, and/or insertions in the amino acid sequence as set forth SEQ ID NO: 52.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 71, and a light chain variable region comprising an amino acid sequence having at least 90%, 95%, 96%, 97%, 98%, 99%, or 100% sequence identity to the amino acid sequence as set forth SEQ ID NO: 65. In some cases, the antibody provided herein comprises a heavy chain variable region comprising an amino acid sequence having at least 95% sequence identity to the amino acid sequence as set forth SEQ ID NO: 71, and a light chain variable region comprising an amino acid sequence having at least 95% sequence identity to the amino acid sequence as set forth SEQ ID NO: 65. In some cases, the antibody provided herein comprises a heavy chain variable region comprising an amino acid sequence having at least 97% sequence identity to the amino acid sequence as set forth SEQ ID NO: 71, and a light chain variable region comprising an amino acid sequence having at least 97% sequence identity to the amino acid sequence as set forth SEQ ID NO: 65. In some cases, the antibody provided herein comprises a heavy chain variable region comprising an amino acid sequence having at least 99% sequence identity to the amino acid sequence as set forth SEQ ID NO: 71, and a light chain variable region comprising an amino acid sequence having at least 99% sequence identity to the amino acid sequence as set forth SEQ ID NO: 65. In some cases, the antibody provided herein comprises a heavy chain variable region comprising an amino acid sequence having 100% sequence identity to the amino acid sequence as set forth SEQ ID NO: 71, and a light chain variable region comprising an amino acid sequence having 100% sequence identity to the amino acid sequence as set forth SEQ ID NO: 65. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 71, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 65. In some cases, the antibody provided herein comprises a heavy chain variable region comprising an amino acid sequence having less than or equal to 10 total substitutions, deletions, and/or insertions in the amino acid sequence as set forth SEQ ID NO: 71, and a light chain variable region comprising an amino acid sequence having less than or equal to 10 total substitutions, deletions, and/or insertions in the amino acid sequence as set forth SEQ ID NO: 65. In some cases, the antibody provided herein comprises a heavy chain variable region comprising an amino acid sequence having less than or equal to 7 total substitutions, deletions, and/or insertions in the amino acid sequence as set forth SEQ ID NO: 71, and a light chain variable region comprising an amino acid sequence having less than or equal to 7 total substitutions, deletions, and/or insertions in the amino acid sequence as set forth SEQ ID NO: 65. In some cases, the antibody provided herein comprises a heavy chain variable region comprising an amino acid sequence having less than or equal to 5 total substitutions, deletions, and/or insertions in the amino acid sequence as set forth SEQ ID NO: 71, and a light chain variable region comprising an amino acid sequence having less than or equal to 5 total substitutions, deletions, and/or insertions in the amino acid sequence as set forth SEQ ID NO: 65. In some cases, the antibody provided herein comprises a heavy chain variable region comprising an amino acid sequence having less than or equal to 3 total substitutions, deletions, and/or insertions in the amino acid sequence as set forth SEQ ID NO: 71, and a light chain variable region comprising an amino acid sequence having less than or equal to 3 total substitutions, deletions, and/or insertions in the amino acid sequence as set forth SEQ ID NO: 65. In some cases, the antibody provided herein comprises a heavy chain variable region comprising an amino acid sequence having less than or equal to 2 total substitutions, deletions, and/or insertions in the amino acid sequence as set forth SEQ ID NO: 71, and a light chain variable region comprising an amino acid sequence having less than or equal to 2 total substitutions, deletions, and/or insertions in the amino acid sequence as set forth SEQ ID NO: 65. In some cases, the antibody provided herein comprises a heavy chain variable region comprising an amino acid sequence having less than or equal to 1 total substitutions, deletions, and/or insertions in the amino acid sequence as set forth SEQ ID NO: 71, and a light chain variable region comprising an amino acid sequence having less than or equal to 1 total substitutions, deletions, and/or insertions in the amino acid sequence as set forth SEQ ID NO: 65.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 71, and a light chain variable region comprising an amino acid sequence having at least 90%, 95%, 96%, 97%, 98%, 99%, or 100% sequence identity to the amino acid sequence as set forth SEQ ID NO: 66. In some cases, the antibody provided herein comprises a heavy chain variable region comprising an amino acid sequence having at least 95% sequence identity to the amino acid sequence as set forth SEQ ID NO: 71, and a light chain variable region comprising an amino acid sequence having at least 95% sequence identity to the amino acid sequence as set forth SEQ ID NO: 66. In some cases, the antibody provided herein comprises a heavy chain variable region comprising an amino acid sequence having at least 97% sequence identity to the amino acid sequence as set forth SEQ ID NO: 71, and a light chain variable region comprising an amino acid sequence having at least 97% sequence identity to the amino acid sequence as set forth SEQ ID NO: 66. In some cases, the antibody provided herein comprises a heavy chain variable region comprising an amino acid sequence having at least 99% sequence identity to the amino acid sequence as set forth SEQ ID NO: 71, and a light chain variable region comprising an amino acid sequence having at least 99% sequence identity to the amino acid sequence as set forth SEQ ID NO: 66. In some cases, the antibody provided herein comprises a heavy chain variable region comprising an amino acid sequence having 100% sequence identity to the amino acid sequence as set forth SEQ ID NO: 71, and a light chain variable region comprising an amino acid sequence having 100% sequence identity to the amino acid sequence as set forth SEQ ID NO: 66. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 71, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 66. In some cases, the antibody provided herein comprises a heavy chain variable region comprising an amino acid sequence having less than or equal to 10 total substitutions, deletions, and/or insertions in the amino acid sequence as set forth SEQ ID NO: 71, and a light chain variable region comprising an amino acid sequence having less than or equal to 10 total substitutions, deletions, and/or insertions in the amino acid sequence as set forth SEQ ID NO: 66. In some cases, the antibody provided herein comprises a heavy chain variable region comprising an amino acid sequence having less than or equal to 7 total substitutions, deletions, and/or insertions in the amino acid sequence as set forth SEQ ID NO: 71, and a light chain variable region comprising an amino acid sequence having less than or equal to 7 total substitutions, deletions, and/or insertions in the amino acid sequence as set forth SEQ ID NO: 66. In some cases, the antibody provided herein comprises a heavy chain variable region comprising an amino acid sequence having less than or equal to 5 total substitutions, deletions, and/or insertions in the amino acid sequence as set forth SEQ ID NO: 71, and a light chain variable region comprising an amino acid sequence having less than or equal to 5 total substitutions, deletions, and/or insertions in the amino acid sequence as set forth SEQ ID NO: 66. In some cases, the antibody provided herein comprises a heavy chain variable region comprising an amino acid sequence having less than or equal to 3 total substitutions, deletions, and/or insertions in the amino acid sequence as set forth SEQ ID NO: 71, and a light chain variable region comprising an amino acid sequence having less than or equal to 3 total substitutions, deletions, and/or insertions in the amino acid sequence as set forth SEQ ID NO: 66. In some cases, the antibody provided herein comprises a heavy chain variable region comprising an amino acid sequence having less than or equal to 2 total substitutions, deletions, and/or insertions in the amino acid sequence as set forth SEQ ID NO: 71, and a light chain variable region comprising an amino acid sequence having less than or equal to 2 total substitutions, deletions, and/or insertions in the amino acid sequence as set forth SEQ ID NO: 66. In some cases, the antibody provided herein comprises a heavy chain variable region comprising an amino acid sequence having less than or equal to 1 total substitutions, deletions, and/or insertions in the amino acid sequence as set forth SEQ ID NO: 71, and a light chain variable region comprising an amino acid sequence having less than or equal to 1 total substitutions, deletions, and/or insertions in the amino acid sequence as set forth SEQ ID NO: 66.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 72, and a light chain variable region comprising an amino acid sequence having at least 90%, 95%, 96%, 97%, 98%, 99%, or 100% sequence identity to the amino acid sequence as set forth SEQ ID NO: 2. In some cases, the antibody provided herein comprises a heavy chain variable region comprising an amino acid sequence having at least 95% sequence identity to the amino acid sequence as set forth SEQ ID NO: 72, and a light chain variable region comprising an amino acid sequence having at least 95% sequence identity to the amino acid sequence as set forth SEQ ID NO: 2. In some cases, the antibody provided herein comprises a heavy chain variable region comprising an amino acid sequence having at least 97% sequence identity to the amino acid sequence as set forth SEQ ID NO: 72, and a light chain variable region comprising an amino acid sequence having at least 97% sequence identity to the amino acid sequence as set forth SEQ ID NO: 2. In some cases, the antibody provided herein comprises a heavy chain variable region comprising an amino acid sequence having at least 99% sequence identity to the amino acid sequence as set forth SEQ ID NO: 72, and a light chain variable region comprising an amino acid sequence having at least 99% sequence identity to the amino acid sequence as set forth SEQ ID NO: 2. In some cases, the antibody provided herein comprises a heavy chain variable region comprising an amino acid sequence having 100% sequence identity to the amino acid sequence as set forth SEQ ID NO: 72, and a light chain variable region comprising an amino acid sequence having 100% sequence identity to the amino acid sequence as set forth SEQ ID NO: 2. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 72, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 2. In some cases, the antibody provided herein comprises a heavy chain variable region comprising an amino acid sequence having less than or equal to 10 total substitutions, deletions, and/or insertions in the amino acid sequence as set forth SEQ ID NO: 72, and a light chain variable region comprising an amino acid sequence having less than or equal to 10 total substitutions, deletions, and/or insertions in the amino acid sequence as set forth SEQ ID NO: 2. In some cases, the antibody provided herein comprises a heavy chain variable region comprising an amino acid sequence having less than or equal to 7 total substitutions, deletions, and/or insertions in the amino acid sequence as set forth SEQ ID NO: 72, and a light chain variable region comprising an amino acid sequence having less than or equal to 7 total substitutions, deletions, and/or insertions in the amino acid sequence as set forth SEQ ID NO: 2. In some cases, the antibody provided herein comprises a heavy chain variable region comprising an amino acid sequence having less than or equal to 5 total substitutions, deletions, and/or insertions in the amino acid sequence as set forth SEQ ID NO: 72, and a light chain variable region comprising an amino acid sequence having less than or equal to 5 total substitutions, deletions, and/or insertions in the amino acid sequence as set forth SEQ ID NO: 2. In some cases, the antibody provided herein comprises a heavy chain variable region comprising an amino acid sequence having less than or equal to 3 total substitutions, deletions, and/or insertions in the amino acid sequence as set forth SEQ ID NO: 72, and a light chain variable region comprising an amino acid sequence having less than or equal to 3 total substitutions, deletions, and/or insertions in the amino acid sequence as set forth SEQ ID NO: 2. In some cases, the antibody provided herein comprises a heavy chain variable region comprising an amino acid sequence having less than or equal to 2 total substitutions, deletions, and/or insertions in the amino acid sequence as set forth SEQ ID NO: 72, and a light chain variable region comprising an amino acid sequence having less than or equal to 2 total substitutions, deletions, and/or insertions in the amino acid sequence as set forth SEQ ID NO: 2. In some cases, the antibody provided herein comprises a heavy chain variable region comprising an amino acid sequence having less than or equal to 1 total substitutions, deletions, and/or insertions in the amino acid sequence as set forth SEQ ID NO: 72, and a light chain variable region comprising an amino acid sequence having less than or equal to 1 total substitutions, deletions, and/or insertions in the amino acid sequence as set forth SEQ ID NO: 2.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 72, and a light chain variable region comprising an amino acid sequence having at least 90%, 95%, 96%, 97%, 98%, 99%, or 100% sequence identity to the amino acid sequence as set forth SEQ ID NO: 48. In some cases, the antibody provided herein comprises a heavy chain variable region comprising an amino acid sequence having at least 95% sequence identity to the amino acid sequence as set forth SEQ ID NO: 72, and a light chain variable region comprising an amino acid sequence having at least 95% sequence identity to the amino acid sequence as set forth SEQ ID NO: 48. In some cases, the antibody provided herein comprises a heavy chain variable region comprising an amino acid sequence having at least 97% sequence identity to the amino acid sequence as set forth SEQ ID NO: 72, and a light chain variable region comprising an amino acid sequence having at least 97% sequence identity to the amino acid sequence as set forth SEQ ID NO: 48. In some cases, the antibody provided herein comprises a heavy chain variable region comprising an amino acid sequence having at least 99% sequence identity to the amino acid sequence as set forth SEQ ID NO: 72, and a light chain variable region comprising an amino acid sequence having at least 99% sequence identity to the amino acid sequence as set forth SEQ ID NO: 48. In some cases, the antibody provided herein comprises a heavy chain variable region comprising an amino acid sequence having 100% sequence identity to the amino acid sequence as set forth SEQ ID NO: 72, and a light chain variable region comprising an amino acid sequence having 100% sequence identity to the amino acid sequence as set forth SEQ ID NO: 48. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 72, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 48. In some cases, the antibody provided herein comprises a heavy chain variable region comprising an amino acid sequence having less than or equal to 10 total substitutions, deletions, and/or insertions in the amino acid sequence as set forth SEQ ID NO: 72, and a light chain variable region comprising an amino acid sequence having less than or equal to 10 total substitutions, deletions, and/or insertions in the amino acid sequence as set forth SEQ ID NO: 48. In some cases, the antibody provided herein comprises a heavy chain variable region comprising an amino acid sequence having less than or equal to 7 total substitutions, deletions, and/or insertions in the amino acid sequence as set forth SEQ ID NO: 72, and a light chain variable region comprising an amino acid sequence having less than or equal to 7 total substitutions, deletions, and/or insertions in the amino acid sequence as set forth SEQ ID NO: 48. In some cases, the antibody provided herein comprises a heavy chain variable region comprising an amino acid sequence having less than or equal to 5 total substitutions, deletions, and/or insertions in the amino acid sequence as set forth SEQ ID NO: 72, and a light chain variable region comprising an amino acid sequence having less than or equal to 5 total substitutions, deletions, and/or insertions in the amino acid sequence as set forth SEQ ID NO: 48. In some cases, the antibody provided herein comprises a heavy chain variable region comprising an amino acid sequence having less than or equal to 3 total substitutions, deletions, and/or insertions in the amino acid sequence as set forth SEQ ID NO: 72, and a light chain variable region comprising an amino acid sequence having less than or equal to 3 total substitutions, deletions, and/or insertions in the amino acid sequence as set forth SEQ ID NO: 48. In some cases, the antibody provided herein comprises a heavy chain variable region comprising an amino acid sequence having less than or equal to 2 total substitutions, deletions, and/or insertions in the amino acid sequence as set forth SEQ ID NO: 72, and a light chain variable region comprising an amino acid sequence having less than or equal to 2 total substitutions, deletions, and/or insertions in the amino acid sequence as set forth SEQ ID NO: 48. In some cases, the antibody provided herein comprises a heavy chain variable region comprising an amino acid sequence having less than or equal to 1 total substitutions, deletions, and/or insertions in the amino acid sequence as set forth SEQ ID NO: 72, and a light chain variable region comprising an amino acid sequence having less than or equal to 1 total substitutions, deletions, and/or insertions in the amino acid sequence as set forth SEQ ID NO: 48

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 72, and a light chain variable region comprising an amino acid sequence having at least 90%, 95%, 96%, 97%, 98%, 99%, or 100% sequence identity to the amino acid sequence as set forth SEQ ID NO: 49. In some cases, the antibody provided herein comprises a heavy chain variable region comprising an amino acid sequence having at least 95% sequence identity to the amino acid sequence as set forth SEQ ID NO: 72, and a light chain variable region comprising an amino acid sequence having at least 95% sequence identity to the amino acid sequence as set forth SEQ ID NO: 49. In some cases, the antibody provided herein comprises a heavy chain variable region comprising an amino acid sequence having at least 97% sequence identity to the amino acid sequence as set forth SEQ ID NO: 72, and a light chain variable region comprising an amino acid sequence having at least 97% sequence identity to the amino acid sequence as set forth SEQ ID NO: 49. In some cases, the antibody provided herein comprises a heavy chain variable region comprising an amino acid sequence having at least 99% sequence identity to the amino acid sequence as set forth SEQ ID NO: 72, and a light chain variable region comprising an amino acid sequence having at least 99% sequence identity to the amino acid sequence as set forth SEQ ID NO: 49. In some cases, the antibody provided herein comprises a heavy chain variable region comprising an amino acid sequence having 100% sequence identity to the amino acid sequence as set forth SEQ ID NO: 72, and a light chain variable region comprising an amino acid sequence having 100% sequence identity to the amino acid sequence as set forth SEQ ID NO: 49. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 72, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 49. In some cases, the antibody provided herein comprises a heavy chain variable region comprising an amino acid sequence having less than or equal to 10 total substitutions, deletions, and/or insertions in the amino acid sequence as set forth SEQ ID NO: 72, and a light chain variable region comprising an amino acid sequence having less than or equal to 10 total substitutions, deletions, and/or insertions in the amino acid sequence as set forth SEQ ID NO: 49. In some cases, the antibody provided herein comprises a heavy chain variable region comprising an amino acid sequence having less than or equal to 7 total substitutions, deletions, and/or insertions in the amino acid sequence as set forth SEQ ID NO: 72, and a light chain variable region comprising an amino acid sequence having less than or equal to 7 total substitutions, deletions, and/or insertions in the amino acid sequence as set forth SEQ ID NO: 49. In some cases, the antibody provided herein comprises a heavy chain variable region comprising an amino acid sequence having less than or equal to 5 total substitutions, deletions, and/or insertions in the amino acid sequence as set forth SEQ ID NO: 72, and a light chain variable region comprising an amino acid sequence having less than or equal to 5 total substitutions, deletions, and/or insertions in the amino acid sequence as set forth SEQ ID NO: 49. In some cases, the antibody provided herein comprises a heavy chain variable region comprising an amino acid sequence having less than or equal to 3 total substitutions, deletions, and/or insertions in the amino acid sequence as set forth SEQ ID NO: 72, and a light chain variable region comprising an amino acid sequence having less than or equal to 3 total substitutions, deletions, and/or insertions in the amino acid sequence as set forth SEQ ID NO: 49. In some cases, the antibody provided herein comprises a heavy chain variable region comprising an amino acid sequence having less than or equal to 2 total substitutions, deletions, and/or insertions in the amino acid sequence as set forth SEQ ID NO: 72, and a light chain variable region comprising an amino acid sequence having less than or equal to 2 total substitutions, deletions, and/or insertions in the amino acid sequence as set forth SEQ ID NO: 49. In some cases, the antibody provided herein comprises a heavy chain variable region comprising an amino acid sequence having less than or equal to 1 total substitutions, deletions, and/or insertions in the amino acid sequence as set forth SEQ ID NO: 72, and a light chain variable region comprising an amino acid sequence having less than or equal to 1 total substitutions, deletions, and/or insertions in the amino acid sequence as set forth SEQ ID NO: 49.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 72, and a light chain variable region comprising an amino acid sequence having at least 90%, 95%, 96%, 97%, 98%, 99%, or 100% sequence identity to the amino acid sequence as set forth SEQ ID NO: 50. In some cases, the antibody provided herein comprises a heavy chain variable region comprising an amino acid sequence having at least 95% sequence identity to the amino acid sequence as set forth SEQ ID NO: 72, and a light chain variable region comprising an amino acid sequence having at least 95% sequence identity to the amino acid sequence as set forth SEQ ID NO: 50. In some cases, the antibody provided herein comprises a heavy chain variable region comprising an amino acid sequence having at least 97% sequence identity to the amino acid sequence as set forth SEQ ID NO: 72, and a light chain variable region comprising an amino acid sequence having at least 97% sequence identity to the amino acid sequence as set forth SEQ ID NO: 50. In some cases, the antibody provided herein comprises a heavy chain variable region comprising an amino acid sequence having at least 99% sequence identity to the amino acid sequence as set forth SEQ ID NO: 72, and a light chain variable region comprising an amino acid sequence having at least 99% sequence identity to the amino acid sequence as set forth SEQ ID NO: 50. In some cases, the antibody provided herein comprises a heavy chain variable region comprising an amino acid sequence having 100% sequence identity to the amino acid sequence as set forth SEQ ID NO: 72, and a light chain variable region comprising an amino acid sequence having 100% sequence identity to the amino acid sequence as set forth SEQ ID NO: 50. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 72, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 50. In some cases, the antibody provided herein comprises a heavy chain variable region comprising an amino acid sequence having less than or equal to 10 total substitutions, deletions, and/or insertions in the amino acid sequence as set forth SEQ ID NO: 72, and a light chain variable region comprising an amino acid sequence having less than or equal to 10 total substitutions, deletions, and/or insertions in the amino acid sequence as set forth SEQ ID NO: 50. In some cases, the antibody provided herein comprises a heavy chain variable region comprising an amino acid sequence having less than or equal to 7 total substitutions, deletions, and/or insertions in the amino acid sequence as set forth SEQ ID NO: 72, and a light chain variable region comprising an amino acid sequence having less than or equal to 7 total substitutions, deletions, and/or insertions in the amino acid sequence as set forth SEQ ID NO: 50. In some cases, the antibody provided herein comprises a heavy chain variable region comprising an amino acid sequence having less than or equal to 5 total substitutions, deletions, and/or insertions in the amino acid sequence as set forth SEQ ID NO: 72, and a light chain variable region comprising an amino acid sequence having less than or equal to 5 total substitutions, deletions, and/or insertions in the amino acid sequence as set forth SEQ ID NO: 50. In some cases, the antibody provided herein comprises a heavy chain variable region comprising an amino acid sequence having less than or equal to 3 total substitutions, deletions, and/or insertions in the amino acid sequence as set forth SEQ ID NO: 72, and a light chain variable region comprising an amino acid sequence having less than or equal to 3 total substitutions, deletions, and/or insertions in the amino acid sequence as set forth SEQ ID NO: 50. In some cases, the antibody provided herein comprises a heavy chain variable region comprising an amino acid sequence having less than or equal to 2 total substitutions, deletions, and/or insertions in the amino acid sequence as set forth SEQ ID NO: 72, and a light chain variable region comprising an amino acid sequence having less than or equal to 2 total substitutions, deletions, and/or insertions in the amino acid sequence as set forth SEQ ID NO: 50. In some cases, the antibody provided herein comprises a heavy chain variable region comprising an amino acid sequence having less than or equal to 1 total substitutions, deletions, and/or insertions in the amino acid sequence as set forth SEQ ID NO: 72, and a light chain variable region comprising an amino acid sequence having less than or equal to 1 total substitutions, deletions, and/or insertions in the amino acid sequence as set forth SEQ ID NO: 50.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 72, and a light chain variable region comprising an amino acid sequence having at least 90%, 95%, 96%, 97%, 98%, 99%, or 100% sequence identity to the amino acid sequence as set forth SEQ ID NO: 51. In some cases, the antibody provided herein comprises a heavy chain variable region comprising an amino acid sequence having at least 95% sequence identity to the amino acid sequence as set forth SEQ ID NO: 72, and a light chain variable region comprising an amino acid sequence having at least 95% sequence identity to the amino acid sequence as set forth SEQ ID NO: 51. In some cases, the antibody provided herein comprises a heavy chain variable region comprising an amino acid sequence having at least 97% sequence identity to the amino acid sequence as set forth SEQ ID NO: 72, and a light chain variable region comprising an amino acid sequence having at least 97% sequence identity to the amino acid sequence as set forth SEQ ID NO: 51. In some cases, the antibody provided herein comprises a heavy chain variable region comprising an amino acid sequence having at least 99% sequence identity to the amino acid sequence as set forth SEQ ID NO: 72, and a light chain variable region comprising an amino acid sequence having at least 99% sequence identity to the amino acid sequence as set forth SEQ ID NO: 51. In some cases, the antibody provided herein comprises a heavy chain variable region comprising an amino acid sequence having 100% sequence identity to the amino acid sequence as set forth SEQ ID NO: 72, and a light chain variable region comprising an amino acid sequence having 100% sequence identity to the amino acid sequence as set forth SEQ ID NO: 51. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 72, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 51. In some cases, the antibody provided herein comprises a heavy chain variable region comprising an amino acid sequence having less than or equal to 10 total substitutions, deletions, and/or insertions in the amino acid sequence as set forth SEQ ID NO: 72, and a light chain variable region comprising an amino acid sequence having less than or equal to 10 total substitutions, deletions, and/or insertions in the amino acid sequence as set forth SEQ ID NO: 51. In some cases, the antibody provided herein comprises a heavy chain variable region comprising an amino acid sequence having less than or equal to 7 total substitutions, deletions, and/or insertions in the amino acid sequence as set forth SEQ ID NO: 72, and a light chain variable region comprising an amino acid sequence having less than or equal to 7 total substitutions, deletions, and/or insertions in the amino acid sequence as set forth SEQ ID NO: 51. In some cases, the antibody provided herein comprises a heavy chain variable region comprising an amino acid sequence having less than or equal to 5 total substitutions, deletions, and/or insertions in the amino acid sequence as set forth SEQ ID NO: 72, and a light chain variable region comprising an amino acid sequence having less than or equal to 5 total substitutions, deletions, and/or insertions in the amino acid sequence as set forth SEQ ID NO: 51. In some cases, the antibody provided herein comprises a heavy chain variable region comprising an amino acid sequence having less than or equal to 3 total substitutions, deletions, and/or insertions in the amino acid sequence as set forth SEQ ID NO: 72, and a light chain variable region comprising an amino acid sequence having less than or equal to 3 total substitutions, deletions, and/or insertions in the amino acid sequence as set forth SEQ ID NO: 51. In some cases, the antibody provided herein comprises a heavy chain variable region comprising an amino acid sequence having less than or equal to 2 total substitutions, deletions, and/or insertions in the amino acid sequence as set forth SEQ ID NO: 72, and a light chain variable region comprising an amino acid sequence having less than or equal to 2 total substitutions, deletions, and/or insertions in the amino acid sequence as set forth SEQ ID NO: 51. In some cases, the antibody provided herein comprises a heavy chain variable region comprising an amino acid sequence having less than or equal to 1 total substitutions, deletions, and/or insertions in the amino acid sequence as set forth SEQ ID NO: 72, and a light chain variable region comprising an amino acid sequence having less than or equal to 1 total substitutions, deletions, and/or insertions in the amino acid sequence as set forth SEQ ID NO: 51

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 72, and a light chain variable region comprising an amino acid sequence having at least 90%, 95%, 96%, 97%, 98%, 99%, or 100% sequence identity to the amino acid sequence as set forth SEQ ID NO: 52. In some cases, the antibody provided herein comprises a heavy chain variable region comprising an amino acid sequence having at least 95% sequence identity to the amino acid sequence as set forth SEQ ID NO: 72, and a light chain variable region comprising an amino acid sequence having at least 95% sequence identity to the amino acid sequence as set forth SEQ ID NO: 52. In some cases, the antibody provided herein comprises a heavy chain variable region comprising an amino acid sequence having at least 97% sequence identity to the amino acid sequence as set forth SEQ ID NO: 72, and a light chain variable region comprising an amino acid sequence having at least 97% sequence identity to the amino acid sequence as set forth SEQ ID NO: 52. In some cases, the antibody provided herein comprises a heavy chain variable region comprising an amino acid sequence having at least 99% sequence identity to the amino acid sequence as set forth SEQ ID NO: 72, and a light chain variable region comprising an amino acid sequence having at least 99% sequence identity to the amino acid sequence as set forth SEQ ID NO: 52. In some cases, the antibody provided herein comprises a heavy chain variable region comprising an amino acid sequence having 100% sequence identity to the amino acid sequence as set forth SEQ ID NO: 72, and a light chain variable region comprising an amino acid sequence having 100% sequence identity to the amino acid sequence as set forth SEQ ID NO: 52. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 72, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 52. In some cases, the antibody provided herein comprises a heavy chain variable region comprising an amino acid sequence having less than or equal to 10 total substitutions, deletions, and/or insertions in the amino acid sequence as set forth SEQ ID NO: 72, and a light chain variable region comprising an amino acid sequence having less than or equal to 10 total substitutions, deletions, and/or insertions in the amino acid sequence as set forth SEQ ID NO: 52. In some cases, the antibody provided herein comprises a heavy chain variable region comprising an amino acid sequence having less than or equal to 7 total substitutions, deletions, and/or insertions in the amino acid sequence as set forth SEQ ID NO: 72, and a light chain variable region comprising an amino acid sequence having less than or equal to 7 total substitutions, deletions, and/or insertions in the amino acid sequence as set forth SEQ ID NO: 52. In some cases, the antibody provided herein comprises a heavy chain variable region comprising an amino acid sequence having less than or equal to 5 total substitutions, deletions, and/or insertions in the amino acid sequence as set forth SEQ ID NO: 72, and a light chain variable region comprising an amino acid sequence having less than or equal to 5 total substitutions, deletions, and/or insertions in the amino acid sequence as set forth SEQ ID NO: 52. In some cases, the antibody provided herein comprises a heavy chain variable region comprising an amino acid sequence having less than or equal to 3 total substitutions, deletions, and/or insertions in the amino acid sequence as set forth SEQ ID NO: 72, and a light chain variable region comprising an amino acid sequence having less than or equal to 3 total substitutions, deletions, and/or insertions in the amino acid sequence as set forth SEQ ID NO: 52. In some cases, the antibody provided herein comprises a heavy chain variable region comprising an amino acid sequence having less than or equal to 2 total substitutions, deletions, and/or insertions in the amino acid sequence as set forth SEQ ID NO: 72, and a light chain variable region comprising an amino acid sequence having less than or equal to 2 total substitutions, deletions, and/or insertions in the amino acid sequence as set forth SEQ ID NO: 52. In some cases, the antibody provided herein comprises a heavy chain variable region comprising an amino acid sequence having less than or equal to 1 total substitutions, deletions, and/or insertions in the amino acid sequence as set forth SEQ ID NO: 72, and a light chain variable region comprising an amino acid sequence having less than or equal to 1 total substitutions, deletions, and/or insertions in the amino acid sequence as set forth SEQ ID NO: 52.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 72, and a light chain variable region comprising an amino acid sequence having at least 90%, 95%, 96%, 97%, 98%, 99%, or 100% sequence identity to the amino acid sequence as set forth SEQ ID NO: 65. In some cases, the antibody provided herein comprises a heavy chain variable region comprising an amino acid sequence having at least 95% sequence identity to the amino acid sequence as set forth SEQ ID NO: 72, and a light chain variable region comprising an amino acid sequence having at least 95% sequence identity to the amino acid sequence as set forth SEQ ID NO: 65. In some cases, the antibody provided herein comprises a heavy chain variable region comprising an amino acid sequence having at least 97% sequence identity to the amino acid sequence as set forth SEQ ID NO: 72, and a light chain variable region comprising an amino acid sequence having at least 97% sequence identity to the amino acid sequence as set forth SEQ ID NO: 65. In some cases, the antibody provided herein comprises a heavy chain variable region comprising an amino acid sequence having at least 99% sequence identity to the amino acid sequence as set forth SEQ ID NO: 72, and a light chain variable region comprising an amino acid sequence having at least 99% sequence identity to the amino acid sequence as set forth SEQ ID NO: 65. In some cases, the antibody provided herein comprises a heavy chain variable region comprising an amino acid sequence having 100% sequence identity to the amino acid sequence as set forth SEQ ID NO: 72, and a light chain variable region comprising an amino acid sequence having 100% sequence identity to the amino acid sequence as set forth SEQ ID NO: 65. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 72, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 65. In some cases, the antibody provided herein comprises a heavy chain variable region comprising an amino acid sequence having less than or equal to 10 total substitutions, deletions, and/or insertions in the amino acid sequence as set forth SEQ ID NO: 72, and a light chain variable region comprising an amino acid sequence having less than or equal to 10 total substitutions, deletions, and/or insertions in the amino acid sequence as set forth SEQ ID NO: 65. In some cases, the antibody provided herein comprises a heavy chain variable region comprising an amino acid sequence having less than or equal to 7 total substitutions, deletions, and/or insertions in the amino acid sequence as set forth SEQ ID NO: 72, and a light chain variable region comprising an amino acid sequence having less than or equal to 7 total substitutions, deletions, and/or insertions in the amino acid sequence as set forth SEQ ID NO: 65. In some cases, the antibody provided herein comprises a heavy chain variable region comprising an amino acid sequence having less than or equal to 5 total substitutions, deletions, and/or insertions in the amino acid sequence as set forth SEQ ID NO: 72, and a light chain variable region comprising an amino acid sequence having less than or equal to 5 total substitutions, deletions, and/or insertions in the amino acid sequence as set forth SEQ ID NO: 65. In some cases, the antibody provided herein comprises a heavy chain variable region comprising an amino acid sequence having less than or equal to 3 total substitutions, deletions, and/or insertions in the amino acid sequence as set forth SEQ ID NO: 72, and a light chain variable region comprising an amino acid sequence having less than or equal to 3 total substitutions, deletions, and/or insertions in the amino acid sequence as set forth SEQ ID NO: 65. In some cases, the antibody provided herein comprises a heavy chain variable region comprising an amino acid sequence having less than or equal to 2 total substitutions, deletions, and/or insertions in the amino acid sequence as set forth SEQ ID NO: 72, and a light chain variable region comprising an amino acid sequence having less than or equal to 2 total substitutions, deletions, and/or insertions in the amino acid sequence as set forth SEQ ID NO: 65. In some cases, the antibody provided herein comprises a heavy chain variable region comprising an amino acid sequence having less than or equal to 1 total substitutions, deletions, and/or insertions in the amino acid sequence as set forth SEQ ID NO: 72, and a light chain variable region comprising an amino acid sequence having less than or equal to 1 total substitutions, deletions, and/or insertions in the amino acid sequence as set forth SEQ ID NO: 65.

In some cases, the antibody provided herein comprises a heavy chain variable region comprising an amino acid sequence having at least 90%, 95%, 96%, 97%, 98%, 99%, or 100% sequence identity to the amino acid sequence as set forth SEQ ID NO: 72, and a light chain variable region comprising an amino acid sequence having at least 90%, 95%, 96%, 97%, 98%, 99%, or 100% sequence identity to the amino acid sequence as set forth SEQ ID NO: 66. In some cases, the antibody provided herein comprises a heavy chain variable region comprising an amino acid sequence having at least 95% sequence identity to the amino acid sequence as set forth SEQ ID NO: 72, and a light chain variable region comprising an amino acid sequence having at least 95% sequence identity to the amino acid sequence as set forth SEQ ID NO: 66. In some cases, the antibody provided herein comprises a heavy chain variable region comprising an amino acid sequence having at least 97% sequence identity to the amino acid sequence as set forth SEQ ID NO: 72, and a light chain variable region comprising an amino acid sequence having at least 97% sequence identity to the amino acid sequence as set forth SEQ ID NO: 66. In some cases, the antibody provided herein comprises a heavy chain variable region comprising an amino acid sequence having at least 99% sequence identity to the amino acid sequence as set forth SEQ ID NO: 72, and a light chain variable region comprising an amino acid sequence having at least 99% sequence identity to the amino acid sequence as set forth SEQ ID NO: 66. In some cases, the antibody provided herein comprises a heavy chain variable region comprising an amino acid sequence having 100% sequence identity to the amino acid sequence as set forth SEQ ID NO: 72, and a light chain variable region comprising an amino acid sequence having 100% sequence identity to the amino acid sequence as set forth SEQ ID NO: 66. In some cases, the antibody provided herein comprises a heavy chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 72, and a light chain variable region comprising an amino acid sequence having less than or equal to 10, 9, 8, 7, 6, 5, 4, 3, 2, 1 or 0 total substitutions, deletions, and/or insertions in the amino acid sequence as set forth SEQ ID NO: 66. In some cases, the antibody provided herein comprises a heavy chain variable region comprising an amino acid sequence having less than or equal to 10 total substitutions, deletions, and/or insertions in the amino acid sequence as set forth SEQ ID NO: 72, and a light chain variable region comprising an amino acid sequence having less than or equal to 10 total substitutions, deletions, and/or insertions in the amino acid sequence as set forth SEQ ID NO: 66. In some cases, the antibody provided herein comprises a heavy chain variable region comprising an amino acid sequence having less than or equal to 7 total substitutions, deletions, and/or insertions in the amino acid sequence as set forth SEQ ID NO: 72, and a light chain variable region comprising an amino acid sequence having less than or equal to 7 total substitutions, deletions, and/or insertions in the amino acid sequence as set forth SEQ ID NO: 66. In some cases, the antibody provided herein comprises a heavy chain variable region comprising an amino acid sequence having less than or equal to 5 total substitutions, deletions, and/or insertions in the amino acid sequence as set forth SEQ ID NO: 72, and a light chain variable region comprising an amino acid sequence having less than or equal to 5 total substitutions, deletions, and/or insertions in the amino acid sequence as set forth SEQ ID NO: 66. In some cases, the antibody provided herein comprises a heavy chain variable region comprising an amino acid sequence having less than or equal to 3 total substitutions, deletions, and/or insertions in the amino acid sequence as set forth SEQ ID NO: 72, and a light chain variable region comprising an amino acid sequence having less than or equal to 3 total substitutions, deletions, and/or insertions in the amino acid sequence as set forth SEQ ID NO: 66. In some cases, the antibody provided herein comprises a heavy chain variable region comprising an amino acid sequence having less than or equal to 2 total substitutions, deletions, and/or insertions in the amino acid sequence as set forth SEQ ID NO: 72, and a light chain variable region comprising an amino acid sequence having less than or equal to 2 total substitutions, deletions, and/or insertions in the amino acid sequence as set forth SEQ ID NO: 66. In some cases, the antibody provided herein comprises a heavy chain variable region comprising an amino acid sequence having less than or equal to 1 total substitutions, deletions, and/or insertions in the amino acid sequence as set forth SEQ ID NO: 72, and a light chain variable region comprising an amino acid sequence having less than or equal to 1 total substitutions, deletions, and/or insertions in the amino acid sequence as set forth SEQ ID NO: 66.

In some embodiments, the antibody provided herein comprises a heavy chain variable region comprising an amino acid sequence as set forth in SEQ ID NO: 1, with from 0 to 3 amino acid modifications, and a light chain variable region, comprising an amino acid sequence as set forth in SEQ ID NO: 2, with from 0 to 3 amino acid modifications. In some cases, the antibody provided herein comprises a heavy chain variable region comprising an amino acid sequence as set forth in SEQ ID NO: 1, with from 0 to 2 amino acid modifications, and a light chain variable region, comprising an amino acid sequence as set forth in SEQ ID NO: 2, with from 0 to 2 amino acid modifications. In some cases, the antibody provided herein comprises a heavy chain variable region comprising an amino acid sequence as set forth in SEQ ID NO: 1, with from 0 to 1 amino acid modifications, and a light chain variable region, comprising an amino acid sequence as set forth in SEQ ID NO: 2, with from 0 to 1 amino acid modifications.

In some embodiments, the antibody provided herein comprises a heavy chain variable region comprising an amino acid sequence as set forth in SEQ ID NO: 9, with from 0 to 3 amino acid modifications, and a light chain variable region, comprising an amino acid sequence as set forth in SEQ ID NO: 10, with from 0 to 3 amino acid modifications. In some cases, the antibody provided herein comprises a heavy chain variable region comprising an amino acid sequence as set forth in SEQ ID NO: 9, with from 0 to 2 amino acid modifications, and a light chain variable region, comprising an amino acid sequence as set forth in SEQ ID NO: 10, with from 0 to 2 amino acid modifications. In some cases, the antibody provided herein comprises a heavy chain variable region comprising an amino acid sequence as set forth in SEQ ID NO: 9, with from 0 to 1 amino acid modifications, and a light chain variable region, comprising an amino acid sequence as set forth in SEQ ID NO: 10, with from 0 to 1 amino acid modifications.

In some embodiments, the antibody provided herein comprises a heavy chain variable region comprising an amino acid sequence as set forth in SEQ ID NO: 17, with from 0 to 3 amino acid modifications, and a light chain variable region, comprising an amino acid sequence as set forth in SEQ ID NO: 18, with from 0 to 3 amino acid modifications. In some cases, the antibody provided herein comprises a heavy chain variable region comprising an amino acid sequence as set forth in SEQ ID NO: 17, with from 0 to 2 amino acid modifications, and a light chain variable region, comprising an amino acid sequence as set forth in SEQ ID NO: 18, with from 0 to 2 amino acid modifications. In some cases, the antibody provided herein comprises a heavy chain variable region comprising an amino acid sequence as set forth in SEQ ID NO: 17, with from 0 to 1 amino acid modifications, and a light chain variable region, comprising an amino acid sequence as set forth in SEQ ID NO: 18, with from 0 to 1 amino acid modifications.

In some embodiments, the antibody provided herein comprises a heavy chain variable region comprising an amino acid sequence as set forth in SEQ ID NO: 25, with from 0 to 3 amino acid modifications, and a light chain variable region, comprising an amino acid sequence as set forth in SEQ ID NO: 26, with from 0 to 3 amino acid modifications. In some cases, the antibody provided herein comprises a heavy chain variable region comprising an amino acid sequence as set forth in SEQ ID NO: 25, with from 0 to 2 amino acid modifications, and a light chain variable region, comprising an amino acid sequence as set forth in SEQ ID NO: 26, with from 0 to 2 amino acid modifications. In some cases, the antibody provided herein comprises a heavy chain variable region comprising an amino acid sequence as set forth in SEQ ID NO: 25, with from 0 to 1 amino acid modifications, and a light chain variable region, comprising an amino acid sequence as set forth in SEQ ID NO: 26, with from 0 to 1 amino acid modifications.

In some embodiments, the antibody provided herein comprises a heavy chain variable region comprising an amino acid sequence as set forth in SEQ ID NO: 33, with from 0 to 3 amino acid modifications, and a light chain variable region, comprising an amino acid sequence as set forth in SEQ ID NO: 34, with from 0 to 3 amino acid modifications. In some cases, the antibody provided herein comprises a heavy chain variable region comprising an amino acid sequence as set forth in SEQ ID NO: 33, with from 0 to 2 amino acid modifications, and a light chain variable region, comprising an amino acid sequence as set forth in SEQ ID NO: 34, with from 0 to 2 amino acid modifications. In some cases, the antibody provided herein comprises a heavy chain variable region comprising an amino acid sequence as set forth in SEQ ID NO: 33, with from 0 to 1 amino acid modifications, and a light chain variable region, comprising an amino acid sequence as set forth in SEQ ID NO: 34, with from 0 to 1 amino acid modifications.

In some embodiments, the antibody provided herein comprises a heavy chain variable region comprising an amino acid sequence as set forth in SEQ ID NO: 71, with from 0 to 3 amino acid modifications, and a light chain variable region, comprising an amino acid sequence as set forth in SEQ ID NO: 65, with from 0 to 3 amino acid modifications. In some cases, the antibody provided herein comprises a heavy chain variable region comprising an amino acid sequence as set forth in SEQ ID NO: 71, with from 0 to 2 amino acid modifications, and a light chain variable region, comprising an amino acid sequence as set forth in SEQ ID NO: 65, with from 0 to 2 amino acid modifications. In some cases, the antibody provided herein comprises a heavy chain variable region comprising an amino acid sequence as set forth in SEQ ID NO: 71, with from 0 to 1 amino acid modifications, and a light chain variable region, comprising an amino acid sequence as set forth in SEQ ID NO: 65, with from 0 to 1 amino acid modifications.

In some embodiments, the antibody provided herein comprises a heavy chain variable region comprising an amino acid sequence as set forth in SEQ ID NO: 72, with from 0 to 3 amino acid modifications, and a light chain variable region, comprising an amino acid sequence as set forth in SEQ ID NO: 65, with from 0 to 3 amino acid modifications. In some cases, the antibody provided herein comprises a heavy chain variable region comprising an amino acid sequence as set forth in SEQ ID NO: 72, with from 0 to 2 amino acid modifications, and a light chain variable region, comprising an amino acid sequence as set forth in SEQ ID NO: 65, with from 0 to 2 amino acid modifications. In some cases, the antibody provided herein comprises a heavy chain variable region comprising an amino acid sequence as set forth in SEQ ID NO: 72, with from 0 to 1 amino acid modifications, and a light chain variable region, comprising an amino acid sequence as set forth in SEQ ID NO: 65, with from 0 to 1 amino acid modifications.

In some embodiments, the antibody provided herein comprises a heavy chain variable region comprising an amino acid sequence as set forth in any one of SEQ ID NOs: 1, 42, 43, 44, 45, 46, 47, 71, 72, or 93 with from 0 to 3 amino acid modifications, and a light chain variable region, comprising an amino acid sequence as set forth in any one of SEQ ID NOs: 2, 48, 49, 50, 51, 52, 65, 66, or 94, with from 0 to 3 amino acid modifications. In some cases, the antibody provided herein comprises a heavy chain variable region comprising an amino acid sequence as set forth in any one of SEQ ID NOs: 1, 42, 43, 44, 45, 46, 47, 71, 72, or 93 with from 0 to 2 amino acid modifications, and a light chain variable region, comprising an amino acid sequence as set forth in any one of SEQ ID NOs: 2, 48, 49, 50, 51, 52, 65, 66, or 94, with from 0 to 2 amino acid modifications. In some cases, the antibody provided herein comprises a heavy chain variable region comprising an amino acid sequence as set forth in any one of SEQ ID NOs: 1, 42, 43, 44, 45, 46, 47, 71, 72, or 93 with from 0 to 1 amino acid modifications, and a light chain variable region, comprising an amino acid sequence as set forth in any one of SEQ ID NOs: 2, 48, 49, 50, 51, 52, 65, 66, or 94, with from 0 to 1 amino acid modifications.

In some embodiments, the antibody provided herein comprises a heavy chain variable region comprising an amino acid sequence as set forth in any one of SEQ ID NOs: 42, 43, 44, 45, 46, 47, 71, 72, or 93 with from 0 to 3 amino acid modifications, and a light chain variable region, comprising an amino acid sequence as set forth in any one of SEQ ID NOs: 48, 49, 50, 51, 52, 65, 66, or 94 with from 0 to 3 amino acid modifications. In some cases, the antibody provided herein comprises a heavy chain variable region comprising an amino acid sequence as set forth in any one of SEQ ID NOs: 42, 43, 44, 45, 46, 47, 71, 72, or 93 with from 0 to 2 amino acid modifications, and a light chain variable region, comprising an amino acid sequence as set forth in any one of SEQ ID NOs: 48, 49, 50, 51, 52, 65, 66, or 94 with from 0 to 2 amino acid modifications. In some cases, the antibody provided herein comprises a heavy chain variable region comprising an amino acid sequence as set forth in any one of SEQ ID NOs: 42, 43, 44, 45, 46, 47, 71, 72, or 93 with from 0 to 1 amino acid modifications, and a light chain variable region, comprising an amino acid sequence as set forth in any one of SEQ ID NOs: 48, 49, 50, 51, 52, 65, 66, or 94 with from 0 to 1 amino acid modifications.

In some embodiments, the antibody provided herein that specifically binds CD200R comprises a heavy chain comprising a heavy chain variable region the further comprises an amino acid sequence having at least 90% sequence identity to the amino acid sequence as set forth in SEQ ID NO: 1, 9, 17, 25, 33, 42, 43, 44, 45, 46, 47, 71, 72, or 93. In some embodiments, the antibody provided herein that specifically binds CD200R comprises a heavy chain comprising a heavy chain variable region the further comprises an amino acid sequence having at least 95% sequence identity to the amino acid sequence as set forth in SEQ ID NO: 1, 9, 17, 25, 33, 42, 43, 44, 45, 46, 47, 71, 72, or 93. In some embodiments, the antibody provided herein that specifically binds CD200R comprises a heavy chain comprising a heavy chain variable region the further comprises an amino acid sequence having at least 96% sequence identity to the amino acid sequence as set forth in SEQ ID NO: 1, 9, 17, 25, 33, 42, 43, 44, 45, 46, 47, 71, 72, or 93. In some embodiments, the antibody provided herein that specifically binds CD200R comprises a heavy chain comprising a heavy chain variable region the further comprises an amino acid sequence having at least 97% sequence identity to the amino acid sequence as set forth in SEQ ID NO: 1, 9, 17, 25, 33, 42, 43, 44, 45, 46, 47, 71, 72, or 93. In some embodiments, the antibody provided herein that specifically binds CD200R comprises a heavy chain comprising a heavy chain variable region the further comprises an amino acid sequence having at least 98% sequence identity to the amino acid sequence as set forth in SEQ ID NO: 1, 9, 17, 25, 33, 42, 43, 44, 45, 46, 47, 71, 72, or 93. In some embodiments, the antibody provided herein that specifically binds CD200R comprises a heavy chain comprising a heavy chain variable region the further comprises an amino acid sequence having at least 99% sequence identity to the amino acid sequence as set forth in SEQ ID NO: 1, 9, 17, 25, 33, 42, 43, 44, 45, 46, 47, 71, 72, or 93. In some embodiments, the antibody provided herein that specifically binds CD200R comprises a heavy chain comprising a heavy chain variable region the further comprises an amino acid sequence having 100% sequence identity to the amino acid sequence as set forth in SEQ ID NO: 1, 9, 17, 25, 33, 42, 43, 44, 45, 46, 47, 71, 72, or 93.

In some embodiments, the antibody provided herein that specifically binds CD200R comprises a heavy chain variable region comprising an amino acid sequence as set forth in SEQ ID NO: 1, 9, 17, 25, 33, 42, 43, 44, 45, 46, 47, 71, 72, or 93with from 0 to 3 amino acid modifications.

In some cases, the antibody provided herein that specifically binds CD200R comprises a heavy chain variable region comprising an amino acid sequence as set forth in SEQ ID NO: 1, 9, 17, 25, 33, 42, 43, 44, 45, 46, 47, 71, 72, or 93with from 0 to 2 amino acid modifications. In some cases, the antibody provided herein that specifically binds CD200R comprises a heavy chain variable region comprising an amino acid sequence as set forth in SEQ ID NO: 1, 9, 17, 25, 33, 42, 43, 44, 45, 46, 47, 71, 72, or 93with from 0 to 1 amino acid modifications.

In some embodiments, the antibody provided herein that specifically binds CD200R comprises a light chain comprising a light chain variable region the further comprises an amino acid sequence having at least 90% sequence identity to the amino acid sequence as set forth in SEQ ID NO: 2, 10, 18, 26, 34, 48, 49, 50, 51, 52, 65, 66, or 94. In some cases, the antibody provided herein that specifically binds CD200R comprises a light chain comprising a light chain variable region the further comprises an amino acid sequence having at least 95% sequence identity to the amino acid sequence as set forth in SEQ ID NO: 2, 10, 18, 26, 34, 48, 49, 50, 51, 52, 65, 66, or 94. In some cases, the antibody provided herein that specifically binds CD200R comprises a light chain comprising a light chain variable region the further comprises an amino acid sequence having at least 96% sequence identity to the amino acid sequence as set forth in SEQ ID NO: 2, 10, 18, 26, 34, 48, 49, 50, 51, 52, 65, 66, or 94. In some cases, the antibody provided herein that specifically binds CD200R comprises a light chain comprising a light chain variable region the further comprises an amino acid sequence having at least 97% sequence identity to the amino acid sequence as set forth in SEQ ID NO: 2, 10, 18, 26, 34, 48, 49, 50, 51, 52, 65, 66, or 94. In some cases, the antibody provided herein that specifically binds CD200R comprises a light chain comprising a light chain variable region the further comprises an amino acid sequence having at least 98% sequence identity to the amino acid sequence as set forth in SEQ ID NO: 2, 10, 18, 26, 34, 48, 49, 50, 51, 52, 65, 66, or 94. In some cases, the antibody provided herein that specifically binds CD200R comprises a light chain comprising a light chain variable region the further comprises an amino acid sequence having at least 99% sequence identity to the amino acid sequence as set forth in SEQ ID NO: 2, 10, 18, 26, 34, 48, 49, 50, 51, 52, 65, 66, or 94. In some cases, the antibody provided herein that specifically binds CD200R comprises a light chain comprising a light chain variable region the further comprises an amino acid sequence having 100% sequence identity to the amino acid sequence as set forth in SEQ ID NO: 2, 10, 18, 26, 34, 48, 49, 50, 51, 52, 65, 66, or 94.

In some embodiments, the antibody provided herein that specifically binds CD200R comprises a light chain variable region comprising an amino acid sequence as set forth in SEQ ID NO: 2, 10, 18, 26, 34, 48, 49, 50, 51, 52, 65, 66, or 94, with from 0 to 3 amino acid modifications. In some cases, the antibody provided herein that specifically binds CD200R comprises a light chain variable region comprising an amino acid sequence as set forth in SEQ ID NO: 2, 10, 18, 26, 34, 48, 49, 50, 51, 52, 65, 66, or 94, with from 0 to 2 amino acid modifications. In some cases, the antibody provided herein that specifically binds CD200R comprises a light chain variable region comprising an amino acid sequence as set forth in SEQ ID NO: 2, 10, 18, 26, 34, 48, 49, 50, 51, 52, 65, 66, or 94, with from 0 to 1 amino acid modifications.

In some embodiments, the antibody provided herein that specifically binds CD200R comprises a heavy chain comprising a heavy chain variable region and a light chain comprising a light chain variable region. In some cases, the heavy chain variable region comprises an amino acid sequence having at least 90% sequence identity to the amino acid sequence as set forth in SEQ ID NO: 1, and the light chain variable region comprises an amino acid sequence having at least 90% sequence identity to the amino acid sequence as set forth in SEQ ID NO: 2. In some cases, the heavy chain variable region comprises an amino acid sequence having at least 95% sequence identity to the amino acid sequence as set forth in SEQ ID NO: 1, and the light chain variable region comprises an amino acid sequence having at least 95% sequence identity to the amino acid sequence as set forth in SEQ ID NO: 2. In some cases, the heavy chain variable region comprises an amino acid sequence having at least 96% sequence identity to the amino acid sequence as set forth in SEQ ID NO: 1, and the light chain variable region comprises an amino acid sequence having at least 96% sequence identity to the amino acid sequence as set forth in SEQ ID NO: 2. In some cases, the heavy chain variable region comprises an amino acid sequence having at least 97% sequence identity to the amino acid sequence as set forth in SEQ ID NO: 1, and the light chain variable region comprises an amino acid sequence having at least 97% sequence identity to the amino acid sequence as set forth in SEQ ID NO: 2. In some cases, the heavy chain variable region comprises an amino acid sequence having at least 97% sequence identity to the amino acid sequence as set forth in SEQ ID NO: 1, and the light chain variable region comprises an amino acid sequence having at least 97% sequence identity to the amino acid sequence as set forth in SEQ ID NO: 2. In some cases, the heavy chain variable region comprises an amino acid sequence having at least 98% sequence identity to the amino acid sequence as set forth in SEQ ID NO: 1, and the light chain variable region comprises an amino acid sequence having at least 98% sequence identity to the amino acid sequence as set forth in SEQ ID NO: 2. In some cases, the heavy chain variable region comprises an amino acid sequence having at least 99% sequence identity to the amino acid sequence as set forth in SEQ ID NO: 1, and the light chain variable region comprises an amino acid sequence having at least 99% sequence identity to the amino acid sequence as set forth in SEQ ID NO: 2. In some cases, the heavy chain variable region comprises an amino acid sequence having 100% sequence identity to the amino acid sequence as set forth in SEQ ID NO: 1, and the light chain variable region comprises an amino acid sequence having at least 100% sequence identity to the amino acid sequence as set forth in SEQ ID NO: 2.

In some cases, the heavy chain variable region comprises an amino acid sequence having at least 90% sequence identity to the amino acid sequence as set forth in SEQ ID NO: 9, and the light chain variable region comprises an amino acid sequence having at least 90% sequence identity to the amino acid sequence as set forth in SEQ ID NO: 10. In some cases, the heavy chain variable region comprises an amino acid sequence having at least 95% sequence identity to the amino acid sequence as set forth in SEQ ID NO: 9, and the light chain variable region comprises an amino acid sequence having at least 95% sequence identity to the amino acid sequence as set forth in SEQ ID NO: 10. In some cases, the heavy chain variable region comprises an amino acid sequence having at least 96% sequence identity to the amino acid sequence as set forth in SEQ ID NO: 9, and the light chain variable region comprises an amino acid sequence having at least 96% sequence identity to the amino acid sequence as set forth in SEQ ID NO: 10. In some cases, the heavy chain variable region comprises an amino acid sequence having at least 97% sequence identity to the amino acid sequence as set forth in SEQ ID NO: 9, and the light chain variable region comprises an amino acid sequence having at least 97% sequence identity to the amino acid sequence as set forth in SEQ ID NO: 10. In some cases, the heavy chain variable region comprises an amino acid sequence having at least 98% sequence identity to the amino acid sequence as set forth in SEQ ID NO: 9, and the light chain variable region comprises an amino acid sequence having at least 98% sequence identity to the amino acid sequence as set forth in SEQ ID NO: 10. In some cases, the heavy chain variable region comprises an amino acid sequence having at least 99% sequence identity to the amino acid sequence as set forth in SEQ ID NO: 9, and the light chain variable region comprises an amino acid sequence having at least 99% sequence identity to the amino acid sequence as set forth in SEQ ID NO: 10. In some cases, the heavy chain variable region comprises an amino acid sequence having 100% sequence identity to the amino acid sequence as set forth in SEQ ID NO: 9, and the light chain variable region comprises an amino acid sequence having 100% sequence identity to the amino acid sequence as set forth in SEQ ID NO: 10.

In some cases, the heavy chain variable region comprises an amino acid sequence having at least 90% sequence identity to the amino acid sequence as set forth in SEQ ID NO: 17, and the light chain variable region comprises an amino acid sequence having at least 90% sequence identity to the amino acid sequence as set forth in SEQ ID NO: 18. In some cases, the heavy chain variable region comprises an amino acid sequence having at least 95% sequence identity to the amino acid sequence as set forth in SEQ ID NO: 17, and the light chain variable region comprises an amino acid sequence having at least 95% sequence identity to the amino acid sequence as set forth in SEQ ID NO: 18. In some cases, the heavy chain variable region comprises an amino acid sequence having at least 96% sequence identity to the amino acid sequence as set forth in SEQ ID NO: 17, and the light chain variable region comprises an amino acid sequence having at least 96% sequence identity to the amino acid sequence as set forth in SEQ ID NO: 18. In some cases, the heavy chain variable region comprises an amino acid sequence having at least 97% sequence identity to the amino acid sequence as set forth in SEQ ID NO: 17, and the light chain variable region comprises an amino acid sequence having at least 97% sequence identity to the amino acid sequence as set forth in SEQ ID NO: 18. In some cases, the heavy chain variable region comprises an amino acid sequence having at least 98% sequence identity to the amino acid sequence as set forth in SEQ ID NO: 17, and the light chain variable region comprises an amino acid sequence having at least 98% sequence identity to the amino acid sequence as set forth in SEQ ID NO: 18. In some cases, the heavy chain variable region comprises an amino acid sequence having at least 99% sequence identity to the amino acid sequence as set forth in SEQ ID NO: 17, and the light chain variable region comprises an amino acid sequence having at least 99% sequence identity to the amino acid sequence as set forth in SEQ ID NO: 18. In some cases, the heavy chain variable region comprises an amino acid sequence having 100% sequence identity to the amino acid sequence as set forth in SEQ ID NO: 17, and the light chain variable region comprises an amino acid sequence having 100% sequence identity to the amino acid sequence as set forth in SEQ ID NO: 18.

In some cases, the heavy chain variable region comprises an amino acid sequence having at least 90% sequence identity to the amino acid sequence as set forth in SEQ ID NO:25, and the light chain variable region comprises an amino acid sequence having at least 90% sequence identity to the amino acid sequence as set forth in SEQ ID NO: 26. In some cases, the heavy chain variable region comprises an amino acid sequence having at least 95% sequence identity to the amino acid sequence as set forth in SEQ ID NO:25, and the light chain variable region comprises an amino acid sequence having at least 95% sequence identity to the amino acid sequence as set forth in SEQ ID NO: 26. In some cases, the heavy chain variable region comprises an amino acid sequence having at least 96% sequence identity to the amino acid sequence as set forth in SEQ ID NO:25, and the light chain variable region comprises an amino acid sequence having at least 96% sequence identity to the amino acid sequence as set forth in SEQ ID NO: 26. In some cases, the heavy chain variable region comprises an amino acid sequence having at least 97% sequence identity to the amino acid sequence as set forth in SEQ ID NO:25, and the light chain variable region comprises an amino acid sequence having at least 97% sequence identity to the amino acid sequence as set forth in SEQ ID NO: 26. In some cases, the heavy chain variable region comprises an amino acid sequence having at least 98% sequence identity to the amino acid sequence as set forth in SEQ ID NO:25, and the light chain variable region comprises an amino acid sequence having at least 98% sequence identity to the amino acid sequence as set forth in SEQ ID NO: 26. In some cases, the heavy chain variable region comprises an amino acid sequence having at least 99% sequence identity to the amino acid sequence as set forth in SEQ ID NO:25, and the light chain variable region comprises an amino acid sequence having at least 99% sequence identity to the amino acid sequence as set forth in SEQ ID NO: 26. In some cases, the heavy chain variable region comprises an amino acid sequence having 100% sequence identity to the amino acid sequence as set forth in SEQ ID NO:25, and the light chain variable region comprises an amino acid sequence having 100% sequence identity to the amino acid sequence as set forth in SEQ ID NO: 26.

In some cases, the heavy chain variable region comprises an amino acid sequence having at least 90% sequence identity to the amino acid sequence as set forth in SEQ ID NO: 33, and the light chain variable region comprises an amino acid sequence having at least 90% sequence identity to the amino acid sequence as set forth in SEQ ID NO: 34. In some cases, the heavy chain variable region comprises an amino acid sequence having at least 95% sequence identity to the amino acid sequence as set forth in SEQ ID NO: 33, and the light chain variable region comprises an amino acid sequence having at least 95% sequence identity to the amino acid sequence as set forth in SEQ ID NO: 34. In some cases, the heavy chain variable region comprises an amino acid sequence having at least 96% sequence identity to the amino acid sequence as set forth in SEQ ID NO: 33, and the light chain variable region comprises an amino acid sequence having at least 96% sequence identity to the amino acid sequence as set forth in SEQ ID NO: 34. In some cases, the heavy chain variable region comprises an amino acid sequence having at least 97% sequence identity to the amino acid sequence as set forth in SEQ ID NO: 33, and the light chain variable region comprises an amino acid sequence having at least 97% sequence identity to the amino acid sequence as set forth in SEQ ID NO: 34. In some cases, the heavy chain variable region comprises an amino acid sequence having at least 98% sequence identity to the amino acid sequence as set forth in SEQ ID NO: 33, and the light chain variable region comprises an amino acid sequence having at least 98% sequence identity to the amino acid sequence as set forth in SEQ ID NO: 34. In some cases, the heavy chain variable region comprises an amino acid sequence having at least 99% sequence identity to the amino acid sequence as set forth in SEQ ID NO: 33, and the light chain variable region comprises an amino acid sequence having at least 99% sequence identity to the amino acid sequence as set forth in SEQ ID NO: 34. In some cases, the heavy chain variable region comprises an amino acid sequence having 100% sequence identity to the amino acid sequence as set forth in SEQ ID NO: 33, and the light chain variable region comprises an amino acid sequence having 100% sequence identity to the amino acid sequence as set forth in SEQ ID NO: 34.

In some cases, the heavy chain variable region comprises an amino acid sequence having at least 90% sequence identity to the amino acid sequence as set forth in SEQ ID NO: 71, and the light chain variable region comprises an amino acid sequence having at least 90% sequence identity to the amino acid sequence as set forth in SEQ ID NO: 65. In some cases, the heavy chain variable region comprises an amino acid sequence having at least 95% sequence identity to the amino acid sequence as set forth in SEQ ID NO: 71, and the light chain variable region comprises an amino acid sequence having at least 95% sequence identity to the amino acid sequence as set forth in SEQ ID NO: 65. In some cases, the heavy chain variable region comprises an amino acid sequence having at least 96% sequence identity to the amino acid sequence as set forth in SEQ ID NO: 71, and the light chain variable region comprises an amino acid sequence having at least 96% sequence identity to the amino acid sequence as set forth in SEQ ID NO: 65. In some cases, the heavy chain variable region comprises an amino acid sequence having at least 97% sequence identity to the amino acid sequence as set forth in SEQ ID NO: 71, and the light chain variable region comprises an amino acid sequence having at least 97% sequence identity to the amino acid sequence as set forth in SEQ ID NO: 65. In some cases, the heavy chain variable region comprises an amino acid sequence having at least 98% sequence identity to the amino acid sequence as set forth in SEQ ID NO: 71, and the light chain variable region comprises an amino acid sequence having at least 98% sequence identity to the amino acid sequence as set forth in SEQ ID NO: 65. In some cases, the heavy chain variable region comprises an amino acid sequence having at least 99% sequence identity to the amino acid sequence as set forth in SEQ ID NO: 71, and the light chain variable region comprises an amino acid sequence having at least 99% sequence identity to the amino acid sequence as set forth in SEQ ID NO: 65. In some cases, the heavy chain variable region comprises an amino acid sequence having 100% sequence identity to the amino acid sequence as set forth in SEQ ID NO: 71, and the light chain variable region comprises an amino acid sequence having 100% sequence identity to the amino acid sequence as set forth in SEQ ID NO: 65.

In some cases, the heavy chain variable region comprises an amino acid sequence having at least 90% sequence identity to the amino acid sequence as set forth in SEQ ID NO: 72, and the light chain variable region comprises an amino acid sequence having at least 90% sequence identity to the amino acid sequence as set forth in SEQ ID NO: 65. In some cases, the heavy chain variable region comprises an amino acid sequence having at least 95% sequence identity to the amino acid sequence as set forth in SEQ ID NO: 72, and the light chain variable region comprises an amino acid sequence having at least 95% sequence identity to the amino acid sequence as set forth in SEQ ID NO: 65. In some cases, the heavy chain variable region comprises an amino acid sequence having at least 96% sequence identity to the amino acid sequence as set forth in SEQ ID NO: 72, and the light chain variable region comprises an amino acid sequence having at least 96% sequence identity to the amino acid sequence as set forth in SEQ ID NO: 65. In some cases, the heavy chain variable region comprises an amino acid sequence having at least 97% sequence identity to the amino acid sequence as set forth in SEQ ID NO: 72, and the light chain variable region comprises an amino acid sequence having at least 97% sequence identity to the amino acid sequence as set forth in SEQ ID NO: 65. In some cases, the heavy chain variable region comprises an amino acid sequence having at least 98% sequence identity to the amino acid sequence as set forth in SEQ ID NO: 72, and the light chain variable region comprises an amino acid sequence having at least 98% sequence identity to the amino acid sequence as set forth in SEQ ID NO: 65. In some cases, the heavy chain variable region comprises an amino acid sequence having at least 99% sequence identity to the amino acid sequence as set forth in SEQ ID NO: 72, and the light chain variable region comprises an amino acid sequence having at least 99% sequence identity to the amino acid sequence as set forth in SEQ ID NO: 65. In some cases, the heavy chain variable region comprises an amino acid sequence having 100% sequence identity to the amino acid sequence as set forth in SEQ ID NO: 72, and the light chain variable region comprises an amino acid sequence having 100% sequence identity to the amino acid sequence as set forth in SEQ ID NO: 65.

In some cases, the heavy chain variable region comprises an amino acid sequence having at least 90% sequence identity to the amino acid sequence as set forth in SEQ ID NOs: 1, 42, 43, 44, 45, 46, 47, 71, 72, or 93 and the light chain variable region comprises an amino acid sequence having at least 90% sequence identity to the amino acid sequence as set forth in SEQ ID NO: 2, 48, 49, 50, 51, 52, 65, 66, or 94. In some cases, the heavy chain variable region comprises an amino acid sequence having at least 95% sequence identity to the amino acid sequence as set forth in SEQ ID NOs: 1, 42, 43, 44, 45, 46, 47, 71, 72, or 93 and the light chain variable region comprises an amino acid sequence having at least 95% sequence identity to the amino acid sequence as set forth in SEQ ID NO: 2, 48, 49, 50, 51, 52, 65, 66, or 94. In some cases, the heavy chain variable region comprises an amino acid sequence having at least 96% sequence identity to the amino acid sequence as set forth in SEQ ID NOs: 1, 42, 43, 44, 45, 46, 47, 71, 72, or 93 and the light chain variable region comprises an amino acid sequence having at least 96% sequence identity to the amino acid sequence as set forth in SEQ ID NO: 2, 48, 49, 50, 51, 52, 65, 66, or 94. In some cases, the heavy chain variable region comprises an amino acid sequence having at least 97% sequence identity to the amino acid sequence as set forth in SEQ ID NOs: 1, 42, 43, 44, 45, 46, 47, 71, 72, or 93 and the light chain variable region comprises an amino acid sequence having at least 97% sequence identity to the amino acid sequence as set forth in SEQ ID NO: 2, 48, 49, 50, 51, 52, 65, 66, or 94. In some cases, the heavy chain variable region comprises an amino acid sequence having at least 98% sequence identity to the amino acid sequence as set forth in SEQ ID NOs: 1, 42, 43, 44, 45, 46, 47, 71, 72, or 93 and the light chain variable region comprises an amino acid sequence having at least 98% sequence identity to the amino acid sequence as set forth in SEQ ID NO: 2, 48, 49, 50, 51, 52, 65, 66, or 94. In some cases, the heavy chain variable region comprises an amino acid sequence having at least 99% sequence identity to the amino acid sequence as set forth in SEQ ID NOs: 1, 42, 43, 44, 45, 46, 47, 71, 72, or 93 and the light chain variable region comprises an amino acid sequence having at least 99% sequence identity to the amino acid sequence as set forth in SEQ ID NO: 2, 48, 49, 50, 51, 52, 65, 66, or 94. In some cases, the heavy chain variable region comprises an amino acid sequence having 100% sequence identity to the amino acid sequence as set forth in SEQ ID NOs: 1, 42, 43, 44, 45, 46, 47, 71, 72, or 93 and the light chain variable region comprises an amino acid sequence having 100% sequence identity to the amino acid sequence as set forth in SEQ ID NO: 2, 48, 49, 50, 51, 52, 65, 66, or 94.

In some cases, the heavy chain variable region comprises an amino acid sequence having at least 90% sequence identity to the amino acid sequence as set forth in SEQ ID NOs: 42, 43, 44, 45, 46, 47, 71, 72, or 93 and the light chain variable region comprises an amino acid sequence having at least 90% sequence identity to the amino acid sequence as set forth in SEQ ID NOS: 48, 49, 50, 51, 52, 65, 66, or 94. In some cases, the heavy chain variable region comprises an amino acid sequence having at least 95% sequence identity to the amino acid sequence as set forth in SEQ ID NOs: 42, 43, 44, 45, 46, 47, 71, 72, or 93 and the light chain variable region comprises an amino acid sequence having at least 95% sequence identity to the amino acid sequence as set forth in SEQ ID NOS: 48, 49, 50, 51, 52, 65, 66, or 94. In some cases, the heavy chain variable region comprises an amino acid sequence having at least 96% sequence identity to the amino acid sequence as set forth in SEQ ID NOs: 42, 43, 44, 45, 46, 47, 71, 72, or 93 and the light chain variable region comprises an amino acid sequence having at least 96% sequence identity to the amino acid sequence as set forth in SEQ ID NOS: 48, 49, 50, 51, 52, 65, 66, or 94. In some cases, the heavy chain variable region comprises an amino acid sequence having at least 97% sequence identity to the amino acid sequence as set forth in SEQ ID NOs: 42, 43, 44, 45, 46, 47, 71, 72, or 93 and the light chain variable region comprises an amino acid sequence having at least 97% sequence identity to the amino acid sequence as set forth in SEQ ID NOS: 48, 49, 50, 51, 52, 65, 66, or 94. In some cases, the heavy chain variable region comprises an amino acid sequence having at least 98% sequence identity to the amino acid sequence as set forth in SEQ ID NOs: 42, 43, 44, 45, 46, 47, 71, 72, or 93 and the light chain variable region comprises an amino acid sequence having at least 98% sequence identity to the amino acid sequence as set forth in SEQ ID NOS: 48, 49, 50, 51, 52, 65, 66, or 94. In some cases, the heavy chain variable region comprises an amino acid sequence having at least 99% sequence identity to the amino acid sequence as set forth in SEQ ID NOs: 42, 43, 44, 45, 46, 47, 71, 72, or 93 and the light chain variable region comprises an amino acid sequence having at least 99% sequence identity to the amino acid sequence as set forth in SEQ ID NOS: 48, 49, 50, 51, 52, 65, 66, or 94. In some cases, the heavy chain variable region comprises an amino acid sequence having 100% sequence identity to the amino acid sequence as set forth in SEQ ID NOs: 42, 43, 44, 45, 46, 47, 71, 72, or 93 and the light chain variable region comprises an amino acid sequence having 100% sequence identity to the amino acid sequence as set forth in SEQ ID NOS: 48, 49, 50, 51, 52, 65, 66, or 94.

In some cases, the heavy chain variable region comprises an amino acid sequence as set forth in SEQ ID NO: 1, with from 0 to 3 amino acid modifications, and the light chain variable region comprises an amino acid sequence as set forth in SEQ ID NO: 2, with from 0 to 3 amino acid modifications. In some cases, the heavy chain variable region comprises an amino acid sequence as set forth in SEQ ID NO: 1, with from 0 to 2 amino acid modifications, and the light chain variable region comprises an amino acid sequence as set forth in SEQ ID NO: 2, with from 0 to 2 amino acid modifications. In some cases, the heavy chain variable region comprises an amino acid sequence as set forth in SEQ ID NO: 1, with from 0 to 1 amino acid modifications, and the light chain variable region comprises an amino acid sequence as set forth in SEQ ID NO: 2, with from 0 to 1 amino acid modifications. In some cases, the heavy chain variable region comprises an amino acid sequence as set forth in SEQ ID NO: 1, and the light chain variable region comprises an amino acid sequence as set forth in SEQ ID NO: 2.

In some cases, the heavy chain variable region comprises an amino acid sequence as set forth in SEQ ID NO: 9, with from 0 to 3 amino acid modifications, and the light chain variable region comprises an amino acid sequence as set forth in SEQ ID NO: 10, with from 0 to 3 amino acid modifications. In some cases, the heavy chain variable region comprises an amino acid sequence as set forth in SEQ ID NO: 9, with from 0 to 2 amino acid modifications, and the light chain variable region comprises an amino acid sequence as set forth in SEQ ID NO: 10, with from 0 to 2 amino acid modifications. In some cases, the heavy chain variable region comprises an amino acid sequence as set forth in SEQ ID NO: 9, with from 0 to 1 amino acid modifications, and the light chain variable region comprises an amino acid sequence as set forth in SEQ ID NO: 10, with from 0 to 1 amino acid modifications. In some cases, the heavy chain variable region comprises an amino acid sequence as set forth in SEQ ID NO: 9, and the light chain variable region comprises an amino acid sequence as set forth in SEQ ID NO: 10.

In some cases, the heavy chain variable region comprises an amino acid sequence as set forth in SEQ ID NO: 17, with from 0 to 3 amino acid modifications, and the light chain variable region comprises an amino acid sequence as set forth in SEQ ID NO: 18, with from 0 to 3 amino acid modifications. In some cases, the heavy chain variable region comprises an amino acid sequence as set forth in SEQ ID NO: 17, with from 0 to 2 amino acid modifications, and the light chain variable region comprises an amino acid sequence as set forth in SEQ ID NO: 18, with from 0 to 2 amino acid modifications. In some cases, the heavy chain variable region comprises an amino acid sequence as set forth in SEQ ID NO: 17, with from 0 to 1 amino acid modifications, and the light chain variable region comprises an amino acid sequence as set forth in SEQ ID NO: 18, with from 0 to 1 amino acid modifications. In some cases, the heavy chain variable region comprises an amino acid sequence as set forth in SEQ ID NO: 17, and the light chain variable region comprises an amino acid sequence as set forth in SEQ ID NO: 18.

In some cases, the heavy chain variable region comprises an amino acid sequence as set forth in SEQ ID NO: 25, with from 0 to 3 amino acid modifications, and the light chain variable region comprises an amino acid sequence as set forth in SEQ ID NO: 26, with from 0 to 3 amino acid modifications. In some cases, the heavy chain variable region comprises an amino acid sequence as set forth in SEQ ID NO: 25, with from 0 to 2 amino acid modifications, and the light chain variable region comprises an amino acid sequence as set forth in SEQ ID NO: 26, with from 0 to 2 amino acid modifications. In some cases, the heavy chain variable region comprises an amino acid sequence as set forth in SEQ ID NO: 25, with from 0 to 1 amino acid modifications, and the light chain variable region comprises an amino acid sequence as set forth in SEQ ID NO: 26, with from 0 to 1 amino acid modifications. In some cases, the heavy chain variable region comprises an amino acid sequence as set forth in SEQ ID NO: 25, and the light chain variable region comprises an amino acid sequence as set forth in SEQ ID NO: 26.

In some cases, the heavy chain variable region comprises an amino acid sequence as set forth in SEQ ID NO: 33, with from 0 to 3 amino acid modifications, and the light chain variable region comprises an amino acid sequence as set forth in SEQ ID NO: 34, with from 0 to 3 amino acid modifications. In some cases, the heavy chain variable region comprises an amino acid sequence as set forth in SEQ ID NO: 33, with from 0 to 2 amino acid modifications, and the light chain variable region comprises an amino acid sequence as set forth in SEQ ID NO: 34, with from 0 to 2 amino acid modifications. In some cases, the heavy chain variable region comprises an amino acid sequence as set forth in SEQ ID NO: 33, with from 0 to 1 amino acid modifications, and the light chain variable region comprises an amino acid sequence as set forth in SEQ ID NO: 34, with from 0 to 1 amino acid modifications. In some cases, the heavy chain variable region comprises an amino acid sequence as set forth in SEQ ID NO: 33, and the light chain variable region comprises an amino acid sequence as set forth in SEQ ID NO: 34.

In some cases, the heavy chain variable region comprises an amino acid sequence as set forth in SEQ ID NO: 71, with from 0 to 3 amino acid modifications, and the light chain variable region comprises an amino acid sequence as set forth in SEQ ID NO: 65, with from 0 to 3 amino acid modifications. In some cases, the heavy chain variable region comprises an amino acid sequence as set forth in SEQ ID NO: 71, with from 0 to 2 amino acid modifications, and the light chain variable region comprises an amino acid sequence as set forth in SEQ ID NO: 65, with from 0 to 2 amino acid modifications. In some cases, the heavy chain variable region comprises an amino acid sequence as set forth in SEQ ID NO: 71, with from 0 to 1 amino acid modifications, and the light chain variable region comprises an amino acid sequence as set forth in SEQ ID NO: 65, with from 0 to 1 amino acid modifications. In some cases, the heavy chain variable region comprises an amino acid sequence as set forth in SEQ ID NO: 71, and the light chain variable region comprises an amino acid sequence as set forth in SEQ ID NO: 65.

In some cases, the heavy chain variable region comprises an amino acid sequence as set forth in SEQ ID NO: 72, with from 0 to 3 amino acid modifications, and the light chain variable region comprises an amino acid sequence as set forth in SEQ ID NO: 65, with from 0 to 3 amino acid modifications. In some cases, the heavy chain variable region comprises an amino acid sequence as set forth in SEQ ID NO: 72, with from 0 to 2 amino acid modifications, and the light chain variable region comprises an amino acid sequence as set forth in SEQ ID NO: 65, with from 0 to 2 amino acid modifications. In some cases, the heavy chain variable region comprises an amino acid sequence as set forth in SEQ ID NO: 72, with from 0 to 1 amino acid modifications, and the light chain variable region comprises an amino acid sequence as set forth in SEQ ID NO: 65, with from 0 to 1 amino acid modifications. In some cases, the heavy chain variable region comprises an amino acid sequence as set forth in SEQ ID NO: 72, and the light chain variable region comprises an amino acid sequence as set forth in SEQ ID NO: 65.

In some cases, the heavy chain variable region comprises an amino acid sequence as set forth in any one of SEQ ID NOs: 1, 42, 43, 44, 45, 46, 47, 71, 72, or 93 with from 0 to 3 amino acid modifications, and the light chain variable region comprises an amino acid sequence as set forth in SEQ ID NOs: 2, 48, 49, 50, 51, 52, 65, 66, or 94, with from 0 to 3 amino acid modifications. In some cases, the heavy chain variable region comprises an amino acid sequence as set forth in any one of SEQ ID NOs: 1, 42, 43, 44, 45, 46, 47, 71, 72, or 93 with from 0 to 2 amino acid modifications, and the light chain variable region comprises an amino acid sequence as set forth in SEQ ID NOs: 2, 48, 49, 50, 51, 52, 65, 66, or 94, with from 0 to 2 amino acid modifications. In some cases, the heavy chain variable region comprises an amino acid sequence as set forth in any one of SEQ ID NOs: 1, 42, 43, 44, 45, 46, 47, 71, 72, or 93 with from 0 to 1 amino acid modifications, and the light chain variable region comprises an amino acid sequence as set forth in SEQ ID NOs: 2, 48, 49, 50, 51, 52, 65, 66, or 94, with from 0 to 1 amino acid modifications. In some cases, the heavy chain variable region comprises an amino acid sequence as set forth in any one of SEQ ID NOs: 1, 42, 43, 44, 45, 46, 47, 71, 72, or 93 and the light chain variable region comprises an amino acid sequence as set forth in SEQ ID NO: 2, 48, 49, 50, 51, 52, 65, 66, or 94.

In some cases, the heavy chain variable region comprises an amino acid sequence as set forth in any one of SEQ ID NOs: 42, 43, 44, 45, 46, 47, 71, 72, or 93 with from 0 to 3 amino acid modifications, and the light chain variable region comprises an amino acid sequence as set forth in SEQ ID NOs: 48, 49, 50, 51, 52, 65, 66, or 94 with from 0 to 3 amino acid modifications. In some cases, the heavy chain variable region comprises an amino acid sequence as set forth in any one of SEQ ID NOs: 42, 43, 44, 45, 46, 47, 71, 72, or 93 with from 0 to 2 amino acid modifications, and the light chain variable region comprises an amino acid sequence as set forth in SEQ ID NOs: 48, 49, 50, 51, 52, 65, 66, or 94 with from 0 to 2 amino acid modifications. In some cases, the heavy chain variable region comprises an amino acid sequence as set forth in any one of SEQ ID NOs: 42, 43, 44, 45, 46, 47, 71, 72, or 93 with from 0 to 1 amino acid modifications, and the light chain variable region comprises an amino acid sequence as set forth in SEQ ID NOs: 48, 49, 50, 51, 52, 65, 66, or 94 with from 0 to 1 amino acid modifications. In some cases, the heavy chain variable region comprises an amino acid sequence as set forth in any one of SEQ ID NOs: 42, 43, 44, 45, 46, 47, 71, 72, or 93 and the light chain variable region comprises an amino acid sequence as set forth in SEQ ID NOS: 48, 49, 50, 51, 52, 65, 66, or 94.

In some embodiments, the antibody provided herein is an IgG molecule. In some embodiments, the antibody provided herein is derived from an IgG molecule. In some embodiments, the IgG molecule is selected from IgG1, IgG2, IgG3, and IgG4. In some embodiments, the antibody provided herein is an IgG1 molecule. In some embodiments, the antibody provided herein is derived from an IgG1 molecule. In some embodiments, the antibody provided herein is an IgG4 molecule. In some embodiments, the antibody provided herein is derived from an IgG4 molecule. In some embodiments, the antibody provided herein is an IgM molecule. In some embodiments, the antibody provided herein is derived from an IgM molecule. In some embodiments, the antibody provided herein is an IgE molecule. In some embodiments, the antibody provided herein is derived from an IgE molecule In some embodiments, the antibody provided herein is an IgA molecule. In some embodiments, the antibody provided herein is derived from an IgA molecule. In some embodiments, the IgA molecule is an IgA1 or IgA2 molecule. In some embodiments, the antibody provided herein is an IgD molecule. In some embodiments, the antibody provided herein is derived from an IgD molecule.

In some cases, the antibody provided herein comprises a heavy chain constant region that comprises an amino acid sequence having at least 90% identity to any of SEQ ID NOs: 61, 63, 75, and 76. In some cases, the antibody provided herein comprises a heavy chain constant region that comprises an amino acid sequence having at least 95% identity to any of SEQ ID NOs: 61, 63, 75, and 76. In some cases, the antibody provided herein comprises a heavy chain constant region that comprises an amino acid sequence having at least 96% identity to any of SEQ ID NOs: 61, 63, 75, and 76. In some cases, the antibody provided herein comprises a heavy chain constant region that comprises an amino acid sequence having at least 97% identity to any of SEQ ID NOs: 61, 63, 75, and 76. In some cases, the antibody provided herein comprises a heavy chain constant region that comprises an amino acid sequence having at least 98% identity to any of SEQ ID NOs: 61, 63, 75, and 76. In some cases, the antibody provided herein comprises a heavy chain constant region that comprises an amino acid sequence having at least 99% identity to any of SEQ ID NOs: 61, 63, 75, and 76. In some cases, the antibody provided herein comprises a heavy chain constant region that comprises an amino acid sequence having about 95% identity to any of SEQ ID NOs: 61, 63, 75, and 76. In some cases, the antibody provided herein comprises a heavy chain constant region that comprises an amino acid sequence having about 98% identity to any of SEQ ID NOs: 61, 63, 75, and 76. In some cases, the antibody provided herein comprises a heavy chain constant region that comprises an amino acid sequence having about 99% identity to any of SEQ ID NOs: 61, 63, 75, and 76. In some cases, the antibody provided herein comprises a heavy chain constant region that comprises an amino acid sequence having 100% identity to any of SEQ ID NOs: 61, 63, 75, and 76.

In some cases, the antibody provided herein comprises a heavy chain variable region (HCVR) linked to a heavy chain constant region, wherein the heavy chain constant region comprises an amino acid sequence having at least 90%, 95%, 96%, 97%, 98%, 99%, or 100% identity to SEQ ID NO: 61. In some embodiments, the heavy chain constant region comprises an amino acid sequence having at least 90% sequence identity to the amino acid sequence as set forth in SEQ ID NO: 61. In some embodiments, the heavy chain constant region comprises an amino acid sequence having at least 95% sequence identity to the amino acid sequence as set forth in SEQ ID NO: 61. In some embodiments, the heavy chain constant region comprises an amino acid sequence having at least 97% sequence identity to the amino acid sequence as set forth in SEQ ID NO: 61. In some embodiments, the heavy chain constant region comprises an amino acid sequence having at least 98% sequence identity to the amino acid sequence as set forth in SEQ ID NO: 61. In some embodiments, the heavy chain constant region comprises an amino acid sequence having at least 99% sequence identity to the amino acid sequence as set forth in SEQ ID NO: 61. In some embodiments, the heavy chain constant region comprises an amino acid sequence having 100% sequence identity to the amino acid sequence as set forth in SEQ ID NO: 61. In some embodiments, the HCVR is linked to a heavy chain constant region comprising an amino acid sequence as set forth in SEQ ID NO: 61, with from 0 to 10 amino acid modifications, such as 1, 2, 3, 4, 5, 6, 7, 8, 9 or 10 amino acid modifications. In some embodiments, the heavy chain constant region comprises an amino acid sequence as set forth in SEQ ID NO: 61, with from 0 to 9 amino acid modifications. In some embodiments, the heavy chain constant region comprises an amino acid sequence as set forth in SEQ ID NO: 61, with from 0 to 8 amino acid modifications. In some embodiments, the heavy chain constant region comprises an amino acid sequence as set forth in SEQ ID NO: 61, with from 0 to 7 amino acid modifications. In some embodiments, the heavy chain constant region comprises an amino acid sequence as set forth in SEQ ID NO: 61, with from 0 to 6 amino acid modifications. In some embodiments, the heavy chain constant region comprises an amino acid sequence as set forth in SEQ ID NO: 61, with from 0 to 5 amino acid modifications. In some embodiments, the heavy chain constant region comprises an amino acid sequence as set forth in SEQ ID NO: 61, with from 0 to 4 amino acid modifications. In some embodiments, the heavy chain constant region comprises an amino acid sequence as set forth in SEQ ID NO: 61, with from 0 to 3 amino acid modifications. In some embodiments, the heavy chain constant region comprises an amino acid sequence as set forth in SEQ ID NO: 61, with from 0 to 2 amino acid modifications. In some embodiments, the heavy chain constant region comprises an amino acid sequence as set forth in SEQ ID NO: 61, with from 0 to 1 amino acid modifications. In some embodiments, the heavy chain constant region comprises an amino acid sequence as set forth in SEQ ID NO: 61.

In some cases, the antibody provided herein comprises a heavy chain variable region (HCVR) linked to a heavy chain constant region, wherein the heavy chain constant region comprises an amino acid sequence having at least 90%, 95%, 96%, 97%, 98%, 99%, or 100% identity to SEQ ID NO: 63. In some embodiments, the heavy chain constant region comprises an amino acid sequence having at least 90% sequence identity to the amino acid sequence as set forth in SEQ ID NO: 63. In some embodiments, the heavy chain constant region comprises an amino acid sequence having at least 95% sequence identity to the amino acid sequence as set forth in SEQ ID NO: 63. In some embodiments, the heavy chain constant region comprises an amino acid sequence having at least 97% sequence identity to the amino acid sequence as set forth in SEQ ID NO: 63. In some embodiments, the heavy chain constant region comprises an amino acid sequence having at least 98% sequence identity to the amino acid sequence as set forth in SEQ ID NO: 63. In some embodiments, the heavy chain constant region comprises an amino acid sequence having at least 99% sequence identity to the amino acid sequence as set forth in SEQ ID NO: 63. In some embodiments, the heavy chain constant region comprises an amino acid sequence having 100% sequence identity to the amino acid sequence as set forth in SEQ ID NO: 63. In some embodiments, the HCVR is linked to a heavy chain constant region comprising an amino acid sequence as set forth in SEQ ID NO: 63, with from 0 to 10 amino acid modifications, such as 1, 2, 3, 4, 5, 6, 7, 8, 9 or 10 amino acid modifications. In some embodiments, the heavy chain constant region comprises an amino acid sequence as set forth in SEQ ID NO: 63, with from 0 to 9 amino acid modifications. In some embodiments, the heavy chain constant region comprises an amino acid sequence as set forth in SEQ ID NO: 63, with from 0 to 8 amino acid modifications. In some embodiments, the heavy chain constant region comprises an amino acid sequence as set forth in SEQ ID NO: 63, with from 0 to 7 amino acid modifications. In some embodiments, the heavy chain constant region comprises an amino acid sequence as set forth in SEQ ID NO: 63, with from 0 to 6 amino acid modifications. In some embodiments, the heavy chain constant region comprises an amino acid sequence as set forth in SEQ ID NO: 63, with from 0 to 5 amino acid modifications. In some embodiments, the heavy chain constant region comprises an amino acid sequence as set forth in SEQ ID NO: 63, with from 0 to 4 amino acid modifications. In some embodiments, the heavy chain constant region comprises an amino acid sequence as set forth in SEQ ID NO: 63, with from 0 to 3 amino acid modifications. In some embodiments, the heavy chain constant region comprises an amino acid sequence as set forth in SEQ ID NO: 63, with from 0 to 2 amino acid modifications. In some embodiments, the heavy chain constant region comprises an amino acid sequence as set forth in SEQ ID NO: 63, with from 0 to 1 amino acid modifications. In some embodiments, the heavy chain constant region comprises an amino acid sequence as set forth in SEQ ID NO: 63.

In some cases, the antibody provided herein comprises a heavy chain variable region (HCVR) linked to a heavy chain constant region, wherein the heavy chain constant region comprises an amino acid sequence having at least 90% identity to SEQ ID NO: 75. In some cases, the antibody provided herein comprises a heavy chain constant region that comprises an amino acid sequence having at least 95% identity to SEQ ID NO: 75. In some cases, the antibody provided herein comprises a heavy chain constant region that comprises an amino acid sequence having at least 96% identity to SEQ ID NO: 75. In some cases, the antibody provided herein comprises a heavy chain constant region that comprises an amino acid sequence having at least 97% identity to SEQ ID NO: 75. In some cases, the antibody provided herein comprises a heavy chain constant region that comprises an amino acid sequence having at least 98% identity to SEQ ID NO: 75. In some cases, the antibody provided herein comprises a heavy chain constant region that comprises an amino acid sequence having at least 99% identity to SEQ ID NO: 75. In some cases, the antibody provided herein comprises a heavy chain constant region that comprises an amino acid sequence having 100% identity to SEQ ID NO: 75. In some embodiments, the heavy chain constant region comprises an amino acid sequence as set forth in SEQ ID NO: 75, with from 0 to 10 amino acid modifications, such as 1, 2, 3, 4, 5, 6, 7, 8, 9 or 10 amino acid modifications. In some embodiments, the heavy chain constant region comprises an amino acid sequence as set forth in SEQ ID NO: 75, with from 0 to 9 amino acid modifications. In some embodiments, the heavy chain constant region comprises an amino acid sequence as set forth in SEQ ID NO: 75, with from 0 to 8 amino acid modifications. In some embodiments, the heavy chain constant region comprises an amino acid sequence as set forth in SEQ ID NO: 75, with from 0 to 7 amino acid modifications. In some embodiments, the heavy chain constant region comprises an amino acid sequence as set forth in SEQ ID NO: 75, with from 0 to 6 amino acid modifications. In some embodiments, the heavy chain constant region comprises an amino acid sequence as set forth in SEQ ID NO: 75, with from 0 to 5 amino acid modifications. In some embodiments, the heavy chain constant region comprises an amino acid sequence as set forth in SEQ ID NO: 75, with from 0 to 4 amino acid modifications. In some embodiments, the heavy chain constant region comprises an amino acid sequence as set forth in SEQ ID NO: 75, with from 0 to 3 amino acid modifications. In some embodiments, the heavy chain constant region comprises an amino acid sequence as set forth in SEQ ID NO: 75, with from 0 to 2 amino acid modifications. In some embodiments, the heavy chain constant region comprises an amino acid sequence as set forth in SEQ ID NO: 75, with from 0 to 1 amino acid modifications. In some embodiments, the heavy chain constant region comprises an amino acid sequence as set forth in SEQ ID NO: 75.

In some cases, any of the antibodies provided herein comprises a heavy chain variable region (HCVR) linked to a heavy chain constant region, wherein the heavy chain constant region comprises an amino acid sequence having at least 90%, 95%, 96%, 97%, 98%, 99%, or 100% sequence identity to the amino acid sequence as set forth in SEQ ID NO: 76. In some embodiments, the heavy chain constant region comprises an amino acid sequence having at least 95% sequence identity to the amino acid sequence as set forth in SEQ ID NO: 76. In some embodiments, the heavy chain constant region comprises an amino acid sequence having at least 97% sequence identity to the amino acid sequence as set forth in SEQ ID NO: 76. In some embodiments, the heavy chain constant region comprises an amino acid sequence having at least 98% sequence identity to the amino acid sequence as set forth in SEQ ID NO: 76. In some embodiments, the heavy chain constant region comprises an amino acid sequence having at least 99% sequence identity to the amino acid sequence as set forth in SEQ ID NO: 76. In some embodiments, the heavy chain constant region comprises an amino acid sequence having 100% sequence identity to the amino acid sequence as set forth in SEQ ID NO: 76. In some embodiments, the HCVR is linked to a heavy chain constant region comprising an amino acid sequence as set forth in SEQ ID NO: 76, with from 0 to 10 amino acid modifications, such as 1, 2, 3, 4, 5, 6, 7, 8, 9 or 10 amino acid modifications. In some embodiments, the heavy chain constant region comprises an amino acid sequence as set forth in SEQ ID NO: 76, with from 0 to 9 amino acid modifications. In some embodiments, the heavy chain constant region comprises an amino acid sequence as set forth in SEQ ID NO: 76, with from 0 to 8 amino acid modifications. In some embodiments, the heavy chain constant region comprises an amino acid sequence as set forth in SEQ ID NO: 76, with from 0 to 7 amino acid modifications. In some embodiments, the heavy chain constant region comprises an amino acid sequence as set forth in SEQ ID NO: 76, with from 0 to 6 amino acid modifications. In some embodiments, the heavy chain constant region comprises an amino acid sequence as set forth in SEQ ID NO: 76, with from 0 to 5 amino acid modifications. In some embodiments, the heavy chain constant region comprises an amino acid sequence as set forth in SEQ ID NO: 76, with from 0 to 4 amino acid modifications. In some embodiments, the heavy chain constant region comprises an amino acid sequence as set forth in SEQ ID NO: 76, with from 0 to 3 amino acid modifications. In some embodiments, the heavy chain constant region comprises an amino acid sequence as set forth in SEQ ID NO: 76, with from 0 to 2 amino acid modifications. In some embodiments, the heavy chain constant region comprises an amino acid sequence as set forth in SEQ ID NO: 76, with from 0 to 1 amino acid modifications. In some embodiments, the HCVR is linked to a heavy chain constant region comprising an amino acid sequence as set forth in SEQ ID NO: 76. In some cases, the antibody provided herein comprises a heavy chain constant region that comprises the sequence set forth in SEQ ID NO: 76 but wherein Lys (K) at position 250 has been replaced by Gln (Q) or Glu (E).

In some cases, the antibody or antigen-binding fragment thereof provided herein comprises a light chain variable region (LCVR) is linked to a light chain constant region, wherein the light chain constant region comprises an amino acid sequence having at least 90%, 95%, 96%, 97%, 98%, 99%, or 100% sequence identity to the amino acid sequence as set forth in SEQ ID NO: 62. In some embodiments, light chain constant region comprises an amino acid sequence having at least 95% sequence identity to the amino acid sequence as set forth in SEQ ID NO: 62. In some embodiments, light chain constant region comprises an amino acid sequence having at least 97% sequence identity to the amino acid sequence as set forth in SEQ ID NO: 62. In some embodiments, light chain constant region comprises an amino acid sequence having at least 98% sequence identity to the amino acid sequence as set forth in SEQ ID NO: 62. In some embodiments, light chain constant region comprises an amino acid sequence having at least 99% sequence identity to the amino acid sequence as set forth in SEQ ID NO: 62. In some embodiments, light chain constant region comprises an amino acid sequence having 100% sequence identity to the amino acid sequence as set forth in SEQ ID NO: 62.

In some embodiments, any of the antibody or antigen-binding fragments disclosed herein comprise a light chain variable region linked to a light chain constant region, wherein the light chain constant region comprises an amino acid sequence as set forth in SEQ ID NO: 62, with from 0 to 10 amino acid modifications, such as 1, 2, 3, 4, 5, 6, 7, 8, 9 or 10 amino acid modifications. In some embodiments, light chain constant region comprises an amino acid sequence as set forth in SEQ ID NO: 62, with from 0 to 9 amino acid modifications. In some embodiments, light chain constant region comprises an amino acid sequence as set forth in SEQ ID NO: 62, with from 0 to 8 amino acid modifications. In some embodiments, light chain constant region comprises an amino acid sequence as set forth in SEQ ID NO: 62, with from 0 to 7 amino acid modifications. In some embodiments, light chain constant region comprises an amino acid sequence as set forth in SEQ ID NO: 62, with from 0 to 6 amino acid modifications. In some embodiments, light chain constant region comprises an amino acid sequence as set forth in SEQ ID NO: 62, with from 0 to 5 amino acid modifications. In some embodiments, light chain constant region comprises an amino acid sequence as set forth in SEQ ID NO: 62, with from 0 to 4 amino acid modifications. In some embodiments, light chain constant region comprises an amino acid sequence as set forth in SEQ ID NO: 62, with from 0 to 3 amino acid modifications. In some embodiments, light chain constant region comprises an amino acid sequence as set forth in SEQ ID NO: 62, with from 0 to 2 amino acid modifications. In some embodiments, light chain constant region comprises an amino acid sequence as set forth in SEQ ID NO: 62, with from 0 to 1 amino acid modifications. In some embodiments, light chain constant region comprises an amino acid sequence as set forth in SEQ ID NO: 62.

In some embodiments, any of the antibodies or antigen-binding fragments thereof disclosed herein comprise a light chain variable region linked to a light chain constant region, wherein the light chain constant region comprises an amino acid sequence having at least 90%, 95%, 96%, 97%, 98%, 99%, or 100% sequence identity to the amino acid sequence as set forth in SEQ ID NO: 64. In some embodiments, light chain constant region comprises an amino acid sequence having at least 95% sequence identity to the amino acid sequence as set forth in SEQ ID NO: 64. In some embodiments, light chain constant region comprises an amino acid sequence having at least 97% sequence identity to the amino acid sequence as set forth in SEQ ID NO: 64. In some embodiments, light chain constant region comprises an amino acid sequence having at least 98% sequence identity to the amino acid sequence as set forth in SEQ ID NO: 64. In some embodiments, light chain constant region comprises an amino acid sequence having at least 99% sequence identity to the amino acid sequence as set forth in SEQ ID NO: 64. In some embodiments, light chain constant region comprises an amino acid sequence having 100% sequence identity to the amino acid sequence as set forth in SEQ ID NO: 64.

In some embodiments, any of the antibodies or antigen-binding fragments thereof disclosed herein comprise a light chain variable region linked to a light chain constant region, wherein the light chain constant region comprises an amino acid sequence as set forth in SEQ ID NO: 64, with from 0 to 10 amino acid modifications, such as 1, 2, 3, 4, 5, 6, 7, 8, 9 or 10 amino acid modifications. In some embodiments, light chain constant region comprises an amino acid sequence as set forth in SEQ ID NO: 64, with from 0 to 9 amino acid modifications. In some embodiments, light chain constant region comprises an amino acid sequence as set forth in SEQ ID NO: 64, with from 0 to 8 amino acid modifications. In some embodiments, light chain constant region comprises an amino acid sequence as set forth in SEQ ID NO: 64, with from 0 to 7 amino acid modifications. In some embodiments, light chain constant region comprises an amino acid sequence as set forth in SEQ ID NO: 64, with from 0 to 6amino acid modifications. In some embodiments, light chain constant region comprises an amino acid sequence as set forth in SEQ ID NO: 64, with from 0 to 5 amino acid modifications. In some embodiments, light chain constant region comprises an amino acid sequence as set forth in SEQ ID NO: 64, with from 0 to 4 amino acid modifications. In some embodiments, light chain constant region comprises an amino acid sequence as set forth in SEQ ID NO: 64, with from 0 to 3 amino acid modifications. In some embodiments, light chain constant region comprises an amino acid sequence as set forth in SEQ ID NO: 64, with from 0 to 2 amino acid modifications. In some embodiments, light chain constant region comprises an amino acid sequence as set forth in SEQ ID NO: 64, with from 0 to 1 amino acid modifications. In some embodiments, light chain constant region comprises an amino acid sequence as set forth in SEQ ID NO: 64.

In some cases, any of the antibodies disclosed herein comprise a heavy chain constant region comprising an amino acid sequence having at least 90% sequence identity to the amino acid sequence as set forth in SEQ ID NO: 61, and the light chain constant region comprising an amino acid sequence having at least 90% sequence identity to the amino acid sequence as set forth in SEQ ID NO: 62. In some cases, the heavy chain constant region comprises an amino acid sequence having at least 95% sequence identity to the amino acid sequence as set forth in SEQ ID NO: 61, and the light chain constant region comprises an amino acid sequence having at least 95% sequence identity to the amino acid sequence as set forth in SEQ ID NO: 62. In some cases, the heavy chain constant region comprises an amino acid sequence having at least 96% sequence identity to the amino acid sequence as set forth in SEQ ID NO: 61, and the light chain constant region comprises an amino acid sequence having at least 96% sequence identity to the amino acid sequence as set forth in SEQ ID NO: 62. In some cases, the heavy chain constant region comprises an amino acid sequence having at least 97% sequence identity to the amino acid sequence as set forth in SEQ ID NO: 61, and the light chain constant region comprises an amino acid sequence having at least 97% sequence identity to the amino acid sequence as set forth in SEQ ID NO: 62. In some cases, the heavy chain constant region comprises an amino acid sequence having at least 98% sequence identity to the amino acid sequence as set forth in SEQ ID NO: 61, and the light chain constant region comprises an amino acid sequence having at least 98% sequence identity to the amino acid sequence as set forth in SEQ ID NO: 62. In some cases, the heavy chain constant region comprises an amino acid sequence having at least 99% sequence identity to the amino acid sequence as set forth in SEQ ID NO: 61, and the light chain constant region comprises an amino acid sequence having at least 99% sequence identity to the amino acid sequence as set forth in SEQ ID NO: 62. In some cases, the heavy chain constant region comprises an amino acid sequence having 100% sequence identity to the amino acid sequence as set forth in SEQ ID NO: 61, and the light chain constant region comprises an amino acid sequence having 100% sequence identity to the amino acid sequence as set forth in SEQ ID NO: 62.

In some cases, any of the antibodies disclosed herein comprise a heavy chain constant region comprising an amino acid sequence having at least 90% sequence identity to the amino acid sequence as set forth in SEQ ID NO: 75, and the light chain constant region comprising an amino acid sequence having at least 90% sequence identity to the amino acid sequence as set forth in SEQ ID NO: 62. In some cases, the heavy chain constant region comprises an amino acid sequence having at least 95% sequence identity to the amino acid sequence as set forth in SEQ ID NO: 75, and the light chain constant region comprises an amino acid sequence having at least 95% sequence identity to the amino acid sequence as set forth in SEQ ID NO: 62. In some cases, the heavy chain constant region comprises an amino acid sequence having at least 96% sequence identity to the amino acid sequence as set forth in SEQ ID NO: 75, and the light chain constant region comprises an amino acid sequence having at least 96% sequence identity to the amino acid sequence as set forth in SEQ ID NO: 62. In some cases, the heavy chain constant region comprises an amino acid sequence having at least 97% sequence identity to the amino acid sequence as set forth in SEQ ID NO: 75, and the light chain constant region comprises an amino acid sequence having at least 97% sequence identity to the amino acid sequence as set forth in SEQ ID NO: 62. In some cases, the heavy chain constant region comprises an amino acid sequence having at least 98% sequence identity to the amino acid sequence as set forth in SEQ ID NO: 75, and the light chain constant region comprises an amino acid sequence having at least 98% sequence identity to the amino acid sequence as set forth in SEQ ID NO: 62. In some cases, the heavy chain constant region comprises an amino acid sequence having at least 99% sequence identity to the amino acid sequence as set forth in SEQ ID NO: 75, and the light chain constant region comprises an amino acid sequence having at least 99% sequence identity to the amino acid sequence as set forth in SEQ ID NO: 62. In some cases, the heavy chain constant region comprises an amino acid sequence having 100% sequence identity to the amino acid sequence as set forth in SEQ ID NO: 75, and the light chain constant region comprises an amino acid sequence having 100% sequence identity to the amino acid sequence as set forth in SEQ ID NO: 62.

In some cases, any of the antibodies disclosed herein comprise a heavy chain constant region comprising an amino acid sequence having at least 90% sequence identity to the amino acid sequence as set forth in SEQ ID NO: 76, and the light chain constant region comprising an amino acid sequence having at least 90% sequence identity to the amino acid sequence as set forth in SEQ ID NO: 62. In some cases, the heavy chain constant region comprises an amino acid sequence having at least 95% sequence identity to the amino acid sequence as set forth in SEQ ID NO: 76, and the light chain constant region comprises an amino acid sequence having at least 95% sequence identity to the amino acid sequence as set forth in SEQ ID NO: 62. In some cases, the heavy chain constant region comprises an amino acid sequence having at least 96% sequence identity to the amino acid sequence as set forth in SEQ ID NO: 76, and the light chain constant region comprises an amino acid sequence having at least 96% sequence identity to the amino acid sequence as set forth in SEQ ID NO: 62. In some cases, the heavy chain constant region comprises an amino acid sequence having at least 97% sequence identity to the amino acid sequence as set forth in SEQ ID NO: 76, and the light chain constant region comprises an amino acid sequence having at least 97% sequence identity to the amino acid sequence as set forth in SEQ ID NO: 62. In some cases, the heavy chain constant region comprises an amino acid sequence having at least 98% sequence identity to the amino acid sequence as set forth in SEQ ID NO: 76, and the light chain constant region comprises an amino acid sequence having at least 98% sequence identity to the amino acid sequence as set forth in SEQ ID NO: 62. In some cases, the heavy chain constant region comprises an amino acid sequence having at least 99% sequence identity to the amino acid sequence as set forth in SEQ ID NO: 76, and the light chain constant region comprises an amino acid sequence having at least 99% sequence identity to the amino acid sequence as set forth in SEQ ID NO: 62. In some cases, the heavy chain constant region comprises an amino acid sequence having 100% sequence identity to the amino acid sequence as set forth in SEQ ID NO: 76, and the light chain constant region comprises an amino acid sequence having 100% sequence identity to the amino acid sequence as set forth in SEQ ID NO: 62.

In some cases, any of the antibodies disclosed herein comprise a heavy chain constant region comprising an amino acid sequence as set forth in any one of SEQ ID NOs: 61, 63, 75, and 76, with from 0 to 3 amino acid modifications, and the light chain constant region comprising an amino acid sequence as set forth in SEQ ID NOs: 62 and 64, with from 0 to 3 amino acid modifications. In some cases, the heavy chain constant region comprises an amino acid sequence as set forth in any one of SEQ ID NOs: 61, 63, 75, and 76, with from 0 to 2 amino acid modifications, and the light chain constant region comprises an amino acid sequence as set forth in SEQ ID NOs: 62 and 64, with from 0 to 2 amino acid modifications. In some cases, the heavy chain constant region comprises an amino acid sequence as set forth in any one of SEQ ID NOs: 61, 63, 75, and 76, with from 0 to 1 amino acid modifications, and the light chain constant region comprises an amino acid sequence as set forth in SEQ ID NOs: 62 and 64, with from 0 to 1 amino acid modifications. In some cases, the heavy chain constant region comprises an amino acid sequence as set forth in any one of SEQ ID NOs: 61, 63, 75, and 76, and the light chain constant region comprises an amino acid sequence as set forth in SEQ ID NOs: 62 and 64.

In some cases, any of the antibodies disclosed herein comprise a heavy chain constant region comprising an amino acid sequence as set forth in any one of SEQ ID NOs: 61, 75, and 76, with from 0 to 3 amino acid modifications, and the light chain constant region comprising an amino acid sequence as set forth in SEQ ID NO: 62, with from 0 to 3 amino acid modifications. In some cases, the heavy chain constant region comprises an amino acid sequence as set forth in any one of SEQ ID NOs: 61, 75, and 76, with from 0 to 2 amino acid modifications, and the light chain constant region comprises an amino acid sequence as set forth in SEQ ID NO: 62, with from 0 to 2 amino acid modifications. In some cases, the heavy chain constant region comprises an amino acid sequence as set forth in any one of SEQ ID NOs: 61, 75, and 76, with from 0 to 1 amino acid modifications, and the light chain constant region comprises an amino acid sequence as set forth in SEQ ID NO: 62, with from 0 to 1 amino acid modifications. In some cases, the heavy chain constant region comprises an amino acid sequence as set forth in any one of SEQ ID NOs: 61, 75, and 76, and the light chain constant region comprises an amino acid sequence as set forth in SEQ ID NO: 62.

In some cases, any of the antibodies disclosed herein comprise heavy chain constant region comprising an amino acid sequence as set forth in any one of SEQ ID NO: 61, with from 0 to 10 amino acid modifications, such as 1, 2, 3, 4, 5, 6, 7, 8, 9 or 10 amino acid modifications, and the light chain constant region comprising an amino acid sequence as set forth in SEQ ID NO: 62, with from 0 to 10 amino acid modifications, such as 1, 2, 3, 4, 5, 6, 7, 8, 9 or 10 amino acid modifications. In some cases, the heavy chain constant region comprises an amino acid sequence as set forth in any one of SEQ ID NO: 61, with from 0 to 7 amino acid modifications, and the light chain constant region comprises an amino acid sequence as set forth in SEQ ID NO: 62, with from 0 to 7 amino acid modifications. In some cases, the heavy chain constant region comprises an amino acid sequence as set forth in any one of SEQ ID NO: 61, with from 0 to 5 amino acid modifications, and the light chain constant region comprises an amino acid sequence as set forth in SEQ ID NO: 62, with from 0 to 5 amino acid modifications. In some cases, the heavy chain constant region comprises an amino acid sequence as set forth in any one of SEQ ID NO: 61, with from 0 to 4 amino acid modifications, and the light chain constant region comprises an amino acid sequence as set forth in SEQ ID NO: 62, with from 0 to 4 amino acid modifications. In some cases, the heavy chain constant region comprises an amino acid sequence as set forth in any one of SEQ ID NO: 61, with from 0 to 3 amino acid modifications, and the light chain constant region comprises an amino acid sequence as set forth in SEQ ID NO: 62, with from 0 to 3 amino acid modifications. In some cases, the heavy chain constant region comprises an amino acid sequence as set forth in any one of SEQ ID NO: 61, with from 0 to 2 amino acid modifications, and the light chain constant region comprises an amino acid sequence as set forth in SEQ ID NO: 62, with from 0 to 2 amino acid modifications. In some cases, the heavy chain constant region comprises an amino acid sequence as set forth in any one of SEQ ID NO: 61, with from 0 to 1 amino acid modifications, and the light chain constant region comprises an amino acid sequence as set forth in SEQ ID NO: 62, with from 0 to 1 amino acid modifications. In some cases, the heavy chain constant region comprises an amino acid sequence as set forth in any one of SEQ ID NO: 61, and the light chain constant region comprises an amino acid sequence as set forth in SEQ ID NO: 62.

In some cases, any of the antibodies disclosed herein comprise a heavy chain constant region comprising an amino acid sequence as set forth in any one of SEQ ID NO: 75, with from 0 to 10 amino acid modifications, such as 1, 2, 3, 4, 5, 6, 7, 8, 9 or 10 amino acid modifications, and the light chain constant region comprising an amino acid sequence as set forth in SEQ ID NO: 62, with from 0 to 10 amino acid modifications, such as 1, 2, 3, 4, 5, 6, 7, 8, 9 or 10 amino acid modifications. In some cases, the heavy chain constant region comprises an amino acid sequence as set forth in any one of SEQ ID NO: 75, with from 0 to 7 amino acid modifications, and the light chain constant region comprises an amino acid sequence as set forth in SEQ ID NO: 62, with from 0 to 7 amino acid modifications. In some cases, the heavy chain constant region comprises an amino acid sequence as set forth in any one of SEQ ID NO: 75, with from 0 to 5 amino acid modifications, and the light chain constant region comprises an amino acid sequence as set forth in SEQ ID NO: 62, with from 0 to 5 amino acid modifications. In some cases, the heavy chain constant region comprises an amino acid sequence as set forth in any one of SEQ ID NO: 75, with from 0 to 4 amino acid modifications, and the light chain constant region comprises an amino acid sequence as set forth in SEQ ID NO: 62, with from 0 to 4 amino acid modifications. In some cases, the heavy chain constant region comprises an amino acid sequence as set forth in any one of SEQ ID NO: 75, with from 0 to 3 amino acid modifications, and the light chain constant region comprises an amino acid sequence as set forth in SEQ ID NO: 62, with from 0 to 3 amino acid modifications. In some cases, the heavy chain constant region comprises an amino acid sequence as set forth in any one of SEQ ID NO: 75, with from 0 to 2 amino acid modifications, and the light chain constant region comprises an amino acid sequence as set forth in SEQ ID NO: 62, with from 0 to 2 amino acid modifications. In some cases, the heavy chain constant region comprises an amino acid sequence as set forth in any one of SEQ ID NO: 75, with from 0 to 1 amino acid modifications, and the light chain constant region comprises an amino acid sequence as set forth in SEQ ID NO: 62, with from 0 to 1 amino acid modifications. In some cases, the heavy chain constant region comprises an amino acid sequence as set forth in any one of SEQ ID NO: 75, and the light chain constant region comprises an amino acid sequence as set forth in SEQ ID NO: 62.

In some cases, any of the antibodies disclosed herein comprise a heavy chain constant region comprising an amino acid sequence as set forth in any one of SEQ ID NO: 76, with from 0 to 10 amino acid modifications, such as 1, 2, 3, 4, 5, 6, 7, 8, 9 or 10 amino acid modifications, and the light chain constant region comprising an amino acid sequence as set forth in SEQ ID NO: 62, with from 0 to 10 amino acid modifications, such as 1, 2, 3, 4, 5, 6, 7, 8, 9 or 10 amino acid modifications. In some cases, the heavy chain constant region comprises an amino acid sequence as set forth in any one of SEQ ID NO: 76, with from 0 to 7 amino acid modifications, and the light chain constant region comprises an amino acid sequence as set forth in SEQ ID NO: 62, with from 0 to 7 amino acid modifications. In some cases, the heavy chain constant region comprises an amino acid sequence as set forth in any one of SEQ ID NO: 76, with from 0 to 5 amino acid modifications, and the light chain constant region comprises an amino acid sequence as set forth in SEQ ID NO: 62, with from 0 to 5 amino acid modifications. In some cases, the heavy chain constant region comprises an amino acid sequence as set forth in any one of SEQ ID NO: 76, with from 0 to 4 amino acid modifications, and the light chain constant region comprises an amino acid sequence as set forth in SEQ ID NO: 62, with from 0 to 4 amino acid modifications. In some cases, the heavy chain constant region comprises an amino acid sequence as set forth in any one of SEQ ID NO: 76, with from 0 to 3 amino acid modifications, and the light chain constant region comprises an amino acid sequence as set forth in SEQ ID NO: 62, with from 0 to 3 amino acid modifications. In some cases, the heavy chain constant region comprises an amino acid sequence as set forth in any one of SEQ ID NO: 76, with from 0 to 2 amino acid modifications, and the light chain constant region comprises an amino acid sequence as set forth in SEQ ID NO: 62, with from 0 to 2 amino acid modifications. In some cases, the heavy chain constant region comprises an amino acid sequence as set forth in any one of SEQ ID NO: 76, with from 0 to 1 amino acid modifications, and the light chain constant region comprises an amino acid sequence as set forth in SEQ ID NO: 62, with from 0 to 1 amino acid modifications. In some cases, the heavy chain constant region comprises an amino acid sequence as set forth in any one of SEQ ID NO: 76, and the light chain constant region comprises an amino acid sequence as set forth in SEQ ID NO: 62.

Effector Functions

In some embodiments, the antibody described herein does not inhibit binding of CD200 to CD200R. In some embodiments, the antibody described herein does not compete with the binding of CD200 to CD200R.

In some embodiments, the antibody described herein does not induce significant cytokine release when the antibody binds to CD200R on the surface of an immune cell. In some embodiments, the antibody described herein does not induce significant increase in chemokine production when the antibody binds to CD200R on the surface of an immune cell. In some embodiments, the antibody described herein does not induce significant increase in any other activation markers of an immune cell that is in contact with the antibody.

In some embodiments, the antibody described herein comprises a domain that binds to an Fc receptor. In some embodiments, the Fc receptor is expressed on the surface of an immune cell. In some embodiments, the immune cell is an antigen presenting cell. In some embodiments, the antigen presenting cell is dendritic cell. In some embodiments, the antigen presenting cell is macrophage. In some embodiments, the antigen presenting cell is monocyte. In some embodiments, the antigen presenting cell is neutrophil.

In some embodiments, the binding of the antibody described herein to the Fc receptor expressed on the surface of the immune cell and the binding of the antibody to CD200R on the surface of a second immune cell results in the cell surface of the immune cell and the cell surface of the second immune cell to be within 250 Å. In some embodiments, the binding of the antibody described herein to the Fc receptor expressed on the surface of the immune cell and the binding of the antibody to CD200R on the surface of a second immune cell results in the cell surface of the immune cell and the cell surface of the second immune cell to be within 200 Å. In some embodiments, the binding of the antibody described herein to the Fc receptor expressed on the surface of the immune cell and the binding of the antibody to CD200R on the surface of a second immune cell results in the cell surface of the immune cell and the cell surface of the second immune cell to be within 150 Å. In some embodiments, the binding of the antibody described herein to the Fc receptor expressed on the surface of the immune cell and the binding of the antibody to CD200R on the surface of a second immune cell results in the cell surface of the immune cell and the cell surface of the second immune cell to be within 100 Å. In some embodiments, the binding of the antibody described herein to the Fc receptor expressed on the surface of the immune cell and the binding of the antibody to CD200R on the surface of a second immune cell results in the cell surface of the immune cell and the cell surface of the second immune cell to be within 50 Å.

In some embodiments, the Fc receptor expressed on the surface of the immune cell is FcγRIIB.

In some embodiments, the antibody disclosed herein reduces activation of an immune cell relative to a comparable immune cell not bound by the antibody. In some embodiments, the reduction in activation of an immune cell relative to a comparable immune cell not bound by the antibody is measured by an assay described in Example 5, 16, or 17. In some embodiments, the antibody disclosed herein decreases proliferation of an immune cell relative to a comparable immune cell not bound by the antibody. In some embodiments, the decrease in proliferation of an immune cell relative to a comparable immune cell not bound by the antibody is measured by an assay described in Example 5, 16, or 17. In some embodiments, the decrease in proliferation of an immune cell relative to a comparable immune cell not bound by the antibody is measured in vitro. In some embodiment, the decrease in proliferation of an immune cell relative to a comparable immune cell not bound by the antibody is measured in vivo. In some cases, the decrease in proliferation of an immune cell or immune cell population relative to a comparable immune cell or immune cell population not bound by the antibody is at least about 10%. In some cases, the decrease in proliferation of an immune cell or immune cell population relative to a comparable immune or immune cell population cell not bound by the antibody is at least about 15%. In some cases, the decrease in proliferation of an immune cell or immune cell population relative to a comparable immune cell or immune cell population not bound by the antibody is at least about 20%. In some cases, the decrease in proliferation of an immune cell or immune cell population relative to a comparable immune cell or immune cell population not bound by the antibody is at least about 25%. In some cases, the decrease in proliferation of an immune cell or immune cell population relative to a comparable immune cell or immune cell population not bound by the antibody is at least about 30%. In some cases, the decrease in proliferation of an immune cell or immune cell population relative to a comparable immune cell or immune cell population not bound by the antibody is at least about 35%. In some cases, the decrease in proliferation of an immune cell or immune cell population relative to a comparable immune cell or immune cell population not bound by the antibody is at least about 40%. In some cases, the decrease in proliferation of an immune cell or immune cell population relative to a comparable immune cell or immune cell population not bound by the antibody is at least about 45%. In some cases, the decrease in proliferation of an immune cell or immune cell population relative to a comparable immune cell or immune cell population not bound by the antibody is at least about 50%. In some cases, the decrease in proliferation of an immune cell or immune cell population relative to a comparable immune cell or immune cell population not bound by the antibody is at least about 10% to 50%. In some cases, the decrease in proliferation of an immune cell or immune cell population relative to a comparable immune cell or immune cell population not bound by the antibody is at least about 10% to 45%. In some cases, the decrease in proliferation of an immune cell or immune cell population relative to a comparable immune cell or immune cell population not bound by the antibody is at least about 10% to 40%. In some cases, the decrease in proliferation of an immune cell or immune cell population relative to a comparable immune cell or immune cell population not bound by the antibody is at least about 10% to 35%. In some cases, the decrease in proliferation of an immune cell or immune cell population relative to a comparable immune cell or immune cell population not bound by the antibody is at least about 10% to 30%. In some cases, the decrease in proliferation of an immune cell or immune cell population relative to a comparable immune cell or immune cell population not bound by the antibody is at least about 10% to 25%. In some cases, the decrease in proliferation of an immune cell or immune cell population relative to a comparable immune cell or immune cell population not bound by the antibody is at least about 10% to 20%. In some cases, the decrease in proliferation of an immune cell or immune cell population relative to a comparable immune cell or immune cell population not bound by the antibody is at least about 10% to 15%. In some cases, the decrease in proliferation of an immune cell or immune cell population relative to a comparable immune cell or immune cell population not bound by the antibody is at least about 15% to 50%. In some cases, the decrease in proliferation of an immune cell or immune cell population relative to a comparable immune cell or immune cell population not bound by the antibody is at least about 15% to 45%. In some cases, the decrease in proliferation of an immune cell or immune cell population relative to a comparable immune cell or immune cell population not bound by the antibody is at least about 15% to 40%. In some cases, the decrease in proliferation of an immune cell or immune cell population relative to a comparable immune cell or immune cell population not bound by the antibody is at least about 15% to 35%. In some cases, the decrease in proliferation of an immune cell or immune cell population relative to a comparable immune cell or immune cell population not bound by the antibody is at least about 15% to 30%. In some cases, the decrease in proliferation of an immune cell or immune cell population relative to a comparable immune cell or immune cell population not bound by the antibody is at least about 15% to 25%. In some cases, the decrease in proliferation of an immune cell or immune cell population relative to a comparable immune cell or immune cell population not bound by the antibody is at least about 15% to 20%. In some cases, the decrease in proliferation of an immune cell or immune cell population relative to a comparable immune cell or immune cell population not bound by the antibody is at least about 20% to 50%. In some cases, the decrease in proliferation of an immune cell or immune cell population relative to a comparable immune cell or immune cell population not bound by the antibody is at least about 20% to 45%. In some cases, the decrease in proliferation of an immune cell or immune cell population relative to a comparable immune cell or immune cell population not bound by the antibody is at least about 20% to 40%. In some cases, the decrease in proliferation of an immune cell or immune cell population relative to a comparable immune cell or immune cell population not bound by the antibody is at least about 20% to 35%. In some cases, the decrease in proliferation of an immune cell or immune cell population relative to a comparable immune cell or immune cell population not bound by the antibody is at least about 20% to 30%. In some cases, the decrease in proliferation of an immune cell or immune cell population relative to a comparable immune cell or immune cell population not bound by the antibody is at least about 20% to 25%. In some cases, the decrease in proliferation of an immune cell or immune cell population relative to a comparable immune cell or immune cell population not bound by the antibody is at least about 25% to 50%. In some cases, the decrease in proliferation of an immune cell or immune cell population relative to a comparable immune cell or immune cell population not bound by the antibody is at least about 25% to 45%. In some cases, the decrease in proliferation of an immune cell or immune cell population relative to a comparable immune cell or immune cell population not bound by the antibody is at least about 25% to 40%. In some cases, the decrease in proliferation of an immune cell or immune cell population relative to a comparable immune cell or immune cell population not bound by the antibody is at least about 25% to 35%. In some cases, the decrease in proliferation of an immune cell or immune cell population relative to a comparable immune cell or immune cell population not bound by the antibody is at least about 25% to 30%.

In some embodiments, the binding of the antibody disclosed herein to CD200R expressed on the surface of an immune cell decreases NFκB signaling of the immune cell relative to a comparable immune cell not bound by the antibody. In some embodiments, the decrease in NFκB signaling of the immune cell relative to a comparable immune cell not bound by the antibody is measured by an assay described in Example 5. In some embodiments, the decrease in NFκB signaling of the immune cell relative to a comparable immune cell not bound by the antibody is at least about 10%. In some embodiments, the decrease in NFκB signaling of the immune cell relative to a comparable immune cell not bound by the antibody is at least about 15%. In some embodiments, the decrease in NFκB signaling of the immune cell relative to a comparable immune cell not bound by the antibody is at least about 20%. In some embodiments, the decrease in NFκB signaling of the immune cell relative to a comparable immune cell not bound by the antibody is at least about 25%. In some embodiments, the decrease in NFκB signaling of the immune cell relative to a comparable immune cell not bound by the antibody is at least about 30%. In some embodiments, the decrease in NFκB signaling of the immune cell relative to a comparable immune cell not bound by the antibody is at least about 35%. In some embodiments, the decrease in NFκB signaling of the immune cell relative to a comparable immune cell not bound by the antibody is at least about 40%. In some embodiments, the decrease in NFκB signaling of the immune cell relative to a comparable immune cell not bound by the antibody is at least about 10% to 40%. In some embodiments, the decrease in NFκB signaling of the immune cell relative to a comparable immune cell not bound by the antibody is at least about 10% to 35%. In some embodiments, the decrease in NFκB signaling of the immune cell relative to a comparable immune cell not bound by the antibody is at least about 10% to 30%. In some embodiments, the decrease in NFκB signaling of the immune cell relative to a comparable immune cell not bound by the antibody is at least about 10% to 25%. In some embodiments, the decrease in NFκB signaling of the immune cell relative to a comparable immune cell not bound by the antibody is at least about 10% to 20%. In some embodiments, the decrease in NFκB signaling of the immune cell relative to a comparable immune cell not bound by the antibody is at least about 10% to 15%. In some embodiments, the decrease in NFκB signaling of the immune cell relative to a comparable immune cell not bound by the antibody is at least about 15% to 40%. In some embodiments, the decrease in NFκB signaling of the immune cell relative to a comparable immune cell not bound by the antibody is at least about 15% to 35%. In some embodiments, the decrease in NFκB signaling of the immune cell relative to a comparable immune cell not bound by the antibody is at least about 15% to 30%. In some embodiments, the decrease in NFκB signaling of the immune cell relative to a comparable immune cell not bound by the antibody is at least about 15% to 25%. In some embodiments, the decrease in NFκB signaling of the immune cell relative to a comparable immune cell not bound by the antibody is at least about 15% to 20%. In some embodiments, the decrease in NFκB signaling of the immune cell relative to a comparable immune cell not bound by the antibody is at least about 20% to 40%. In some embodiments, the decrease in NFκB signaling of the immune cell relative to a comparable immune cell not bound by the antibody is at least about 20% to 35%. In some embodiments, the decrease in NFκB signaling of the immune cell relative to a comparable immune cell not bound by the antibody is at least about 20% to 30%. In some embodiments, the decrease in NFκB signaling of the immune cell relative to a comparable immune cell not bound by the antibody is at least about 20% to 25%. In some embodiments, the decrease in NFκB signaling of the immune cell relative to a comparable immune cell not bound by the antibody is at least about 25% to 40%. In some embodiments, the decrease in NFκB signaling of the immune cell relative to a comparable immune cell not bound by the antibody is at least about 25% to 35%. In some embodiments, the decrease in NFκB signaling of the immune cell relative to a comparable immune cell not bound by the antibody is at least about 25% to 30%. In some embodiments, the decrease in NFκB signaling of the immune cell relative to a comparable immune cell not bound by the antibody is at least about 30% to 40%. In some embodiments, the decrease in NFκB signaling of the immune cell relative to a comparable immune cell not bound by the antibody is at least about 30% to 35%. In some embodiments, the decrease in NFκB signaling of the immune cell relative to a comparable immune cell not bound by the antibody is at least about 35% to 40%.

In some embodiments, an average maximal percentage inhibition of NFκB signaling of the immune cell induced by the antibody described herein at least 20% greater than a control antibody that comprises a control heavy chain comprising CDRH1, CDRH2, and CDRH3, which comprise amino acid sequence as set forth in SEQ ID NOs: 55-57, respectively and a control light chain comprising CDRL1, CDRL2, and CDRL3, which comprise amino acid sequence as set forth in SEQ ID NOs: 58-60, respectively. In some embodiments, an average maximal percentage inhibition of NFκB signaling of the immune cell induced by the antibody described herein at least 25% greater than a control antibody that comprises a control heavy chain comprising CDRH1, CDRH2, and CDRH3, which comprise amino acid sequence as set forth in SEQ ID NOs: 55-57, respectively and a control light chain comprising CDRL1, CDRL2, and CDRL3, which comprise amino acid sequence as set forth in SEQ ID NOs: 58-60, respectively. In some embodiments, an average maximal percentage inhibition of NFκB signaling of the immune cell induced by the antibody described herein at least 30% greater than a control antibody that comprises a control heavy chain comprising CDRH1, CDRH2, and CDRH3, which comprise amino acid sequence as set forth in SEQ ID NOs: 55-57, respectively and a control light chain comprising CDRL1, CDRL2, and CDRL3, which comprise amino acid sequence as set forth in SEQ ID NOs: 58-60, respectively. In some embodiments, an average maximal percentage inhibition of NFκB signaling of the immune cell induced by the antibody described herein at least 35% greater than a control antibody that comprises a control heavy chain comprising CDRH1, CDRH2, and CDRH3, which comprise amino acid sequence as set forth in SEQ ID NOs: 55-57, respectively and a control light chain comprising CDRL1, CDRL2, and CDRL3, which comprise amino acid sequence as set forth in SEQ ID NOs: 58-60, respectively. In some embodiments, an average maximal percentage inhibition of NFκB signaling of the immune cell induced by the antibody described herein at least 40% greater than a control antibody that comprises a control heavy chain comprising CDRH1, CDRH2, and CDRH3, which comprise amino acid sequence as set forth in SEQ ID NOs: 55-57, respectively and a control light chain comprising CDRL1, CDRL2, and CDRL3, which comprise amino acid sequence as set forth in SEQ ID NOs: 58-60, respectively. In some embodiments, an average maximal percentage inhibition of NFκB signaling of the immune cell induced by the antibody described herein at least 45% greater than a control antibody that comprises a control heavy chain comprising CDRH1, CDRH2, and CDRH3, which comprise amino acid sequence as set forth in SEQ ID NOs: 55-57, respectively and a control light chain comprising CDRL1, CDRL2, and CDRL3, which comprise amino acid sequence as set forth in SEQ ID NOs: 58-60, respectively. In some embodiments, an average maximal percentage inhibition of NFκB signaling of the immune cell induced by the antibody described herein at least 50% greater than a control antibody that comprises a control heavy chain comprising CDRH1, CDRH2, and CDRH3, which comprise amino acid sequence as set forth in SEQ ID NOs: 55-57, respectively and a control light chain comprising CDRL1, CDRL2, and CDRL3, which comprise amino acid sequence as set forth in SEQ ID NOs: 58-60, respectively. In some embodiments, an average maximal percentage inhibition of NFκB signaling of the immune cell induced by the antibody described herein at least 55% greater than a control antibody that comprises a control heavy chain comprising CDRH1, CDRH2, and CDRH3, which comprise amino acid sequence as set forth in SEQ ID NOs: 55-57, respectively and a control light chain comprising CDRL1, CDRL2, and CDRL3, which comprise amino acid sequence as set forth in SEQ ID NOs: 58-60, respectively. In some embodiments, an average maximal percentage inhibition of NFκB signaling of the immune cell induced by the antibody described herein at least 60% greater than a control antibody that comprises a control heavy chain comprising CDRH1, CDRH2, and CDRH3, which comprise amino acid sequence as set forth in SEQ ID NOs: 55-57, respectively and a control light chain comprising CDRL1, CDRL2, and CDRL3, which comprise amino acid sequence as set forth in SEQ ID NOs: 58-60, respectively.

In some embodiments, an average maximal percentage inhibition of NFκB signaling of the immune cell induced by the antibody described herein at least 20% greater than a control antibody that comprises a control heavy chain variable region comprising amino acid sequence as set forth in SEQ ID NO: 53, and a control light chain variable region comprising amino acid sequence as set forth in SEQ ID NO: 54. In some embodiments, an average maximal percentage inhibition of NFκB signaling of the immune cell induced by the antibody described herein at least 25% greater than a control antibody that comprises a control heavy chain variable region comprising amino acid sequence as set forth in SEQ ID NO: 53, and a control light chain variable region comprising amino acid sequence as set forth in SEQ ID NO: 54. In some embodiments, an average maximal percentage inhibition of NFκB signaling of the immune cell induced by the antibody described herein at least 30% greater than a control antibody that comprises a control heavy chain variable region comprising amino acid sequence as set forth in SEQ ID NO: 53, and a control light chain variable region comprising amino acid sequence as set forth in SEQ ID NO: 54. In some embodiments, an average maximal percentage inhibition of NFκB signaling of the immune cell induced by the antibody described herein at least 35% greater than a control antibody that comprises a control heavy chain variable region comprising amino acid sequence as set forth in SEQ ID NO: 53, and a control light chain variable region comprising amino acid sequence as set forth in SEQ ID NO: 54. In some embodiments, an average maximal percentage inhibition of NFκB signaling of the immune cell induced by the antibody described herein at least 40% greater than a control antibody that comprises a control heavy chain variable region comprising amino acid sequence as set forth in SEQ ID NO: 53, and a control light chain variable region comprising amino acid sequence as set forth in SEQ ID NO: 54. In some embodiments, an average maximal percentage inhibition of NFκB signaling of the immune cell induced by the antibody described herein at least 45% greater than a control antibody that comprises a control heavy chain variable region comprising amino acid sequence as set forth in SEQ ID NO: 53, and a control light chain variable region comprising amino acid sequence as set forth in SEQ ID NO: 54. In some embodiments, an average maximal percentage inhibition of NFκB signaling of the immune cell induced by the antibody described herein at least 50% greater than a control antibody that comprises a control heavy chain variable region comprising amino acid sequence as set forth in SEQ ID NO: 53, and a control light chain variable region comprising amino acid sequence as set forth in SEQ ID NO: 54. In some embodiments, an average maximal percentage inhibition of NFκB signaling of the immune cell induced by the antibody described herein at least 55% greater than a control antibody that comprises a control heavy chain variable region comprising amino acid sequence as set forth in SEQ ID NO: 53, and a control light chain variable region comprising amino acid sequence as set forth in SEQ ID NO: 54. In some embodiments, an average maximal percentage inhibition of NFκB signaling of the immune cell induced by the antibody described herein at least 60% greater than a control antibody that comprises a control heavy chain variable region comprising amino acid sequence as set forth in SEQ ID NO: 53, and a control light chain variable region comprising amino acid sequence as set forth in SEQ ID NO: 54.

Basophils can express the highest levels of CD200R among human leukocytes and can also be strongly positive for FcγRIIb. In some embodiments, the antibody disclosed herein inhibits activation of basophils, for instance, inhibits activation of FcεRI-induced activation of basophils (e.g., activation induced by binding of IgE to the basophils). Without wishing to be bound by a certain theory, inhibition of basophils by a CD200R antibody disclosed herein may work through CD200R agonism and optionally through FcγRIIb. Inhibition of basophils by a CD200R antibody disclosed herein may provide therapeutic benefit in patients with a range of inflammatory conditions.

In some embodiments, the antibody disclosed herein inhibits activation of basophils by at least 10%, 15%, 20%, 25%, 30%, 35%, 40%, 45%, 50%, 55%, 60%, 65%, 70%, 80%, or 90%.

In some cases, the antibody disclosed herein inhibits activation of basophils by at least 50%. In some cases, the antibody disclosed herein inhibits activation of basophils by about 10% to about 90%, such as, about 10% to about 80%, about 10% to about 60%, about 10% to about 50%, about 20% to about 70%, about 20% to about 50%, about 20% to about 40%, about 30% to about 60%, about 30% to about 50%, about 30% to about 40%, about 40% to about 90%, about 40% to about 70%, about 40% to about 60%, about 40% to about 50%, about 50% to about 90%, about 50% to about 80%, about 50% to about 70%, about 50% to about 60%, or about 60% to about 80%. In some cases, the antibody disclosed herein inhibits activation of basophils by about 40% to about 60%. In some cases, the antibody disclosed herein inhibits activation of basophils by about 10%, 20%, 30%, 40%, 45%, 50%, 55%, 60%, 70%, 80%, or 90%. In some cases, the antibody disclosed herein inhibits activation of basophils by about 50% or about 55%. Activation of basophils can be measured by assessing the expression of CD63 by the basophils, for instance, by immunostaining of CD63, such as via fluorescent imaging and analysis or flow cytometry. Inhibition of basophil activation by CD200R antibody disclosed can be assessed by a basophil activation assay, such as the assay described in Example 10.

Binding Affinity

In some cases, the antibody provided herein binds a region at C-terminus of CD200R. In some cases, the antibody provided herein binds a region at C-terminus of the extracellular portion of CD200R. In some cases, the antibody provided herein binds a region at or in proximity of C-terminus of the extracellular portion of CD200R. In other cases, the antibody provided herein binds a region in proximity of N-terminus of CD200R. In some cases, the antibody provided herein binds a residue of CD200R selected from T213, E230, and S194. In some cases, the antibody provided herein binds a residue of CD200R selected from T213 and E230. In some cases, the antibody provided herein binds a T213 residue of CD200R. In some cases, the antibody provided herein binds a E230 residue of CD200R. In some cases, the antibody provided herein binds a T213 residue and a E230 residue of CD200R.

In some cases, the antibody provided herein specifically binds a region at C-terminus of the extracellular portion of CD200R. In some cases, the antibody provided herein binds a region at or in proximity of the extracellular portion of C-terminus of CD200R. In some cases, the antibody provided herein binds a region at most 50 amino acids, 45 amino acids, 40 amino acids, 35 amino acids, 30 amino acids, 25 amino acids, 20 amino acids, or 15 amino acids from the C-terminus of the extracellular portion of CD200R. In some cases, the antibody provided herein binds a region about 50 amino acids, 45 amino acids, 40 amino acids, 35 amino acids, 30 amino acids, 25 amino acids, 20 amino acids, or 15 amino acids from the C-terminus of the extracellular portion of CD200R. In some cases, the antibody or antigen-binding fragment thereof binds a region at most 100 Å, 90 Å, 80 A, 70 Å, 60 Å, 50 Å, 40 Å, 30 Å, 20 Å, or 10 A from the cell membrane when the antibody or antigen-binding fragment thereof binds to a CD200R molecule on the cell membrane. In some cases, the antibody or antigen-binding fragment thereof binds a region about 100 Å, 90 Å, 80 Å, 70 A, 60 Å, 50 Å, 40 Å, 30 Å, 20 Å, or 10 A from the cell membrane when the antibody or antigen-binding fragment thereof binds to a CD200R molecule on the cell membrane.

In other cases, the antibody provided herein specifically binds CD200R at a region in proximity of N-terminus of CD200R. In some cases, the antibody provided herein specifically binds CD200R at a residue of CD200R selected from T213, E230, and S194. In some cases, the antibody provided herein specifically binds CD200R at a residue of CD200R selected from T213 and E230. In some cases, the antibody provided herein specifically binds T213 residue of CD200R. In some cases, the antibody provided herein specifically binds E230 residue of CD200R. In some cases, the antibody provided herein specifically binds T213 residue and E230 residue of CD200R.

In some aspects, provided herein is an antibody that binds specifically to human CD200R or cynomolgus CD200R, but does not bind to cynomolgus CD200RLa, or binds to cynomolgus CD200RLa with a K_{D} of more than 2 μM, as determined by surface plasmon resonance (SPR) at 37° C. In some cases, the antibody described herein binds specifically to both human CD200R and cynomolgus CD200R.

CD200RLa can be an activating receptor that recognizes and binds to CD200. Without wishing to be bound by a certain theory, upon binding to CD200, CD200RLa expressed on the surface of an immune cell can lead to or promote activation of the immune cell. Cynomolgus CD200RLa can be highly homologous to cynomolgus CD200R. Lack of binding to cyno CD200RLa by an antibody that binds specifically to human or cynomolgus CD200R can be highly desirable because it can enable the use of cynomolgus monkey as model organisms for toxicological studies of the antibody.

Without wishing to be bound by a certain theory, epitopes of antibodies provided herein, e.g., clone 21.3.1, can have residue 230E of human CD200R. Sequence alignment of human CD200R, cynomolgus CD200R, and cynomolgus CD200RLa reveals that the glutamic acid at position 230 in human CD200R is also present in cynomolgus CD200R, but in cynomolgus CD200RLa the residue at the position corresponding to position 230 of human CD200R is lysine instead. The amino acid residual difference can have significant impact as glutamic acid is negatively charged whilst lysine is positively charged, which may be a molecular basis for the desirable lack of binding of antibodies disclosed herein (e.g., clone 21.3.1) to cynomolgus CD200RLa.

In some embodiments, the antibody described herein binds to cynomolgus CD200RLa with a K_{D} of more than 2 μM, as determined by surface plasmon resonance (SPR) at 37° C. In some embodiments, the antibody described herein binds to cynomolgus CD200RLa with a K_{D} of more than 5 μM, as determined by surface plasmon resonance (SPR) at 37° C. In some embodiments, the antibody described herein binds to cynomolgus CD200RLa with a K_{D} of more than 10 μM, as determined by surface plasmon resonance (SPR) at 37° C. In some embodiments, the antibody described herein binds to cynomolgus CD200RLa with a K_{D} of more than 100 μM, as determined by surface plasmon resonance (SPR) at 37° C. In some embodiments, the antibody described herein binds to cynomolgus CD200RLa with a K_{D} of more than 1 mM, as determined by surface plasmon resonance (SPR) at 37° C.

In some embodiments, the antibody described herein binds human CD200R with a K_{D} of less than 20 nM, as determined by surface plasmon resonance (SPR) at 37° C. In some embodiments, the antibody described herein binds human CD200R with a K_{D} of less than 10 nM, as determined by surface plasmon resonance (SPR) at 37° C. In some embodiments, the antibody described herein binds human CD200R with a K_{D} of less than 5 nM, as determined by surface plasmon resonance (SPR) at 37° C. In some embodiments, the antibody described herein binds human CD200R with a K_{D} of less than 3 nM, as determined by surface plasmon resonance (SPR) at 37° C. In some embodiments, the antibody described herein binds human CD200R with a K_{D} of less than 2 nM, as determined by surface plasmon resonance (SPR) at 37° C. In some embodiments, the antibody described herein binds human CD200R with a K_{D} of less than 1 nM, as determined by surface plasmon resonance (SPR) at 37° C. In some embodiments, the antibody described herein binds human CD200R with a K_{D} of less than 0.5 nM, as determined by surface plasmon resonance (SPR) at 37° C. In some embodiments, the antibody described herein binds human CD200R with a K_{D} of less than 0.1 nM, as determined by surface plasmon resonance (SPR) at 37° C.

In some embodiments, the antibody described herein binds cynomolgus CD200R with a K_{D} of less than 200 nM, as determined by surface plasmon resonance (SPR) at 37° C. In some embodiments, the antibody described herein binds cynomolgus CD200R with a K_{D} of less than 100 nM, as determined by surface plasmon resonance (SPR) at 37° C. In some embodiments, the antibody described herein binds cynomolgus CD200R with a K_{D} of less than 50 nM, as determined by surface plasmon resonance (SPR) at 37° C. In some embodiments, the antibody described herein binds cynomolgus CD200R with a K_{D} of less than 20 nM, as determined by surface plasmon resonance (SPR) at 37° C. In some embodiments, the antibody described herein binds cynomolgus CD200R with a K_{D} of less than 10 nM, as determined by surface plasmon resonance (SPR) at 37° C. In some embodiments, the antibody described herein binds cynomolgus CD200R with a K_{D} of less than 5 nM, as determined by surface plasmon resonance (SPR) at 37° C. In some embodiments, the antibody described herein binds cynomolgus CD200R with a K_{D} of less than 1 nM, as determined by surface plasmon resonance (SPR) at 37° C. In some embodiments, the antibody described herein binds cynomolgus CD200R with a K_{D} of less than 0.1 nM, as determined by surface plasmon resonance (SPR) at 37° C. In some embodiments, the antibody described herein binds cynomolgus CD200R with a K_{D} of less than 0.01 nM, as determined by surface plasmon resonance (SPR) at 37° C.

Antibody Engineering

An antibody embodied herein can be a monoclonal antibody, a chimeric antibody, a human or humanized antibody.

The subject antibody can be prepared by the hybridoma process or the recombinant DNA process. As described in the method of Kohler & Milstein (Nature, 256:495 (1975), antibody-producing cells used in the cell fusion step of preparing hybridomas are spleen cells, lymph node cells, peripheral blood leukocytes, etc. of an animal (e.g., mouse, rat, hamster, rabbit, monkey, goat) immunized with an antigen (human CD200R, its partial peptide, or cells expressing them). It is also possible to use antibody-producing cells obtained by allowing an antigen to act in a culture medium on the above cells or lymphocytes isolated in advance from an unimmunized animal. As myeloma cells, publicly known various cell strains can be used. The antibody-producing cells and myeloma cells may originate in different animal species, if they are mutually fusible; preferably, however, they are of the same animal species origin. Hybridomas, for example, are produced by cell fusion between spleen cells obtained from an antigen-immunized mouse and mouse myeloma cells, and subsequent screening can obtain hybridomas producing a monoclonal antibody against CD200R. The monoclonal antibody against CD200R can be produced by a culture of the hybridomas, or from an ascitic fluid of a mammal administered the hybridomas.

In some embodiments, the antibody disclosed herein is a humanized antibody. In making humanized antibodies, the choice of framework residues can be critical in retaining high binding affinity. In principle, a framework sequence from any HuAb can serve as the template for CDR grafting; however, it has been demonstrated that straight CDR replacement into such a framework can lead to significant loss of binding affinity to the antigen. Glaser et al. (1992) J. Immunol. 149:2606; Tempest et al. (1992) Biotechnology 9:266; and Shalaby et al. (1992) J. Exp. Med. 17:217. The more homologous a HuAb is to the original muAb, the less likely that the human framework will introduce distortions into the murine CDRs that could reduce affinity. Based on a sequence homology search against an antibody sequence database, the HuAb IC4 provides good framework homology to muM4TS.22, although other highly homologous HuAbs would be suitable as well, especially kappa L chains from human subgroup I or H chains from human subgroup III. Kabat et al. (1987). Various computer programs such as ENCAD (Levitt et al. (1983) J. Mol. Biol. 168:595) are available to predict the ideal sequence for the V region. The disclosure thus encompasses HuAbs with different V regions. It is within the skill of one in the art to determine suitable V region sequences and to optimize these sequences. Methods for obtaining antibodies with reduced immunogenicity are also described in U.S. Pat. No. 5,270,202 and EP 699,755.

In some embodiments, the antibody disclosed herein is selected from the group consisting of: scFv, sc(Fv)2, dsFv, Fab, Fab′, (Fab′)2 and a diabody. In some embodiments, the antibody disclosed herein is scFv. In some embodiments, the antibody disclosed herein is sc(Fv)2. In some embodiments, the antibody disclosed herein is dsFv. In some embodiments, the antibody disclosed herein is Fab. In some embodiments, the antibody disclosed herein is Fab′. In some embodiments, the antibody disclosed herein is (Fab′)2. In some embodiments, the antibody disclosed herein is a diabody.

In some embodiments, the antibody disclosed herein comprises an Fc region. In some embodiments, the Fc region comprises a modification. In some embodiments, the Fc region comprises one modification. In some embodiments, the Fc region comprises at least one modification. In some embodiments, the Fc region comprises two or more modifications.

In one aspect, the antibody disclosed herein comprises a heavy chain and a light chain, wherein the heavy chain comprises the heavy chain variable region operably linked to the Fc region, and wherein the light chain comprises the light chain variable region. In one feature, the antibody disclosed herein is a humanized antibody. In one aspect, the antibody disclosed herein is a human antibody. In another embodiment, the antibody disclosed herein is selected from the group consisting of: a human antibody, a humanized antibody, a chimeric antibody, and a multispecific antibody. In some cases, the antibody disclosed herein is a monoclonal antibody.

In some embodiments, the heavy chain or the light chain of the antibody disclosed herein further comprises a constant region. In some embodiments, the heavy chain or the light chain of the antibody disclosed herein are connected. In some embodiments, the heavy chain or the light chain of the antibody disclosed herein are connected by a linker. In some embodiments, the heavy chain or the light chain of the antibody disclosed herein are connected by a flexible linker. In some embodiments, the heavy chain or the light chain of the antibody disclosed herein are connected by a flexible linker to form a single-chain antibody. In some cases, the flexible linker is a peptide linker.

The peptide linker connecting scFv VH and VL domains joins the carboxyl terminus of one variable region domain to the amino terminus of another variable domain without significantly compromising the fidelity of the VH-VL pairing and antigen-binding sites. In some aspects, the peptide linkers are from 10 to 25 amino acids in length. In some cases, the peptide linkers are composed of hydrophilic amino acids such as glycine (G) and serine (S). In yet other cases, the peptide linkers are not composed of hydrophilic amino acids. In some embodiments, the linker is found in natural multi-domain proteins (e.g., see Argos P. J Mol Biol. 211:943-958, 1990; and. Heringa G. Protein Eng. 15:871-879, 2002), or adapted therefrom.

Commonly used flexible linkers have sequences consisting primarily of stretches of Gly and Ser residues (“GS” linker). An example of the most widely used flexible linker has the sequence of (Gly-Gly-Gly-Gly-Ser)ₙ. By adjusting the copy number “n”, the length of this GS linker can be altered to achieve appropriate separation of the functional domains, or to maintain necessary inter-domain interactions. Generally, the (GGGGS)3 peptide is used as an scFv peptide linker (Leith et al., Int. J. Oncol. 24:765-771, 2004; Holiger et al. Proc. Natl. Acad. Sci. U.S.A. 90:6444-6448, 1993). This 15-amino acid linker sequence [designated as the (GGGGS)3 linker] is used in the Recombinant Phage Antibody System (RPAS kit) commercially available from Amersham. Several other linkers have also been used to create scFV molecules (e.g., KESGSVSSEQLAQFRSLD and EGKSSGSGSESKST; Bird et al., Science 242:432-426, 1988).

Humanization

In some embodiments, the antibody provided herein is a monoclonal antibody. In some cases, the antibody provided herein is a monoclonal humanized antibody. In some embodiments, the antibody provided herein is a chimeric antibody. In some cases, the antibody provided herein is a chimeric humanized antibody. In some cases, the antibody provided herein is a bispecific antibody. In some cases, the antibody provided herein is a bispecific humanized antibody. In some cases, the antibody provided herein is a multispecific antibody. In some cases, the antibody provided herein is a multispecific humanized antibody. In some cases, the antibody disclosed herein comprises a monoclonal antibody. In some cases, the antibody disclosed herein comprises a monoclonal humanized antibody.

In some embodiments, provided herein are antibody variants comprising any potential combinations of humanized VH and VL domains. In some embodiment, the antibody provided herein comprises humanized variants of VH of CD200R antibody comprising human framework sequences. In some embodiments, the antibody or antigen-binding fragment comprises humanized variants of VL of CD200R antibody comprising human framework sequences.

Antibodies that are humanized can retain high affinity for the antigen and other favorable biological properties. To achieve this goal, in one example, CD200R humanized antibodies are prepared by a process of analysis of the parental sequences and various conceptual humanized products using three dimensional models of the parental and humanized sequences. Three dimensional immunoglobulin models are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, and of residues that influence the ability of the candidate immunoglobulin to bind its antigen. In this way, FR residues can be selected and combined from the consensus and import sequence so that the desired antibody characteristic, such as increased affinity for the target antigen(s), is achieved.

In some embodiments, the humanized VH chain comprises an amino acid sequence with at least 80%, 85%, 90%, 95%, 96%, 97%, 98%, 99%, or 100% sequence identity to the sequence set forth in SEQ ID NO: 42. In some embodiments, the humanized VH chain comprises an amino acid sequence with at least 80%, 85%, 90%, 95%, 96%, 97%, 98%, 99%, or 100% sequence identity to the sequence set forth in SEQ ID NO: 43. In some embodiments, the humanized VH chain comprises an amino acid sequence with at least 80%, 85%, 90%, 95%, 96%, 97%, 98%, 99%, or 100% sequence identity to the sequence set forth in SEQ ID NO: 44. In some embodiments, the humanized VH chain comprises an amino acid sequence with at least 80%, 85%, 90%, 95%, 96%, 97%, 98%, 99%, or 100% sequence identity to the sequence set forth in SEQ ID NO: 45. In some embodiments, the humanized VH chain comprises an amino acid sequence with at least 80%, 85%, 90%, 95%, 96%, 97%, 98%, 99%, or 100% sequence identity to the sequence set forth in SEQ ID NO: 46. In some embodiments, the humanized VH chain comprises an amino acid sequence with at least 80%, 85%, 90%, 95%, 96%, 97%, 98%, 99%, or 100% sequence identity to the sequence set forth in SEQ ID NO: 47.

In some cases, the variable light (VL) chain of the provided antibody comprises amino acid sequence set forth in SEQ ID NO: 2. In some embodiments, the humanized VL chain comprises an amino acid sequence with at least 80%, 85%, 90%, 95%, 96%, 97%, 98%, 99%, or 100% sequence identity to the sequence set forth in SEQ ID NO: 48. In some embodiments, the humanized VL chain comprises an amino acid sequence with at least 80%, 85%, 90%, 95%, 96%, 97%, 98%, 99%, or 100% sequence identity to the sequence set forth in SEQ ID NO: 49. In some embodiments, the humanized VL chain comprises an amino acid sequence with at least 80%, 85%, 90%, 95%, 96%, 97%, 98%, 99%, or 100% sequence identity to the sequence set forth in SEQ ID NO: 50. In some embodiments, the humanized VL chain comprises an amino acid sequence with at least 80%, 85%, 90%, 95%, 96%, 97%, 98%, 99%, or 100% sequence identity to the sequence set forth in SEQ ID NO: 51. In some embodiments, the humanized VL chain comprises an amino acid sequence with at least 80%, 85%, 90%, 95%, 96%, 97%, 98%, 99%, or 100% sequence identity to the sequence set forth in SEQ ID NO: 52. In some embodiments, the humanized VL chain comprises an amino acid sequence with at least 80%, 85%, 90%, 95%, 96%, 97%, 98%, 99%, or 100% sequence identity to the sequence set forth in SEQ ID NO: 65. In some embodiments, the humanized VL chain comprises an amino acid sequence with at least 80%, 85%, 90%, 95%, 96%, 97%, 98%, 99%, or 100% sequence identity to the sequence set forth in SEQ ID NO: 66.

In some case, the humanized CDRH1 of the antibody provided herein comprises an amino acid sequence with at least 80%, 85%, 90%, 95%, 96%, 97%, 98%, 99%, or 100% sequence identity to the sequence set forth in SEQ ID NO: 3, 11, 19, 27, or 35. In some case, the humanized CDRH2 of the antibody comprises an amino acid sequence with at least 80%, 85%, 90%, 95%, 96%, 97%, 98%, 99%, or 100% sequence identity to the sequence set forth in SEQ ID NO: 4, 41, 12, 20, 28, or 36. In some case, the humanized CDRH3 of the antibody comprises an amino acid sequence with at least 80%, 85%, 90%, 95%, 96%, 97%, 98%, 99%, or 100% sequence identity to the sequence set forth in SEQ ID NO: 5, 13, 21, 29, or 37.

In some cases, the humanized CDRH1 of the antibody provided herein comprises the sequence set forth in SEQ ID NO: 3, 11, 19, 27, or 35, with 0 to 3 amino acid modifications, such as 0, 1, 2, or 3 modifications. In some cases, the humanized CDRH2 of the antibody comprises the sequence set forth in SEQ ID NO: 4, 41, 12, 20, 28, or 36, with 0 to 3 amino acid modifications, such as 0, 1, 2, or 3 modifications. In some case, the humanized CDRH3 of the antibody comprises the sequence set forth in SEQ ID NO: 5, 13, 21, 29, or 37, with 0 to 3 amino acid modifications, such as 0, 1, 2, or 3 modifications.

In some case, the humanized CDRL1 of the antibody provided herein comprises an amino acid sequence with at least 80%, 85%, 90%, 95%, 96%, 97%, 98%, 99%, or 100% sequence identity to the sequence set forth in SEQ ID NO: 6, 14, 22, 30, or 38. In some case, the humanized CDRL2 of the antibody comprises an amino acid sequence with at least 80%, 85%, 90%, 95%, 96%, 97%, 98%, 99%, or 100% sequence identity to the sequence set forth in SEQ ID NO: 7, 15, 23, 31, or 39. In some case, the humanized CDRL3 of the antibody comprises an amino acid sequence with at least 80%, 85%, 90%, 95%, 96%, 97%, 98%, 99%, or 100% sequence identity to the sequence set forth in SEQ ID NO: 8, 16, 24, 32, or 40.

In some cases, the humanized CDRL1 of the antibody provided herein comprises the sequence set forth in SEQ ID NO: 6, 14, 22, 30, or 38, with 0 to 3 amino acid modifications, such as 0, 1, 2, or 3 modifications. In some cases, the humanized CDRL2 of the antibody comprises the sequence set forth in SEQ ID NO: 7, 15, 23, 31, or 39, with 0 to 3 amino acid modifications, such as 0, 1, 2, or 3 modifications. In some case, the humanized CDRL3 of the antibody comprises the sequence set forth in SEQ ID NO: 8, 16, 24, 32, or 40, with 0 to 3 amino acid modifications, such as 0, 1, 2, or 3 modifications.

Modfications

In some embodiments provided herein, the CD200R antibody as described herein may have one or more mutations or modifications with respect to a reference sequence. A mutation or modification may be a deletion, an insertion or addition, or a replacement or substitution to an amino acid residue. A “deletion” refers to a change in an amino acid sequence due to the absence of one or more amino acid residues. An “insertion” or “addition” refers to changes in an amino acid sequence resulting in the addition of one or more amino acid residues as compared to a reference sequence. A “replacement” or “substitution” refers to the replacement of one or more amino acids by different amino acids. In the context of the present disclosure, the mutations of a subject antibody or a fraction thereof with respect to a reference sequence may be determined by comparison of the subject antibody or a fraction thereof to the reference sequence. Optimal alignment of sequences for comparison may be conducted according to any of the known methods in the art.

A mutation may be identified by the mutation site. The mutation site is the position on a reference sequence where a modification, such as a deletion, an addition, or a substitution, takes place. The amino acid residues on a reference sequence are numbered from the N-terminus to the C-terminus, and the mutation site is the numbering of the amino acid residue on which a deletion, an addition, or a substitution takes place. For example, position 26 on a reference sequence is the position where the 26^{th} amino acid residue locates starting from the N-terminus.

In some cases, the antibody or antigen-binding fragment thereof disclosed herein comprises a heavy chain and a light chain, wherein said heavy chain comprises an Fc region that comprises an arginine at position 330 (EU Index). In some cases, the antibody or antigen-binding fragment thereof disclosed herein comprises a heavy chain and a light chain, wherein said heavy chain comprises an Fc region that comprises an aspartic acid at position 237 (EU Index), an aspartic acid at position 238 (EU Index), a glycine at position 271 (EU Index) and an arginine at position 330 (EU Index). Suitably the antibody or antigen-binding fragment thereof disclosed herein can be an agonistic antibody/antigen-binding fragment.

In some cases, the antibody or antigen-binding fragment thereof disclosed herein comprises an Fc region that comprises one or more of the following amino acids: alanine (A) at position 234, alanine (A) at position 235, aspartic acid (D) at position 236, aspartic acid (D) at position 237, aspartic acid (D) at position 238, alanine (A) at position 265, glutamic acid (E) at position 267, glycine (G) at position 271, arginine (R) at position 330, alanine (A) at position 332, and alanine (A) at position 297 (all numbering according to EU Index).

In a particular embodiment, the antibody or antigen-binding fragment thereof disclosed herein comprises an Fc region (e.g., Fc of IgGI) that comprises an aspartic acid at position 236 (EU Index). Suitably the antibody is an agonistic antibody.

In a particular embodiment, the antibody or antigen-binding fragment thereof disclosed herein comprises an Fc region (e.g., Fc of IgGI) that comprises an aspartic acid at position 237 (EU Index). Suitably the antibody is an agonistic antibody.

In a particular embodiment, the antibody or antigen-binding fragment thereof disclosed herein comprises an Fc region (e.g., Fc of IgGI) that comprises an aspartic acid at position 238 (EU Index). Suitably the antibody is an agonistic antibody.

In a particular embodiment, the antibody or antigen-binding fragment thereof disclosed herein comprises an Fc region (e.g., Fc of IgGI) that comprises an alanine at position 235 (EU Index).

In a particular embodiment, the antibody or antigen-binding fragment thereof disclosed herein comprises an Fc region (e.g., Fe of IgGI) that comprises an alanine at position 234 (EU Index).

In a particular embodiment, the antibody or antigen-binding fragment thereof disclosed herein comprises an Fc region (e.g., Fe of IgGI) that comprises an alanine at position 265 (EU Index).

In a particular embodiment, the antibody or antigen-binding fragment thereof disclosed herein comprises an Fc region (e.g., Fe of IgGI) that comprises a glutamic acid at position 267 (EU Index).

In a particular embodiment, the antibody or antigen-binding fragment thereof disclosed herein comprises an Fc region (e.g., Fe of IgGI) that comprises a glycine at position 271 (EU Index).

In a particular embodiment, the antibody or antigen-binding fragment thereof disclosed herein comprises an Fc region (e.g., Fe of IgGI) that comprises an alanine at position 297 (EU Index).

In a particular embodiment, the antibody or antigen-binding fragment thereof disclosed herein comprises an Fc region (e.g., Fe of IgGI) that comprises an alanine at position 322 (EU Index).

In a particular embodiment, the antibody or antigen-binding fragment thereof disclosed herein comprises an Fc region (e.g., Fe of IgGI) that comprises an arginine at position 330 (EU Index).

In a particular embodiment, the antibody or antigen-binding fragment thereof disclosed herein comprises an Fc region (e.g., Fe of IgGI) that comprises an aspartic acid at position 237 (EU Index), an aspartic acid at position 238 (EU Index), a glycine at position 271 (EU Index) and an arginine at position 330 (EU Index).

In some embodiments, the antibody or antigen-binding fragment thereof of is an IgGI antibody or comprises an Fc region of an IgGI antibody. In some embodiments, the IgGI antibody comprises one or more modifications at positions 234, 235, 236, 238, 239, 243, 250, 252, 254, 256, 257, 292, 297, 311, 322, 326, 329, 330, 332, 333, 396, 428, 433, and 434 (EU index). In some embodiments, the IgGI antibody comprises one or more modifications selected from L234 Å, L235 Å, L235V, G236 Å, P238D; S239D, F243L, T250Q, M252Y, S254T, T256E, P2571, R292P, N297D, Q311, K322 Å, K326W, P329 Å, P329G, A330L, 1332E, E333 Å, E333S, P396L, M428L, H433K, and N434F (EU index). In some embodiments, the IgGI antibody comprises one or more modifications selected from: (a) S239D, A330L, and I332E (EU index); (b) L234A and L235A (EU index); (c) T250Q and M428L (EU index); (d) M252Y, S254T, T256E, H433K, and N434F (EU index); (e) E333A (EU index); (f) P2571 and Q311 (EU index); (g) K326W and E333S (EU index); (h) S239D, I332E, and G236A (EU index); (i) K322A (EU index); and (j) P238D (EU index). In some embodiments, IgGI antibody comprises a P238D substitution.

In some embodiments, the antibody or antigen-binding fragment thereof of is an IgG2 antibody or comprises an Fc region of an IgG2 antibody. In some embodiments, the IgG2 antibody is derived from a mouse IgG2 antibody. In some embodiments, the mouse IgG2 antibody comprises one or more modifications selected from L235E, E318 Å, K320 Å, and K322A (EU index).

In some embodiments, the antibody or antigen-binding fragment thereof of is an IgG3 antibody or comprises an Fc region of an IgG3 antibody.

In some embodiments, the antibody or antigen-binding fragment thereof of is an IgG4 antibody or comprises an Fc region of an IgG4 antibody. In some embodiments, the IgG4 antibody comprises one or more modifications at positions 228, 234, 235, 327, 329, 330 and 331 (EU index).

In some embodiments, the IgG4 antibody comprises one or more modifications selected from S228P, L234F, L235E, A327G, P329G, A330S and P331S (EU index). In some embodiments, the IgG4 antibody comprises a S228P substitution (EU index).

In some cases, the antibody or antigen-binding fragment thereof disclosed herein possesses increased binding to FcγR2B compared to the parent molecule that lacks the Fc region substitution, i.e., one or more of: hIgGI G236D, hIgGI G237D, hIgGI P238D, hIgGI D265 Å, hIgGI S267E, hIgGI P271G, hIgGI A330R, hIgGI K322 Å, hIgGI N297 Å, hIgG4 P238D, hIgG4 G237D, hIgG4 P271G, hIgG4 S330R, hIgG4 F234 Å, or hIgG4 L235A.

In some cases, the antibody or antigen-binding fragment thereof disclosed herein possesses increased binding to FcγR2B and reduced binding to one or more activating Fc7 receptors, such as FcγR2A (e.g., 131R allotype or 131H allotype) or FcγR1 Å, compared to the parent molecule that lacks the Fc region substitution.

In some cases, the antibody or antigen-binding fragment thereof disclosed herein possesses increased ratio of binding to FcγR2B/FcγR2A (e.g., 131R allotype or 131H allotype), compared to the parent molecule that lacks the Fc region substitution. Suitably, the increased ratio of binding FcγR2B/FcγR2A (e.g., 131R allotype or 131H allotype), is at least 1.1, 1.2, 1.3, 1.4, 1.5, 1.8, 2, 2.2, 2.5, 3, 3.5, 4, 5, 6, 7, 8, 9, 10, 15, 20,25, 30, 40, 50, 60, 70, 80, 90, 100, 110, 120, 130, 140, or 150-fold compared to the parent molecule that lacks the Fc region substitution.

In some cases, the Fc region of the antibody or antigen-binding fragment thereof disclosed herein binds to FcγR2B with a higher affinity relative to a comparable control antibody that comprises an Fc region that lacks the one or more Fc substitutions recited above. In some cases, the antibody binds to FcγR2B with a dissociation constant (K_{D}) of from about 5 μM to 0.1p M, as determined by surface plasmon resonance (SPR). Suitably, the antibody binds to FcγR2B via its Fc region.

In some cases, the antibody binds to FcγR2B with a K_{D} of at most 5 μM, as determined by surface plasmon resonance (SPR).

In some cases, the antibody binds to FcγR2A (131R allotype) with a lower or equal affinity relative to a parental molecule. A parental molecule being the equivalent antibody that lacks the Fc substitution that confers on the antibody molecule an increased binding to and thus enhanced signaling of FcγR2B.

In some cases, when the antibody comprises the P238D substitution the antibody binds to FcγR2A (131R allotype) with a lower or equal affinity relative to a comparable control antibody that comprises an Fc region that comprises a proline at position 238 (EU Index).

In some cases, the antibody binds to FcγR2A (131R allotype) with a K_{D} of at least 20 μM, as determined by surface plasmon resonance (SPR).

In some cases, the antibody binds to FcγR2A (131R allotype) with a K_{D} of from about 25 μM to 35 μM, as determined by surface plasmon resonance (SPR).

In some cases, the antibody binds to FcγR2A (131H allotype) with a lower or equal affinity relative to a parental molecule.

In some cases, the antibody binds to FcγR2A (131H allotype) with a K_{D} of at least 50 μM, as determined by surface plasmon resonance (SPR).

In some cases, the antibody possesses a [K_{D} value of the antibody for FcγR2A (131R) /K_{D} value of the antibody for FcγR2B] of 3 or more, such as at least 5. Suitably, as determined by surface plasmon resonance (SPR).

In some cases, the antibody possesses a [K_{D} value of the antibody for FcγR2A(131H)]/[K_{D} value of the antibody for FcγR2B] of 10 or more, such as at least 15. Suitably, as determined by surface plasmon resonance (SPR).

In some cases, the antibody possesses a [K_{D} value of the antibody for FcγR2A (131R) /K_{D} value of the antibody for FcγR2B] of 3 or more, such as at least 5 and/or a [K_{D} value of the antibody for FcγR2A(131H)]/[K_{D} value of the antibody for FcγR2B] of 10 or more, such as at least 15. Suitably, as determined by surface plasmon resonance (SPR).

In some cases, the antibody or antigen-binding fragment thereof disclosed herein possesses increased ratio of binding to FcγR2B/FcγR1 Å, compared to the parent molecule that lacks the Fc region substitution over the wild-type sequence. Suitably, the increased ratio of binding FcγR2B/FcγR1 Å, is at least 1.1, 1.2, 1.5, 2, 5, 10, 50, 100, 150, 200, 250-fold compared to the parent molecule that lacks the Fc region substitution.

By compared to the parent molecule that lacks the Fc region substitution it is meant compared to the antibody molecule that has the same amino acid sequence other than the amino acid recited in the claim which represents the Fc substitution relative to wildtype Fc. For example, including any one or more of the following substitutions: hIgGI G236D, hIgGI G237D, hIgGI P238D, hIgGI D265 Å, hIgGI S267E, hIgGI P271G, hIgGI A330R, hIgGI K322 Å, hIgGI N297 Å, hIgG4 P238D, hIgG4 G237D, hIgG4 P271G, hIgG4 S330R, hIgG4 F234 Å, or hIgG4 L235A. Thus, binding of the antibody molecule with or without the recited Fc substitution to FcγR2B can be measured and optionally binding of the antibody molecule with or without the recited Fc substitution to an activating Fc7 receptor, such as FcγR2A (e.g., 131R allotype or 131H allotype) or FcγR1A can be measured.

In some cases, the antibody or antigen-binding fragment thereof disclosed herein has an increased ratio of [K_{D} value for binding of FcγR1A]/[K_{D} value for binding of FcγR2B] compared to the parent molecule that lacks the Fc region substitution over the wild-type sequence. Suitably, the ratio of [K_{D} value for binding of FcγR1A]/[K_{D} value for binding of FcγR2B] for the variant molecule is at least 1.1, 1.2, 1.5, 2, 5, 10, 50, 100, 150, 200, 250, 300, 350, 400, 450, 500, 1000, 1500, 2000, 3000, 4000, 5000, 6000, 7000, 8000, 9000, or 10000 times the ratio of [K_{D} value for binding of FcγR1A]/[K_{D} value for binding of FcγR2B] for the parent molecule that lacks the Fc region substitution.

In some cases, the antibody or antigen-binding fragment thereof disclosed herein has an increased ratio of [K_{D} value for binding of FcγR2A (131R)]/[K_{D} value for binding of FcγR2B] compared to the parent molecule that lacks the Fc region substitution over the wild-type sequence. Suitably, the ratio of [K_{D} value for binding of FcγR2A (131R)]/[K_{D} value for binding of FcγR2B] for the variant molecule is at least 1.1, 1.2, 1.5, 2, 5, 10, 50, or 100 times the ratio of [K_{D} value for binding of FcγR1A]/[K_{D} value for binding of FcγR2B] for the parent molecule that lacks the Fc region substitution.

In some cases, the antibody provided herein that specifically binds CD200R comprises a light chain containing a light chain variable region that incorporates an amino acid change at position 56 from an Aspartate to either a Serine or Threonine when numbered according to SEQ ID NO: 2, 26, 34, 48, 49, 50, 51, or 52. In some cases, the antibody provided herein that specifically binds CD200R comprises a light chain containing a light chain variable region that comprises the sequence of SEQ ID NO: 2, 26, 34, 48, 49, 50, 51, or 52, and an amino acid modification from an Aspartate to either a Serine or Threonine at position 56, when numbered according to SEQ ID NO: 2, 26, 34, 48, 49, 50, 51, or 52. Without wishing to be bound to a certain theory, amino acid at position 56, when numbered according to SEQ ID NO: 2, is part of CDRL2 region, and Aspartate at position 56 can confer a deamidation risk, which is removed by substitution for Serine or Threonine. Without wishing to be bound to a certain theory, the replacement of Aspartate by Serine or Threonine at position 56 can reduce deamidation risk, while maintaining a human-like, non-immunogenic sequence, as well as affinity of the antibody to CD200R. In some cases, the antibody provided herein that specifically binds CD200R comprises a light chain containing a light chain variable region that comprises the sequence of SEQ ID NO: 65 or 66.

Antibody Conjugates

In some embodiments, an antibody disclosed herein is conjugated with an agent forming an immunoconjugate. In some embodiments, an antibody disclosed herein is fused to serum albumins. Fusion to serum albumins can improve the pharmacokinetics of a subject antibody as described herein. For example, the subject antibody or fragment thereof may be fused with a serum albumin. Serum albumin is a globular protein that is the most abundant blood protein in mammals. Serum albumin is produced in the liver and constitutes about half of the blood serum proteins. It is monomeric and soluble in the blood. In some embodiments, the subject antibody or fragment thereof may be fused to a serum albumin. In further embodiments, serum albumin is human serum albumin (HSA).

In some embodiments, an antibody or fragment thereof disclosed herein is fused to an albumin-binding peptide that displays binding activity to serum albumin to increase the half-life of the subject antibody or fragment thereof. Albumin-binding peptides that can be used herein include but are not limited to those described in e.g., Dennis et al., J. Biol. Chem. 277:35035-35043, 2002 and Miyakawa et al., J. Pharm. Sci. 102:3110-3118, 2013. In some embodiments, an albumin-binding peptide is fused genetically to a subject antibody or fragment thereof described herein. In further embodiments, an albumin-binding peptide is attached to a subject antibody described herein or fragment thereof through chemical means, e.g., chemical conjugation. In some embodiments, an albumin-binding peptide may be fused to the N- or C-terminus of a subject antibody or fragment thereof described herein. The C-terminus of the albumin-binding peptide may be directly fused to the N-terminus of the subject antibody through a peptide bond. Alternatively, the N-terminus of the albumin-binding peptide may be directly fused to the C-terminus of the subject antibody or fragment thereof through a peptide bond. In further embodiments, the carboxylic acid at the C-terminus of the albumin-binding peptide may be fused to an internal amino acid residue of the subject antibody or fragment thereof using conventional chemical conjugation techniques.

In some embodiments, a CD200R antibody or fragment thereof disclosed herein is fused to a polymer, e.g., polyethylene glycol (PEG). The antibody or fragment thereof can be pegylated to, for example, increase the biological (e.g., serum) half-life of the antibody or fragment thereof. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. Methods for pegylating proteins such as those disclosed in for example, EP 0 154 316 by Nishimura et al. and EP 0 401 384 by Ishikawa et al may be used. In some embodiments, a polymer, e.g., PEG, may be covalently attached to a subject antibody, or fragment thereof, described herein, either at the N- or C-terminus or at an internal location, using conventional chemical methods, e.g., chemical conjugation. Without being bound by a theory, PEG moieties may contribute to, once attached to the antibody as described herein, the water solubility, high mobility in solution, lack of toxicity and low immunogenicity, extended circulating life, increased stability, ready clearance from the body, and altered distribution in the body.

Other half-life extension technologies that may be used to increase the serum half-life of the subject antibodies, or fragment thereof, include, but are not limited to, XTEN (Schellenberger et al., Nat. Biotechnol. 27:1186-1192, 2009) and Albu tag (Trussel et al., Bioconjug Chem. 20:2286-2292, 2009).

In some embodiments, a CD200R antibody or fragment thereof disclosed herein is conjugated to a chemically functional moiety. Typically, the moiety is a label capable of producing a detectable signal. These conjugated antibodies or fragments thereof are useful, for example, in detection systems such as quantitation of tumor burden, and imaging of metastatic foci and tumor imaging. Such labels are known in the art and include, but are not limited to, radioisotopes, enzymes, fluorescent compounds, chemiluminescent compounds, bioluminescent compounds substrate cofactors and inhibitors. See, for examples of patents describing the use of such labels, U.S. Pat. Nos. 3,817,837; 3,850,752; 3,939,350; 3,996,345; 4,277,437; 4,275,149; and 4,366,241. The moieties can be covalently linked to antibody or fragment thereof as described herein, recombinantly linked, or conjugated to an antibody or fragment thereof through a secondary reagent, such as a second antibody, protein A, or a biotin-avidin complex.

Other functional moieties include signal peptides, agents that enhance or reduce immunologic reactivity, agents that facilitate coupling to a solid support, vaccine carriers, bioresponse modifiers, paramagnetic labels and drugs. A signal peptide is a short amino acid sequence that directs a newly synthesized protein through a cellular membrane, usually the endoplasmic reticulum in eukaryotic cells, and either the inner membrane or both inner and outer membranes of bacteria. Signal peptides are typically at the N-terminal portion of a polypeptide and are typically removed enzymatically between biosynthesis and secretion of the polypeptide from the cell. Such a peptide can be incorporated into the subject antibody or fragment thereof to allow secretion of the synthesized molecules.

Agents that enhance immunologic reactivity include, but are not limited to, bacterial superantigens. Agents that facilitate coupling to a solid support include, but are not limited to, biotin or avidin. Immunogen carriers include, but are not limited to, any physiologically acceptable buffers. Bioresponse modifiers include cytokines, particularly tumor necrosis factor (TNF), interleukin-2, interleukin-4, granulocyte macrophage colony stimulating factor and gamma-interferons.

Agents that reduce immunologic reactivity include, but are not limited to, anti-inflammatory agents and immunosuppressants. Anti-inflammatory agents include non-steroidal anti-inflammatory drugs (NSAIDs) and corticosteroids. NSAIDs include but are not limited to, salicylates, such as acetylsalicylic acid; diflunisal, salicylic acid, and salsalate; propionic acid derivatives, such as ibuprofen; naproxen; dexibuprofen, dexketoprofen, flurbiprofen, oxaprozin, fenoprofen, loxoprofen, and ketoprofen; acetic acid derivatives, such as indomethacin, diclofenac, tolmetin, aceclofenac, sulindac, nabumetone, etodolac, and ketorolac; enolic acid derivatives, such as piroxicam, lornoxicam, meloxicam, isoxicam, tenoxicam, phenylbutazone, and droxicam; anthranilic acid derivatives, such as mefenamic acid, flufenamic acid, meclofenamic acid, and tolfenamic acid; selective COX-2 inhibitors, such as celecoxib, lumiracoxib, rofecoxib, etoricoxib, valdecoxib, firocoxib, and parecoxib; sulfonanilides, such as nimesulide; and others such as clonixin, and licofelone. Corticosteroids include but are not limited to, cortisone, dexamethasone, hydrocortisone, methylprednisolone, prednisone, and prednisolone. The immunosuppressants include but are not limited to hydroxychloroquine, sulfasalazine, leflunomide, etanercept, infliximab, adalimumab, D-penicillamine, oral gold compound, injectable gold compound (intramuscular injection), minocycline, sodium gold thiomalate, auranofin, D-penicillamine, lobenzarit, bucillamine, actarit, cyclophosphamide, azathioprine, methotrexate, mizoribine, cyclosporine, and tacrolimus.

Suitable drug moieties include antineoplastic agents. Non-limiting examples are radioisotopes, vinca alkaloids such as the vinblastine, vincristine and vindesine sulfates, adriamycin, bleomycin sulfate, carboplatin, cisplatin, cyclophosphamide, cytarabine, dacarbazine, dactinomycin, duanorubicin hydrochloride, doxorubicin hydrochloride, etoposide, fluorouracil, lomustine, mechlororethamine hydrochloride, melphalan, mercaptopurine, methotrexate, mitomycin, mitotane, pentostatin, pipobroman, procarbaze hydrochloride, streptozotocin, taxol, thioguanine, and uracil mustard.

Immunotoxins, including single chain molecules, can be produced by recombinant means. A variety of immunotoxins are available, and methods can be found, for example, in Monoclonal Antibody-toxin Conjugates: Aiming the Magic Bullet, Thorpe et al. (1982) Monoclonal Antibodies in Clinical Medicine, Academic Press, pp. 168-190; Vitatta (1987) Science 238:1098-1104; and Winter and Milstein (1991) Nature 349:293-299. Suitable toxins include, but are not limited to, ricin, radionuclides, pokeweed antiviral protein, Pseudomonas exotoxin A, diphtheria toxin, ricin A chain, fungal toxins such as restrictocin and phospholipase enzymes. See, generally, “Chimeric Toxins,” Olsnes and Pihl, Pharmac. Ther. 15:355-381 (1981); and “Monoclonal Antibodies for Cancer Detection and Therapy,” eds. Baldwin and Byers, pp. 159-179, 224-266, Academic Press (1985).

The chemically functional moieties can be made recombinantly for instance by creating a fusion gene encoding the antibody and the functional moiety. Alternatively, the antibody or fragment thereof can be chemically bonded to the moiety by any of a variety of well-established chemical procedures. For example, when the moiety is a protein, a variety of coupling agents may be used such as N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCl), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). The linker may be a “cleavable linker” facilitating release of the cytotoxic drug in the cell. For example, an acid-labile linker, peptidase-sensitive linker, dimethyl linker, or disulfide-containing linker (Chari et al. Cancer Research, 52: 127-131 (1992)) may be used. The moieties may be covalently linked, or conjugated, through a secondary reagent, such as a second antibody, protein A, or a biotin-avidin complex. For examples of paramagnetic moieties and the conjugation thereof to antibodies, see, e.g., Miltenyi et al. (1990) Cytometry 11:231-238.

In some embodiments, a CD200R antibody or fragment thereof disclosed herein is a bispecific antibody. Bispecific antibodies are antibodies that have binding specificities for at least two different epitopes. A bispecific antibody as described herein may be a bispecific antibody that recognizes different epitopes on CD200R, or a bispecific antibody in which one of the antigen-binding sites recognizes CD200R and the other antigen-binding site recognizes an antigen other than CD200R.

Nucleic Acid Molecules

In some embodiments, the antibody described herein is encoded by one or more nucleic acid molecules. In one case, the antibody is encoded by a single nucleic acid molecule. In other cases, the antibody is encoded by two or more nucleic acid molecules. For example, as the antigen binding site is formed by the coming together of a heavy chain variable polypeptide region and a light chain variable polypeptide region, the two variable (heavy and light) polypeptide regions are encoded by separate nucleic acid molecules. Alternatively, for example, in the case of an ScFv, they are encoded by the same nucleic acid molecule.

According to some aspects of the disclosure there is provided one or more nucleic acid molecules that encode an antibody or antigen-binding fragment thereof in accordance with some embodiments of the present disclosure.

From the primary amino acid sequence of the polypeptide(s) encoding the antibody provided herein, the person of skill in the art is able to determine suitable nucleotide sequence(s) that encodes the polypeptide(s) and, if desired, one that is codon-optimized (e.g., see Mauro and Chappell. Trends Mol Med. 20(11):604-613, 2014).

According to some aspects of the disclosure there is provided an isolated nucleic acid comprising a nucleotide sequence that encodes a heavy chain variable region polypeptide or a light chain variable region polypeptide of the disclosure. A heavy chain variable polypeptide or a light chain variable polypeptide of the disclosure refers to the individual polypeptide chains that include amino acids that make up part of the antigen-binding site. In some cases, the polypeptides also comprise other domains such as constant domains, hinge regions, and an Fc region, such as one comprising one or more Fc receptor binding sites.

According to some aspects of the disclosure there is provided an isolated nucleic acid which comprises one or more nucleotide sequence encoding polypeptides capable of forming an antibody or antigen-binding fragment of the disclosure. In particular embodiments, the polypeptides may also comprise other domains such as constant domains, hinge regions, and an Fc region, such as one comprising one or more Fc receptor binding sites.

In one case, nucleic acid molecules encode just the polypeptide sequence that comprises the VL domain of the antibody or fragment thereof In some cases, encode just the polypeptide sequence that comprises the VH domain of the antibody or fragment thereof In other cases, the nucleic acid molecule encodes both VH and VL domain containing polypeptide sequences capable of forming the antibody or antibody fragment thereof of the disclosure.

The nucleic acid molecule(s) that encode the antibody or antigen-binding fragment thereof of the disclosure may be, or may be part of, a vector (such as a plasmid vector, cosmid vector or viral vector, or an artificial chromosome) that may comprise other functional regions (elements) such as one or more promoters, one or more origins or replication, one or more selectable marker(s), and one or more other elements typically found in expression vectors. The cloning and expression of nucleic acids that encode proteins, including antibodies, is well established and well within the skill of the person in the art.

Vectors

According to some aspects of the disclosure there is provided a vector comprising the nucleic acid according to some embodiments of the disclosure. In particular embodiments, the vector is a plasmid vector, cosmid vector, viral vector, or an artificial chromosome.

The nucleic acids of the present disclosure, including vector nucleic acids that comprise nucleotide sequences that encode the polypeptides capable of forming an antibody of the disclosure or an antigen-binding fragments thereof, may be in purified/isolated form.

Isolated/purified nucleic acids that encode an antibody or antigen-binding fragment thereof of the disclosure will be free or substantially free of material with which they are naturally associated, such as other proteins or nucleic acids with which they are found in their natural environment, or the environment in which they are prepared (e.g. cell culture) when such preparation is by recombinant DNA technology practised in vitro or in vivo.

In some embodiments, the nucleic acids of the disclosure are greater than 80%, such as greater than 90%, greater than 95%, greater than 97% and greater than 99% pure.

Thus, according to some aspects of the disclosure there is provided a vector comprising a nucleic acid or nucleotide sequence that encodes a heavy chain variable polypeptide or a light chain variable polypeptide of the disclosure. In a particular embodiment, the vector comprises nucleic acid that encodes both the heavy and light chain variable regions. In some embodiments, the polypeptides comprise other domains such as constant domains, hinge regions, and an Fc region, such as one comprising one or more Fc receptor binding sites.

In some embodiments, the nucleic acid and/or vector of the disclosure is introduced into a host cell. For eukaryotic cells, for example, suitable techniques include calcium phosphate transfection, DEAE-Dextran, electroporation, liposome-mediated transfection and transduction using retrovirus or other virus, e.g., vaccinia or, for insect cells, baculovirus. In one aspect, introducing nucleic acid in the host cell, in particular a eukaryotic cell, uses a viral or a plasmid-based system. In some cases, the plasmid system is maintained episomally. In other cases, the plasmid system is incorporated into the host cell or into an artificial chromosome. In a particular embodiment, the incorporation is by random integration of one or more copies at single or multiple loci. In some embodiments, the incorporation is by targeted integration of one or more copies at single or multiple loci. For bacterial cells, suitable techniques include, for example, calcium chloride transformation, electroporation and transfection using bacteriophage.

In one embodiment, the nucleic acid of the disclosure is integrated into the genome (e.g., chromosome) of the host cell. In a particular embodiment, integration is promoted by inclusion of sequences that promote recombination with the genome, in accordance with standard techniques.

Host Cells

A further aspect of the present disclosure provides a host cell containing nucleic acid as disclosed herein. In some embodiments, such a host cell is in vitro. In some embodiments, such a host cell is in culture.

In some cases, the host cell is from any species, such as a bacterium or yeast. In other cases, the host cell is a mammalian cell such as a human cell or rodent cell, for example a HEK293T cell or CHO-K1 cell.

Thus, according to some aspects of the disclosure there is provided a host cell comprising the nucleic acid sequence or the vector according to some embodiments of the present disclosure.

In some cases, the host cell is treated so as to cause or allow expression of the protein of the disclosure from the nucleic acid, e.g., by culturing host cells under conditions for expression of the encoding nucleic acid. In some embodiments, the purification of the expressed product is achieved by methods known to one of skill in the art.

In some embodiments, the nucleic acids of the disclosure, including vector nucleic acids that comprise nucleotide sequences that encode the polypeptides for the antibodies of the disclosure or antigen-binding fragments thereof, is present in an isolated host cell. In some cases, the host cell is part of a clonal population of host cells. As used herein, reference to a host cell also encompasses a clonal population of the cell. A clonal population is one that has been grown from a single parent host cell. In some cases, the host cell is from any suitable organism. In some cases, the host cell is, for example, bacterial, fungal or mammalian cells.

In some embodiments, the host cell assists in amplifying the vector nucleic acid (such as with a plasmid). In a particular embodiment, the host cell serves as the biological factory to express the polypeptide(s) of the disclosure that form the CR200R antibody described herein. In one case, a suitable host for amplifying the vector nucleic acid is a bacterial or fungal cell, such as an Escherichia coli cell or Saccharomyces cerevisiae cell. In other cases, a suitable host for expressing the proteins of the disclosure (i.e. the polypeptides making up the human CD200R-binding antibody or antigen-binding fragment thereof of the disclosure) is a mammalian cell such as a HEK293T or CHO-K1 cell. In a particular embodiment, the host cell is a mammalian cell, such as a HEK293T or CHO-K1 cell.

A variety of host-expression vector systems is suitable to express a CD200R-binding molecule as described herein. Different host cells have characteristic and specific mechanisms for the post-translational processing and modification of proteins and gene products. Appropriate cell lines or host systems is chosen to ensure the correct modification and processing of the protein of the disclosure. In some embodiments, eukaryotic host cells which possess the cellular machinery for proper processing of the primary transcript, glycosylation, and phosphorylation of the gene product is used. Such mammalian host cells include but are not limited to CHO, HEK, VERY, BHK, Hela, COS, MDCK, 293, 3T3, W138, BT483, Hs578T, HTB2, BT20 and T47D, NSO, CRL7030 and HsS78Bst cells.

Antibody Manufacturing

The CD200R antibody or fragment thereof disclosed herein can be produced as a recombinant antibody by cloning DNA encoding the subject antibody or peptide from hybridomas or B cells or any form of antibody and/or antibody fragment libraries, integrating the clone into a suitable vector, and transducing the vector into host cells (for example, P. J. Delves, Antibody Production: Essential Techniques, 1997 WILEY, P. Shepherd and C. Dean Monoclonal Antibodies, 2000 OXFORD UNIVERSITY PRESS, Vandamme A. M. et al., Eur. J. Biochem. 192:767-775 (1990)). Thus, in one aspect, provided herein is an isolated polynucleotide encoding an antibody or fragment thereof of the present disclosure.

Nucleotide sequences corresponding to various regions of L or H chains of an existing antibody can be readily obtained and sequenced using convention techniques including but not limited to hybridization, PCR, and DNA sequencing. Hybridoma cells that produce monoclonal antibodies serve as a preferred source of antibody nucleotide sequences. A vast number of hybridoma cells producing an array of monoclonal antibodies may be obtained from public or private repositories. The largest depository agent is American Type Culture Collection, which offers a diverse collection of well-characterized hybridoma cell lines. Alternatively, antibody nucleotides can be obtained from immunized or non-immunized rodents or humans, and from organs such as spleen and peripheral blood lymphocytes. Specific techniques applicable for extracting and synthesizing antibody nucleotides are described in Orlandi et al. (1989) Proc. Natl. Acad. Sci. U.S.A 86: 3833-3837, Larrick et al. 1989) biochem. Biophys. Res. Commun. 160: 1250-1255; Sastry et al. (1989) Proc. Natl. Acad. Sci., U.S.A. 86: 5728-5732; and U.S. Pat. No. 5,969,108.

The CD200R antibody nucleotide sequences may also be modified, for example, by substituting the coding sequence for human heavy and light chain constant regions in place of the homologous non-human sequences. In that manner, chimeric antibodies are prepared that retain the binding specificity of the original antibody.

Additionally, polynucleotides encoding the heavy and/or light chains of the CD200R antibody provided in this disclosure can be subjected to codon optimization to achieve optimized expression of a subject antibody or functional fragment thereof in a desired host cell. For example, in one method of codon optimization, a native codon is substituted by the most frequent codon from a reference set of genes, wherein the rate of codon translation for each amino acid is designed to be high. Additional exemplary methods for generating codon optimized polynucleotides for expression of a desired protein, which can be applied to the heavy and/or light chains of the CD200R antibody or a functional fragment thereof, are described in Kanaya et al., Gene, 238:143-155 (1999), Wang et al., Mol. Biol. Evol., 18(5):792-800 (2001), U.S. Pat. No. 5,795,737, U.S. Publication 2008/0076161 and WO 2008/000632.

Polynucleotides of the CD200R antibody of the present disclosure include those coding for functional equivalents and fragments thereof of the exemplified polypeptides. Functional equivalents may be polypeptides having conservative amino acid substitutions, analogs including fusions, and mutants.

Due to the degeneracy of the genetic code, there can be considerable variation in nucleotides of the L and H sequences, as well as the heterodimerization sequences suitable for construction of the polynucleotide and vectors of the present disclosure. These variation are encompassed by the present disclosure.

Where desired, the recombinant polynucleotides can comprise heterologous sequences that facilitate detection of the expression and purification of the gene product. Examples of such sequences include those encoding reporter proteins such as β-galactosidase, β-lactamase, chloramphenicol acetyltransferase (CAT), luciferase, green fluorescent protein (GFP) and their derivatives. Other heterologous sequences that facilitate purification may code for epitopes such as Myc, HA (derived from influenza virus hemagglutinin), His-6, FLAG, or the Fc portion of immunoglobulin, glutathione S-transferase (GST), and maltose-binding protein (MBP).

The polynucleotides can be conjugated to a variety of chemically functional moieties as described above. Commonly employed moieties include labels capable of producing a detectable signal, signal peptides, agents that enhance or reduce immunologic reactivity, agents that facilitate coupling to a solid support, vaccine carriers, bioresponse modifiers, paramagnetic labels and drugs.

The moieties can be covalently linked to a polynucleotide recombinantly or by other means known in the art.

The polynucleotides can comprise additional sequences, such as additional encoding sequences within the same transcription unit, controlling elements such as promoters, ribosome binding sites, and polyadenylation sites, additional transcription units under control of the same or a different promoter, sequences that permit cloning, expression, and transformation of a host cell, and any such construct as may be desirable in accordance with any of the various embodiments described herein.

The polynucleotides can be obtained using chemical synthesis, recombinant cloning methods, PCR, or any combination thereof. One of skill in the art can use the sequence data provided herein to obtain a desired polynucleotide by employing a DNA synthesizer or ordering from a commercial service.

Polynucleotides comprising a desired sequence can be inserted into a suitable vector which in turn can be introduced into a suitable host cell for replication, amplification and expression.

Accordingly, in one aspect, provided herein are a variety of vectors comprising one or more of the polynucleotides of the present disclosure. Also provided is a selectable library of expression vectors comprising at least one vector encoding the subject antibody.

In some aspects, provided herein is a polynucleotide sequence encoding at least a portion of the heavy chain or light chain of the antibody or fragment thereof disclosed herein. In some aspects, provided herein is a vector comprising the polynucleotide sequence disclosed herein.

Vectors of the present disclosure are generally categorized into cloning and expression vectors. Cloning vectors are useful for obtaining replicate copies of the polynucleotides they contain, or as a means of storing the polynucleotides in a depository for future recovery. Expression vectors (and host cells containing these expression vectors) can be used to obtain polypeptides produced from the polynucleotides they contain. Suitable cloning and expression vectors include any known in the art, e.g., those for use in bacterial, mammalian, yeast, insect and phage display expression systems.

Suitable cloning vectors can be constructed according to standard techniques, or selected from a large number of cloning vectors available in the art. While the cloning vector selected may vary according to the host cell intended to be used, useful cloning vectors will generally have the ability to self-replicate, may possess a single target for a particular restriction endonuclease, or may carry marker genes. Suitable examples include plasmids and bacterial viruses, e.g., pBR322, pMB9, ColEl, pCR1, RP4, pUC18, mpl8, mpl9, phage DNAs (including filamentous and non-filamentous phage DNAs), and shuttle vectors such as pSA3 and pAT28. These and other cloning vectors are available from commercial vendors such as Clontech, BiORad, Stratagene, and Invitrogen.

Expression vectors containing these nucleic acids are useful to obtain host vector systems to produce proteins and polypeptides. Typically, these expression vectors are replicable in the host organisms either as episomes or as an integral part of the chromosomal DNA. Suitable expression vectors include plasmids, viral vectors, including phagemids, adenoviruses, adeno-associated viruses, retroviruses, cosmids, etc. A number of expression vectors suitable for expression in eukaryotic cells including yeast, avian, and mammalian cells are available. One example of an expression vector is pcDNA3 (Invitrogen, San Diego, Calif.), in which transcription is driven by the cytomegalovirus (CMV) early promoter/enhancer. Two types of particularly useful expression vectors for expressing the subject antibody as described herein are the phage display vector and bacterial display vector.

The vectors of the present disclosure can comprise transcriptional or translational control sequences required for expressing the encoded antibody. Suitable transcription or translational control sequences include but are not limited to replication origin, promoter, enhancer, repressor binding regions, transcription initiation sites, ribosome binding sites, translation initiation sites, and termination sites for transcription and translation.

The expression vector can be transferred to a host cell and the transfected cells are then cultured to produce a subject antibody or functional fragment thereof. Thus, in one aspect, provided herein are host cells containing a polynucleotide encoding a subject antibody or functional fragment thereof operably linked to a heterologous promoter. The host cell can be co-transfected with two expression vectors, the first vector encoding a heavy chain derived polypeptide and the second vector encoding a light chain derived polypeptide. The two vectors can contain identical selectable markers which enable equal expression of heavy and light chain polypeptides. Alternatively, a single vector can be used which encodes, and is capable of expressing, both heavy and light chain polypeptides. In such situations, the light chain can be placed before the heavy chain to avoid an excess of toxic free heavy chain (Proudfoot, 1986, Nature 322:52; and Kohler, 1980, Proc. Natl. Acad. Sci. USA 77:2197-2199).

A variety of host-expression vector systems can be utilized to express the subject antibody or functional fragment thereof (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express a subject antibody molecule in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In some embodiments, antibodies or fragments thereof are produced in CHO cells.

For bacterial systems, a number of expression vectors may be advantageously selected depending upon the use intended for the antibody molecule being expressed. For example, when a large quantity of such an antibody or fragment thereof is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruther et al., 1983, EMBO 12:1791), in which the antibody coding sequence can be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) can be used as a vector to express foreign genes. The virus grows in Spodoptera frugiperda cells. The antibody or functional fragment coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region El or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals can also be used for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bittner et al., 1987, Methods in Enzymol. 153:51-544).

For plant cells, a variety of vector delivery techniques is available in the art. The host cells may be in the form of whole plants, isolated cells or protoplasts. Illustrative procedures for introducing vectors into plant cells include Agrobacterium-mediated plant transformation, protoplast transformation, gene transfer into pollen, injection into reproductive organs and injection into immature embryos. As is evident to one skilled in the art, each of these methods has distinct advantages and disadvantages. Thus, one particular method of introducing vectors into a particular plant species may not necessarily be the most effective for another plant species.

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the antibody or functional fragment. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERY, BHK, Hela, COS, MDCK, 293, 3T3, W138, BT483, Hs578T, HTB2, BT20 and T47D, NSO (a murine myeloma cell line that does not endogenously produce any immunoglobulin chains), CRL7030 and HsS78Bst cells.

For long-term, high-yield production of recombinant proteins, stable expression is preferred. For example, cell lines which stably express an antibody or functional fragment thereof can be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express the antibody molecule.

In some embodiments, a number of selection systems are used, including but not limited to, systems using the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes in tk-, hgprt- or aprt-cells, respectively. Also, antimetabolite resistance can be used as the basis of selection for the following genes: dhfr, which confers resistance to methotrexate (Wigler et al., 1980, Proc. Natl. Acad. Sci. USA. 77(6):3567-70; O'Hare et al., 1981, Proc. Natl. Acad. Sci. USA 78:1527); glutamine synthetase (GS), which is an enzyme responsible for the biosynthesis of glutamine using glutamate and ammonia (Bebbington et al., 1992, Biuotechnology 10:169); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-215); and hygro, which confers resistance to hygromycin (Santerre et al., 1984, Gene 30:147). Recombinant DNA technology methods can be applied to select the desired recombinant clone, and such methods are described, for example, in Ausubel et al. (eds.), Current Protocols in Molecular Biology, John Wiley & Sons, N Y (1993); Kriegler, Gene Transfer and Expression, A Laboratory Manual, Stockton Press, N Y (1990); and in Chapters 12 and 13, Dracopoli et al. (eds.), Current Protocols in Human Genetics, John Wiley & Sons, N Y (1994); Colberre-Garapin et al., 1981, J. Mol. Biol. 150:1, which are incorporated by reference herein in their entireties. The expression levels of an antibody molecule can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing an antibody or functional fragment thereof is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

Once an antibody molecule has been produced by recombinant expression, it can be purified by any suitable method for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the subject antibodies or functional fragments thereof can be fused to heterologous polypeptide sequences provided herein or otherwise known in the art to facilitate purification. For example, a subject antibody or functional fragment thereof can be purified through recombinantly adding a poly-histidine tag (His-tag), FLAG-tag, hemagglutinin tag (HA-tag) or myc-tag among others that are commercially available and utilizing suitable purification methods.

Pharmaceutical Composition

In another aspect, provided herein are pharmaceutical compositions comprising the anti-CD200R antibody or a functional fragment thereof disclosed herein, and a pharmaceutically acceptable carrier or excipient. In another aspect, provided herein are pharmaceutical compositions comprising an immunoconjugate comprising the antibody disclosed herein, and a pharmaceutically acceptable carrier or excipient. The pharmaceutically acceptable carrier or excipient can include, but not limited to, inert solid diluents and fillers, diluents, sterile aqueous solution and various organic solvents, permeation enhancers, solubilizers and adjuvants. These compositions can be formulated according to known methods for preparing pharmaceutically useful compositions. Formulations are described in a number of sources which are well known and readily available to those skilled in the art. For example, Remington's Pharmaceutical Science (Martin E. W., Easton Pennsylvania, Mack Publishing Company, 19^{th} ed., 1995) describes formulations which can be used in connection with the antibody or antigen-binding fragment thereof or immunoconjugate of the present disclosure.

The pharmaceutical composition disclosed herein can, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. Suitable examples of sustained release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and Q ethyl-L-glutamate, non-degradable ethylene-vinyl acetate, degradable lactic acid-glycolic acid copolymers such as those used in LUPRON DEPOT™ (injectable microspheres composed of lactic acid-glycolic acid copolymer and leuprolide acetate), and poly-D-(−)-3-hydroxybutyric acid. Some sustained release formulations enable release of molecules over a few weeks to a few months, or even up to a few years. In some embodiments, the subject pharmaceutical composition release the subject antibody as described herein for at least a few weeks, such as for at least 1 week, 2 weeks, 3 weeks or 4 weeks. In further embodiments, the subject pharmaceutical composition release the subject antibody as described herein over a few months, such as for at least 1 month, 2 months, 3 months, 4 months, 5 months, or 6 months.

The pharmaceutical composition disclosed herein can be in unit dosage forms suitable for single administration of precise dosages. The pharmaceutical composition can further comprise an antibody or a functional fragment thereof as an active ingredient and may include a conventional pharmaceutical carrier or excipient. Further, it may include other medicinal or pharmaceutical agents, carriers, adjuvants, etc.

Exemplary parenteral administration forms include solutions or suspensions of active polypeptide and/or PEG-modified polypeptide in sterile aqueous solutions, for example, aqueous propylene glycol or dextrose solutions. Such dosage forms can be suitably buffered with salts such as histidine and/or phosphate, if desired.

Formulations suitable for administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents.

The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc.

In some embodiments, the disclosure provides a pharmaceutical composition for injection containing a subject antibody or a functional fragment thereof and a pharmaceutical excipient suitable for injection. Example components and amounts of agents in such compositions are as described herein.

The forms in which the compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline can be used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions can be prepared by incorporating an antibody of the present disclosure or functional fragment thereof in the desired amount in the appropriate solvent with various other ingredients as enumerated above, followed by filtered sterilization. Generally, dispersions are prepared by incorporating the various sterilized active ingredients into a sterile vehicle which contains the basic dispersion medium and other ingredients. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

In some embodiments, the disclosure provides a pharmaceutical composition for oral administration containing an antibody of the present disclosure or a functional fragment thereof, and a pharmaceutical excipient suitable for oral administration.

In some embodiments, a solid pharmaceutical composition for oral administration is provided herein containing: (i) an effective amount of an antibody of the present disclosure or a functional fragment thereof; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition is a liquid pharmaceutical composition suitable for oral consumption. Pharmaceutical compositions suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, and typically include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation.

This disclosure further encompasses anhydrous pharmaceutical compositions and dosage forms comprising an active ingredient, since water can facilitate the degradation of some polypeptides. For example, water may be added (e.g., 5%) in the pharmaceutical arts as a means of simulating long-term storage in order to determine characteristics such as shelf-life or the stability of formulations over time. Anhydrous pharmaceutical compositions and dosage forms can be prepared using anhydrous or low moisture containing ingredients and low moisture or low humidity conditions. Pharmaceutical compositions and dosage forms which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An antibody of the present disclosure can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants can be used in the compositions to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof, lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof, polyoxyethylated vitamins and derivatives thereof, polyoxyethylene-polyoxypropylene block copolymers; and mixtures thereof, polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In some cases the composition includes a solubilizer to ensure good solubilization and/or dissolution of the compound and to minimize precipitation of the compound. This can be especially advantageous for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

Kits

Disclosed herein are kits comprising any of the antibody or antigen-binding fragments thereof provided herein. In another aspect of the disclosure, provided are kits comprising the unit doses containing any of the antibody or antigen-binding fragments thereof provided herein and instructions for use. The kit can further comprise one or more unit doses containing one or more additional reagents, such as an immunosuppressive reagent as described above, or one or more additional antibodies as described herein (e.g., a human antibody having a complementary activity which binds to an epitope in the antigen distinct from a first human antibody). Kits typically include a label indicating the intended use of the contents of the kit. The term label includes any writing, or recorded material supplied on or with the kit, or which otherwise accompanies the kit.

Disclosed herein is a kit comprising an antibody or antigen-binding fragment thereof that comprises a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 70, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some embodiments, the HCVR comprises an amino acid sequence having at least 90%, 95%, 96%, 97%, 98%, 99%, or 100% sequence identity to the amino acid sequence as set forth in SEQ ID NO: 72. In some embodiments, the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 72, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications.

Disclosed herein is a kit comprising an antibody or antigen-binding fragment thereof that comprises a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 5, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some embodiments, the HCVR comprises an amino acid sequence having at least 90%, 95%, 96%, 97%, 98%, 99%, or 100% sequence identity to the amino acid sequence as set forth in SEQ ID NO: 71. In some embodiments, the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 71, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications.

Disclosed herein is a kit comprising an antibody or antigen-binding fragment thereof that comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 4, 5, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 7, 8, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 1. In some embodiments, the LCVR comprises an amino acid sequence having at least 90%, 95%, 96%, 97%, 98%, 99%, or 100% sequence identity to the amino acid sequence as set forth in SEQ ID NO: 2. In some embodiments, the HCVR comprises an amino acid sequence as set forth in SEQ ID NO: 1, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 2, with from 0 to 10 amino acid modifications, such as 1, 2, 3, 4, 5, 6, 7, 8, 9 or 10 amino acid modifications.

Disclosed herein is a kit comprising an antibody or antigen-binding fragment thereof that comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 11, 12, 13, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 14, 15, 16, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 9. In some embodiments, the LCVR comprises an amino acid sequence having at least 90%, 95%, 96%, 97%, 98%, 99%, or 100% sequence identity to the amino acid sequence as set forth in SEQ ID NO: 10. In some embodiments, the HCVR comprises an amino acid sequence as set forth in SEQ ID NO: 9, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 10, with from 0 to 10 amino acid modifications, such as 1, 2, 3, 4, 5, 6, 7, 8, 9 or 10 amino acid modifications.

Disclosed herein is a kit comprising an antibody or antigen-binding fragment thereof that comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 19, 20, or 21, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 22, 23, or 24, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 17. In some embodiments, the LCVR comprises an amino acid sequence having at least 90%, 95%, 96%, 97%, 98%, 99%, or 100% sequence identity to the amino acid sequence as set forth in SEQ ID NO: 18. In some embodiments, the HCVR comprises an amino acid sequence as set forth in SEQ ID NO: 17, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 18, with from 0 to 10 amino acid modifications, such as 1, 2, 3, 4, 5, 6, 7, 8, 9 or 10 amino acid modifications.

Disclosed herein is a kit comprising an antibody or antigen-binding fragment thereof that comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 27, 28, or 29, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 30, 31, or 32, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 25. In some embodiments, the LCVR comprises an amino acid sequence having at least 90%, 95%, 96%, 97%, 98%, 99%, or 100% sequence identity to the amino acid sequence as set forth in SEQ ID NO: 26. In some embodiments, the HCVR comprises an amino acid sequence as set forth in SEQ ID NO: 25, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 26, with from 0 to 10 amino acid modifications, such as 1, 2, 3, 4, 5, 6, 7, 8, 9 or 10 amino acid modifications.

Disclosed herein is a kit comprising an antibody or antigen-binding fragment thereof that comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 35, 36, or 37, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 38, 39, or 40, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 33. In some embodiments, the LCVR comprises an amino acid sequence having at least 90%, 95%, 96%, 97%, 98%, 99%, or 100% sequence identity to the amino acid sequence as set forth in SEQ ID NO: 34. In some embodiments, the HCVR comprises an amino acid sequence as set forth in SEQ ID NO: 33, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 34, with from 0 to 10 amino acid modifications, such as 1, 2, 3, 4, 5, 6, 7, 8, 9 or 10 amino acid modifications.

A kit of the present disclosure may also include diagnostic agents and/or other therapeutic agents. In some cases a kit includes an antibody of the present disclosure and a diagnostic agent that may be used in a diagnostic method for diagnosing the state or existence of a disease, condition or disorder in a subject as described herein.

Methods of Treatment

In another aspect, provided herein are methods of using the antibody or a functional fragment thereof disclosed herein to suppress an immune cell expressing CD200R. In some cases, the method of suppressing the immune cell expressing CD200R using the antibody disclosed herein is in vitro, ex vivo, or in vivo. In some cases, the method of suppressing an immune cell that expresses CD200R comprises contacting the immune cell with the antibody or a functional fragment thereof disclosed herein. In some cases, the immune cell is a T cell, B cell, a macrophage, or any other immune cell. In some cases, the immune cell is an effector T cell. In some cases, the immune cell is an antigen-specific T cell. In some cases, the method disclosed herein is applicable to treat a subject in need thereof. In some cases, the method comprises administering the antibody or fragment thereof disclosed herein to a subject in need thereof. In some cases, the method comprises suppressing an immune cell in vitro and transferring the immune cell to a subject in need thereof. In some cases, the subject is a mammal. In some cases, the subject is a human subject.

In another aspect, provided herein are methods of using the antibody or antigen-binding fragment thereof, a functional fragment thereof, an immunoconjugate, or a pharmaceutical composition disclosed herein to downregulate an immune response in a subject. In some cases, the method disclosed herein is applicable to treat a subject in need thereof. In some cases, the method comprises administering a functional fragment thereof, an immunoconjugate, or a pharmaceutical composition disclosed herein to a subject in need thereof. In some cases, the method comprises downregulating an immune response in vitro and transferring the immune cell to a subject in need thereof. In some cases, the subject is a mammal. In some cases, the subject is a human subject.

In another aspect, the antibodies of the disclosure can be used as a targeting agent for delivery of another therapeutic or a cytotoxic agent (e.g., a toxin) to a cell expressing CD200R. The method includes administering an anti-CD200R antibody coupled to a therapeutic or a cytotoxic agent (e.g., in the form of an immunoconjugate of the present disclosure) or under conditions that allow binding of the antibody to CD200R expressed on the cell surface.

In another aspect, provided herein are methods of using the antibody, a functional fragment thereof, an immunoconjugate, or a pharmaceutical composition disclosed herein to treat, prevent, alleviate, or reduce the severity of diseases or conditions in a subject in need thereof. In some cases, the diseases or conditions the subject method is applicable to are associated with CD200R signaling. In some cases, the diseases or conditions are inflammatory disorder, autoimmune disorders, and/or associated with excess or undesirable immune response. In some embodiments, administering the antibody or antigen-binding fragment thereof comprises parenteral, intravenous, oral, subcutaneous, intra-arterial, intracranial, intrathecal, intraperitoneal, intratumoral, topical, intranasal or intramuscular administration. In some embodiments, administering the antibody or antigen-binding fragment thereof comprises intravenous, subcutaneous, or intramuscular administration.

In some embodiments, the present disclosure provides a method of treating, preventing, alleviating, or reducing the severity of a disease or condition in a mammal, e.g., a human, in need thereof, comprising administering to the mammal a therapeutically effective amount of an antibody of the present disclosure. In some embodiments, the present disclosure provides a method of treating, preventing, alleviating, or reducing the severity of disease or condition in a mammal, e.g., a human, in need thereof, comprising administering to the mammal a therapeutically effective amount of an immunoconjugate or a pharmaceutical composition comprising the antibody of the present disclosure. In some cases, the disease or condition is selected from adult-onset Still's disease, alcoholic hepatitis, alcoholic steatohepatitis, alcoholic liver disease, asthma, including allergen-induced asthma, bullous pemphigoid (BP) asthma, non-allergen induced asthma, allergies and allergic conditions such as allergic bronchopulmonary aspergillosis, allergic conjunctivitis, allergic encephalomyelitis, and allergic neuritis, food allergies, allograft rejection, alcoholic steatohepatitis (ASH), ANCA vasculitis, anti-glomerular basement membrane disease (Anti-GBM), antiphospholipid syndrome, aphthous stomatitis, appendicitis, arthritis, autoimmune diseases, atrophic thyroiditis, autoimmune hemolytic anemia (immune pancytopenia, paroxysmal nocturnal hemoglobinuria), autoimmune polyendocrinopathies, autoimmune thrombocytopenia (idiopathic thrombocytopenic purpura, immune-mediated thrombocytopenia), autoimmune hepatitis, pernicious anemia (Addison's disease), and autoimmune thyroid disorders, autoinflammatory diseases, autosomal dominant polyscystic kidney disease (ADPKD), ankylosing spondylitis (AS), acute respiratory distress syndrome (ARDS), Behcet's disease or syndrome, bee sting-induced inflammation, Blau syndrome, bursitis, Barrett's esophagus, bleomycin induced pulmonary fibrosis, bronchiolitis obliterans, cardiac hypertrophy, gluten-sensitive enteropathy (Celiac disease), chemical irritant-induced inflammation, chorioretinitis, chronic atypical neutrophilic dermatosis with lipodystrophy and elevated temperature (CANDLE) syndrome, chronic obstructive pulmonary disease (COPD), chronic pancreatitis, chronic prostatitis, chronic recurrent multifocal osteomyelitis, cicatricial alopecia, colitis, complex regional pain syndrome, chronic intrahepatic or extrahepatic cholestatic disease, conjunctivitis, connective tissue disease, Connective tissue disease-associated interstitial lung disease (CTD-ILD), corneal ulcer, cryopyrin-associated periodic syndromes, cutaneous lupus erythematosus (CLE), cystic fibrosis, deficiency of the interleukin-1 receptor antagonist (DIRA), deficiency of IL36R antagonist (DITRA), dermatitis, diabetic kidney disease (DKD) (diabetic nephropathy), diverticulitis, discoid lupus erythematosus, drug induced delayed type cutaneous allergic reactions, encephalitis, esophagitis, eosinophilic gastrointestinal disorders (EGIDs), such as eosinophilic esophagitis (EoE), eosinophilic gastroenteritis, eosinophilic colitis, familial cold urticarial, familial Mediterranean fever, fistulizing Crohn's disease, giant cell arteritis, glomerulonephritis,g out, gouty arthritis, graft-versus-host disease (GVHD), granulomatous hepatitis, Guillain-Barre syndrome (GBS), Graves' disease, Hashimoto's thyroiditis, Henoch-Schonlein purpura, hidradenitis suppurativa (HS), hyaline membrane disease, hyperactive inflammatory response, hypereosinophilic syndrome (HES), hyperimmunoglobulinemia D with recurrent fever (HIDS), hypersensitivity pneumonitis (HP), immunoglobulin (IgA) nephropathies, IgG4-related disease, immune complex nephritis, immune thrombocytopenic purpura (ITP), inflammation, inflammation of the CNS, inflammatory bowel disease (IBD), inflammatory disease of the respiratory tract (upper or lower) such as inflammatory lung disease, bronchitis, sinusitis, inflammatory ischemic event such as stroke or cardiac arrest, inflammatory liver disease, inflammatory myopathy, inflammatory neuropathy, inflammatory pain, insect bite-induced inflammation, interstitial cystitis, iritis, irritant-induced inflammation, juvenile arthritis, juvenile rheumatoid arthritis, keratitis, kidney transplant rejection, kidney disease, kidney fibrosis, kidney insufficiency, leukocyte adhesion deficiency, Loeffler's syndrome, lupus, lupus nephritis (LN), liver fibrosis, liver steatosis, liver ischemia, lipid and lipoprotein disorders, mast cell activation syndrome, mastocytosis, meningitis, microscopic colitis, mixed connective tissue disease, morphea or morphea variants, Muckle-Wells syndrome (urticaria deafness amyloidosis), mucositis, myelitis, myocarditis, myositis, necrotizing enterocolitis, neonatal onset multisystem inflammatory disease (NOMID), nasal polyps, neovascular glaucoma, neuritis, non-alcoholic fatty liver disease (NAFLD), non-alcoholic steatohepatitis (NASH), non-radiographic axial spondyloarthritis (nr-AxSpA), non-cystic fibrosis bronchiectasis (non-CFB), obstructive or chronic inflammatory disorders of the liver, ocular allergy, optic neuritis, organ transplant rejection, osteoarthritis (OA), otitis, pancreatitis, pancolitis, pelvic inflammatory disease, pemphigus vulgaris (PV), bullous pemphigoid (BP), pericarditis, periodontitis, PFAPA (periodic fever, aphthous stomatitis, pharyngitis, adenitis), plant irritant-induced inflammation, pneumocystis infection, pneumonia, pneumonitis, poison ivy/urushiol oil-induced inflammation, polyarteritis nodosa, polychondritis, polycystic kidney disease (PCKD), polymyalgia rheumatic, polymyositis, pouchitis, proctitis, proctosignmoiditis, psoriatic arthritis (PsA), pulmonary arterial hypertension (PAH), pulmonary fibrosis, pyogenic sterile arthritis, pruritus, reperfusion injury and transplant rejection, primary biliary cirrhosis (PBC), primary sclerosing cholangitis (PSC), Raynaud's syndrome, Reiter's disease, reactive arthritis, renal graft rejection, reperfusion injury, rheumatic carditis, rheumatic diseases, rheumatic fever, rheumatoid arthritis (RA), rhinitis, rhinitis psoriasis, sarcoidosis, Schnitzler syndrome, scleritis, sclerosis, such as systemic sclerosis (SSc), seborrhea, sepsis, septic shock, Sjogren's syndrome, inflammatory skin diseases or conditions, such as acne, alopecia areata, atopic dermatitis, rosacea, eczema, dermatitis, dermatitis endotoxemia, dermatomyositis, stasis dermatitis, Stevens-Johnson syndrome (SJS), skin irritation, skin rash, skin sensitization (contact dermatitis or allergic contact dermatitis), scleroderma, psoriasis, psoriasis vulgaris, psoriatic arthritis, , spinal stenosis, spondyloarthropathies, synovial inflammation, systemic inflammatory response syndrome (SIRS), systemic lupus erythematosus (SLE), systemic mast cell disease (SMCD), systemic vasculitis, systemic-onset juvenile idiopathic arthritis, temporal arteritis, tendinitis, tenosynovitis, thyroditis, transplantation rejection, tubulointerstitial nephritis, tubular disfunction, Takayasu arteritis, toxic epidermal necrolysis, urticaria, uterine fibroids, uveitis, uveoretinitis, vasculitis, vasculitis (NHLBI), vitiligo, Wegener's granulomatosis, acne, acid-induced lung injury, Addison's disease, adrenal hyperplasia, adrenocortical insufficiency, age-related macular degeneration, aging, alcoholic liver disease, Alzheimer's disease, angina pectoris, angiofibroma, anhidrotic ectodermal dysplasia, ascites, aspergillosis, atherosclerosis, atherosclerotic plaques, amyloidosis, amyotrophic lateral sclerosis (ALS), angioedema, acute myocardial infarction; antigen-antibody complex mediated diseases, alpha-1-antitrypsin deficiency; back pain, Bacillus anthracis infection, Bell's palsy, berylliosis, bone pain, burns, bullous pemphigoid, cancer, carpal tunnel syndrome, Castleman's disease, catabolic disorders, cataracts, cerebral aneurysm, complications of organ transplantation, corneal graft neovascularization, cryptococcosis, a non-malignant hyperproliferative disorder; a malignant hyperproliferative disorder; hepatocellular carcinoma; colon adenoma; polyposis; colon adenocarcinoma; breast cancer; pancreatic adenocarcinoma, chronic heart failure, chronic lung disease of prematurity, cardiometabolic syndrome, cardiovascular disease, cutaneous T cell lymphoma, diabetic macular edema, dyslipidemia; endometriosis, endotoxemia, eosinophilic GI disease (EGID), eosinophilic esophagitis (EoE), eosinophilic pneumonias, epicondylitis, epidermolysis bullosa, erythema multiforme, erythroblastopenia, familial amyloidotic polyneuropathy, fetal growth retardation, fibromyalgia, glaucoma, glioblastoma, glomerular disease, gut diseases, growth plate injuries, hair loss, herpes zoster and simplex, hypoplastic and other anemias, head injury, hepatitis A, B, C, D, and E, herpes; headache, hearing loss, heart disease, hemangioma, hemophilic joints, hereditary periodic fever syndrome, heritable disorders of connective tissue, Hodgkin's disease, Huntington's disease, hyperammonemia, hypercalcemia, hypercholesterolemia, hemolytic anemia, hepatitis, hip replacement, hypertropic bone formation, hypersensitivity pneumonia, hereditary fructose intolerance, hypertension, hyperuricemia, idiopathic demyelinating polyneuropathy, infectious diseases including viral diseases such as AIDS (HIV infection), ichthyosis, incontinentia pigmenti (TP, Bloch-Siemens syndrome), idiopathic thrombocytopenic purpura, infectious mononucleosis, ischemia/reperfusion, insulin resistance, joint replacement, kidney injury caused by parasitic infections, leptospirosis, lichen sclerosus (LS), lichen planus, Lambert-Eaton myasthenic syndrome, Lyme disease, liver failure, including acute liver failure, muscle wasting, muscular dystrophy, Marfan syndrome (MFS), meningioma, mesothelioma, multiple organ injury syndrome, myasthenia gravis (MG), myelodysplastic syndrome, metabolic syndrome, multiple sclerosis, nephrotic syndrome, neuropathological diseases, nuclear factor-kappa B essential modulator (NEMO) deficiency syndrome, obesity, Osler-Weber syndrome, osteogenesis imperfecta, osteonecrosis, osteoporosis, pachyonychia congenita, Paget's disease, Paget's disease of bone, Parkinson's disease, periodic fever, pertussis, primary pulmonary hypertension, pyoderma gangrenosum, pyogenic granuloma retrolental fibroplasias, peritoneal endometriosis, Prurigo nodularis, psychosocial stress diseases, pulmonary disease, pulmonary hypertension, respiratory distress syndrome, renal disease, retinal disease, retrolental fibroplasia, renal transplant rejection, renal protection against drugs inducing Fanconi's syndrome, respiratory tract illness caused by respiratory syncytial virus, rhinosinusitis; radiation induced fibrosis, sarcoidosis, severe pain, sleep apnea, scoliosis, sickle cell anemia, sports injuries, sprains and strains, sunburn, spinal cord injury, Sezary syndrome, silica-induced disease (Silicosis), subarachnoid hemorrhage, tuberculosis, tumor necrosis factor (TNF) receptor associated periodic syndrome (TRAPS), thrombosis; traumatic brain injury, tissue transplant, complications from type 1 or type 2 diabetes, toxoplasmosis, thrombocytopenia, trachoma, vascular restenosis, ventilator induced lung injury, Whipple's disease, and 2,8-dihydroxyadenine nephropathy. In some embodiments, the disease or condition is selected from rheumatological disease or condition, gastrointestinal disease or condition, pulmonary disease or condition, hepatological disease or condition, nephrological disease or condition, or dermatological disease or condition. In some embodiments, the rheumatological condition or disease is selected from rheumatoid arthritis (RA), systemic lupus erythematosus (SLE), lupus nephritis (LN), osteoarthritis (OA), Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, Takayasu arteritis, cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the rheumatological condition or disease is selected from rheumatoid arthritis (RA), systemic lupus erythematosus (SLE), lupus nephritis (LN), and osteoarthritis (OA). In some embodiments, the rheumatological condition or disease is selected Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, and Takayasu arteritis. In some embodiments, the rheumatological condition or disease is selected cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the gastrointestinal disease or condition is selected from ulcerative colitis (UC), Crohn's disease (CD), eosinophilic gastrointestinal disorders (EGIDs), microscopic colitis, ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the gastrointestinal disease or condition is selected from ulcerative colitis (UC) and Crohn's disease (CD). In some embodiments, the gastrointestinal disease or condition is selected from eosinophilic gastrointestinal disorders (EGIDs) and microscopic colitis. In some embodiments, the EGID is selected from eosinophilic esophagitis (EoE), eosinophilic gastroenteritis, and eosinophilic colitis. In some embodiments, the microscopic colitis is selected from collagenous colitis and lymphocytic colitis. In some embodiments, the gastrointestinal disease is selected from ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the pulmonary disease or condition is selected from idiopathic pulmonary fibrosis (IPF), interstitial lung disease (ILD), acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the pulmonary disease or condition is selected from idiopathic pulmonary fibrosis (IPF) and interstitial lung disease (ILD). In some embodiments, the pulmonary disease or condition is selected from acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the hepatological disease or condition is selected from non-alcoholic steatohepatitis (NASH), primary sclerosing cholangitis (PSC), primary biliary cirrhosis (PBC), autoimmune hepatitis, alcoholic steatohepatitis (ASH), alcoholic hepatitis, chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the hepatological disease or condition is non-alcoholic steatohepatitis (NASH). In some embodiments, the hepatological disease or condition is selected from primary sclerosing cholangitis (PSC), primary biliary cirrhosis (PBC), autoimmune hepatitis, alcoholic steatohepatitis (ASH), and alcoholic hepatitis. In some embodiments, the hepatological disease or condition is selected from chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the nephrological disease or condition is selected from diabetic kidney disease (DKD) (diabetic nephropathy), chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the nephrological disease or condition is diabetic kidney disease (DKD) (diabetic nephropathy). In some embodiments, the nephrological disease or condition is selected from chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the dermatological disease or condition is selected from atopic dermatitis (AD), contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea. In some embodiments, the dermatological disease or condition is atopic dermatitis (AD). In some embodiments, the dermatological disease or condition is selected from contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea. In some cases, the disease or condition comprises rheumatoid arthritis. In some cases, the disease or condition comprises multiple sclerosis. In some embodiments, administering the antibody or antigen-binding fragment thereof comprises parenteral, intravenous, oral, subcutaneous, intra-arterial, intracranial, intrathecal, intraperitoneal, intratumoral, topical, intranasal or intramuscular administration. In some embodiments, administering the antibody or antigen-binding fragment thereof comprises intravenous, subcutaneous, or intramuscular administration. In some embodiments, the antibody or antigen-binding fragment thereof comprises a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 70, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some embodiments, the HCVR comprises an amino acid sequence having at least 90%, 95%, 96%, 97%, 98%, 99%, or 100% sequence identity to the amino acid sequence as set forth in SEQ ID NO: 72. In some embodiments, the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 72, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications. In some embodiments, the antibody or antigen-binding fragment thereof comprises a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 5, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some embodiments, the HCVR comprises an amino acid sequence having at least 90%, 95%, 96%, 97%, 98%, 99%, or 100% sequence identity to the amino acid sequence as set forth in SEQ ID NO: 71. In some embodiments, the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 71, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications. In some embodiments, the antibody or antigen-binding fragment thereof comprises a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 92, 41, and 69, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 87, and 91, respectively, each with 0 to 3 amino acid modifications, such as 0, 1, 2, or 3 modifications. In some embodiments, X at position 1 of SEQ ID NO: 69 is M. In some embodiments, X at position 1 of SEQ ID NO: 69 is G. In some embodiments, X at position 1 of SEQ ID NO: 87 is G. In some embodiments, X at position 1 of SEQ ID NO: 87 is L. In some embodiments, X at position 2 of SEQ ID NO: 87 is A. In some embodiments, X at position 2 of SEQ ID NO: 87 is G. In some embodiments, X at position 3 of SEQ ID NO: 87 is S. In some embodiments, X at position 3 of SEQ ID NO: 87 is V. In some embodiments, X at position 7 of SEQ ID NO: 87 is D. In some embodiments, X at position 7 of SEQ ID NO: 87 is S. In some embodiments, X at position 7 of SEQ ID NO: 87 is T. In some embodiments, X at position 8 of SEQ ID NO: 91 is W. In some embodiments, X at position 8 of SEQ ID NO: 91 is F. In some embodiments, X at position 3 of SEQ ID NO: 92 is W. In some embodiments, X at position 3 of SEQ ID NO: 92 is F. In some embodiments, the HCVR comprises an amino acid sequence having at least 90%, 95%, 96%, 97%, 98%, 99%, or 100% sequence identity to the amino acid sequence as set forth in SEQ ID NO: 93. In some embodiments, the LCVR comprises an amino acid sequence having at least 90%, 95%, 96%, 97%, 98%, 99%, or 100% sequence identity to the amino acid sequence as set forth in SEQ ID NO: 94. In some embodiments, the HCVR comprises an amino acid sequence as set forth in SEQ ID NO: 93, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 94, with from 0 to 10 amino acid modifications, such as 1, 2, 3, 4, 5, 6, 7, 8, 9 or 10 amino acid modifications. In some embodiments, X at position 1 of SEQ ID NO: 93 is D. In some embodiments, X at position 1 of SEQ ID NO: 93 is E. In some embodiments, X at position 33 of SEQ ID NO: 93 is W. In some embodiments, X at position 33 of SEQ ID NO: 93 is F. In some embodiments, X at position 99 of SEQ ID NO: 93 is M. In some embodiments, X at position 99 of SEQ ID NO: 93 is G. In some embodiments, X at position 50 of SEQ ID NO: 94 is G. In some embodiments, X at position 50 of SEQ ID NO: 94 is L. In some embodiments, X at position 51 of SEQ ID NO: 94 is A. In some embodiments, X at position 51 of SEQ ID NO: 94 is G. In some embodiments, X at position 52 of SEQ ID NO: 94 is S. In some embodiments, X at position 52 of SEQ ID NO: 94 is V. In some embodiments, X at position 56 of SEQ ID NO: 94 is D. In some embodiments, X at position 56 of SEQ ID NO: 94 is S. In some embodiments, X at position 56 of SEQ ID NO: 94 is T. In some embodiments, X at position 96 of SEQ ID NO: 94 is W. In some embodiments, X at position 96 of SEQ ID NO: 94 is F. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 4, 5, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 7, 8, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 1. In some embodiments, the LCVR comprises an amino acid sequence having at least 90%, 95%, 96%, 97%, 98%, 99%, or 100% sequence identity to the amino acid sequence as set forth in SEQ ID NO: 2. In some embodiments, the HCVR comprises an amino acid sequence as set forth in SEQ ID NO: 1, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 2, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 11, 12, 13, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 14, 15, 16, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 9. In some embodiments, the LCVR comprises an amino acid sequence having at least 90%, 95%, 96%, 97%, 98%, 99%, or 100% sequence identity to the amino acid sequence as set forth in SEQ ID NO: 10. In some embodiments, the HCVR comprises an amino acid sequence as set forth in SEQ ID NO: 9, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 10, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 19, 20, or 21, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 22, 23, or 24, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 17. In some embodiments, the LCVR comprises an amino acid sequence having at least 90%, 95%, 96%, 97%, 98%, 99%, or 100% sequence identity to the amino acid sequence as set forth in SEQ ID NO: 18. In some embodiments, the HCVR comprises an amino acid sequence as set forth in SEQ ID NO: 17, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 18, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 27, 28, or 29, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 30, 31, or 32, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 25. In some embodiments, the LCVR comprises an amino acid sequence having at least 90%, 95%, 96%, 97%, 98%, 99%, or 100% sequence identity to the amino acid sequence as set forth in SEQ ID NO: 26. In some embodiments, the HCVR comprises an amino acid sequence as set forth in SEQ ID NO: 25, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 26, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 35, 36, or 37, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 38, 39, or 40, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 33. In some embodiments, the LCVR comprises an amino acid sequence having at least 90%, 95%, 96%, 97%, 98%, 99%, or 100% sequence identity to the amino acid sequence as set forth in SEQ ID NO: 34. In some embodiments, the HCVR comprises an amino acid sequence as set forth in SEQ ID NO: 33, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 34, with from 0 to 10 amino acid modifications, such as 1, 2, 3, 4, 5, 6, 7, 8, 9 or 10 amino acid modifications. In some embodiments, the LCVR is linked to a light chain constant region comprising an amino acid sequence having at least 90%, 95%, 96%, 97%, 98%, 99%, or 100% sequence identity to the amino acid sequence as set forth in SEQ ID NO: 62. In some embodiments, the light chain variable region is linked to a light chain constant region comprising an amino acid sequence as set forth in SEQ ID NO: 62, with from 0 to 10 amino acid modifications, such as 1, 2, 3, 4, 5, 6, 7, 8, 9 or 10 amino acid modifications. In some embodiments, the light chain variable region is linked to a light chain constant region comprising an amino acid sequence having at least 90%, 95%, 96%, 97%, 98%, 99%, or 100% sequence identity to the amino acid sequence as set forth in SEQ ID NO: 64. In some embodiments, the light chain variable region is linked to a light chain constant region comprising an amino acid sequence as set forth in SEQ ID NO: 64, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 61. In some embodiments, the HCVR is linked to a heavy chain constant region comprising an amino acid sequence as set forth in SEQ ID NO: 61, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 63. In some embodiments, the HCVR is linked to a heavy chain constant region comprising an amino acid sequence as set forth in SEQ ID NO: 63, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 75. In some embodiments, the HCVR is linked to a heavy chain constant region comprising an amino acid sequence as set forth in SEQ ID NO: 75, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 76. In some embodiments, the HCVR is linked to a heavy chain constant region comprising an amino acid sequence as set forth in SEQ ID NO: 76, with from 0 to 10 amino acid modifications, such as 1, 2, 3, 4, 5, 6, 7, 8, 9 or 10 amino acid modifications. In some embodiments, the HCVR is linked to a heavy chain constant region, wherein the heavy chain constant region comprises an Fc region that comprises one or more of the following amino acids: alanine (A) at position 234, alanine (A) at position 235, aspartic acid (D) at position 236, aspartic acid (D) at position 237, aspartic acid (D) at position 238, alanine (A) at position 265, glutamic acid (E) at position 267, glycine (G) at position 271, arginine (R) at position 330, alanine (A) at position 332, or alanine (A) at position 297 (numbering according to EU Index). In some embodiments, the Fc region comprises an aspartic acid (D) at position 238 (EU Index). In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 77. In some embodiments, the HCVR is linked to a heavy chain constant region comprising an amino acid sequence as set forth in SEQ ID NO: 77, with from 0 to 10 amino acid modifications, such as 1, 2, 3, 4, 5, 6, 7, 8, 9 or 10 amino acid modifications. In some embodiments, X at position 121 of SEQ ID NO: 77 is D. In some embodiments, X at position 121 of SEQ ID NO: 77 is P. In some embodiments, X at position 329 of SEQ ID NO: 77 is G. In some embodiments, X at position 329 of SEQ ID NO: 77 is absent. In some embodiments, X at position 330 of SEQ ID NO: 77 is K. In some embodiments, X at position 330 of SEQ ID NO: 77 is absent. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 78. In some embodiments, the HCVR is linked to a heavy chain constant region comprising an amino acid sequence as set forth in SEQ ID NO: 78, with from 0 to 10 amino acid modifications, such as 1, 2, 3, 4, 5, 6, 7, 8, 9 or 10 amino acid modifications. In some embodiments, X at position 108 of SEQ ID NO: 78 is S. In some embodiments, X at position 108 of SEQ ID NO: 78 is P. In some embodiments, X at position 326 of SEQ ID NO: 78 is G. In some embodiments, X at position 326 of SEQ ID NO: 78 is absent. In some embodiments, X at position 327 of SEQ ID NO: 78 is K. In some embodiments, X at position 327 of SEQ ID NO: 78 is absent.

Disclosed herein are methods of treating, preventing, alleviating, or reducing the severity of eczema in a subject in need thereof, comprising administering to the subject a therapeutically effective amount of the antibody disclosed herein, administering to the subject the pharmaceutical composition comprising a therapeutically effective amount of the antibody disclosed herein, or the immunoconjugate, and at least one pharmaceutically acceptable excipient. In some embodiments, eczema comprises atopic dermatitis, contact dermatitis, dyshidrotic eczema, seborrheic dermatitis, neurodermatitis, nummular eczema, stasis dermatitis, or hand eczema. In some embodiments, eczema comprises atopic dermatitis. In some embodiments, the method further comprises administering to the subject one or more therapies selected from antihistamines, corticosteroids, calcineurin inhibitors, antibiotics, and light therapy. In some embodiments, the antihistamine is selected from diphenhydramine, cetirizine, desloratadine, fexofenadine, levocetirizine, and loratadine. In some embodiments, the corticosteroid is selected from cortisone, hydrocortisone, and prednisone. In some embodiments, the corticosteroid is administered as a cream, ointment, or orally. In some embodiments, the calcineurin inhibitor is selected from astagraf xl, cequa, cyclosporine, cyclosporine ophthalmic, elidel, envarsus xr, gengraf, hecoria, lupkynis, neoral, pimecrolimus, prograf, protopic, restasis, sandimmune, tacrolimus, tacrolimus ointment, verkazia, and voclosporin. In some embodiments, the antibiotic is selected from vancomycin, ceftaroline, daptomycin, doxycycline, linezolid, telavancin, tigecycline, and trimethoprim-sulfamethoxazole. In some embodiments, administering the antibody or antigen-binding fragment thereof comprises parenteral, intravenous, oral, subcutaneous, intra-arterial, intracranial, intrathecal, intraperitoneal, intratumoral, topical, intranasal or intramuscular administration. In some embodiments, administering the antibody or antigen-binding fragment thereof comprises intravenous, subcutaneous, or intramuscular administration. In some embodiments, the antibody or antigen-binding fragment thereof comprises a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 70, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some embodiments, the HCVR comprises an amino acid sequence having at least 90%, 95%, 96%, 97%, 98%, 99%, or 100% sequence identity to the amino acid sequence as set forth in SEQ ID NO: 72. In some embodiments, the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 72, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications. In some embodiments, the antibody or antigen-binding fragment thereof comprises a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 5, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some embodiments, the HCVR comprises an amino acid sequence having at least 90%, 95%, 96%, 97%, 98%, 99%, or 100% sequence identity to the amino acid sequence as set forth in SEQ ID NO: 71. In some embodiments, the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 71, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications. In some embodiments, the antibody or antigen-binding fragment thereof comprises a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 92, 41, and 69, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 87, and 91, respectively, each with 0 to 3 amino acid modifications, such as 0, 1, 2, or 3 modifications. In some embodiments, X at position 1 of SEQ ID NO: 69 is M. In some embodiments, X at position 1 of SEQ ID NO: 69 is G. In some embodiments, X at position 1 of SEQ ID NO: 87 is G. In some embodiments, X at position 1 of SEQ ID NO: 87 is L. In some embodiments, X at position 2 of SEQ ID NO: 87 is A. In some embodiments, X at position 2 of SEQ ID NO: 87 is G. In some embodiments, X at position 3 of SEQ ID NO: 87 is S. In some embodiments, X at position 3 of SEQ ID NO: 87 is V. In some embodiments, X at position 7 of SEQ ID NO: 87 is D. In some embodiments, X at position 7 of SEQ ID NO: 87 is S. In some embodiments, X at position 7 of SEQ ID NO: 87 is T. In some embodiments, X at position 8 of SEQ ID NO: 91 is W. In some embodiments, X at position 8 of SEQ ID NO: 91 is F. In some embodiments, X at position 3 of SEQ ID NO: 92 is W. In some embodiments, X at position 3 of SEQ ID NO: 92 is F. In some embodiments, the HCVR comprises an amino acid sequence having at least 90%, 95%, 96%, 97%, 98%, 99%, or 100% sequence identity to the amino acid sequence as set forth in SEQ ID NO: 93. In some embodiments, the LCVR comprises an amino acid sequence having at least 90%, 95%, 96%, 97%, 98%, 99%, or 100% sequence identity to the amino acid sequence as set forth in SEQ ID NO: 94. In some embodiments, the HCVR comprises an amino acid sequence as set forth in SEQ ID NO: 93, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 94, with from 0 to 10 amino acid modifications, such as 1, 2, 3, 4, 5, 6, 7, 8, 9 or 10 amino acid modifications. In some embodiments, X at position 1 of SEQ ID NO: 93 is D. In some embodiments, X at position 1 of SEQ ID NO: 93 is E. In some embodiments, X at position 33 of SEQ ID NO: 93 is W. In some embodiments, X at position 33 of SEQ ID NO: 93 is F. In some embodiments, X at position 99 of SEQ ID NO: 93 is M. In some embodiments, X at position 99 of SEQ ID NO: 93 is G. In some embodiments, X at position 50 of SEQ ID NO: 94 is G. In some embodiments, X at position 50 of SEQ ID NO: 94 is L. In some embodiments, X at position 51 of SEQ ID NO: 94 is A. In some embodiments, X at position 51 of SEQ ID NO: 94 is G. In some embodiments, X at position 52 of SEQ ID NO: 94 is S. In some embodiments, X at position 52 of SEQ ID NO: 94 is V. In some embodiments, X at position 56 of SEQ ID NO: 94 is D. In some embodiments, X at position 56 of SEQ ID NO: 94 is S. In some embodiments, X at position 56 of SEQ ID NO: 94 is T. In some embodiments, X at position 96 of SEQ ID NO: 94 is W. In some embodiments, X at position 96 of SEQ ID NO: 94 is F. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 4, 5, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 7, 8, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 1. In some embodiments, the LCVR comprises an amino acid sequence having at least 90%, 95%, 96%, 97%, 98%, 99%, or 100% sequence identity to the amino acid sequence as set forth in SEQ ID NO: 2. In some embodiments, the HCVR comprises an amino acid sequence as set forth in SEQ ID NO: 1, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 2, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 11, 12, 13, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 14, 15, 16, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 9. In some embodiments, the LCVR comprises an amino acid sequence having at least 90%, 95%, 96%, 97%, 98%, 99%, or 100% sequence identity to the amino acid sequence as set forth in SEQ ID NO: 10. In some embodiments, the HCVR comprises an amino acid sequence as set forth in SEQ ID NO: 9, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 10, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 19, 20, or 21, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 22, 23, or 24, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 17. In some embodiments, the LCVR comprises an amino acid sequence having at least 90%, 95%, 96%, 97%, 98%, 99%, or 100% sequence identity to the amino acid sequence as set forth in SEQ ID NO: 18. In some embodiments, the HCVR comprises an amino acid sequence as set forth in SEQ ID NO: 17, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 18, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 27, 28, or 29, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 30, 31, or 32, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 25. In some embodiments, the LCVR comprises an amino acid sequence having at least 90%, 95%, 96%, 97%, 98%, 99%, or 100% sequence identity to the amino acid sequence as set forth in SEQ ID NO: 26. In some embodiments, the HCVR comprises an amino acid sequence as set forth in SEQ ID NO: 25, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 26, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 35, 36, or 37, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 38, 39, or 40, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 33. In some embodiments, the LCVR comprises an amino acid sequence having at least 90%, 95%, 96%, 97%, 98%, 99%, or 100% sequence identity to the amino acid sequence as set forth in SEQ ID NO: 34. In some embodiments, the HCVR comprises an amino acid sequence as set forth in SEQ ID NO: 33, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 34, with from 0 to 10 amino acid modifications, such as 1, 2, 3, 4, 5, 6, 7, 8, 9 or 10 amino acid modifications. In some embodiments, the LCVR is linked to a light chain constant region comprising an amino acid sequence having at least 90%, 95%, 96%, 97%, 98%, 99%, or 100% sequence identity to the amino acid sequence as set forth in SEQ ID NO: 62. In some embodiments, the light chain variable region is linked to a light chain constant region comprising an amino acid sequence as set forth in SEQ ID NO: 62, with from 0 to 10 amino acid modifications, such as 1, 2, 3, 4, 5, 6, 7, 8, 9 or 10 amino acid modifications. In some embodiments, the light chain variable region is linked to a light chain constant region comprising an amino acid sequence having at least 90%, 95%, 96%, 97%, 98%, 99%, or 100% sequence identity to the amino acid sequence as set forth in SEQ ID NO: 64. In some embodiments, the light chain variable region is linked to a light chain constant region comprising an amino acid sequence as set forth in SEQ ID NO: 64, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 61. In some embodiments, the HCVR is linked to a heavy chain constant region comprising an amino acid sequence as set forth in SEQ ID NO: 61, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 63. In some embodiments, the HCVR is linked to a heavy chain constant region comprising an amino acid sequence as set forth in SEQ ID NO: 63, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 75. In some embodiments, the HCVR is linked to a heavy chain constant region comprising an amino acid sequence as set forth in SEQ ID NO: 75, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 76. In some embodiments, the HCVR is linked to a heavy chain constant region comprising an amino acid sequence as set forth in SEQ ID NO: 76, with from 0 to 10 amino acid modifications, such as 1, 2, 3, 4, 5, 6, 7, 8, 9 or 10 amino acid modifications. In some embodiments, the HCVR is linked to a heavy chain constant region, wherein the heavy chain constant region comprises an Fc region that comprises one or more of the following amino acids: alanine (A) at position 234, alanine (A) at position 235, aspartic acid (D) at position 236, aspartic acid (D) at position 237, aspartic acid (D) at position 238, alanine (A) at position 265, glutamic acid (E) at position 267, glycine (G) at position 271, arginine (R) at position 330, alanine (A) at position 332, or alanine (A) at position 297 (numbering according to EU Index). In some embodiments, the Fc region comprises an aspartic acid (D) at position 238 (EU Index). In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 77. In some embodiments, the HCVR is linked to a heavy chain constant region comprising an amino acid sequence as set forth in SEQ ID NO: 77, with from 0 to 10 amino acid modifications, such as 1, 2, 3, 4, 5, 6, 7, 8, 9 or 10 amino acid modifications. In some embodiments, X at position 121 of SEQ ID NO: 77 is D. In some embodiments, X at position 121 of SEQ ID NO: 77 is P. In some embodiments, X at position 329 of SEQ ID NO: 77 is G. In some embodiments, X at position 329 of SEQ ID NO: 77 is absent. In some embodiments, X at position 330 of SEQ ID NO: 77 is K. In some embodiments, X at position 330 of SEQ ID NO: 77 is absent. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 78. In some embodiments, the HCVR is linked to a heavy chain constant region comprising an amino acid sequence as set forth in SEQ ID NO: 78, with from 0 to 10 amino acid modifications, such as 1, 2, 3, 4, 5, 6, 7, 8, 9 or 10 amino acid modifications. In some embodiments, X at position 108 of SEQ ID NO: 78 is S. In some embodiments, X at position 108 of SEQ ID NO: 78 is P. In some embodiments, X at position 326 of SEQ ID NO: 78 is G. In some embodiments, X at position 326 of SEQ ID NO: 78 is absent. In some embodiments, X at position 327 of SEQ ID NO: 78 is K. In some embodiments, X at position 327 of SEQ ID NO: 78 is absent.

In some cases a method of treating, preventing, alleviating, or reducing the severity of a disease or condition in a subject in need thereof, comprises administering to the subject a therapeutically effective amount of the antibody disclosed herein, administering to the subject the pharmaceutical composition comprising a therapeutically effective amount of the antibody disclosed herein, or the immunoconjugate, and at least one pharmaceutically acceptable excipient, wherein the disease or condition comprises adult-onset Still's disease, alcoholic hepatitis, alcoholic steatohepatitis, alcoholic liver disease, asthma, including allergen-induced asthma, bullous pemphigoid (BP) asthma, non-allergen induced asthma, allergies and allergic conditions such as allergic bronchopulmonary aspergillosis, allergic conjunctivitis, allergic encephalomyelitis, and allergic neuritis, food allergies, allograft rejection, alcoholic steatohepatitis (ASH), ANCA vasculitis, anti-glomerular basement membrane disease (Anti-GBM), antiphospholipid syndrome, aphthous stomatitis, appendicitis, arthritis, autoimmune diseases, atrophic thyroiditis, autoimmune hemolytic anemia (immune pancytopenia, paroxysmal nocturnal hemoglobinuria), autoimmune polyendocrinopathies, autoimmune thrombocytopenia (idiopathic thrombocytopenic purpura, immune-mediated thrombocytopenia), autoimmune hepatitis, pernicious anemia (Addison's disease), and autoimmune thyroid disorders, autoinflammatory diseases, autosomal dominant polyscystic kidney disease (ADPKD), ankylosing spondylitis (AS), acute respiratory distress syndrome (ARDS), Behcet's disease or syndrome, bee sting-induced inflammation, Blau syndrome, bursitis, Barrett's esophagus, bleomycin induced pulmonary fibrosis, bronchiolitis obliterans, cardiac hypertrophy, gluten-sensitive enteropathy (Celiac disease), chemical irritant-induced inflammation, chorioretinitis, chronic atypical neutrophilic dermatosis with lipodystrophy and elevated temperature (CANDLE) syndrome, chronic obstructive pulmonary disease (COPD), chronic pancreatitis, chronic prostatitis, chronic recurrent multifocal osteomyelitis, cicatricial alopecia, colitis, complex regional pain syndrome, chronic intrahepatic or extrahepatic cholestatic disease, conjunctivitis, connective tissue disease, Connective tissue disease-associated interstitial lung disease (CTD-ILD), corneal ulcer, cryopyrin-associated periodic syndromes, cutaneous lupus erythematosus (CLE), cystic fibrosis, deficiency of the interleukin-1 receptor antagonist (DIRA), deficiency of IL36R antagonist (DITRA), dermatitis, diabetic kidney disease (DKD) (diabetic nephropathy), diverticulitis, discoid lupus erythematosus, drug induced delayed type cutaneous allergic reactions, encephalitis, esophagitis, eosinophilic gastrointestinal disorders (EGIDs), such as eosinophilic esophagitis (EoE), eosinophilic gastroenteritis, eosinophilic colitis, familial cold urticarial, familial Mediterranean fever, fistulizing Crohn's disease, giant cell arteritis, glomerulonephritis,g out, gouty arthritis, graft-versus-host disease (GVHD), granulomatous hepatitis, Guillain-Barre syndrome (GBS), Graves' disease, Hashimoto's thyroiditis, Henoch-Schonlein purpura, hidradenitis suppurativa (HS), hyaline membrane disease, hyperactive inflammatory response, hypereosinophilic syndrome (HES), hyperimmunoglobulinemia D with recurrent fever (HIDS), hypersensitivity pneumonitis (HP), immunoglobulin (IgA) nephropathies, IgG4-related disease, immune complex nephritis, immune thrombocytopenic purpura (ITP), inflammation, inflammation of the CNS, inflammatory bowel disease (IBD), inflammatory disease of the respiratory tract (upper or lower) such as inflammatory lung disease, bronchitis, sinusitis, inflammatory ischemic event such as stroke or cardiac arrest, inflammatory liver disease, inflammatory myopathy, inflammatory neuropathy, inflammatory pain, insect bite-induced inflammation, interstitial cystitis, iritis, irritant-induced inflammation, juvenile arthritis, juvenile rheumatoid arthritis, keratitis, kidney transplant rejection, kidney disease, kidney fibrosis, kidney insufficiency, leukocyte adhesion deficiency, Loeffler's syndrome, lupus, lupus nephritis (LN), liver fibrosis, liver steatosis, liver ischemia, lipid and lipoprotein disorders, mast cell activation syndrome, mastocytosis, meningitis, microscopic colitis, mixed connective tissue disease, morphea or morphea variants, Muckle-Wells syndrome (urticaria deafness amyloidosis), mucositis, myelitis, myocarditis, myositis, necrotizing enterocolitis, neonatal onset multisystem inflammatory disease (NOMID), nasal polyps, neovascular glaucoma, neuritis, non-alcoholic fatty liver disease (NAFLD), non-alcoholic steatohepatitis (NASH), non-radiographic axial spondyloarthritis (nr-AxSpA), non-cystic fibrosis bronchiectasis (non-CFB), obstructive or chronic inflammatory disorders of the liver, ocular allergy, optic neuritis, organ transplant rejection, osteoarthritis (OA), otitis, pancreatitis, pancolitis, pelvic inflammatory disease, pemphigus vulgaris (PV), bullous pemphigoid (BP), pericarditis, periodontitis, PFAPA (periodic fever, aphthous stomatitis, pharyngitis, adenitis), plant irritant-induced inflammation, pneumocystis infection, pneumonia, pneumonitis, poison ivy/urushiol oil-induced inflammation, polyarteritis nodosa, polychondritis, polycystic kidney disease (PCKD), polymyalgia rheumatic, polymyositis, pouchitis, proctitis, proctosignmoiditis, psoriatic arthritis (PsA), pulmonary arterial hypertension (PAH), pulmonary fibrosis, pyogenic sterile arthritis, pruritus, reperfusion injury and transplant rejection, primary biliary cirrhosis (PBC), primary sclerosing cholangitis (PSC), Raynaud's syndrome, Reiter's disease, reactive arthritis, renal graft rejection, reperfusion injury, rheumatic carditis, rheumatic diseases, rheumatic fever, rheumatoid arthritis (RA), rhinitis, rhinitis psoriasis, sarcoidosis, Schnitzler syndrome, scleritis, sclerosis, such as systemic sclerosis (SSc), seborrhea, sepsis, septic shock, Sjogren's syndrome, inflammatory skin diseases or conditions, such as acne, alopecia areata, atopic dermatitis, rosacea, eczema, dermatitis, dermatitis endotoxemia, dermatomyositis, stasis dermatitis, Stevens-Johnson syndrome (SJS), skin irritation, skin rash, skin sensitization (contact dermatitis or allergic contact dermatitis), scleroderma, psoriasis, psoriasis vulgaris, psoriatic arthritis, , spinal stenosis, spondyloarthropathies, synovial inflammation, systemic inflammatory response syndrome (SIRS), systemic lupus erythematosus (SLE), systemic mast cell disease (SMCD), systemic vasculitis, systemic-onset juvenile idiopathic arthritis, temporal arteritis, tendinitis, tenosynovitis, thyroditis, transplantation rejection, tubulointerstitial nephritis, tubular disfunction, Takayasu arteritis, toxic epidermal necrolysis, urticaria, uterine fibroids, uveitis, uveoretinitis, vasculitis, vasculitis (NHLBI), vitiligo, Wegener's granulomatosis, acne, acid-induced lung injury, Addison's disease, adrenal hyperplasia, adrenocortical insufficiency, age-related macular degeneration, aging, alcoholic liver disease, Alzheimer's disease, angina pectoris, angiofibroma, anhidrotic ectodermal dysplasia, ascites, aspergillosis, atherosclerosis, atherosclerotic plaques, amyloidosis, amyotrophic lateral sclerosis (ALS), angioedema, acute myocardial infarction; antigen-antibody complex mediated diseases, alpha-1-antitrypsin deficiency; back pain, Bacillus anthracis infection, Bell's palsy, berylliosis, bone pain, burns, bullous pemphigoid, cancer, carpal tunnel syndrome, Castleman's disease, catabolic disorders, cataracts, cerebral aneurysm, complications of organ transplantation, corneal graft neovascularization, cryptococcosis, a non-malignant hyperproliferative disorder; a malignant hyperproliferative disorder; hepatocellular carcinoma; colon adenoma; polyposis; colon adenocarcinoma; breast cancer; pancreatic adenocarcinoma, chronic heart failure, chronic lung disease of prematurity, cardiometabolic syndrome, cardiovascular disease, cutaneous T cell lymphoma, diabetic macular edema, dyslipidemia; endometriosis, endotoxemia, eosinophilic GI disease (EGID), eosinophilic esophagitis (EoE), eosinophilic pneumonias, epicondylitis, epidermolysis bullosa, erythema multiforme, erythroblastopenia, familial amyloidotic polyneuropathy, fetal growth retardation, fibromyalgia, glaucoma, glioblastoma, glomerular disease, gut diseases, growth plate injuries, hair loss, herpes zoster and simplex, hypoplastic and other anemias, head injury, hepatitis A, B, C, D, and E, herpes; headache, hearing loss, heart disease, hemangioma, hemophilic joints, hereditary periodic fever syndrome, heritable disorders of connective tissue, Hodgkin's disease, Huntington's disease, hyperammonemia, hypercalcemia, hypercholesterolemia, hemolytic anemia, hepatitis, hip replacement, hypertropic bone formation, hypersensitivity pneumonia, hereditary fructose intolerance, hypertension, hyperuricemia, idiopathic demyelinating polyneuropathy, infectious diseases including viral diseases such as AIDS (HIV infection), ichthyosis, incontinentia pigmenti (IP, Bloch-Siemens syndrome), idiopathic thrombocytopenic purpura, infectious mononucleosis, ischemia/reperfusion, insulin resistance, joint replacement, kidney injury caused by parasitic infections, leptospirosis, lichen sclerosus (LS), lichen planus, Lambert-Eaton myasthenic syndrome, Lyme disease, liver failure, including acute liver failure, muscle wasting, muscular dystrophy, Marfan syndrome (MFS), meningioma, mesothelioma, multiple organ injury syndrome, myasthenia gravis (MG), myelodysplastic syndrome, metabolic syndrome, multiple sclerosis, nephrotic syndrome, neuropathological diseases, nuclear factor-kappa B essential modulator (NEMO) deficiency syndrome, obesity, Osler-Weber syndrome, osteogenesis imperfecta, osteonecrosis, osteoporosis, pachyonychia congenita, Paget's disease, Paget's disease of bone, Parkinson's disease, periodic fever, pertussis, primary pulmonary hypertension, pyoderma gangrenosum, pyogenic granuloma retrolental fibroplasias, peritoneal endometriosis, Prurigo nodularis, psychosocial stress diseases, pulmonary disease, pulmonary hypertension, respiratory distress syndrome, renal disease, retinal disease, retrolental fibroplasia, renal transplant rejection, renal protection against drugs inducing Fanconi's syndrome, respiratory tract illness caused by respiratory syncytial virus, rhinosinusitis; radiation induced fibrosis, sarcoidosis, severe pain, sleep apnea, scoliosis, sickle cell anemia, sports injuries, sprains and strains, sunburn, spinal cord injury, Sezary syndrome, silica-induced disease (Silicosis), subarachnoid hemorrhage, tuberculosis, tumor necrosis factor (TNF) receptor associated periodic syndrome (TRAPS), thrombosis; traumatic brain injury, tissue transplant, complications from type 1 or type 2 diabetes, toxoplasmosis, thrombocytopenia, trachoma, vascular restenosis, ventilator induced lung injury, Whipple's disease, and 2,8-dihydroxyadenine nephropathy. In some embodiments, administering the antibody or antigen-binding fragment thereof comprises parenteral, intravenous, oral, subcutaneous, intra-arterial, intracranial, intrathecal, intraperitoneal, intratumoral, topical, intranasal or intramuscular administration. In some embodiments, administering the antibody or antigen-binding fragment thereof comprises intravenous, subcutaneous, or intramuscular administration. In some embodiments, the antibody or antigen-binding fragment thereof comprises a heavy chain variable region (HCVR) and a light chain variable region (LCVR), wherein: (a) the HCVR comprises heavy chain complementarity determining region 1 (CDRH1), CDRH2, and CDRH3 comprising the sequence as set forth in SEQ ID NOs: 3, 41, and 70, respectively, each with 0 to 3 amino acid modifications, such as 0, 1, 2, or 3 modifications; and (b) the LCVR comprises light chain complementarity determining region 1 (CDRL1), CDRL2, and CDRL3 comprising the sequence as set forth in SEQ ID NOs: 6, 67, and 8, respectively, each with 0 to 3 amino acid modifications, such as 0, 1, 2, or 3 modifications. In some embodiments, the HCVR comprises an amino acid sequence having at least 90%, 95%, 96%, 97%, 98%, 99%, or 100% sequence identity to the amino acid sequence as set forth in SEQ ID NO: 72. In some embodiments, the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 72, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications. In some embodiments, the antibody or antigen-binding fragment thereof comprises a heavy chain variable region (HCVR) and a light chain variable region (LCVR), wherein: (a) the HCVR comprises heavy chain complementarity determining region 1 (CDRH1), CDRH2, and CDRH3 comprising the sequence as set forth in SEQ ID NOs: 3, 41, and 5, respectively, each with 0 to 3 amino acid modifications, such as 0, 1, 2, or 3 modifications; and (b) the LCVR comprises light chain complementarity determining region 1 (CDRL1), CDRL2, and CDRL3 comprising the sequence as set forth in SEQ ID NOs: 6, 67, and 8, respectively, each with 0 to 3 amino acid modifications, such as 0, 1, 2, or 3 modifications. In some embodiments, the HCVR comprises an amino acid sequence having at least 90%, 95%, 96%, 97%, 98%, 99%, or 100% sequence identity to the amino acid sequence as set forth in SEQ ID NO: 71. In some embodiments, the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 71, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications. In some embodiments, the antibody or antigen-binding fragment thereof comprises a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 92, 41, and 69, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 87, and 91, respectively, each with 0 to 3 amino acid modifications, such as 0, 1, 2, or 3 modifications. In some embodiments, X at position 1 of SEQ ID NO: 69 is M. In some embodiments, X at position 1 of SEQ ID NO: 69 is G. In some embodiments, X at position 1 of SEQ ID NO: 87 is G. In some embodiments, X at position 1 of SEQ ID NO: 87 is L. In some embodiments, X at position 2 of SEQ ID NO: 87 is A. In some embodiments, X at position 2 of SEQ ID NO: 87 is G. In some embodiments, X at position 3 of SEQ ID NO: 87 is S. In some embodiments, X at position 3 of SEQ ID NO: 87 is V. In some embodiments, X at position 7 of SEQ ID NO: 87 is D. In some embodiments, X at position 7 of SEQ ID NO: 87 is S. In some embodiments, X at position 7 of SEQ ID NO: 87 is T. In some embodiments, X at position 8 of SEQ ID NO: 91 is W. In some embodiments, X at position 8 of SEQ ID NO: 91 is F. In some embodiments, X at position 3 of SEQ ID NO: 92 is W. In some embodiments, X at position 3 of SEQ ID NO: 92 is F. In some embodiments, the HCVR comprises an amino acid sequence having at least 90%, 95%, 96%, 97%, 98%, 99%, or 100% sequence identity to the amino acid sequence as set forth in SEQ ID NO: 93. In some embodiments, the LCVR comprises an amino acid sequence having at least 90%, 95%, 96%, 97%, 98%, 99%, or 100% sequence identity to the amino acid sequence as set forth in SEQ ID NO: 94. In some embodiments, the HCVR comprises an amino acid sequence as set forth in SEQ ID NO: 93, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 94, with from 0 to 10 amino acid modifications, such as 1, 2, 3, 4, 5, 6, 7, 8, 9 or 10 amino acid modifications. In some embodiments, X at position 1 of SEQ ID NO: 93 is D. In some embodiments, X at position 1 of SEQ ID NO: 93 is E. In some embodiments, X at position 33 of SEQ ID NO: 93 is W. In some embodiments, X at position 33 of SEQ ID NO: 93 is F. In some embodiments, X at position 99 of SEQ ID NO: 93 is M. In some embodiments, X at position 99 of SEQ ID NO: 93 is G. In some embodiments, X at position 50 of SEQ ID NO: 94 is G. In some embodiments, X at position 50 of SEQ ID NO: 94 is L. In some embodiments, X at position 51 of SEQ ID NO: 94 is A. In some embodiments, X at position 51 of SEQ ID NO: 94 is G. In some embodiments, X at position 52 of SEQ ID NO: 94 is S. In some embodiments, X at position 52 of SEQ ID NO: 94 is V. In some embodiments, X at position 56 of SEQ ID NO: 94 is D. In some embodiments, X at position 56 of SEQ ID NO: 94 is S. In some embodiments, X at position 56 of SEQ ID NO: 94 is T. In some embodiments, X at position 96 of SEQ ID NO: 94 is W. In some embodiments, X at position 96 of SEQ ID NO: 94 is F. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 4, 5, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 7, 8, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 1. In some embodiments, the LCVR comprises an amino acid sequence having at least 90%, 95%, 96%, 97%, 98%, 99%, or 100% sequence identity to the amino acid sequence as set forth in SEQ ID NO: 2. In some embodiments, the HCVR comprises an amino acid sequence as set forth in SEQ ID NO: 1, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 2, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 11, 12, 13, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 14, 15, 16, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 9. In some embodiments, the LCVR comprises an amino acid sequence having at least 90%, 95%, 96%, 97%, 98%, 99%, or 100% sequence identity to the amino acid sequence as set forth in SEQ ID NO: 10. In some embodiments, the HCVR comprises an amino acid sequence as set forth in SEQ ID NO: 9, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 10, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 19, 20, or 21, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 22, 23, or 24, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 17. In some embodiments, the LCVR comprises an amino acid sequence having at least 90%, 95%, 96%, 97%, 98%, 99%, or 100% sequence identity to the amino acid sequence as set forth in SEQ ID NO: 18. In some embodiments, the HCVR comprises an amino acid sequence as set forth in SEQ ID NO: 17, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 18, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 27, 28, or 29, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 30, 31, or 32, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 25. In some embodiments, the LCVR comprises an amino acid sequence having at least 90%, 95%, 96%, 97%, 98%, 99%, or 100% sequence identity to the amino acid sequence as set forth in SEQ ID NO: 26. In some embodiments, the HCVR comprises an amino acid sequence as set forth in SEQ ID NO: 25, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 26, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 35, 36, or 37, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 38, 39, or 40, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 33. In some embodiments, the LCVR comprises an amino acid sequence having at least 90%, 95%, 96%, 97%, 98%, 99%, or 100% sequence identity to the amino acid sequence as set forth in SEQ ID NO: 34. In some embodiments, the HCVR comprises an amino acid sequence as set forth in SEQ ID NO: 33, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 34, with from 0 to 10 amino acid modifications, such as 1, 2, 3, 4, 5, 6, 7, 8, 9 or 10 amino acid modifications. In some embodiments, the LCVR is linked to a light chain constant region comprising an amino acid sequence having at least 90%, 95%, 96%, 97%, 98%, 99%, or 100% sequence identity to the amino acid sequence as set forth in SEQ ID NO: 62. In some embodiments, the light chain variable region is linked to a light chain constant region comprising an amino acid sequence as set forth in SEQ ID NO: 62, with from 0 to 10 amino acid modifications, such as 1, 2, 3, 4, 5, 6, 7, 8, 9 or 10 amino acid modifications. In some embodiments, the light chain variable region is linked to a light chain constant region comprising an amino acid sequence having at least 90%, 95%, 96%, 97%, 98%, 99%, or 100% sequence identity to the amino acid sequence as set forth in SEQ ID NO: 64. In some embodiments, the light chain variable region is linked to a light chain constant region comprising an amino acid sequence as set forth in SEQ ID NO: 64, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 61. In some embodiments, the HCVR is linked to a heavy chain constant region comprising an amino acid sequence as set forth in SEQ ID NO: 61, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 63. In some embodiments, the HCVR is linked to a heavy chain constant region comprising an amino acid sequence as set forth in SEQ ID NO: 63, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 75. In some embodiments, the HCVR is linked to a heavy chain constant region comprising an amino acid sequence as set forth in SEQ ID NO: 75, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 76. In some embodiments, the HCVR is linked to a heavy chain constant region comprising an amino acid sequence as set forth in SEQ ID NO: 76, with from 0 to 10 amino acid modifications, such as 1, 2, 3, 4, 5, 6, 7, 8, 9 or 10 amino acid modifications. In some embodiments, the HCVR is linked to a heavy chain constant region, wherein the heavy chain constant region comprises an Fc region that comprises one or more of the following amino acids: alanine (A) at position 234, alanine (A) at position 235, aspartic acid (D) at position 236, aspartic acid (D) at position 237, aspartic acid (D) at position 238, alanine (A) at position 265, glutamic acid (E) at position 267, glycine (G) at position 271, arginine (R) at position 330, alanine (A) at position 332, or alanine (A) at position 297 (numbering according to EU Index). In some embodiments, the Fc region comprises an aspartic acid (D) at position 238 (EU Index). In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 77. In some embodiments, the HCVR is linked to a heavy chain constant region comprising an amino acid sequence as set forth in SEQ ID NO: 77, with from 0 to 10 amino acid modifications, such as 1, 2, 3, 4, 5, 6, 7, 8, 9 or 10 amino acid modifications. In some embodiments, X at position 121 of SEQ ID NO: 77 is D. In some embodiments, X at position 121 of SEQ ID NO: 77 is P. In some embodiments, X at position 329 of SEQ ID NO: 77 is G. In some embodiments, X at position 329 of SEQ ID NO: 77 is absent. In some embodiments, X at position 330 of SEQ ID NO: 77 is K. In some embodiments, X at position 330 of SEQ ID NO: 77 is absent. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 78. In some embodiments, the HCVR is linked to a heavy chain constant region comprising an amino acid sequence as set forth in SEQ ID NO: 78, with from 0 to 10 amino acid modifications, such as 1, 2, 3, 4, 5, 6, 7, 8, 9 or 10 amino acid modifications. In some embodiments, X at position 108 of SEQ ID NO: 78 is S. In some embodiments, X at position 108 of SEQ ID NO: 78 is P. In some embodiments, X at position 326 of SEQ ID NO: 78 is G. In some embodiments, X at position 326 of SEQ ID NO: 78 is absent. In some embodiments, X at position 327 of SEQ ID NO: 78 is K. In some embodiments, X at position 327 of SEQ ID NO: 78 is absent.

In some cases a method of treating, preventing, alleviating, or reducing the severity of a disease or condition in a subject in need thereof, comprises administering to the subject a therapeutically effective amount of the antibody disclosed herein, administering to the subject the pharmaceutical composition comprising a therapeutically effective amount of the antibody disclosed herein, or the immunoconjugate, and at least one pharmaceutically acceptable excipient, wherein the disease or condition comprises a rheumatological condition, gastrointestinal disease or condition, pulmonary disease or condition, hepatological disease or condition, nephrological disease or condition, or dermatological disease or condition. In some embodiments, the rheumatological condition or disease is selected from rheumatoid arthritis (RA), systemic lupus erythematosus (SLE), lupus nephritis (LN), osteoarthritis (OA), Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, Takayasu arteritis, cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the rheumatological condition or disease is selected from rheumatoid arthritis (RA), systemic lupus erythematosus (SLE), lupus nephritis (LN), and osteoarthritis (OA). In some embodiments, the rheumatological condition or disease is selected Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, and Takayasu arteritis. In some embodiments, the rheumatological condition or disease is selected cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the gastrointestinal disease or condition is selected from ulcerative colitis (UC), Crohn's disease (CD), eosinophilic gastrointestinal disorders (EGIDs), microscopic colitis, ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the gastrointestinal disease or condition is selected from ulcerative colitis (UC) and Crohn's disease (CD). In some embodiments, the gastrointestinal disease or condition is selected from eosinophilic gastrointestinal disorders (EGIDs) and microscopic colitis. In some embodiments, the EGID is selected from eosinophilic esophagitis (EoE), eosinophilic gastroenteritis, and eosinophilic colitis. In some embodiments, the microscopic colitis is selected from collagenous colitis and lymphocytic colitis. In some embodiments, the gastrointestinal disease is selected from ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the pulmonary disease or condition is selected from idiopathic pulmonary fibrosis (IPF), interstitial lung disease (ILD), acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the pulmonary disease or condition is selected from idiopathic pulmonary fibrosis (IPF) and interstitial lung disease (ILD). In some embodiments, the pulmonary disease or condition is selected from acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the hepatological disease or condition is selected from non-alcoholic steatohepatitis (NASH), primary sclerosing cholangitis (PSC), primary biliary cirrhosis (PBC), autoimmune hepatitis, alcoholic steatohepatitis (ASH), alcoholic hepatitis, chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the hepatological disease or condition is non-alcoholic steatohepatitis (NASH). In some embodiments, the hepatological disease or condition is selected from primary sclerosing cholangitis (PSC), primary biliary cirrhosis (PBC), autoimmune hepatitis, alcoholic steatohepatitis (ASH), and alcoholic hepatitis. In some embodiments, the hepatological disease or condition is selected from chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the nephrological disease or condition is selected from diabetic kidney disease (DKD) (diabetic nephropathy), chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the nephrological disease or condition is diabetic kidney disease (DKD) (diabetic nephropathy). In some embodiments, the nephrological disease or condition is selected from chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the dermatological disease or condition is selected from atopic dermatitis (AD), contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea. In some embodiments, the dermatological disease or condition is atopic dermatitis (AD). In some embodiments, the dermatological disease or condition is selected from contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea. In some embodiments, administering the antibody or antigen-binding fragment thereof comprises parenteral, intravenous, oral, subcutaneous, intra-arterial, intracranial, intrathecal, intraperitoneal, intratumoral, topical, intranasal or intramuscular administration. In some embodiments, administering the antibody or antigen-binding fragment thereof comprises intravenous, subcutaneous, or intramuscular administration. In some embodiments, the antibody or antigen-binding fragment thereof comprises a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 70, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some embodiments, the HCVR comprises an amino acid sequence having at least 90%, 95%, 96%, 97%, 98%, 99%, or 100% sequence identity to the amino acid sequence as set forth in SEQ ID NO: 72. In some embodiments, the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 72, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications. In some embodiments, the antibody or antigen-binding fragment thereof comprises a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 5, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some embodiments, the HCVR comprises an amino acid sequence having at least 90%, 95%, 96%, 97%, 98%, 99%, or 100% sequence identity to the amino acid sequence as set forth in SEQ ID NO: 71. In some embodiments, the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 71, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications. In some embodiments, the antibody or antigen-binding fragment thereof comprises a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 92, 41, and 69, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 87, and 91, respectively, each with 0 to 3 amino acid modifications, such as 0, 1, 2, or 3 modifications. In some embodiments, X at position 1 of SEQ ID NO: 69 is M. In some embodiments, X at position 1 of SEQ ID NO: 69 is G. In some embodiments, X at position 1 of SEQ ID NO: 87 is G. In some embodiments, X at position 1 of SEQ ID NO: 87 is L. In some embodiments, X at position 2 of SEQ ID NO: 87 is A. In some embodiments, X at position 2 of SEQ ID NO: 87 is G. In some embodiments, X at position 3 of SEQ ID NO: 87 is S. In some embodiments, X at position 3 of SEQ ID NO: 87 is V. In some embodiments, X at position 7 of SEQ ID NO: 87 is D. In some embodiments, X at position 7 of SEQ ID NO: 87 is S. In some embodiments, X at position 7 of SEQ ID NO: 87 is T. In some embodiments, X at position 8 of SEQ ID NO: 91 is W. In some embodiments, X at position 8 of SEQ ID NO: 91 is F. In some embodiments, X at position 3 of SEQ ID NO: 92 is W. In some embodiments, X at position 3 of SEQ ID NO: 92 is F. In some embodiments, the HCVR comprises an amino acid sequence having at least 90%, 95%, 96%, 97%, 98%, 99%, or 100% sequence identity to the amino acid sequence as set forth in SEQ ID NO: 93. In some embodiments, the LCVR comprises an amino acid sequence having at least 90%, 95%, 96%, 97%, 98%, 99%, or 100% sequence identity to the amino acid sequence as set forth in SEQ ID NO: 94. In some embodiments, the HCVR comprises an amino acid sequence as set forth in SEQ ID NO: 93, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 94, with from 0 to 10 amino acid modifications, such as 1, 2, 3, 4, 5, 6, 7, 8, 9 or 10 amino acid modifications. In some embodiments, X at position 1 of SEQ ID NO: 93 is D. In some embodiments, X at position 1 of SEQ ID NO: 93 is E. In some embodiments, X at position 33 of SEQ ID NO: 93 is W. In some embodiments, X at position 33 of SEQ ID NO: 93 is F. In some embodiments, X at position 99 of SEQ ID NO: 93 is M. In some embodiments, X at position 99 of SEQ ID NO: 93 is G. In some embodiments, X at position 50 of SEQ ID NO: 94 is G. In some embodiments, X at position 50 of SEQ ID NO: 94 is L. In some embodiments, X at position 51 of SEQ ID NO: 94 is A. In some embodiments, X at position 51 of SEQ ID NO: 94 is G. In some embodiments, X at position 52 of SEQ ID NO: 94 is S. In some embodiments, X at position 52 of SEQ ID NO: 94 is V. In some embodiments, X at position 56 of SEQ ID NO: 94 is D. In some embodiments, X at position 56 of SEQ ID NO: 94 is S. In some embodiments, X at position 56 of SEQ ID NO: 94 is T. In some embodiments, X at position 96 of SEQ ID NO: 94 is W. In some embodiments, X at position 96 of SEQ ID NO: 94 is F. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 4, 5, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 7, 8, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 1. In some embodiments, the LCVR comprises an amino acid sequence having at least 90%, 95%, 96%, 97%, 98%, 99%, or 100% sequence identity to the amino acid sequence as set forth in SEQ ID NO: 2. In some embodiments, the HCVR comprises an amino acid sequence as set forth in SEQ ID NO: 1, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 2, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 11, 12, 13, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 14, 15, 16, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 9. In some embodiments, the LCVR comprises an amino acid sequence having at least 90%, 95%, 96%, 97%, 98%, 99%, or 100% sequence identity to the amino acid sequence as set forth in SEQ ID NO: 10. In some embodiments, the HCVR comprises an amino acid sequence as set forth in SEQ ID NO: 9, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 10, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 19, 20, or 21, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 22, 23, or 24, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 17. In some embodiments, the LCVR comprises an amino acid sequence having at least 90%, 95%, 96%, 97%, 98%, 99%, or 100% sequence identity to the amino acid sequence as set forth in SEQ ID NO: 18. In some embodiments, the HCVR comprises an amino acid sequence as set forth in SEQ ID NO: 17, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 18, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 27, 28, or 29, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 30, 31, or 32, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 25. In some embodiments, the LCVR comprises an amino acid sequence having at least 90%, 95%, 96%, 97%, 98%, 99%, or 100% sequence identity to the amino acid sequence as set forth in SEQ ID NO: 26. In some embodiments, the HCVR comprises an amino acid sequence as set forth in SEQ ID NO: 25, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 26, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 35, 36, or 37, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 38, 39, or 40, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 33. In some embodiments, the LCVR comprises an amino acid sequence having at least 90%, 95%, 96%, 97%, 98%, 99%, or 100% sequence identity to the amino acid sequence as set forth in SEQ ID NO: 34. In some embodiments, the HCVR comprises an amino acid sequence as set forth in SEQ ID NO: 33, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 34, with from 0 to 10 amino acid modifications, such as 1, 2, 3, 4, 5, 6, 7, 8, 9 or 10 amino acid modifications. In some embodiments, the LCVR is linked to a light chain constant region comprising an amino acid sequence having at least 90%, 95%, 96%, 97%, 98%, 99%, or 100% sequence identity to the amino acid sequence as set forth in SEQ ID NO: 62. In some embodiments, the light chain variable region is linked to a light chain constant region comprising an amino acid sequence as set forth in SEQ ID NO: 62, with from 0 to 10 amino acid modifications, such as 1, 2, 3, 4, 5, 6, 7, 8, 9 or 10 amino acid modifications. In some embodiments, the light chain variable region is linked to a light chain constant region comprising an amino acid sequence having at least 90%, 95%, 96%, 97%, 98%, 99%, or 100% sequence identity to the amino acid sequence as set forth in SEQ ID NO: 64. In some embodiments, the light chain variable region is linked to a light chain constant region comprising an amino acid sequence as set forth in SEQ ID NO: 64, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 61. In some embodiments, the HCVR is linked to a heavy chain constant region comprising an amino acid sequence as set forth in SEQ ID NO: 61, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 63. In some embodiments, the HCVR is linked to a heavy chain constant region comprising an amino acid sequence as set forth in SEQ ID NO: 63, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 75. In some embodiments, the HCVR is linked to a heavy chain constant region comprising an amino acid sequence as set forth in SEQ ID NO: 75, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 76. In some embodiments, the HCVR is linked to a heavy chain constant region comprising an amino acid sequence as set forth in SEQ ID NO: 76, with from 0 to 10 amino acid modifications, such as 1, 2, 3, 4, 5, 6, 7, 8, 9 or 10 amino acid modifications. In some embodiments, the HCVR is linked to a heavy chain constant region, wherein the heavy chain constant region comprises an Fc region that comprises one or more of the following amino acids: alanine (A) at position 234, alanine (A) at position 235, aspartic acid (D) at position 236, aspartic acid (D) at position 237, aspartic acid (D) at position 238, alanine (A) at position 265, glutamic acid (E) at position 267, glycine (G) at position 271, arginine (R) at position 330, alanine (A) at position 332, or alanine (A) at position 297 (numbering according to EU Index). In some embodiments, the Fc region comprises an aspartic acid (D) at position 238 (EU Index). In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 77. In some embodiments, the HCVR is linked to a heavy chain constant region comprising an amino acid sequence as set forth in SEQ ID NO: 77, with from 0 to 10 amino acid modifications, such as 1, 2, 3, 4, 5, 6, 7, 8, 9 or 10 amino acid modifications. In some embodiments, X at position 121 of SEQ ID NO: 77 is D. In some embodiments, X at position 121 of SEQ ID NO: 77 is P. In some embodiments, X at position 329 of SEQ ID NO: 77 is G. In some embodiments, X at position 329 of SEQ ID NO: 77 is absent. In some embodiments, X at position 330 of SEQ ID NO: 77 is K. In some embodiments, X at position 330 of SEQ ID NO: 77 is absent. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 78. In some embodiments, the HCVR is linked to a heavy chain constant region comprising an amino acid sequence as set forth in SEQ ID NO: 78, with from 0 to 10 amino acid modifications, such as 1, 2, 3, 4, 5, 6, 7, 8, 9 or 10 amino acid modifications. In some embodiments, X at position 108 of SEQ ID NO: 78 is S. In some embodiments, X at position 108 of SEQ ID NO: 78 is P. In some embodiments, X at position 326 of SEQ ID NO: 78 is G. In some embodiments, X at position 326 of SEQ ID NO: 78 is absent. In some embodiments, X at position 327 of SEQ ID NO: 78 is K. In some embodiments, X at position 327 of SEQ ID NO: 78 is absent.

In some embodiments, the present disclosure provides a method of treating, preventing, alleviating, or reducing the severity of cancer in a mammal in need thereof, comprising administering to the mammal a therapeutically effective amount of an antibody of the present disclosure. In some cases, the cancer is hepatocellular carcinoma. In other cases, the cancer is acute myeloid leukemia, thymus, brain, lung, squamous cell, skin, eye, retinoblastoma, intraocular melanoma, oral cavity and oropharyngeal, bladder, gastric, stomach, pancreatic, bladder, breast, cervical, head, neck, renal, kidney, liver, ovarian, prostate, colorectal, esophageal, testicular, gynecological, thyroid, CNS, PNS, AIDS related (e.g. Lymphoma and Kaposi's Sarcoma) or Viral-Induced cancer. In some embodiments, administering the antibody or antigen-binding fragment thereof comprises parenteral, intravenous, oral, subcutaneous, intra-arterial, intracranial, intrathecal, intraperitoneal, intratumoral, topical, intranasal or intramuscular administration. In some embodiments, administering the antibody or antigen-binding fragment thereof comprises intravenous, subcutaneous, or intramuscular administration. In some embodiments, the antibody or antigen-binding fragment thereof comprises a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 70, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some embodiments, the HCVR comprises an amino acid sequence having at least 90%, 95%, 96%, 97%, 98%, 99%, or 100% sequence identity to the amino acid sequence as set forth in SEQ ID NO: 72. In some embodiments, the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 72, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications. In some embodiments, the antibody or antigen-binding fragment thereof comprises a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 5, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some embodiments, the HCVR comprises an amino acid sequence having at least 90%, 95%, 96%, 97%, 98%, 99%, or 100% sequence identity to the amino acid sequence as set forth in SEQ ID NO: 71. In some embodiments, the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 71, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 4, 5, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 7, 8, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 1. In some embodiments, the LCVR comprises an amino acid sequence having at least 90%, 95%, 96%, 97%, 98%, 99%, or 100% sequence identity to the amino acid sequence as set forth in SEQ ID NO: 2. In some embodiments, the HCVR comprises an amino acid sequence as set forth in SEQ ID NO: 1, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 2, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 11, 12, 13, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 14, 15, 16, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 9. In some embodiments, the LCVR comprises an amino acid sequence having at least 90%, 95%, 96%, 97%, 98%, 99%, or 100% sequence identity to the amino acid sequence as set forth in SEQ ID NO: 10. In some embodiments, the HCVR comprises an amino acid sequence as set forth in SEQ ID NO: 9, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 10, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 19, 20, or 21, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 22, 23, or 24, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 17. In some embodiments, the LCVR comprises an amino acid sequence having at least 90%, 95%, 96%, 97%, 98%, 99%, or 100% sequence identity to the amino acid sequence as set forth in SEQ ID NO: 18. In some embodiments, the HCVR comprises an amino acid sequence as set forth in SEQ ID NO: 17, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 18, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 27, 28, or 29, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 30, 31, or 32, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 25. In some embodiments, the LCVR comprises an amino acid sequence having at least 90%, 95%, 96%, 97%, 98%, 99%, or 100% sequence identity to the amino acid sequence as set forth in SEQ ID NO: 26. In some embodiments, the HCVR comprises an amino acid sequence as set forth in SEQ ID NO: 25, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 26, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 35, 36, or 37, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 38, 39, or 40, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 33. In some embodiments, the LCVR comprises an amino acid sequence having at least 90%, 95%, 96%, 97%, 98%, 99%, or 100% sequence identity to the amino acid sequence as set forth in SEQ ID NO: 34. In some embodiments, the HCVR comprises an amino acid sequence as set forth in SEQ ID NO: 33, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 34, with from 0 to 10 amino acid modifications, such as 1, 2, 3, 4, 5, 6, 7, 8, 9 or 10 amino acid modifications. In some embodiments, the LCVR is linked to a light chain constant region comprising an amino acid sequence having at least 90%, 95%, 96%, 97%, 98%, 99%, or 100% sequence identity to the amino acid sequence as set forth in SEQ ID NO: 62. In some embodiments, the light chain variable region is linked to a light chain constant region comprising an amino acid sequence as set forth in SEQ ID NO: 62, with from 0 to 10 amino acid modifications, such as 1, 2, 3, 4, 5, 6, 7, 8, 9 or 10 amino acid modifications. In some embodiments, the light chain variable region is linked to a light chain constant region comprising an amino acid sequence having at least 90%, 95%, 96%, 97%, 98%, 99%, or 100% sequence identity to the amino acid sequence as set forth in SEQ ID NO: 64. In some embodiments, the light chain variable region is linked to a light chain constant region comprising an amino acid sequence as set forth in SEQ ID NO: 64, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 61. In some embodiments, the HCVR is linked to a heavy chain constant region comprising an amino acid sequence as set forth in SEQ ID NO: 61, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 63. In some embodiments, the HCVR is linked to a heavy chain constant region comprising an amino acid sequence as set forth in SEQ ID NO: 63, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 75. In some embodiments, the HCVR is linked to a heavy chain constant region comprising an amino acid sequence as set forth in SEQ ID NO: 75, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 76. In some embodiments, the HCVR is linked to a heavy chain constant region comprising an amino acid sequence as set forth in SEQ ID NO: 76, with from 0 to 10 amino acid modifications, such as 1, 2, 3, 4, 5, 6, 7, 8, 9 or 10 amino acid modifications. In some embodiments, the HCVR is linked to a heavy chain constant region, wherein the heavy chain constant region comprises an Fc region that comprises one or more of the following amino acids: alanine (A) at position 234, alanine (A) at position 235, aspartic acid (D) at position 236, aspartic acid (D) at position 237, aspartic acid (D) at position 238, alanine (A) at position 265, glutamic acid (E) at position 267, glycine (G) at position 271, arginine (R) at position 330, alanine (A) at position 332, or alanine (A) at position 297 (numbering according to EU Index). In some embodiments, the Fc region comprises an aspartic acid (D) at position 238 (EU Index). In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 77. In some embodiments, the HCVR is linked to a heavy chain constant region comprising an amino acid sequence as set forth in SEQ ID NO: 77, with from 0 to 10 amino acid modifications, such as 1, 2, 3, 4, 5, 6, 7, 8, 9 or 10 amino acid modifications. In some embodiments, X at position 121 of SEQ ID NO: 77 is D. In some embodiments, X at position 121 of SEQ ID NO: 77 is P. In some embodiments, X at position 329 of SEQ ID NO: 77 is G. In some embodiments, X at position 329 of SEQ ID NO: 77 is absent. In some embodiments, X at position 330 of SEQ ID NO: 77 is K. In some embodiments, X at position 330 of SEQ ID NO: 77 is absent. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 78. In some embodiments, the HCVR is linked to a heavy chain constant region comprising an amino acid sequence as set forth in SEQ ID NO: 78, with from 0 to 10 amino acid modifications, such as 1, 2, 3, 4, 5, 6, 7, 8, 9 or 10 amino acid modifications. In some embodiments, X at position 108 of SEQ ID NO: 78 is S. In some embodiments, X at position 108 of SEQ ID NO: 78 is P. In some embodiments, X at position 326 of SEQ ID NO: 78 is G. In some embodiments, X at position 326 of SEQ ID NO: 78 is absent. In some embodiments, X at position 327 of SEQ ID NO: 78 is K. In some embodiments, X at position 327 of SEQ ID NO: 78 is absent.

In some embodiments, the subject to be treated is a mammal, such as a human. In some embodiments, the subject to be treated is a human. In other cases, the mammal is a mouse, a rat, a cat, a dog, a rabbit, a pig, a sheep, a horse, a bovine, a goat, a gerbil, a hamster, a guinea pig, a monkey or any other mammal. Many such mammals may be subjects that are known to the art as preclinical models for certain diseases or disorders, including inflammatory diseases, solid tumors and/or other cancers (e.g., Talmadge et al., 2007 Am. J. Pathol. 170:793; Kerbel, 2003 Canc. Biol. Therap. 2(4 Suppl 1):S134; Man et al., 2007 Canc. Met. Rev. 26:737; Cespedes et al., 2006 Clin. TransL Oncol. 8:318).

In another aspect, the disclosure provides methods of using the CD200R antibody of the present disclosure to treat diseases or conditions in a mammal in conjunction with a second agent. The second agent could be administered together with, before, or after the antibody. In some embodiments, the second agent is an agent that acts to relieve the symptoms of inflammatory conditions described herein. Anti-inflammatory agents include non-steroidal anti-inflammatory drugs (NSAIDs) and corticosteroids. NSAIDs include but are not limited to, salicylates, such as acetylsalicylic acid; diflunisal, salicylic acid, and salsalate; propionic acid derivatives, such as ibuprofen; naproxen; dexibuprofen, dexketoprofen, flurbiprofen, oxaprozin, fenoprofen, loxoprofen, and ketoprofen; acetic acid derivatives, such as indomethacin, diclofenac, tolmetin, aceclofenac, sulindac, nabumetone, etodolac, and ketorolac; enolic acid derivatives, such as piroxicam, lornoxicam, meloxicam, isoxicam, tenoxicam, phenylbutazone, and droxicam; anthranilic acid derivatives, such as mefenamic acid, flufenamic acid, meclofenamic acid, and tolfenamic acid; selective COX-2 inhibitors, such as celecoxib, lumiracoxib, rofecoxib, etoricoxib, valdecoxib, firocoxib, and parecoxib; sulfonanilides, such as nimesulide; and others such as clonixin, and licofelone. Corticosteroids include but are not limited to, cortisone, dexamethasone, hydrocortisone, methylprednisolone, prednisone, and prednisolone.

In some embodiments, the second agent is an immunosuppressant. The immunosuppressants that can be used in combination with the subject antibody include but are not limited to hydroxychloroquine, sulfasalazine, leflunomide, etanercept, infliximab, adalimumab, D-penicillamine, oral gold compound, injectable gold compound (intramuscular injection), minocycline, sodium gold thiomalate, auranofin, D-penicillamine, lobenzarit, bucillamine, actarit, cyclophosphamide, azathioprine, methotrexate, mizoribine, cyclosporine, and tacrolimus.

Still other aspects of the disclosure provide for the use of the disclosed antibodies for detecting the presence of CD200R in biological samples. The amount of CD200R detected may be correlated with the expression level of CD200R, which, in turn, is correlated with the activation status of immune cells (e.g., activated T cells, B cells, and monocytes) in the subject.

Medical Use

In another aspect, provided herein is an antibody or an antigen-binding fragment or an immunoconjugate of the present disclosure, a pharmaceutical composition comprising an antibody or an antigen-binding fragment or an immunoconjugate of the present disclosure for use in therapy. Suitably, provided herein is an antibody or an antigen-binding fragment or an immunoconjugate of the present disclosure, or a pharmaceutical composition comprising an antibody or an antigen-binding fragment or immunoconjugate of the present disclosure for use in a method of treatment as disclosed herein.

In another aspect, provided herein is the use of an antibody or an antigen-binding fragment or an immunoconjugate of the present disclosure, or pharmaceutical composition comprising an antibody or an antigen-binding fragment or immunoconjugate of the present disclosure in the manufacture of a medicament for use in therapy, such as for use in a method of treatment as disclosed herein. Disclosed herein is the use of an antibody or antigen-binding fragment thereof in the manufacture of a formulation for the treatment, prevention, amelioration, or reduction of the severity of a CD200R mediated disease. Disclosed herein are antibodies or antigen-binding fragments thereof for use in treating, preventing, ameliorating, or reducing the severity of a CD200R mediated disease. In some embodiments, the CD200R mediated disease is a dermatological disease or condition. In some embodiments, the dermatological disease or condition is selected from atopic dermatitis (AD), contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea. In some embodiments, the dermatological disease or condition is atopic dermatitis (AD). In some embodiments, the dermatological disease or condition is selected from contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea. In some embodiments, the antibody or antigen-binding fragment thereof comprises a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 70, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some embodiments, the HCVR comprises an amino acid sequence having at least 90%, 95%, 96%, 97%, 98%, 99%, or 100% sequence identity to the amino acid sequence as set forth in SEQ ID NO: 72. In some embodiments, the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 72, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications. In some embodiments, the antibody or antigen-binding fragment thereof comprises a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 5, respectively, each with 0 to 3 amino acid modifications, such as 0, 1, 2, or 3 modifications; and (b) the LCVR that comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications. In some embodiments, the HCVR comprises an amino acid sequence having at least 90%, 95%, 96%, 97%, 98%, 99%, or 100% sequence identity to the amino acid sequence as set forth in SEQ ID NO: 71. In some embodiments, the LCVR comprises an amino acid sequence having at least 90%, 95%, 96%, 97%, 98%, 99%, or 100% sequence identity to the amino acid sequence as set forth in SEQ ID NO: 65. In some embodiments, the HCVR comprises an amino acid sequence as set forth in SEQ ID NO: 71, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications. In some embodiments, the antibody or antigen-binding fragment thereof comprises a heavy chain variable region (HCVR) and a light chain variable region (LCVR), wherein: (a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 92, 41, and 69, respectively, each with 0 to 3 amino acid modifications, such as 0, 1, 2, or 3 modifications; and (b) the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 87, and 91, respectively, each with 0 to 3 amino acid modifications, such as 0, 1, 2, or 3 modifications. In some embodiments, X at position 1 of SEQ ID NO: 69 is M. In some embodiments, X at position 1 of SEQ ID NO: 69 is G. In some embodiments, X at position 1 of SEQ ID NO: 87 is G. In some embodiments, X at position 1 of SEQ ID NO: 87 is L. In some embodiments, X at position 2 of SEQ ID NO: 87 is A. In some embodiments, X at position 2 of SEQ ID NO: 87 is G. In some embodiments, X at position 3 of SEQ ID NO: 87 is S. In some embodiments, X at position 3 of SEQ ID NO: 87 is V. In some embodiments, X at position 7 of SEQ ID NO: 87 is D. In some embodiments, X at position 7 of SEQ ID NO: 87 is S. In some embodiments, X at position 7 of SEQ ID NO: 87 is T. In some embodiments, X at position 8 of SEQ ID NO: 91 is W. In some embodiments, X at position 8 of SEQ ID NO: 91 is F. In some embodiments, X at position 3 of SEQ ID NO: 92 is W. In some embodiments, X at position 3 of SEQ ID NO: 92 is F. In some embodiments, the HCVR comprises an amino acid sequence having at least 90%, 95%, 96%, 97%, 98%, 99%, or 100% sequence identity to the amino acid sequence as set forth in SEQ ID NO: 93. In some embodiments, the LCVR comprises an amino acid sequence having at least 90%, 95%, 96%, 97%, 98%, 99%, or 100% sequence identity to the amino acid sequence as set forth in SEQ ID NO: 94. In some embodiments, the HCVR comprises an amino acid sequence as set forth in SEQ ID NO: 93, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 94, with from 0 to 10 amino acid modifications, such as 1, 2, 3, 4, 5, 6, 7, 8, 9 or 10 amino acid modifications. In some embodiments, X at position 1 of SEQ ID NO: 93 is D. In some embodiments, X at position 1 of SEQ ID NO: 93 is E. In some embodiments, X at position 33 of SEQ ID NO: 93 is W. In some embodiments, X at position 33 of SEQ ID NO: 93 is F. In some embodiments, X at position 99 of SEQ ID NO: 93 is M. In some embodiments, X at position 99 of SEQ ID NO: 93 is G. In some embodiments, X at position 50 of SEQ ID NO: 94 is G. In some embodiments, X at position 50 of SEQ ID NO: 94 is L. In some embodiments, X at position 51 of SEQ ID NO: 94 is A. In some embodiments, X at position 51 of SEQ ID NO: 94 is G. In some embodiments, X at position 52 of SEQ ID NO: 94 is S. In some embodiments, X at position 52 of SEQ ID NO: 94 is V. In some embodiments, X at position 56 of SEQ ID NO: 94 is D. In some embodiments, X at position 56 of SEQ ID NO: 94 is S. In some embodiments, X at position 56 of SEQ ID NO: 94 is T. In some embodiments, X at position 96 of SEQ ID NO: 94 is W. In some embodiments, X at position 96 of SEQ ID NO: 94 is F. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 4, 5, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 7, 8, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 1. In some embodiments, the LCVR comprises an amino acid sequence having at least 90%, 95%, 96%, 97%, 98%, 99%, or 100% sequence identity to the amino acid sequence as set forth in SEQ ID NO: 2. In some embodiments, the HCVR comprises an amino acid sequence as set forth in SEQ ID NO: 1, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 2, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 11, 12, 13, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 14, 15, 16, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 9. In some embodiments, the LCVR comprises an amino acid sequence having at least 90%, 95%, 96%, 97%, 98%, 99%, or 100% sequence identity to the amino acid sequence as set forth in SEQ ID NO: 10. In some embodiments, the HCVR comprises an amino acid sequence as set forth in SEQ ID NO: 9, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 10, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 19, 20, or 21, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 22, 23, or 24, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 17. In some embodiments, the LCVR comprises an amino acid sequence having at least 90%, 95%, 96%, 97%, 98%, 99%, or 100% sequence identity to the amino acid sequence as set forth in SEQ ID NO: 18. In some embodiments, the HCVR comprises an amino acid sequence as set forth in SEQ ID NO: 17, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 18, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 27, 28, or 29, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 30, 31, or 32, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 25. In some embodiments, the LCVR comprises an amino acid sequence having at least 90%, 95%, 96%, 97%, 98%, 99%, or 100% sequence identity to the amino acid sequence as set forth in SEQ ID NO: 26. In some embodiments, the HCVR comprises an amino acid sequence as set forth in SEQ ID NO: 25, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 26, with from 0 to 10 amino acid modifications, such as 1, 2, 3, 4, 5, 6, 7, 8, 9 or 10 amino acid modifications. In some embodiments, the antibody or antigen-binding fragment thereof comprises (a) a heavy chain that comprises a heavy chain variable region (HCVR), wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 35, 36, or 37, respectively, with from 0 to 3 amino acid modifications; and (b) a light chain that comprises a light chain variable region (LCVR), wherein the LCVR comprises light chain complementarity determining region 1 (CDRL1), CDRL2, and CDRL3, and wherein CDRL1, CDRL2, and CDRL3 comprise the sequence as set forth in SEQ ID NOs: 38, 39, or 40, respectively, with from 0 to 3 amino acid modifications. In some embodiments, the HCVR comprises an amino acid sequence having at least 90%, 95%, 96%, 97%, 98%, 99%, or 100% sequence identity to the amino acid sequence as set forth in SEQ ID NO: 33. In some embodiments, the LCVR comprises an amino acid sequence having at least 90%, 95%, 96%, 97%, 98%, 99%, or 100% sequence identity to the amino acid sequence as set forth in SEQ ID NO: 34. In some embodiments, the HCVR comprises an amino acid sequence as set forth in SEQ ID NO: 33, with from 0 to 10 amino acid modifications, such as 1, 2, 3, 4, 5, 6, 7, 8, 9 or 10 amino acid modifications. In some embodiments, the LCVR comprises an amino acid sequence as set forth in SEQ ID NO: 34, with from 0 to 10 amino acid modifications, such as 1, 2, 3, 4, 5, 6, 7, 8, 9 or 10 amino acid modifications. In some embodiments, the LCVR is linked to a light chain constant region comprising an amino acid sequence having at least 90%, 95%, 96%, 97%, 98%, 99%, or 100% sequence identity to the amino acid sequence as set forth in SEQ ID NO: 62. In some embodiments, the light chain variable region is linked to a light chain constant region comprising an amino acid sequence as set forth in SEQ ID NO: 62, with from 0 to 10 amino acid modifications, such as 1, 2, 3, 4, 5, 6, 7, 8, 9 or 10 amino acid modifications. In some embodiments, the light chain variable region is linked to a light chain constant region comprising an amino acid sequence having at least 90%, 95%, 96%, 97%, 98%, 99%, or 100% sequence identity to the amino acid sequence as set forth in SEQ ID NO: 64. In some embodiments, the light chain variable region is linked to a light chain constant region comprising an amino acid sequence as set forth in SEQ ID NO: 64, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 61. In some embodiments, the HCVR is linked to a heavy chain constant region comprising an amino acid sequence as set forth in SEQ ID NO: 61, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 63. In some embodiments, the HCVR is linked to a heavy chain constant region comprising an amino acid sequence as set forth in SEQ ID NO: 63, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 75. In some embodiments, the HCVR is linked to a heavy chain constant region comprising an amino acid sequence as set forth in SEQ ID NO: 75, with from 0 to 10 amino acid modifications, such as 1, 2, 3, 4, 5, 6, 7, 8, 9 or 10 amino acid modifications. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 76. In some embodiments, the HCVR is linked to a heavy chain constant region comprising an amino acid sequence as set forth in SEQ ID NO: 76, with from 0 to 10 amino acid modifications, such as 1, 2, 3, 4, 5, 6, 7, 8, 9 or 10 amino acid modifications. In some embodiments, the HCVR is linked to a heavy chain constant region, wherein the heavy chain constant region comprises an Fc region that comprises one or more of the following amino acids: alanine (A) at position 234, alanine (A) at position 235, aspartic acid (D) at position 236, aspartic acid (D) at position 237, aspartic acid (D) at position 238, alanine (A) at position 265, glutamic acid (E) at position 267, glycine (G) at position 271, arginine (R) at position 330, alanine (A) at position 332, or alanine (A) at position 297 (numbering according to EU Index). In some embodiments, the Fc region comprises an aspartic acid (D) at position 238 (EU Index). In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 77. In some embodiments, the HCVR is linked to a heavy chain constant region comprising an amino acid sequence as set forth in SEQ ID NO: 77, with from 0 to 10 amino acid modifications, such as 1, 2, 3, 4, 5, 6, 7, 8, 9 or 10 amino acid modifications. In some embodiments, X at position 121 of SEQ ID NO: 77 is D. In some embodiments, X at position 121 of SEQ ID NO: 77 is P. In some embodiments, X at position 329 of SEQ ID NO: 77 is G. In some embodiments, X at position 329 of SEQ ID NO: 77 is absent. In some embodiments, X at position 330 of SEQ ID NO: 77 is K. In some embodiments, X at position 330 of SEQ ID NO: 77 is absent. In some embodiments, the HCVR is linked to a heavy chain constant region comprising an amino acid sequence having at least 90%, 95%, 96%, 97%, 98%, 99%, or 100% sequence identity to the amino acid sequence as set forth in SEQ ID NO: 78. In some embodiments, the HCVR is linked to a heavy chain constant region comprising an amino acid sequence as set forth in SEQ ID NO: 78, with from 0 to 10 amino acid modifications, such as 1, 2, 3, 4, 5, 6, 7, 8, 9 or 10 amino acid modifications. In some embodiments, X at position 108 of SEQ ID NO: 78 is S. In some embodiments, X at position 108 of SEQ ID NO: 78 is P. In some embodiments, X at position 326 of SEQ ID NO: 78 is G. In some embodiments, X at position 326 of SEQ ID NO: 78 is absent. In some embodiments, X at position 327 of SEQ ID NO: 78 is K. In some embodiments, X at position 327 of SEQ ID NO: 78 is absent. In some embodiments, the method or use further comprises co-administering with one or more agents useful for the treatment and/or prophylaxis of a dermatologic condition, such as atopic dermatitis (AD). Non-limiting examples of such agents include topical corticosteroids (TCS) (e.g., desonid, hydrocortisone, fluocinolone, triamcinolone, betamethasone diproprionate), topical calcineurin inhibitors (TCI) (e.g., tacrolimus, pimecrolimus), topical antimicrobials and antiseptics, cyclosporine, methotrexate, mycophenolate mofetil, interferon gamma, phosphodiesterase 4 (PDE4) inhibitor such as crisaborole, JAK inhibitor (e.g., ruxolitinib, upadacitinib, abrocitinib), systemic glucocorticoids (e.g., prednisone), dupilumab, and anti-IL-13 antibody (e.g., tralokinumab).

An antibody or an antigen-binding fragment thereof that specifically binds CD200R, comprising (a) a heavy chain, wherein the heavy chain comprises a heavy chain variable region (HCVR) of SEQ ID NO: 72 and (b) a light chain, wherein the light chain comprises a light chain variable region (LCVR) of SEQ ID NO: 65, for use in treating an inflammatory disease or condition. In some embodiments, the inflammatory disease or condition is selected from a rheumatological disease or condition, gastrointestinal disease or condition, pulmonary disease or condition, hepatological disease or condition, nephrological disease or condition, and dermatological condition. In some embodiments, the rheumatological condition or disease is selected from rheumatoid arthritis (RA), systemic lupus erythematosus (SLE), lupus nephritis (LN), osteoarthritis (OA), Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, Takayasu arteritis, cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the rheumatological condition or disease is selected from rheumatoid arthritis (RA), systemic lupus erythematosus (SLE), lupus nephritis (LN), and osteoarthritis (OA). In some embodiments, the rheumatological condition or disease is selected Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, and Takayasu arteritis. In some embodiments, the rheumatological condition or disease is selected cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the gastrointestinal disease or condition is selected from ulcerative colitis (UC), Crohn's disease (CD), eosinophilic gastrointestinal disorders (EGIDs), microscopic colitis, ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the gastrointestinal disease or condition is selected from ulcerative colitis (UC) and Crohn's disease (CD). In some embodiments, the gastrointestinal disease or condition is selected from eosinophilic gastrointestinal disorders (EGIDs) and microscopic colitis. In some embodiments, the EGID is selected from eosinophilic esophagitis (EoE), eosinophilic gastroenteritis, and eosinophilic colitis. In some embodiments, the microscopic colitis is selected from collagenous colitis and lymphocytic colitis. In some embodiments, the gastrointestinal disease is selected from ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the pulmonary disease or condition is selected from idiopathic pulmonary fibrosis (IPF), interstitial lung disease (ILD), acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the pulmonary disease or condition is selected from idiopathic pulmonary fibrosis (IPF) and interstitial lung disease (ILD). In some embodiments, the pulmonary disease or condition is selected from acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the hepatological disease or condition is selected from non-alcoholic steatohepatitis (NASH), primary sclerosing cholangitis (PSC), primary biliary cirrhosis (PBC), autoimmune hepatitis, alcoholic steatohepatitis (ASH), alcoholic hepatitis, chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the hepatological disease or condition is non-alcoholic steatohepatitis (NASH). In some embodiments, the hepatological disease or condition is selected from primary sclerosing cholangitis (PSC), primary biliary cirrhosis (PBC), autoimmune hepatitis, alcoholic steatohepatitis (ASH), and alcoholic hepatitis. In some embodiments, the hepatological disease or condition is selected from chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the nephrological disease or condition is selected from diabetic kidney disease (DKD) (diabetic nephropathy), chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the nephrological disease or condition is diabetic kidney disease (DKD) (diabetic nephropathy). In some embodiments, the nephrological disease or condition is selected from chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the dermatological disease or condition is selected from atopic dermatitis (AD), contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea. In some embodiments, the dermatological disease or condition is atopic dermatitis (AD). In some embodiments, the dermatological disease or condition is selected from contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea.

An antibody or an antigen-binding fragment thereof that specifically binds CD200R, comprising (a) a heavy chain, wherein the heavy chain comprises a heavy chain variable region (HCVR) of SEQ ID NO: 71 and (b) a light chain, wherein the light chain comprises a light chain variable region (LCVR) of SEQ ID NO: 65, for use in treating an inflammatory disease or condition. In some embodiments, the inflammatory disease or condition is selected from a rheumatological disease or condition, gastrointestinal disease or condition, pulmonary disease or condition, hepatological disease or condition, nephrological disease or condition, and dermatological condition. In some embodiments, the rheumatological condition or disease is selected from rheumatoid arthritis (RA), systemic lupus erythematosus (SLE), lupus nephritis (LN), osteoarthritis (OA), Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, Takayasu arteritis, cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the rheumatological condition or disease is selected from rheumatoid arthritis (RA), systemic lupus erythematosus (SLE), lupus nephritis (LN), and osteoarthritis (OA). In some embodiments, the rheumatological condition or disease is selected Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, and Takayasu arteritis. In some embodiments, the rheumatological condition or disease is selected cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the gastrointestinal disease or condition is selected from ulcerative colitis (UC), Crohn's disease (CD), eosinophilic gastrointestinal disorders (EGIDs), microscopic colitis, ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the gastrointestinal disease or condition is selected from ulcerative colitis (UC) and Crohn's disease (CD). In some embodiments, the gastrointestinal disease or condition is selected from eosinophilic gastrointestinal disorders (EGIDs) and microscopic colitis. In some embodiments, the EGID is selected from eosinophilic esophagitis (EoE), eosinophilic gastroenteritis, and eosinophilic colitis. In some embodiments, the microscopic colitis is selected from collagenous colitis and lymphocytic colitis. In some embodiments, the gastrointestinal disease is selected from ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the pulmonary disease or condition is selected from idiopathic pulmonary fibrosis (IPF), interstitial lung disease (ILD), acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the pulmonary disease or condition is selected from idiopathic pulmonary fibrosis (IPF) and interstitial lung disease (ILD). In some embodiments, the pulmonary disease or condition is selected from acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the hepatological disease or condition is selected from non-alcoholic steatohepatitis (NASH), primary sclerosing cholangitis (PSC), primary biliary cirrhosis (PBC), autoimmune hepatitis, alcoholic steatohepatitis (ASH), alcoholic hepatitis, chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the hepatological disease or condition is non-alcoholic steatohepatitis (NASH). In some embodiments, the hepatological disease or condition is selected from primary sclerosing cholangitis (PSC), primary biliary cirrhosis (PBC), autoimmune hepatitis, alcoholic steatohepatitis (ASH), and alcoholic hepatitis. In some embodiments, the hepatological disease or condition is selected from chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the nephrological disease or condition is selected from diabetic kidney disease (DKD) (diabetic nephropathy), chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the nephrological disease or condition is diabetic kidney disease (DKD) (diabetic nephropathy). In some embodiments, the nephrological disease or condition is selected from chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the dermatological disease or condition is selected from atopic dermatitis (AD), contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea. In some embodiments, the dermatological disease or condition is atopic dermatitis (AD). In some embodiments, the dermatological disease or condition is selected from contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea.

An antibody or an antigen-binding fragment thereof that specifically binds CD200R, comprising (a) a heavy chain, wherein the heavy chain comprises a heavy chain variable region (HCVR) of SEQ ID NO: 93 and (b) a light chain, wherein the light chain comprises a light chain variable region (LCVR) of SEQ ID NO: 94, for use in treating an inflammatory disease or condition. In some embodiments, X at position 1 of SEQ ID NO: 93 is D. In some embodiments, X at position 1 of SEQ ID NO: 93 is E. In some embodiments, X at position 33 of SEQ ID NO: 93 is W. In some embodiments, X at position 33 of SEQ ID NO: 93 is F. In some embodiments, X at position 99 of SEQ ID NO: 93 is M. In some embodiments, X at position 99 of SEQ ID NO: 93 is G. In some embodiments, X at position 50 of SEQ ID NO: 94 is G. In some embodiments, X at position 50 of SEQ ID NO: 94 is L. In some embodiments, X at position 51 of SEQ ID NO: 94 is A. In some embodiments, X at position 51 of SEQ ID NO: 94 is G. In some embodiments, X at position 52 of SEQ ID NO: 94 is S. In some embodiments, X at position 52 of SEQ ID NO: 94 is V. In some embodiments, X at position 56 of SEQ ID NO: 94 is D. In some embodiments, X at position 56 of SEQ ID NO: 94 is S. In some embodiments, X at position 56 of SEQ ID NO: 94 is T. In some embodiments, X at position 96 of SEQ ID NO: 94 is W. In some embodiments, X at position 96 of SEQ ID NO: 94 is F. In some embodiments, the inflammatory disease or condition is selected from a rheumatological disease or condition, gastrointestinal disease or condition, pulmonary disease or condition, hepatological disease or condition, nephrological disease or condition, and dermatological condition. In some embodiments, the rheumatological condition or disease is selected from rheumatoid arthritis (RA), systemic lupus erythematosus (SLE), lupus nephritis (LN), osteoarthritis (OA), Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, Takayasu arteritis, cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the rheumatological condition or disease is selected from rheumatoid arthritis (RA), systemic lupus erythematosus (SLE), lupus nephritis (LN), and osteoarthritis (OA). In some embodiments, the rheumatological condition or disease is selected Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, and Takayasu arteritis. In some embodiments, the rheumatological condition or disease is selected cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the gastrointestinal disease or condition is selected from ulcerative colitis (UC), Crohn's disease (CD), eosinophilic gastrointestinal disorders (EGIDs), microscopic colitis, ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the gastrointestinal disease or condition is selected from ulcerative colitis (UC) and Crohn's disease (CD). In some embodiments, the gastrointestinal disease or condition is selected from eosinophilic gastrointestinal disorders (EGIDs) and microscopic colitis. In some embodiments, the EGID is selected from eosinophilic esophagitis (EoE), eosinophilic gastroenteritis, and eosinophilic colitis. In some embodiments, the microscopic colitis is selected from collagenous colitis and lymphocytic colitis. In some embodiments, the gastrointestinal disease is selected from ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the pulmonary disease or condition is selected from idiopathic pulmonary fibrosis (IPF), interstitial lung disease (ILD), acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the pulmonary disease or condition is selected from idiopathic pulmonary fibrosis (IPF) and interstitial lung disease (ILD). In some embodiments, the pulmonary disease or condition is selected from acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the hepatological disease or condition is selected from non-alcoholic steatohepatitis (NASH), primary sclerosing cholangitis (PSC), primary biliary cirrhosis (PBC), autoimmune hepatitis, alcoholic steatohepatitis (ASH), alcoholic hepatitis, chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the hepatological disease or condition is non-alcoholic steatohepatitis (NASH). In some embodiments, the hepatological disease or condition is selected from primary sclerosing cholangitis (PSC), primary biliary cirrhosis (PBC), autoimmune hepatitis, alcoholic steatohepatitis (ASH), and alcoholic hepatitis. In some embodiments, the hepatological disease or condition is selected from chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the nephrological disease or condition is selected from diabetic kidney disease (DKD) (diabetic nephropathy), chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the nephrological disease or condition is diabetic kidney disease (DKD) (diabetic nephropathy). In some embodiments, the nephrological disease or condition is selected from chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the dermatological disease or condition is selected from atopic dermatitis (AD), contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea. In some embodiments, the dermatological disease or condition is atopic dermatitis (AD). In some embodiments, the dermatological disease or condition is selected from contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea.

An antibody or an antigen-binding fragment thereof that specifically binds CD200R, comprising (a) a heavy chain of SEQ ID NO: 82 and (b) a light chain of SEQ ID NO: 86, for use in treating an inflammatory disease or condition. In some embodiments, the inflammatory disease or condition is selected from a rheumatological disease or condition, gastrointestinal disease or condition, pulmonary disease or condition, hepatological disease or condition, nephrological disease or condition, and dermatological condition. In some embodiments, the rheumatological condition or disease is selected from rheumatoid arthritis (RA), systemic lupus erythematosus (SLE), lupus nephritis (LN), osteoarthritis (OA), Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, Takayasu arteritis, cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the rheumatological condition or disease is selected from rheumatoid arthritis (RA), systemic lupus erythematosus (SLE), lupus nephritis (LN), and osteoarthritis (OA). In some embodiments, the rheumatological condition or disease is selected Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, and Takayasu arteritis. In some embodiments, the rheumatological condition or disease is selected cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the gastrointestinal disease or condition is selected from ulcerative colitis (UC), Crohn's disease (CD), eosinophilic gastrointestinal disorders (EGIDs), microscopic colitis, ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the gastrointestinal disease or condition is selected from ulcerative colitis (UC) and Crohn's disease (CD). In some embodiments, the gastrointestinal disease or condition is selected from eosinophilic gastrointestinal disorders (EGIDs) and microscopic colitis. In some embodiments, the EGID is selected from eosinophilic esophagitis (EoE), eosinophilic gastroenteritis, and eosinophilic colitis. In some embodiments, the microscopic colitis is selected from collagenous colitis and lymphocytic colitis. In some embodiments, the gastrointestinal disease is selected from ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the pulmonary disease or condition is selected from idiopathic pulmonary fibrosis (IPF), interstitial lung disease (ILD), acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the pulmonary disease or condition is selected from idiopathic pulmonary fibrosis (IPF) and interstitial lung disease (ILD). In some embodiments, the pulmonary disease or condition is selected from acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the hepatological disease or condition is selected from non-alcoholic steatohepatitis (NASH), primary sclerosing cholangitis (PSC), primary biliary cirrhosis (PBC), autoimmune hepatitis, alcoholic steatohepatitis (ASH), alcoholic hepatitis, chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the hepatological disease or condition is non-alcoholic steatohepatitis (NASH). In some embodiments, the hepatological disease or condition is selected from primary sclerosing cholangitis (PSC), primary biliary cirrhosis (PBC), autoimmune hepatitis, alcoholic steatohepatitis (ASH), and alcoholic hepatitis. In some embodiments, the hepatological disease or condition is selected from chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the nephrological disease or condition is selected from diabetic kidney disease (DKD) (diabetic nephropathy), chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the nephrological disease or condition is diabetic kidney disease (DKD) (diabetic nephropathy). In some embodiments, the nephrological disease or condition is selected from chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the dermatological disease or condition is selected from atopic dermatitis (AD), contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea. In some embodiments, the dermatological disease or condition is atopic dermatitis (AD). In some embodiments, the dermatological disease or condition is selected from contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea.

An antibody or an antigen-binding fragment thereof that specifically binds CD200R, comprising (a) a heavy chain of SEQ ID NO: 83 and (b) a light chain of SEQ ID NO: 86, for use in treating an inflammatory disease or condition. In some embodiments, the inflammatory disease or condition is selected from a rheumatological disease or condition, gastrointestinal disease or condition, pulmonary disease or condition, hepatological disease or condition, nephrological disease or condition, and dermatological condition. In some embodiments, the rheumatological condition or disease is selected from rheumatoid arthritis (RA), systemic lupus erythematosus (SLE), lupus nephritis (LN), osteoarthritis (OA), Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, Takayasu arteritis, cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the rheumatological condition or disease is selected from rheumatoid arthritis (RA), systemic lupus erythematosus (SLE), lupus nephritis (LN), and osteoarthritis (OA). In some embodiments, the rheumatological condition or disease is selected Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, and Takayasu arteritis. In some embodiments, the rheumatological condition or disease is selected cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the gastrointestinal disease or condition is selected from ulcerative colitis (UC), Crohn's disease (CD), eosinophilic gastrointestinal disorders (EGIDs), microscopic colitis, ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the gastrointestinal disease or condition is selected from ulcerative colitis (UC) and Crohn's disease (CD). In some embodiments, the gastrointestinal disease or condition is selected from eosinophilic gastrointestinal disorders (EGIDs) and microscopic colitis. In some embodiments, the EGID is selected from eosinophilic esophagitis (EoE), eosinophilic gastroenteritis, and eosinophilic colitis. In some embodiments, the microscopic colitis is selected from collagenous colitis and lymphocytic colitis. In some embodiments, the gastrointestinal disease is selected from ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the pulmonary disease or condition is selected from idiopathic pulmonary fibrosis (IPF), interstitial lung disease (TLD), acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the pulmonary disease or condition is selected from idiopathic pulmonary fibrosis (IPF) and interstitial lung disease (ILD). In some embodiments, the pulmonary disease or condition is selected from acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the hepatological disease or condition is selected from non-alcoholic steatohepatitis (NASH), primary sclerosing cholangitis (PSC), primary biliary cirrhosis (PBC), autoimmune hepatitis, alcoholic steatohepatitis (ASH), alcoholic hepatitis, chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the hepatological disease or condition is non-alcoholic steatohepatitis (NASH). In some embodiments, the hepatological disease or condition is selected from primary sclerosing cholangitis (PSC), primary biliary cirrhosis (PBC), autoimmune hepatitis, alcoholic steatohepatitis (ASH), and alcoholic hepatitis. In some embodiments, the hepatological disease or condition is selected from chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the nephrological disease or condition is selected from diabetic kidney disease (DKD) (diabetic nephropathy), chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the nephrological disease or condition is diabetic kidney disease (DKD) (diabetic nephropathy). In some embodiments, the nephrological disease or condition is selected from chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the dermatological disease or condition is selected from atopic dermatitis (AD), contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea. In some embodiments, the dermatological disease or condition is atopic dermatitis (AD). In some embodiments, the dermatological disease or condition is selected from contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea.

An antibody or an antigen-binding fragment thereof that specifically binds CD200R, comprising (a) a heavy chain of SEQ ID NO: 84 and (b) a light chain of SEQ ID NO: 86, for use in treating an inflammatory disease or condition. In some embodiments, the inflammatory disease or condition is selected from a rheumatological disease or condition, gastrointestinal disease or condition, pulmonary disease or condition, hepatological disease or condition, nephrological disease or condition, and dermatological condition. In some embodiments, the rheumatological condition or disease is selected from rheumatoid arthritis (RA), systemic lupus erythematosus (SLE), lupus nephritis (LN), osteoarthritis (OA), Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, Takayasu arteritis, cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the rheumatological condition or disease is selected from rheumatoid arthritis (RA), systemic lupus erythematosus (SLE), lupus nephritis (LN), and osteoarthritis (OA). In some embodiments, the rheumatological condition or disease is selected Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, and Takayasu arteritis. In some embodiments, the rheumatological condition or disease is selected cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the gastrointestinal disease or condition is selected from ulcerative colitis (UC), Crohn's disease (CD), eosinophilic gastrointestinal disorders (EGIDs), microscopic colitis, ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the gastrointestinal disease or condition is selected from ulcerative colitis (UC) and Crohn's disease (CD). In some embodiments, the gastrointestinal disease or condition is selected from eosinophilic gastrointestinal disorders (EGIDs) and microscopic colitis. In some embodiments, the EGID is selected from eosinophilic esophagitis (EoE), eosinophilic gastroenteritis, and eosinophilic colitis. In some embodiments, the microscopic colitis is selected from collagenous colitis and lymphocytic colitis. In some embodiments, the gastrointestinal disease is selected from ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the pulmonary disease or condition is selected from idiopathic pulmonary fibrosis (IPF), interstitial lung disease (TLD), acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the pulmonary disease or condition is selected from idiopathic pulmonary fibrosis (IPF) and interstitial lung disease (ILD). In some embodiments, the pulmonary disease or condition is selected from acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the hepatological disease or condition is selected from non-alcoholic steatohepatitis (NASH), primary sclerosing cholangitis (PSC), primary biliary cirrhosis (PBC), autoimmune hepatitis, alcoholic steatohepatitis (ASH), alcoholic hepatitis, chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the hepatological disease or condition is non-alcoholic steatohepatitis (NASH). In some embodiments, the hepatological disease or condition is selected from primary sclerosing cholangitis (PSC), primary biliary cirrhosis (PBC), autoimmune hepatitis, alcoholic steatohepatitis (ASH), and alcoholic hepatitis. In some embodiments, the hepatological disease or condition is selected from chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the nephrological disease or condition is selected from diabetic kidney disease (DKD) (diabetic nephropathy), chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the nephrological disease or condition is diabetic kidney disease (DKD) (diabetic nephropathy). In some embodiments, the nephrological disease or condition is selected from chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the dermatological disease or condition is selected from atopic dermatitis (AD), contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea. In some embodiments, the dermatological disease or condition is atopic dermatitis (AD). In some embodiments, the dermatological disease or condition is selected from contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea.

An antibody or an antigen-binding fragment thereof that specifically binds CD200R, comprising (a) a heavy chain of SEQ ID NO: 85 and (b) a light chain of SEQ ID NO: 86, for use in treating an inflammatory disease or condition. In some embodiments, the inflammatory disease or condition is selected from a rheumatological disease or condition, gastrointestinal disease or condition, pulmonary disease or condition, hepatological disease or condition, nephrological disease or condition, and dermatological condition. In some embodiments, the rheumatological condition or disease is selected from rheumatoid arthritis (RA), systemic lupus erythematosus (SLE), lupus nephritis (LN), osteoarthritis (OA), Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, Takayasu arteritis, cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the rheumatological condition or disease is selected from rheumatoid arthritis (RA), systemic lupus erythematosus (SLE), lupus nephritis (LN), and osteoarthritis (OA). In some embodiments, the rheumatological condition or disease is selected Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, and Takayasu arteritis. In some embodiments, the rheumatological condition or disease is selected cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the gastrointestinal disease or condition is selected from ulcerative colitis (UC), Crohn's disease (CD), eosinophilic gastrointestinal disorders (EGIDs), microscopic colitis, ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the gastrointestinal disease or condition is selected from ulcerative colitis (UC) and Crohn's disease (CD). In some embodiments, the gastrointestinal disease or condition is selected from eosinophilic gastrointestinal disorders (EGIDs) and microscopic colitis. In some embodiments, the EGID is selected from eosinophilic esophagitis (EoE), eosinophilic gastroenteritis, and eosinophilic colitis. In some embodiments, the microscopic colitis is selected from collagenous colitis and lymphocytic colitis. In some embodiments, the gastrointestinal disease is selected from ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the pulmonary disease or condition is selected from idiopathic pulmonary fibrosis (IPF), interstitial lung disease (ILD), acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the pulmonary disease or condition is selected from idiopathic pulmonary fibrosis (IPF) and interstitial lung disease (ILD). In some embodiments, the pulmonary disease or condition is selected from acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the hepatological disease or condition is selected from non-alcoholic steatohepatitis (NASH), primary sclerosing cholangitis (PSC), primary biliary cirrhosis (PBC), autoimmune hepatitis, alcoholic steatohepatitis (ASH), alcoholic hepatitis, chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the hepatological disease or condition is non-alcoholic steatohepatitis (NASH). In some embodiments, the hepatological disease or condition is selected from primary sclerosing cholangitis (PSC), primary biliary cirrhosis (PBC), autoimmune hepatitis, alcoholic steatohepatitis (ASH), and alcoholic hepatitis. In some embodiments, the hepatological disease or condition is selected from chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the nephrological disease or condition is selected from diabetic kidney disease (DKD) (diabetic nephropathy), chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the nephrological disease or condition is diabetic kidney disease (DKD) (diabetic nephropathy). In some embodiments, the nephrological disease or condition is selected from chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the dermatological disease or condition is selected from atopic dermatitis (AD), contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea. In some embodiments, the dermatological disease or condition is atopic dermatitis (AD). In some embodiments, the dermatological disease or condition is selected from contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea.

An antibody or an antigen-binding fragment thereof that specifically binds CD200R, comprising (a) a heavy chain of SEQ ID NO: 95 and (b) a light chain of SEQ ID NO: 98, for use in treating an inflammatory disease or condition. In some embodiments, X at position 1 of SEQ ID NO: 95 is D. In some embodiments, X at position 1 of SEQ ID NO: 95 is E. In some embodiments, X at position 33 of SEQ ID NO: 95 is W. In some embodiments, X at position 33 of SEQ ID NO: 95 is F. In some embodiments, X at position 99 of SEQ ID NO: 95 is M. In some embodiments, X at position 99 of SEQ ID NO: 95 is G. In some embodiments, X at position 235 of SEQ ID NO: 95 is D. In some embodiments, X at position 235 of SEQ ID NO: 95 is P. In some embodiments, X at position 443 of SEQ ID NO: 95 is G. In some embodiments, X at positions 443 and 444 of SEQ ID NO: 95 are absent. In some embodiments, X at position 444 is SEQ ID NO: 95 is K. In some embodiments, X at position 444 is SEQ ID NO: 95 is absent. In some embodiments, X at position 50 of SEQ ID NO: 98 is G. In some embodiments, X at position 50 of SEQ ID NO: 98 is L. In some embodiments, X at position 51 of SEQ ID NO: 98 is A. In some embodiments, X at position 51 of SEQ ID NO: 98 is G. In some embodiments, X at position 52 of SEQ ID NO: 98 is S. In some embodiments, X at position 52 of SEQ ID NO: 98 V. In some embodiments, X at position 56 of SEQ ID NO: 98 is D. In some embodiments, X at position 56 of SEQ ID NO: 98 is S. In some embodiments, X at position 56 of SEQ ID NO: 98 is T. In some embodiments, X at position 96 of SEQ ID NO: 98 is W. In some embodiments, X at position 96 of SEQ ID NO: 98 is F. In some embodiments, the inflammatory disease or condition is selected from a rheumatological disease or condition, gastrointestinal disease or condition, pulmonary disease or condition, hepatological disease or condition, nephrological disease or condition, and dermatological condition. In some embodiments, the rheumatological condition or disease is selected from rheumatoid arthritis (RA), systemic lupus erythematosus (SLE), lupus nephritis (LN), osteoarthritis (OA), Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, Takayasu arteritis, cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the rheumatological condition or disease is selected from rheumatoid arthritis (RA), systemic lupus erythematosus (SLE), lupus nephritis (LN), and osteoarthritis (OA). In some embodiments, the rheumatological condition or disease is selected Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, and Takayasu arteritis. In some embodiments, the rheumatological condition or disease is selected cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the gastrointestinal disease or condition is selected from ulcerative colitis (UC), Crohn's disease (CD), eosinophilic gastrointestinal disorders (EGIDs), microscopic colitis, ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the gastrointestinal disease or condition is selected from ulcerative colitis (UC) and Crohn's disease (CD). In some embodiments, the gastrointestinal disease or condition is selected from eosinophilic gastrointestinal disorders (EGIDs) and microscopic colitis. In some embodiments, the EGID is selected from eosinophilic esophagitis (EoE), eosinophilic gastroenteritis, and eosinophilic colitis. In some embodiments, the microscopic colitis is selected from collagenous colitis and lymphocytic colitis. In some embodiments, the gastrointestinal disease is selected from ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the pulmonary disease or condition is selected from idiopathic pulmonary fibrosis (IPF), interstitial lung disease (ILD), acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the pulmonary disease or condition is selected from idiopathic pulmonary fibrosis (IPF) and interstitial lung disease (ILD). In some embodiments, the pulmonary disease or condition is selected from acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the hepatological disease or condition is selected from non-alcoholic steatohepatitis (NASH), primary sclerosing cholangitis (PSC), primary biliary cirrhosis (PBC), autoimmune hepatitis, alcoholic steatohepatitis (ASH), alcoholic hepatitis, chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the hepatological disease or condition is non-alcoholic steatohepatitis (NASH). In some embodiments, the hepatological disease or condition is selected from primary sclerosing cholangitis (PSC), primary biliary cirrhosis (PBC), autoimmune hepatitis, alcoholic steatohepatitis (ASH), and alcoholic hepatitis. In some embodiments, the hepatological disease or condition is selected from chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the nephrological disease or condition is selected from diabetic kidney disease (DKD) (diabetic nephropathy), chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the nephrological disease or condition is diabetic kidney disease (DKD) (diabetic nephropathy). In some embodiments, the nephrological disease or condition is selected from chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the dermatological disease or condition is selected from atopic dermatitis (AD), contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea. In some embodiments, the dermatological disease or condition is atopic dermatitis (AD). In some embodiments, the dermatological disease or condition is selected from contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea.

An antibody or an antigen-binding fragment thereof that specifically binds CD200R, comprising (a) a heavy chain of SEQ ID NO: 96 and (b) a light chain of SEQ ID NO: 98, for use in treating an inflammatory disease or condition. In some embodiments, X at position 1 of SEQ ID NO: 96 is D. In some embodiments, X at position 1 of SEQ ID NO: 96 is E. In some embodiments, X at position 33 of SEQ ID NO: 96 is W. In some embodiments, X at position 33 of SEQ ID NO: 96 is F. In some embodiments, X at position 99 of SEQ ID NO: 96 is M. In some embodiments, X at position 99 of SEQ ID NO: 96 is G. In some embodiments, X at position 222 of SEQ ID NO: 96 is S. In some embodiments, X at position 222 of SEQ ID NO: 96 is P. In some embodiments, X at position 440 of SEQ ID NO: 96 is G. In some embodiments, X at positions 440 and 441 of SEQ ID NO: 96 are absent. In some embodiments, X at position 441 of SEQ ID NO: 96 is K. In some embodiments, X at position 441 of SEQ ID NO: 96 is absent. In some embodiments, X at position 50 of SEQ ID NO: 98 is G. In some embodiments, X at position 50 of SEQ ID NO: 98 is L. In some embodiments, X at position 51 of SEQ ID NO: 98 is A. In some embodiments, X at position 51 of SEQ ID NO: 98 is G. In some embodiments, X at position 52 of SEQ ID NO: 98 is S. In some embodiments, X at position 52 of SEQ ID NO: 98 V. In some embodiments, X at position 56 of SEQ ID NO: 98 is D. In some embodiments, X at position 56 of SEQ ID NO: 98 is S. In some embodiments, X at position 56 of SEQ ID NO: 98 is T. In some embodiments, X at position 96 of SEQ ID NO: 98 is W. In some embodiments, X at position 96 of SEQ ID NO: 98 is F. In some embodiments, the inflammatory disease or condition is selected from a rheumatological disease or condition, gastrointestinal disease or condition, pulmonary disease or condition, hepatological disease or condition, nephrological disease or condition, and dermatological condition. In some embodiments, the rheumatological condition or disease is selected from rheumatoid arthritis (RA), systemic lupus erythematosus (SLE), lupus nephritis (LN), osteoarthritis (OA), Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, Takayasu arteritis, cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the rheumatological condition or disease is selected from rheumatoid arthritis (RA), systemic lupus erythematosus (SLE), lupus nephritis (LN), and osteoarthritis (OA). In some embodiments, the rheumatological condition or disease is selected Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, and Takayasu arteritis. In some embodiments, the rheumatological condition or disease is selected cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the gastrointestinal disease or condition is selected from ulcerative colitis (UC), Crohn's disease (CD), eosinophilic gastrointestinal disorders (EGIDs), microscopic colitis, ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the gastrointestinal disease or condition is selected from ulcerative colitis (UC) and Crohn's disease (CD). In some embodiments, the gastrointestinal disease or condition is selected from eosinophilic gastrointestinal disorders (EGIDs) and microscopic colitis. In some embodiments, the EGID is selected from eosinophilic esophagitis (EoE), eosinophilic gastroenteritis, and eosinophilic colitis. In some embodiments, the microscopic colitis is selected from collagenous colitis and lymphocytic colitis. In some embodiments, the gastrointestinal disease is selected from ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the pulmonary disease or condition is selected from idiopathic pulmonary fibrosis (IPF), interstitial lung disease (ILD), acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the pulmonary disease or condition is selected from idiopathic pulmonary fibrosis (IPF) and interstitial lung disease (ILD). In some embodiments, the pulmonary disease or condition is selected from acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the hepatological disease or condition is selected from non-alcoholic steatohepatitis (NASH), primary sclerosing cholangitis (PSC), primary biliary cirrhosis (PBC), autoimmune hepatitis, alcoholic steatohepatitis (ASH), alcoholic hepatitis, chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the hepatological disease or condition is non-alcoholic steatohepatitis (NASH). In some embodiments, the hepatological disease or condition is selected from primary sclerosing cholangitis (PSC), primary biliary cirrhosis (PBC), autoimmune hepatitis, alcoholic steatohepatitis (ASH), and alcoholic hepatitis. In some embodiments, the hepatological disease or condition is selected from chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the nephrological disease or condition is selected from diabetic kidney disease (DKD) (diabetic nephropathy), chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the nephrological disease or condition is diabetic kidney disease (DKD) (diabetic nephropathy). In some embodiments, the nephrological disease or condition is selected from chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the dermatological disease or condition is selected from atopic dermatitis (AD), contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea. In some embodiments, the dermatological disease or condition is atopic dermatitis (AD). In some embodiments, the dermatological disease or condition is selected from contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea.

An antibody or an antigen-binding fragment thereof that specifically binds CD200R, comprising (a) a heavy chain of SEQ ID NO: 97 and (b) a light chain of SEQ ID NO: 98, for use in treating an inflammatory disease or condition. In some embodiments, X at position 1 of SEQ ID NO: 97 is D. In some embodiments, X at position 1 of SEQ ID NO: 97 is E. In some embodiments, X at position 33 of SEQ ID NO: 97 is W. In some embodiments, X at position 33 of SEQ ID NO: 97 is F. In some embodiments, X at position 99 of SEQ ID NO: 97 is M. In some embodiments, X at position 99 of SEQ ID NO: 97 is G. In some embodiments, X at position 437 of SEQ ID NO: 97 is G. In some embodiments, X at position 437 and 438 of SEQ ID NO: 97 are absent. In some embodiments, X at position 438 of SEQ ID NO: 97 is K. In some embodiments, X at position 438 of SEQ ID NO: 97 is absent. In some embodiments, X at position 50 of SEQ ID NO: 98 is G. In some embodiments, X at position 50 of SEQ ID NO: 98 is L. In some embodiments, X at position 51 of SEQ ID NO: 98 is A. In some embodiments, X at position 51 of SEQ ID NO: 98 is G. In some embodiments, X at position 52 of SEQ ID NO: 98 is S. In some embodiments, X at position 52 of SEQ ID NO: 98 V. In some embodiments, X at position 56 of SEQ ID NO: 98 is D. In some embodiments, X at position 56 of SEQ ID NO: 98 is S. In some embodiments, X at position 56 of SEQ ID NO: 98 is T. In some embodiments, X at position 96 of SEQ ID NO: 98 is W. In some embodiments, X at position 96 of SEQ ID NO: 98 is F. In some embodiments, the inflammatory disease or condition is selected from a rheumatological disease or condition, gastrointestinal disease or condition, pulmonary disease or condition, hepatological disease or condition, nephrological disease or condition, and dermatological condition. In some embodiments, the rheumatological condition or disease is selected from rheumatoid arthritis (RA), systemic lupus erythematosus (SLE), lupus nephritis (LN), osteoarthritis (OA), Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, Takayasu arteritis, cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the rheumatological condition or disease is selected from rheumatoid arthritis (RA), systemic lupus erythematosus (SLE), lupus nephritis (LN), and osteoarthritis (OA). In some embodiments, the rheumatological condition or disease is selected Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, and Takayasu arteritis. In some embodiments, the rheumatological condition or disease is selected cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA. In some embodiments, the gastrointestinal disease or condition is selected from ulcerative colitis (UC), Crohn's disease (CD), eosinophilic gastrointestinal disorders (EGIDs), microscopic colitis, ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the gastrointestinal disease or condition is selected from ulcerative colitis (UC) and Crohn's disease (CD). In some embodiments, the gastrointestinal disease or condition is selected from eosinophilic gastrointestinal disorders (EGIDs) and microscopic colitis. In some embodiments, the EGID is selected from eosinophilic esophagitis (EoE), eosinophilic gastroenteritis, and eosinophilic colitis. In some embodiments, the microscopic colitis is selected from collagenous colitis and lymphocytic colitis. In some embodiments, the gastrointestinal disease is selected from ulcerative proctitis, proctosignmoiditis, left-sided colitis, extensive colitis, pancolitis, ileocolitis, ileitis, gastroduodenal CD, jejunoileitis, and Crohn's (granulomatous) colitis. In some embodiments, the pulmonary disease or condition is selected from idiopathic pulmonary fibrosis (IPF), interstitial lung disease (TLD), acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the pulmonary disease or condition is selected from idiopathic pulmonary fibrosis (IPF) and interstitial lung disease (ILD). In some embodiments, the pulmonary disease or condition is selected from acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis. In some embodiments, the hepatological disease or condition is selected from non-alcoholic steatohepatitis (NASH), primary sclerosing cholangitis (PSC), primary biliary cirrhosis (PBC), autoimmune hepatitis, alcoholic steatohepatitis (ASH), alcoholic hepatitis, chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the hepatological disease or condition is non-alcoholic steatohepatitis (NASH). In some embodiments, the hepatological disease or condition is selected from primary sclerosing cholangitis (PSC), primary biliary cirrhosis (PBC), autoimmune hepatitis, alcoholic steatohepatitis (ASH), and alcoholic hepatitis. In some embodiments, the hepatological disease or condition is selected from chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus. In some embodiments, the nephrological disease or condition is selected from diabetic kidney disease (DKD) (diabetic nephropathy), chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the nephrological disease or condition is diabetic kidney disease (DKD) (diabetic nephropathy). In some embodiments, the nephrological disease or condition is selected from chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia. In some embodiments, the dermatological disease or condition is selected from atopic dermatitis (AD), contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea. In some embodiments, the dermatological disease or condition is atopic dermatitis (AD). In some embodiments, the dermatological disease or condition is selected from contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea.

Therapeutic Agents

In some embodiments, any of the kits, or compositions disclosed herein further comprise one or more additional therapeutic agents. In some embodiments, any of the methods or uses disclosed herein further comprise co-administering one or more additional therapeutic agents. In some embodiments, an additional therapeutic agent includes one or more of 2,4-dinitrophenol, 25HC3S, A-4250, A-4368, A-717, AAT-IV, AAV8-FGF19 variant M70, abatacept, AB-1711, ABBV-022, ABBV-105, ABBV-154, ABBV-257, abrocitinib, ABT-122, ABT-494, ABX-464, AC-0058, AC-3174, acalabrutinib, ACE-1334, aceclofenac, acetylsalicylic acid (aspirin), ACF-TEI, acitretin, actarit, ACQT-1127, adalimumab, adipocell, AdMSCs, ADP-12778, ADSTEM, ADX-914, AER-601, AFB-035 (izokibep), afimetoran (BMS-986256), aganirsen, AGN-242266, AJ-303, AK-101, AK-106, AK-20, AK3280, AKP-11, AL-24-2A1, alanyl-glutamine, albiglutide, ALD-R491, aldesleukin, ALE-F02, alefacept, Alequel, alicaforsen, alipogene tiparvovec, Alitretinoin, ALLN-346, ALLO-ASC-CD, Allocetra, allogeneic mesenchymal stem cell therapy, ALPN-101, ALPN-303, ALT-801, ALV-304, ALX-0061, AM-01, AMG-133, AMG-592, AMG-609, AMG-966, Amilo-SMER, aminopterin, amiselimod, AMP-activated protein kinase inhibitors, Ampion, AMT-101, AMT-126, AMX-342, AN-3015, anakinra, anandamide, Anapsos, ANB-030, ANB-032, anchorins, ANG-3298, anifrolumab, annexin V-128, annexuzlimab, anti-CXCR3 mAb, anti-IL-4/IL-13 antibodies, anti-IL-1/anti-TNF antibodies, anti-IL-4/IL-13 antibodies, anti IL1RAP antibodies, anti-iRhom2 monoclonal antibodies, anti-SCF248 antibodies, anti-SCF248 monoclonal antibodies, anti-TAGE antibodies, anti-TAGE monoclonal antibodies, anti-TGF beta antibodies, AP-005, AP-1189, APL-1401, apremilast, aramchol, ARD-101, ARG-301, ARI-3037MO, ARN-4079, ARQ-250, arsenic trioxide, artenimol, ASD-003, ASKP-1240, ASLAN-003, ASP-1002, ASP-1617, ASP-3291, ASP-5094, ASP-8232, AST-005, AST-120, AT-132, atacicept, ATB-1606, ATI-2138, ATIR-101, atorvastatin, auranofin, avexitide, AVID-200, AVO-101, AVTX-002 (AEVI-002), AVX-001, AVX-470, AX-1505, azathioprine, AZD-058, AZD-9567, azemiglitazone potassium, azm-198B-244, B-1344, B-1654, BBT-401, balcinrenone, balsalazide, baricitinib, basiliximab, BAY-1830839, BAY-2327949, BEBT-305, BEBT-503, beclomethasone dipropionate, begelomab, belapectin, belatacept, belimumab, BEN-2293, benaglutide, bendamustine hydrochloride, bermekimab, bertilimumab, Betaine anhydrous, betamethasone, BI-1015550, BI-1467335, BI-456906, BI-655064 (ABBV-323), BI-685509, BI-705564, BI-730357, BI-730460, BIIB-059, bimekizumab, BIO-300, BiP (rheumatoid arthritis), BIVV-009, BLHP-006, blisibimod, BMC-321, BMC-322, BML-258, BMS-986036, BMS-986104, BMS-986142, BMS-986171, BMS-986184, BMS-986263, BMT-053011, BMX-010, bortezomib, BOS-580, BOS-161721, BOT-191, BOT-501, branebrutinib, brazikumab (AMG-139), brentuximab vedotin, brepocitinib (PF-06700841), briakinumab, brilaroxazine hydrochloride, brilacidin, brimonidine, brimonidine tartrate, brodalumab, bromocriptine, BT-051, BT-063, BT-104 (LABP-104), BTT-1023, BTX-1204, budesonide, BX-003, BZ-371 Å, C-82, calcipotriol, calcitriol, canakinumab, cannabidiol, cannabidiol+dronabinol, Carbon monoxide (inhaled), carotegrast methyl, Cartistem, CAT-2003, CBP-307, CBS-004, CBS-001, CBW-511, CBX-111, CC-90005, CC-90006, CCL-20LD, CCX-354, CCX-507, CD-10367, CD24-IgFc, CD-4802, CE-1145, celecoxib, CEL-2000, CEL-4000, cenerimod, cenicriviroc, CEQ-508, CER-209, cerdulatinib, certolizumab, certolizumab pegol, CF-101, CF-102, CFZ-533, CGS21680, ChAdOx2-HAV, Chanllergen, choline salicylate, CHR-5154, cibinetide, ciclosporin, cilofexor tromethamine (GS-9674), Cinryze, CIT-013, CJ-16871, CJM-112, CKBA, CKD-506, CKD-971, clazakizumab, clobetasol propionate, clobetasol propionate+tretinoin, Clostridium butyricum, CM-101, CM-2489, CNTO-6785, CNX-014, CNX-023, CNX-024, CNX-025, cobiprostone, cobitolimod, colesevelam, corticotropin, cortisone, cotadutide, COV-08-0064, CPD-1, CPL-409116, CR-6086, CreaVax-RA, crisaborole, CS-12192, CSL-964, CT-327, CT-388, CT-868, CT-859, CT-P13, CTX-101, CU-06, cudetazestat (PAT-505, PAT-048), CUG-252, CWG-92, CWG-940, Cx-611, cyclosporin, CYP-001, Cytori Cell Therapy, CYTX-100, dabigatran etexilate mesylate, dalazatide, danuglipron, dapagliflozin, dapiglutide, dapirolizumab pegol, darvadstrocel, daxdilimab, dazodalibep (VIB4920), DBI-001, DCB-SLE1, DCR-LIV1, DD-01, DD-02, DD-03, DE-098, Debio-0512, deflazacort, delgocitinib, DEN-181, denosumab, desonide, deucravacitinib (BMS-986165), deuterated pioglitazone R-enantiomer, dexamethasone sodium phosphate, DFD-06, diacerein, diclofenac, diclofenac sodium+misoprostol, diflunisal, digeranyl bisphosphonate (OX-14), dilanubicel, dimethyl fumarate, diroleuton, dithranol, DLX-105, DNVX-078, doconexent, dolcanatide, dornase alfa, dorzagliatin, DP-001, DRX-065, DS-102, DS-7011, DSM-9843, DST-0058, DSXS-1411, DSXS-1535, DSXS-1538b, dual inhibitors of sphingosine kinase 1 and 2 (SPHK1/2), dulaglutide, DUR-928 (larsucosterol), DV-1079, DWJ-211, DWJ-1421, DWP-212525, DWP-213388, DZ-2002, DZ-4001, E-6011, E-6742, EB-7020, elafibranor, Elixcyte™,elsubrutinib, EM-101, EMI-400, emricasan, enalapril, endoglin peptides, EN-1001, EN-2001, ENERGI-F704, enpatoran (M-5049), epratuzumab, ertugliflozin, etrasimod, etanercept, etodolac, etoricoxib, etrolizumab, ETX-201, everolimus, evogliptin, exenatide (NLY-01), EYP-001, [18F]F-AraG, F-351, F-652, famitinib L-malate,eculizumab, EDP-1066, EDP-1815, EDP-1867, EDP-305, edratide, efgartigimod alfa, efinopegdutide, efmarodocokin alfa (RG-7880), efpeglenatide, efruxifermin, eicosapentaenoic acid monoglycerides, FB-401, FB-704 Å, FBM-5712, felzartamab, fenebrutinib, fenoprofen calcium, FFP-104, filgotinib, fluasterone, fluocinonide, flurbiprofen, fluticasone propionate, fluvastatin, foralumab, forigerimod, fosdagrocorat, FPP-003, FT-4101, FTP-198, Furestem, GB-004, GDD-3898, GDT-01, GED-0507-34-Levo, gerilimzumab, GH-509, ginsenoside C-K, givinostat, GK-664-S, GKT-831, glutazumab, GL-0034, GL-101, GL-7190, GLG-801, GLPG-0974, GLPG-3121, GLPG-3499, GLPG-3667, GLPG-3970, GLPG-4399,BioChaperone GluExe, GLY-2028, GM-90194, GMDP, GNF-5120, GNKS-356, GNS-1653, goat polyclonal antibodies, gold sodium thiomalate, golimumab, GRI-0124, GRI-0621, GR-MD-02, GS-300, GS-4059, GS-4875, GS-4997, GS-5290, GS-5718, GS-5745, GS-9674, GSK-1070806, GSK-2646264, GSK-2800528, GSK-2831781, GSK-2981278 Å, guselkumab, GX-101, GX-G6, GZR-18, H4 antagonist, halobetasol propionate, halofuginone, halomethasone, HAT-1, HD-7671, HEC-73077, HEC-88473, HEC-96719, Hemay-007, HHT-109, HLD-400, HM-15211, HM-71224, HMPL-004, HMPL-523, HNC-664, HPN-01HR-17031, HRX-0215, HS-10356, HS-20004 (SHR-20004), HS-20094, HSD17b13 inhibitors, HSG-4112, HST-003, HST-201, HST-202, HTD-1801, HTD-2802, HU-6, HuL-001, hyaluronate sodium, hydrochlorothiazide, hydroxychloroquine, (S)-hydroxychloroquine, hydroxychloroquine sulfate, hymecromone, ianalumab (VAY-736), iberdomide, iberogast, IBI-112, IB-RA (injectable rheumatoid arthritis), IB-RA (oral rheumatoid arthritis), ibuprofen, ibrutinib, IcanoMAB, icosabutate, icosapent ethyl ester, ICP-022, ICP-330, ICP-488, ID-11052, IDL-2965, IDN-7314, ifetroban, iguratimod, IL-2 Al Synthorin, IL-23R-CAR-Treg cell therapy, ILT-101, IMD-2560, IMG-003, IMG-007, imidazole salicylate, IN-124-E, IMM-H013, IMO-3100, IMO-8400, imotope, IMS-001, imsidolimab, IMSUT-CORD, IMX-120, INCB-54707, indomethacin, inecalcitol, infliximab, INN-108, inolimomab, INSIX RA, INT-767, interferon gamma, interking recombinant human interleukin-2, interleukin-2, interleukin-2 (injectable), INV-103, INV-17, INV-202, INV-240, IOA-289, ION-224, IONIS-DGAT2Rx, IONIS-JBI1-2.5Rx, IQ-004, ipragliflozin, IR-444, IR-501, IR-502, Irbesarta, IRX-4204, ISB-830, iscalimab, ISM004-1057D, itacitinib, itolizumab, IVA-337, ixekizumab, izencitinib (TD-1473), J2H-1702, JKB-121, JKB-122, JMT-601, JN-2528, JNJ-40346527, JNJ-4447, JNJ-55920839, JNJ-64304500, JNJ-66525433, JNJ-75220795, LEO-152020 (JW1601), julemic acid, K(D)PT, Ka Shu Ning, KAG-308, Kallikrein 7 inhibitors, Kallikrein inhibitors, KB-295, KB-312, KB-GE-001, KBL-697, KBLP-004, KBLP-009, KBP-042, KD-025, KDDF-201110-06, KDDF-201312-11, ketoprofen, ketoprofen+omeprazole, KHK-4083, KHK-4323, KINE-101, KPG-818, KPL-404, KRL507-031, KRP-203, KT-A522, KY-1005, KZR-616, LA-1, lanraplenib, LABP-69, laquinimod, larazotide acetate, LAS-41004, LB-600, LB-700, LBS-009, LBT-6030, LC-200, LC-280126, LC-510255, LD-09163, leflunomide, lenzilumab, LEO-124249, LEO-29102, LEO-32731, LEO-35299, leronlimab (PRO-140), LFS-829, licogliflozin bis(prolinate), linagliptin, liraglutide, lithium succinate, lixisenatide, LLDT-8, LM-002, LM-011, LM-022, LMB-763, LNK-01001, LNP-1892, LNP-1955, LP-02, LP-0200, LTI-03, lumiracoxib, LY-3305677, LY-3090106, LY-3361237, LY-3437943, LY-3457263, LY-3462817, LY-3502970, LY-3493269, LYC-30937 EC, LYN-100, LYS-006, M-1095, M450, M780, M790, MaaT-013, MAdCAM targeted DK4/10, magnesium salicylate, masitinib, mavrilimumab, maxacalcitol, MB-204, MB-N-008, MBS-2320, MBX-8025, mCLC-846, MDV-4463, MDX-018, MEDI-5117, MEDI-8367, meloxicam, mercaptamine, mercaptopurine, mesalazine, MET-2, MET-409, MET-642, metformin, methotrexate, MGD-010, MG-K10, MGL-3196, MGL-3745, MG-S-2525, MHV-370, microRNA-targeting antisense oligonucleotide therapy, midismase, milategrast (E-6007), milatuzumab, mirikizumab (LY-3074828), misoprostol+diclofenac, mitochondrial uncouplers, mizoribine, MK-2060, MM-A01-01, MMI-0100, MOL-4249, momelotinib, mometasone, monalizumab, MORAb-022, MORF-057, MP-1032, MP-117, MP-301, MPC-300-IV, MRC-375, MRx-0006, MS-392, MSB-01, MSB-03, MSCTC-0010, MSDC-0602K, MT-1002, MVA-HAV, MW-04, mycophenolate mofetil, mycophenolate sodium, myristyl nicotinate, nabumetone, nalotimagene carmaleucel, naltrexone, namacizumab, namilumab, naproxen, naproxen sodium, naproxen+esomeprazole, naproxen+esomeprazole strontium, narsoplimab (OMS-721), natalizumab, NAV-003, NC-101, NCT-10004, NDI-010976 (firsocostat), NecroX-5, neihulizumab, Neo-Kidney Augment™ (NKA), NGM-282, NGM-313, NGM-386, NGM-395, niclosamide, nidufexor (LMB-763), NIK-SMI1, nilotinib, nintedanib, NIP-046, NIPEP-APF19, nipocalimab, nitric oxide, NKTR-35, NKTR-358 (LY-3471851), NLP-91, NM-002, NN-6177, norursodeoxycholic acid, NOS-1244, NP-000888, NP-011, NP-135, NP-160, NP-251, NRF-803, NS-200, NTG-A-009, NTR-441, NUE-20798, NV-422, NV-556, NVN-1000, NVP-022, NX-13, NXP-002, NXP-004, 0-304, OATD-01, obeticholic acid, obexelimab, obinutuzumab, ocaratuzumab, ocrelizumab, Octagam 10%, Octanorm 16.5%, ofatumumab, OHR-118, olamkicept, olendalizumab, olesoxime, olitigaltin, olokizumab, olopatadine, olsalazine, OM-89, omiganan pentahydrochloride, omilancor (BT-11), once-daily naproxen (oral controlled release, pain), ONO-4059, Oralgam, ORBCEL-M, ORBCEL-C, orelabrutinib, ORG-129, ORG-447, orilotimod, ORMD-0801, ORMD-0901, OSE-127, Oshadi D, OST-122, otilimab (GSK-3196165), OTL-104, OvaSave, oxaprozin, OXY-210, ozanimod, ozoralizumab, P-007, P-11, P-28-GST, P-3072, P-3073, pacritinib, PAD4 inhibitors, Panzyga®, PAR-2 inhibitors, PAT-1657, pamrevlumab, PB-1023, PB-119, PB-718, PBI-100, PBI-4610, Pc4, PKC theta inhibitors, pefcalcitol, peficitinib, pegapamodutide, peg-ilodecakin, PEG-loxenatide, pegozafermin, PEGylated HLA-x (SLE), pelubiprofen, pexelizumab, PF-4236921, PF-05006739, PF-05285401, PF-06480605, PF-06687234, PF-06835375, PF-547659, PF-07081532, PFI-102, PG-011, PH-46-A, PHN-033, pioglitazone, piperidone hydrochloridum, pirfenidone, piroxicam, pitavastatin, plecanatide, PLN-74809, PLX-1, PN-235, PN-10943, PNPLA3 inhibitor, ponesimod, PR-600, PRA-023, PRAMExe, pravastatin, prednisolone, prednisolone sodium phosphate, prednisone, PRI-724, Procell, Progenra, propagermanium, propionyl-L-carnitine, Prosorba, ProTmune, PRT-2607, PRTX-100, Prurisol, PRV-3279, PRX-003, PRX-167700, PT-101, PTG-100, PTG-200, PUR-0110, PUR-1800, PUR-5700, PVA-N11, Px-102, PX20606, PX-L493, PX-L603, PXL-065, PXS-4728 Å, pyrrolopyrrolyl isoquinoline derivatives, PZ-235, PZH-2109, QBECO, QBSAU, QPI-1002, QU-100, quetmolimab, QX-004-N, QX-006-N, R-2187, rabeximod, RA-Curcusome, razuprotafib, RBX-2660, RBX-8225, RCT-18, RCYM-001, RDX-009, recombinant human interleukin-1 receptor antagonist (rheumatoid arthritis), recombinant human interleukin-2 recombinant TNF receptor 2-Fc fusion protein mutant, recombinant human LFA-3/antibody fusion protein, REGEND-001, remestemcel-L, remogliflozin etabonate, rencofilstat, RES-529, Resunab (JBT-101), RG-125, RG-6125, RG-6287, RG-7835, RGI-2001, RHB-104, Rheumavax, R-HSC-010, RhuDex, ribaxamase, rifabutin+clarithromycin+clofazimine, risankizumab (BI-655066), ritlecitinib (PF-06651600), rituximab, rivogenlecleucel, RO-7486967, R07049665, rodatristat ethyl, rofecoxib, RON-2315, rontalizumab, ROR-gamma T, rose bengal sodium, rosuvastatin, rozibafusp alfa, RP-182, RPI-500, RPI-78, R-salbutamol sulphate, RSLV-132, RT-1840, RTU-1096, ruxolitinib, 5-414114, 5-723595, salvianolic acid, SAN-300, SAN-903, saracatinib, saratin, sarilumab, saroglitazar, SB-012, SBI-087, SBI-3150, SBI-9674, SBP-301, SCM-CGH, SCT-5-27, SDC-1801, SDC-1802, secukinumab, SEFA-1024, seladelpar lysine, seliciclib, selective MCT inhibitor, semaglutide, SER-287, SGF-3, SGM-1019, SGX-301, SH-2442, SHC-023, SHC-028, SHP-141, SHR-0302, sifalimumab, SIG-1456, sildenafil, simtuzumab, simvastatin, sirolimus, sirukumab, SKi-I, SKI-O-703 (cevidoplenib), SM-03, SM-06, SM-934, SM GLP-1, SMET-D1, SMS-0174, SNK-01, SNP-610, SNP-630, sodium nabumetone, sodium pyruvate, sodium salicylate, sodium thiosulfate, solcitinib (GSK-2586184), solithromycin, sotagliflozin, SP-16, SP-14019, SPD-01, spebrutinib, spesolimab (BI-655130), SPH-3127, SR-047, SSS-07, ST-0529, STNM-01, STP-705, Sugaheal variant, sulfasalazine, sulforadex, sulindac, sutimlimab, SYGN-313, Syn-1002, SZ-005, SZN-1326, T-5224, TAB-08, tabalumab, tacalcitol, tacrolimus, tairuimide, TAK-020, TAK-079, TAK-094 (SCO-094), talacotuzumab, T-allol0, TAM-01, tarenflurbil, TAS-5315, tazarotene, TB01-3, TCF-12, TCM-606F, TD-1058, TE-2324, technetium Tc 99m tilmanocept, technetium[99Tc] methylenediphosphonate, teduglutide, telitacicept, telmisartan, tenoxicam, TER-101, TERN-101, TERN-601, TEV-45478, TGF-beta-1/Smad3 signaling inhibitors, theralizumab, Thetanix, thymoglobulin, tildrakizumab, tilorone, tipelukast, tirabrutinib hydrochloride, tirbanibulin, tirzepatide, TJC-031, TJC-0434, TLY-012, TNX-1500, tocilizumab, tofacitinib, tolimidone, tolmetin sodium, tonabacase, TOP-1288, TOP-1890, toreforant, Toritz, TPX-6001, TPX-7001, TQA-3526, TQA-3563, TQG-2813, TR-8, tralokinumab, trans-sodium crocetinate, tregalizumab, Trichuris suis ova, TRK-170, tropifexor, TRX-318, TS-20004, TTP-273, TU-2100, TVB-2640, TXR-612, TXR-711, TXR-712, TZ-101, UBP-1213, UCB-5857, UCB-9741, UD-009, UHE-105, ulobetasol, umbilical cord blood-derived stem cell therapy, umbilical cord-derived mesenchymal stem cells (iv, RA/liver disease), UMC119-06, UN-03, upadacitinib, ursodeoxycholic acid, ustekinumab, valdecoxib, valziflocept, VAR-400, VB-201, VBY-129, VBY-376, VBY-825, VBY-891, vedolizumab, VEGFR targeted DK4/10, Veltuzumab, vemircopan, verdinexor, veverimer, VIB-1116, vidofludimus, vismodegib, VISTA agonist, vitamin D analogs, VK-2735, VK-2809, VNLG-152, vobarilizumab, voclosporin, volixibat potassium ethanolate hydrate, VP-02, VRN-02, VS-105, VTP-43742, VTX-958, VTX-2735, VVP-100X, WAV-301, WBI-1001, WF-10, WNT-974, WXSH-0038, WXSH-0078, X-6, XEN-103, XmAb-5871, XNW-1011, XRx-117, XRx-221, XTYW-003, XW-001, XW-003, XW-004, XZ.700, YH-1713, YH-25724, YH-35324, YHB-1411-2, YPF-1827, YPS-345, YRA-1909, ZeP-3, zevaquenabant, ZG-5216, ZGN-839, zimlovisertib, ZM-008, ZPL-389, ZSP-1603, ZSYM-008, zunsemetinib (ATI-450), ZYBK-2, ZYSM-007, and/or bi-specific antibodies targeting one or more targets referenced herein.

In some embodiments, the antibody or antigen-binding fragment thereof of the disclosure is co-administered with one or more (e.g., one, two, three, or four) additional therapeutic agents. In some embodiments, the additional therapeutic agent includes an agent useful for modulating, treating, or preventing inflammation, such as 5-HT 1a receptor partial agonists and antagonists, 5-HT 2a receptor partial agonists and antagonists, 5-HT 2b receptor antagonists, 5-HT 6 receptor antagonists, 5-HT 7 receptor antagonists, Abl tyrosine kinase inhibitors, ACE inhibitors, Acidic mammalian chitinase inhibitors, Actin antagonists, Acetaldehyde dehydrogenase inhibitors, Acetyl CoA carboxylase (ACC) inhibitors, ACC-1 inhibitors, ACC-2 inhibitors, 2-Acylglycerol O-acyltransferase 2 (DGAT2) inhibitors, ACTH receptor agonists, activin receptor antagonists, adenosylhomocysteinase inhibitors, Adenosine receptor antagonists and agonists, adenosine deaminase inhibitors, Adenylyl cyclase associated protein 1 inhibitors, adiponutrin inhibitors, adiponectin receptor agonists, ADP ribosyl cyclase-1 inhibitors, ADP ribosyl cyclase-1 modulators, ADP ribosylation factor 6 inhibitors, adrenocorticotrophic hormone ligands, adrenomedullin ligands, Adrenergic receptor antagonists and agonists, adropin stimulators, aggrecanase-2 inhibitors, AIMP multisynthetase complex protein 1 stimulators, AKT1 gene inhibitors, AKT protein kinase inhibitors, Albumin antagonists, Albumin modulators, aldehyde dehydrogenase 2 stimulators, Aldosterone antagonists, Aldosterone synthase inhibitors Alk-5 protein kinase inhibitors, alpha 2 adrenoceptor agonists, Alpha 2 adrenoceptor modulators, Alpha 1 antitrypsin stimulator, alpha-fetoprotein modulators, alstrom syndrome protein 1(ALMS1)/PKC alpha protein interaction inhibitors, 1 aminocyclopropane carboxyl synthase inhibitors, amylin receptor agonists, AMP-activated protein kinases (AMPK), AMP activated protein kinase inhibitors, activators or stimulators, AMP activated protein kinase alpha 2 stimulators, androgen receptor agonists and antagonists, angiopoietin-related protein-3 inhibitors, Angiotensin II receptor antagonists, angiotensin II AT-I receptor antagonists, angiotensin II AT-2 receptor agonists, angiotensinogen ligand inhibitors, Annexin Al modulators, antibiotics, antifungals, anti-IL6 antibodies, anti-TNF steroid conjugates, activator protein 1 (AP1) transcription factor inhibitors, AP1 transcription factor modulators, apelin receptor agonists, APOA1 gene stimulators, apolipoprotein A antagonists, apolipoprotein B modulators, apolipoprotein L1 modulators, apoptosis regulator Bcl-w inhibitors, aryl hydrocarbon receptor (AHR) agonists and modulators, AHR agonist plus autoantigen, ASK1 inhibitors, ATPase inhibitors, ATP binding cassette transporter C2 inhibitors, ATP citrate lyase inhibitors, autophagy protein modulators and stimulators, autotaxin inhibitors, Axl tyrosine kinase receptor inhibitors, BAFF/APRIL inhibitors, basigin inhibitors, B and T lymphocyte attenuator stimulators, bax protein stimulators, Bcl-2 protein inhibitors, Bcl-xL Bcl-2 associated death promotor inhibitors, Bcl-xL Bcl-2 associated death promotor modulators, Bcr protein inhibitors, Benzodiazepine receptor agonists, beta adrenoceptor antagonists, BET inhibitors, beta 2 adrenoceptor agonists, beta amyloid antagonists, beta-catenin inhibitors, beta-catenin modulators, beta-catenin stimulators, beta-galactosidase inhibitors, beta lactamase modulators, 17 beta hydroxysteroid dehydrogenase 13 inhibitors, bifunctional aminoacyl tRNA synthetase inhibitors, B-lymphocyte antigen CD19 inhibitors, B-lymphocyte antigen CD20 inhibitors, B-lymphocyte antigen CD20 modulators, B-lymphocyte cell adhesion molecule inhibitors, B-lymphocyte stimulator ligand inhibitors, B-lymphocyte stimulator ligand modulators, bioactive lipids, bone morphogenetic protein-7 ligand, bone morphogenetic protein-7 ligand modulators, bradykinin receptor modulators, braf gene inhibitors, branched amino acid aminotransferase 1 inhibitors, bromodomain containing protein (BRD) inhibitors, BRD1, BRD2, and BRD4 inhibitors, BTK inhibitors, B7 homolog inhibitor, cadherin-11 antagonists, Cak tyrosine kinase receptor inhibitors, calcineurin inhibitors, calcium channel inhibitors, Ca2+release activated Ca2+channel 1 inhibitors, calcitonin agonists, calpain-IX inhibitors, calpain-I inhibitors, calpain-II inhibitors, calreticulin inhibitors, caveolin 1 stimulators, cannabinoid CB1 receptor antagonists and inverse agonists, cannabinoid CB2 receptor agonists, cannabinoid receptor antagonists and agonists, cannabinoid CB1 receptor inverse agonists, carbohydrate metabolism modulators, carbonic anhydrase inhibitors, casein kinase-I delta and/or epsiloninhibitors, CASP9 gene stimulators, caspase inhibitors, caspase-3 stimulators, catalase stimulators, cathepsin inhibitors, cathepsin K inhibitors, cathepsin S inhibitors, caveolin 1 inhibitors, CCK receptor antagonists, CCAAT enhancer binding protein beta modulators, C-C motif ligand 26 (CCL26) gene inhibitors, chemokine receptor antagonists, C-C motif chemokine receptor (CCR) 1 antagonists, CCR2 antagonists, CCR3 antagonists and modulators, CCR4 antagonists, CCR5 antagonists, CCR6 antagonists, CCR7 modulators, CCR9 chemokine antagonists, CCR3 gene modulators, CD3 modulators or antagonists, CD4 agonists or antagonists, CD7 inhibitors, CD11b agonists, CD29 modulators, CD39 agonists, CD40 ligand receptor modulators or antagonists, CD47 antagonists, CD52 antagonists, CD73 agonists and antagonists, CD79b modulators, CD80 modulators or antagonists, CD86 modulators or antagonists, CD95 antagonists, CD126 antagonists, CD223 modulators, CDGSH iron sulfur domain protein modulators, CDw123 antagonists, Cell adhesion molecule inhibitors, cell surface glycoprotein CD200R agonists, cell surface glycoprotein MUC18 inhibitors, chemokine CXC ligand inhibitors, chaperonin inhibitors and modulators, chitinase inhibitors, chitotriosidase 1 inhibitors, chloride channel stimulators, cholera enterotoxin subunit B inhibitors, choline kinase inhibitors, CHST15 gene inhibitors, chymase inhibitors, claudin 1 inhibitors, clusterin stimulators, CNR1 inhibitors, collagen I antagonists, collagen VII antagonists, collagen gene inhibitors, collagenase inhibitors, collagen modulators, complement Clq subcomponent inhibitors, complement CIs subcomponent inhibitors, complement C3 inhibitors, complement C5 factor inhibitors, complement C5a receptor antagonists, complement cascade inhibitors, complement factor stimulators, complement factor B inhibitors, complement factor D inhibitors, connective tissue growth factor ligand inhibitors, corticosteroid hormone receptor agonists, COT protein kinase inhibitors, CREB binding protein inhibitors, C-reactive protein (CRP) inhibitors, cerebrospinal fluid (CSF)-1 agonists and antagonists, C-type lectin domain protein 4C inhibitors, CTGF gene inhibitors, CX3CR1 antagonists and modulators, CXCR2 antagonists, CXCR3 antagonist, CXCR4 antagonists and modulators, CXCR5 antagonists and modulators, CXC5 ligand inhibitors, CXC6 chemokine ligand inhibitors, CXC10 ligand inhibitors, CXC 11 ligand modulators, Cyclin-dependent kinase (CDK) 1, 2, 5, 7, and/or 9 inhibitors, Cyclooxygenase (COX) inhibitors, COX-1 inhibitors, COX-2 inhibitors and modulators, cysteine palmitoyltransferase porcupine inhibitors, cytochrome P450 7A1 inhibitors, cytochrome P450 11B2 inhibitors, cytochrome P450 2E1 inhibitors (CYP2E1), cytochrome P450 reductase inhibitors, cytokine receptor agonists and antagonists, Cytosolic phospholipase A2 (cPLA2) inhibitors, Cytotoxic T-lymphocyte protein-4 (CTLA4) modulators and stimulators, Deoxyribonuclease (DNase) modulators, DNase gamma stimulators, DNase I stimulators, DGAT2 gene inhibitors, DHFR inhibitors, Diacylglycerol O acyltransferase (DGAT) 1 inhibitors, DGAT2 inhibitors, Diamine acetyltransferase inhibitors, Dihydroceramide delta 4 desaturase inhibitors, Dihydroorotate dehydrogenase inhibitors, Dipeptidyl peptidase (DPP)I inhibitors, DPP IV inhibitors, DNA binding protein Ikaros inhibitors, DNA methyltransferase inhibitors, DNA polymerase inhibitors, Dopamine D2 receptor partial agonists, Dopamine D3 receptor partial agonists, Dopamine D4 receptor partial agonists, Dopamine D2 receptor agonists, DYRK-1 alpha protein kinase inhibitors, Ectonucleotide pyrophosphatase-PDE-2 inhibitors, EGFR tyrosine kinase receptor inhibitors, EGR1 gene inhibitors, elongation factor 2 inhibitors, endoglin inhibitors, endoplasmin inhibitors, endosialin modulators, endostatin modulators, endothelin ET-A receptor antagonists, endothelin ET-B receptor antagonists, Endothelial nitric oxide synthase stimulators, Enolase 1 inhibitors, Enteropeptidase inhibitors, Eotaxin 2 ligand inhibitors, eotaxin ligand inhibitors, EP4 prostanoid receptor antagonists or agonists, EP4 prostanoid receptor antagonists, Epidermal growth factor (EGF) receptor antagonists, EGF modulators, Epoxide hydrolase inhibitors, Erythropoietin receptor antagonists or agonists, Exportin 1 inhibitors, Extracellular matrix protein modulators, FiFO ATP synthase modulators, Facilitated glucose transporter-1 modulators, Factor IIa antagonists, Factor XIIa antagonists, Farnesoid X receptor (FXR) agonists and modulators, Fatty acid synthase inhibitors, fecal microbiota transplantations (FMT), fibroblast activation protein (FAP) inhibitors, Fibroblast growth factor (FGF) receptor agonists and antagonists, FGF-2 ligand inhibitors, FGF1 receptor agonists and antagonists, FGF2 receptor antagonists, FGF3 receptor antagonists, FGF19 gene stimulators, FGF-15 ligands or modulators, FGF-19 ligands or modulators, FGF-21 ligands or modulators, FK506 binding protein inhibitors, FK506 binding protein-10 inhibitors, FK506 binding protein-12 modulators, Flt3 tyrosine kinase inhibitors, Focal adhesion kinase inhibitors, Folate antagonists or agonists, Folate receptor beta antagonists, FP prostanoid receptor antagonists, Fractalkine ligand inhibitors, Free fatty acid receptor 1, 2, and/or 3 agonists, free fatty acid receptor 2 antagonists, Frizzled-5 receptor agonists, Frizzled-8 receptor agonists, Fyn tyrosine kinase inhibitors, G-protein coupled bile acid receptor 1 agonists, G protein coupled receptor 15 antagonists, G-protein beta subunit inhibitors, G-protein coupled receptor (GPCR) 35, 44, 84, 119, 120 modulators, GPCR 44, 87 antagonists, GABA A receptor modulators, GABA A receptor alpha-2 subunit modulators, GABA A receptor alpha-3 subunit modulators, Galanin GAL2 receptor agonists, Galectin-3 inhibitors, Gastric inhibitory polypeptide receptor (GIP-R) agonists and modulators, GATA 3 transcription factor inhibitors, GDNF family receptor alpha like agonists, GHR gene inhibitors, Glucagon-like peptide (GLP) 1 agonists, GLP 2 agonists, GLP 1 receptor modulators, Glucocorticoid agonists or antagonists, Glucocorticoid induced leucine zipper stimulators, Glucokinase stimulators, Glucose 6-phosphate 1-dehydrogenase inhibitors, Glutaminyl peptide cyclotransferase inhibitors, Glutaredoxin 1 modulators, Glutathione dependent PGD synthase inhibitors, Glycoprotein Ib (GPIb) antagonists, GM-CSF receptor antagonists or modulators, GMP synthetase inhibitors, GNRH receptor modulators, GP IIb IIIa antagonists, GPCR modulators, GPR40 agonists, GPR84 antagonists, GroEL protein 2 inhibitors, GroEL protein 2 inhibitors, Growth hormone ligands, Growth hormone receptor agonists, Growth regulated protein alpha ligand inhibitors, guanylate cyclase receptor agonists, Guanylate cyclase stimulators, Heat shock protein inhibitors, H+K+ATPase inhibitors, Hedgehog (Hh) modulators, Hh protein inhibitors, Heme oxygenase 1 modulators, Hepatitis B structural protein inhibitors, Hepatitis C virus NS3 protease inhibitors, Hepatitis C virus protein NS5A inhibitors, Hepatocyte nuclear factor 4 alpha modulators (HNF4A), Hepatocyte growth factor modulators and antagonists, hypoxia inducible factor (HIF) prolyl hydroxylase inhibitors, HIF prolyl hydroxylase-2 inhibitors, High mobility group protein B1 inhibitors, Histamine H1 receptor antagonists, Histamine H4 receptor agonists, Histamine H4 receptor antagonists, Histamine H4 receptor modulators, Histone deacetylase (HDAC) inhibitors, HDAC-1 inhibitors, HDAC-2 inhibitors, HDAC-3 inhibitors, HDAC-6 inhibitors, H+K+ATPase inhibitors, HIV-1 gpi20 protein inhibitors, HLA antigen modulators, HLA class II antigen DQ-2 alpha modulators, HLA class II antigen DR-1 beta inhibitors, HLA class II antigen inhibitors, HLA class II antigen modulators, HMG CoA reductase inhibitors, Homeodomain interacting kinase 2 (HIPK2) inhibitors, Hormone sensitive lipase stimulators, HSD17B3 gene modulators, HSD17B13 gene inhibitors, Hsp 70 family inhibitors and stimulators, Hsp 90 inhibitors, Hyaluronidase stimulators, Hydrolase inhibitors, Hypoxia inducible factor (HIF) modulators, HIF-1 inhibitors, HIF-1 alpha modulators and stimulators, HIF-2 alpha inhibitors, ICAM1 gene inhibitors, ICE inhibitors, interferon beta (IFNB) gene stimulators, Insulin-like growth factor 1 (IGF1) gene inhibitors, IgG receptor FcRn large subunit p51 antagonists, IgG receptor FcRn large subunit p51 modulators, I-kappa B kinase inhibitors, I-kappa B kinase beta inhibitors, IK potassium channel inhibitors, Interleukin (IL)-1 antagonists, IL-2 agonists or antagonists, IL-3 antagonists, IL-4 agonists or antagonists, IL-5 antagonists, IL-6 agonists or antagonists, IL-7 receptor antagonists, IL-8 antagonists, IL-10 antagonists or agonists, IL-11 agonists, IL-12 antagonists, IL-13 antagonists, IL-15 antagonists, IL-17, IL17 Å, and IL17B agonists or antagonists, IL-18 antagonists, IL-21 antagonists, IL-22 agonists or antagonists, IL-23 antagonists, IL-1 beta ligand modulators, IL-23A inhibitors, IL-31 receptor modulators and antagonists, IL-36 inhibitors, IL-6 neutralizing human antibodies, IL-1 receptor accessory protein inhibitors, IL-18 receptor accessory protein antagonists, IL-2 receptor alpha subunit inhibitors, IL-2 receptor alpha subunit stimulators, Interleukin ligands, IL-1 alpha ligand inhibitors, IL-1 ligand inhibitors, IL-1 beta ligand inhibitors and modulators, IL-1 beta ligands, interleukin ligand inhibitors, IL-2 ligands, IL-4 ligands, IL-4 ligand inhibitors, IL-6 ligand inhibitors, IL-8 ligand inhibitors, IL-10 ligands, IL-13 ligand inhibitors, IL 17 ligand inhibitors, IL 17A ligand inhibitors and modulators, IL-17F ligand inhibitors, IL 18 ligand inhibitors, Interleukin-22 ligands, IL-29 ligands, IL-33 ligand inhibitors, IL-1 like receptor inhibitors, Ileal sodium bile acid cotransporter inhibitors, immunoglobulin (Ig) agonists or antagonists, IgE antagonists and modulators, Immunoglobulin Fc receptor modulators, IgG agonists, IgGI agonists and antagonists, IgG2 antagonists and modulators, Immunoglobulin gamma Fc receptor antagonists, Immunoglobulin gamma Fc receptor II modulators, Immunoglobulin gamma Fc receptor IIB antagonists, Immunoglobulin kappa modulators, Immunoglobulin like domain receptor 2 antagonists, IgM antagonists, Inducible nitric oxide synthase inhibitors (iNOS inhibitors), Inducible T-cell co-stimulator inhibitors, Inosine monophosphate dehydrogenase inhibitors, Insulin ligands, Insulin ligand agonists, Insulin receptor agonists, Insulin receptor substrate-1 inhibitors, Insulin sensitizers, integrin antagonists and modulators, Integrin alpha-1/beta-1 antagonists, Integrin alpha-4/beta-1 antagonists, Integrin alpha-V/beta-1 antagonists, Integrin alpha-V/beta-3 antagonists, Integrin alpha-V/beta-6 antagonists, Integrin alpha-V/beta-8 modulators, integrin alpha-4/beta-7 antagonists, Integrin alpha-9 antagonists, Interferon (IFN) alpha ligands, IFN alpha ligand inhibitors and modulators, IFN omega ligand inhibitors, IFN beta ligands, IFN beta ligand inhibitors, IFN gamma ligands, IFN gamma receptor 1 agonists, IFN gamma receptor antagonists, IFN type I receptor antagonists, interleukin-1 receptor-associated kinase 4 (IRAK4) inhibitors, IRE1 protein kinase inhibitors, Itk tyrosine kinase inhibitors, Janus Kinase (JAK) inhibitors and modulators, JAK3 gene inhibitors, JAK1 inhibitors, JAK2 inhibitors, JAK3 inhibitors, Jun N terminal kinase inhibitors, Jun N terminal kinase-1 inhibitors, Kallikrein inhibitors, Kallikrein 2 inhibitors, Kallikrein 7 inhibitors, KCNA voltage-gated potassium channel-3 inhibitors, KCNA voltage-gated potassium channel-3 modulators, KCNN potassium channel-4 inhibitors, KCNN4 gene inhibitors, Kelch like ECH associated protein 1 modulators, Ketohexokinase (KHK) inhibitors, Kit tyrosine kinase inhibitors, Klotho beta stimulators, lactoferrin stimulators, LanC like protein 2 stimulators, LanC like protein 2 modulators, Lek tyrosine kinase inhibitors, LDHA gene inhibitors, LDL receptor related protein-1 stimulators, LDL receptor related protein-6 inhibitors, LDL receptor related protein-6 stimulators, Lectin mannose binding protein inhibitors, leukocyte elastase inhibitors, Leukocyte Ig like receptor A4 modulators, leukocyte proteinase-3 inhibitors, Leukotriene receptor antagonists, Leukotriene A4 hydrolase inhibitors, Leukotriene BLT receptor antagonists, Leukotriene D4 antagonists, 5-Lipoxygenase activating protein inhibitors, 5-Lipoxygenase inhibitors, Lipoxygenase modulators, Lipoprotein lipase inhibitors, LITAF gene inhibitors, Liver X receptor agonists and antagonists, Liver X receptor alpha inverse agonists, Liver X receptor beta inverse agonists, LPL gene stimulators, Lymphocyte function antigen-3 receptor antagonists, Lyn tyrosine kinase inhibitors, Lyn tyrosine kinase stimulators, Lysophosphatidate-1 receptor antagonists, Lysyl oxidase homolog (LOXL) 2 inhibitors, LXR inverse agonists, macrophage-drug conjugates (MDC), Macrophage inflammatory protein (MIP) 2 alpha inhibitors, MIP 2 beta inhibitors, MIP 3 alpha ligand inhibitors, Macrophage mannose receptor 1 modulators, Macrophage migration inhibitory factor inhibitors, MAdCAM inhibitors, MAdCAM modulators, MALT protein 1 inhibitors, Mannan-binding lectin serine protease-2 inhibitors, MAP kinase inhibitors, MAP kinase kinase 4 inhibitors, MAP kinase modulators, MAP3K2 gene inhibitors, MAPKAPK2 inhibitors, MAPKAPK5 inhibitors, Matrix extracell phosphoglycoprotein modulators, Matrix metalloprotease inhibitors, MCH receptor-1 antagonists, MCL1 gene inhibitors, MEK protein kinase inhibitors, MEK-1 protein kinase inhibitors, MEK-2 protein kinase inhibitors, MEKK-5 protein kinase inhibitors, melanin concentrating hormone (MCH-1) antagonists, melanocortin agonists, Melanocortin MC1 receptor agonists, Melanocortin MC3 receptor agonists, Melanocortin receptor agonists, Membrane copper amine oxidase inhibitors, Metalloprotease-1 inhibitors, Metalloprotease-2 inhibitors, Metalloprotease-9 inhibitors, Metalloprotease-9 stimulators, methylprednisolone, Methionine aminopeptidase-2 inhibitors, Methyl CpG binding protein 2 modulators, microbiome-targeting therapeutics, MicroRNA-132 (miR-132) antagonists, MicroRNA-21(miR-21) inhibitors, Midkine ligand inhibitors, Mineralocorticoid receptor antagonists and modulators, Mitochondrial uncouplers, Mitochondrial 10 kDa heat shock protein stimulators, Mitochondrial pyruvate carrier 2 inhibitors, Mitochondrial pyruvate carrier inhibitors, Mixed lineage kinase-3 inhibitors, MKL myocardin like protein inhibitors, MNK protein kinase inhibitors, Monocarboxylate transporter inhibitors, Monocyte macrophage differentiation inhibitors, Motile sperm domain protein 2 inhibitors, MST-1 protein kinase inhibitors, mTOR complex 1 inhibitors, mTOR complex 2 inhibitors, mTOR inhibitors, Myelin basic protein stimulators, Myeloperoxidase inhibitors, Myosin 2 inhibitors, N-formyl peptide receptor antagonists, NACHT LRR PYD domain protein 3 (NLRP3) inhibitors, NAD ADP ribosyltransferase stimulators, NAD-dependent deacetylase sirtuin stimulators, NAD-dependent deacetylase sirtuin-1 stimulators, NADPH oxidase inhibitors, NADPH oxidase 1 inhibitors, NADPH oxidase 4 inhibitors, NAMPT gene inhibitors, natriuretic peptide receptor C agonists, neuregulin-4 ligands, Neuropilin 2 modulators, Neutral endopeptidase inhibitors, NF kappa B inhibitor stimulators, NFAT gene inhibitors, NFE2L2 gene inhibitors, NFE2L2 gene stimulators, Nicotinic acetylcholine receptor antagonists, Nicotinic acid receptor 1 agonists, Nicotinamide phosphoribosyltransferase inhibitors, NK cell receptor modulators, NK1 receptor antagonists, NKG2 A B activating NK receptor antagonists, NKG2 D activating NK receptor antagonists, NLR family member X1 stimulators, NLRP3 inhibitors, NMDA receptor epsilon 2 subunit inhibitors, NOD2 gene modulators, Non receptor tyrosine kinase TYK2 antagonists, NOX4 gene inhibitors, NUAK SNF1-like protein kinase 1 inhibitors, Nuclear erythroid 2-related factor 2 stimulators, Nuclear factor kappa (NFK) B inhibitors and modulators, Nuclear factor kappa B p105 inhibitors, nuclear hormone receptor modulators, Nuclear pore complex protein modulators, Nuclear receptor modulators, Nuclease stimulators, Nucleoside reverse transcriptase inhibitors, Nucleosome assembly protein 1 like-4 inhibitors, Oncostatin M receptor modulators, Oncostatin M receptor subunit beta inhibitors, opioid receptor antagonists, Opioid growth factor receptor agonists, Opioid receptor delta, kappa, and mu antagonists, Opioid receptor sigma antagonist 1, Orphan nuclear receptor antagonists, Osteoclast differentiation factor antagonists, Osteoclast differentiation factor ligand inhibitors, Oxidoreductase inhibitors, OX40 ligand inhibitors, OX-40 receptor antagonists and modulators, Oxyntomodulin ligands, PGE1 agonists, P-Glycoprotein inhibitors, P-selectin glycoprotein ligand-1, 14-3-3 protein eta inhibitors, P2X3 purinoceptor antagonists, P2X7 purinoceptor agonists and modulators, P2Y6 purinoceptor modulators, P2Y13 purinoceptor stimulators, p38 MAP kinase alpha inhibitors, p38 MAP kinase inhibitors, p53 tumor suppressor protein stimulators, PACAP type I receptor agonists, Pan cathepsin inhibitors, Parathyroid hormone ligand inhibitors, PARP modulators, PDE 1 inhibitors, PDE 3 inhibitors, PDE 4 inhibitors, PDE 4b inhibitors, PDE 5 inhibitors, PDGF-B ligand inhibitors, PDGF receptor agonists, PDGF receptor alpha antagonists, PDGF receptor beta antagonists and modulators, PEGylated long-acting glucagon-like peptide-1/glucagon (GLP-1R/GCGR) receptor dual agonists, Pellino homolog 1 inhibitors, Peptidyl-prolyl cis-trans isomerase A inhibitors, Peptidyl-prolyl cis-trans isomerase D inhibitors, PERK gene inhibitors, PGI2 agonists, PGD2 antagonists, Phenylalanine hydroxylase stimulators, Phosphatidylinositol 3 kinase subunit 3 inhibitors, Phosphatonin receptor agonists, Phosphoinositide 3-kinase inhibitors, Phosphoinositide-3 kinase alpha, delta, and gamma inhibitors, Phospholipase A2 inhibitors, Phospholipase C inhibitors, Phosphoric diester hydrolase inhibitors, Phosphorylase inhibitors, Plasma retinol binding protein inhibitors, Plasminogen activator inhibitor 1 inhibitors, Plasmin stimulators, Platelet activating factor receptor antagonists, Plexin domain containing protein stimulators, PNPLA3 gene inhibitors and modulators, Potassium channel inhibitors PPAR agonists, PPAR alpha/delta agonists, PPAR delta agonists, PPAR gamma agonists and modulators, PRKAA2 gene stimulators, Programmed cell death ligand (PDL) 1 modulators, Programmed cell death protein 1 modulators, Programmed cell death protein 1 stimulators, Proprotein convertase PC9 inhibitors, Prostacyclin (PGI2) agonists, Prostaglandin D synthase stimulators, Prostanoid receptor antagonists, Protease-activated receptor-2 antagonists, Proteasome beta-8 subunit modulators, Proteasome inhibitors, Protein arginine deiminase inhibitors, Protein arginine deiminase IV inhibitors, Protein C activators, Protein cereblon modulators, protein fimH inhibitors, Protein kinase C theta inhibitors, Protein kinase inhibitors and modulators, Protein kinase C theta inhibitors, Protein MB21D1 inhibitors and modulators, Protein NOV homolog modulators, P-selectin glycoprotein ligand-1 inhibitors, Protein tyrosine kinase inhibitors, Protein tyrosine phosphatase beta inhibitors, Protein tyrosine phosphatase-1B inhibitors, Protein tyrosine phosphatase-2C inhibitors, Protein tyrosine phosphatase lE inhibitors, P-selectin glycoprotein ligand-1 stimulators, PTGS2 gene inhibitors, PurH purine biosynthesis protein inhibitors, QSK serine threonine protein kinase inhibitors, Ras gene inhibitors, Reactive oxygen species modulator inhibitors, Relaxin receptor modulators, Relaxin receptor 2 modulators, Renin inhibitors, Resistin ligand inhibitors, Resistin/CAP1 (adenylyl cyclase associated protein 1) interaction inhibitors, Retinoic acid receptor agonists, Retinoic acid receptor gamma antagonists and inverse agonists, Retinoid receptor agonists, Retinoid X receptor agonists and modulators, Retinoid Z receptor gamma agonists and antagonists, Ret tyrosine kinase receptor inhibitors, Rev protein modulators, Rho associated protein kinase inhibitors, Rho associated protein kinase 1 inhibitors, Rho associated protein kinase 2 inhibitors, Rhomboid family member 2 inhibitors, Ribonuclease P inhibitors, RIP-1 kinase inhibitors, RIP-2 kinase inhibitors, RNA polymerase inhibitors, Seprase inhibitors, Serine threonine protein kinase TBK1 inhibitors, Serine threonine protein kinase TBK1 modulators, Serine threonine SNF1 like kinase 2 inhibitors, SERPINHI gene inhibitors, Serum amyloid A protein modulators, Serum amyloid P stimulators, Signal transducer CD24 modulators, Signal transduction inhibitors, SLC22A12 inhibitors, SMAD inhibitors, SMAD-3 inhibitors, Smoothened receptor antagonists, S-nitrosoglutathione reductase (GSNOR) enzyme inhibitors, Sodium channel inhibitors, Sodium glucose transporter-1 inhibitors, Sodium glucose transporter-2 inhibitors, Solute carrier family inhibitors, Somatostatin receptor agonists, Sphingolipid delta 4 desaturase DES1 inhibitors, Sphingosine kinase 1 inhibitors, Sphingosine kinase 2 inhibitors, Sphingosine 1 phosphate phosphatase modulators, sphingosine 1 phosphate phosphatase 1 stimulators, sphingosine-1-phosphate receptor-1 agonists, sphingosine-1-phosphate receptor-5 agonists, sphingosine-1-phosphate receptor-1 antagonists, sphingosine-1-phosphate receptor-1 modulators, Sphingosine-1-phosphate receptor-3 modulators, Sphingosine-1-phosphate receptor-4 modulators, Sphingosine-1-phosphate receptor-5 modulators, Src tyrosine kinase inhibitors, SREBP transcription factor inhibitors, SREBP transcription factor 1 inhibitors, SREBP transcription factor 2 inhibitors, STAT inhibitors, STAT3 gene inhibitors, STAT-1 inhibitors and modulators, STAT-3 inhibitors and modulators, STAT-5 inhibitors, STAT-6 inhibitors, Stearoyl CoA desaturase-1 inhibitors, stem cell antigen-1 inhibitors, Stimulator of interferon genes protein inhibitors, STK25 inhibitors, Stress induced secreted protein 1 stimulators, superoxide dismutase modulators, Superoxide dismutase stimulators, Suppressor of cytokine signalling-1 stimulators, Suppressor of cytokine signalling-3 stimulators, SYK inhibitors, Syndecan-1 inhibitors, TACE inhibitors, TAK1 binding protein modulators, Talin modulators, Taste receptor type 2 agonists, T-box transcription factor TBX21 modulators, T-cell differentiation antigen CD6 inhibitors, T cell receptor modulators, T cell receptor antagonists, T-cell surface glycoprotein CDla inhibitors, T-cell surface glycoprotein CD8 inhibitors, T cell surface glycoprotein CD28 inhibitors, T-cell surface glycoprotein CD8 modulators, T cell surface glycoprotein CD28 stimulators, T-cell transcription factor NFAT modulators, Tec tyrosine kinase inhibitors, Telomerase stimulators, Tenascin modulators, TERT gene modulators, TGF-beta activated kinase-1 inhibitors, TGF-beta activation modulators, TGF beta agonists, TGF beta ligand inhibitors, TGF beta 1 ligand inhibitors, TGF beta 3 ligand inhibitors, TGF beta 1 gene inhibitors, TGF beta 1 ligand modulators, TGF beta receptor antagonists, TGF beta receptor antagonists, TGF-beta type II receptor antagonists, TGFB1 gene inhibitors, Thioredoxin reductase inhibitors, Thrombomodulin stimulators, Thromboxane A2 antagonists, Thromboxane A2 receptor antagonists, Thromboxane synthesis inhibitors, Thymic stromal lymphopoietin ligand inhibitors, Thymic stromal lymphopoietin ligand modulators, Thymic stromal lymphopoietin receptor modulators, Thymulin agonists, Thyroid hormone receptor agonists, Thyroid hormone receptor beta agonists, tissue transglutaminase inhibitors, Toll-like receptor (TLR)-2 antagonists, TLR-3 antagonists, TLR-4 antagonists, TLR-7 antagonists and modulators, TLR-8 antagonists, TLR-9 antagonists and agonists, TLR modulators, TNF alpha ligand agonists and antagonists, TNF ligand agonists and antagonists, TNF binding agents, TNF gene inhibitors, TNFSF11 gene inhibitors, Topoisomerase II inhibitors, TPL-2 inhibitors, Transaminase stimulators, Transcription factor modulators, Transcription factor β65 inhibitors, Transcription factor RelB inhibitors, Transferrin modulators, Transforming growth factor R (TGF-0), Transforming growth factor R activated Kinase 1 (TAK1), Transglutaminase inhibitors, Transthyretin modulators, TrkA receptor antagonists, Trk tyrosine kinase receptor inhibitors, TRP cation channel Al inhibitors, TRP cation channel C5 inhibitors, TRP cation channel C6 inhibitors, Tryptophan 5-hydroxylase-1 inhibitors, Tryptophanase inhibitors, Tubulin binding agents, Tumor necrosis factor ligand inhibitors, Tumor necrosis factor ligand 13 inhibitors, Tumor necrosis factor 15 ligand inhibitors, tumor necrosis factor 14 ligand modulators, Tumor necrosis factor 13C receptor antagonists, Tumor necrosis factor 14 ligand inhibitors, Tyk2 tyrosine kinase inhibitors, Type I IL-1 receptor antagonists, Type I TNF receptor antagonists, Type II TNF receptor antagonists, Type II TNF receptor modulators, Tyrosine kinase receptor inhibitors, Tyrosine kinase receptor modulators, Ubiquitin ligase modulators and stimulators, Ubiquitin thioesterase-30 inhibitors, Uncoupling protein modulators, Unspecified cell adhesion molecule inhibitors, Unspecified GPCR agonists, Unspecified GPCR modulators, Unspecified growth factor receptor antagonists, Urate anion exchanger 1 inhibitors, vanilloid VR1 agonists, Vanilloid VR1 antagonists, Vasopressin Vla receptor antagonists, VDR agonists, VEGF receptor antagonists, VEGF receptor modulators, VEGF-1 receptor antagonists, VEGF-2 receptor antagonists, VEGF-3 receptor antagonists, VEGF-2 receptor modulators, VEGF-B ligand inhibitors, Vimentin inhibitors, VIP 1 receptor agonists, VIP 2 receptor agonists, Vitamin D3 receptor agonists, Vitamin D3 receptor modulators, Vitamin K dependent protein C stimulators, WNT modulators, Wnt ligand inhibitors, Wnt 5A ligand inhibitors, Xanthine oxidase inhibitors, X-linked inhibitor of apoptosis protein inhibitors, XPO1 gene modulators, YAP/TAZ modulators, YSK-4 protein kinase inhibitors, Zap70 tyrosine kinase inhibitors, Zinc finger binding protein Aiolos inhibitors, and zonulin inhibitors.

Rheumatoid Arthritis

In some embodiments, the antibody or antigen-binding fragment thereof of the disclosure is co-administered with one or more agents useful for the treatment and/or prophylaxis of a rheumatological condition.

In some embodiments, the antibody or antigen-binding fragment thereof of the disclosure is co-administered with one or more agents useful for the treatment and/or prophylaxis of rheumatoid arthritis. Non-limiting examples of such agents include disease-modifying antirheumatic drugs (DMARDS), such as hydroxychloroquine, sulfasalazine, methotrexate, and leflunomide; TNF inhibitors (e.g., etanercept, adalimumab, infliximab, golimumab, certolizumab pegol), T cell costimulatory inhibitor, (e.g., abatacept), IL-6 receptor inhibitors (e.g., tocilizumab, sarilumab), anti-CD20 antibody (e.g., rituximab); and JAK inhibitors (e.g., tofacitinib, baricitinib, upadacitinib); NSAIDs, such as ibuprofen, naproxen, and diclofenac; COX-2 inhibitor, such as celecoxib and etoricoxib; steroids and corticosteroids, such as prednisolone and cortisone; and biological agents known for treatment and/or prophylaxis of such conditions, including for example etanercept (e.g., ENBREL), infliximab (e.g., REMICADE), adalimumab (e.g., HUMIRA), anakinra (e.g., KINARET), abatacept (ORENCIA), rituximab (e.g., RITUXAN), certolizumab (e.g., CIMZIA), golimumab (e.g., SIMPONI), and tocilizumab (e.g., ACTEMRA). In some embodiments, a compound of the disclosure is administered with two additional therapeutic agents useful for the treatment and/or prophylaxis of a rheumatological condition. In some embodiments, agents useful for the treatment and/or prophylaxis of a rheumatological condition include a compound of the disclosure and two additional therapeutic agents, such as methotrexate and leflunomide, methotrexate and sulfasalazine, methotrexate and cyclosporine, methotrexate and hydroxychloroquine and triple therapy treatments hydroxychloroquine and sulfasalazine and methotrexate, hydroxychloroquine and sulfasalazine and leflunomide.

Lupus

In some embodiments, a compound of the disclosure, or a pharmaceutically acceptable salt thereof, is co-administered with one or more agents useful for the treatment and/or prophylaxis of systemic lupus erythematosus (SLE) or lupus nephritis (LN). Non-limiting examples of such agents include immunosuppressive drugs that inhibit activity of the immune system and agents approved for treatment of SLE, such as hydroxychloroquine, steroids and corticosteroids (e.g., prednisone, methylprednisolone), belimumab, azathioprine, methotrexate, cyclophosphamide, mycophenolate and mycophenolate mofetil, cyclosporine, leflunomide, voclosporin, abatacept, anifrolumab, rituximab, NSAIDS, such as naproxen sodium and ibuprofen, antimalarial drugs, such as hydroxychloroquine, calcineurin inhibitors, and tacrolimus.

In some embodiments, the antibody or antigen-binding fragment thereof of the disclosure is co-administered with two or more agents useful for the treatment of LN, such as (a) prednisone and mycophenolic acid analogs, (b) prednisone and mycophenolic acid sodium, (c) prednisone and cyclophosphamide, (d) prednisone and tacrolimus, (e) prednisone and voclosporin, (f) prednisone, belimumab and mycophenolic acid analogs, (g) prednisone, belimumab and cyclophosphamide, or (h) prednisone and rituximab.

In further embodiments, a compound of the disclosure, or a pharmaceutically acceptable salt thereof, is co-administered with two or more agents useful for the treatment of LN, such as (a) prednisone and mycophenolic acid analogs, (b) prednisone and mycophenolic acid sodium, (c) prednisone and azathioprine, (d) prednisone and tacrolimus, (e) prednisone and cyclosporine, or (f) prednisone and mizoribine.

Osteoarthritis

In some embodiments, the antibody or antigen-binding fragment thereof of the disclosure is co-administered with one or more agents useful for the treatment and/or prophylaxis of osteoarthritis (OA). Non-limiting examples of such agents include nonsteroidal anti-inflammatory drugs (NSAIDs), topical capsaicin, intraarticular glucocorticoid injections, acetaminophen, duloxetine, tramadol, and injectable corticosteroids such as methylprednisolone acetate, triamcinolone acetate, betamethasone acetate and betamethasone sodium phosphate, triamcinolone hexacetonide, and dexamethasone.

Ulcerative Colitis

In some embodiments, the antibody or antigen-binding fragment thereof of the disclosure is co-administered with one or more agents useful for the treatment and/or prophylaxis of a gastroenterologic condition such as ulcerative colitis (UC) or Crohn's disease (CD). Non-limiting examples of such agents include infliximab, adalimumab, golimumab, vedolizumab, tofacitinib, ustekinumab, natalizumab, mesalamine, diazo-bonded 5-ASA, sulfasalazine, balsalazide, olsalazine, corticosteroids such as budesonide, hydrocortisone, methylprednisolone, and prednisone; immunosuppressants or immunomodulators such as azathioprine and 6-mercaptopurine, cyclosporine, and methotrexate.

Pulmonology

In some embodiments, the antibody or antigen-binding fragment thereof of the disclosure is co-administered with one or more agents useful for the treatment and/or prophylaxis of a pulmonologic condition, such as idiopathic pulmonary fibrosis (IPF) or interstitial lung disease (ILD). Non-limiting examples of such agents include nitendanib, pirfenidone, corticosteroids such as prednisone, other rheumatologic drugs, including mycophenolate (e.g., CellCept®), azathioprine (e.g., Imuran®), leflunomide (e.g., ARAVA®), rituximab (e.g., RITUXAN®), cyclophosphamide (e.g., CYTOXAN®), tacrolimus (e.g., PROGRAF®), medications that reduce stomach acid, such as H-2-receptor antagonists or proton pump inhibitors such as lansoprazole (e.g., PREVACID@24HR), omeprazole (e.g., Prilosec OTC) and pantoprazole (e.g., PROTONIX®).

Heptatology and Nephrology

In some embodiments, the antibody or antigen-binding fragment thereof of the disclosure is co-administered with one or more agents useful for the treatment and/or prophylaxis of a heptatologic or nephrologic condition, such as NAFLD, NASH, DKD, or CKD. Non-limiting examples of such agents include metformin, sodium-glucose cotransporter-2 inhibitor (SGLT2i), drug therapy for glycemic control, DPP-4 inhibitor, insulin, sulfonylurea, TZD (thiazolidinedione), alpha-glucosidase inhibitor, SGLT2 inhibitor (e.g., empagliflozin, canagliflozin, dapaglifloz), glucagon-like peptide-1 receptor agonist (GLP-1 RA) (e.g., lixisenatide, liraglutide, semaglutide, exenatide, albiglutide, dulaglutide), DPP-4 inhibitors (e.g., saxagliptin, alogliptin, sitagliptin, linagliptin), one or more agents used to treat high blood pressure such as angiotensin-converting enzyme (ACE) inhibitors and angiotensin 2 receptor blockers (ARBs), agents supportive of weight loss or for control of blood sugar, cholesterol-lowering drugs (e.g., statins), finerenone, and agents for treatment of diabetes mellitus, such as alpha-glucosidase inhibitors (e.g., acarbose, miglitol, voglibose).

Dermatology

In some embodiments, the antibody or antigen-binding fragment thereof of the disclosure is is co-administered with one or more agents useful for the treatment and/or prophylaxis of a dermatologic condition, such as atopic dermatitis (AD). Non-limiting examples of such agents include topical corticosteroids (TCS) (e.g., desonid, hydrocortisone, fluocinolone, triamcinolone, betamethasone diproprionate), topical calcineurin inhibitors (TCI) (e.g., tacrolimus, pimecrolimus), topical antimicrobials and antiseptics, cyclosporine, methotrexate, mycophenolate mofetil, interferon gamma, phosphodiesterase 4 (PDE4) inhibitor such as crisaborole, JAK inhibitor (e.g., ruxolitinib, upadacitinib, abrocitinib), systemic glucocorticoids (e.g., prednisone), dupilumab, and anti-IL-13 antibody (e.g., tralokinumab)

### EXAMPLES

The following examples are provided to further illustrate some embodiments of the present disclosure but are not intended to limit the scope of the disclosure. It will be understood by their exemplary nature that other procedures, methodologies, or techniques known to those skilled in the art may alternatively be used.

### Example 1. Generation and Sequencing of Anti-CD200R Antibodies

This example illustrates the process of generating anti-CD200R antibodies according to some embodiments of the present disclosure.

Antibodies recognizing the human immune cell receptor CD200R were generated by immunizing mice with the extracellular region of human CD200R. Splenocytes from immunized mice were fused with Sp2/0-Agl4 myeloma cells and resulting hybridomas selected for reactivity with human CD200R by ELISA of supernatants, in conjunction with dilution cloning. Antibodies were isotyped from hybridoma supernatant using a Rapid Mouse Isotyping Kit (RayBiotech). The antibody produced by clone 21.3.1 was found to be IgG1k.

To sequence the immunoglobulin variable domains of selected hybridomas, RNA was extracted from cells using TRIzol Reagent (ThermoFisher) as per the manufacturer's instructions. RNA was reverse transcribed to produce cDNA using primers specific for the first constant domain of the heavy chain or for the constant domain of the light chain, and Super Script II Reverse Transcriptase (Invitrogen) as per manufacturer's instructions.

To amplify immunoglobulin variable domains following RNA and cDNA synthesis, PCR was performed using primers targeting conserved regions of the immunoglobulin locus as previously described (Tiller et al., J Immunol Methods. 350:183-193, 2009) and PCR products were sequenced. In some cases, identification of functional light chain was complicated by abundant non-functional kappa light chain cDNA from the fusion myeloma cell line. To resolve this excess primer specific for the non-functional chain CDR3 was added to force truncation of the aberrant chain product (as described in Yuan et al. J Immunol Methods. 294:39553-61, 2005).

Variable domain sequences were assessed using the NCBI IgBllast tool to identify the closest germline sequences and the CDRs were defined using the Kabat scheme as set forth in Kabat et al. Sequences of Proteins of Immunological Interest, 5th Ed. Public Health Service, National Institutes of Health, Bethesda, MVD. (1991). Selected sequences are shown in Table 1.

TABLE 1
Variable domain and CDR sequences (Kabat) of selected anti-CD200R antibodies.

| Clone | | | SEQ |
|---|---|---|---|
| (mIgG1k | Descrip- | | ID |
| isotype*) | tion | Sequence | NO |
| 21.3.1 | VH | QVQLQQPGSELVRPGASVKLSCKASGHTFTSYWMHWVK | 1 |
| | | QRPGQGLEWIGNIYPGSGSINYDEKFKSKAKLTVDTSSRT | |
| | | AYMQLSSLTSEDSAVYYCLTMTGTSWGQGTLVTVSA | |
| | VL | DVQMIQSPSSLSASLGDIVTMTCQASQGTPINLHWFQQKP | 2 |
| | | GKAPKLLISGASNLEDGVPSRFSGSRYGTAFTLTISSLEDE | |
| | | DMATYFCLQFTYIPWTFGGGTKLEIK | |
| | CDRH1 | SYWMH | 3 |
| | CDRH2 | NIYPGSGSINYDEKFKS | 4 |
| | CDRH3 | MTGTS | 5 |
| | CDRL1 | QASQGTPINLH | 6 |
| | CDRL2 | GASNLED | 7 |
| | CDRL3 | LQFTYIPWT | 8 |
| 26.1.2 | VH | EVQLQQSGPELVKPGASVKMSCKASGYTFTSYVMHWVK | 9 |
| | | QKPGQGLEWIGYIHPYNDDIKHNEKFKDKATLTSAKSSST | |
| | | VYMELSSLTSEDSAVYYCAREEYYGSRFAYWGQGTLVT | |
| | | VSA | |
| | VL | DIVLTQSPASLAVSLGQRATISCRASKSVRTSGYSYLHWY | 10 |
| | | QQKPGQPPKLLIYLASNLESGVPARFSGSGSGTDFTLNIHP | |
| | | VEEEDAATYYCQYSGELPFTFGGGTKLEIK | |
| | CDRH1 | SYVMH | 11 |
| | CDRH2 | YIHPYNDDIKHNEKFKD | 12 |
| | CDRH3 | EEYYGSRFAY | 13 |
| | CDRL1 | RASKSVRTSGYSYLH | 14 |
| | CDRL2 | LASNLES | 15 |
| | CDRL3 | QYSGELPFT | 16 |
| 2.2.7 | VH | QVQLKESGPGLVAPSQSLSITCTVSGFSLTSYGVHWVRQL | 17 |
| | | PGKGLEWLGVIWAGGGTNYNSALMSRLSISKDNSKSQVF | |
| | | LKMNRLQTDDTAIYYCAREGLPRAMDYWGQGTSVTVSS | |
| | VL | DIVLTQSPASLAVSLGQRATISCRASESVDNYGISFMNWF | 18 |
| | | QQKPGQPPKLLSNQGSGVPARFSGSGSGTDFSLNIHPMEE | |
| | | DDTAMYFCQQGKEFPWTFGGGTKLEIN | |
| | CDRH1 | SYGVH | 19 |
| | CDRH2 | VIWAGGGTNYNSALMS | 20 |
| | CDRH3 | EGLPRAMDY | 21 |
| | CDRL1 | RASESVDNYGISFMN | 22 |
| | CDRL2 | QGSGVPA | 23 |
| | CDRL3 | QQGKEFPWT | 24 |
| 3.10.2 | VH | QVQLQQPGSELVRPGASVKLSCKASGYTFTSYWMHWVK | 25 |
| | | QRPGQGLEWIGNIFPGSDTTNYDEKFKSKAIMTVDISSST | |
| | | VYMHLSSLTSEDSAVYYCITYTGAYWGQGTLVTVSA | |
| | VL | DVQMIQSPSSLSASMGDIVTMTCQASQGTNINLHWFQQK | 26 |
| | | PGKAPKLLISGGSNLEDGVPSRFSGSRYGTDFTLTISSLED | |
| | | EDMATYFCLQFTYLPWTFGGGTKLDIK | |
| | CDRH1 | SYWMH | 27 |
| | CDRH2 | NIFPGSDTTNYDEKFKS | 28 |
| | CDRH3 | YTGAY | 29 |
| | CDRL1 | QASQGTNINLH | 30 |
| | CDRL2 | GGSNLED | 31 |
| | CDRL3 | LQFTYLPWT | 32 |
| 7.12.2 | VH | QVQLQQPGSELVRPGASVKLSCKASGYTFTSYWMHWVK | 33 |
| | | QRPGQGLEWIGNIYPGSGTTNYDEKFKSKATLTVDTSS | |
| | | STAYMQISSLTSEDSAVYYCTTGTSTYWGQGTLVTVSA | |
| | VL | DVQMIQSPSSLSASLGDIVTMTCQASQGTSINLNWFQQKP | 34 |
| | | GKAPKLLIYGAVNLEDGVPSRFSGSRYGTDFTLTVSSLE | |
| | | DEDMATYFCLQHTYLPWTFGGGTKLEIK | |
| | CDRH1 | SYWMH | 35 |
| | CDRH2 | NIYPGSGTTNYDEKFKS | 36 |
| | CDRH3 | GTSTY | 37 |
| | CDRL1 | QASQGTSINLN | 38 |
| | CDRL2 | GAVNLED | 39 |
| | CDRL3 | LQHTYLPWT | 40 |

*mIgG1k isotype: heavy chain constant region corresponds to SEQ ID NO: 63 and light chain constant region corresponds to SEQ ID NO: 64

### Example 2. Binding of Murine Antibodies to Soluble Human and Cynomolgus CD200R

This example demonstrates the binding affinity to substrate molecules, human and cynomolgus CD200R, of some anti-CD200R antibodies according to some embodiments of the present disclosure.

The binding affinity and kinetics of the CD200R antibodies of the present disclosure to human or cynomolgus CD200R were determined by surface plasmon resonance (SPR) using the Biacore T200 (GE Healthcare). Mouse antibody capture kit (GE Healthcare) was used to coat a Series S CM5 Sensor Chip (GE Healthcare) with polyclonal anti-mouse IgG.

Anti-CD200R antibody was captured onto the biosensor surface and a negative control antibody (clone Mopc21; Biolegend) captured in the reference channel. Various concentrations of monomeric soluble human CD200R extracellular domain, soluble cynomolgus macaque CD200R1 extracellular domain or soluble cynomolgus macaque CD200RLa (a highly homologous activating receptor) were injected over the immobilized antibodies in the buffer 10 mM Hepes, 150 mM NaCl, 0.005% v/v Surfactant P20, pH 7.4 (HBS-P) at 37° C., in a single cycle kinetics analysis. Association and dissociation rates were fitted using BiaEvaluation Software (GE Healthcare) after reference and blank subtractions, and dissociation constants were calculated (Table 2).

In another experiment, it was also found that antibody 21.3.1 did not bind to cynomolgus CD200RLa expressed on cells, when assessed by flow cytometry.

TABLE 2
Binding affinities of antibodies to human
and cynomolgus CD200R determined by SPR

| Antibody | |
|---|---|
| Clone | Binding K_{D} (nM) |

| (mIgG1k | Human | Cynomolgus | Cynomolgus |
|---|---|---|---|
| isotype)* | CD200R | CD200R | CD200RLa |
| 21.3.1 | 0.3 | 0.00825 | No binding |
| 2.2.7 | 27.6 | 159 | (injected at |
| 3.10.2 | 0.4 | 0.0015 | up to 2 μM) |
| 7.12.2 | 2.9 | 0.708 | |
| 26.1.2 | 0.7 | 60 | |

*mIgG1k isotype: heavy chain constant region corresponds to SEQ ID NO: 63 and light chain constant region corresponds to SEQ ID NO: 64

### Example 3. Competition with the Natural Ligand CD200 for Binding to CD200R

This example demonstrates that some anti-CD200R antibodies according to some embodiments of the present disclosure have no competition with the natural ligand CD200 for binding to CD200R.

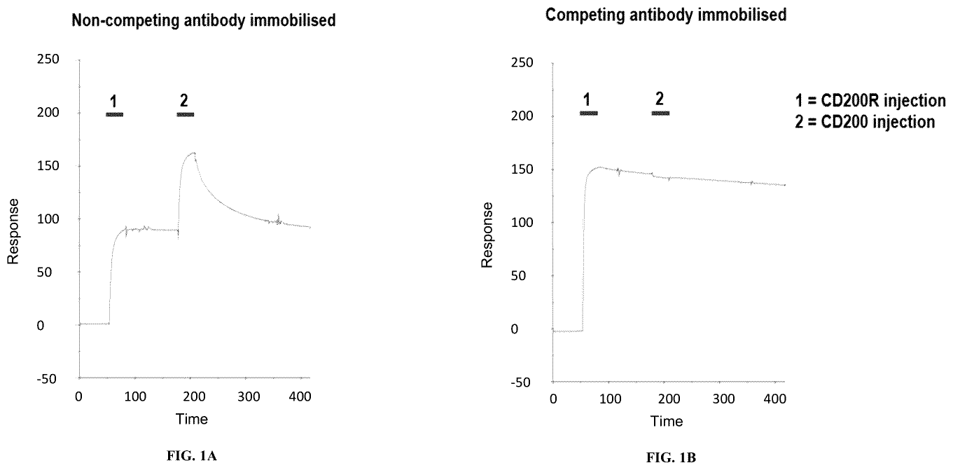

The ability of the CD200R antibodies of the present disclosure to compete with natural ligand binding to CD200R was assessed by surface plasmon resonance using the Biacore 3000 (GE Healthcare). Mouse antibody capture kit (GE Healthcare) was used to coat a CM5 Sensor Chip (GE Healthcare) with polyclonal anti-mouse IgG. Anti-CD200R antibody was captured onto the biosensor surface and a negative control antibody (clone Mopc21; Biolegend) captured in the reference channel. Human CD200R extracellular domain was injected over the immobilized antibodies in the buffer 10 mM Hepes, 150 mM NaCl, 0.005% v/v Surfactant P20, pH 7.4 (HBS-P) at 25° C., followed immediately by an injection of human CD200. If CD200 is able to bind to the captured CD200R then the binding epitope for that antibody must be non-competing with CD200's binding site (exemplified in FIG. 1A), whereas inability of CD200 to bind the captured CD200R demonstrates a competing epitope (FIG. 1B). Using this method, the following antibodies were found to bind non-competing epitope.

TABLE 3
Summary of non-competing and competing anti-CD200R clones

| non-competing clones | competing clones |
|---|---|
| (mIgG1k isotype*) | (mIgG1k isotype*) |
| 21.3.1 | 26.1.2 |
| 2.2.7 | |
| 3.10.2 | |
| 7.12.2 | |

*mIgG1k isotype: heavy chain constant region corresponds to SEQ ID NO: 63 and light chain constant region corresponds to SEQ ID NO: 64

### Example 4. Binding Epitope of Antibodies on Human CD200R

This example characterizes the binding epitope(s) of some anti-CD200R antibodies according to some embodiments of the present disclosure.

The functional epitope of antibodies on human CD200R was determined by flow cytometry assessment of binding to a panel of single residue mutants of the receptor expressed on the cell surface. Constructs encoding the human extracellular region of CD200R with the transmembrane and intracellular regions of murine CD28 were cloned into the bi-cistronic mammalian expression vector pGFP2-n2 (BioSignal Packard Ltd), which also encodes GFP. Mutant constructs varying by one amino acid were prepared using the “drastic” mutagenesis approach (Davis et al. Proc Natl Acad Sci USA. 95, 5490-4 (1998)).

Plasmids (2 pg/well) were transfected into HEK-293T cells in 6 well plates using Genejuice transfection reagent (Novagen; 6 μl/well). Mock and no-transfection controls were included with each experiment. Cells were harvested at 48 hours and stained with anti-CD200R antibody at 10 pg/ml, alongside a Live/Dead marker, in PBS, 0.05% azide, 2% FCS (FACS buffer) for 1 hour at 4° C., washed, and stained with an AF647 conjugated anti-mIgG secondary antibody. Cells were washed, pelleted and resuspended in 200 μl FACS buffer before being analyzed on a BD FACSCanto flow cytometer. GFP-positive (transfected) viable cells were gated and analyzed for binding of anti-CD200R antibodies.

For each mutant, the Geo-mean of anti-CD200R antibody binding to transfected cells was calculated as a percentage of binding to the wild-type receptor. A panel of anti-CD200R antibodies was assessed and any mutation that eliminated binding of all antibodies was excluded from the analysis, on the assumption that such mutations lead to drastic changes in protein folding or expression rather than indicating an antibody epitope.

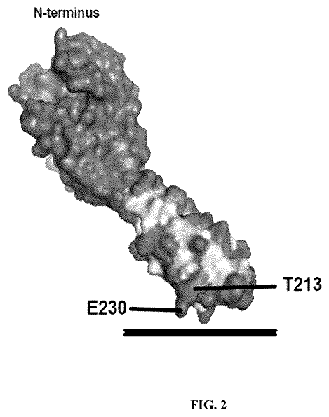

Mutation of residues T213 and E230 on CD200R abolished binding of antibody 21.3.1. These mutations were mapped onto a homology model of the human CD200R structure (PDB Q8TD46 from the SWISS-MODEL repository) (FIG. 2) indicating the binding epitope of 21.31. The epitope was found to be near the C-terminus of CD200R which was close to the likely position of the cell membrane. Of note, position 230 is a glutamic acid (a negatively charged residue) in both the human and cyno CD200R sequences but is a lysine (a positively charged residue) in the sequences of the activating homolog receptors human and cyno CD200RLa (also known as CD200R1L). This difference in charge at position 230 may provide a molecular explanation for the binding selectivity of antibody 21.3.1 to the inhibitory receptors.

Conversely, mutation at residue S194 abolished binding of antibody clone 2.2.7 suggesting an epitope close to the N-terminus of domain 2, more distal from the cell membrane.

### Example 5. Antibody 21.3.1 Exhibits Agonistic Activity in a Monocyte Reporter Assay

This example demonstrates that exemplary CD200R antibody, 21.3.1, according to some embodiments of the present disclosure, has improved agonistic activity as compared to other reported CD200R antibody.

The abilities of antibody 21.3.1 and a reported CD200R agonist antibody, I-4P of WO2020055943A1, to inhibit NF-KB signalling were assessed using a human monocyte reporter cell line, THP-1 dual (Invivogen). VH, VL, and CDR sequences of I-4P are shown in Table 5 below. These cells express an NF-κB-inducible secreted alkaline phosphatase (SEAP) reporter gene and were stably transduced with cDNA for full-length human CD200R using a lentiviral system. CD200R-expressing THP-1 dual cells were seeded in a 96-U-bottom plate (4×10⁴ cells/well) and pre-incubated for 1 hour at 37° C. with various concentrations of CD200R antibody or relevant isotype control. Without washing, cells were transferred to a 96-F-bottom plate coated with human IgG1 isotype control (Biolegend) for stimulation via their FcγRs.

After 20 hours of incubation at 37C, cell supernatants were harvested and SLAP activity, corresponding to NF-KB pathway activation, determined by Quanti-BLUE assay (Invivogen). Percentage inhibition of SEAP activity was calculated relative to plate bound IgG stimulation only. As shown in Table 4, antibodies 21.3.1 and J-4P inhibited IgG1-mediated activation of CD200R-expressing THP-1 dual cells. Average IC₅₀ values (calculated using a four-parameter logistic curve) were 0.141 nM and 0.134 nM, respectively. The average maximal percentage inhibition (Eₘₐₓ) by antibody 21.3.1 was 3324, compared to 19.90 by antibody 54P.

Cells collected following essentially the same experimental setup as described above will be subjected to RNA sequencing. Agonist clone 21.3.1 can lead to significant downregulation of more inflammatory genes than antibody J-4P including, for example, genes in the pathway of TNF alpha signaling via NFκB.

TABLE 4
Inhibition of NF-κB signalling by CD200R antibodies 21.3.1 and I-4P

| | Isotype control of | | | | Isotype control of | | | |
|---|---|---|---|---|---|---|---|---|
| | Antibody 21.3.1 | | Antibody 21.3.1 | | Antibody I-4P | | Antibody I-4P | |
| | Average % | | Average % | | Average % | | Average % | |
| Log | inhibition | | inhibition | | inhibition | | inhibition | |
| [Antibody], | of SEAP | | of SEAP | | of SEAP | | of SEAP | |
| M | activity | SD | activity | SD | activity | SD | activity | SD |
| −8.18 | −2.9 | 1.8 | 35.3 | 7.4 | 5.0 | 12.7 | 22.3 | 5.9 |
| −8.8 | −4.8 | 2.9 | 28.3 | 7.1 | 2.1 | 10.9 | 20.9 | 5.2 |
| −9.57 | −6.3 | 3.5 | 23.4 | 8.3 | 1.2 | 10.4 | 16.1 | 6.8 |
| −10.27 | −6.6 | 3.5 | 9.8 | 3.8 | 0.2 | 11.0 | 7.7 | 4.4 |
| −10.9 | −6.8 | 4.2 | 1.1 | 5.0 | −3.0 | 11.6 | 3.2 | 2.8 |
| −11.6 | −5.4 | 1.1 | 1.0 | 5.4 | −5.6 | 9.8 | 2.2 | 1.4 |
| −12.3 | −2.9 | 3.5 | 1.7 | 2.4 | −0.6 | 5.5 | 3.1 | 3.7 |

% inhibition calculated relative to plate-bound IgG1 stimulation only.
n = 4 independent experiments

TABLE 5
Amino acid sequences of Antibody I-4P

| Antibody | SEQ ID | |
|---|---|---|
| I-4P | NO | Amino acid sequence |
| VH | 53 | QVQLVQSGAEVKKPGASVKVSCKASGFSFSSGYYMAWVRQAP |
| | | GQGLEWMGLIGVGSGSLWYAQKFQGRVTMTRDTSTSTVYMEL |
| | | SSLRSEDTAVYYCARHFALSDPFNLWGQGTLVTVSS |
| VL | 54 | EIVLTQSPDFQSVTPKEKVTITCQASESIDSYLLWYQQKPDQSPK |
| | | LLIKQASTLASGVPSRFSGSGSGTDFTLTINSLEAEDAATYYCQN |
| | | YYDISSNDFGGGTKVEIK |
| CDRH1 | 55 | SGYYMA |
| CDRH2 | 56 | LIGVGSGSLWYAQKFQG |
| CDRH3 | 57 | HFALSDPFNL |
| CDRL1 | 58 | QASESIDSYLL |
| CDRL2 | 59 | QASTLAS |
| CDRL3 | 60 | QNYYDISSND |
| HC (full | 73 | QVQLVQSGAEVKKPGASVKVSCKASGFSFSSGYYMAWVRQAP |
| length) | | GQGLEWMGLIGVGSGSLWYAQKFQGRVTMTRDTSTSTVYMEL |
| | | SSLRSEDTAVYYCARHFALSDPFNLWGQGTLVTVSSASTKGPSV |
| | | FPLAPCSRSTSESTAALGCLVKDYFPEPVTVSWNSGALTSGVHTF |
| | | PAVLQSSGLYSLSSVVTVPSSSLGTKTYTCNVDHKPSNTKVDKR |
| | | VESKYGPPCPPCPAPEFLGGPSVFLFPPKPKDTLMISRTPEVTCVV |
| | | VDVSQEDPEVQFNWYVDGVEVHNAKTKPREEQFNSTYRVVSV |
| | | LTVLHQDWLNGKEYKCKVSNKGLPSSIEKTISKAKGQPREPQVY |
| | | TLPPSQEEMTKNQVSLTCLVKGFYPSDIAVEWESNGQPENNYKT |
| | | TPPVLDSDGSFFLYSRLTVDKSRWQEGNVFSCSVMHEALHNHY |
| | | TQKSLSLSLG |
| LC (full | 74 | EIVLTQSPDFQSVTPKEKVTITCQASESIDSYLLWYQQKPDQSPK |
| length) | | LLIKQASTLASGVPSRFSGSGSGTDFTLTINSLEAEDAATYYCQN |
| | | YYDISSNDFGGGTKVEIKRTVAAPSVFIFPPSDEQLKSGTASVVC |
| | | LLNNFYPREAKVQWKVDNALQSGNSQESVTEQDSKDSTYSLSS |
| | | TLTLSKADYEKHKVYACEVTHQGLSSPVTKSFNRGEC |

### Example 6. Humanization of Antibody Clone 21.3.1

This example illustrates the generation of humanized anti-CD200R antibodies according to some embodiments of the present disclosure.

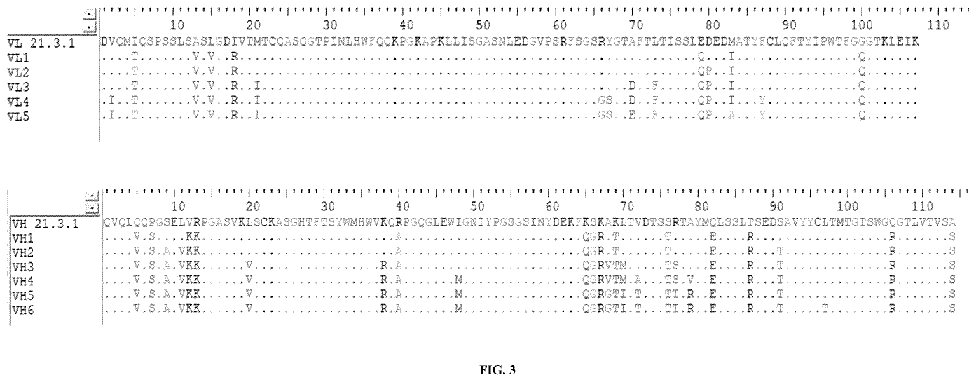

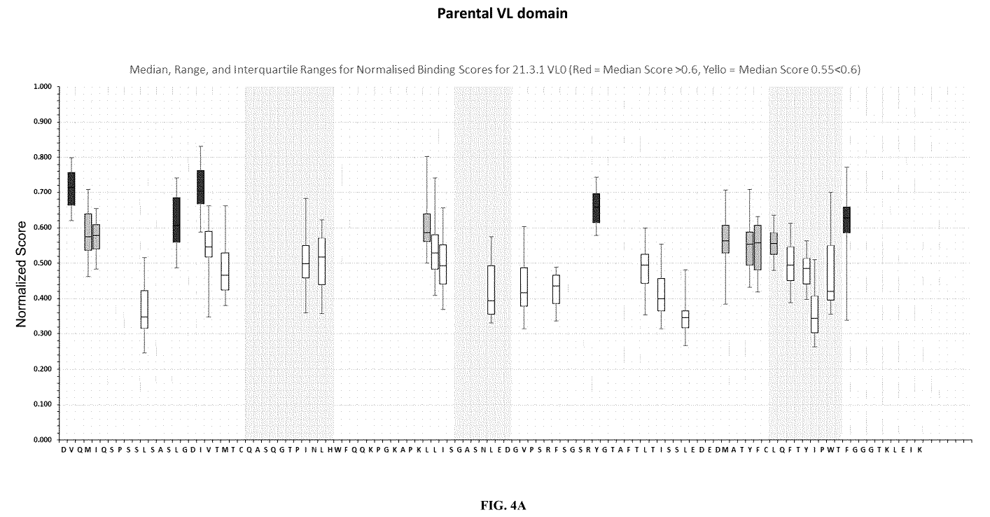

The variable domains of antibody clone 21.3.1 were humanized by germlining to homologous human germline framework regions. Six heavy chain variants and five light chain variants were produced with varying numbers of substitutions to bring them closer to human germline sequence. The CDRs were not changed by humanization except for CDRHI2 which was altered by 2 amino acids from NIYPGSGSINYDEKFKS (SEQ TD NO: 4) to NIYPGSGSINYDEKFQG (SEQ ID NO: 41) in all humanized heavy chain variants. The sequences of the humanized variable domains are outlined in Table 6 and their alignment to the murine parent sequences is shown in FIG. 3.

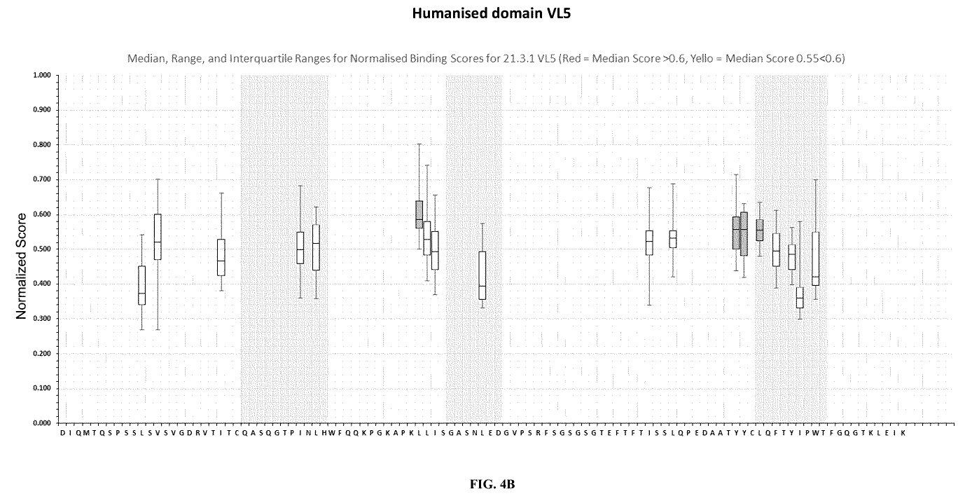

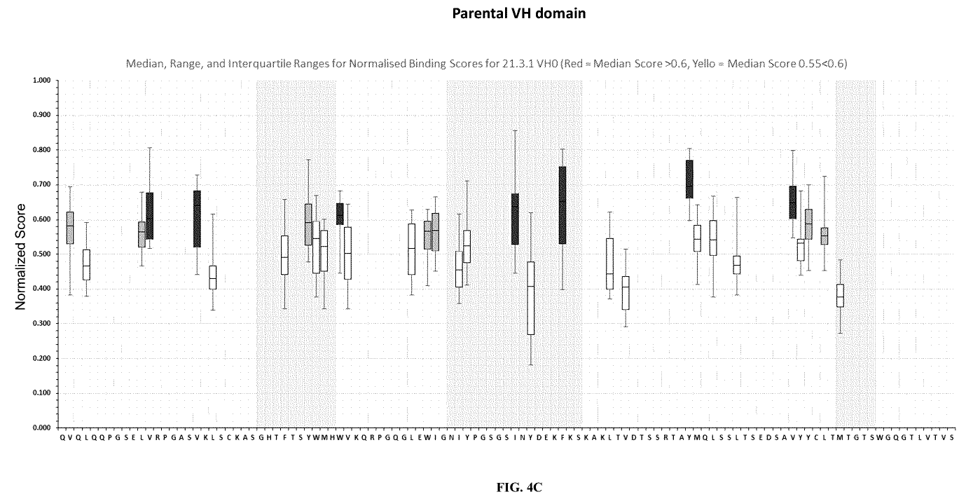

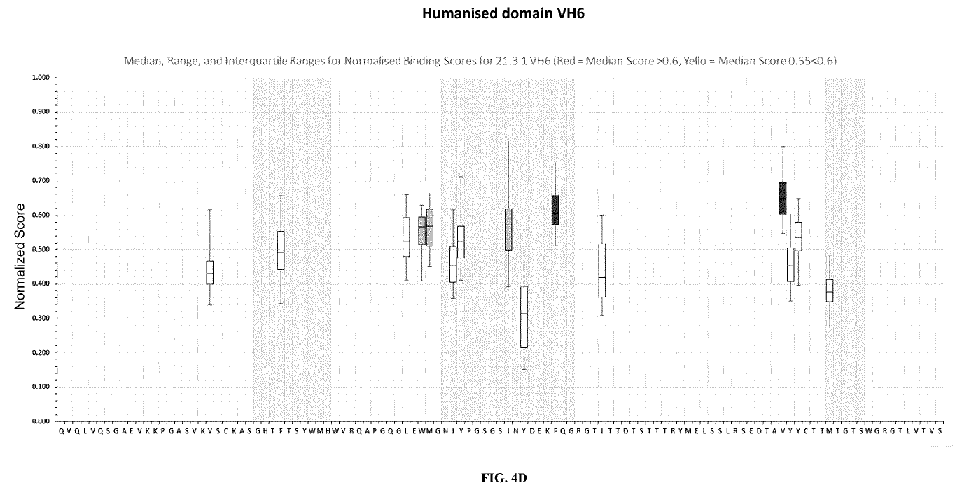

An MHC class II binding assessment was used to calculate the binding scores for all possible monomer peptides within each sequence to 52 HLA-DR alleles. FIGS. 4A-4D show the predicted binding scores for the parent variable domains and the most humanized heavy and light chain variant. The Y axis of each chart displays the normalized binding scores (0-1) across all alleles with the first amino acid of each monomer peptide on the X axis. The box-and-whiskers format shows the median, interquartile range, and the total range of all binding scores for all alleles. Humanization of antibody 21.3.1 led to a reduction in potential MHC II binding peptides, while avoiding significant changes in the CDR loops to avoid disruption of the paratope binding interface.

The CDRL2 loop was noted to contain an aspartate residue with isomerization risk (position D56). Additional light chain variable variants were produced substituting this residue for either a serine or threonine.

TABLE 6
Sequences of humanized VH and VL domains for antibody clone 21.3.1

| | Sequence | SEQ ID NO |
|---|---|---|
| VH | QVQLQQPGSELVRPGASVKLSCKASGHTFTSYWMHWVKQRP | 1 |
| 21.3.1 | GQGLEWIGNIYPGSGSINYDEKFKSKAKLTVDTSSRTAYMQLS | |
| parental | SLTSEDSAVYYCLTMTGTSWGQGTLVTVSA | |
| VH1 | QVQLVQSGSELKKPGASVKLSCKASGHTFTSYWMHWVKQAP | 42 |
| | GQGLEWIGNIYPGSGSINYDEKFQGRATLTVDTSTRTAYMELS | |
| | SLRSEDSAVYYCLTMTGTSWGRGTLVTVSS | |
| VH2 | QVQLVQSGAEVKKPGASVKLSCKASGHTFTSYWMHWVKQA | 43 |
| | PGQGLEWIGNIYPGSGSINYDEKFQGRATLTVDTSTRTAYMEL | |
| | SSLRSEDTAVYYCLTMTGTSWGRGTLVTVSS | |
| VH3 | QVQLVQSGAEVKKPGASVKVSCKASGHTFTSYWMHWVRQA | 44 |
| | PGQGLEWIGNIYPGSGSINYDEKFQGRVTMTVDTSTSTAYME | |
| | LSSLRSEDTAVYYCLTMTGTSWGRGTLVTVSS | |
| VH4 | QVQLVQSGAEVKKPGASVKVSCKASGHTFTSYWMHWVRQA | 45 |
| | PGQGLEWMGNIYPGSGSINYDEKFQGRVTMTADTSTSTVYME | |
| | LSSLRSEDTAVYYCLTMTGTSWGRGTLVTVSS | |
| VH5 | QVQLVQSGAEVKKPGASVKVSCKASGHTFTSYWMHWVRQA | 46 |
| | PGQGLEWMGNIYPGSGSINYDEKFQGRGTITTDTSTTTRYMEL | |
| | SSLRSEDTAVYYCLTMTGTSWGRGTLVTVSS | |
| VH6 | QVQLVQSGAEVKKPGASVKVSCKASGHTFTSYWMHWVRQA | 47 |
| | PGQGLEWMGNIYPGSGSINYDEKFQGRGTITTDTSTTTRYMEL | |
| | SSLRSEDTAVYYCTTMTGTSWGRGTLVTVSS | |
| VL | DVQMIQSPSSLSASLGDIVTMTCQASQGTPINLHWFQQKPGKA | 2 |
| 21.3.1 | PKLLISGASNLEDGVPSRFSGSRYGTAFTLTISSLEDEDMATYF | |
| parental | CLQFTYIPWTFGGGTKLEIK | |
| VL1 | DVQMTQSPSSLSVSVGDRVTMTCQASQGTPINLHWFQQKPGK | 48 |
| | APKLLISGASNLEDGVPSRFSGSRYGTAFTLTISSLQDEDIATYF | |
| | CLQFTYIPWTFGQGTKLEIK | |
| VL2 | DVQMTQSPSSLSVSVGDRVTMTCQASQGTPINLHWFQQKPGK | 49 |
| | APKLLISGASNLEDGVPSRFSGSRYGTAFTLTISSLQPEDIATYF | |
| | CLQFTYIPWTFGQGTKLEIK | |
| VL3 | DVQMTQSPSSLSVSVGDRVTITCQASQGTPINLHWFQQKPGK | 50 |
| | APKLLISGASNLEDGVPSRFSGSRYGTDFTFTISSLQPEDIATYF | |
| | CLQFTYIPWTFGQGTKLEIK | |
| VL4 | DIQMTQSPSSLSVSVGDRVTITCQASQGTPINLHWFQQKPGKA | 51 |
| | PKLLISGASNLEDGVPSRFSGSGSGTDFTFTISSLQPEDIATYYC | |
| | LQFTYIPWTFGQGTKLEIK | |
| VL5 | DIQMTQSPSSLSVSVGDRVTITCQASQGTPINLHWFQQKPGKA | 52 |
| | PKLLISGASNLEDGVPSRFSGSGSGTEFTFTISSLQPEDAATYYC | |
| | LQFTYIPWTFGQGTKLEIK | |
| VL5- | DIQMTQSPSSLSVSVGDRVTITCQASQGTPINLHWFQQKPGKA | 65 |
| D56S | PKLLISGASNLESGVPSRFSGSGSGTEFTFTISSLQPEDAATYYC | |
| | LQFTYIPWTFGQGTKLEIK | |
| VL5- | DIQMTQSPSSLSVSVGDRVTITCQASQGTPINLHWFQQKPGKA | 66 |
| D56T | PKLLISGASNLETGVPSRFSGSGSGTEFTFTISSLQPEDAATYYC | |
| | LQFTYIPWTFGQGTKLEIK | |

### Example 7: Assessment of Humanized Antibodies Binding to Soluble CD200R

Humanised variant antibodies combining the different heavy and light variable domains described in Example 6 were produced by transient transfection of HEK293 cells. All antibodies were expressed on a human IgGI isotype with a P238D Fc mutation. The unpurified supernatants were analysed by Protein A-HPLC to determine the antibody concentrations, as a measure of expression yield. Antibodies containing humanised VH5 or VH6 variable domains failed to express and were not further assessed. Binding affinity of humanized variants to human CD200R was assessed by bio-layer interferometry on an Octet R4 instrument. Each antibody was captured directly onto anti-human Fc capture probes (Sartorius 18-5060) for 60 sec followed by baseline setting in assay buffer (DPBS, 0.01% Polysorbate-20). Association of monomeric CD200R protein to immobilised antibodies was measured in 4 different protein concentrations (300, 100, 30 and 10 nM in assay buffer) at 25° C., with an association time of 120s and dissociation of 120s in assay buffer only. A reference probe with no antibody immobilised was used for background subtraction. Kinetic rate constants and binding KDs were calculated using Octet Analysis Studio 12 software. Global curve fitting based on all 4 concentrations using a 1:1 model was performed, from which kon and koff were obtained to calculate KD using the relationship KD=koff/kon.

A chimeric antibody containing the murine 21.3.1 variable domains and a human IgG1 Fc with P238D mutation (2131_chimera) was included as a control. Additionally, the prior art anti-CD200R agonist antibody I-4P (WO2020055943A1) was assessed alongside.

All humanized variants of 2131 assessed demonstrated very high affinity binding to human CD200R with KDs ranging from 1 μM to 0.18 nM (Table 7). Antibody I-4P demonstrated a binding KD at 25° C. of 2.16 nM, in keeping with the binding KD of 5.6 nM at 37° C. described in WO2020055943A1.

TABLE 7
Expression yields and binding affinity
of humanized variants of 21.3.1

| | | | concentration | hCD200R KD |
|---|---|---|---|---|
| Variant | VH | VL | (ug/ml) | (M) |
| hu2131_v1 | VH1 | VL1 | 43 | 3.629E−12 |
| hu2131_v2 | VH1 | VL2 | 47 | 3.844E−12 |
| hu2131_v3 | VH1 | VL3 | 42 | 3.876E−12 |
| hu2131_v4 | VH1 | VL4 | 42 | 2.736E−12 |
| hu2131_v5 | VH1 | VL5 | 45 | 5.003E−12 |
| hu2131_v6 | VH2 | VL1 | 45 | 3.965E−12 |
| hu2131_v7 | VH2 | VL2 | 51 | 3.058E−12 |
| hu2131_v8 | VH2 | VL3 | 45 | 3.184E−12 |
| hu2131_v9 | VH2 | VL4 | 44 | 3.070E−12 |
| hu2131_v10 | VH2 | VL5 | 46 | 4.426E−12 |
| hu2131_v11 | VH3 | VL1 | 58 | 2.453E−12 |
| hu2131_v12 | VH3 | VL2 | 61 | 4.141E−12 |
| hu2131_v13 | VH3 | VL3 | 53 | 2.941E−12 |
| hu2131_v14 | VH3 | VL4 | 52 | 2.900E−12 |
| hu2131_v15 | VH3 | VL5 | 54 | 3.183E−12 |
| hu2131_v16 | VH4 | VL1 | 47 | 1.031E−12 |
| hu2131_v17 | VH4 | VL2 | 51 | 1.831E−10 |
| hu2131_v18 | VH4 | VL3 | 47 | 3.436E−11 |
| hu2131_v19 | VH4 | VL4 | 45 | 4.230E−12 |
| hu2131_v20 | VH4 | VL5 | 47 | 2.755E−12 |
| hu2131_v22 | VH5 | VL2 | nd | Not tested |
| hu2131_v23 | VH5 | VL3 | nd | Not tested |
| hu2131_v24 | VH5 | VL4 | nd | Not tested |
| hu2131_v25 | VH5 | VL5 | nd | Not tested |
| hu2131_v27 | VH6 | VL2 | nd | Not tested |
| hu2131_v28 | VH6 | VL3 | nd | Not tested |
| hu2131_v29 | VH6 | VL4 | nd | Not tested |
| hu2131_v30 | VH6 | VL5 | nd | Not tested |
| hu2131_v20_D56S | VH4 | VL5- | NA (purified | 2.06E−12 |
| | | D56S | for analysis) | |
| 2131_chimera | WT | WT | 26 | 3.042E−12 |
| Antibody I-4P | — | — | NA | 2.16E−09 |

### Example 8: Generation of Additional Variants of Humanized Antibody Clone 21.3.1

In this example, additional mutations were introduced to the humanized antibody hu2131_v20_D56S (which comprised (a) the variable heavy chain sequence of VH4, corresponding to SEQ ID NO: 45, linked to the IgGI P238D constant region, corresponding to SEQ ID NO: 6i) and (b) the variable light chain sequence of VL5-D56S, corresponding to SEQ ID NO: 65, linked to the Kappa constant region, corresponding to SEQ ID NO: 62), to produce variants of a humanized antibody of clone 21.3.1. Specifically, a single residue mutation of CDRHI3 was introduced to overcome potential oxidation liabilities. CDRHI3 was changed from MTGTS (SEQ ID NO: 5) to GTGTS (SEQ ID NO: 70). In addition, VH Q1 was mutated to El to avoid heterogeneity due to pyroglutamate formation. The sequences of the resulting variants, which are referred to as antibody hu2131_Q1E and antibody hu2131_Q1E_M99G, are shown in Tables 8 and 9, respectively. The full-length sequences of hu2131_v20_D56S hIgG1k P238D isotype, hu213l_Q1E hIgG1k P238D isotype and antibody hu2131_Q1E_M99G hIgG1k P238D isotype are shown in Table 10. As shown in Table 10, the full-length light chain (LC) sequence for all three antibodies corresponds to the amino acid sequence of SEQ ID NO. 86.

TABLE 8
Antibody hu2131_Q1E Sequences

| | SEQ ID | |
|---|---|---|
| Description | NO | Amino acid sequence |
| VH | 71 | EVQLVQSGAEVKKPGASVKVSCKASGHTFTSYWMHWVRQA |
| | | PGQGLEWMGNIYPGSGSINYDEKFQGRVTMTADTSTSTVYM |
| | | ELSSLRSEDTAVYYCLTMTGTSWGRGTLVTVSS |
| VL | 65 | DIQMTQSPSSLSVSVGDRVTITCQASQGTPINLHWFQQKPGKA |
| | | PKLLISGASNLESGVPSRFSGSGSGTEFTFTISSLQPEDAA |
| | | TYYCLQFTYIPWTFGQGTKLEIK |
| CDRH1 | 3 | SYWMH |
| CDRH2 | 41 | NIYPGSGSINYDEKFQG |
| CDRH3 | 5 | MTGTS |
| CDRL1 | 6 | QASQGTPINLH |
| CDRL2 | 67 | GASNLES |
| CDRL3 | 8 | LQFTYIPWT |

TABLE 9
hu2131_Q1E M99G Sequences

| | SEQ ID | |
|---|---|---|
| Description | NO | Amino acid sequence |
| VH | 72 | EVQLVQSGAEVKKPGASVKVSCKASGHTFTSYWMHWVR |
| | | QAPGQGLEWMGNIYPGSGSINYDEKFQGRVTMTADTSTST |
| | | VYMELSSLRSEDTAVYYCLTGTGTSWGRGTLVTVSS |
| VL | 65 | DIQMTQSPSSLSVSVGDRVTITCQASQGTPINLHWFQQKPG |
| | | KAPKLLISGASNLESGVPSRFSGSGSGTEFTFTISSLQPEDAA |
| | | TYYCLQFTYIPWTFGQGTKLEIK |
| CDRH1 | 3 | SYWMH |
| CDRH2 | 41 | NIYPGSGSINYDEKFQG |
| CDRH3 | 70 | GTGTS |
| CDRL1 | 6 | QASQGTPINLH |
| CDRL2 | 67 | GASNLES |
| CDRL3 | 8 | LQFTYIPWT |

TABLE 10
Full Length Heavy Chain (HC) and Light Chain (LC) Sequences

| | SEQ ID | |
|---|---|---|
| Description | NO | Amino acid sequence |
| Antibody | 79 | QVQLVQSGAEVKKPGASVKVSCKASGHTFTSYWMHWVR |
| hu2131_v20_D56S | | QAPGQGLEWMGNIYPGSGSINYDEKFQGRVTMTADTSTST |
| hIgG1k P238D HC- | | VYMELSSLRSEDTAVYYCLTMTGTSWGRGTLVTVSSASTK |
| full length | | GPSVFPLAPSSKSTSGGTAALGCLVKDYFPEPVTVSWNSGA |
| | | LTSGVHTFPAVLQSSGLYSLSSVVTVPSSSLGTQTYICNVNH |
| | | KPSNTKVDKKVEPKSCDKTHTCPPCPAPELLGGDSVFLFPP |
| | | KPKDTLMISRTPEVTCVVVDVSHEDPEVKFNWYVDGVEV |
| | | HNAKTKPREEQYNSTYRVVSVLTVLHQDWLNGKEYKCKV |
| | | SNKALPAPIEKTISKAKGQPREPQVYTLPPSREEMTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSP |
| | | GK |
| Antibody | 80 | EVQLVQSGAEVKKPGASVKVSCKASGHTFTSYWMHWVR |
| hu2131_Q1E- | | QAPGQGLEWMGNIYPGSGSINYDEKFQGRVTMTADTSTST |
| hIgG1k P238D HC- | | VYMELSSLRSEDTAVYYCLTMTGTSWGRGTLVTVSSASTK |
| full length | | GPSVFPLAPSSKSTSGGTAALGCLVKDYFPEPVTVSWNSGA |
| | | LTSGVHTFPAVLQSSGLYSLSSVVTVPSSSLGTQTYICNVNH |
| | | KPSNTKVDKKVEPKSCDKTHTCPPCPAPELLGGDSVFLFPP |
| | | KPKDTLMISRTPEVTCVVVDVSHEDPEVKFNWYVDGVEV |
| | | HNAKTKPREEQYNSTYRVVSVLTVLHQDWLNGKEYKCKV |
| | | SNKALPAPIEKTISKAKGQPREPQVYTLPPSREEMTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSP |
| | | GK |
| hu2131_Q1E_M99G | 81 | EVQLVQSGAEVKKPGASVKVSCKASGHTFTSYWMHWVR |
| hIgG1k P238D HC- | | QAPGQGLEWMGNIYPGSGSINYDEKFQGRVTMTADTSTST |
| full length | | VYMELSSLRSEDTAVYYCLTGTGTSWGRGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLGGDSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSREEMTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPGK |
| LC-full length | 86 | DIQMTQSPSSLSVSVGDRVTITCQASQGTPINLHWFQQKPG |
| | | KAPKLLISGASNLESGVPSRFSGSGSGTEFTFTISSLQPEDAA |
| | | TYYCLQFTYIPWTFGQGTKLEIKRTVAAPSVFIFPPSDEQLK |
| | | SGTASVVCLLNNFYPREAKVQWKVDNALQSGNSQESVTE |
| | | QDSKDSTYSLSSTLTLSKADYEKHKVYACEVTHQGLSSPVT |
| | | KSFNRGEC |

### Example 9: Assessment of Variant Humanized Antibodies Binding to Soluble CD20OR

This example investigates in vitro CD200R binding of anti-CD200R antibodies by surface plasmon resonance. Surface plasmon resonance binding experiments with purified anti-CD200R antibody (expressed on a hIgG1 isotype with a P238D Fc mutation) were performed using the Biacore T200 and 8K+instruments. Anti-human Fc antibody (Life Technologies H10500) was amine-coupled to a C1 sensor chip using standard amine-coupling chemistry. Residual activated carboxyls were blocked with ethanolamine. Antibodies with a human Fc were then captured on these surfaces.

To assess the binding of hu2131_v20_D56S antibody and variants (antibody hu213l_Q1E and antibody hu2131_Q1E_M99G) to the extracellular domains of human CD200R (Met29-Leu243) and cyno CD200R (SinoBiological 11232-C08H, Metl-Leu267), 10-30 RUs of antibody were captured. CD200R proteins were diluted into running buffer (10 mM HEPES, 150 mM NaCl, 3 mM EDTA, 0.01% Polysorbate 20, pH 7.4 with 0.1 mg/mL BSA) (full-length antibody sequences are disclosed in Table 10). Five three-fold serial dilutions were performed for each CD200R, with highest concentration being 100 nM. Samples were injected using a single cycle kinetics scheme (Karlsson Anal Biochem et al 2006). Data were analyzed using Biacore Insight Evaluation Software and fit to a simple kinetic model, from which kon and koff were obtained to calculate KD using the relationship KD=koff/kon. Experiments were run at 25° C. The CD200R binding profiles of the anti-CD200R antibodies are shown in Table 11.

TABLE 11
CD200R binding profiles for 21.3.1 antibody and variants

Antibody Clone

| (hIgG1k P238D | Human CD200R | | | Cyno CD200R | | |
|---|---|---|---|---|---|---|
| isotype) | K_{D} (nM) | kₒₙ (M⁻¹*s⁻¹) | k_{off} (s⁻¹) | K_{D} (nM) | kₒₙ (M⁻¹*s⁻¹) | k_{off} (s⁻¹) |
| hu2131_v20_D56S | ≤0.0166 | 6.02 × 10⁵ | ≤1 × 10⁻⁵ | 0.041 | 2.58 × 10⁵ | 1.06 × 10⁻⁵ |
| hu2131_Q1E | ≤0.0168 | 5.97 × 10⁵ | ≤1 × 10⁻⁵ | ≤0.0392 | 2.55 × 10⁵ | ≤1 × 10⁻⁵ |
| hu2131_Q1E_M99G | ≤0.0193 | 5.17 × 10⁵ | ≤1 × 10⁻⁵ | 0.0917 | 2.44 × 10⁵ | 2.23 × 10⁻⁵ |

Some reported K_{D} values are expressed as an inequality since koff measurements could not be accurately determined. In these instances, values for koff were fixed at 1x10-⁵ s⁻¹. As shown in Table 11, the single and double mutant variants produced (antibody hu2131_Q1E and antibody hu2131_Q1E_M99G respectively) showed similar binding to the target and potency as hu2131_v20_D56S.

### Example 10: Cell Surface CD200R Binding Potency by Flow Cytometry

This example investigates binding of anti-CD200R antibodies to cell surface expressed human CD200R by flow cytometry. A Jurkat T cell line lentivirally infected to express human CD200R was used. 2×10⁴ cells per well were plated in 96 well U-bottom plates. CD200R antibodies hu2131_Q1E_M99G or AbI-4P or the respective isotype controls were assessed at 8 concentrations by 1 in 5 serial dilution in tissue culture media (RPMI, 10% FCS), starting at a concentration of 25 μg/ml. Antibodies were incubated with cells for 24 hours at 37C, 5% CO2. Cells were then washed twice with FACS buffer (PBS 1% FCS, 0.05% sodium azide) prior to staining with a PE conjugated anti-hIgG secondary antibody (Biolegend #366904). Secondary antibody was incubated for 30 minutes on ice, then cells were washed and resuspended in FACS buffer for analysis on a flow cytometer. The geometric mean fluorescent intensity of secondary antibody was plotted for each concentration and the EC50 for receptor binding calculated by non-linear curve fitting using GraphPad Prism software. hu2131_Q1E_M99G binds to human CD200R expressing cells with an EC50 of 0.53 nM. Antibody I-4P binds to human CD200R expressing cells with an EC50 of 3.4 nM (FIG. 7).

### Example 11: Binding to Cell Surface Expressed CD200RLa Constructs

In humans and cynomolgus monkeys the inhibitory receptor CD200R has a closely related homolog with activating activity, referred to as CD200RLa. Binding to this receptor would present a safety concern both for humans and also for toxicology studies in cynomolgus monkeys.

Cross-reactivity with the cyno receptor CD200R, but not with the activating homologs, is a desirable property for a CD200R antibody as it enables the use of cynomolgus macaque as a toxicology species.

The binding of CD200R antibodies to human and cyno CD200R and CD200RLa constructs was assessed by flow cytometry of transiently transfected cells. CD200RLa has a charged transmembrane (TM) that is not expected to express well in the absence of appropriate binding partners. Therefore, in addition to the full length CD200RLa constructs, chimeric constructs were used consisting of the CD200RLa extracellular domain fused to mouse CD80 transmembrane and intracellular tail to enable cell surface expression.

HEK293T cells were plated out at 1 million cells per well in a 6 well plate the day before transfection. The following day cells were transfected with CD200R constructs using 2.5 μg plasmid DNA (a pcDNA3.4 vector containing the CD200R construct sequence) and 10 μl Lipofectamine 2000 (mixed in 200 μl Optimem for 5 minutes then added dropwise to cells). 48 hours after transfection, cells were collected by vigorous pipetting in cold PBS and 50,000 cells pelleted per well in 96 well U bottom plates.

Cells were stained with primary antibody diluted in FACS buffer (PBS, 1% FCS, 0.05% sodium azide) to 10 ug/ml for 30 at room temperature. Antibodies tested included the parental murine clone 21.3.1, a chimera with murine 21.3.1 variable domains and human IgG1 P238D Fc, the humanised clones hu2131_v20, hu2131_v20_D56S and hu2131_Q1E_M99G.

Cells were then washed and stained for 30 minutes with a secondary staining mix containing a live/dead marker (Near IR LD, Biolegend cat #423106) and APC anti-hIgG (clone QA19A42, Biolegend cat #366906) or AF647 anti-mIgG1 (clone RMG1-1, cat #406618). As a control, cells were separately stained with the commercial anti-CD200R clone OX108 directly labelled with AF647 (Biolegend cat #329308). Finally, cells were washed and resuspended in FACS buffer for acquisition on a FACS Celesta flow cytometer.

OX108 bound to human CD200R, cyno CD200R and cyno CD200RLa, confirming expression of these constructs. The anti-human CD200RLa clone 6D6C1 (SinoBiological cat #11620-MM09) bound to human CD200RLa, confirming expression of these constructs. The CD200RLa constructs containing the mouse CD80 transmembrane and tail expressed to a higher level than the full length constructs as expected. Clone 2131 in all formats bound to human and cyno CD200R but showed no binding to the activating homolog CD200RLa constructs.

### Example 12: Triggering of DOK2 Recruitment to CD200R Receptor

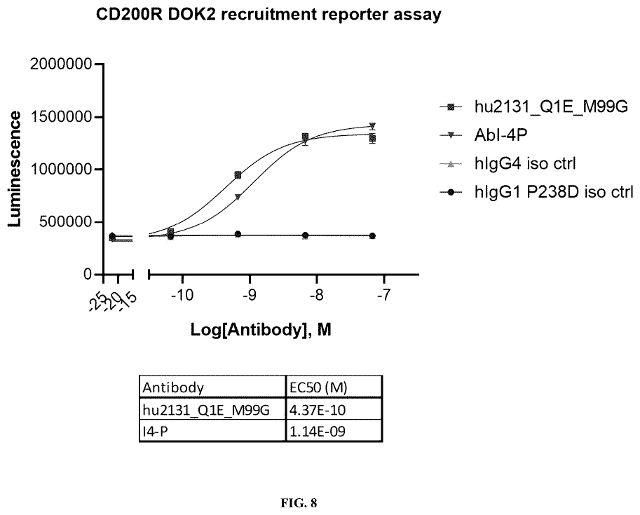

The potency of antibody hu2131_Q1E_M99G and antibody I-4P in triggering Dok2 recruitment to human CD200R was assessed using a PathHunter Jurkat CD200R signalling cell line (Eurofins DiscoverX). This platform employs enzyme fragment complementation to monitor the interaction between CD200R and Dok2, with a chemiluminescent signal generated upon formation of a complete, catalytically active enzyme. PathHunter Jurkat CD200R cells were seeded in a 96-U-bottom plate (2×10⁴ cells/well) and incubated for 24 hours at 37° C. with various concentrations of CD200R antibody or relevant isotype control. In parallel, HEK293 cells expressing FcγRIIb were seeded in a 96-F-bottom plate (2×10⁴ cells/well). The next day, supernatants were removed from the HEK293s and replaced with the PathHunter Jurkat CD200R cell suspensions, which had been pre-incubated with CD200R antibody. After 4 hours' co-culture at 37° C., substrate was added according to manufacturers' instructions (Eurofins DiscoverX) and chemiluminescence measured on a CLARIOstar plate reader. As shown in FIG. 8, antibodies hu2131_Q1E_M99G and I-4P triggered Dok2 recruitment to CD200R, with EC5o values of 0.44 nM and 1.1 nM, respectively.

### Example 13: Specificity Profiling of HuVAR20-2131-D65S

The specificity of antibody hu2131_v20_D56S for binding CD200R was tested in a membrane proteome array provided by Integral molecular. Tested at 20 ug/ml against a cell-array of 6000 human membrane proteins (covering 94% of the human membrane proteome) hu2131_v20_D56S showed binding only to CD200R, with no off-target hits.

### Example 14: Inhibition of the Oxazolone Induced Hypersensitivity Response in Humanized Mice

C57BL/6 mice humanized at the CD200R gene locus will be used to assess the impact of anti-human CD200R agonist antibodies such as 21.3.1 on the hypersensitivity response in a skin challenge model relevant to atopic dermatitis. On Day 0 mice are sensitized with oxazolone by application of 100ul of 2% oxazolone in acetone to their shaved abdomens. Six days after sensitization mice are treated with CD200R antibody or isotype control administered at 0.3, 1, 3, 10 or 30 mg/kg intraperitoneally. Four hours after antibody administration mice are challenged with 10ul of 2% oxazolone in acetone, to the inner surface of one ear. 24 hours after challenge the hypersensitivity reaction is assessed by measuring the difference in ear thickness, or biopsy weight, between the challenged and unchallenged ear. Furthermore, ear tissue is processed to assess inflammatory cytokine levels by ELISA or at the transcript level by qPCR.

CD200R agonist antibodies such as 21.3.1 can lead to a significant inhibition in ear swelling compared to isotype control treated mice.

In a similar study design, mice may be rechallenged with oxazolone to the ear at 14 days, 28 days or 56 days after the first challenge. It is expected that CD200R agonist antibodies such as 21.3.1 and variants thereof can have a durable effect leading to inhibition of ear swelling on rechallenge following the single initial treatment dose.

### Example 15: Inhibition of the Delayed Type Hypersensitivity Response in Humanized Mice

This example provides an experimental illustration of the in vivo inhibitory effect on the delayed type hypersensitivity of some CD200R antibodies according to some embodiments of the present disclosure.

C57BL/6 mice humanized at the CD200R gene locus will be used to assess the impact of anti-human CD200R agonist antibodies such as 21.3.1 on the delayed type hypersensitivity response in a skin challenge model relevant to autoimmune diseases. On Day 0 mice are immunized with keyhole limpet hemocyanin (KLH) in Complete Freund Adjuvant (CFA). The emulsion is a mixture of KLH (Sigma) in PBS, added to CFA (BD Biosciences), at a ratio of 1:1. The final concentration of KLH is 4 mg/mL. Animals are immunized with 100 μL of immunization emulsion injected subcutaneously in 1-2 sites. The unchallenged control group receive just PBS.

On day 0 mice are treated, 1 hour prior to the immunization, with either mIgG1 Isotype control (clone Mopc21) or anti-CD200R antibody intraperitoneally, at a single dose of 10 mg/kg. The unchallenged control group receive PBS. Animals in the positive treatment control group are treated with a CTLA-Ig fusion protein (Biolegend, cat #591908) administered at a dose of 10 mg/kg IP 1 hour prior to the immunization.

Five days after immunization, mice are challenged in the pinna of the left ear (under anesthetic) with 20 μL of 4 mg/mL antigen solution. The unchallenged control group receive 20 μL of PBS in the pinna of the left ear. One day after ear challenge, ear thickness is measured using digital calipers. After measuring ear thickness, animals are humanely sacrificed and, postmortem, an 8 mm diameter circle is cut using a biopsy punch from the left and right ear of each animal from all groups. Ears are weighed on a precise analytical balance. Ear oedema is assessed as the difference between left (challenged) and right (control) ear weight.

CD200R agonist antibodies such as 21.3.1 can lead to a significant inhibition in ear swelling compared to isotype control treated mice.

In a similar study design, to assess the impact of CD200R agonist antibodies such as 21.3.1 on the challenge phase of the response, mice are sensitized as above with KLH on day 0 and treated IP with anti-CD200R antibody or isotype control on day 6, 4 hours prior to ear challenge with KLH.

It is expected that CD200R agonist antibodies such as 21.3.1 will lead to a significant inhibition in ear swelling compared to isotype control treated mice. In an extension to the same study, mice may be rechallenged with KLH to the ear at 28 days or 56 days after the first challenge. It is expected that CD200R agonist antibodies such as 21.3.1 and variants thereof can have a durable effect leading to inhibition of ear swelling on rechallenge following the single initial treatment dose.

### Example 16: Suppression of Human Immune Cell Activation in a Tetanus Toxoid Activation Assay

This example provides an experimental illustration of the in vitro inhibitory effect on the activation of immune cells of some CD200R antibodies according to some embodiments of the present disclosure.

Total human peripheral blood mononuclear cells (PBMCs) from healthy donors (400,000 cells per well of a 96 U-bottom plate) will be stimulated with Tetanus Toxoid (0.5 μg/mL) in the presence of 1pg/ml of anti-CD200R agonist antibody according to an embodiment disclosed herein, 1pg/ml of Antibody I-4P described in Example 5, or 1 pg/ml of human IgGI isotype control. IFNγ release will be assessed by ELISA of supernatant after 96 hours incubation at 37° C., 5% CO2. Six donors will be assessed, and data will be collated by normalizing in each donor to the IFNg level in cells activated with Tetanus Toxoid in the absence of test antibody.

It is expected that average across the donors tested, tetanus toxoid (TT) will induce an approximately 2-fold increase in IFNg production compared to PBMCs culture without TT. It is expected that the anti-CD200R agonist antibody, such as antibody 21.3.1 and variants thereof, can significantly reduce IFNg production compared to the isotype control and Antibody I-4P.

### Example 17: Suppression of Primary T Cell Activation in an Anti-CD3/28 Activation Assay

This example provides an experimental illustration of the in vitro inhibitory effect on the activation of immune cells of some CD200R antibodies according to some embodiments of the present disclosure.

Total human peripheral blood mononuclear cells (PBMCs) from healthy donors (100,000 cells per well of a 96 U-bottom plate) will be stimulated with soluble anti-CD3 and anti-CD28 antibodies (0.5 ng/mL final concentration of each) in the presence of 1pg/ml anti-CD200R agonist antibody or isotype control. CD25 expression on CD4 T cells will be assessed by flow cytometry and cytokine levels in supernatant will be assessed by cytometric bead array, as markers of T cell activation after 72 hours incubation at 37° C., 5% CO2.

Following procedures essentially as described above, hu2131v20 significantly inhibited T cell CD25 expression and inflammatory cytokine production (including IL4, ILS, IL13, IFNg, TNFa, IL17 and IL22) compared to isotype control antibody, or compared to activation in the absence of antibody.

### Example 18: Inhibition of FcεRI-Induced Basophil Activation

This example includes experiments that demonstrate the inhibition of basophil activation (e.g., induced by FcεRI) by exemplary CD200R agonistic antibody according to some embodiments of the present disclosure.

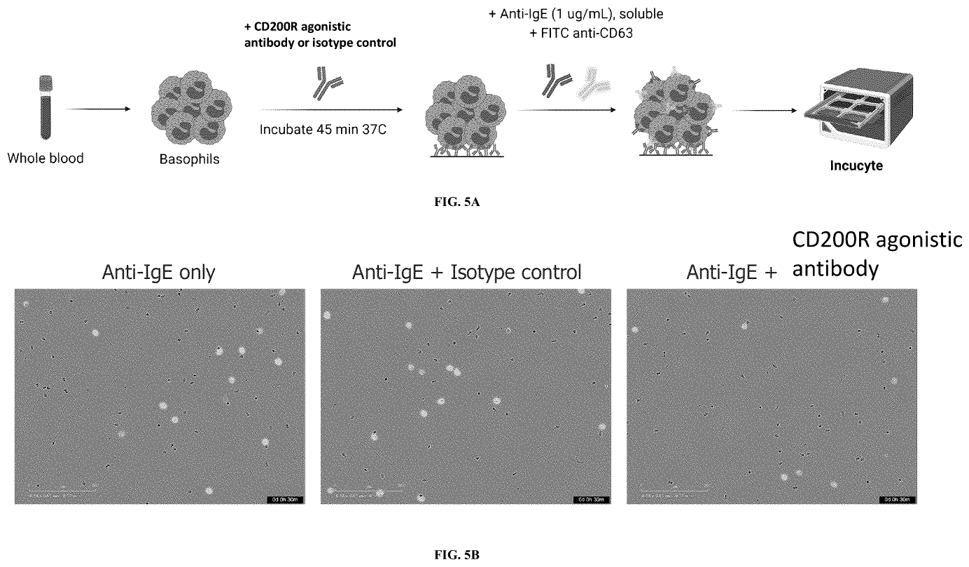

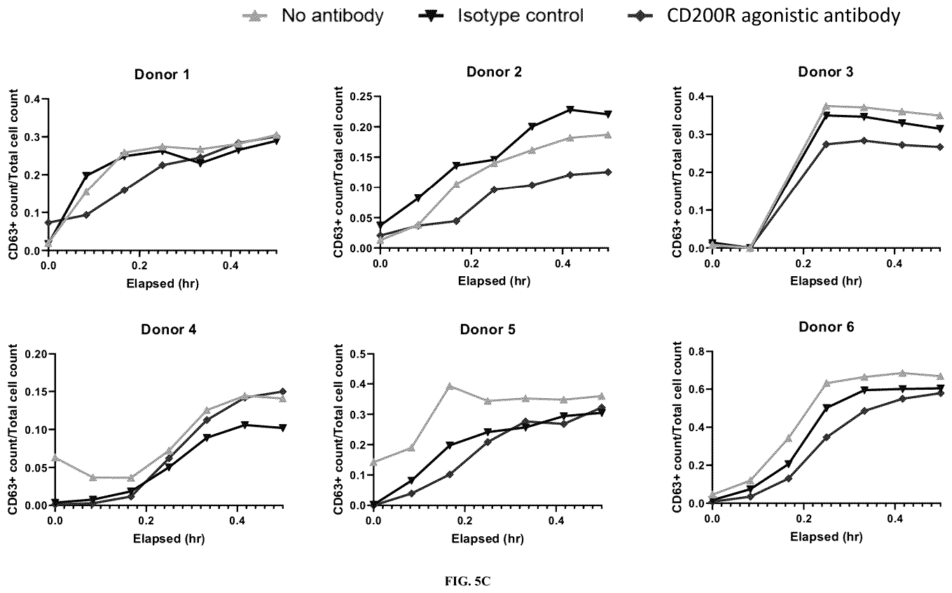

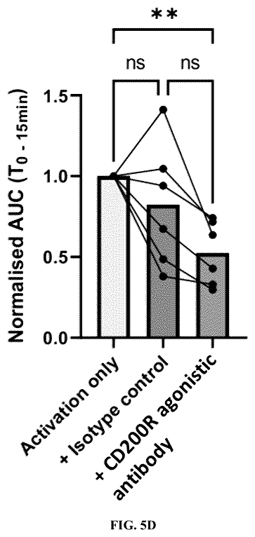

In these experiments, primary human basophils were treated with hu2131v20 or an isotype control of the antibody, then stimulated with anti-IgE antibodies. Activation was monitored by Incucyte imaging, staining with FITC-labelled anti-CD63 antibodies (FIG. 5A). Agonism of CD200R caused a significant decrease in basophil activation, in particular a delay in degranulation (FIGS. 5B-5D).

Briefly, basophils were isolated from the peripheral blood of healthy donors (n=6) and pre-treated with plate-bound CD200R agonistic antibody or isotype control for 45 minutes at 37° C. Cells were then stimulated with soluble anti-IgE antibody in the presence of FITC-conjugated anti-CD63 antibody, and monitored with an Incucyte, imaging every 5 minutes. As shown in FIG. 5B, treatment with the CD200R agonistic antibody significantly reduced the number of light grey objects in the Incucyte images, while treatment with the isotype control did not significantly change the number of light grey objects. The reduction in CD63+positive basophils by CD200R agonistic antibody is also summarized in FIGS. 5C-5D. Incucyte analysis software was utilized to quantify basophil activation, which was expressed as the count of CD63+cells (Light grey objects) divided by the total cell count (Phase objects). Results for individual donors are plotted in FIG. 5C. Area under the curve (AUC) calculations for the first 15 minutes were performed using GraphPad Prism software and normalized to the “activation only” condition for each donor (D). Statistical analysis was performed by one-way ANOVA with Tukey's multiple comparisons test (**, P<0.01).

### Example 19: Gene Expression Regulated by CD200R Agonism

This example demonstrates that CD200R agonism induced by an exemplary CD200R agonistic antibody according to some embodiments of the present disclosure differentially regulates expression of certain genes that are associated with chronic inflammatory conditions.

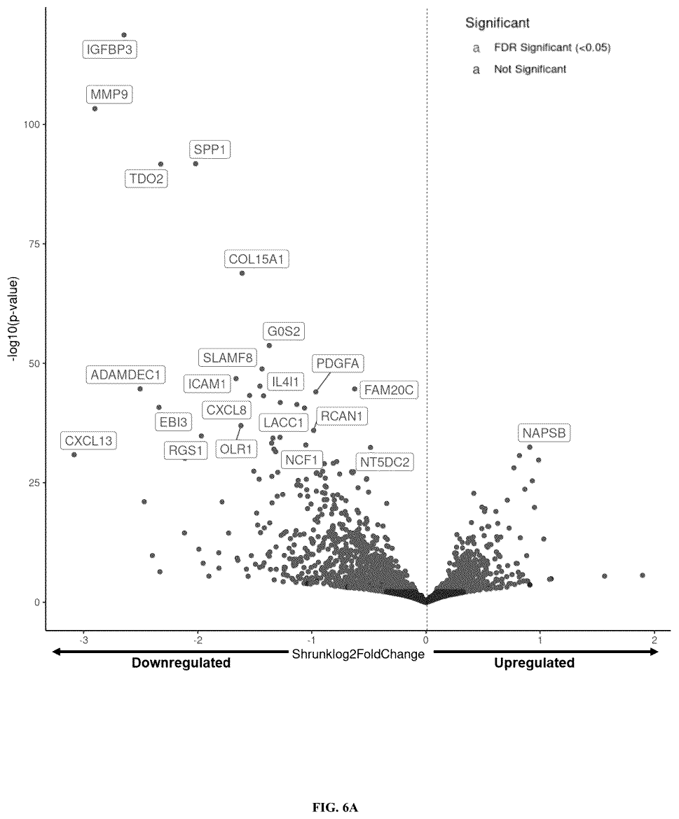

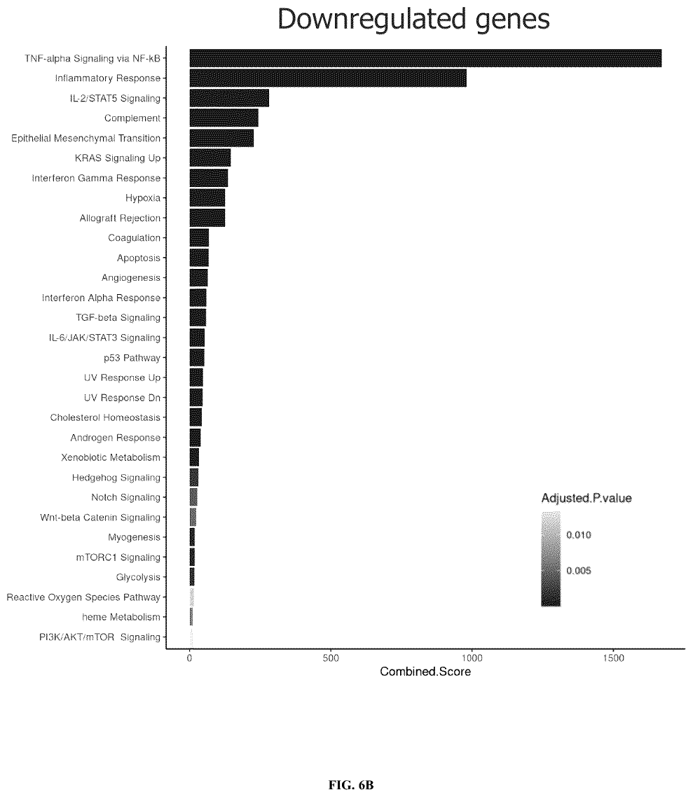

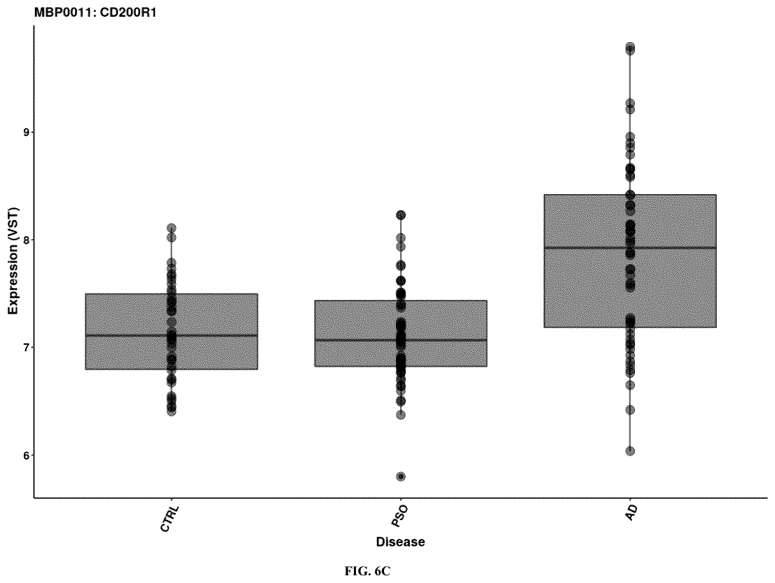

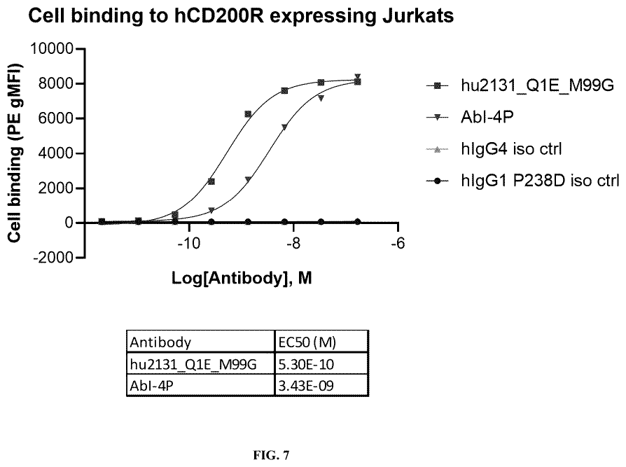

In this example, THP-1 cells expressing CD200R were stimulated with plate-bound human IgGI isotype control in the presence or absence of 1 ug/ml exemplary CD200R agonistic antibody or isotype control. Twenty hours later, cells were harvested for RNAseq analysis. FIG. 6A shows a volcano plot of the differentially expressed genes between THP-1 cells stimulated in the presence of hu2131v20 vs Isotype control. FIG. 6B summarizes gene set enrichment analysis, using MSigDB, of the differentially expressed genes. Gene set enrichment analysis revealed that CD200R agonism downregulated key inflammatory pathways associated with chronic inflammatory conditions. FIG. 6C summarizes CD200R1 expression from RNAseq data of skin biopsies from atopic dermatitis (AD, n=27) and psoriasis (PSO, n=28) patients, as well as healthy controls (CTRL, n=38). As shown in the figure, CD200R1 is upregulated in skin biopsies from atopic dermatitis patients, highlighting the potential benefits of CD200R agonism in this disease setting.

CERTAIN SEQUENCES

SEQ ID NO: 61 (IgG1 P238D-Kabat numbering):
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGDSVF
LFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVF
SCSVMHEALHNHYTQKSLSLSPGK
SEQ ID NO: 62 (KappaLC-homo sapiens):
RTVAAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
SEQ ID NO: 63 (IgG-murine):
AKTTPPSVYPLAPGSAAQTNSMVTLGCLVKGYFPEPVTVTWNSGSLSSGVHTFPAVLQSDL
YTLSSSVTVPSSTWPSETVTCNVAHPASSTKVDKKIVPRDCGCKPCICTVPEVSSVFIFPPKPK
DVLTITLTPKVTCVVVDISKDDPEVQFSWFVDDVEVHTAQTQPREEQFNSTFRSVSELPIMH
QDWLNGKEFKCRVNSAAFPAPIEKTISKTKGRPKAPQVYTIPPPKEQMAKDKVSLTCMITDF
FPEDITVEWQWNGQPAENYKNTQPIMDTDGSYFVYSKLNVQKSNWEAGNTFTCSVLHEGL
HNHHTEKSLSHSPGK
SEQ ID NO: 64 (KappaLC-murine):
RADAAPTVSIFPPSSEQLTSGGASVVCFLNNFYPKDINVKWKIDGSERQNGVLNSWTDQDS
KDSTYSMSSTLTLTKDEYERHNSYTCEATHKTSTSPIVKSFNRNEC
SEQ ID NO: 67 (CDRL2):
GASNLES
SEQ ID NO: 68 (CDRL2):
GASNLET
SEQ ID NO: 69 (CDRH3):
XTGTS, wherein X = M or G
SEQ ID NO: 87 (CDRL2):
XXXNLEX, wherein X at position 1 is G or L, X at position 2 is A or G,
X at position 3 is S or V, and X at position 7 is D, S, or T
SEQ ID NO: 88 (CDRL2):
XAXNLEX, wherein X at position 1 is G or L, X at position 3 is S or V,
X at position 7 is D, S, or T
SEQ ID NO: 89 (CDRL2):
XASNLEX, wherein X at position 1 is G or L and X at position 7 is D,
S, or T
SEQ ID NO: 90 (CDRL2):
GXSNLEX, wherein X at position 2 is A or G and X at position 7 is D,
S, or T
SEQ ID NO: 91 (CDRL3):
LQFTYIPXT, wherein X at position 8 is W or F
SEQ ID NO: 92 (CDRH1):
SYXMH, wherein X at position 3 is W or F
SEQ ID NO: 75 (IgG1-homo sapiens)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTVDKSRWQQGNVFS
CSVMHEALHNHYTQKSLSLSPGK
SEQ ID NO: 76 (IgG4 S228P-homo sapiens)
ASTKGPSVFPLAPCSRSTSESTAALGCLVKDYFPEPVTVSWNSGALTSGVHTFPAVLQSSGL
YSLSSVVTVPSSSLGTKTYTCNVDHKPSNTKVDKRVESKYGPPCPPCPAPEFLGGPSVFLFPP
KPKDTLMISRTPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKSRWQEGNVFSCSV
MHEALHNHYTQKSLSLSLGK
SEQ ID NO: 77 (IgG1 modified)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGXSVF
LFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVF
SCSVMHEALHNHYTQKSLSLSPXX, where X at position 121 is D or P, X at
position 329 is G or absent, and X at position 330 is K or absent
SEQ ID NO: 78 (IgG4 modified)
ASTKGPSVFPLAPCSRSTSESTAALGCLVKDYFPEPVTVSWNSGALTSGVHTFPAVLQSSGL
YSLSSVVTVPSSSLGTKTYTCNVDHKPSNTKVDKRVESKYGPPCPXCPAPEFLGGPSVFLFPP
KPKDTLMISRTPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKSRWQEGNVFSCSV
MHEALHNHYTQKSLSLSLXX, where X at position 108 is S or P, X at position
326 is G or absent, and X at position 327 is K or absent
SEQ ID NO: 82 (hu2131_Q1E_M99G human IgG1 HC full length)
EVQLVQSGAEVKKPGASVKVSCKASGHTFTSYWMHWVRQAPGQGLEWMGNIYPGSGSIN
YDEKFQGRVTMTADTSTSTVYMELSSLRSEDTAVYYCLTGTGTSWGRGTLVTVSSASTKGP
SVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHE
ALHNHYTQKSLSLSPGK
SEQ ID NO: 83 (hu2131_Q1E_M99G human IgG4 S228P HC full length)
EVQLVQSGAEVKKPGASVKVSCKASGHTFTSYWMHWVRQAPGQGLEWMGNIYPGSGSIN
YDEKFQGRVTMTADTSTSTVYMELSSLRSEDTAVYYCLTGTGTSWGRGTLVTVSSASTKGP
SVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYRVVSVLTVLHQ
DWLNGKEYKCKVSNKGLPSSIEKTISKAKGQPREPQVYTLPPSQEEMTKNQVSLTCLVKGF
YPSDIAVEWESNGQPENNYKTTPPVLDSDGSFFLYSRLTVDKSRWQEGNVFSCSVMHEALH
NHYTQKSLSLSLGK
SEQ ID NO: 84 (hu2131_Q1E human IgG1 HC full length)
EVQLVQSGAEVKKPGASVKVSCKASGHTFTSYWMHWVRQAPGQGLEWMGNIYPGSGSIN
YDEKFQGRVTMTADTSTSTVYMELSSLRSEDTAVYYCLTMTGTSWGRGTLVTVSSASTKG
PSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNVFSCSVM
HEALHNHYTQKSLSLSPGK
SEQ ID NO: 85 (hu2131_Q1E human IgG4 S228P HC full length)
EVQLVQSGAEVKKPGASVKVSCKASGHTFTSYWMHWVRQAPGQGLEWMGNIYPGSGSIN
YDEKFQGRVTMTADTSTSTVYMELSSLRSEDTAVYYCLTMTGTSWGRGTLVTVSSASTKG
PSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYRVVSVLTVLHQ
DWLNGKEYKCKVSNKGLPSSIEKTISKAKGQPREPQVYTLPPSQEEMTKNQVSLTCLVKGF
YPSDIAVEWESNGQPENNYKTTPPVLDSDGSFFLYSRLTVDKSRWQEGNVFSCSVMHEALH
NHYTQKSLSLSLGK
SEQ ID NO: 93 (VH):
XVQLVQSGAEVKKPGASVKVSCKASGHTFTSYXMHWVRQAPGQGLEWMGNIYPGSGSIN
YDEKFQGRVTMTADTSTSTVYMELSSLRSEDTAVYYCLTXTGTSWGRGTLVTVSS, where X
at position 1 is Q or E, X at position 33 is W or F, and X at position
99 is M or G
SEQ ID NO: 94 (VL):
DIQMTQSPSSLSVSVGDRVTITCQASQGTPINLHWFQQKPGKAPKLLISXXXNLEXGVPSRFS
GSGSGTEFTFTISSLQPEDAATYYCLQFTYIPXTFGQGTKLEIK, where X at position 50
is G or L, X at position 51 is A or G, X at position 52 is S or V, X
at position 56 is D, S, or T, and X at position 96 is W or F
SEQ ID NO: 95 (HC full length-IgG1)
XVQLVQSGAEVKKPGASVKVSCKASGHTFTSYXMHWVRQAPGQGLEWMGNIYPGSGSIN
YDEKFQGRVTMTADTSTSTVYMELSSLRSEDTAVYYCLTXTGTSWGRGTLVTVSSASTKGP
SVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGXSVFLFPPKP
KDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNVFSCSVM
HEALHNHYTQKSLSLSPXX, where X at position 1 is D or E, X at position 33
is W or F, X at position 99 is M or G, X at position 235 is D or P, X
at position 443 is G or absent, and X at position 444 is K or absent
SEQ ID NO: 96 (HC full length-IgG4)
XVQLVQSGAEVKKPGASVKVSCKASGHTFTSYXMHWVRQAPGQGLEWMGNIYPGSGSIN
YDEKFQGRVTMTADTSTSTVYMELSSLRSEDTAVYYCLTXTGTSWGRGTLVTVSSASTKGP
SVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPXCPAPEFLGGPSVFLFPPKPKDT
LMISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGNVFSCSVMHEAL
HNHYTQKSLSLSLXX, where X at position 1 is D or E, X at position 33 is
W or F, X at position 99 is M or G, X at position 222 is S or P, X at
position 440 is G or absent, and X at position 441 is K or absent
SEQ ID NO: 97 (HC full length-IgG1 murine)
XVQLVQSGAEVKKPGASVKVSCKASGHTFTSYXMHWVRQAPGQGLEWMGNIYPGSGSIN
YDEKFQGRVTMTADTSTSTVYMELSSLRSEDTAVYYCLTXTGTSWGRGTLVTVSSAKTTPP
SVYPLAPGSAAQTNSMVTLGCLVKGYFPEPVTVTWNSGSLSSGVHTFPAVLQSDLYTLSSS
VTVPSSTWPSETVTCNVAHPASSTKVDKKIVPRDCGCKPCICTVPEVSSVFIFPPKPKDVLTIT
LTPKVTCVVVDISKDDPEVQFSWFVDDVEVHTAQTQPREEQFNSTFRSVSELPIMHQDWLN
GKEFKCRVNSAAFPAPIEKTISKTKGRPKAPQVYTIPPPKEQMAKDKVSLTCMITDFFPEDIT
VEWQWNGQPAENYKNTQPIMDTDGSYFVYSKLNVQKSNWEAGNTFTCSVLHEGLHNHHT
EKSLSHSPXX, where X at position 1 is D or E, X at position 33 is W or
F, X at position 99 is M or G, X at position 437 is G or absent, and X
at position 438 is K or absent
SEQ ID NO: 98 (LC full length-K):
DIQMTQSPSSLSVSVGDRVTITCQASQGTPINLHWFQQKPGKAPKLLISXXXNLEXGVPSRFS
GSGSGTEFTFTISSLQPEDAATYYCLQFTYIPXTFGQGTKLEIKRTVAAPSVFIFPPSDEQLKS
GTASVVCLLNNFYPREAKVQWKVDNALQSGNSQESVTEQDSKDSTYSLSSTLTLSKADYE
KHKVYACEVTHQGLSSPVTKSFNRGEC, where X at position 50 is G or L, X at
position 51 is A or G, X at position 52 is S or V, X at position 56 is
D, S, or T, and X at position 96 is W or F

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1. (canceled)

2. An antibody or an antigen-binding fragment thereof that specifically binds CD200R, comprising a heavy chain and a light chain, wherein the heavy chain comprises a heavy chain variable region (HCVR) that comprises heavy chain complementarity determining region 1 (CDRH1), CDRH2, and CDRH3, and wherein CDRH1, CDRH2, and CDRH3 comprise the sequence as set forth in
a) SEQ ID NOs: 3, 41, and 70, respectively, each with 0 to 3 amino acid modifications, such as 0, 1, 2, or 3 modifications;
b) SEQ ID NOs: 3, 41, and 69, respectively, each with 0 to 3 amino acid modifications, such as 0, 1, 2, or 3 modifications;
c) SEQ ID NOs: 3, 41, and 5, respectively, each with 0 to 3 amino acid modifications, such as 0, 1, 2, or 3 modifications;
d) SEQ ID NOs: 3, 4, and 5, respectively, each with 0 to 3 amino acid modifications, such as 0, 1, 2, or 3 modifications;
e) SEQ ID NOs: 11, 12, and 13, respectively, each with 0 to 3 amino acid modifications, such as 0, 1, 2, or 3 modifications;
f) SEQ ID NOs: 19, 20, and 21, respectively, each with 0 to 3 amino acid modifications, such as 0, 1, 2, or 3 modifications;
g) SEQ ID NOs: 27, 28, and 29, respectively, each with 0 to 3 amino acid modifications, such as 0, 1, 2, or 3 modifications;
h) SEQ ID NOs: 35, 36, and 37, respectively, each with 0 to 3 amino acid modifications, such as 0, 1, 2, or 3 modifications; or
i) SEQ ID NOs: 92, 41, and 69; and
wherein the light chain comprises a light chain variable region (LCVR) that comprises light chain complementarity determining region 1 (CDRL1), CDRL2, and CDRL3, and wherein CDRL1, CDRL2, and CDRL3 comprise the sequence as set forth in:
j) SEQ ID NOs: 6, 67, and 8, respectively, each with 0 to 3 amino acid modifications, such as 0, 1, 2, or 3 modifications:
k) SEQ ID NOs: 6, 7, and 8, respectively, each with 0 to 3 amino acid modifications, such as 0, 1, 2, or 3 modifications:
1) SEQ ID NOs: 14, 15, and 16, respectively, each with 0 to 3 amino acid modifications, such as 0, 1, 2, or 3 modifications:
m) SEQ ID NOs: 22, 23, and 24, respectively, each with 0 to 3 amino acid modifications, such as 0, 1, 2, or 3 modifications:
n) SEQ ID NOs: 30, 31, and 32, respectively, each with 0 to 3 amino acid modifications, such as 0, 1, 2, or 3 modifications:
o) SEQ ID NOs: 38, 39, and 40, respectively, each with 0 to 3 amino acid modifications, such as 0, 1, 2, or 3 modifications: or
p) SEQ ID NOs: 6, 68, and 8, respectively, each with 0 to 3 amino acid modifications, such as 0, 1, 2, or 3 modifications.

3.-9. (canceled)

10. The antibody or antigen-binding fragment thereof of claim 2, wherein:
a) the HCVR comprises an amino acid sequence having at least 90%, 95%, 96%, 97%, 98%, 99%, or 100% sequence identity to the amino acid sequence as set forth in SEQ ID NO: 72, and the LCVR comprises an amino acid sequence having at least 90%, 95%, 96%, 97%, 98%, 99%, or 100% sequence identity to the amino acid sequence as set forth in SEQ ID NO: 65; and/or the HCVR comprises an amino acid sequence as set forth in SEQ ID NO: 72, with from 0 to 10 amino acid modifications, such as 1, 2, 3, 4, 5, 6, 7, 8, 9 or 10 amino acid modifications, and the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications:
b) the HCVR comprises an amino acid sequence having at least 90%, 95%, 96%, 97%, 98%, 99%, or 100% sequence identity to the amino acid sequence as set forth in SEQ ID NO: 71, and the LCVR comprises an amino acid sequence having at least 90%, 95%, 96%, 97%, 98%, 99%, or 100% sequence identity to the amino acid sequence as set forth in SEQ ID NO: 65; and/or the HCVR comprises an amino acid sequence as set forth in SEQ ID NO: 71, with from 0 to 3 amino acid modifications, and the LCVR comprises an amino acid sequence as set forth in SEQ ID NO: 65, with from 0 to 3 amino acid modifications:
c) the HCVR comprises an amino acid sequence having at least 90%, 95%, 96%, 97%, 98%, 99%, or 100% sequence identity to the amino acid sequence as set forth in SEQ ID NO: 1, and the LCVR comprises an amino acid sequence having at least 90%, 95%, 96%, 97%, 98%, 99%, or 100% sequence identity to the amino acid sequence as set forth in SEQ ID NO: 2; and/or the HCVR comprises an amino acid sequence as set forth in SEQ ID NO: 1, with from 0 to 10 amino acid modifications, such as 1, 2, 3, 4, 5, 6, 7, 8, 9 or 10 amino acid modifications, and the LCVR comprises an amino acid sequence as set forth in SEQ ID NO: 2, with from 0 to 10 amino acid modifications, such as 1, 2, 3, 4, 5, 6, 7, 8, 9 or 10 amino acid modifications:
d) the HCVR comprises an amino acid sequence having at least 90%, 95%, 96%, 97%, 98%, 99%, or 100% sequence identity to the amino acid sequence as set forth in SEQ ID NO: 9, and the LCVR comprises an amino acid sequence having at least 90%, 95%, 96%, 97%, 98%, 99%, or 100% sequence identity to the amino acid sequence as set forth in SEQ ID NO: 10: and/or the HCVR comprises an amino acid sequence as set forth in SEQ ID NO: 9, with from 0 to 10 amino acid modifications, such as 1, 2, 3, 4, 5, 6, 7, 8, 9 or 10 amino acid modifications, and the LCVR comprises an amino acid sequence as set forth in SEQ ID NO: 10, with from 0 to 10 amino acid modifications, such as 1, 2, 3, 4, 5, 6, 7, 8, 9 or 10 amino acid modifications;
e) the HCVR comprises an amino acid sequence having at least 90%, 95%, 96%, 97%, 98%, 99%, or 100% sequence identity to the amino acid sequence as set forth in SEQ ID NO: 17, and the LCVR comprises an amino acid sequence having at least 90%, 95%, 96%, 97%, 98%, 99%, or 100% sequence identity to the amino acid sequence as set forth in SEQ ID NO: 18; and/or e) the HCVR comprises an amino acid sequence as set forth in SEQ ID NO: 17, with from 0 to 10 amino acid modifications, such as 1, 2, 3, 4, 5, 6, 7, 8, 9 or 10 amino acid modifications, and the LCVR comprises an amino acid sequence as set forth in SEQ ID NO: 18, with from 0 to 10 amino acid modifications, such as 1, 2, 3, 4, 5. 6, 7, 8, 9 or 10 amino acid modifications:
f) the HCVR comprises an amino acid sequence having at least 90%, 95%, 96%, 97%, 98%, 99%, or 100% sequence identity to the amino acid sequence as set forth in SEQ ID NO: 25, and the LCVR comprises an amino acid sequence having at least 90%, 95%, 96%, 97%, 98%, 99%, or 100% sequence identity to the amino acid sequence as set forth in SEQ ID NO: 26; and/or the HCVR comprises an amino acid sequence as set forth in SEQ ID NO: 25, with from 0 to 10 amino acid modifications, such as 1, 2, 3, 4, 5, 6, 7, 8, 9 or 10 amino acid modifications, and the LCVR comprises an amino acid sequence as set forth in SEQ ID NO: 26, with from 0 to 10 amino acid modifications, such as 1, 2, 3, 4, 5. 6, 7, 8, 9 or 10 amino acid modifications:
g) the HCVR comprises an amino acid sequence having at least 90%, 95%, 96%, 97%, 98%, 99%, or 100% sequence identity to the amino acid sequence as set forth in SEQ ID NO: 33, and the LCVR comprises an amino acid sequence having at least 90%, 95%, 96%, 97%, 98%, 99%, or 100% sequence identity to the amino acid sequence as set forth in SEQ ID NO: 34; and/or the HCVR comprises an amino acid sequence as set forth in SEQ ID NO: 33, with from 0 to 3 amino acid modifications, and the LCVR comprises an amino acid sequence as set forth in SEQ ID NO: 34, with from 0 to 3 amino acid modifications:
h) the HCVR comprises an amino acid sequence having at least 90%, 95%, 96%, 97%, 98%, 99%, or 100% sequence identity to the amino acid sequence as set forth in any one of SEQ ID NOs: 1, 42, 43, 44, 45, 46, 47, 71, 72, or 93 and the LCVR comprises an amino acid sequence having at least 90%, 95%, 96%, 97%, 98%, 99%, or 100% sequence identity to the amino acid sequence as set forth in SEQ ID NO: 2, 48, 49, 50, 51, 52, 65, 66, or 94; and/or the HCVR comprises an amino acid sequence as set forth in any one of SEQ ID NOs: 1, 42, 43, 44, 45, 46, 47, 71, 72, or 93 with from 0 to 10 amino acid modifications, such as 1, 2, 3, 4, 5, 6, 7, 8, 9 or 10 amino acid modifications, and the LCVR comprises an amino acid sequence as set forth in SEQ ID NOs: 2, 48, 49, 50, 51, 52, 65, 66, or 94, with from 0 to 10 amino acid modifications, such as 1, 2, 3, 4, 5, 6, 7, 8, 9 or 10 amino acid modifications: or
i) the HCVR comprises an amino acid sequence having at least 90%, 95%, 96%, 97%, 98%, 99%, or 100% sequence identity to the amino acid sequence as set forth in any one of SEQ ID NOs: 42, 43, 44, 45, 46, 47, 71, 72, or 93 and the LCVR comprises an amino acid sequence having at least 90%, 95%, 96%, 97%, 98%, 99%, or 100% sequence identity to the amino acid sequence as set forth in SEQ ID NOS: 48, 49, 50, 51, 52, 65, 66, or 94: and/or HCVR comprises an amino acid sequence as set forth in any one of SEQ ID NOs: 42, 43, 44, 45, 46, 47, 71, 72, or 93 with from 0 to 10 amino acid modifications, such as 1, 2, 3, 4, 5, 6, 7, 8, 9 or 10 amino acid modifications, and the LCVR comprises an amino acid sequence as set forth in SEQ ID NOs: 48, 49, 50, 51, 52, 65, 66, or 94 with from 0 to 10 amino acid modifications, such as 1, 2, 3, 4, 5, 6, 7, 8, 9 or 10 amino acid modifications.

11.-21. (canceled)

22. The antibody or an antigen-binding fragment thereof of claim 2 wherein:
(a) the HCVR comprises an amino acid sequence having at least 90%, 95%, 96%, 97%, 98%, 99%, or 100% sequence identity to the amino acid sequence as set forth in SEQ ID NO: 72, and the LCVR comprises an amino acid sequence having at least 90%, 95%, 96%, 97%, 98%, 99%, or 100% sequence identity to the amino acid sequence as set forth in SEQ ID NO: 65, wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 70, respectively; and the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively;
(b) the HCVR comprises an amino acid sequence as set forth in SEQ ID NO: 72, with from 0 to 10 amino acid modifications, such as 1, 2, 3, 4, 5, 6, 7, 8, 9 or 10 amino acid modifications, and the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications, wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 70, respectively; and the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively;
(c) the HCVR comprises an amino acid sequence having at least 90%, 95%, 96%, 97%, 98%, 99%, or 100% sequence identity to the amino acid sequence as set forth in SEQ ID NO: 71, and the LCVR comprises an amino acid sequence having at least 90%, 95%, 96%, 97%, 98%, 99%, or 100% sequence identity to the amino acid sequence as set forth in SEQ ID NO: 65, wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 5, respectively; and the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively;
(d) the HCVR comprises an amino acid sequence as set forth in SEQ ID NO: 71, with from 0 to 10 amino acid modifications, such as 1, 2, 3, 4, 5, 6, 7, 8, 9 or 10 amino acid modifications, and the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications, wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 5, respectively; and the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively;
(e) the HCVR comprises an amino acid sequence having at least 90%, 95%, 96%, 97%, 98%, 99%, or 100% sequence identity to the amino acid sequence as set forth in SEQ ID NO: 93, and the LCVR comprises an amino acid sequence having at least 90%, 95%, 96%, 97%, 98%, 99%, or 100% sequence identity to the amino acid sequence as set forth in SEQ ID NO: 94, wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 92, 41, and 69, respectively; and the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 87, and 91, respectively; or
(f) the HCVR comprises an amino acid sequence as set forth in SEQ ID NO: 93, with from 0 to 10 amino acid modifications, such as 1, 2, 3, 4, 5, 6, 7, 8, 9 or 10 amino acid modifications, and the LCVR comprises an amino acid sequence as set forth in SEQ ID NO: 94, with from 0 to 10 amino acid modifications, such as 1, 2, 3, 4, 5, 6, 7, 8, 9 or 10 amino acid modifications, wherein the HCVR comprises heavy chain complementarity determining region 1 (CDRH1), CDRH2, and CDRH3, and wherein CDRH1, CDRH2, and CDRH3 comprise the sequence as set forth in SEQ ID NOs: 92, 41, and 69, respectively; and the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 87, and 91, respectively.

23.-25. (canceled)

26. The antibody or an antigen-binding fragment thereof of claim 22, wherein:
(a) X at position 1 of SEQ ID NO: 93 is D;
(b) X at position 1 of SEQ ID NO: 93 is E;
(c) X at position 33 of SEQ ID NO: 93 is W;
(d) X at position 33 of SEQ ID NO: 93 is F;
(e) X at position 99 of SEQ ID NO: 93 is M;
(f) X at position 99 of SEQ ID NO: 93 is G;
(g) X at position 50 of SEQ ID NO: 94 is G;
(h) X at position 50 of SEQ ID NO: 94 is L;
(i) X at position 51 of SEQ ID NO: 94 is A;
(j) X at position 51 of SEQ ID NO: 94 is G;
(k) X at position 52 of SEQ ID NO: 94 is S;
(1) X at position 52 of SEQ ID NO: 94 is V;
(m)X at position 56 of SEQ ID NO: 94 is D;
(n) X at position 56 of SEQ ID NO: 94 is S;
(o) X at position 56 of SEQ ID NO: 94 is T;
(p) X at position 96 of SEQ ID NO: 94 is W;
(q) X at position 96 of SEQ ID NO: 94 is F;
(r) any combination of (a) to (q),
(s) X at position 1 of SEQ ID NO: 69 is M:
(t) X at position 1 of SEQ ID NO: 69 is G:
(u) X at position 8 of SEQ ID NO: 91 is W:
(v) X at position 8 of SEQ ID NO: 91 is F:
(w) X at position 3 of SEQ ID NO: 92 is W:
(x) X at position 3 of SEQ ID NO: 92 is F:
(v) X at position 1 of SEQ ID NO: 87 is G:
(z) X at position 1 of SEQ ID NO: 87 is L:
(z1) X at position 2 of SEQ ID NO: 87 is A;
(z2) X at position 2 of SEQ ID NO: 87 is G;
(z3) X at position 3 of SEQ ID NO: 87 is S;
(z4) X at position 3 of SEQ ID NO: 87 is V;
(z5) X at position 7 of SEQ ID NO: 87 is D;
(z6) X at position 7 of SEQ ID NO: 87 is S;
(z7) X at position 7 of SEQ ID NO: 87 is T; or
(z8) any combination of (s) to (z7).

27. (canceled)

28. The antibody or antigen-binding fragment thereof of claim 2, wherein
(a) the heavy chain or light chain further comprise a constant region;
(b) the heavy chain and light chain are connected by a flexible linker to form a single-chain antibody;
(c)
(i) the LCVR is linked to a light chain constant region comprising an amino acid sequence having at least 90%, 95%, 96%, 97%, 98%, 99%, or 100% sequence identity to the amino acid sequence as set forth in SEQ ID NO: 62, or the LCVR is linked to a light chain constant region comprising an amino acid sequence as set forth in SEQ ID NO: 62, with from 0 to 10 amino acid modifications, such as 1, 2, 3, 4, 5, 6, 7, 8, 9 or 10 amino acid modifications: or
(ii) the LCVR is linked to a light chain constant region comprising an amino acid sequence having at least 90%, 95%, 96%, 97%, 98%, 99%, or 100% sequence identity to the amino acid sequence as set forth in SEQ ID NO: 64, or the LCVR is linked to a light chain constant region comprising an amino acid sequence as set forth in SEQ ID NO: 64, with from 0 to 10 amino acid modifications, such as 1, 2, 3, 4, 5, 6, 7, 8, 9 or 10 amino acid modifications:
(d)
(i) the HCVR is linked to a heavy chain constant region comprising an amino acid sequence having at least 90%, 95%, 96%, 97%, 98%, 99%, or 100% sequence identity to the amino acid sequence as set forth in SEQ ID NOS: 61, 63, 75, and 76, optionally, the HCVR is linked to a heavy chain constant region comprising an amino acid sequence having at least 90%, 95%, 96%, 97%, 98%, 99%, or 100% sequence identity to the amino acid sequence as set forth in SEQ ID NOS: 61, 75, and 76;
(ii) the HCVR is linked to a heavy chain constant region comprising an amino acid sequence as set forth in SEQ ID NOS: 61, 63, 75, and 76, with from 0 to 10 amino acid modifications, such as 1, 2, 3, 4, 5, 6, 7, 8, 9 or 10 amino acid modifications, optionally, the HCVR is linked to a heavy chain constant region comprising an amino acid sequence as set forth in SEQ ID NOS: 61, 75, and 76, with from 0 to 10 amino acid modifications, such as 1, 2, 3, 4, 5, 6, 7, 8, 9 or 10 amino acid modifications: or
(e) wherein the antibody or an antigen-binding fragment thereof comprises:
(i) a heavy chain of SEQ ID NO: 82 and [[(b) 1]]a light chain of SEQ ID NO: 86:
(ii) a heavy chain of SEQ ID NO: 83 and a light chain of SEQ ID NO: 86:
(iii) a heavy chain of SEQ ID NO: 84 and a light chain of SEQ ID NO: 86.
(iv) a heavy chain of SEQ ID NO: 85 and a light chain of SEQ ID NO: 86.
(v) a heavy chain of SEQ ID NO: 95 and a light chain of SEQ ID NO: 98.
(vi) a heavy chain of SEQ ID NO: 96 and a light chain of SEQ ID NO: 98; or
(vii) a heavy chain of SEQ ID NO: 97 and a light chain of SEQ ID NO: 98.

29.-42. (canceled)

43. The antibody or antigen-binding fragment thereof of claim 28, wherein:
(1) for the heavy chain of SEQ ID NO: 95:
(a) X at position 1 of SEQ ID NO: 95 is D;
(b) X at position 1 of SEQ ID NO: 95 is E;
(c) X at position 33 of SEQ ID NO: 95 is W;
(d) X at position 33 of SEQ ID NO: 95 is F;
(e) X at position 99 of SEQ ID NO: 95 is M;
(f) X at position 99 of SEQ ID NO: 95 is G;
(g) X at position 235 of SEQ ID NO: 95 is D;
(h) X at position 235 of SEQ ID NO: 95 is P;
(i) X at position 443 of SEQ ID NO: 95 is G;
(j) X at positions 443 and 444 of SEQ ID NO: 95 are absent;
(k) X at position 444 is SEQ ID NO: 95 is K;
(l) X at position 444 is SEQ ID NO: 95 is absent; or
(m) any combination of (a) to (1);
(2) for the heavy chain of SEQ ID NO 96:
(a) X at position 1 of SEQ ID NO: 96 is D:
(b) X at position 1 of SEQ ID NO: 96 is E:
(c) X at position 33 of SEQ ID NO: 96 is W;
(d) X at position 33 of SEQ ID NO: 96 is F;
(e) X at position 99 of SEQ ID NO: 96 is M;
(f) X at position 99 of SEQ ID NO: 96 is G;
(g) X at position 222 of SEQ ID NO: 96 is S;
(h) X at position 222 of SEQ ID NO: 96 is P;
(i) X at position 440 of SEQ ID NO: 96 is G;
(i) X at positions 440 and 441 of SEQ ID NO: 96 are absent:
(k) X at position 441 of SEQ ID NO: 96 is K:
(l) X at position 441 of SEQ ID NO: 96 is absent: or
(m) any combination of (a) to (1)
(3) for the heavy chain of SEQ ID NO: 97:
(a) X at position 1 of SEQ ID NO: 97 is D;
(b) X at position 1 of SEQ ID NO: 97 is E;
(c) X at position 33 of SEQ ID NO: 97 is W;
(d) X at position 33 of SEQ ID NO: 97 is F;
(e) X at position 99 of SEQ ID NO: 97 is M;
(f) X at position 99 of SEQ ID NO: 97 is G;
(g) X at position 437 of SEQ ID NO: 97 is G;
(h) X at position 437 and 438 of SEQ ID NO: 97 are absent:
(i) X at position 438 of SEQ ID NO: 97 is K:
(i) X at position 438 of SEQ ID NO: 97 is absent: or
(k) any combination of (a) to (j);
and/or wherein
for the light chain of SEQ ID NO: 98:
(a) X at position 50 of SEQ ID NO: 98 is G.
(b) X at position 50 of SEQ ID NO: 98 is L;
(c) X at position 51 of SEQ ID NO: 98 is A;
(d) X at position 51 of SEQ ID NO: 98 is G;
(e) X at position 52 of SEQ ID NO: 98 is S;
(f) X at position 52 of SEQ ID NO: 98 V.
(g) X at position 56 of SEQ ID NO: 98 is D;
(h) X at position 56 of SEQ ID NO: 98 is S;
(i) X at position 56 of SEQ ID NO: 98 is T;
(i) X at position 96 of SEQ ID NO: 98 is W;
(k) X at position 96 of SEQ ID NO: 98 is F; or
(l) any combination of (a) to (k).

44.-48. (canceled)

49. The antibody or antigen-binding fragment thereof of claim 2, wherein
(a) the HCVR is linked to a heavy chain constant region, wherein the heavy chain constant region comprises an Fc region that comprises one or more of the following amino acids: alanine (A) at position 234, alanine (A) at position 235, aspartic acid (D) at position 236, aspartic acid (D) at position 237, aspartic acid (D) at position 238, alanine (A) at position 265, glutamic acid (E) at position 267, glycine (G) at position 271, arginine (R) at position 330, alanine (A) at position 332, or alanine (A) at position 297 (numbering according to EU Index), optionally wherein the Fc region comprises an aspartic acid (D) at position 238 (EU Index):
(b) the HCVR is linked to a heavy chain constant region comprising an amino acid sequence having at least 90%, 95%, 96%, 97%, 98%, 99%, or 100% sequence identity to the amino acid sequence as set forth in SEQ ID NO: 77, or the HCVR is linked to a heavy chain constant region comprising an amino acid sequence as set forth in SEQ ID NO: 77, with from 0 to 10 amino acid modifications, such as 1, 2, 3, 4, 5, 6, 7, 8, 9 or 10 amino acid modifications, optionally wherein X at position 121 of SEQ ID NO: 77 is D or P, X at position 329 of SEQ ID NO: 77 is G or absent, or X at position 330 of SEQ ID NO: 77 is K or absent:
(c) the HCVR is linked to a heavy chain constant region comprising an amino acid sequence having at least 90%, 95%, 96%, 97%, 98%, 99%, or 100% sequence identity to the amino acid sequence as set forth in SEQ ID NO: 78, or the heavy chain variable region is linked to a heavy chain constant region comprising an amino acid sequence as set forth in SEQ ID NO: 78, with from 0 to 10 amino acid modifications, such as 1, 2, 3, 4, 5, 6, 7, 8, 9 or 10 amino acid modifications, optionally wherein X at position 108 of SEQ ID NO: 78 is S or P, X at position 326 of SEQ ID NO: 78 is G or absent, or X at position 327 of SEQ ID NO: 78 is K or absent:
(d) the antibody or antigen-binding fragment thereof is an IgG, an IgM, an IgE, an IgA, or an IgD molecule, or is derived from one of these:
(e) the antibody or antigen-binding fragment thereof is an IgG1, IgG2, IgG3, or IgG4 molecule, or is derived from one of these:
(f) the antibody is an IgGI antibody: or
(g) the antigen-binding fragment is selected from the group consisting of: scFv, sc(Fv)2, dsFv, Fab, Fab′, (Fab′)2 and a diabody.

50.-74. (canceled)

75. The antibody or antigen-binding fragment thereof of claim 2, wherein
(a) the antibody is an IgGI antibody, optionally wherein
(1) the IgGI antibody comprises one or more modifications at positions 234, 235, 236, 238, 239, 243, 250, 252, 254, 256, 257, 292, 297, 311, 322, 326, 329, 330, 332, 333, 396, 428, 433, and 434 (EU index);
(2) the IgGI antibody comprises one or more modifications selected from L234 Å, L235 Å, L235V, G236 Å, P238D: S239D, F243L, T250Q, M252Y, S254T, T256E, P2571, R292P, N297D, Q311, K322 Å, K326W, P329 Å, P329G, A330L, 1332E, E333 Å, E333S, P396L, M428L, H433K, and N434F (EU index):
(3) the IgGI antibody comprises one or more modifications selected from:
(i) S239D, A330L, and 1332E (EU index):
(ii) L234A and L235A (EU index):
(iii) T250Q and M428L (EU index);
(iv) M252Y, S254T, T256E, H433K, and N434F (EU index):
(v) E333A (EU index);
(vi) P257I and Q311 (EU index);
(vii) K326W and E333 S (EU index):
(viii) S239D, 1332E, and G236A (EU index):
(ix) K322A (EU index): and
(x) P238D (EU index):
(4) the IgGI antibody comprises a P238D substitution;
(b) the antibody is an IgG2 antibody, optionally wherein the IgG2 antibody is derived from a mouse IgG2 antibody; optionally wherein the mouse IgG2 antibody comprises one or more modifications selected from L235E, E318 Å, K320 Å, and K322A (EU index):
(c) the antibody is an IgG3 antibody; or
(d) the antibody is an IgG4 antibody, optionally wherein
(1) the IgG4 antibody comprises one or more modifications at positions 228, 234, 235, 327, 329, 330 and 331 (EU index):
(2) the IgG4 antibody comprises one or more modifications selected from S228P, L234F, L235E, A327G, P329G, A330S and P331S (EU index): or
(3) the IgG4 antibody comprises a S228P substitution (EU index).

76.-87. (canceled)

88. The antibody or antigen-binding fragment thereof of claim 2, wherein
(a) the antibody or antigen-binding fragment thereof binds a region at or in proximity of C-terminus of the extracellular portion of CD200, the antibody or antigen-binding fragment thereof binds a region at most 50 amino acids, 45 amino acids, 40 amino acids, 35 amino acids, 30 amino acids, 25 amino acids, 20 amino acids, or 15 amino acids from the C-terminus of the extracellular portion of CD200R, the antibody or antigen-binding fragment thereof binds a region about 50 amino acids, 45 amino acids, 40 amino acids, 35 amino acids, 30 amino acids, 25 amino acids, 20 amino acids, 18 amino acids, 16 amino acids, 15 amino acids, 14 amino acids, 13 amino acids, 12 amino acids, 10 amino acids, 8 amino acids, 6 amino acids, or 5 amino acids from the C-terminus of the extracellular portion of CD200R, and/or the antibody or antigen-binding fragment thereof binds a region at most 100 Å, 90 Å, 80 Å, 70 Å, 60 Å, 50 Å, 40 A, 30 Å, 20 Å, or 10 A from the cell membrane when the antibody or antigen-binding fragment thereof binds to a CD200R molecule on the cell membrane:
(b) antibody or antigen-binding fragment thereof binds a region in proximity of N-terminus of CD200R:
(c) the antibody or antigen-binding fragment thereof binds a residue of CD200R selected from T213, E230, and S194 (d) the antibody or antigen-binding fragment thereof binds a residue of CD200R selected from T213 and E230:
(e) the antibody or antigen-binding fragment thereof does not bind to cynomolgus CD200RLa, or binds to cynomolgus CD200RLa with a K_{D} of more than 2 μM, as determined by surface plasmon resonance (SPR) at 37° C., optionally wherein the antibody or antigen-binding fragment thereof binds residues T213 and E230 of CD200R:
(f) the antibody or an antigen-binding fragment thereof specifically binds human CD200R or cynomolgus CD200R, wherein the antibody or antigen-binding fragment thereof does not bind to cynomolgus CD200RLa, or binds to cynomolgus CD200RLa with a KD of more than 2 μM, as determined by surface plasmon resonance (SPR) at 37° C., optionally wherein the antibody or antigen-binding fragment thereof binds a residue of CD200R selected from T213 and E230, optionally wherein the antibody or antigen-binding fragment thereof binds residues T213 and E230 of CD200R.

89.-98. (canceled)

99. The antibody of any claim 2, wherein
(a) the antibody is: (i) a monoclonal antibody; (ii) a human or humanized antibody; and/or (c) a chimeric antibody;
(b) the antibody or antigen-binding fragment thereof agonizes CD200R expressed on the surface of an immune cell;
(c) when binding to CD200R of an immune cell, the antibody or antigen-binding fragment thereof reduces activation of the immune cell relative to a comparable immune cell not bound by said antibody or antigen-binding fragment thereof, and/or
(d) when binding to CD200R of an immune cell, the antibody or antigen-binding fragment thereof decreases proliferation of the immune cell relative to a comparable immune cell not bound by said antibody or antigen-binding fragment thereof.

100.-102. (canceled)

103. The antibody or antigen-binding fragment thereof of claim 99, wherein
(a) said reduction in activation or proliferation of the immune cell is measured by an assay described in Example 5, 16, or 17,
(b) said decrease in cell proliferation or activation is measured in vitro or in vivo; and/or
(c) said decrease in cell proliferation or activation is at least about 10%, 15%, 20%, 25%, 30%, 40%, or 50%, or said decrease in cell proliferation or activation is from about 10% to 50%, 10% to 40%, 10% to 30%, 10% to 20%, 10% to 15%,20% to 50%,20% to 40%, or 20% to 30%.

104.-106. (canceled)

107. The antibody or antigen-binding fragment thereof of claim 2, wherein
(a) when binding to CD200R of an immune cell, the antibody or antigen-binding fragment thereof reduces expression of inflammatory genes in the immune cell relative to a comparable immune cell not bound by said antibody or antigen-binding fragment thereof;
(b) binding of said antibody or antigen-binding fragment thereof to CD200R expressed on the surface of an immune cell decreases NFκB signaling of said immune cell relative to a comparable immune cell not bound by said antibody or antigen-binding fragment thereof, optionally wherein said decrease in NFκB signaling of said immune cell is measured by an assay described in Example 5; and/or
(1) said decrease in NFκB signaling of said immune cell is at least about 10%, 15%, 20%, 25%, 30%, or 40%;
(2) said decrease in NFκB signaling of said immune cell is from about 10% to 40%, 10% to 30%, 10% to 20%, 20% to 40%, or 30% to 40%, or
(3) an average maximal percentage inhibition of NFκB signaling of said immune cell induced by the antibody or antigen-binding fragment thereof is at least 20%, 30%, 40%, 50%, or 60% greater than a control antibody, wherein the control antibody comprises:
(i) a control heavy chain comprising CDRH1, CDRH2, and CDRH3, which comprise amino acid sequences as set forth in SEQ ID NOs: 55-57, respectively, and a control light chain comprising CDRL1, CDRL2, and CDRL3, which comprise amino acid sequences as set forth in SEQ ID NOs: 58-60, respectively;
(ii) a control heavy chain variable region comprising the amino acid sequence as set forth in SEQ ID NO: 53, and a control light chain variable region comprising the amino acid sequence as set forth in SEQ ID NO: 54: or
(iii) a control heavy chain sequence comprising the amino acid sequence as set forth in SEQ ID NO: 73, and a control light chain sequence comprising the amino acid sequence as set forth in SEQ ID NO: 74:
(c) the immune cell is a T cell or a monocyte:
(d) said antibody or antigen-binding fragment thereof inhibits activation of basophils, optionally wherein said antibody or antigen-binding fragment thereof inhibits activation of basophils induced by FCεRI, or inhibits activation of basophils induced by binding of IgE to said basophils;
(e) said antibody or antigen-binding fragment thereof inhibits activation of basophils by
(1) at least 40% or at least 50%,
(2) about 10% to about 90%, about 20% to about 70%, about 30% to about 60%, or about 40% to about 60%, or
(3) about 10%, 20%, 30%, 40%, 45%, 50%, 55%, 60%, 70%, 80%, or 90%; and/or
(f) wherein said inhibition of activation of basophils is measured in an assay described in Example 18.

108.-120. (canceled)

121. The antibody or antigen-binding fragment thereof of claim 2, wherein
(a) the antibody or antigen-binding fragment thereof does not inhibit binding of CD200 to CD200R;
(b)
(i) said antibody or antigen-binding fragment thereof binds human CD200R with a KD of less than 10 nM, as determined by surface plasmon resonance (SPR) at 37° C.;
(ii) said antibody or antigen-binding fragment thereof binds human CD200R with a KD of less than 5 nM, as determined by surface plasmon resonance (SPR) at 37° C.;
(iii) said antibody or antigen-binding fragment thereof binds human CD200R with a KD of less than 2 nM, as determined by surface plasmon resonance (SPR) at 37° C.;
(iv) said antibody or antigen-binding fragment thereof binds human CD200R with a KD of less than 1 nM, as determined by surface plasmon resonance (SPR) at 37° C.;
(v) said antibody or antigen-binding fragment thereof binds human CD200R with a KD of less than 0.5 nM, as determined by surface plasmon resonance (SPR) at 37° C.;
(vi) said antibody or antigen-binding fragment thereof binds cynomolgus CD200R with a KD of less than 100 nM, as determined by surface plasmon resonance (SPR) at 37° C.:
(vii) said antibody or antigen-binding fragment thereof binds cynomolgus CD200R with a KD of less than 1 nM, as determined by surface plasmon resonance (SPR) at 37° C.:
(viii) said antibody or antigen-binding fragment thereof binds cynomolgus CD200R with a KD of less than 0.1 nM, as determined by surface plasmon resonance (SPR) at 37° C.; or
(ix) said antibody or antigen-binding fragment thereof binds cynomolgus CD200R with a K_{D} of less than 0.01 nM, as determined by surface plasmon resonance (SPR) at 37° C.:
(c) said antibody or antigen-binding fragment thereof does not induce significant cytokine release when said antibody or antigen-binding fragment thereof binds to CD200R on the surface of an immune cell:
(d) said antibody or antigen-binding fragment thereof comprises a domain that binds to an Fc receptor, optionally wherein said Fc receptor is expressed on the surface of an immune cell, optionally wherein said immune cell is an antigen presenting cell, optionally wherein said antigen presenting cell is a dendritic cell, macrophage, monocyte, or neutrophil: optionally wherein the binding of the antibody or antigen-binding fragment thereof to the Fc receptor expressed on the surface of the immune cell and binding to CD200R on the surface of a second immune cell results in the cell surface of the immune cell and the cell surface of the second immune cell to be within 250 Å, 200 Å, 150 Å, or 100 Å: and/or
said Fc receptor is FcγRIIB;
and/or
(e) the antibody or antigen-binding fragment thereof is bi-specific or multi-specific.

122.-131. (canceled)

132. An isolated nucleic acid that comprises one or more nucleotide sequences encoding polypeptides capable of forming the antibody or antigen-binding fragment thereof of claim 2.

133. A vector that comprises one or more nucleotide sequences encoding polypeptides capable of forming the antibody or antigen-binding fragment thereof of claim 2.

134. A host cell comprising one or more nucleic acid molecules encoding the amino acid sequence of a heavy chain and a light chain which when expressed are capable of forming the antibody or antigen-binding fragment thereof of any claim 2.

135. (canceled)

136. A method, comprising:
(a) providing a host cell comprising one or more nucleic acid molecules encoding the amino acid sequence of a heavy chain and a light chain which when expressed are capable of forming the antibody or antigen-binding fragment thereof of claim 2;
(b) culturing the host cell expressing the encoded amino acid sequence; and
(c) isolating the antibody or the antigen-binding fragment thereof.

137. An immunoconjugate comprising the antibody or antigen-binding fragment thereof of claim 2 conjugated with an agent.

138. A pharmaceutical composition comprising a therapeutically effective amount of the antibody or antigen-binding fragment thereof of claim 2 an immunoconjugate comprising the antibody or antigen-binding fragment thereof, and at least one pharmaceutically acceptable excipient.

139. (canceled)

140. A kit comprising the antibody or antigen-binding fragment thereof of claim 2, or an immunoconjugate comprising the antibody or antigen-binding fragment thereof or an pharmaceutical composition comprising the antibody, antigen-binding fragment thereof, or the immunoconjugate in a container; optionally further comprising a an informational material containing instructions.

141. (canceled)

142. (canceled)

143. A method of treating, preventing, alleviating, or reducing the severity of a disease or condition in a subject in need thereof, comprising administering to the subject a therapeutically effective amount of an antibody or antigen-binding fragment comprising a heavy chain variable region (HCVR) and a light chain variable region (LCVR), wherein:
(a) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 70, respectively, each with 0 to 3 amino acid modifications, such as 0, 1, 2, or 3 modifications; and the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications;
(b) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 3, 41, and 5, respectively, each with 0 to 3 amino acid modifications, such as 0, 1, 2, or 3 modifications; and the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 67, and 8, respectively, each with 0 to 3 amino acid modifications, such as 0, 1, 2, or 3 modifications;
(c) the HCVR comprises heavy chain complementarity determining region 1 (CDRH1), CDRH2, and CDRH3, and wherein CDRH1, CDRH2, and CDRH3 comprise the sequence as set forth in SEQ ID NOs: 92, 41, and 69, respectively; and the LCVR comprises light chain complementarity determining region 1 (CDRL1), CDRL2, and CDRL3, and wherein CDRL1, CDRL2, and CDRL3 comprise the sequence as set forth in SEQ ID NOs: 6, 87, and 91, respectively;
(d) the HCVR comprises an amino acid sequence having at least 90%, 95%, 96%, 97%, 98%, 99%, or 100% sequence identity to the amino acid sequence as set forth in SEQ ID NO: 72, and the LCVR comprises an amino acid sequence having at least 90%, 95%, 96%, 97%, 98%, 99%, or 100% sequence identity to the amino acid sequence as set forth in SEQ ID NO: 65;
(e) the HCVR comprises an amino acid sequence as set forth in SEQ ID NO: 72, with from 0 to 10 amino acid modifications, such as 1, 2, 3, 4, 5, 6, 7, 8, 9 or 10 amino acid modifications, and the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications;
(f) the HCVR comprises an amino acid sequence having at least 90%, 95%, 96%, 97%, 98%, 99%, or 100% sequence identity to the amino acid sequence as set forth in SEQ ID NO: 71, and the LCVR comprises an amino acid sequence having at least 90%, 95%, 96%, 97%, 98%, 99%, or 100% sequence identity to the amino acid sequence as set forth in SEQ ID NO: 65;
(g) the HCVR comprises an amino acid sequence as set forth in SEQ ID NO: 71, with from 0 to 10 amino acid modifications, such as 1, 2, 3, 4, 5, 6, 7, 8, 9 or 10 amino acid modifications, and the LCVR comprises an amino acid sequence as set forth in SEQ ID NO: 65, with from 0 to 10 amino acid modifications, such as 1, 2, 3, 4, 5, 6, 7, 8, 9 or 10 amino acid modifications;
(h) the HCVR comprises an amino acid sequence having at least 90%, 95%, 96%, 97%, 98%, 99%, or 100% sequence identity to the amino acid sequence as set forth in SEQ ID NO: 93, and the LCVR comprises an amino acid sequence having at least 90%, 95%, 96%, 97%, 98%, 99%, or 100% sequence identity to the amino acid sequence as set forth in SEQ ID NO: 94;
(i) the HCVR comprises an amino acid sequence as set forth in SEQ ID NO: 93, with from 0 to 10 amino acid modifications, such as 1, 2, 3, 4, 5, 6, 7, 8, 9 or 10 amino acid modifications, and the LCVR comprises an amino acid sequence as set forth in SEQ ID NO: 94, with from 0 to 10 amino acid modifications, such as 1, 2, 3, 4, 5, 6, 7, 8, 9 or 10 amino acid modifications;
(j) the antibody or antigen binding fragment thereof comprises a heavy chain comprising an amino acid sequence having at least 90%, 95%, 96%, 97%, 98%, 99%, or 100% sequence identity to the amino acid sequence as set forth in SEQ ID NO: 82, and a light chain comprising an amino acid sequence having at least 90%, 95%, 96%, 97%, 98%, 99%, or 100% sequence identity to the amino acid sequence as set forth in SEQ ID NO: 86;
(k) the antibody or antigen binding fragment thereof comprises a heavy chain comprising an amino acid sequence as set forth in SEQ ID NO: 82, with from 0 to 10 amino acid modifications, such as 1, 2, 3, 4, 5, 6, 7, 8, 9 or 10 amino acid modifications, and a light chain comprising an amino acid sequence as set forth in SEQ ID NO: 86, with from 0 to 10 amino acid modifications, such as 1, 2, 3, 4, 5, 6, 7, 8, 9 or 10 amino acid modifications;
(1) the antibody or antigen binding fragment thereof comprises a heavy chain comprising an amino acid sequence having at least 90%, 95%, 96%, 97%, 98%, 99%, or 100% sequence identity to the amino acid sequence as set forth in SEQ ID NO: 83, and a light chain comprising an amino acid sequence having at least 90%, 95%, 96%, 97%, 98%, 99%, or 100% sequence identity to the amino acid sequence as set forth in SEQ ID NO: 86;
(m) the antibody or antigen binding fragment thereof comprises a heavy chain comprising an amino acid sequence as set forth in SEQ ID NO: 83, with from 0 to 10 amino acid modifications, such as 1, 2, 3, 4, 5, 6, 7, 8, 9 or 10 amino acid modifications, and a light chain comprising an amino acid sequence as set forth in SEQ ID NO: 86, with from 0 to 10 amino acid modifications, such as 1, 2, 3, 4, 5, 6, 7, 8, 9 or 10 amino acid modifications;
(n) the antibody or antigen binding fragment thereof comprises a heavy chain comprising an amino acid sequence having at least 90%, 95%, 96%, 97%, 98%, 99%, or 100% sequence identity to the amino acid sequence as set forth in SEQ ID NO: 84, and a light chain comprising an amino acid sequence having at least 90%, 95%, 96%, 97%, 98%, 99%, or 100% sequence identity to the amino acid sequence as set forth in SEQ ID NO: 86;
(o) the antibody or antigen binding fragment thereof comprises a heavy chain comprising an amino acid sequence as set forth in SEQ ID NO: 84, with from 0 to 10 amino acid modifications, such as 1, 2, 3, 4, 5, 6, 7, 8, 9 or 10 amino acid modifications, and a light chain comprising an amino acid sequence as set forth in SEQ ID NO: 86, with from 0 to 10 amino acid modifications, such as 1, 2, 3, 4, 5, 6, 7, 8, 9 or 10 amino acid modifications;
(p) the antibody or antigen binding fragment thereof comprises a heavy chain comprising an amino acid sequence having at least 90%, 95%, 96%, 97%, 98%, 99%, or 100% sequence identity to the amino acid sequence as set forth in SEQ ID NO: 85, and a light chain comprising an amino acid sequence having at least 90%, 95%, 96%, 97%, 98%, 99%, or 100% sequence identity to the amino acid sequence as set forth in SEQ ID NO: 86;
(q) the antibody or antigen binding fragment thereof comprises a heavy chain comprising an amino acid sequence as set forth in SEQ ID NO: 85, with from 0 to 10 amino acid modifications, such as 1, 2, 3, 4, 5, 6, 7, 8, 9 or 10 amino acid modifications, and a light chain comprising an amino acid sequence as set forth in SEQ ID NO: 86, with from 0 to 10 amino acid modifications, such as 1, 2, 3, 4, 5, 6, 7, 8, 9 or 10 amino acid modifications;
(r) the antibody or antigen binding fragment thereof comprises a heavy chain comprising an amino acid sequence having at least 90%, 95%, 96%, 97%, 98%, 99%, or 100% sequence identity to the amino acid sequence as set forth in SEQ ID NO: 95, and a light chain comprising an amino acid sequence having at least 90%, 95%, 96%, 97%, 98%, 99%, or 100% sequence identity to the amino acid sequence as set forth in SEQ ID NO: 98;
(s) the antibody or antigen binding fragment thereof comprises a heavy chain comprising an amino acid sequence as set forth in SEQ ID NO: 95, with from 0 to 10 amino acid modifications, such as 1, 2, 3, 4, 5, 6, 7, 8, 9 or 10 amino acid modifications, and a light chain comprising an amino acid sequence as set forth in SEQ ID NO: 98, with from 0 to 10 amino acid modifications, such as 1, 2, 3, 4, 5, 6, 7, 8, 9 or 10 amino acid modifications;
(t) the antibody or antigen binding fragment thereof comprises a heavy chain comprising an amino acid sequence having at least 90%, 95%, 96%, 97%, 98%, 99%, or 100% sequence identity to the amino acid sequence as set forth in SEQ ID NO: 96, and a light chain comprising an amino acid sequence having at least 90%, 95%, 96%, 97%, 98%, 99%, or 100% sequence identity to the amino acid sequence as set forth in SEQ ID NO: 98;
(u) the antibody or antigen binding fragment thereof comprises a heavy chain comprising an amino acid sequence as set forth in SEQ ID NO: 96, with from 0 to 10 amino acid modifications, such as 1, 2, 3, 4, 5, 6, 7, 8, 9 or 10 amino acid modifications, and a light chain comprising an amino acid sequence as set forth in SEQ ID NO: 98, with from 0 to 10 amino acid modifications, such as 1, 2, 3, 4, 5, 6, 7, 8, 9 or 10 amino acid modifications;
(v) the antibody or antigen binding fragment thereof comprises a heavy chain comprising an amino acid sequence having at least 90%, 95%, 96%, 97%, 98%, 99%, or 100% sequence identity to the amino acid sequence as set forth in SEQ ID NO: 97, and a light chain comprising an amino acid sequence having at least 90%, 95%, 96%, 97%, 98%, 99%, or 100% sequence identity to the amino acid sequence as set forth in SEQ ID NO: 98; or
(w) the antibody or antigen binding fragment thereof comprises a heavy chain comprising an amino acid sequence as set forth in SEQ ID NO: 97, with from 0 to 10 amino acid modifications, such as 1, 2, 3, 4, 5, 6, 7, 8, 9 or 10 amino acid modifications, and a light chain comprising an amino acid sequence as set forth in SEQ ID NO: 98, with from 0 to 10 amino acid modifications, such as 1, 2, 3, 4, 5, 6, 7, 8, 9 or 10 amino acid modifications.

144.-153. (canceled)

154. The method of claim 143, wherein administering the antibody or antigen-binding fragment thereof comprises
(i)parenteral, intravenous, oral, subcutaneous, intra-arterial, intracranial, intrathecal, intraperitoneal, intratumoral, topical, intranasal or intramuscular administration, or
(ii) intravenous, subcutaneous, or intramuscular administration.

155.-157. (canceled)

158. The method of claim 143, wherein the disease or condition comprises
(a) disease or condition associated with CD200R activity or function, or
(b) an autoimmune disease or condition or an inflammatory disease or condition, optionally wherein the inflammatory disease or condition is selected from a rheumatological disease or condition, gastrointestinal disease or condition, pulmonary disease or condition, hepatological disease or condition, nephrological disease or condition, and dermatological condition, optionally wherein the inflammatory disease or condition is
(i) a rheumatological disease or condition selected from rheumatoid arthritis (RA), systemic lupus erythematosus (SLE), lupus nephritis (LN), osteoarthritis (OA), Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, Takayasu arteritis, cutaneous lupus erythematosus (CLE) types: chronic CLE (including discoid), subacute CLE, acute CLE, seropositive or seronegative RA, juvenile idiopathic arthritis (JIA), primary OA or secondary OA, cervical and lumbar spinal OA, hip OA, knee OA, and erosive OA, or
(ii) a rheumatological disease or condition selected from rheumatoid arthritis (RA), systemic lupus erythematosus (SLE), lupus nephritis (LN), and osteoarthritis (OA).

159.-162. (canceled)

163. The method of claim 158, wherein the inflammatory disease or condition is
(a) a rheumatological disease or condition that is rheumatoid arthritis (RA), optionally further comprising co-administering
(1) one or more additional therapeutic agents selected from disease-modifying antirheumatic drugs (DMARDS), such as hydroxychloroquine, sulfasalazine, methotrexate, and leflunomide: TNF inhibitors (e.g., etanercept, adalimumab, infliximab, golimumab, certolizumab pegol), T cell costimulatory inhibitor, (e.g., abatacept), IL-6 receptor inhibitors (e.g., tocilizumab, sarilumab), anti-CD20 antibody (e.g., rituximab); and JAK inhibitors (e.g., tofacitinib, baricitinib, upadacitinib): NSAIDs, such as ibuprofen, naproxen, and diclofenac; COX-2 inhibitor, such as celecoxib and etoricoxib; steroids and corticosteroids, such as prednisolone and cortisone; and biological agents known for treatment and/or prophylaxis of such conditions, including for example etanercept (e.g., ENBREL), infliximab (e.g., REMICADE), adalimumab (e.g., HUMIRA), anakinra (e.g., KINARET), abatacept (ORENCIA), rituximab (e.g., RITUXAN), certolizumab (e.g., CIMZIA), golimumab (e.g., SIMPONI), and tocilizumab (e.g., ACTEMRA), and/or
(2) two additional therapeutic agents, such as (i) methotrexate and leflunomide; (ii) methotrexate and sulfasalazine: (iii) methotrexate and cyclosporine; or (iv) methotrexate and hydroxvchloroquine; or three additional therapeutic agents, such as (i) hvdroxvchloroquine, sulfasalazine and methotrexate; or (ii) hvdroxvchloroquine, sulfasalazine, and leflunomide;
(b) a rheumatological disease or condition that is systemic lupus erythematosus (SLE), optionally further comprising co-administering
(1) one or more additional therapeutic agents selected from hydroxvchloroquine, steroids and corticosteroids (e.g., prednisone, methylprednisolone), belimumab, azathioprine, methotrexate, cyclophosphamide, mycophenolate and mycophenolate mofetil, cyclosporine, leflunomide, voclosporin, abatacept, anifrolumab, rituximab, NSAIDS, such as naproxen sodium and ibuprofen, antimalarial drugs, such as hydroxvchloroquine, calcineurin inhibitors, and tacrolimus, or
(2) one or more additional therapeutic agents selected from nonsteroidal anti-inflammatory drugs (NSAIDs), topical capsaicin, intraarticular glucocorticoid injections, acetaminophen, duloxetine, tramadol, and injectable corticosteroids such as methylprednisolone acetate, triamcinolone acetate, betamethasone acetate and betamethasone sodium phosphate, triamcinolone hexacetonide, and dexamethasone:
(c) a rheumatological disease or condition that is lupus nephritis (LN), optionally further comprising co-administering
(1) one or more additional therapeutic agents selected from azathioprine, belimumab, cyclophosphamide, cyclosporine, mycophenolic acid analogs, mizoribine, mycophenolic acid sodium, prednisone, rituximab, tacrolimus, and voclosporin,
(2) two additional therapeutic agents, such as (i) prednisone and mycophenolic acid analogs: (ii) prednisone and mycophenolic acid sodium; (iii) prednisone and cyclophosphamide; (iv) prednisone and tacrolimus; (v) prednisone and voclosporin; (vi) prednisone and rituximab; (vii) prednisone and azathioprine: (viii) prednisone and cyclosporine; or (ix) prednisone and mizoribine, or
(3) three additional therapeutic agents, such as (i) prednisone, belimumab, and mycophenolic acid analogs: or (ii) prednisone, belimumab, and cyclophosphamide;
or
(d) a rheumatological disease or condition that is osteoarthritis.

164.-174. (canceled)

175. The method or antibody or antigen-binding fragment thereof of claim 158, wherein the inflammatory disease or condition is
(a) a gastrointestinal disease or condition selected from ulcerative colitis (UC), Crohn's disease (CD), eosinophilic gastrointestinal disorders (EGIDs), microscopic colitis, ulcerative proctitis, proctosignmoiditis, left-sided colitis, extensive colitis, pancolitis, ileocolitis, ileitis, gastroduodenal CD, jejunoileitis, and Crohn's (granulomatous) colitis, and/or wherein the inflammatory disease or condition is a gastrointestinal disease or condition selected from ulcerative colitis (UC) and Crohn's disease (CD), optionally further comprising co-administering one or more additional therapeutic agents selected from infliximab, adalimumab, golimumab, vedolizumab, tofacitinib, ustekinumab, natalizumab, mesalamine, diazo-bonded 5-ASA, sulfasalazine, balsalazide, olsalazine, corticosteroids such as budesonide, hydrocortisone, methylprednisolone, and prednisone; immunosuppressants or immunomodulators such as azathioprine and 6-mercaptopurine, cyclosporine, and methotrexate:
(b) a pulmonary disease or condition selected from idiopathic pulmonary fibrosis (IPF), interstitial lung disease (ILD), acute respiratory distress syndrome (ARDS), asthma, bronchiolitis obliterans, chronic obstructive pulmonary disease (COPD), Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis, bronchiectasis, primary ciliary dyskinesia, pneumonia pulmonary arterial hypertension (PAH), lymphangioleiomyomatosis, nonspecific interstitial pneumonia, cryptogenic organizing pneumonia, acute interstitial pneumonia, familial interstitial lung disease, and bleomycin induced pulmonary fibrosis, optionally wherein the inflammatory disease or condition is a pulmonary disease or condition selected from idiopathic pulmonary fibrosis (IPF) and interstitial lung disease (ILD), and/or optionally further comprising co-administering one or more additional therapeutic agents selected from nitendanib, pirfenidone, corticosteroids such as prednisone, other rheumatologic drugs, including mycophenolate (e.g., CellCept®), azathioprine (e.g., Imuran®), leflunomide (e.g., ARAVA®), rituximab (e.g., RITUXAN®), cyclophosphamide (e.g., CYTOXAN®), tacrolimus (e.g., PROGRAF®), medications that reduce stomach acid, such as H-2-receptor antagonists or proton pump inhibitors such as lansoprazole (e.g.. PREVACID®24HR), omeprazole (e.g., Prilosec OTC) and pantoprazole (e.g., PROTONIX®):
(c)
(i) a hepatological disease or condition selected from non-alcoholic steatohepatitis (NASH), primary sclerosing cholangitis (PSC), primary biliary cirrhosis (PBC), autoimmune hepatitis, alcoholic steatohepatitis (ASH), alcoholic hepatitis, chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), and Barrett's esophagus:
(ii) a hepatological disease or condition selected from non-alcoholic steatohepatitis (NASH) and Non-Alcoholic Fatty Liver Disease (NAFLD): or
(iii) a nephrological disease or condition selected from diabetic kidney disease (DKD) (diabetic nephropathy), chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 diabetes mellitus, type 2 diabetes mellitus, and hyperuricemia:
(d) a nephrological disease or condition selected from diabetic kidney disease (DKD) (diabetic nephropathy) and chronic kidney disease (CKD): and/or wherein the further comprising co-administering one or more additional therapeutic agents selected from metformin, sodium-glucose cotransporter-2 inhibitor (SGLT2i), drug therapy for glycemic control, DPP-4 inhibitor, insulin, sulfonylurea, TZD (thiazolidinedione), alpha-glucosidase inhibitor, SGLT2 inhibitor (e.g., empagliflozin, canagliflozin, dapaglifloz), glucagon-like peptide-1 receptor agonist (GLP-1 RA) (e.g., lixisenatide, liraglutide, semaglutide, exenatide, albiglutide, dulaglutide), DPP-4 inhibitors (e.g., saxagliptin, alogliptin, sitagliptin, linagliptin), one or more agents used to treat high blood pressure such as angiotensin-converting enzyme (ACE) inhibitors and angiotensin 2 receptor blockers (ARBs), agents supportive of weight loss or for control of blood sugar, cholesterol-lowering drugs (e.g., statins), finerenone, and agents for treatment of diabetes mellitus, such as alpha-glucosidase inhibitors (e.g., acarbose, miglitol, voglibose):
or
(d)
(1) a dermatological condition selected from atopic dermatitis (AD), contact dermatitis, dyshidrotic eczema, seborrheic dermatitis, neurodermatitis, nummular eczema, stasis dermatitis, hand eczema, vitiligo, alopecia areata, acne, psoriasis, dermatomyositis, scleroderma, and morphea; or a dermatological condition that is atopic dermatitis; and/or
(2) optionally further comprising co-administering one or more additional therapeutic agents selected from topical corticosteroids (TCS) (e.g., desonid, hydrocortisone, fluocinolone, triamcinolone, betamethasone diproprionate), topical calcineurin inhibitors (TCI) (e.g., tacrolimus, pimecrolimus), topical antimicrobials and antiseptics, cyclosporine, methotrexate, mycophenolate mofetil, interferon gamma, phosphodiesterase 4 (PDE4) inhibitor such as crisaborole, JAK inhibitor (e.g., ruxolitinib, upadacitinib, abrocitinib), systemic glucocorticoids (e.g., prednisone), dupilumab, and anti-IL-13 antibody (e.g., tralokinumab), or wherein the disease or condition comprises a dermatological disease or condition and the method optionally further comprises administering to the subject one or more additional therapeutic agents selected from antihistamines, corticosteroids, calcineurin inhibitors, antibiotics, and light therapy: optionally wherein
(i) the antihistamines is selected from diphenhydramine, cetirizine, desloratadine, fexofenadine, levocetirizine, and loratadine, or
(ii) the corticosteroid is selected from cortisone, hydrocortisone, and prednisone, and/or wherein the corticosteroid is administered as a cream, ointment, or orally,
(iii) the calcineurin inhibitor is selected from astagraf xl, cequa, cyclosporine, cyclosporine ophthalmic, elidel, envarsus xr, gengraf, hecoria, lupkynis, neoral, pimecrolimus, prograf, protopic, restasis, sandimmune, tacrolimus, tacrolimus ointment, verkazia, and voclosporin, or
(iv) the antibiotic is selected from vancomycin, ceftaroline, daptomycin, doxycycline, linezolid, telavancin, tigecycline, and trimethoprim-sulfamethoxazole.

176.-194. (canceled)

195. The method of claim 143, wherein the disease or condition comprises an autoimmune skin disease or condition, optionally wherein the autoimmune skin disease or condition comprises Behcet's disease, dermatitis herpetiformis, dermatomyositis, epidermolysis bullosa, lichen planus, linear IgA disease, lupus of the skin, morphea/scleroderma, ocular cicatrical pemphigoid, pemphigoid, bullous pemphigoid, pemphigus, psoriasis, scleroderma, or vasculitis; and/or
optionally further comprising administering to the subject 1, 2, 3, 4, or more additional therapeutic agents, optionally wherein the one or more additional therapeutic agents is selected from 5-HT 1a receptor partial agonists and antagonists, 5-HT 2a receptor partial agonists and antagonists, 5-HT 2b receptor antagonists, 5-HT 6 receptor antagonists, 5-HT 7 receptor antagonists, Abl tyrosine kinase inhibitors, ACE inhibitors, Acidic mammalian chitinase inhibitors, Actin antagonists, Acetaldehyde dehydrogenase inhibitors, Acetyl CoA carboxylase (ACC) inhibitors, ACC-1 inhibitors, ACC-2 inhibitors, 2-Acylglycerol O-acyltransferase 2 (DGAT2) inhibitors, ACTH receptor agonists, Activin receptor antagonists, Adenosylhomocysteinase inhibitors, Adenosine receptor antagonists and agonists, Adenosine deaminase inhibitors, Adenylyl cyclase associated protein 1 inhibitors, Adiponutrin inhibitors, Adiponectin receptor agonists, ADP ribosyl cyclase-1 inhibitors, ADP ribosyl cyclase-1 modulators, ADP ribosylation factor 6 inhibitors, Adrenocorticotrophic hormone ligands, adrenomedullin ligands, Adrenergic receptor antagonists and agonists, Adropin stimulators, Aggrecanase-2 inhibitors, AIMP multisynthetase complex protein 1 stimulators, AKT1 gene inhibitors, AKT protein kinase inhibitors, Albumin antagonists, Albumin modulators, Aldehyde dehydrogenase 2 stimulators, Aldosterone antagonists, Aldosterone synthase inhibitors Alk-5 protein kinase inhibitors, Alpha 2 adrenoceptor agonists, Alpha 2 adrenoceptor modulators, Alpha 1 antitrypsin stimulator, alpha-fetoprotein modulators, Alstrom syndrome protein 1(ALMS1)/PKC alpha protein interaction inhibitors, 1 Aminocyclopropane carboxyl synthase inhibitors, Amylin receptor agonists, AMP-activated protein kinases (AMPK), AMP activated protein kinase inhibitors, activators or stimulators, AMP activated protein kinase alpha 2 stimulators, Androgen receptor agonists and antagonists, Angiopoietin-related protein-3 inhibitors, Angiotensin II receptor antagonists, Angiotensin II AT-1 receptor antagonists, Angiotensin II AT-2 receptor agonists, Angiotensinogen ligand inhibitors, Annexin Al modulators, antibiotics, antifungals, anti-TL6 antibodies, anti-TNF steroid conjugates, activator protein 1 (AP1) transcription factor inhibitors, AP1 transcription factor modulators, Apelin receptor agonists, APOA1 gene stimulators, Apolipoprotein A antagonists, Apolipoprotein B modulators, Apolipoprotein L1 modulators, Apoptosis regulator Bcl w inhibitors, Aryl hydrocarbon receptor (AHR) agonists and modulators, AHR agonist plus autoantigen, ASK1 inhibitors, ATPase inhibitors, ATP binding cassette transporter C2 inhibitors, ATP citrate lyase inhibitors, Autophagy protein modulators and stimulators, Autotaxin inhibitors, Axl tyrosine kinase receptor inhibitors, BAFF/APRIL inhibitors, Basigin inhibitors, B and T lymphocyte attenuator stimulators, Bax protein stimulators, Bcl-2 protein inhibitors, Bcl-xL Bcl-2 associated death promotor inhibitors, Bcl-xL Bcl-2 associated death promotor modulators, Bcr protein inhibitors, Benzodiazepine receptor agonists, beta adrenoceptor antagonists, BET inhibitors, Beta 2 adrenoceptor agonists, Beta amyloid antagonists, Beta-catenin inhibitors, Beta-catenin modulators, Beta-catenin stimulators, Beta-galactosidase inhibitors, beta lactamase modulators, 17 beta hydroxysteroid dehydrogenase 13 inhibitors, Bifunctional aminoacyl tRNA synthetase inhibitors, B-lymphocyte antigen CD19 inhibitors, B-lymphocyte antigen CD20 inhibitors, B-lymphocyte antigen CD20 modulators, B-lymphocyte cell adhesion molecule inhibitors, B-lymphocyte stimulator ligand inhibitors, B-lymphocyte stimulator ligand modulators, Bioactive lipids, Bone morphogenetic protein-7 ligand, Bone morphogenetic protein-7 ligand modulators, Bradykinin receptor modulators, BRAF gene inhibitors, Branched amino acid aminotransferase 1 inhibitors, Bromodomain containing protein (BRD) inhibitors, BRD1, BRD2, and BRD4 inhibitors, BTK inhibitors, B7 homolog inhibitor, Cadherin-11 antagonists, Cak tyrosine kinase receptor inhibitors, Calcineurin inhibitors, Calcium channel inhibitors, Ca2+release activated Ca2+channel 1 inhibitors, Calcitonin agonists, Calpain-IX inhibitors, Calpain-I inhibitors, Calpain-II inhibitors, Calreticulin inhibitors, Caveolin 1 stimulators, Cannabinoid CB1 receptor antagonists and inverse agonists, Cannabinoid CB2 receptor agonists, Cannabinoid receptor antagonists and agonists, Cannabinoid CB1 receptor inverse agonists, carbohydrate metabolism modulators, Carbonic anhydrase inhibitors, Casein kinase-I delta and/or epsiloninhibitors, CASP9 gene stimulators, Caspase inhibitors, Caspase-3 stimulators, Catalase stimulators, Cathepsin inhibitors, Cathepsin K inhibitors, Cathepsin S inhibitors, Caveolin 1 inhibitors, CCK receptor antagonists, CCAAT enhancer binding protein beta modulators, C-C motif ligand 26 (CCL26) gene inhibitors, Chemokine receptor antagonists, C-C motif chemokine receptor (CCR) 1 antagonists, CCR2 antagonists, CCR3 antagonists and modulators, CCR4 antagonists, CCR5 antagonists, CCR6 antagonists, CCR7 modulators, CCR9 chemokine antagonists, CCR3 gene modulators, CD3 modulators or antagonists, CD4 agonists or antagonists, CD7 inhibitors, CD11b agonists, CD29 modulators, CD39 agonists, CD40 ligand receptor modulators or antagonists, CD47 antagonists, CD52 antagonists, CD73 agonists and antagonists, CD79b modulators, CD80 modulators or antagonists, CD86 modulators or antagonists, CD95 antagonists, CD126 antagonists, CD223 modulators, CDGSH iron sulfur domain protein modulators, CDwl23 antagonists, Cell adhesion molecule inhibitors, Cell surface glycoprotein CD200R agonists, Cell surface glycoprotein MUC18 inhibitors, chemokine CXC ligand inhibitors, Chaperonin inhibitors and modulators, chitinase inhibitors, Chitotriosidase 1 inhibitors, Chloride channel stimulators, Cholera enterotoxin subunit B inhibitors, Choline kinase inhibitors, CHST15 gene inhibitors, Chymase inhibitors, Claudin 1 inhibitors, Clusterin stimulators, CNR1 inhibitors, Collagen I antagonists, Collagen VII antagonists, Collagen gene inhibitors, Collagenase inhibitors, collagen modulators, Complement Clq subcomponent inhibitors, Complement Cis subcomponent inhibitors, Complement C3 inhibitors, Complement C5 factor inhibitors, Complement C5a receptor antagonists, Complement cascade inhibitors, Complement Factor stimulators, Complement Factor B inhibitors, Complement factor D inhibitors, Connective tissue growth factor ligand inhibitors, Corticosteroid hormone receptor agonists, COT protein kinase inhibitors, CREB binding protein inhibitors, C-reactive protein (CRP) inhibitors, cerebrospinal fluid (CSF)-1 agonists and antagonists, C-type lectin domain protein 4C inhibitors, CTGF gene inhibitors, CX3CR1 antagonists and modulators, CXCR2 antagonists, CXCR3 antagonist, CXCR4 antagonists and modulators, CXCR5 antagonists and modulators, CXC5 ligand inhibitors, CXC6 chemokine ligand inhibitors, CXC10 ligand inhibitors, CXC11 ligand modulators, Cyclin-dependent kinase (CDK) 1, 2, 5, 7, and/or 9 inhibitors, Cyclooxygenase (COX) inhibitors, COX-1 inhibitors, COX-2 inhibitors and modulators, Cysteine palmitoyltransferase porcupine inhibitors, Cytochrome P450 7A1 inhibitors, Cytochrome P450 11B2 inhibitors, Cytochrome P450 2E1 inhibitors (CYP2E1), Cytochrome P450 reductase inhibitors, Cytokine receptor agonists and antagonists, Cytosolic phospholipase A2 (cPLA2) inhibitors, Cytotoxic T-lymphocyte protein-4 (CTLA4) modulators and stimulators, Deoxyribonuclease (DNase) modulators, DNase gamma stimulators, DNase I stimulators, DGAT2 gene inhibitors, DHFR inhibitors, Diacylglycerol O acyltransferase (DGAT) 1 inhibitors, DGAT2 inhibitors, Diamine acetyltransferase inhibitors, Dihydroceramide delta 4 desaturase inhibitors, Dihydroorotate dehydrogenase inhibitors, Dipeptidyl peptidase (DPP)I inhibitors, DPP IV inhibitors, DNA binding protein Ikaros inhibitors, DNA methyltransferase inhibitors, DNA polymerase inhibitors, Dopamine D2 receptor partial agonists, Dopamine D3 receptor partial agonists, Dopamine D4 receptor partial agonists, Dopamine D2 receptor agonists, DYRK-1 alpha protein kinase inhibitors, Ectonucleotide pyrophosphatase-PDE-2 inhibitors, EGFR tyrosine kinase receptor inhibitors, EGR1 gene inhibitors, Elongation factor 2 inhibitors, Endoglin inhibitors, Endoplasmin inhibitors, Endosialin modulators, Endostatin modulators, Endothelin ET-A receptor antagonists, Endothelin ET-B receptor antagonists, Endothelial nitric oxide synthase stimulators, Enolase 1 inhibitors, Enteropeptidase inhibitors, Eotaxin 2 ligand inhibitors, eotaxin ligand inhibitors, EP4 prostanoid receptor antagonists or agonists, EP4 prostanoid receptor antagonists, Epidermal growth factor (EGF) receptor antagonists, EGF modulators, Epoxide hydrolase inhibitors, Erythropoietin receptor antagonists or agonists, Exportin 1 inhibitors, Extracellular matrix protein modulators, FIFO ATP synthase modulators, Facilitated glucose transporter-1 modulators, Factor IIa antagonists, Factor XIIa antagonists, Farnesoid X receptor (FXR) agonists and modulators, Fatty acid synthase inhibitors, fecal microbiota transplantations (FMT), fibroblast activation protein (FAP) inhibitors, Fibroblast growth factor (FGF) receptor agonists and antagonists, FGF-2 ligand inhibitors, FGF1 receptor agonists and antagonists, FGF2 receptor antagonists, FGF3 receptor antagonists, FGF19 gene stimulators, FGF-15 ligands or modulators, FGF-19 ligands or modulators, FGF-21 ligands or modulators, FK506 binding protein inhibitors, FK506 binding protein-10 inhibitors, FK506 binding protein-12 modulators, Flt3 tyrosine kinase inhibitors, Focal adhesion kinase inhibitors, Folate antagonists or agonists, Folate receptor beta antagonists, FP prostanoid receptor antagonists, Fractalkine ligand inhibitors, Free fatty acid receptor 1, 2, and/or 3 agonists, free fatty acid receptor 2 antagonists, Frizzled-5 receptor agonists, Frizzled-8 receptor agonists, Fyn tyrosine kinase inhibitors, G-protein coupled bile acid receptor 1 agonists, G protein coupled receptor 15 antagonists, G-protein beta subunit inhibitors, G-protein coupled receptor (GPCR) 35, 44, 84, 119, 120 modulators, GPCR 44, 87 antagonists, GABA A receptor modulators, GABA A receptor alpha-2 subunit modulators, GABA A receptor alpha-3 subunit modulators, Galanin GAL2 receptor agonists, Galectin-3 inhibitors, Gastric inhibitory polypeptide receptor (GIP-R) agonists and modulators, GATA 3 transcription factor inhibitors, GDNF family receptor alpha like agonists, GHR gene inhibitors, Glucagon-like peptide (GLP) 1 agonists, GLP 2 agonists, GLP 1 receptor modulators, Glucocorticoid agonists or antagonists, Glucocorticoid induced leucine zipper stimulators, Glucokinase stimulators, Glucose 6-phosphate 1-dehydrogenase inhibitors, Glutaminyl peptide cyclotransferase inhibitors, Glutaredoxin 1 modulators, Glutathione dependent PGD synthase inhibitors, Glycoprotein 1b (GPIb) antagonists, GM-CSF receptor antagonists or modulators, GMP synthetase inhibitors, GNRH receptor modulators, GP IIb IIIa antagonists, GPCR modulators, GPR40 agonists, GPR84 antagonists, GroEL protein 2 inhibitors, GroEL protein 2 inhibitors, Growth hormone ligands, Growth hormone receptor agonists, Growth regulated protein alpha ligand inhibitors, guanylate cyclase receptor agonists, Guanylate cyclase stimulators, Heat shock protein inhibitors, H+K+ATPase inhibitors, Hedgehog (Hh) modulators, Hh protein inhibitors, Heme oxygenase 1 modulators, Hepatitis B structural protein inhibitors, Hepatitis C virus NS3 protease inhibitors, Hepatitis C virus protein NS5A inhibitors, Hepatocyte nuclear factor 4 alpha modulators (HNF4A), Hepatocyte growth factor modulators and antagonists, hypoxia inducible factor (HIF) prolyl hydroxylase inhibitors, HIF prolyl hydroxylase-2 inhibitors, High mobility group protein B1 inhibitors, Histamine H1 receptor antagonists, Histamine H4 receptor agonists, Histamine H4 receptor antagonists, Histamine H4 receptor modulators, Histone deacetylase (HDAC) inhibitors, HDAC-1 inhibitors, HDAC-2 inhibitors, HDAC-3 inhibitors, HDAC-6 inhibitors, H+K+ATPase inhibitors, HIV-1 gp120 protein inhibitors, HLA antigen modulators, HLA class II antigen DQ-2 alpha modulators, HLA class II antigen DR-1 beta inhibitors, HLA class II antigen inhibitors, HLA class II antigen modulators, HMG CoA reductase inhibitors, Homeodomain interacting kinase 2 (HIPK2) inhibitors, Hormone sensitive lipase stimulators, HSD17B3 gene modulators, HSD17B13 gene inhibitors, Hsp 70 family inhibitors and stimulators, Hsp 90 inhibitors, Hvaluronidase stimulators, Hydrolase inhibitors, Hypoxia inducible factor (HIF) modulators, HIF-1 inhibitors, HIF-1 alpha modulators and stimulators, HIF-2 alpha inhibitors, ICAM1 gene inhibitors, ICE inhibitors, interferon beta (IFNB) gene stimulators, Insulin-like growth factor 1 (IGF1) gene inhibitors, IgG receptor FcRn large subunit p51 antagonists, IgG receptor FcRn large subunit p51 modulators, I-kappa B kinase inhibitors, I-kappa B kinase beta inhibitors, IK potassium channel inhibitors, Interleukin (IL)-1 antagonists, IL-2 agonists or antagonists, IL-3 antagonists, IL-4 agonists or antagonists, IL-5 antagonists, IL-6 agonists or antagonists, IL-7 receptor antagonists, IL-8 antagonists, 10 antagonists or agonists, IL-11 agonists, IL-12 antagonists, IL-13 antagonists, IL-15 antagonists, IL-17, IL17 Å, and IL17B agonists or antagonists, IL-18 antagonists, IL-21 antagonists, IL-22 agonists or antagonists, IL-23 antagonists, IL-1 beta ligand modulators, IL-23A inhibitors, IL-31 receptor modulators and antagonists, IL-36 inhibitors, IL-6 neutralizing human antibodies, IL-1 receptor accessory protein inhibitors, IL-18 receptor accessory protein antagonists, IL-2 receptor alpha subunit inhibitors, IL-2 receptor alpha subunit stimulators, Interleukin ligands, IL-1 alpha ligand inhibitors, IL-1 ligand inhibitors, IL-1 beta ligand inhibitors and modulators, IL-1 beta ligands, interleukin ligand inhibitors, IL-2 ligands, IL-4 ligands, IL-4 ligand inhibitors, IL-6 ligand inhibitors, IL-8 ligand inhibitors, IL-10 ligands, IL-13 ligand inhibitors, IL 17 ligand inhibitors, IL 17A ligand inhibitors and modulators, IL-17F ligand inhibitors, IL 18 ligand inhibitors, Interleukin-22 ligands, IL-29 ligands, IL-33 ligand inhibitors, IL-1 like receptor inhibitors, Ileal sodium bile acid cotransporter inhibitors, immunoglobulin (Ig) agonists or antagonists, IgE antagonists and modulators, Immunoglobulin Fc receptor modulators, IgG agonists, IgGI agonists and antagonists, IgG2 antagonists and modulators, Immunoglobulin gamma Fc receptor antagonists, Immunoglobulin gamma Fc receptor II modulators, Immunoglobulin gamma Fc receptor IIB antagonists, Immunoglobulin kappa modulators, Immunoglobulin like domain receptor 2 antagonists, IgM antagonists, Inducible nitric oxide synthase inhibitors (iNOS inhibitors), Inducible T-cell co-stimulator inhibitors, Inosine monophosphate dehydrogenase inhibitors, Insulin ligands, Insulin ligand agonists, Insulin receptor agonists, Insulin receptor substrate-1 inhibitors, Insulin sensitizers, integrin antagonists and modulators, Integrin alpha-1/beta-1 antagonists, Integrin alpha-4/beta-1 antagonists, Integrin alpha-V/beta-1 antagonists, Integrin alpha-V/beta-3 antagonists, Integrin alpha-V/beta-6 antagonists, Integrin alpha-V/beta-8 modulators, integrin alpha-4/beta-7 antagonists, Integrin alpha-9 antagonists, Interferon (IFN) alpha ligands, IFN alpha ligand inhibitors and modulators, IFN omega ligand inhibitors, IFN beta ligands, IFN beta ligand inhibitors, IFN gamma ligands, IFN gamma receptor 1 agonists, IFN gamma receptor antagonists, IFN type I receptor antagonists, interleukin-1 receptor-associated kinase 4 (IRAK4) inhibitors, IRE1 protein kinase inhibitors, Itk tyrosine kinase inhibitors, Janus Kinase (JAK) inhibitors and modulators, JAK3 gene inhibitors, JAK1 inhibitors, JAK2 inhibitors, JAK3 inhibitors, Jun N terminal kinase inhibitors, Jun N terminal kinase-1 inhibitors, Kallikrein inhibitors, Kallikrein 2 inhibitors, Kallikrein 7 inhibitors, KCNA voltage-gated potassium channel-3 inhibitors, KCNA voltage-gated potassium channel-3 modulators, KCNN potassium channel-4 inhibitors, KCNN4 gene inhibitors, Kelch like ECH associated protein 1 modulators, Ketohexokinase (KHK) inhibitors, Kit tyrosine kinase inhibitors, Klotho beta stimulators, lactoferrin stimulators, LanC like protein 2 stimulators, LanC like protein 2 modulators, Lck tyrosine kinase inhibitors, LDHA gene inhibitors, LDL receptor related protein-1 stimulators, LDL receptor related protein-6 inhibitors, LDL receptor related protein-6 stimulators, Lectin mannose binding protein inhibitors, leukocyte elastase inhibitors, Leukocyte Ig like receptor A4 modulators, leukocyte proteinase-3 inhibitors, Leukotriene receptor antagonists, Leukotriene A4 hydrolase inhibitors, Leukotriene BLT receptor antagonists, Leukotriene D4 antagonists, 5-Lipoxygenase activating protein inhibitors, 5-Lipoxygenase inhibitors, Lipoxygenase modulators, Lipoprotein lipase inhibitors, LITAF gene inhibitors, Liver X receptor agonists and antagonists, Liver X receptor alpha inverse agonists, Liver X receptor beta inverse agonists, LPL gene stimulators, Lymphocyte function antigen-3 receptor antagonists, Lyn tyrosine kinase inhibitors, Lyn tyrosine kinase stimulators, Lysophosphatidate-1 receptor antagonists, Lysyl oxidase homolog (LOXL) 2 inhibitors, LXR inverse agonists, macrophage-drug conjugates (MDC), Macrophage inflammatory protein (MIP) 2 alpha inhibitors, MIP 2 beta inhibitors, MIP 3 alpha ligand inhibitors, Macrophage mannose receptor 1 modulators, Macrophage migration inhibitory factor inhibitors, MAdCAM inhibitors, MAdCAM modulators, MALT protein 1 inhibitors, Mannan-binding lectin serine protease-2 inhibitors, MAP kinase inhibitors, MAP kinase kinase 4 inhibitors, MAP kinase modulators, MAP3K2 gene inhibitors, MAPKAPK2 inhibitors, MAPKAPK5 inhibitors, Matrix extracell phosphoglycoprotein modulators, Matrix metalloprotease inhibitors, MCH receptor-1 antagonists, MCL1 gene inhibitors, MEK protein kinase inhibitors, MEK-1 protein kinase inhibitors, MEK-2 protein kinase inhibitors, MEKK-5 protein kinase inhibitors, melanin concentrating hormone (MCH-1) antagonists, melanocortin agonists, Melanocortin MC1 receptor agonists, Melanocortin MC3 receptor agonists, Melanocortin receptor agonists, Membrane copper amine oxidase inhibitors, Metalloprotease-1 inhibitors, Metalloprotease-2 inhibitors, Metalloprotease-9 inhibitors, Metalloprotease-9 stimulators, methylprednisolone, Methionine aminopeptidase-2 inhibitors, Methyl CpG binding protein 2 modulators, microbiome-targeting therapeutics, MicroRNA-132 (miR-132) antagonists, MicroRNA-21(miR-21) inhibitors, Midkine ligand inhibitors, Mineralocorticoid receptor antagonists and modulators, Mitochondrial uncouplers, Mitochondrial 10 kDa heat shock protein stimulators, Mitochondrial pyruvate carrier 2 inhibitors, Mitochondrial pyruvate carrier inhibitors, Mixed lineage kinase-3 inhibitors, MKL myocardin like protein inhibitors, MNK protein kinase inhibitors, Monocarboxylate transporter inhibitors, Monocyte macrophage differentiation inhibitors, Motile sperm domain protein 2 inhibitors, MST-1 protein kinase inhibitors, mTOR complex 1 inhibitors, mTOR complex 2 inhibitors, mTOR inhibitors, Myelin basic protein stimulators, Myeloperoxidase inhibitors, Myosin 2 inhibitors, N-formyl peptide receptor antagonists, NACHT LRR PYD domain protein 3 (NLRP3) inhibitors, NAD ADP ribosyltransferase stimulators, NAD-dependent deacetylase sirtuin stimulators, NAD-dependent deacetylase sirtuin-1 stimulators, NADPH oxidase inhibitors, NADPH oxidase 1 inhibitors, NADPH oxidase 4 inhibitors, NAMPT gene inhibitors, natriuretic peptide receptor C agonists, neuregulin-4 ligands, Neuropilin 2 modulators, Neutral endopeptidase inhibitors, NF kappa B inhibitor stimulators, NFAT gene inhibitors, NFE2L2 gene inhibitors, NFE2L2 gene stimulators, Nicotinic acetylcholine receptor antagonists, Nicotinic acid receptor 1 agonists, Nicotinamide phosphoribosyltransferase inhibitors, NK cell receptor modulators, NK1 receptor antagonists, NKG2 A B activating NK receptor antagonists, NKG2 D activating NK receptor antagonists, NLR family member X1 stimulators, NLRP3 inhibitors, NMDA receptor epsilon 2 subunit inhibitors, NOD2 gene modulators, Non receptor tyrosine kinase TYK2 antagonists, NOX4 gene inhibitors, NUAK SNF1-like protein kinase 1 inhibitors, Nuclear erythroid 2-related factor 2 stimulators, Nuclear factor kappa (NEK) B inhibitors and modulators, Nuclear factor kappa B p105 inhibitors, nuclear hormone receptor modulators, Nuclear pore complex protein modulators, Nuclear receptor modulators, Nuclease stimulators, Nucleoside reverse transcriptase inhibitors, Nucleosome assembly protein 1 like-4 inhibitors, Oncostatin M receptor modulators, Oncostatin M receptor subunit beta inhibitors, opioid receptor antagonists, Opioid growth factor receptor agonists, Opioid receptor delta, kappa, and mu antagonists, Opioid receptor sigma antagonist 1, Orphan nuclear receptor antagonists, Osteoclast differentiation factor antagonists, Osteoclast differentiation factor ligand inhibitors, Oxidoreductase inhibitors, OX40 ligand inhibitors, OX-40 receptor antagonists and modulators, Oxyntomodulin ligands, PGE1 agonists, P-Glycoprotein inhibitors, P-selectin glycoprotein ligand-1, 14-3-3 protein eta inhibitors, P2X3 purinoceptor antagonists, P2X7 purinoceptor agonists and modulators, P2Y6 purinoceptor modulators, P2Y13 purinoceptor stimulators, p38 MAP kinase alpha inhibitors, p38 MAP kinase inhibitors, p53 tumor suppressor protein stimulators, PACAP type I receptor agonists, Pan cathepsin inhibitors, Parathyroid hormone ligand inhibitors, PARP modulators, PDE 1 inhibitors, PDE 3 inhibitors, PDE 4 inhibitors, PDE 4b inhibitors, PDE 5 inhibitors, PDGF-B ligand inhibitors, PDGF receptor agonists, PDGF receptor alpha antagonists, PDGF receptor beta antagonists and modulators, PEGylated long-acting glucagon-like peptide-1/glucagon (GLP-1R/GCGR) receptor dual agonists, Pellino homolog 1 inhibitors, Peptidyl-prolyl cis-trans isomerase A inhibitors, Peptidyl-prolyl cis-trans isomerase D inhibitors, PERK gene inhibitors, PGI2 agonists, PGD2 antagonists, Phenylalanine hydroxylase stimulators, Phosphatidylinositol 3 kinase subunit 3 inhibitors, Phosphatonin receptor agonists, Phosphoinositide 3-kinase inhibitors, Phosphoinositide-3 kinase alpha, delta, and gamma inhibitors, Phospholipase A2 inhibitors, Phospholipase C inhibitors, Phosphoric diester hydrolase inhibitors, Phosphorylase inhibitors, Plasma retinol binding protein inhibitors, Plasminogen activator inhibitor 1 inhibitors, Plasmin stimulators, Platelet activating factor receptor antagonists, Plexin domain containing protein stimulators, PNPLA3 gene inhibitors and modulators, Potassium channel inhibitors PPAR agonists, PPAR alpha/delta agonists, PPAR delta agonists, PPAR gamma agonists and modulators, PRKAA2 gene stimulators, Programmed cell death ligand (PDL) 1 modulators, Programmed cell death protein 1 modulators, Programmed cell death protein 1 stimulators, Proprotein convertase PC9 inhibitors, Prostacyclin (PGI2) agonists, Prostaglandin D synthase stimulators, Prostanoid receptor antagonists, Protease-activated receptor-2 antagonists, Proteasome beta-8 subunit modulators, Proteasome inhibitors, Protein arginine deiminase inhibitors, Protein arginine deiminase IV inhibitors, Protein C activators, Protein cereblon modulators, protein fimH inhibitors, Protein kinase C theta inhibitors, Protein kinase inhibitors and modulators, Protein kinase C theta inhibitors, Protein MB21D1 inhibitors and modulators, Protein NOV homolog modulators, P-selectin glycoprotein ligand-1 inhibitors, Protein tyrosine kinase inhibitors, Protein tyrosine phosphatase beta inhibitors, Protein tyrosine phosphatase-iB inhibitors, Protein tyrosine phosphatase-2C inhibitors, Protein tyrosine phosphatase lE inhibitors, P-selectin glycoprotein ligand-1 stimulators, PTGS2 gene inhibitors, PurH purine biosynthesis protein inhibitors, QSK serine threonine protein kinase inhibitors, Ras gene inhibitors, Reactive oxygen species modulator inhibitors, Relaxin receptor modulators, Relaxin receptor 2 modulators, Renin inhibitors, Resistin ligand inhibitors, Resistin/CAP1 (adenylyl cyclase associated protein 1) interaction inhibitors, Retinoic acid receptor agonists, Retinoic acid receptor gamma antagonists and inverse agonists, Retinoid receptor agonists, Retinoid X receptor agonists and modulators, Retinoid Z receptor gamma agonists and antagonists, Ret tyrosine kinase receptor inhibitors, Rev protein modulators, Rho associated protein kinase inhibitors, Rho associated protein kinase 1 inhibitors, Rho associated protein kinase 2 inhibitors, Rhomboid family member 2 inhibitors, Ribonuclease P inhibitors, RIP-1 kinase inhibitors, RIP-2 kinase inhibitors, RNA polymerase inhibitors, Seprase inhibitors, Serine threonine protein kinase TBK1 inhibitors, Serine threonine protein kinase TBK1 modulators, Serine threonine SNF1 like kinase 2 inhibitors, SERPINHI gene inhibitors, Serum amyloid A protein modulators, Serum amyloid P stimulators, Signal transducer CD24 modulators, Signal transduction inhibitors, SLC22A12 inhibitors, SMAD inhibitors, SMAD-3 inhibitors, Smoothened receptor antagonists, S-nitrosoglutathione reductase (GSNOR) enzyme inhibitors, Sodium channel inhibitors, Sodium glucose transporter-1 inhibitors, Sodium glucose transporter-2 inhibitors, Solute carrier family inhibitors, Somatostatin receptor agonists, Sphingolipid delta 4 desaturase DES1 inhibitors, Sphingosine kinase 1 inhibitors, Sphingosine kinase 2 inhibitors, Sphingosine 1 phosphate phosphatase modulators, sphingosine 1 phosphate phosphatase 1 stimulators, sphingosine-1-phosphate receptor-1 agonists, sphingosine-1-phosphate receptor-5 agonists, sphingosine-1-phosphate receptor-1 antagonists, sphingosine-1-phosphate receptor-1 modulators, Sphingosine-1-phosphate receptor-3 modulators, Sphingosine-1-phosphate receptor-4 modulators, Sphingosine-1-phosphate receptor-5 modulators, Src tyrosine kinase inhibitors, SREBP transcription factor inhibitors, SREBP transcription factor 1 inhibitors, SREBP transcription factor 2 inhibitors, STAT inhibitors, STAT3 gene inhibitors, STAT-1 inhibitors and modulators, STAT-3 inhibitors and modulators, STAT-5 inhibitors, STAT-6 inhibitors, Stearoyl CoA desaturase-1 inhibitors, stem cell antigen-1 inhibitors, Stimulator of interferon genes protein inhibitors, STK25 inhibitors, Stress induced secreted protein 1 stimulators, superoxide dismutase modulators, Superoxide dismutase stimulators, Suppressor of cytokine signalling-1 stimulators, Suppressor of cytokine signalling-3 stimulators, SYK inhibitors, Syndecan-1 inhibitors, TACE inhibitors, TAK1 binding protein modulators, Talin modulators, Taste receptor type 2 agonists, T-box transcription factor TBX21 modulators, T-cell differentiation antigen CD6 inhibitors, T cell receptor modulators, T cell receptor antagonists, T-cell surface glycoprotein CDla inhibitors, T-cell surface glycoprotein CD8 inhibitors, T cell surface glycoprotein CD28 inhibitors, T-cell surface glycoprotein CD8 modulators, T cell surface glycoprotein CD28 stimulators, T-cell transcription factor NFAT modulators, Tec tyrosine kinase inhibitors, Telomerase stimulators, Tenascin modulators, TERT gene modulators, TGF-beta activated kinase-1 inhibitors, TGF-beta activation modulators, TGF beta agonists, TGF beta ligand inhibitors, TGF beta 1 ligand inhibitors, TGF beta 3 ligand inhibitors, TGF beta 1 gene inhibitors, TGF beta 1 ligand modulators, TGF beta receptor antagonists, TGF beta receptor antagonists, TGF-beta type II receptor antagonists, TGFB1 gene inhibitors, Thioredoxin reductase inhibitors, Thrombomodulin stimulators, Thromboxane A2 antagonists, Thromboxane A2 receptor antagonists, Thromboxane synthesis inhibitors, Thymic stromal lymphopoietin ligand inhibitors, Thymic stromal lymphopoietin ligand modulators, Thymic stromal lymphopoietin receptor modulators, Thymulin agonists, Thyroid hormone receptor agonists, Thyroid hormone receptor beta agonists, tissue transglutaminase inhibitors, Toll-like receptor (TLR)-2 antagonists, TLR-3 antagonists, TLR-4 antagonists, TLR-7 antagonists and modulators, TLR-8 antagonists, TLR-9 antagonists and agonists, TLR modulators, TNF alpha ligand agonists and antagonists, TNF ligand agonists and antagonists, TNF binding agents, TNF gene inhibitors, TNFSF1 I gene inhibitors, Topoisomerase II inhibitors, TPL-2 inhibitors, Transaminase stimulators, Transcription factor modulators, Transcription factor β65 inhibitors, Transcription factor RelB inhibitors, Transferrin modulators, Transforming growth factor β (TGF-0) Transforming growth factor R activated Kinase 1 (TAK1), Transglutaminase inhibitors, Transthyretin modulators, TrkA receptor antagonists, Trk tyrosine kinase receptor inhibitors, TRP cation channel Al inhibitors, TRP cation channel C5 inhibitors, TRP cation channel C6 inhibitors, Tryptophan 5-hydroxylase-1 inhibitors, Tryptophanase inhibitors, Tubulin binding agents, Tumor necrosis factor ligand inhibitors, Tumor necrosis factor ligand 13 inhibitors, Tumor necrosis factor 15 ligand inhibitors, tumor necrosis factor 14 ligand modulators, Tumor necrosis factor 13C receptor antagonists, Tumor necrosis factor 14 ligand inhibitors, Tyk2 tyrosine kinase inhibitors, Type I IL-1 receptor antagonists, Type I TNF receptor antagonists, Type II TNF receptor antagonists, Type II TNF receptor modulators, Tyrosine kinase receptor inhibitors, Tyrosine kinase receptor modulators, Ubiquitin ligase modulators and stimulators, Ubiquitin thioesterase-30 inhibitors, Uncoupling protein modulators, Unspecified cell adhesion molecule inhibitors, Unspecified GPCR agonists, Unspecified GPCR modulators, Unspecified growth factor receptor antagonists, Urate anion exchanger 1 inhibitors, vanilloid VR1 agonists, Vanilloid VR1 antagonists, Vasopressin Vla receptor antagonists, VDR agonists, VEGF receptor antagonists, VEGF receptor modulators, VEGF-1 receptor antagonists, VEGF-2 receptor antagonists, VEGF-3 receptor antagonists, VEGF-2 receptor modulators, VEGF-B ligand inhibitors, Vimentin inhibitors, VIP 1 receptor agonists, VIP 2 receptor agonists, Vitamin D3 receptor agonists, Vitamin D3 receptor modulators, Vitamin K dependent protein C stimulators, WNT modulators, Wnt ligand inhibitors, Wnt 5A ligand inhibitors, Xanthine oxidase inhibitors, X-linked inhibitor of apoptosis protein inhibitors, XPO1 gene modulators, YAP/TAZ modulators, YSK-4 protein kinase inhibitors, Zap70 tyrosine kinase inhibitors, Zinc finger binding protein Aiolos inhibitors, and zonulin inhibitors.

196.-198. (canceled)

199. The method of claim 143, wherein the subject is a human subject.

200. A method of downregulating an immune response in a subject, comprising administering the subject the antibody or antigen-binding fragment thereof of claim 2 or an immunoconjugate comprising the antibody or antigen-binding fragment thereof, or administering to the subject a pharmaceutical composition comprising the antibody or antigen-binding fragment thereof, or the immunoconjugate.

201. A method of suppressing an immune cell that expresses CD200R, comprising contacting said immune cell with the antibody or antigen-binding fragment thereof of claim 2 or an immunoconjugate comprising the antibody or antigen-binding fragment thereof; optionally wherein said immune cell comprises a T cell, a B cell, or a macrophage or an antigen-specific T cell: and/or
wherein the subject is a human subject.

202.-205. (canceled)


SEQUENCE LISTING (98 sequences)

SEQ ID NO: 1 - protein, 114 aa, synthetic construct
QVQLQQPGSELVRPGASVKLSCKASGHTFTSYWMHWVKQRPGQGLEWIGNIYPGSGSINYDEKFKSKAKLTVDTSSRTAYMQLSSLTSEDSAVYYCLTMTGTSWGQGTLVTVSA

SEQ ID NO: 2 - protein, 107 aa, synthetic construct
DVQMIQSPSSLSASLGDIVTMTCQASQGTPINLHWFQQKPGKAPKLLISGASNLEDGVPSRFSGSRYGTAFTLTISSLEDEDMATYFCLQFTYIPWTFGGGTKLEIK

SEQ ID NO: 3 - protein, 5 aa, synthetic construct
SYWMH

SEQ ID NO: 4 - protein, 17 aa, synthetic construct
NIYPGSGSINYDEKFKS

SEQ ID NO: 5 - protein, 5 aa, synthetic construct
MTGTS

SEQ ID NO: 6 - protein, 11 aa, synthetic construct
QASQGTPINLH

SEQ ID NO: 7 - protein, 7 aa, synthetic construct
GASNLED

SEQ ID NO: 8 - protein, 9 aa, synthetic construct
LQFTYIPWT

SEQ ID NO: 9 - protein, 119 aa, synthetic construct
EVQLQQSGPELVKPGASVKMSCKASGYTFTSYVMHWVKQKPGQGLEWIGYIHPYNDDIKHNEKFKDKATLTSAKSSSTVYMELSSLTSEDSAVYYCAREEYYGSRFAYWGQGTLVTVSA

SEQ ID NO: 10 - protein, 111 aa, synthetic construct
DIVLTQSPASLAVSLGQRATISCRASKSVRTSGYSYLHWYQQKPGQPPKLLIYLASNLESGVPARFSGSGSGTDFTLNIHPVEEEDAATYYCQYSGELPFTFGGGTKLEIK

SEQ ID NO: 11 - protein, 5 aa, synthetic construct
SYVMH

SEQ ID NO: 12 - protein, 17 aa, synthetic construct
YIHPYNDDIKHNEKFKD

SEQ ID NO: 13 - protein, 10 aa, synthetic construct
EEYYGSRFAY

SEQ ID NO: 14 - protein, 15 aa, synthetic construct
RASKSVRTSGYSYLH

SEQ ID NO: 15 - protein, 7 aa, synthetic construct
LASNLES

SEQ ID NO: 16 - protein, 9 aa, synthetic construct
QYSGELPFT

SEQ ID NO: 17 - protein, 117 aa, synthetic construct
QVQLKESGPGLVAPSQSLSITCTVSGFSLTSYGVHWVRQLPGKGLEWLGVIWAGGGTNYNSALMSRLSISKDNSKSQVFLKMNRLQTDDTAIYYCAREGLPRAMDYWGQGTSVTVSS

SEQ ID NO: 18 - protein, 107 aa, synthetic construct
DIVLTQSPASLAVSLGQRATISCRASESVDNYGISFMNWFQQKPGQPPKLLSNQGSGVPARFSGSGSGTDFSLNIHPMEEDDTAMYFCQQGKEFPWTFGGGTKLEIN

SEQ ID NO: 19 - protein, 5 aa, synthetic construct
SYGVH

SEQ ID NO: 20 - protein, 16 aa, synthetic construct
VIWAGGGTNYNSALMS

SEQ ID NO: 21 - protein, 9 aa, synthetic construct
EGLPRAMDY

SEQ ID NO: 22 - protein, 15 aa, synthetic construct
RASESVDNYGISFMN

SEQ ID NO: 23 - protein, 7 aa, synthetic construct
QGSGVPA

SEQ ID NO: 24 - protein, 9 aa, synthetic construct
QQGKEFPWT

SEQ ID NO: 25 - protein, 114 aa, synthetic construct
QVQLQQPGSELVRPGASVKLSCKASGYTFTSYWMHWVKQRPGQGLEWIGNIFPGSDTTNYDEKFKSKAIMTVDISSSTVYMHLSSLTSEDSAVYYCITYTGAYWGQGTLVTVSA

SEQ ID NO: 26 - protein, 107 aa, synthetic construct
DVQMIQSPSSLSASMGDIVTMTCQASQGTNINLHWFQQKPGKAPKLLISGGSNLEDGVPSRFSGSRYGTDFTLTISSLEDEDMATYFCLQFTYLPWTFGGGTKLDIK

SEQ ID NO: 27 - protein, 5 aa, synthetic construct
SYWMH

SEQ ID NO: 28 - protein, 17 aa, synthetic construct
NIFPGSDTTNYDEKFKS

SEQ ID NO: 29 - protein, 5 aa, synthetic construct
YTGAY

SEQ ID NO: 30 - protein, 11 aa, synthetic construct
QASQGTNINLH

SEQ ID NO: 31 - protein, 7 aa, synthetic construct
GGSNLED

SEQ ID NO: 32 - protein, 9 aa, synthetic construct
LQFTYLPWT

SEQ ID NO: 33 - protein, 114 aa, synthetic construct
QVQLQQPGSELVRPGASVKLSCKASGYTFTSYWMHWVKQRPGQGLEWIGNIYPGSGTTNYDEKFKSKATLTVDTSSSTAYMQISSLTSEDSAVYYCTTGTSTYWGQGTLVTVSA

SEQ ID NO: 34 - protein, 107 aa, synthetic construct
DVQMIQSPSSLSASLGDIVTMTCQASQGTSINLNWFQQKPGKAPKLLIYGAVNLEDGVPSRFSGSRYGTDFTLTVSSLEDEDMATYFCLQHTYLPWTFGGGTKLEIK

SEQ ID NO: 35 - protein, 5 aa, synthetic construct
SYWMH

SEQ ID NO: 36 - protein, 17 aa, synthetic construct
NIYPGSGTTNYDEKFKS

SEQ ID NO: 37 - protein, 5 aa, synthetic construct
GTSTY

SEQ ID NO: 38 - protein, 11 aa, synthetic construct
QASQGTSINLN

SEQ ID NO: 39 - protein, 7 aa, synthetic construct
GAVNLED

SEQ ID NO: 40 - protein, 9 aa, synthetic construct
LQHTYLPWT

SEQ ID NO: 41 - protein, 17 aa, synthetic construct
NIYPGSGSINYDEKFKS

SEQ ID NO: 42 - protein, 114 aa, synthetic construct
QVQLVQSGSELKKPGASVKLSCKASGHTFTSYWMHWVKQAPGQGLEWIGNIYPGSGSINYDEKFQGRATLTVDTSTRTAYMELSSLRSEDSAVYYCLTMTGTSWGRGTLVTVSS

SEQ ID NO: 43 - protein, 114 aa, synthetic construct
QVQLVQSGAEVKKPGASVKLSCKASGHTFTSYWMHWVKQAPGQGLEWIGNIYPGSGSINYDEKFQGRATLTVDTSTRTAYMELSSLRSEDTAVYYCLTMTGTSWGRGTLVTVSS

SEQ ID NO: 44 - protein, 114 aa, synthetic construct
QVQLVQSGAEVKKPGASVKVSCKASGHTFTSYWMHWVRQAPGQGLEWIGNIYPGSGSINYDEKFQGRVTMTVDTSTSTAYMELSSLRSEDTAVYYCLTMTGTSWGRGTLVTVSS

SEQ ID NO: 45 - protein, 114 aa, synthetic construct
QVQLVQSGAEVKKPGASVKVSCKASGHTFTSYWMHWVRQAPGQGLEWMGNIYPGSGSINYDEKFQGRVTMTADTSTSTVYMELSSLRSEDTAVYYCLTMTGTSWGRGTLVTVSS

SEQ ID NO: 46 - protein, 114 aa, synthetic construct
QVQLVQSGAEVKKPGASVKVSCKASGHTFTSYWMHWVRQAPGQGLEWMGNIYPGSGSINYDEKFQGRGTITTDTSTTTRYMELSSLRSEDTAVYYCLTMTGTSWGRGTLVTVSS

SEQ ID NO: 47 - protein, 114 aa, synthetic construct
QVQLVQSGAEVKKPGASVKVSCKASGHTFTSYWMHWVRQAPGQGLEWMGNIYPGSGSINYDEKFQGRGTITTDTSTTTRYMELSSLRSEDTAVYYCTTMTGTSWGRGTLVTVSS

SEQ ID NO: 48 - protein, 107 aa, synthetic construct
DVQMTQSPSSLSVSVGDRVTMTCQASQGTPINLHWFQQKPGKAPKLLISGASNLEDGVPSRFSGSRYGTAFTLTISSLQDEDIATYFCLQFTYIPWTFGQGTKLEIK

SEQ ID NO: 49 - protein, 107 aa, synthetic construct
DVQMTQSPSSLSVSVGDRVTMTCQASQGTPINLHWFQQKPGKAPKLLISGASNLEDGVPSRFSGSRYGTAFTLTISSLQPEDIATYFCLQFTYIPWTFGQGTKLEIK

SEQ ID NO: 50 - protein, 107 aa, synthetic construct
DVQMTQSPSSLSVSVGDRVTITCQASQGTPINLHWFQQKPGKAPKLLISGASNLEDGVPSRFSGSRYGTDFTFTISSLQPEDIATYFCLQFTYIPWTFGQGTKLEIK

SEQ ID NO: 51 - protein, 107 aa, synthetic construct
DIQMTQSPSSLSVSVGDRVTITCQASQGTPINLHWFQQKPGKAPKLLISGASNLEDGVPSRFSGSGSGTDFTFTISSLQPEDIATYYCLQFTYIPWTFGQGTKLEIK

SEQ ID NO: 52 - protein, 107 aa, synthetic construct
DIQMTQSPSSLSVSVGDRVTITCQASQGTPINLHWFQQKPGKAPKLLISGASNLEDGVPSRFSGSGSGTEFTFTISSLQPEDAATYYCLQFTYIPWTFGQGTKLEIK

SEQ ID NO: 53 - protein, 120 aa, synthetic construct
QVQLVQSGAEVKKPGASVKVSCKASGFSFSSGYYMAWVRQAPGQGLEWMGLIGVGSGSLWYAQKFQGRVTMTRDTSTSTVYMELSSLRSEDTAVYYCARHFALSDPFNLWGQGTLVTVSS

SEQ ID NO: 54 - protein, 108 aa, synthetic construct
EIVLTQSPDFQSVTPKEKVTITCQASESIDSYLLWYQQKPDQSPKLLIKQASTLASGVPSRFSGSGSGTDFTLTINSLEAEDAATYYCQNYYDISSNDFGGGTKVEIK

SEQ ID NO: 55 - protein, 6 aa, synthetic construct
SGYYMA

SEQ ID NO: 56 - protein, 17 aa, synthetic construct
LIGVGSGSLWYAQKFQG

SEQ ID NO: 57 - protein, 10 aa, synthetic construct
HFALSDPFNL

SEQ ID NO: 58 - protein, 11 aa, synthetic construct
QASESIDSYLL

SEQ ID NO: 59 - protein, 7 aa, synthetic construct
QASTLAS

SEQ ID NO: 60 - protein, 10 aa, synthetic construct
QNYYDISSND

SEQ ID NO: 61 - protein, 330 aa, unidentified
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 107 aa, Homo sapiens [sequence omitted]

SEQ ID NO: 63 - protein, 324 aa, unidentified
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 64 - protein, 107 aa, unidentified
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 65 - protein, 107 aa, synthetic construct
DIQMTQSPSSLSVSVGDRVTITCQASQGTPINLHWFQQKPGKAPKLLISGASNLESGVPSRFSGSGSGTEFTFTISSLQPEDAATYYCLQFTYIPWTFGQGTKLEIK

SEQ ID NO: 66 - protein, 107 aa, synthetic construct
DIQMTQSPSSLSVSVGDRVTITCQASQGTPINLHWFQQKPGKAPKLLISGASNLETGVPSRFSGSGSGTEFTFTISSLQPEDAATYYCLQFTYIPWTFGQGTKLEIK

SEQ ID NO: 67 - protein, 7 aa, unidentified
GASNLES

SEQ ID NO: 68 - protein, 7 aa, unidentified
GASNLET

SEQ ID NO: 69 - protein, 5 aa, synthetic construct
  SITE 1: note = X is M or G
XTGTS

SEQ ID NO: 70 - protein, 5 aa, synthetic construct
GTGTS

SEQ ID NO: 71 - protein, 114 aa, synthetic construct
EVQLVQSGAEVKKPGASVKVSCKASGHTFTSYWMHWVRQAPGQGLEWMGNIYPGSGSINYDEKFQGRVTMTADTSTSTVYMELSSLRSEDTAVYYCLTMTGTSWGRGTLVTVSS

SEQ ID NO: 72 - protein, 114 aa, synthetic construct
EVQLVQSGAEVKKPGASVKVSCKASGHTFTSYWMHWVRQAPGQGLEWMGNIYPGSGSINYDEKFQGRVTMTADTSTSTVYMELSSLRSEDTAVYYCLTGTGTSWGRGTLVTVSS

SEQ ID NO: 73 - protein, 446 aa, synthetic construct
QVQLVQSGAEVKKPGASVKVSCKASGFSFSSGYYMAWVRQAPGQGLEWMGLIGVGSGSLWYAQKFQGRVTMTRDTSTSTVYMELSSLRSEDTAVYYCARHFALSDPFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 74 - protein, 215 aa, synthetic construct
EIVLTQSPDFQSVTPKEKVTITCQASESIDSYLLWYQQKPDQSPKLLIKQASTLASGVPSRFSGSGSGTDFTLTINSLEAEDAATYYCQNYYDISSNDFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 77 - protein, 330 aa, synthetic construct
  SITE 121: note = X is D or P
  SITE 329: note = X is G or absent
  SITE 330: note = X is K or absent
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGXSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 78 - protein, 327 aa, synthetic construct
  SITE 327: note = X is K or absent
  SITE 326: note = X is G or absent
  SITE 108: note = X is S or P
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPXCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLXX

SEQ ID NO: 79 - protein, 444 aa, synthetic construct
QVQLVQSGAEVKKPGASVKVSCKASGHTFTSYWMHWVRQAPGQGLEWMGNIYPGSGSINYDEKFQGRVTMTADTSTSTVYMELSSLRSEDTAVYYCLTMTGTSWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 444 aa, synthetic construct
EVQLVQSGAEVKKPGASVKVSCKASGHTFTSYWMHWVRQAPGQGLEWMGNIYPGSGSINYDEKFQGRVTMTADTSTSTVYMELSSLRSEDTAVYYCLTMTGTSWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 444 aa, synthetic construct
EVQLVQSGAEVKKPGASVKVSCKASGHTFTSYWMHWVRQAPGQGLEWMGNIYPGSGSINYDEKFQGRVTMTADTSTSTVYMELSSLRSEDTAVYYCLTGTGTSWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 444 aa, Homo sapiens
EVQLVQSGAEVKKPGASVKVSCKASGHTFTSYWMHWVRQAPGQGLEWMGNIYPGSGSINYDEKFQGRVTMTADTSTSTVYMELSSLRSEDTAVYYCLTGTGTSWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 441 aa, Homo sapiens
EVQLVQSGAEVKKPGASVKVSCKASGHTFTSYWMHWVRQAPGQGLEWMGNIYPGSGSINYDEKFQGRVTMTADTSTSTVYMELSSLRSEDTAVYYCLTGTGTSWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - protein, 444 aa, Homo sapiens
EVQLVQSGAEVKKPGASVKVSCKASGHTFTSYWMHWVRQAPGQGLEWMGNIYPGSGSINYDEKFQGRVTMTADTSTSTVYMELSSLRSEDTAVYYCLTMTGTSWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 441 aa, Homo sapiens
EVQLVQSGAEVKKPGASVKVSCKASGHTFTSYWMHWVRQAPGQGLEWMGNIYPGSGSINYDEKFQGRVTMTADTSTSTVYMELSSLRSEDTAVYYCLTMTGTSWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - protein, 214 aa, synthetic construct
DIQMTQSPSSLSVSVGDRVTITCQASQGTPINLHWFQQKPGKAPKLLISGASNLESGVPSRFSGSGSGTEFTFTISSLQPEDAATYYCLQFTYIPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - skipped

SEQ ID NO: 88 - protein, 7 aa, synthetic construct
  SITE 1: note = X is G or L
  SITE 3: note = X is D, S or T
XAXNLEX

SEQ ID NO: 89 - protein, 7 aa, synthetic construct
  SITE 1: note = X is G or L
  SITE 7: note = X is D, S or T
XASNLEX

SEQ ID NO: 90 - protein, 7 aa, synthetic construct
  SITE 2: note = X is A or G
  SITE 7: note = X is D, S or T
GXSNLEX

SEQ ID NO: 91 - protein, 9 aa, synthetic construct
  SITE 8: note = X is W or F
LQFTYIPXT

SEQ ID NO: 92 - protein, 5 aa, synthetic construct
  SITE 3: note = X is W or F
SYXMH

SEQ ID NO: 93 - protein, 114 aa, synthetic construct
  SITE 33: note = X is W or F
  SITE 99: note = X is M or G
  SITE 1: note = X is Q or E
XVQLVQSGAEVKKPGASVKVSCKASGHTFTSYXMHWVRQAPGQGLEWMGNIYPGSGSINYDEKFQGRVTMTADTSTSTVYMELSSLRSEDTAVYYCLTXTGTSWGRGTLVTVSS

SEQ ID NO: 94 - protein, 107 aa, synthetic construct
  SITE 52: note = X is S or V
  SITE 96: note = X is W or F
  SITE 56: note = X is D, S or T
  SITE 51: note = X is A or G
  SITE 50: note = X is G or L
DIQMTQSPSSLSVSVGDRVTITCQASQGTPINLHWFQQKPGKAPKLLISXXXNLEXGVPSRFSGSGSGTEFTFTISSLQPEDAATYYCLQFTYIPXTFGQGTKLEIK

SEQ ID NO: 95 - protein, 444 aa, synthetic construct
  SITE 443: note = X is G or absent
  SITE 235: note = X is D or P
  SITE 99: note = X is M or G
  SITE 33: note = X is W or F
  SITE 1: note = X is D or E
  SITE 444: note = X is K or absent
XVQLVQSGAEVKKPGASVKVSCKASGHTFTSYXMHWVRQAPGQGLEWMGNIYPGSGSINYDEKFQGRVTMTADTSTSTVYMELSSLRSEDTAVYYCLTXTGTSWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGXSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 96 - protein, 441 aa, synthetic construct
  SITE 440: note = X is G or absent
  SITE 441: note = X is K or absent
  SITE 222: note = X is S or P
  SITE 99: note = X is M or G
  SITE 33: note = X is W or F
  SITE 1: note = X is D or E
XVQLVQSGAEVKKPGASVKVSCKASGHTFTSYXMHWVRQAPGQGLEWMGNIYPGSGSINYDEKFQGRVTMTADTSTSTVYMELSSLRSEDTAVYYCLTXTGTSWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPXCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLXX

SEQ ID NO: 97 - protein, 438 aa, unidentified
  SITE 1: note = X is D or E
  SITE 33: note = X is W or F
  SITE 99: note = X is M or G
  SITE 437: note = X is G or absent
  SITE 438: note = X is K or absent
XVQLVQSGAEVKKPGASVKVSCKASGHTFTSYXMHWVRQAPGQGLEWMGNIYPGSGSINYDEKFQGRVTMTADTSTSTVYMELSSLRSEDTAVYYCLTXTGTSWGRGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPXX

SEQ ID NO: 98 - protein, 214 aa, synthetic construct
  SITE 52: note = X is S or V
  SITE 51: note = X is A or G
  SITE 50: note = X is G or L
  SITE 96: note = X is W or F
  SITE 56: note = X is D, S or T
DIQMTQSPSSLSVSVGDRVTITCQASQGTPINLHWFQQKPGKAPKLLISXXXNLEXGVPSRFSGSGSGTEFTFTISSLQPEDAATYYCLQFTYIPXTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC